(19) United States
(12) Patent Application Publication
(10) Pub. No.: US 2026/0327204 A1
(43) Pub. Date: Oct. 22, 2026

(54) DEGRADERS OF CYCLIN-DEPENDENT KINASE 12/13, COMPOSITIONS, AND USES THEREOF

(71) Applicant: ZHEJIANG UNIVERSITY, Hangzhou (CN)

(72) Inventors: Chengliang ZHU, Hangzhou (CN); Bo YANG, Hangzhou (CN); Ji CAO, Hangzhou (CN); Qiaojun HE, Hangzhou (CN); Xuejing SHAO, Hangzhou (CN); Meidan YING, Hangzhou (CN); Li JIANG, Hangzhou (CN); Danni CHEN, Hangzhou (CN)

(21) Appl. No.: 19/479,084

(22) PCT Filed: Apr. 24, 2024
(86) PCT No.: PCT/CN2024/089497

§ 371 Date: Oct. 27, 2025

(30) Foreign Application Priority Data
Apr. 28, 2023 (CN) 202310479552.4

(51) Int. Cl. C07D 401/14; A61K 31/517; A61K 38/00; A61P 35/00; A61P 35/02; C07D 417/14; C07D 471/10; C07D 487/04; C07D 487/08; C07K 5/062

(52) U.S. Cl. CPC C07D 401/14; A61K 31/517; A61P 35/00; A61P 35/02; C07D 417/14; C07D 471/10; C07D 487/04; C07D 487/08; C07K 5/06034; A61K 38/00

(57) ABSTRACT

A small molecule conjugate that degrades CDK12/13, and a preparation method therefor and the use thereof. The structure of the small molecule conjugate is: T-L-E, wherein T represents a ligand of a parent nucleus molecule, i.e., a CDK12/13 protein, L represents a moiety linked to T and E, which is also referred to as linker, and E represents a ligand of an E3 ligase. The compound as represented by formula I can be synthesized by means of an amide condensation reaction, a substitution reaction or a click reaction, etc. of a derivative of the T moiety, and an E3 enzyme ligand and a derivative thereof. The compound shows an efficient CDK12/13 degradation activity on a variety of cell lines, a highly specific tumor cell proliferation inhibition activity, and an efficient treatment effect when used in combination with other anti-tumor drugs. In biological and therapeutic aspects, the compound can be used as a lead compound for treating various cancers and other CDK12/13 abnormal diseases.

### TECHNICAL FIELD

The present invention belongs to the biomedical field, and more specifically relates to a class of small molecule conjugates targeting cyclin-dependent kinase 12/13 (CDK12/13) and their uses.

### BACKGROUND ART

Cyclin-dependent kinases (CDKs) are key regulators of various cell cycle processes. They are divided into two major classes: one related to the cell cycle, such as CDK1, 2, 4, and 6, which directly regulate cell cycle progression; and another related to gene transcription, such as CDK7, 8, 9, 11, 12, and 13, which primarily regulate the process of gene transcription. Studies have found that CDK function is often dysregulated in tumor cells, making these CDKs promising therapeutic targets for such tumors.

Among these, CDK12 and CDK13 are transcription-related CDKs that form a complex with Cyclin K. By mediating the phosphorylation of serine in the C-terminal domain of RNA polymerase II (RNAPII CTD), they participate in multiple stages of gene transcription, including transcriptional elongation, mRNA splicing, and transcriptional termination. Recent studies have found that as a specific transcriptional regulator of DNA damage homologous recombination repair genes, CDK12/13 promotes the process of intron polyadenylation and plays an important role in maintaining the genomic stability of cells. Most of the kinase functions of CDK12/13 are mutually compensatory, and their simultaneous inhibition or silencing leads to the accumulation of DNA damage within cells and ultimately to cell death.

During rapid proliferation, tumor cells require a large amount of DNA replication, which causes increased DNA damage and genomic instability. Consequently, they are more dependent on the function of CDK12/13 than normal cells. There is a clear gene amplification of CDK12/13 in various tumor cells, and they have been explicitly identified as effective therapeutic targets for diseases such as breast cancer and lymphoma. The development of drugs targeting CDK12/13 not only helps to further study its biological regulatory mechanism but will also be a powerful strategy for cancer treatment. However, currently, CDK12/13 inhibitors are still in the preclinical research stage. Due to the highly similar kinase active domains of homologous protein CDKs, the developed small molecule inhibitors indiscriminately inhibit the activity of multiple CDKs. Additionally, since small molecule inhibitors lack tissue and cell targeting specificity, they have significant toxic side effects on normal cells.

PROTAC is a chemical small molecule-induced cellular protein degradation technology that leverages the naturally existing protein degradation system in the body to reduce protein levels rather than merely inhibiting the enzymatic activity of proteins, thereby achieving the goal of treating diseases. Its principle is to bridge the distance between a target protein and an E3 ubiquitin ligase to achieve ubiquitination of the target protein, thereby specifically inducing the degradation of the target protein (FIG. 1). After 20 years of continuous refinement and development, PROTAC has become a new field of drug development. Due to the subtle differences in the intracellular environment between different cell lines and the complexity of the target protein-PROTAC-E3 ligase ternary complex formation, the function of PROTAC is often accompanied by cell line-selective degradation or proliferation inhibitory activity. Therefore, the rational design and development of novel CDK12/13 protein degraders can not only treat CDK12/13-related diseases but also serve as molecular probes to study protein function.

### SUMMARY OF THE INVENTION

A primary object of the present invention is to provide a small molecule conjugate that can efficiently degrade CDK12/13 and selectively inhibit the proliferation of tumor cells. Unlike conventional inhibitors and degraders targeting CDK12/13, the small molecule conjugate provided herein efficiently degrades the target protein while exhibiting highly specific tumor cell line proliferation inhibitory activity, demonstrating properties and advantages at both the molecular and cellular levels that are distinctly different from the original parent core molecule (T in the table below, which also refers to this item hereinafter).

The small molecule conjugate for degrading CDK12/13 provided by the present invention has a structure as shown in Formula I, or is a pharmaceutically acceptable salt, co-crystal, tautomer, stereoisomer, stereoisomer mixture, solvate, hydrate, polymorph, isotopically enriched derivative, or prodrug thereof:

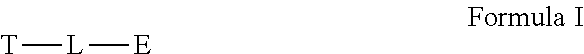

In Formula I, T represents a ligand of CDK12/13 protein, also known as a core molecule; E represents a ligand of E3 ligase; and L represents a connecting part or linker for T and E, also known as a Linker, which can be a chemical group or a chemical bond. Specifically, the said T is selected from a CDK12/13 inhibitor and a derivative thereof, and its structural group is as shown in Formula II.

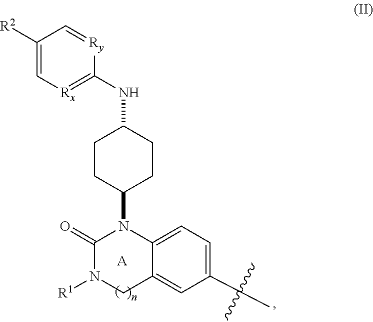

or a pharmaceutically acceptable salt, co-crystal, tautomer, stereoisomer, solvate, hydrate, polymorph, isotopically enriched derivative, or prodrug thereof, wherein:
in Formula II, R1 can be a single group (when n is not 0) or two groups (when n is 0); each R1 is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen-protecting group; alternatively or optionally, when two R1 groups are present, the two R1 groups together with the intermediate atom (N) form a substituted or unsubstituted heterocyclic group or a substituted or unsubstituted heteroaryl group;

Preferably, the specific structure of R¹ is ethyl,

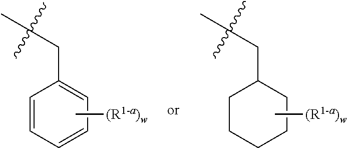

or a nitrogen-protecting group;
R^{1-a} can be multiple or one (R^{1-a} is absent when w is 0), and each R^{1-a} is independently halogen, optionally substituted acyl, or optionally substituted alkyl;
w is 0, 1, 2, 3, 4, 5, or 6.

In Formula II, R² is halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})2, or —SR^{D1}; wherein R^{D1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen-protecting group when attached to an oxygen atom, or a sulfur-protecting group when attached to a sulfur atom;
wherein each R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen-protecting group; alternatively or optionally, the two R^{D1a} groups together with the intermediate atom (C) to which they are attached form a substituted or unsubstituted heterocyclic ring or a substituted or unsubstituted heteroaryl ring;
In R¹ and R²; the said acyl is preferably C₁-C₆ acyl; the said alkyl is preferably C₁-C₆ alkyl; the said alkenyl is preferably C₁-C₆ alkenyl; the said alkynyl is preferably C₁-C₆ alkynyl; the said carbocyclyl is a 3- to 8-membered carbocyclyl; the said heterocyclyl is a 3- to 8-membered heterocyclyl; the said aryl is a 5- to 10-membered aryl; the said heteroaryl is a 5- to 10-membered heteroaryl;
The said nitrogen-protecting groups include tert-butoxycarbonyl, benzyloxycarbonyl, allyloxycarbonyl, and p-methoxybenzyl. The said oxygen-protecting groups include dihydropyran, methoxymethyl, benzyl, methoxybenzyl, trimethylsilyl, and tert-butyldimethylsilyl. The said sulfur-protecting groups include acetamidomethyl and phenylacetamidomethyl, and tert-butylthio.

The said carbocyclyl can be a 3- to 8-membered saturated carbocyclyl or a 3- to 8-membered carbocyclyl having one or two double or triple bonds.

The said optional substitution includes the case of no substitution or the case of substitution. When substitution is present, the substituent can be one or more, and the multiple substituents are each independently selected from one or more of a hydroxyl group, a cyano group, a halogen, a nitro group, a C1-C4 alkyl, a C1-C4 alkoxy, a 3- to 6-membered cycloalkyl, a 3- to 8-membered heterocyclyl, an aryl, and a heteroaryl.

Preferably, R2 is —CN.

In Formula II, Rx and Ry are each independently CH or N;
In Formula II, n is 0, 1, 2, 3, 4, or 5; when n is 0, ring A is an opened-ring chain structure, which becomes:

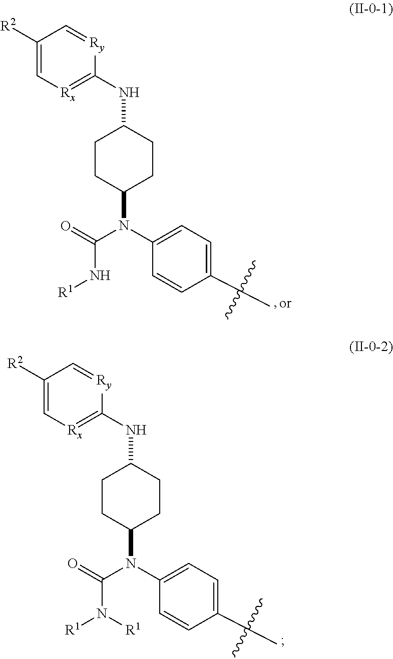

in Formula (II-0-2), the two R¹ groups are independently the same or different.

Further, the specific structure of said T can be any one of the groups shown below:

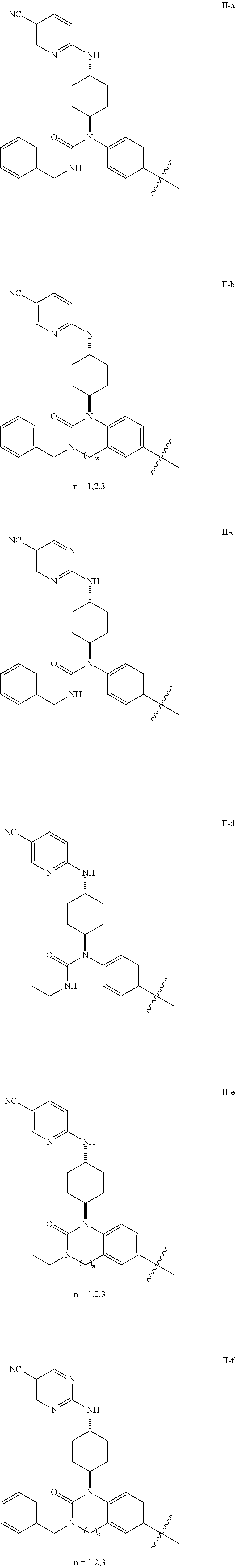

Specifically, E can be selected from the 3-amino-N-(2,6-dioxo-3-piperidinyl)phthalimide group (Pomalidomide group), the 3-(7-amino-3-oxo-1H-isoindol-2-yl)piperidine-2,6-dione group (Lenalidomide group), the thalidomide group (Thalidomide group), the (2S,4R)-1-((S)-2-(3-aminopropanamido)-3,3-dimethylbutanoyl)-4-hydroxy-N-(4-(4-methylthiazol-5-yl)benzyl)pyrrolidine-2-carboxamide group (VH032 group), the [(4R,5S)-4,5-bis(4-chlorophenyl)-2-[4-(1,1-dimethylethyl)-2-ethoxyphenyl]-4,5-dihydro-4,5-dimethyl-1H-imidazol-1-yl][4-[3-(methylsulfonyl)propyl]-1-piperazinyl]methanone group (RG-7112 group), phenylglutarimide, and the like.

For E, the said group refers to the group structure formed by removing a removable H or other leaving groups from the corresponding compound of the said group. For example, taking the 3-amino-N-(2,6-dioxo-3-piperidinyl)phthalimide group as an example, it refers to the remaining group structure in the compound 3-amino-N-(2,6-dioxo-3-piperidinyl)phthalimide after removing a removable H or other leaving groups.

As a feasible embodiment, E can be selected from one of the following groups:

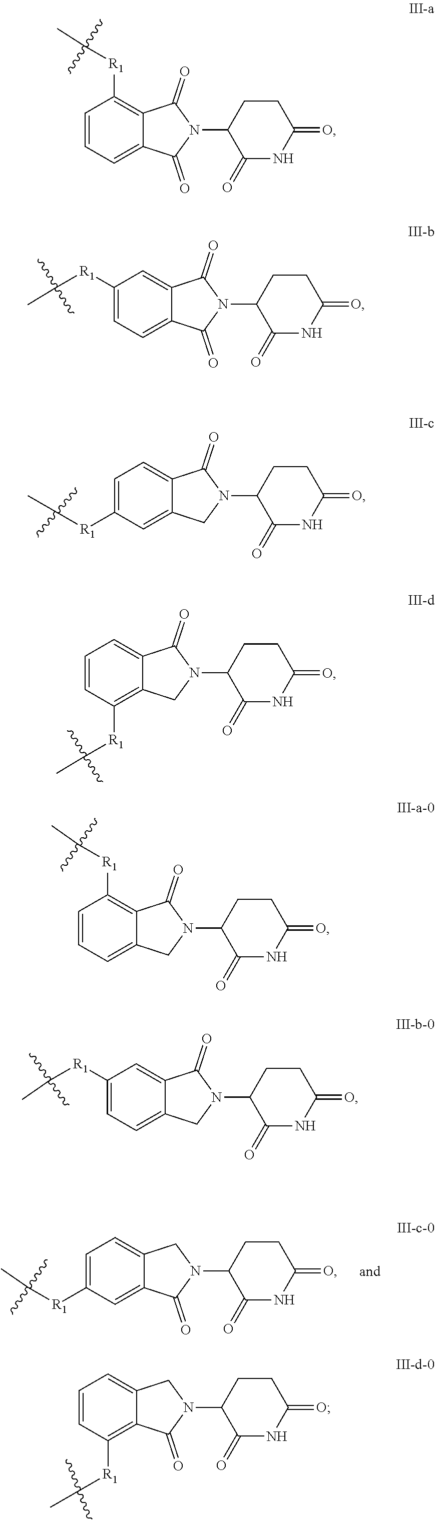

In formulae III-a to III-d and III-a-0 to III-d-0, R¹ is NH, O, or CH₂;

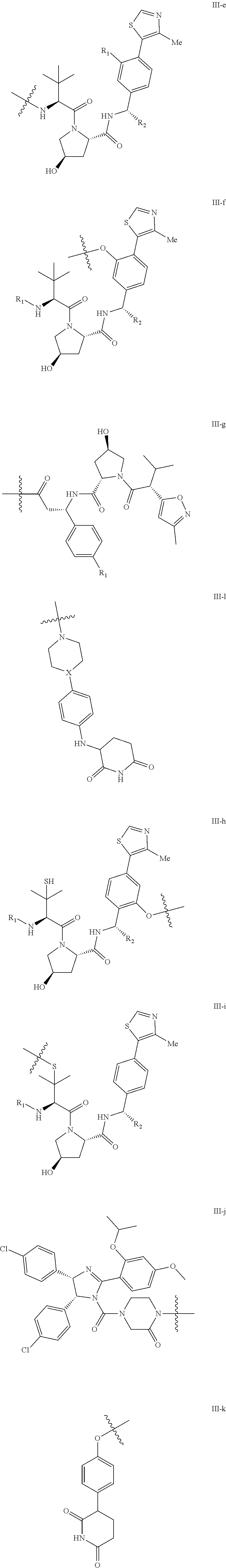

In said Formula III-e, R₁ is H or OH, and R₂ is H or CH3; In said Formula III-f, R₁ is

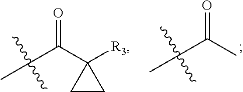

wherein R₃ is —CN, —F, —COCH₃, and R₂ is H or CH₃; In said Formula III-g, R₁ is H, CN, F, Cl, Br, or a group of the formula

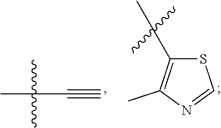

In said Formula III-h, R₁ is

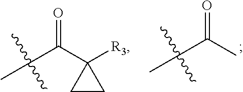

wherein R₃ is —CN, —F, —COCH₃; R₂ is H or CH₃; In said Formula III-I, R₁ is

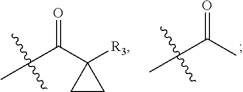

wherein R₃ is —CN, —F, —COCH₃; R₂ is H or CH₃; In said Formula III-1, X is N or CH.

Specifically, the said L is a unit of 1 to 30 atoms in length and of any type.

More specifically, the said L includes but is not limited to the following structures, or any structural group composed of the following structures:

A unit composed of an ethylene glycol structure:

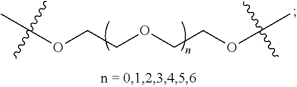

A unit composed of an aliphatic chain:

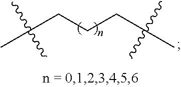

A unit composed of an unsaturated chain:

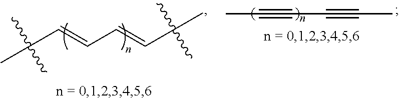

A unit composed of a non-aromatic heterocyclic ring:

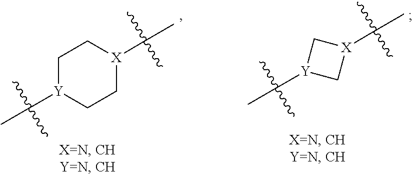

A unit composed of an aromatic compound:

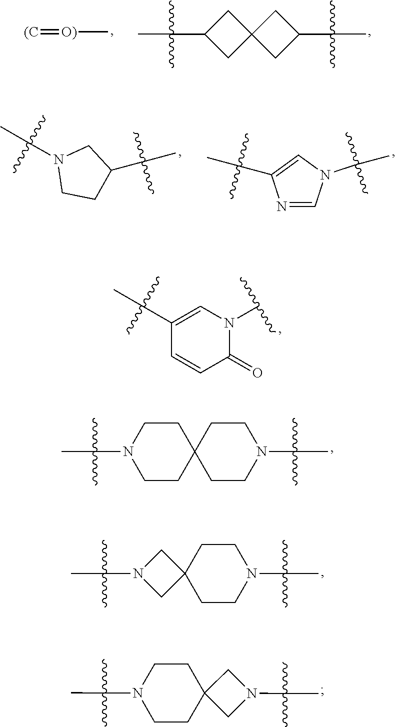

A heteroatom-containing group, wherein the heteroatoms are sulfur, nitrogen, or phosphorus, including but not limited to the following fragment units:

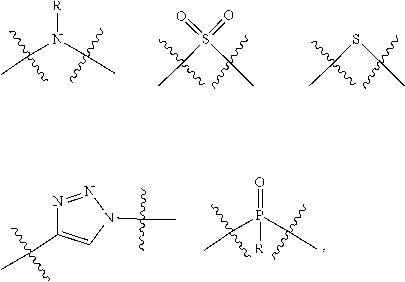

In the said heteroatom-containing unit, R is independently alkyl, alkoxy, or hydrogen. Preferably, the said alkyl is specifically methyl, ethyl, propyl, isopropyl, n-butyl, or tert-butyl; and the said alkoxy is —OEt or —OMe.

Preferably, the said L is selected from the following structures:

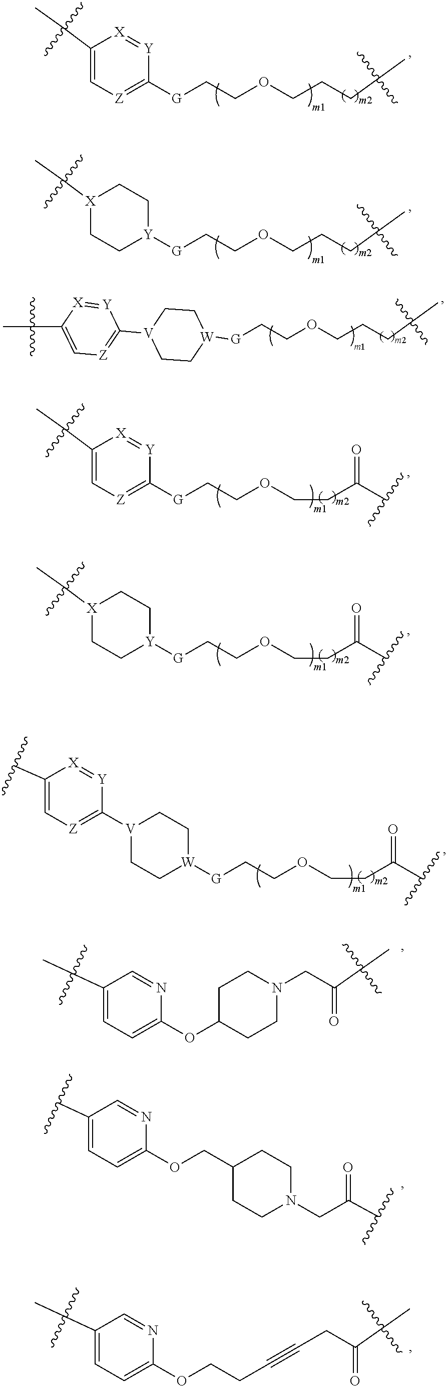

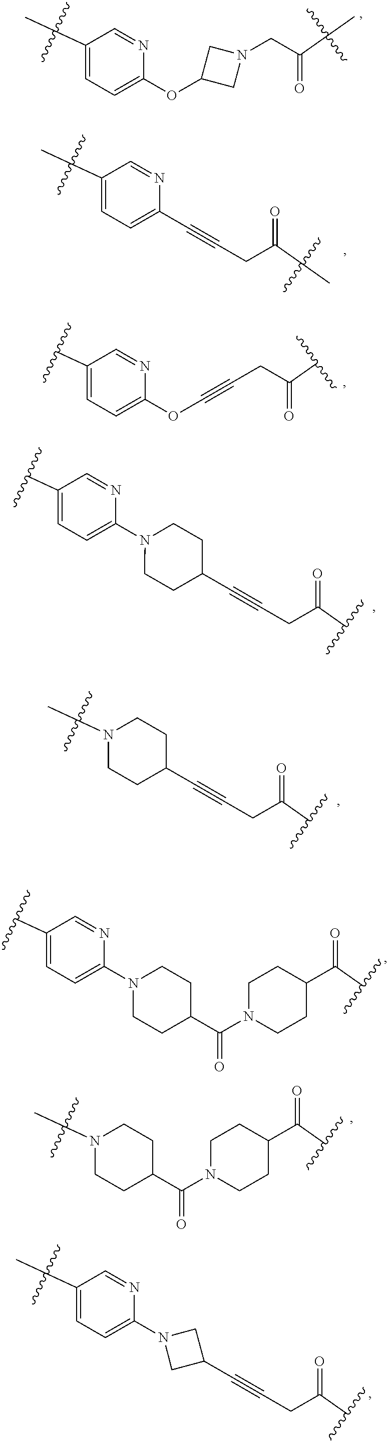

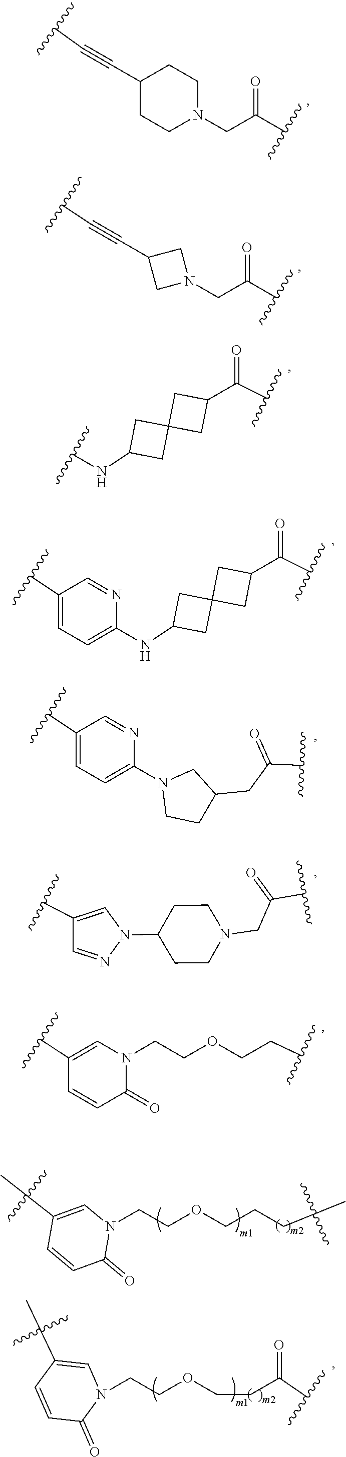

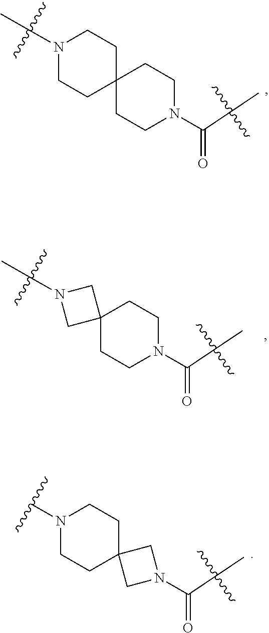 [SMILES: *C(=O)N1CCC2(CCN(*)CC2)CC1 ; *C(=O)N1CCC2(CC1)CN(*)C2 ; *C(=O)N1CC2(CCN(*)CC2)C1]

As a preferred embodiment, the said L is selected from the following structures:

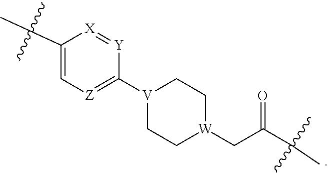

In the above structural formulae, the said X, Y, Z, V, and W are each independently selected from N and CH. As a further preferred embodiment, X, Y, and Z are not simultaneously CH.

Preferably, m₁ is 0, 1, 2, 3, 4, 5, or 6, and m₂ is 0, 1, 2, 3, 4, 5, or 6.

Specifically, the structural formula of the said small molecule conjugate is as shown in any one of formulae IV to X:

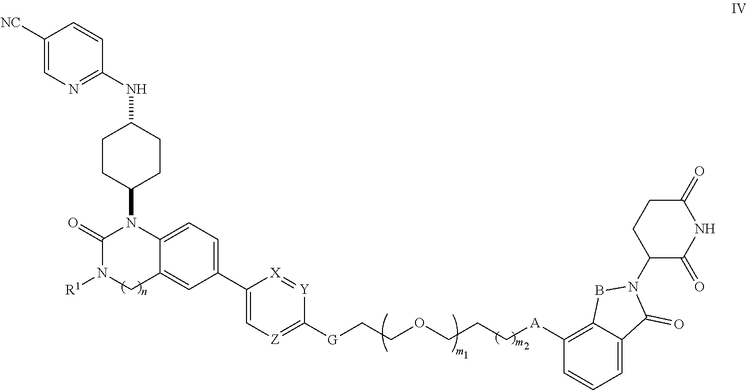

In Formula IV, m₁ is an integer from 0 to 6, m₂ is an integer from 0 to 15, A is CH₂, NH, O, or CO, G is absent, CH₂, NH, O, CONH, or NHCO, B is CO or CH₂, X, Y, and Z are each independently CH or N, and the definitions of R¹ and n are the same as in Formula II; In one embodiment, G is O.

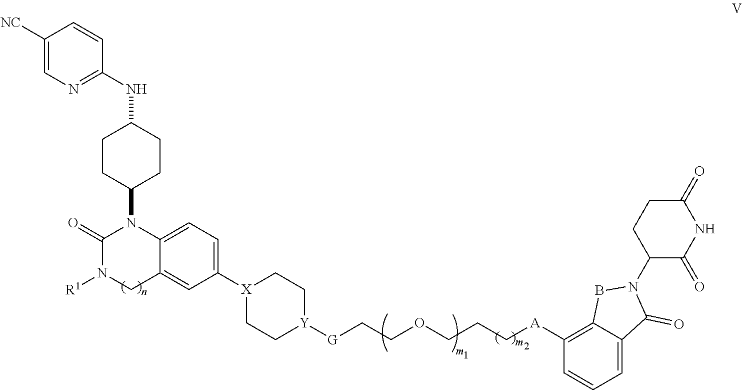

In Formula V, m₁ is an integer from 0 to 6, m₂ is an integer from 0 to 15, A is CH₂, NH, O, or CO, G is absent, CH₂, NH, O, CONH, or NHCO, B is CO or CH₂, X and Y are each independently CH or N, and the definitions of R¹ and n are the same as in Formula II; In one embodiment, when Y is N, G is absent; When Y is CH, G is a heteroatom (such as NH or O).

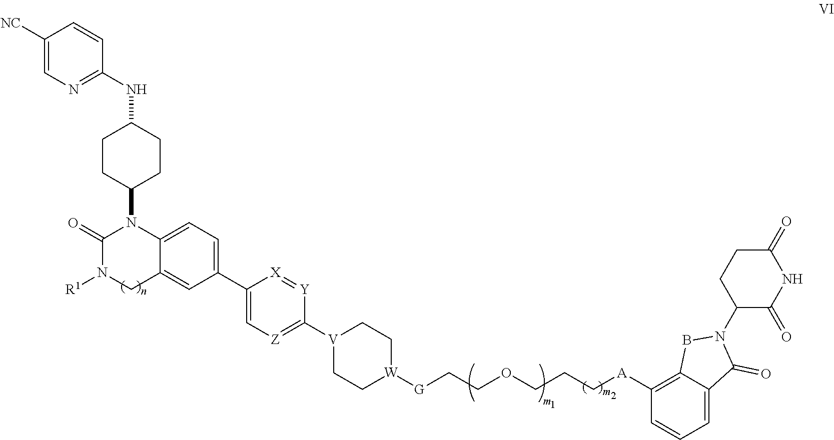

In Formula VI, m₁ is an integer from 0 to 6, m₂ is an integer from 0 to 15, A is CH₂, NH, O, or CO, G is absent, CH₂, NH, O, CONH, or NHCO, B is CO or CH₂, X, Y, and Z are each independently CH or N, V and W are each independently CH or N, and the definitions of R¹ and n are the same as in Formula II; In one embodiment, when W is N, G is absent; When W is CH, G is a heteroatom (such as NH or O).

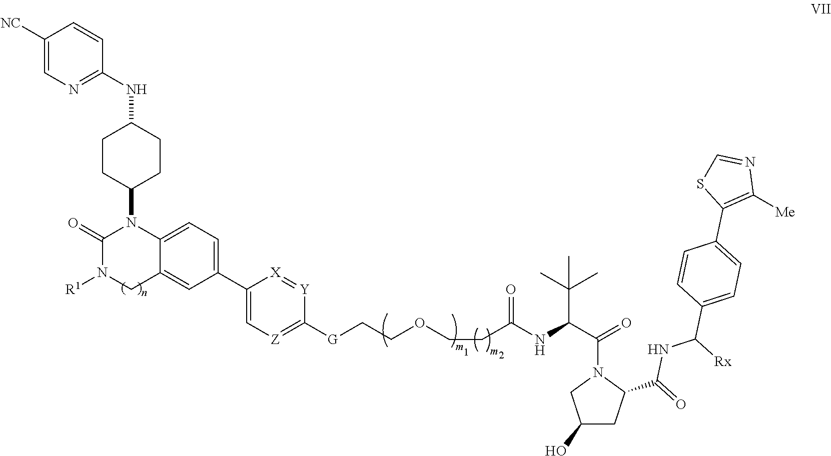

In Formula VII, m₁ is an integer from 0 to 6, m₂ is an integer from 0 to 15, G is absent, CH₂, NH, O, CONH, or NHCO; Rₓ is H or CH₃; X, Y, and Z are each independently CH or N; and the definitions of R¹ and n are the same as in Formula II; In one embodiment, G is O.

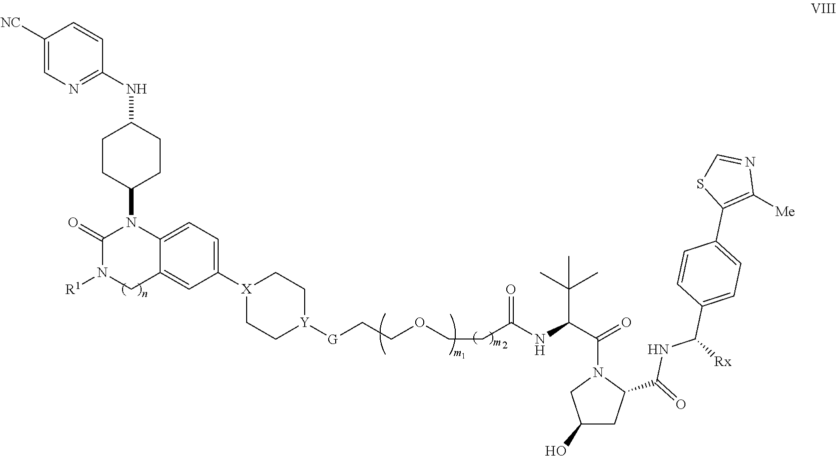

In Formula VIII, m₁ is an integer from 0 to 6, m₂ is an integer from 0 to 15, G is absent, CH₂, NH, O, CONH, or NHCO, Rₓ is H or CH₃, X and Y are each independently CH or N, and the definitions of R¹ and n are the same as in Formula II; In one embodiment, when Y is N, G is absent; When Y is CH, G is a heteroatom (such as NH or O).

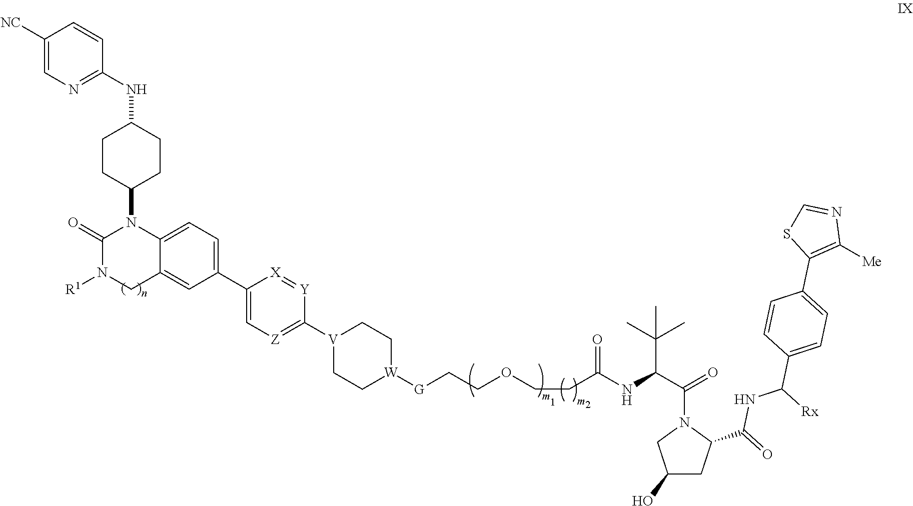

In Formula IX, m₁ is an integer from 0 to 6, m₂ is an integer from 0 to 15, G is absent, CH₂, NH, O, CONH, or NHCO; Rₓ is H or CH₃; X, Y, and Z are each independently CH or N, V and W are each independently CH or N, and the definitions of R¹ and n are the same as in Formula II; In one embodiment, when W is N, G is absent; When W is CH, G is a heteroatom (such as NH or O).

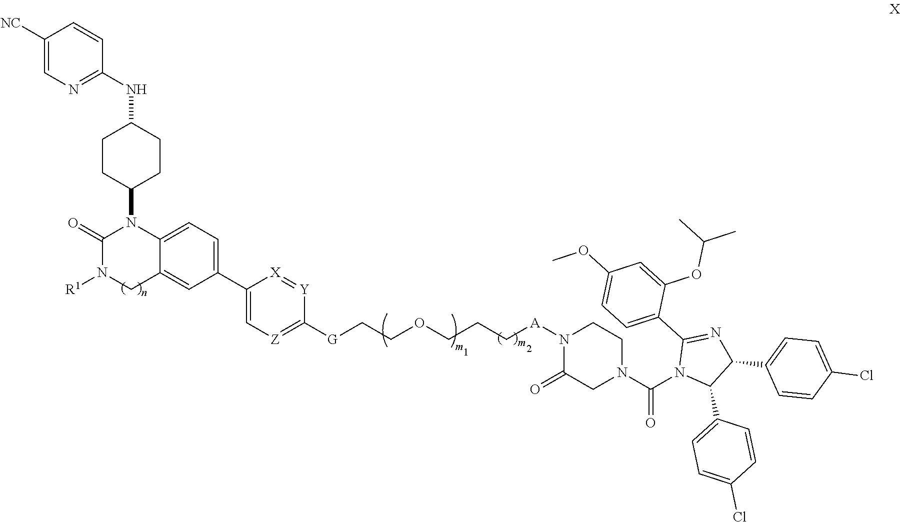

In Formula X, m₁ is an integer from 0 to 6, m₂ is an integer from 0 to 15, G is absent, CH₂, NH, O, CONH, or NHCO; A is CH₂ or CO; X, Y, and Z are each independently CH or N; and the definitions of R¹ and n are the same as in Formula II; In one embodiment, G is O.

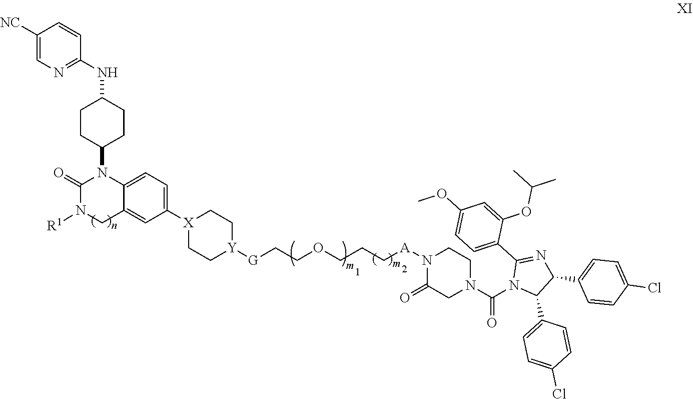

In Formula XI, m₁ is an integer from 0 to 6, m₂ is an integer from 0 to 15, G is absent, CH₂, NH, O, CONH, or NHCO; A is CH₂ or CO; X and Y are each independently CH or N; and the definitions of R¹ and n are the same as in Formula II; In one embodiment, when Y is N, G is absent; When Y is CH, G is a heteroatom (such as NH or O).

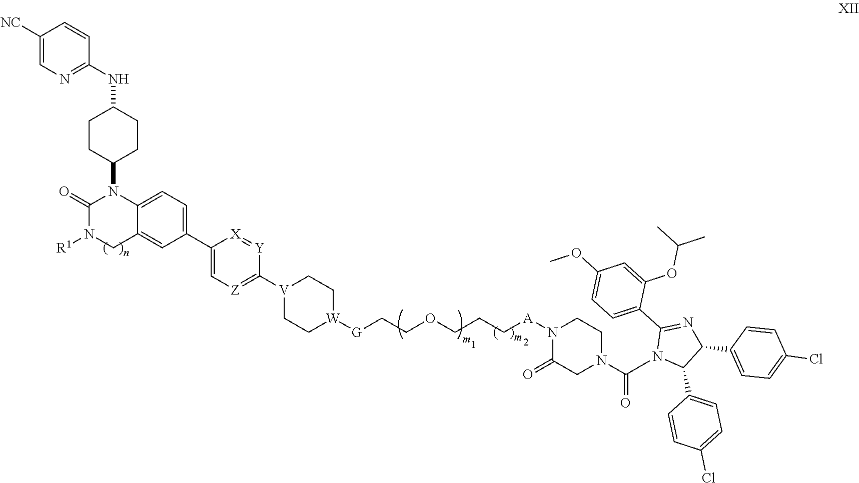

In Formula XII, m₁ is an integer from 0 to 6, m₂ is an integer from 0 to 15, G is absent, CH₂, NH, O, CONH, or NHCO; A is CH₂ or CO; X, Y, and Z are each independently CH or N, V and W are each independently CH or N, and the definitions of R¹ and n are the same as in Formula II; In one embodiment, when W is N, G is absent; When W is CH, G is a heteroatom (such as NH or O).

As a specific embodiment, the said small molecule conjugate has a structure as shown in Formula IX, wherein n=0; R¹ is benzyl; X is CH; at least one of Y and Z is N, and when they are not both N, the other is CH; G is absent; m₁ is 0; m₂ is 0, 1, or 2; and Rx is methyl.

More specifically, the said small molecule conjugate is a compound as shown in any one of the following formulae 1 to 138:

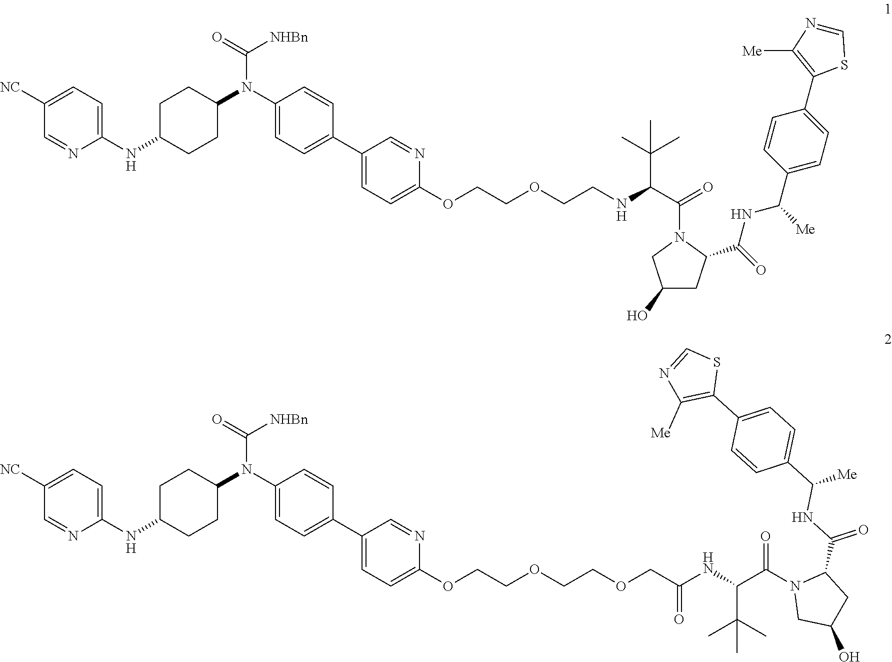

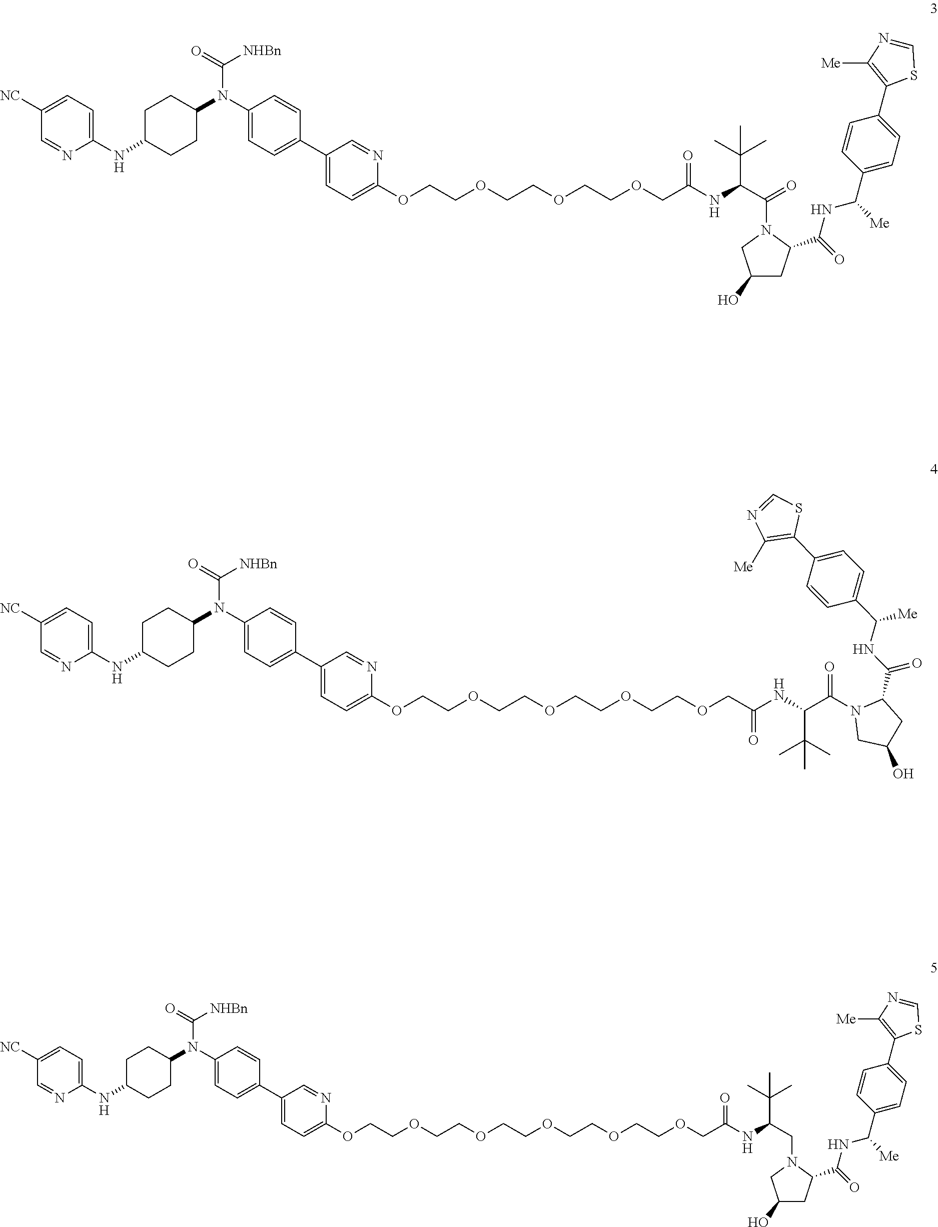

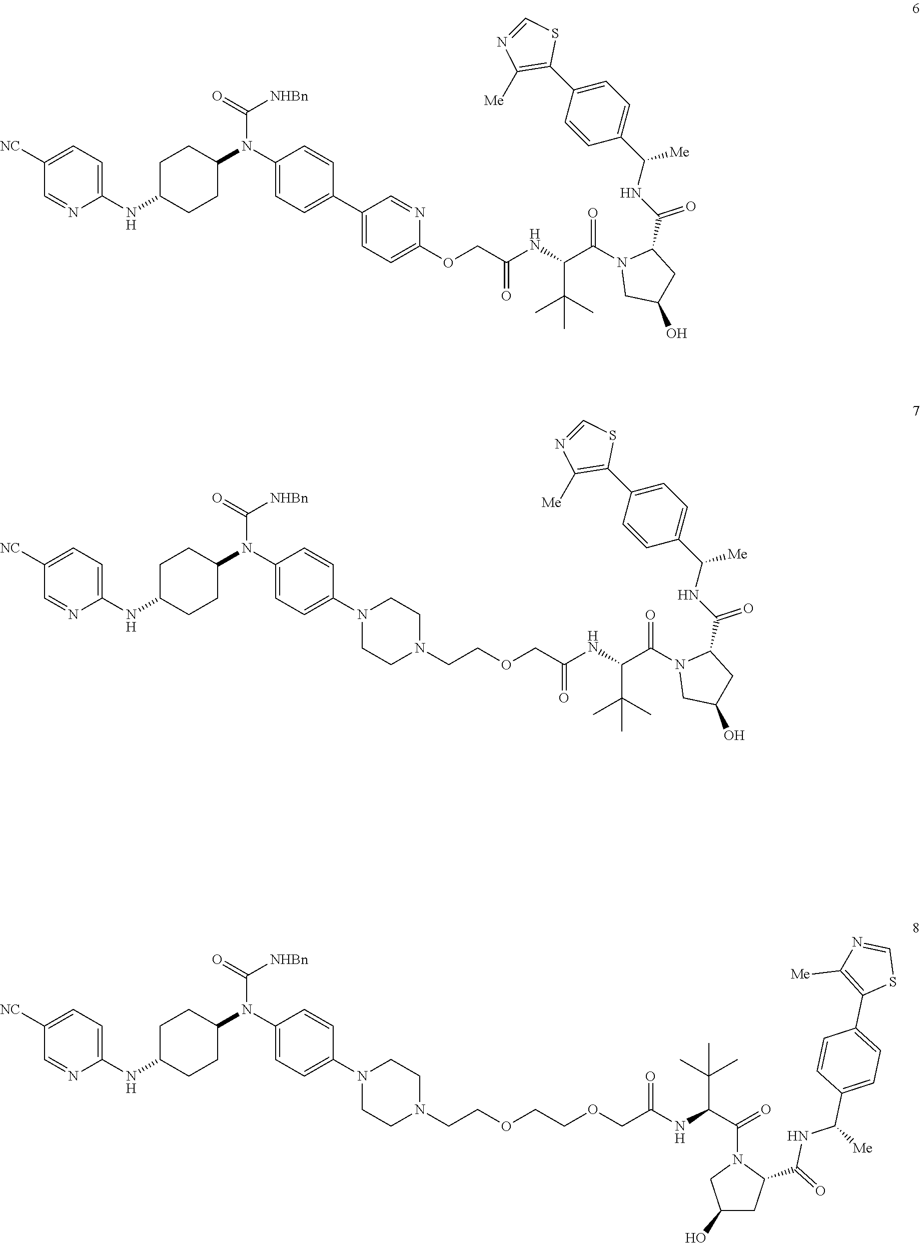

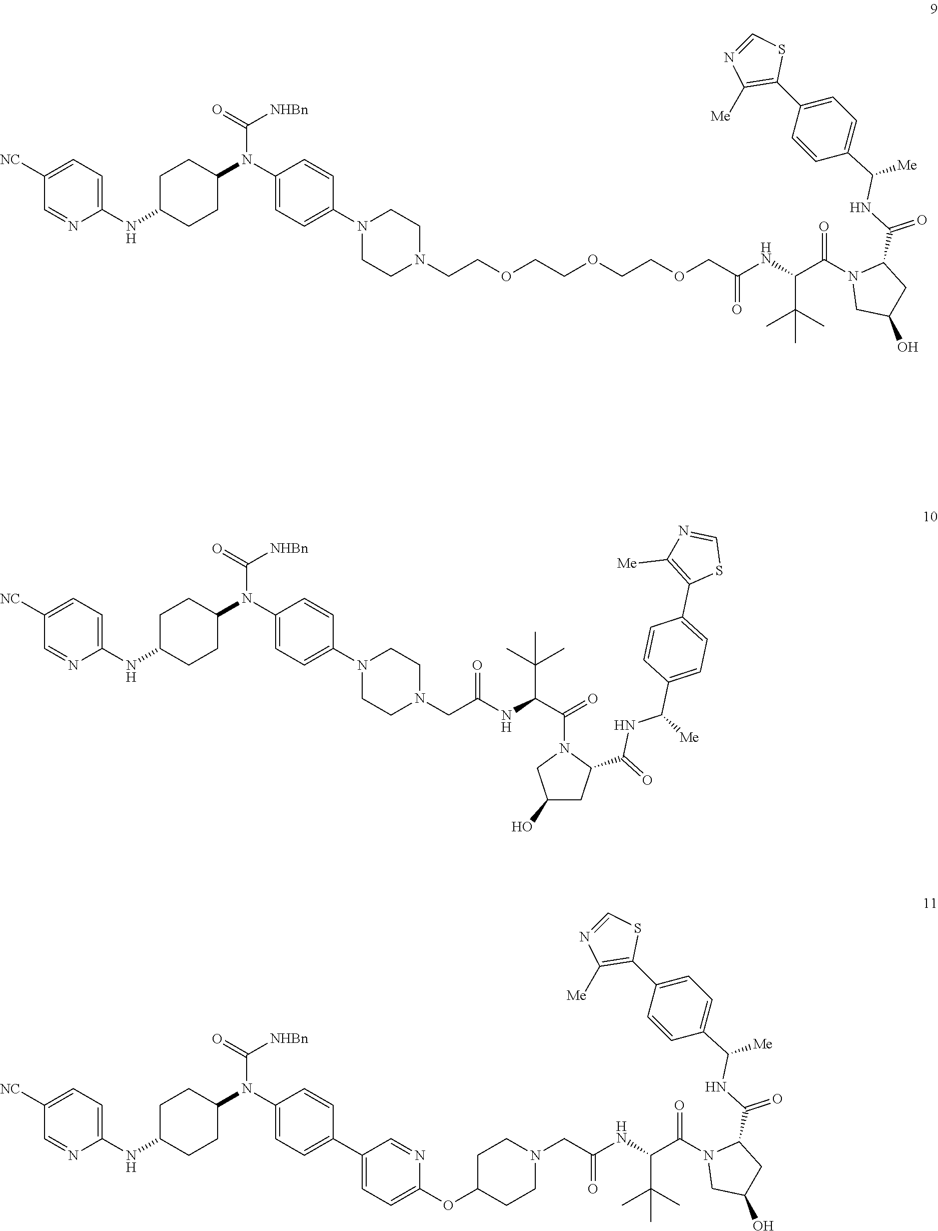

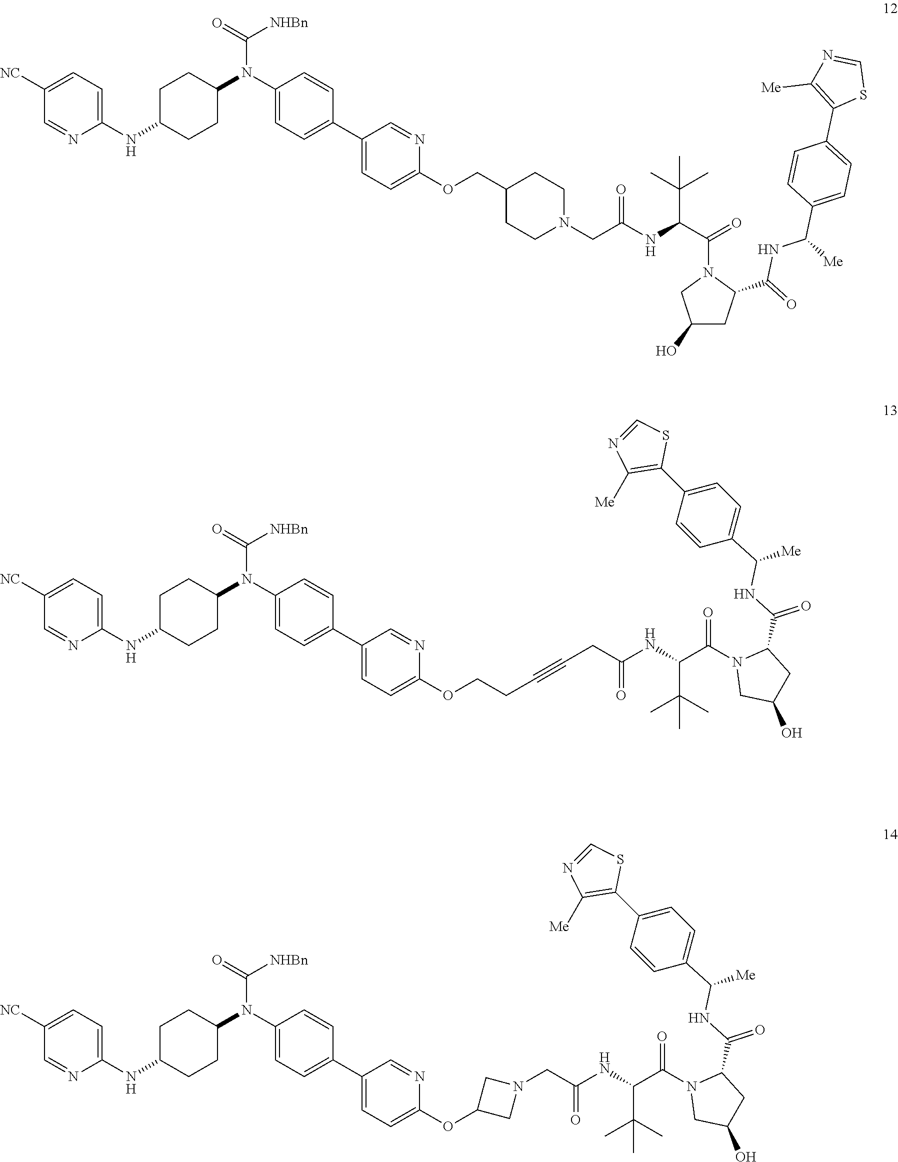

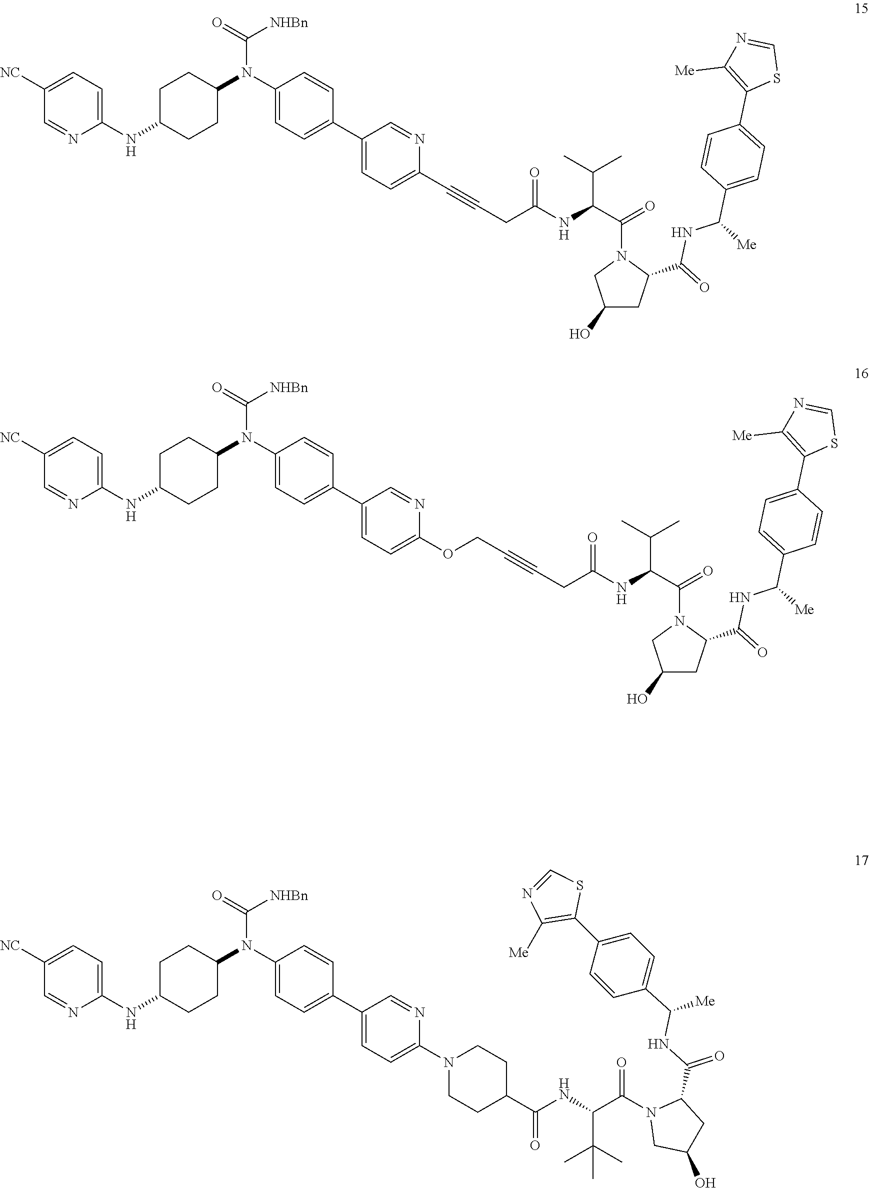

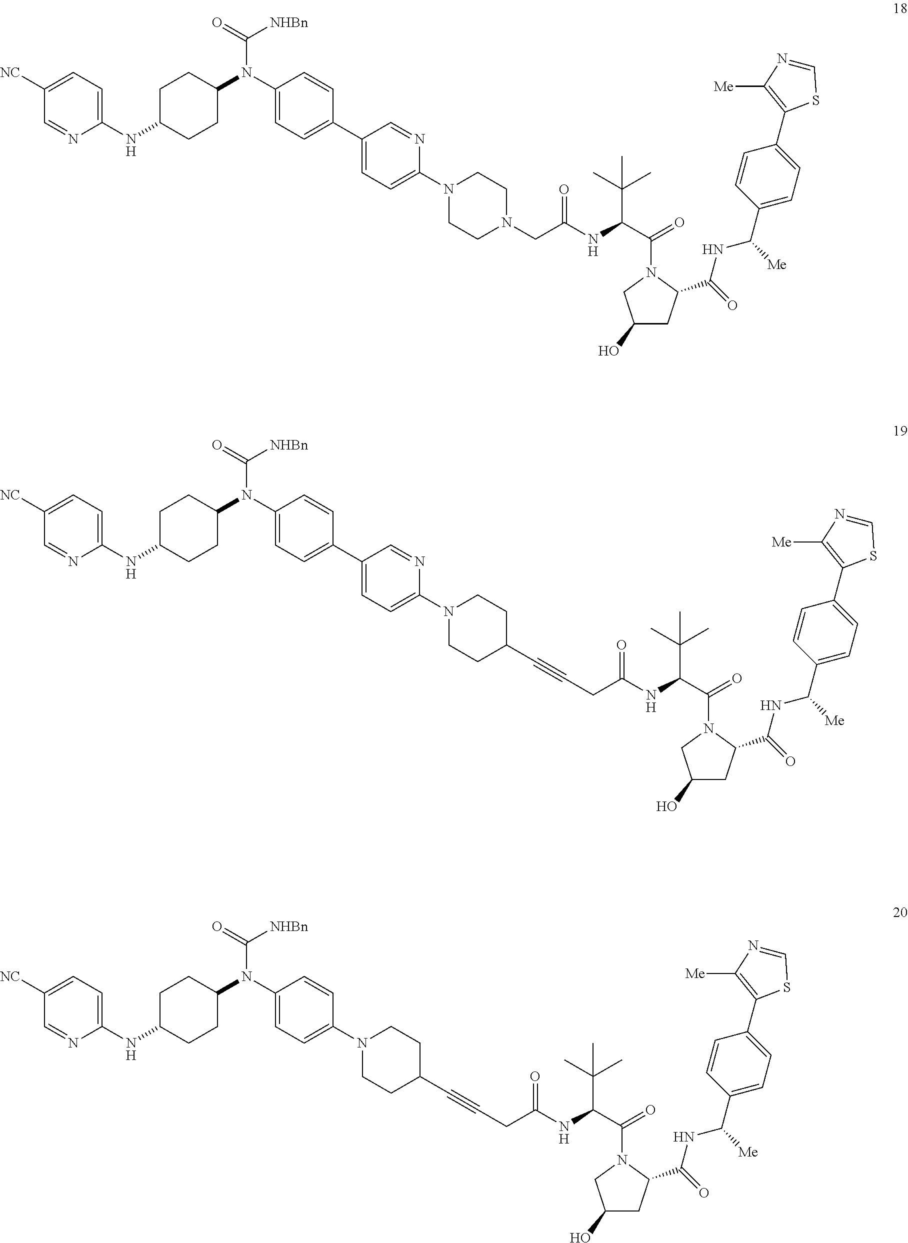

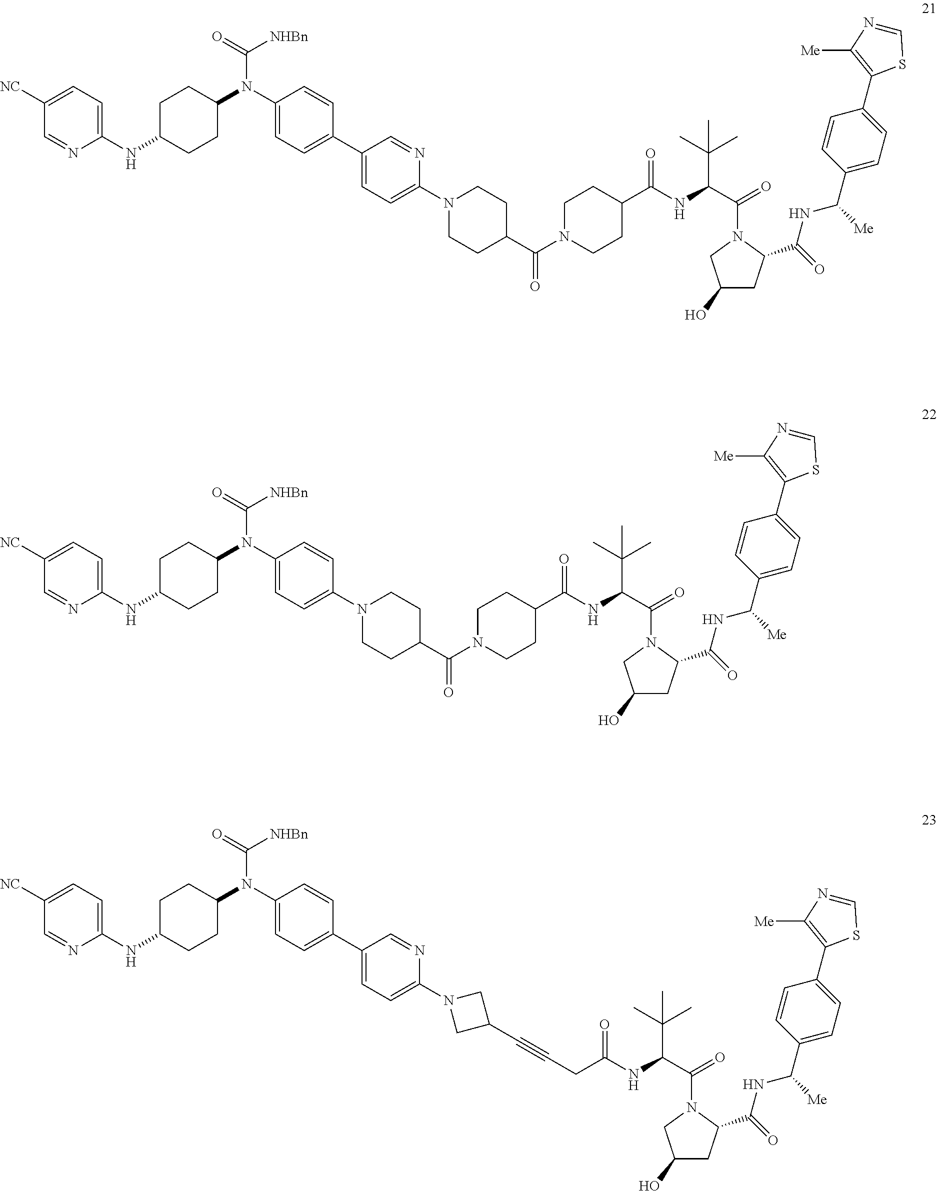

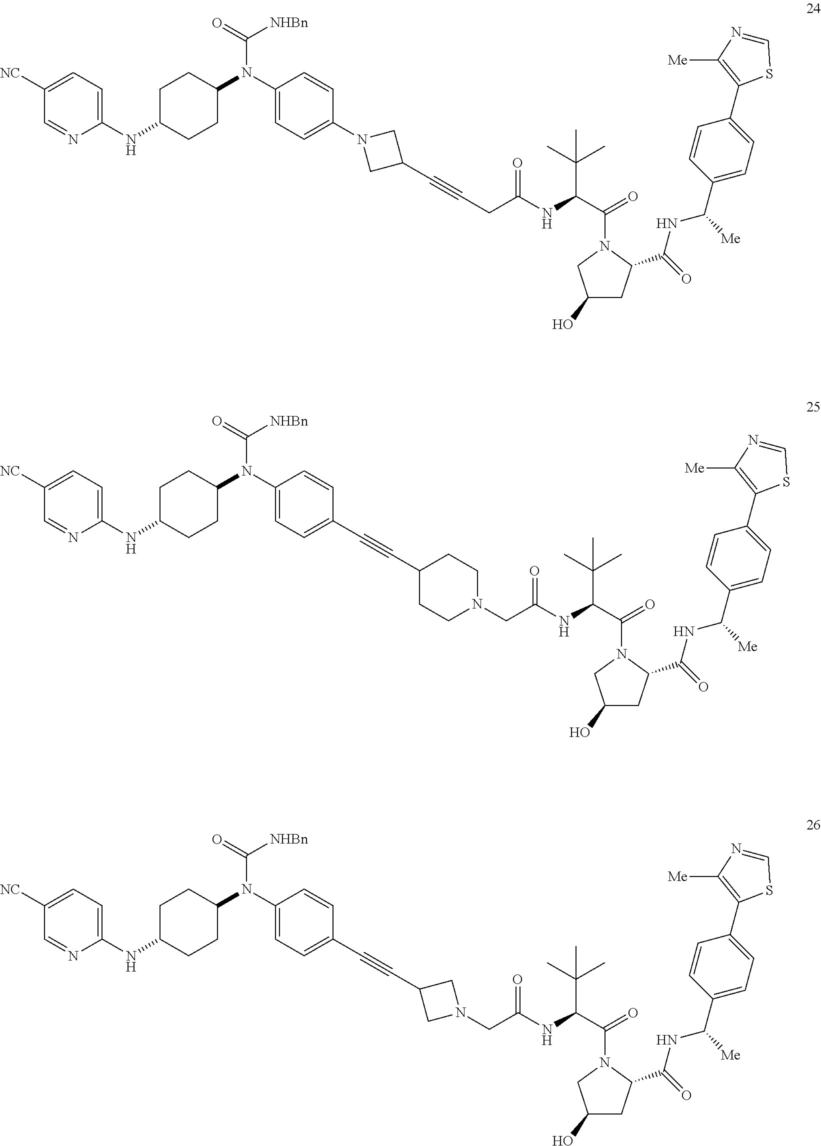

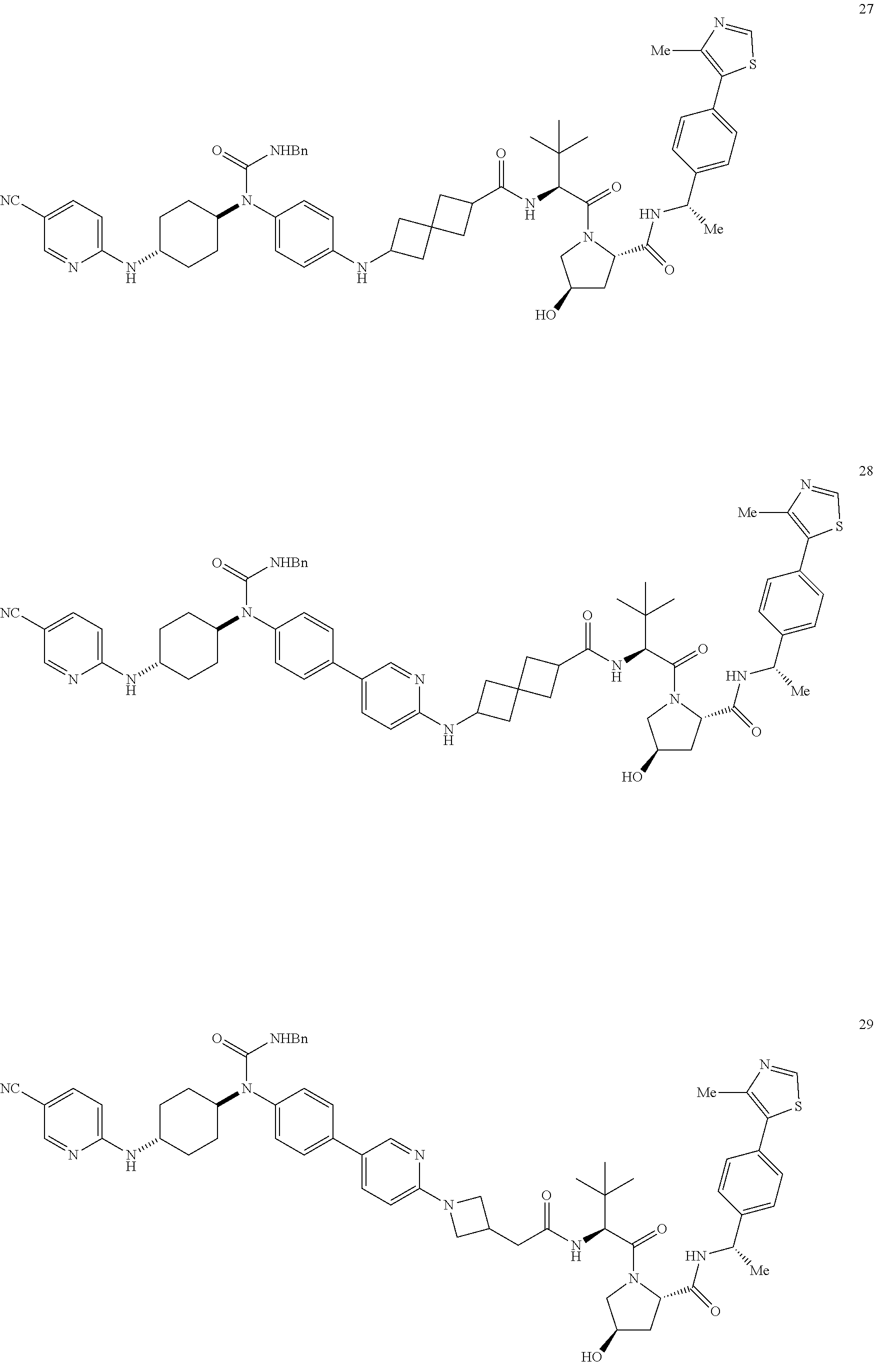

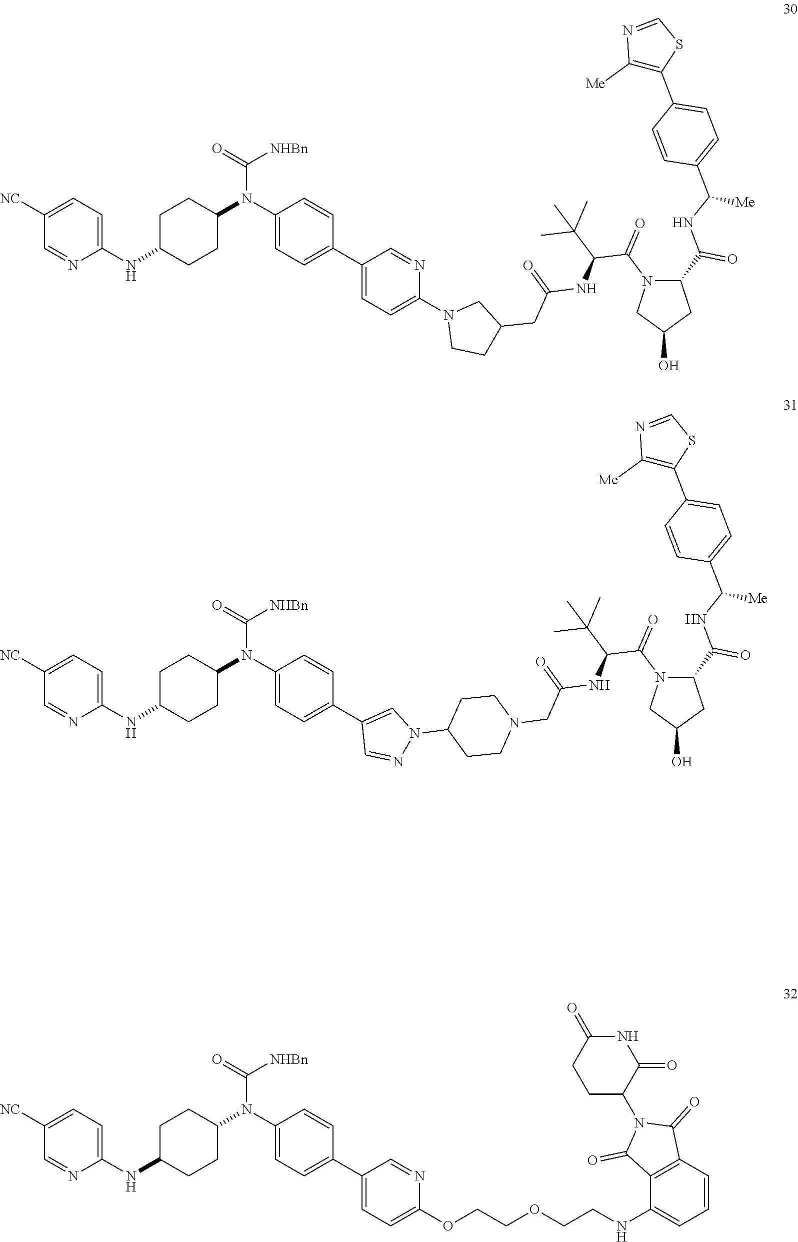

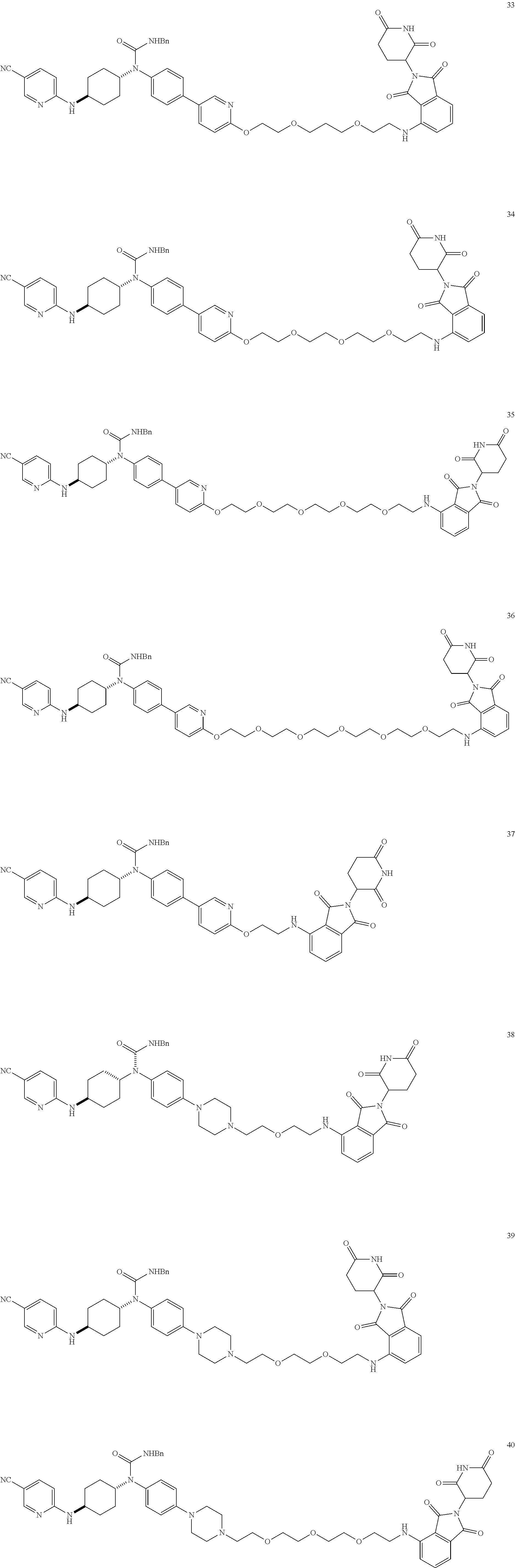

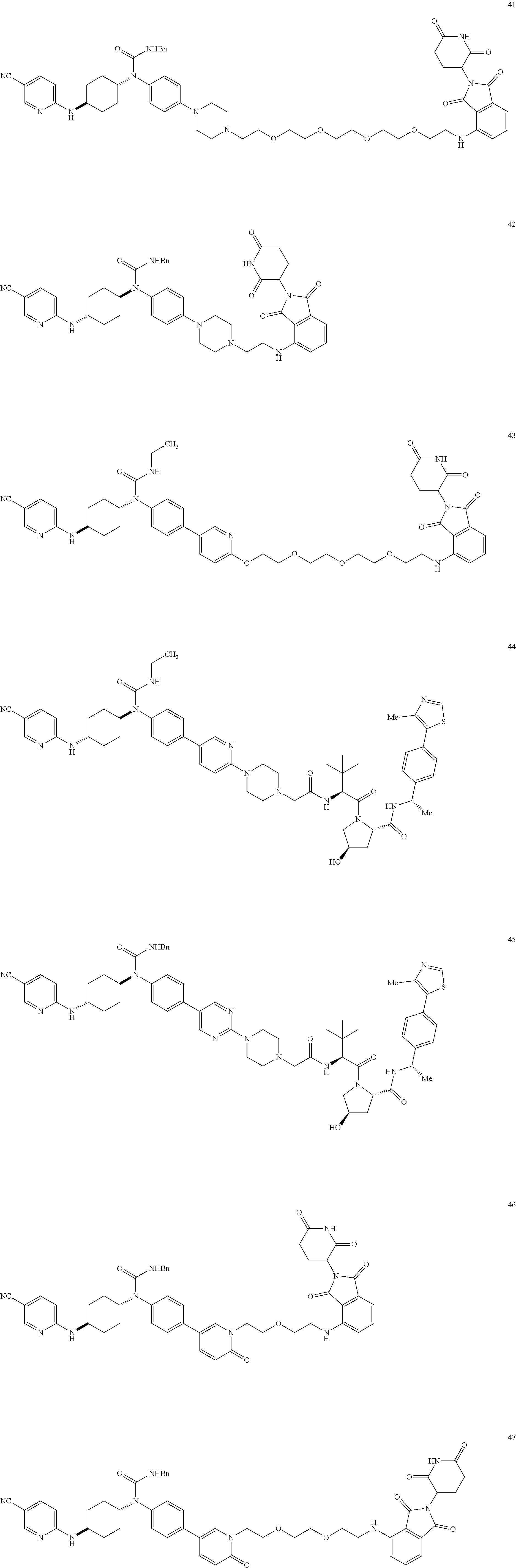

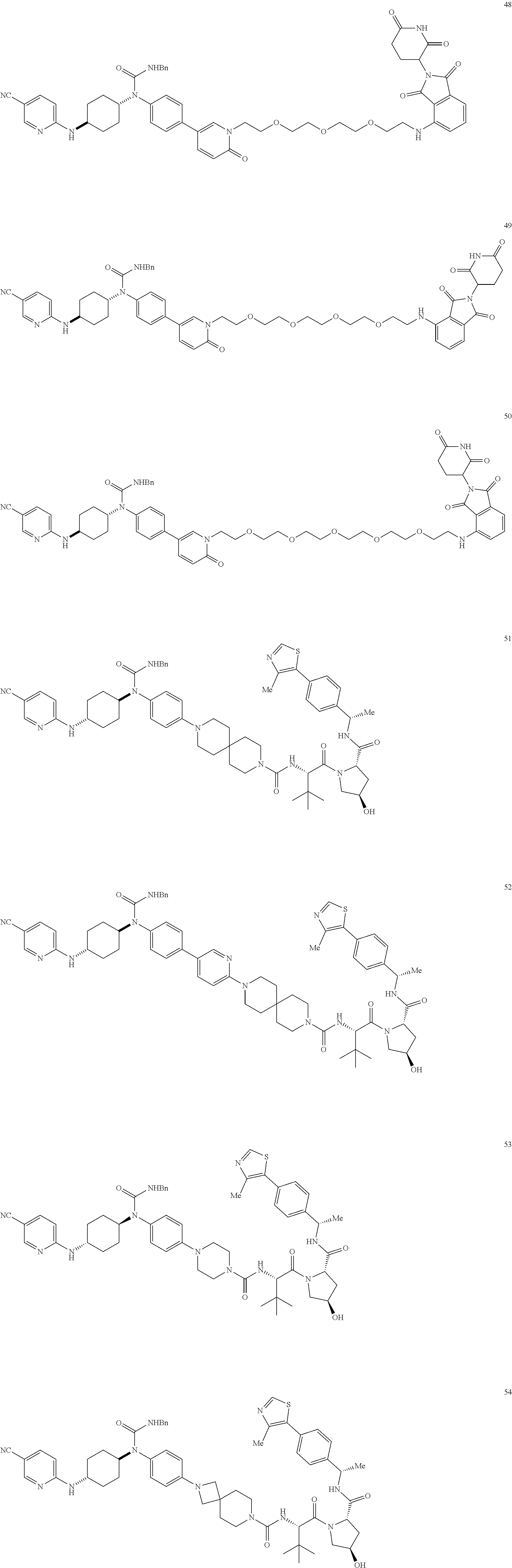

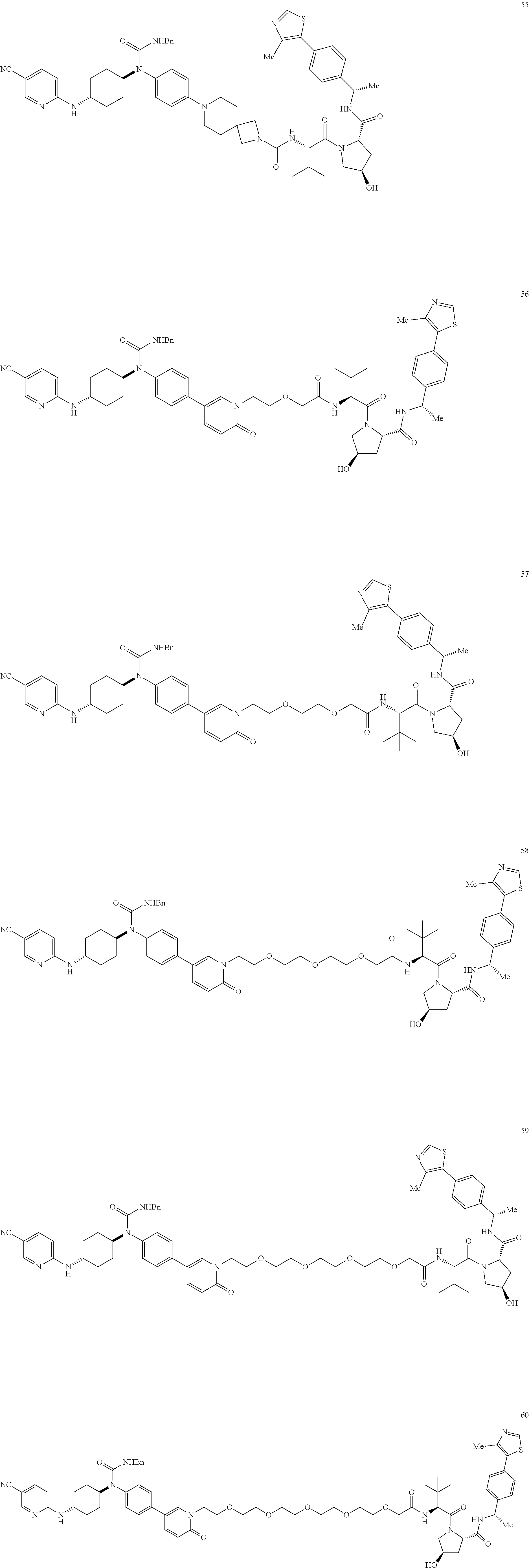

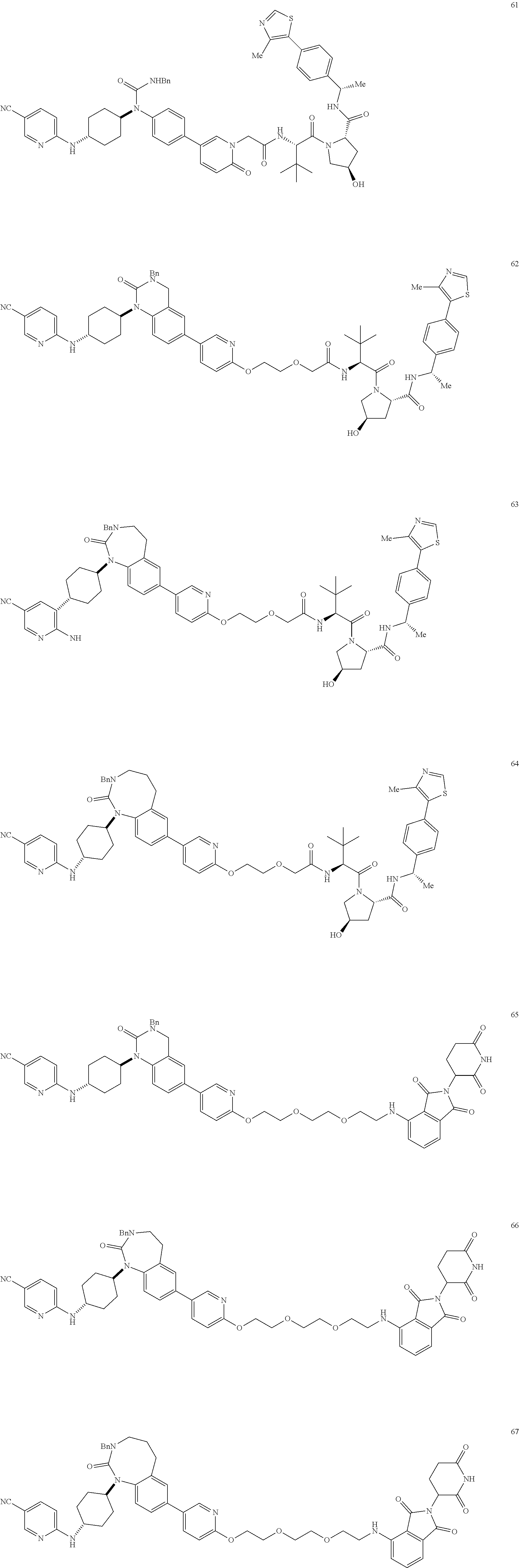

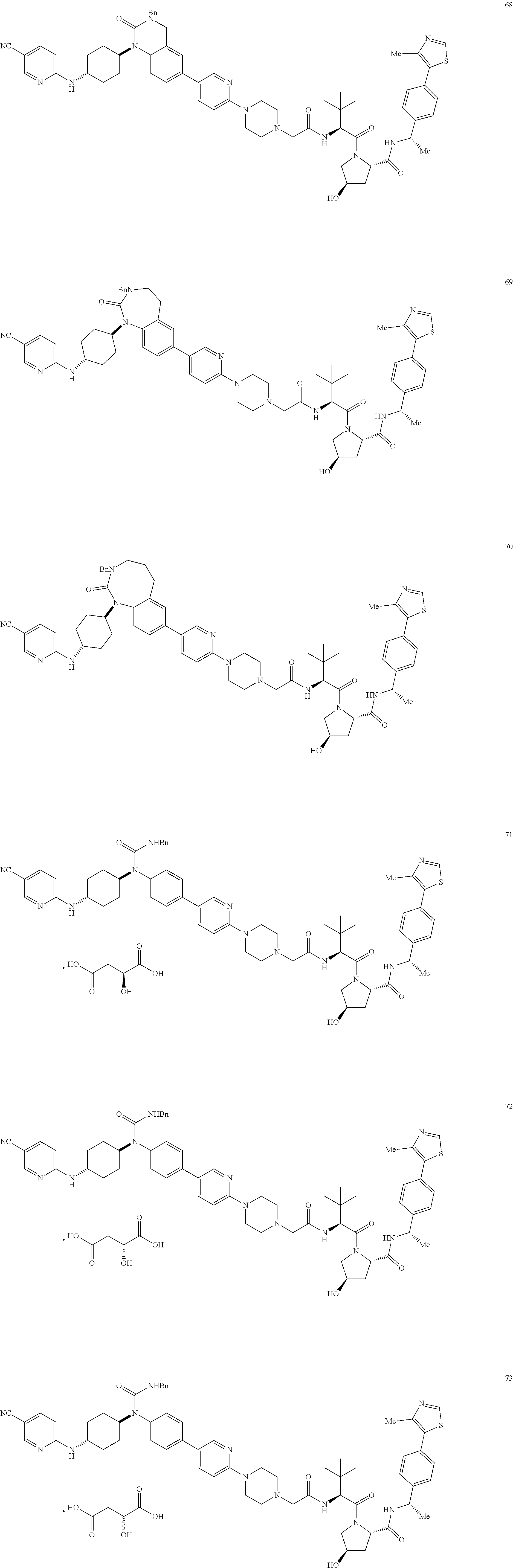

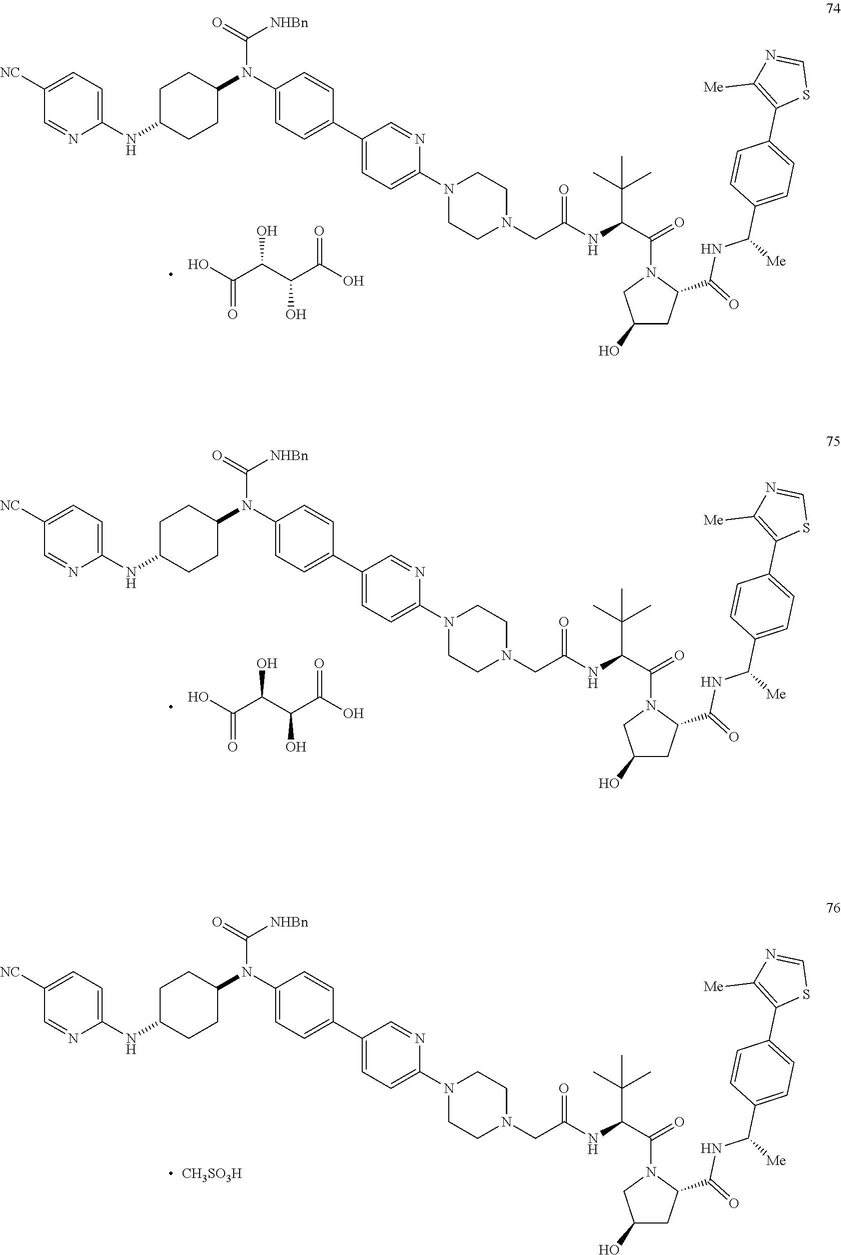

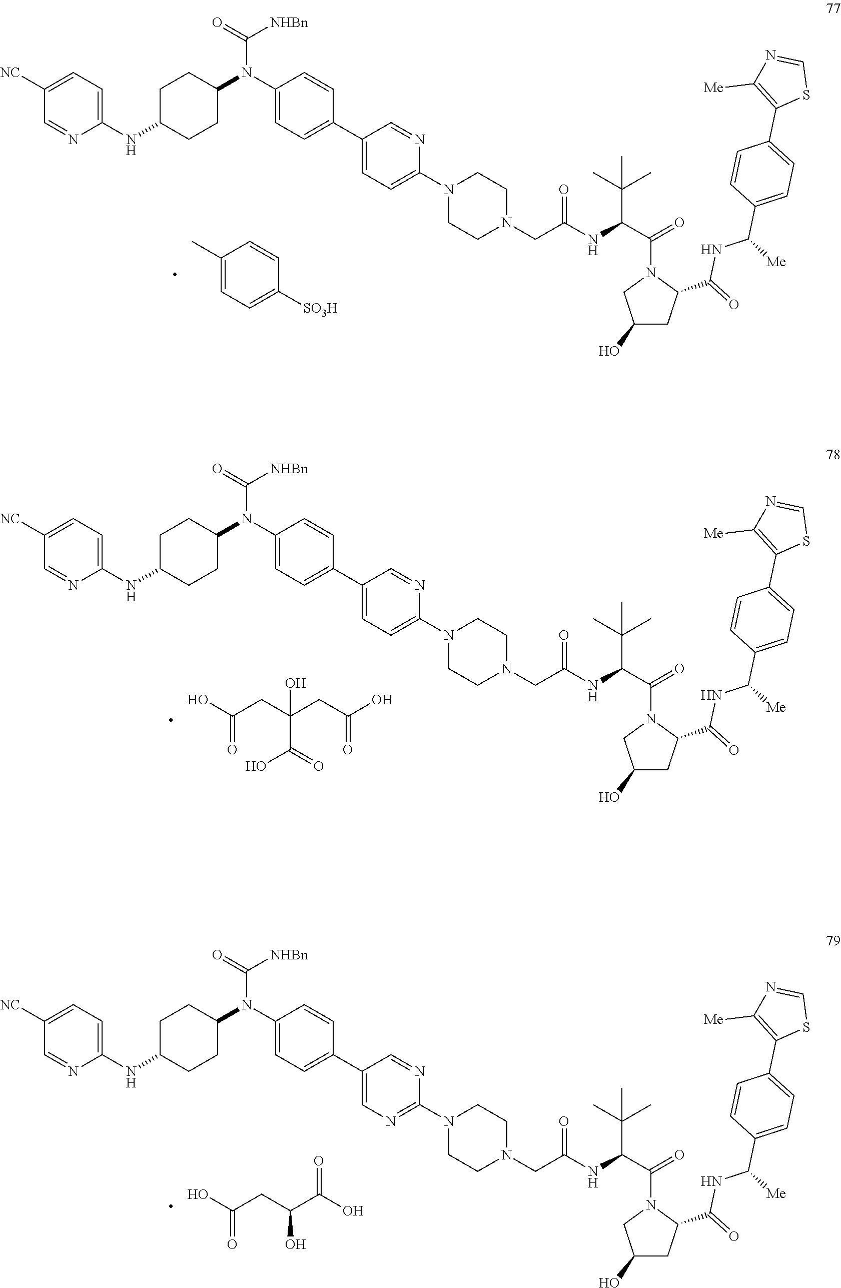

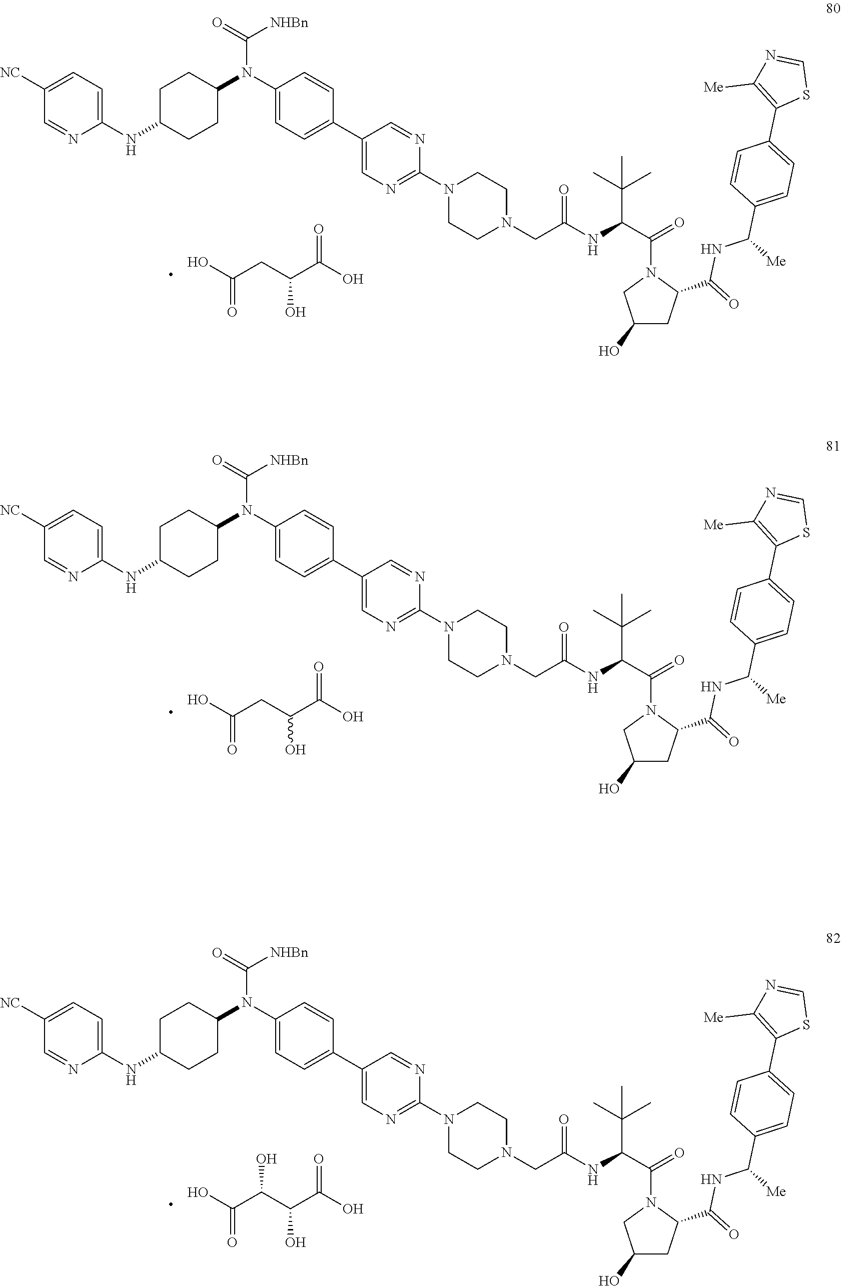

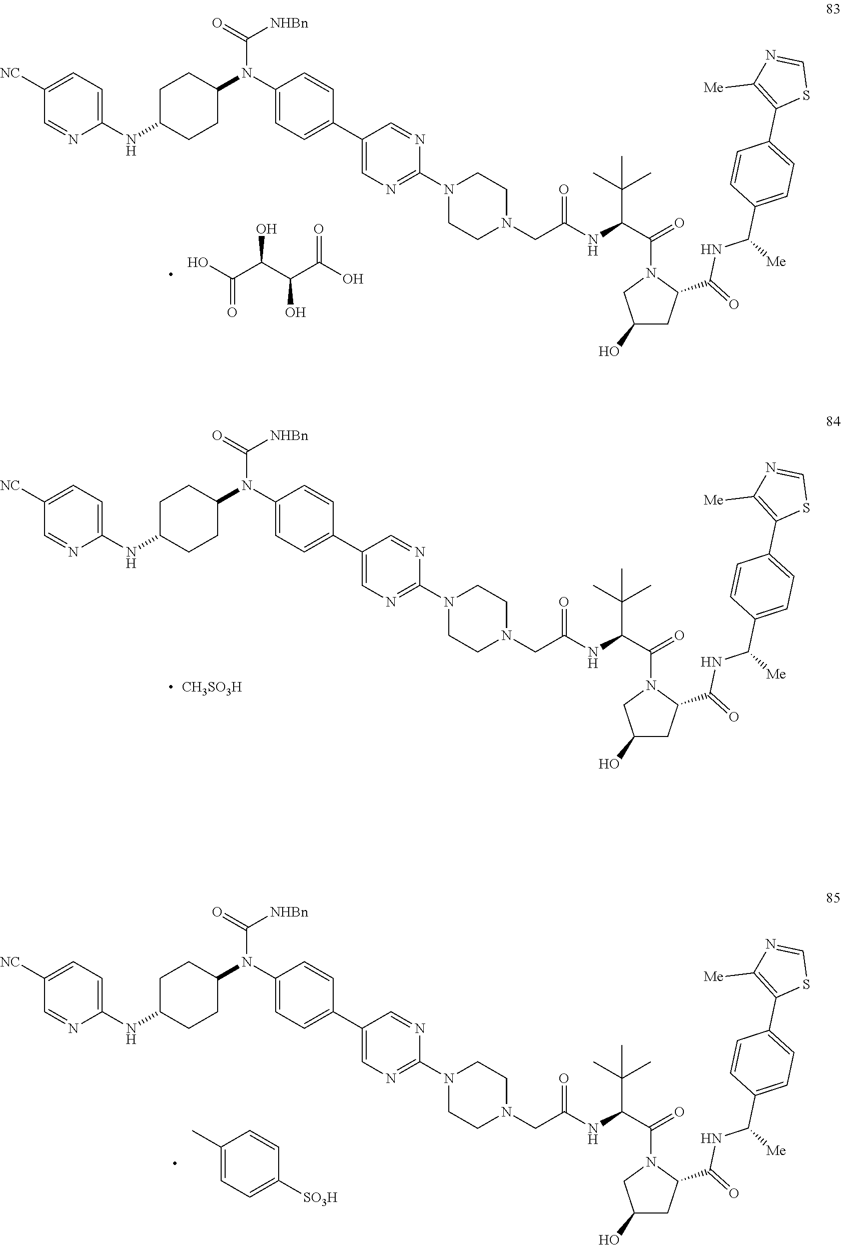

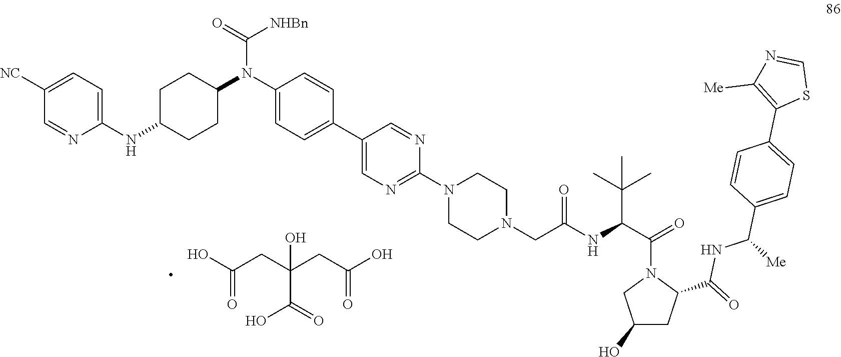

Another object of the present invention is to provide a method for preparing the small molecule conjugate as described in any one of the above technical solutions (preferably having any one of the structures as shown in Formula I, Formulae IV-XII, or Formulae 1-86).

Taking the structure shown in Formula I as an example, the compounds provided by the present invention as shown in Formula I (or any one of the structures shown in Formulae IV-XII, or Formulae 1-86) can be synthesized by an amide condensation reaction, a substitution reaction, a click reaction, or a cross-coupling reaction, etc., between a corresponding E3 ligase ligand (e.g., Pomalidomide-type derivatives, VHL ligand-type derivatives, and MDM2 ligand-type derivatives) and a corresponding T-part derivative.

Specifically, the method for preparing the small molecule conjugate as shown in Formula IV (taking G as 0, A as NH, X as CH, Y as N, and Z as CH as an example) comprises the following steps: the small molecule conjugate according to Formula IV is obtained by a nucleophilic substitution reaction between a CDK12/13 ligand terminal derivative (as shown in Formula a) and a Pomalidomide terminal derivative (as shown in Formula b);
wherein, in Formula a, the definitions of R¹, m₁, m₂, and n are the same as in Formula IV, and in Formula b, the definition of B is the same as in Formula IV.

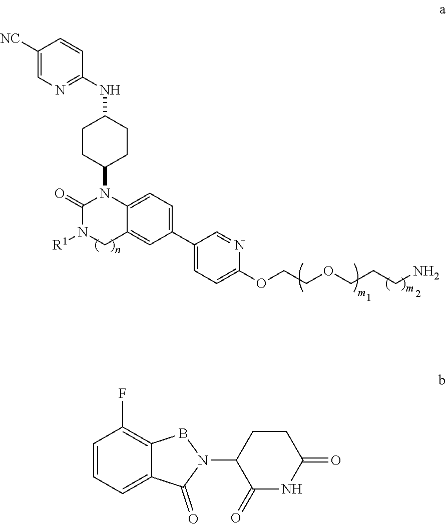

The method for preparing the small molecule conjugate as shown in Formula V (where G is CH₂, A is NH, X is N, and Y is N) comprises the following steps:

The small molecule conjugate according to Formula V is obtained by a nucleophilic substitution reaction between a CDK12/13 ligand terminal derivative (as shown in Formula c) and a Pomalidomide terminal derivative (as shown in Formula b);
wherein, in Formula c, the definitions of R¹, m₁, m₂, and n are the same as in Formula V.

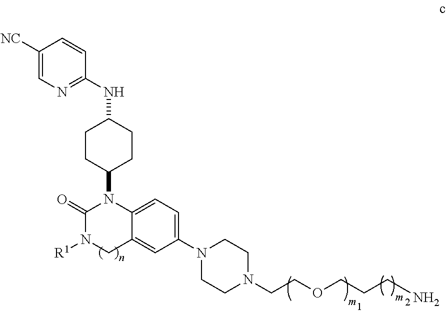

The method for preparing the small molecule conjugate as shown in Formula VI (where G is CH₂, A is NH, X is CH, Y is N, Z is CH, V is N, and W is N) comprises the following steps:
The small molecule conjugate according to Formula VI is obtained by a nucleophilic substitution reaction between a CDK12/13 ligand terminal derivative (as shown in Formula d) and a Pomalidomide terminal derivative (as shown in Formula b);
wherein, in Formula d, the definitions of R¹, m₁, m₂, and n are the same as in Formula VI.

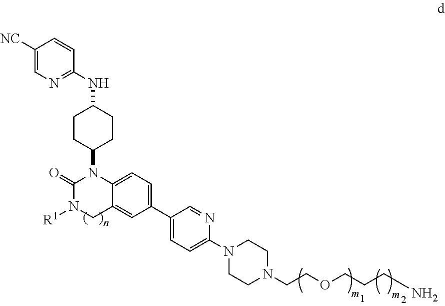

The method for preparing the small molecule conjugate as shown in Formula VII (where G is O, X is CH, Y is N, and Z is CH) comprises the following steps:
The small molecule conjugate according to Formula VII is obtained by a condensation reaction between a CDK12/13 ligand terminal derivative (as shown in Formula e) and a VHL ligand derivative (as shown in Formula f);
wherein, in Formula e, the definitions of R¹, m₁, m₂, and n are the same as in Formula VII.

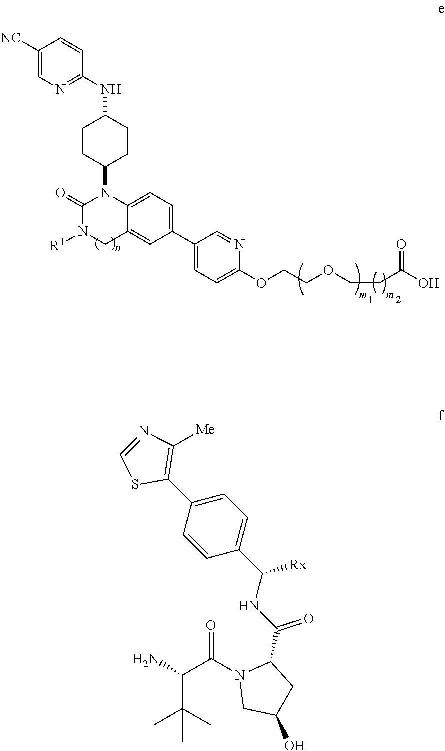

The method for preparing the small molecule conjugate as shown in Formula VIII (where G is CH₂, X is N, and Y is N) comprises the following steps:
The small molecule conjugate according to Formula VIII is obtained by a condensation reaction between a CDK12/13 ligand terminal derivative (as shown in Formula g) and a VHL ligand derivative (as shown in Formula f);
wherein, in Formula g, the definitions of R¹, m₁, m₂, and n are the same as in Formula VIII, and in Formula f, the definition of Rx is the same as in Formula VIII.

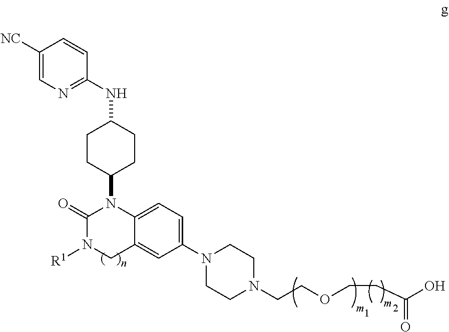

The method for preparing the small molecule conjugate as shown in Formula IX (where G is absent, X is CH, Y is N, Z is CH, V is N, and W is N) comprises the following steps:
The small molecule conjugate according to Formula X is obtained by a condensation reaction between a CDK12/13 ligand terminal derivative (as shown in Formula h) and a VHL ligand derivative (as shown in Formula f);
wherein, in Formula h, the definitions of R¹, m₁, m₂, and n are the same as in Formula IX, and in Formula f, the definition of Rx is the same as in Formula IX.

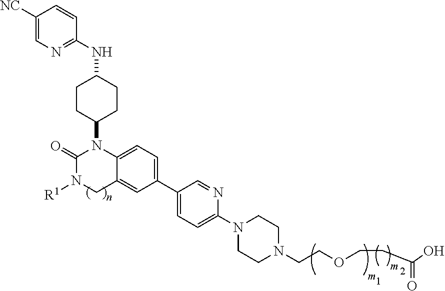

The method for preparing the small molecule conjugate as shown in Formula X (where G is O, X is CH, Y is N, Z is CH, and A is CO) comprises the following steps:
The small molecule conjugate according to Formula X is obtained by a condensation reaction between a CDK12/13 ligand terminal derivative (as shown in Formula e) and an MDM2 ligand derivative (as shown in Formula j);
wherein, in the said small molecule conjugate according to Formula X, in Formula e, the definitions of R¹, m₁, m₂, and n are the same as in Formula X.

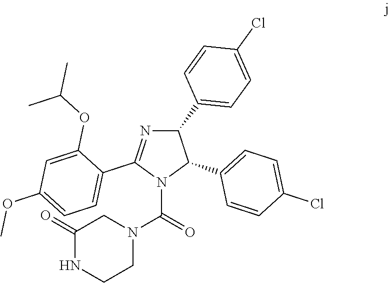 [SMILES: COc1ccc(C2=N[C@H](c3ccc(Cl)cc3)[C@H](c3ccc(Cl)cc3)N2C(=O)N2CCNC(=O)C2)c(OC(C)C)c1]

The method for preparing the small molecule conjugate as shown in Formula XI (where G is CH₂, X is N, Y is N, and A is CO) comprises the following steps: the small molecule conjugate according to Formula XI is obtained by a condensation reaction between a CDK12/13 ligand terminal derivative (as shown in Formula g) and an MDM2 ligand derivative (as shown in Formula j); wherein, in Formula g, the definitions of R¹, m₁, m₂, and n are the same as in Formula XI.

The method for preparing the small molecule conjugate as shown in Formula XII (where G is CH₂, X is CH, Y is N, Z is CH, V is N, W is N, and A is CO) comprises the following steps: the small molecule conjugate according to Formula XII is obtained by a condensation reaction between a CDK12/13 ligand terminal derivative (as shown in Formula h) and an MDM2 ligand derivative (as shown in Formula j); wherein, in Formula h, the definitions of R¹, m₁, m₂, and n are the same as in Formula XII.

The above CDK12/13 ligand terminal derivatives (as shown in Formulae a, c, d, e, g, and h) also fall within the scope of protection of the present invention. The specific synthesis process for the CDK12/13 ligand terminal derivatives (as shown in Formulae a, c, d, e, g, and h) is as follows:
When n in Formula II is 0: the compounds corresponding to Formulae a, c, d, e, g, and h are the structures as shown in Formulae a0, c0, d0, e0, g0, and h0:

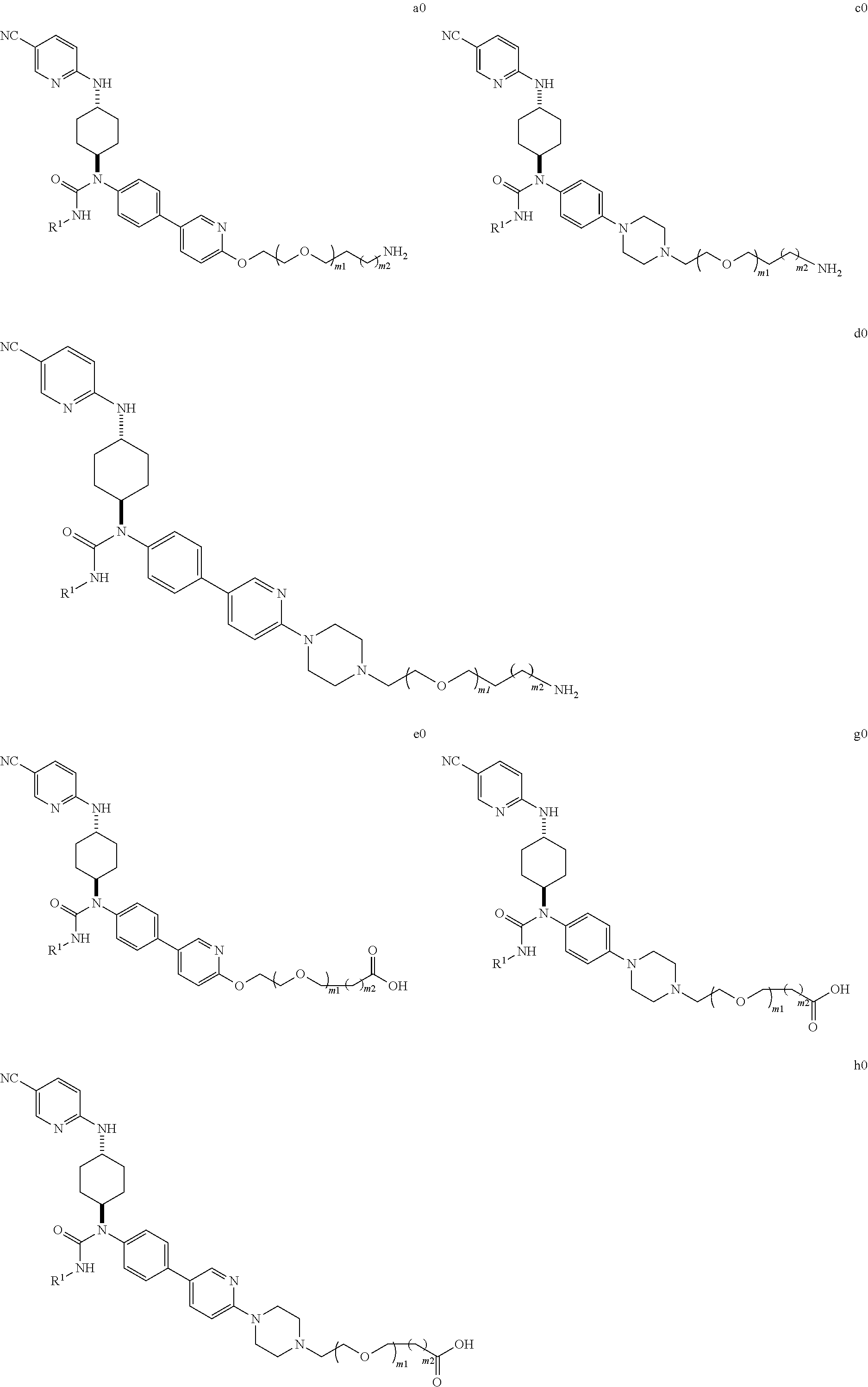

1) CDK12/13 Ligand Terminal Derivative (as shown in Formula a0): 1. Coupling the compound as shown in Formula 1a with bis(pinacolato)diboron to obtain the compound as shown in Formula 2a; 2. Subjecting 5-bromo-2-fluoropyridine (Formula 3a) and the compound as shown in Formula 4a to a nucleophilic reaction to obtain the compound as shown in Formula 5a; 3. Coupling the compound as shown in Formula 2a with the compound as shown in Formula 5a and then removing the protecting group to obtain the compound as shown in Formula a0.

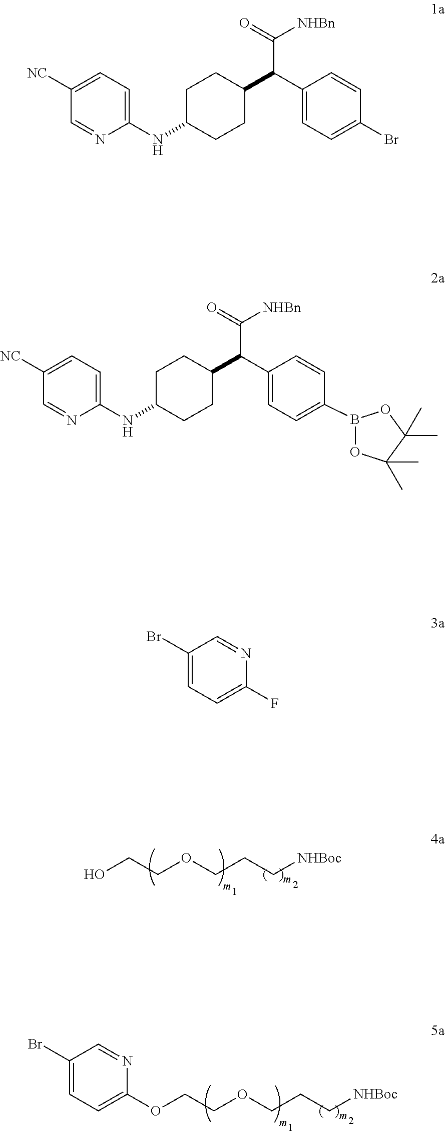

The preparation method for the compound as shown in Formula 1a is as follows: Coupling 1-bromo-4-iodobenzene (Formula 1aa) with trans-(4-aminocyclohexyl)carbamic acid tert-butyl ester (Formula 2aa) through a coupling reaction to obtain ((1R,4R)-4-((4-bromophenyl)amino)cyclohexyl)carbamic acid tert-butyl ester (Formula 3aa); subjecting the compound as shown in Formula 3aa and benzyl isocyanate (Formula 4aa) to a nucleophilic reaction followed by deprotection to obtain 1-((1R,4R)-4-aminocyclohexyl)-3-benzyl-1-(4-bromophenyl)urea (Formula 5aa); and then subjecting the compound as shown in Formula 5aa and 6-fluoronicotinonitrile (Formula 6aa) to a nucleophilic reaction to obtain the compound as shown in Formula 1a.

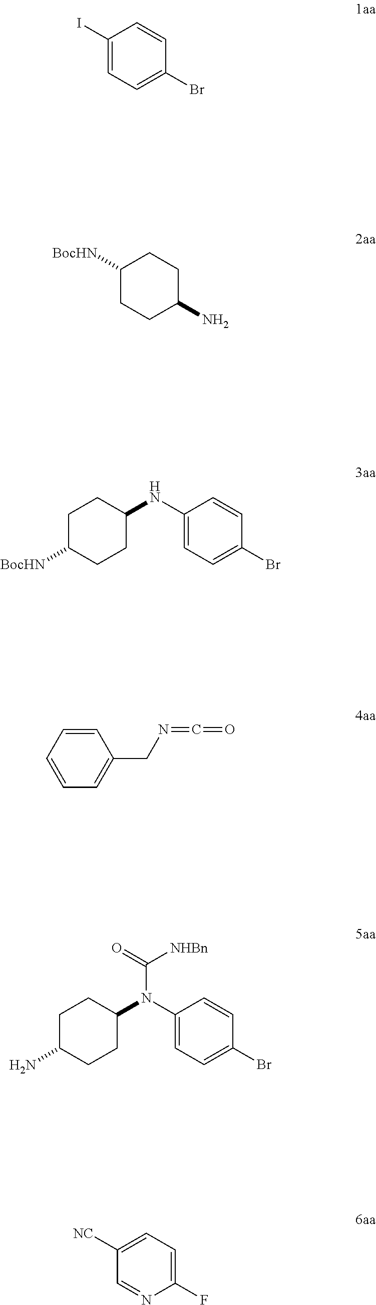

1) CDK12/13 Ligand Terminal Derivative (as shown in Formula c0): 1. Coupling Formula 1a with tert-butyl 1-piperazinecarboxylate (Formula 1c) and deprotecting to obtain Formula 3c; 2. Subjecting the compound as shown in Formula 2c and the compound as shown in Formula 3c to a nucleophilic substitution reaction and removing the protecting group to obtain the compound as shown in Formula c0.

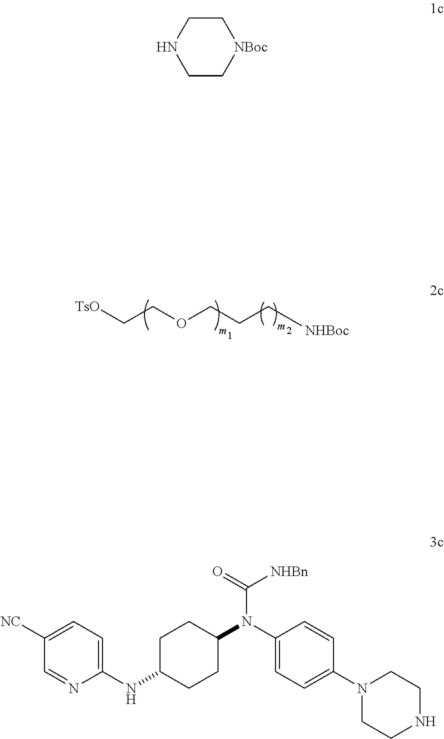

1) CDK12/13 Ligand Terminal Derivative (as shown in Formula d0): 1. Subjecting the compound as shown in Formula 3a and the compound as shown in Formula 1c to a nucleophilic substitution reaction to obtain the compound as shown in Formula 1d; 2. Coupling the compound of Formula 1d with the compound of Formula 2a and removing the protecting group to obtain the compound of Formula 2d; 3. Subjecting the compound as shown in Formula 2c and the compound as shown in Formula 2d to a nucleophilic substitution reaction and removing the protecting group to obtain the compound as shown in Formula d0.

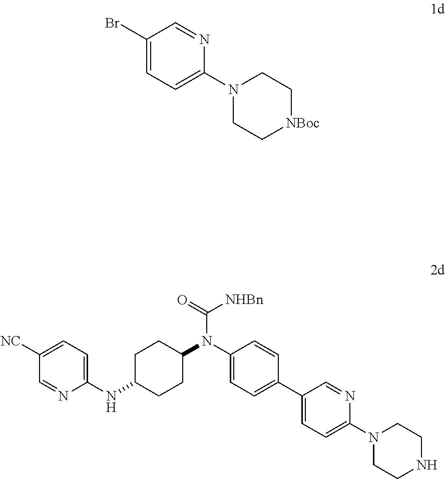

1) CDK12/13 Ligand Terminal Derivative (as shown in Formula e0): 1. Subjecting the compound as shown in Formula e1 and the compound as shown in Formula 3a to a nucleophilic substitution reaction to obtain e2; 2. Coupling the compound as shown in Formula e2 with the compound of Formula 2a and removing the protecting group to obtain the compound as shown in Formula e0.

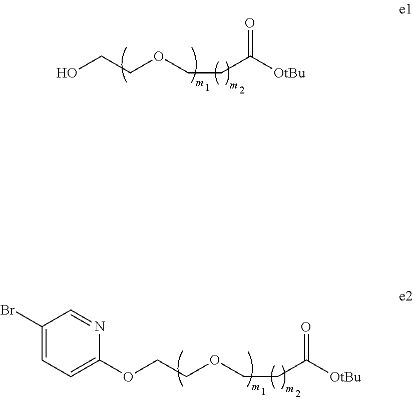

2) CDK12/13 Ligand Terminal Derivative (as shown in Formula g0): Subjecting the compound as shown in Formula 3c and the compound as shown in Formula g1 to a nucleophilic substitution reaction and removing the protecting group to obtain the compound as shown in Formula g0.

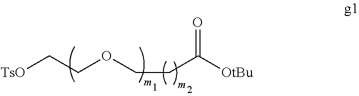

3) CDK12/13 Ligand Terminal Derivative (as shown in Formula h0): Subjecting the compound as shown in Formula 2d and the compound as shown in Formula g1 to a nucleophilic substitution reaction and removing the protecting group to obtain the compound as shown in Formula h0.

When n in Formula II is not 0 (taking n as 1 as an example): the compounds corresponding to Formulae a, c, d, e, g, and h are the structures as shown in Formulae ab1, cb1, db1, eb1, gb1, and hb1:

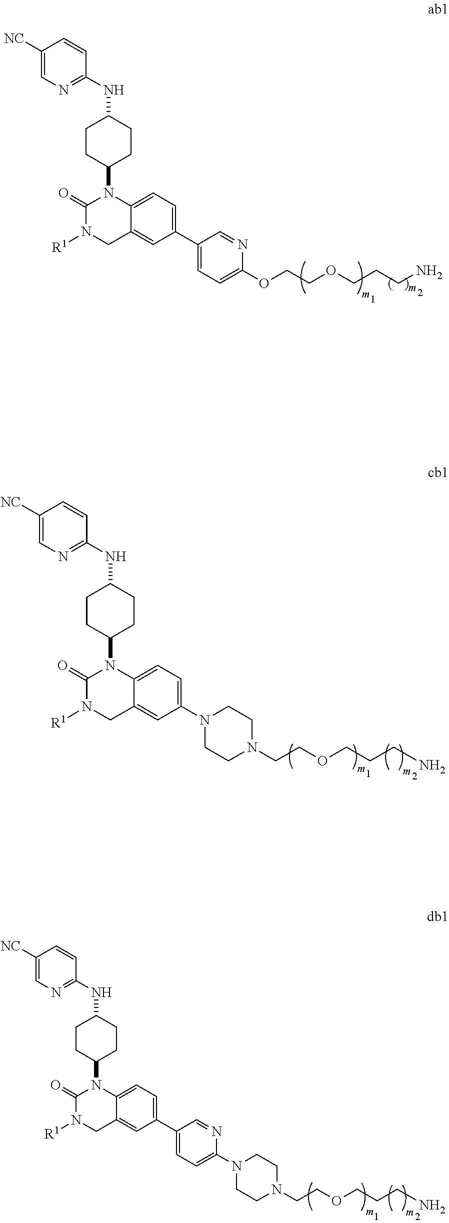

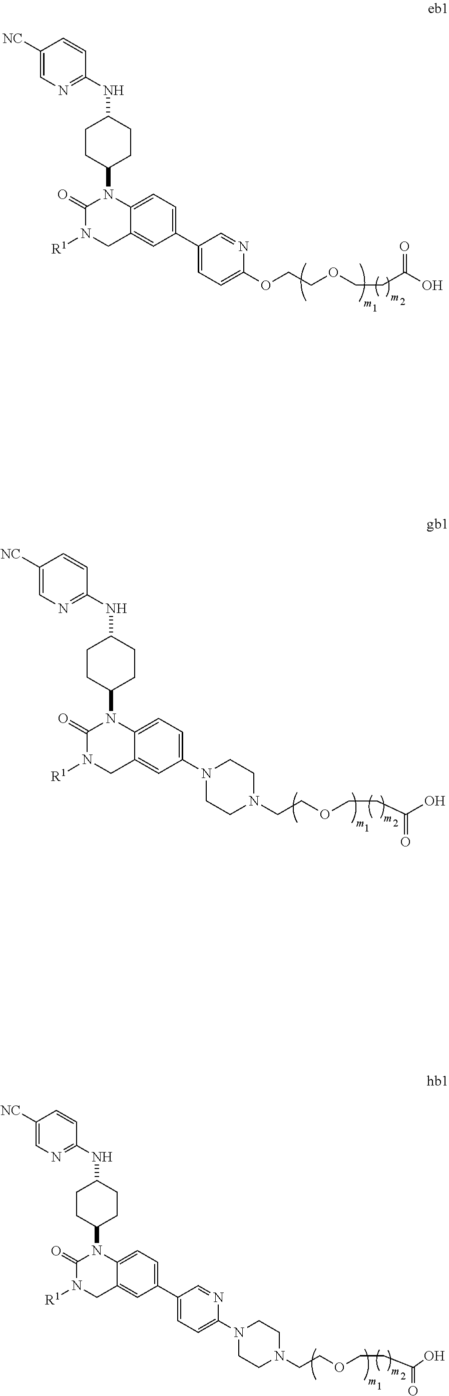

1) Coupling the compound as shown in Formula 1b-1 with bis(pinacolato)diboron to obtain the compound as shown in Formula 2b-1.

Wherein, taking R1 as benzyl as an example, the preparation method for the compound as shown in Formula 1b-1 is as follows: subjecting the compound as shown in Formula 1bb to selective reduction of a cyano group to obtain the compound as shown in Formula 2bb; subjecting the compound as shown in Formula 2bb and the compound as shown in Formula 3bb to a reductive amination reaction to obtain the compound as shown in Formula 4bb; and then reducing the compound as shown in Formula 4bb to obtain the compound as shown in Formula 5bb; the compound as shown in Formula 5bb undergoes intramolecular urea formation to obtain the compound as shown in Formula 6bb; the compound as shown in Formula 6bb and the compound as shown in Formula 7bb undergo a nucleophilic substitution reaction followed by deprotection to obtain the compound as shown in Formula 8bb; and finally, the compound as shown in Formula 8bb and the compound as shown in Formula 6aa undergo a nucleophilic reaction to obtain the compound as shown in Formula 1b-1;

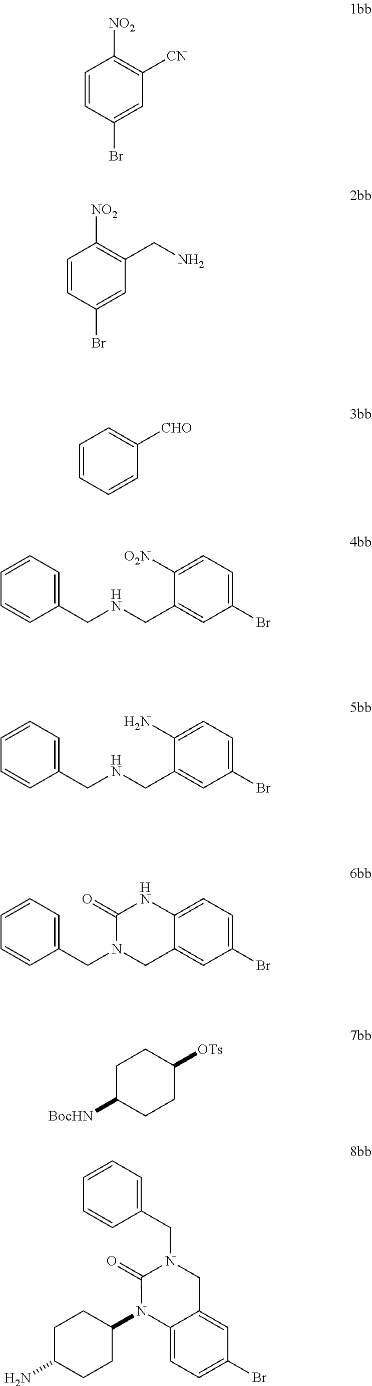

2) The synthesis of the compound as shown in Formula 3cb-1 is obtained by a coupling reaction between the compound as shown in Formula 1b-1 and the compound as shown in Formula 1c, followed by deprotection; the synthesis of the compound as shown in Formula 2db-1 is obtained by a coupling reaction between the compound as shown in Formula 2b-1 and the compound as shown in Formula 1d, followed by removal of the protecting group;

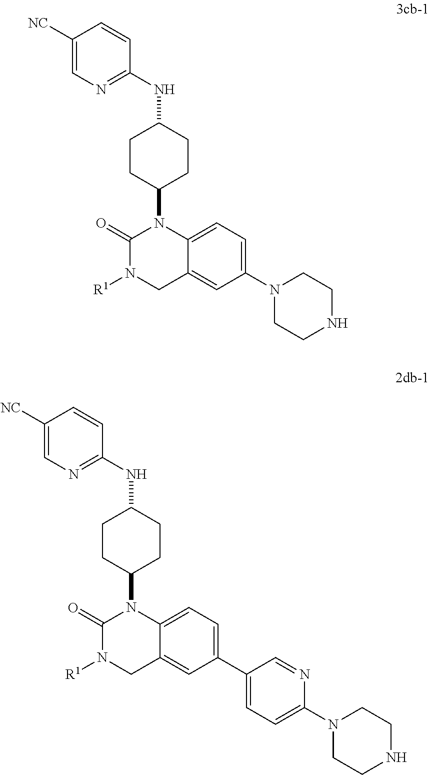

3) The synthesis of the compound as shown in Formula ab1 is by reference to Formula a0, and is obtained by a coupling reaction between the compound as shown in Formula 2b-1 and the compound as shown in Formula 5a, followed by removal of the protecting group;
The synthesis of the compound as shown in Formula cb1 is by reference to Formula c0, and is obtained by a nucleophilic substitution reaction between the compound as shown in Formula 3cb-1 and the compound as shown in Formula 2c, followed by removal of the protecting group;
The synthesis of the compound as shown in Formula db1 is by reference to Formula d0, and is obtained by a nucleophilic substitution reaction between the compound as shown in Formula 2db-1 and the compound as shown in Formula 2c, followed by removal of the protecting group;
The synthesis of the compound as shown in Formula eb1 is by reference to Formula e0, and is obtained by a coupling reaction between the compound as shown in Formula 2b-1 and the compound as shown in Formula e2, followed by removal of the protecting group;
The synthesis of the compound as shown in Formula gb1 is by reference to Formula g0, and is obtained by a nucleophilic substitution reaction between the compound as shown in Formula 3cb-1 and the compound as shown in Formula g1, followed by removal of the protecting group;
The synthesis of the compound as shown in Formula hb1 is by reference to Formula h0, and is obtained by a nucleophilic substitution reaction between the compound as shown in Formula 2db-1 and the compound as shown in Formula g1, followed by removal of the protecting group.

The object of the present invention is to provide a novel and highly efficient CDK12/13 degrader not previously reported in the literature. Unlike conventional inhibitors targeting CDK12/13, the small molecule degrader provided herein efficiently downregulates intracellular CDK12 and CDK13 protein levels, completely inhibits this signaling pathway, significantly inhibits the proliferation of various cancer cells at low concentrations, and has excellent pharmacokinetic properties and reliable drug safety.

Based on the structure of Formula (I), in another aspect, the said T is selected from the structure as shown in Formula (II′):

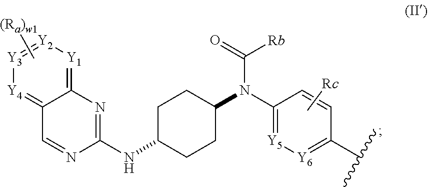

or a pharmaceutically acceptable salt, co-crystal, tautomer, stereoisomer, solvate, hydrate, polymorph, isotopically enriched derivative, or prodrug thereof, wherein:
Y₁, Y₂, Y₃, Y₄, Y₅, and Y₆ are independently selected from CH, C, or N;
Rₐ is selected from C1-C6 alkyl, C1-C6 haloalkyl, C3-C8 cycloalkyl, or C3-C8 heterocyclyl; wherein the alkyl, cycloalkyl, and heterocyclyl can be further substituted with one or more substituents selected from: halogen, C1-C4 alkyl, C1-C4 haloalkyl, C1-C4 alkoxy, cyano, oxo, carboxyl, ester, amido, and hydroxyl;
R_{b} is selected from C1-C6 alkyl, C1-C6 haloalkyl, C3-C8 heterocyclyl, C1-C6 alkoxy, C1-C6 alkoxymethyl, benzyl, cyano, NR_{bb}R_{bc}, or hydroxyl; R_{bb} and R_{bc} are each independently selected from H, C1-C6 alkyl, or C1-C6 haloalkyl; wherein the alkyl, alkoxy, and alkoxymethyl can be further substituted with one or more of the following substituents: hydroxyl, halogen, C1-C4 alkyl, C1-C4 haloalkyl, or an amino group substituted with one or two methyl groups;
R_{c} is selected from H, halogen, C1-C4 alkyl, C1-C4 haloalkyl, C1-C4 alkoxy, cyano, OR_{cc}, oxo, carboxyl, or hydroxyl; R_{cc} is C1-C4 alkyl or C1-C4 haloalkyl;
   [symbol] and [symbol] denote that the two connected N atoms are in a trans configuration relative to the six-membered ring to which they are attached;
   w1 [symbol] 0,1,2,3,4.

Preferably, R_{b} is selected from methyl, ethyl, propyl, C1-C4 fluoroalkyl, C1-C4 hydroxyalkyl, C3-C8 heterocyclyl,

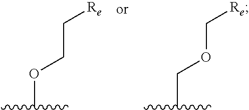

Rₑ is one or more of halogen, C1-C4 alkyl, C1-C4 haloalkyl, or a mono- or di-methyl-substituted amino group.

Further, the specific structure of the said T may be any one of the structures shown below:

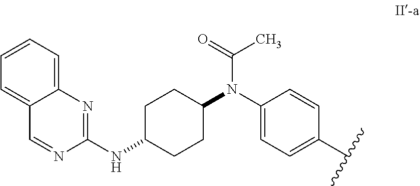 [SMILES: *c1ccc(N(C(C)=O)[C@H]2CC[C@H](Nc3ncc4ccccc4n3)CC2)cc1]

Specifically, E is derived from 3-amino-N-(2,6-dioxo-3-piperidinyl)phthalimide (Pomalidomide), 3-(7-amino-3-oxo-1H-isoindol-2-yl)piperidine-2,6-dione (Lenalidomide), thalidomide, (2S,4R)-1-((S)-2-(3-aminopropanamido)-3,3-dimethylbutanoyl)-4-hydroxy-N-(4-(4-methylthiazol-5-yl)benzyl)pyrrolidine-2-carboxamide (VH032), phenylglutarimide, and the like.

For E, its being derived from a certain group refers to E being a group structure formed by removing a removable H or other leaving groups (such as an amino group) from the corresponding compound of the said group. For example, taking E_{L} as derived from 3-amino-N-(2,6-dioxo-3-piperidinyl)phthalimide, its E refers to the remaining group structure in the compound 3-amino-N-(2,6-dioxo-3-piperidinyl)phthalimide after removing a removable H or other leaving groups (—NH₂).

As a feasible embodiment, E can be selected from one of the following groups:

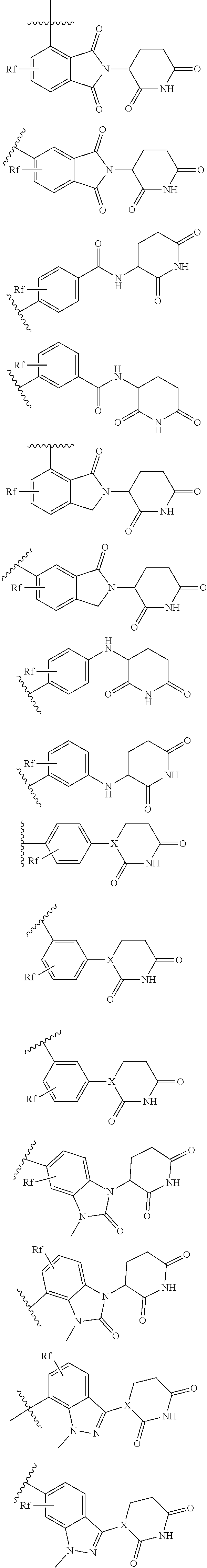

R_{f} is selected from H, F, Cl, CH₃, OMe, CN, CF₃, CHF₂, CH(CH₃)₂, cyclopropyl, and C(O)NH₂;
X is selected from CH and N;
Or, the said E is selected from one of the following structures:

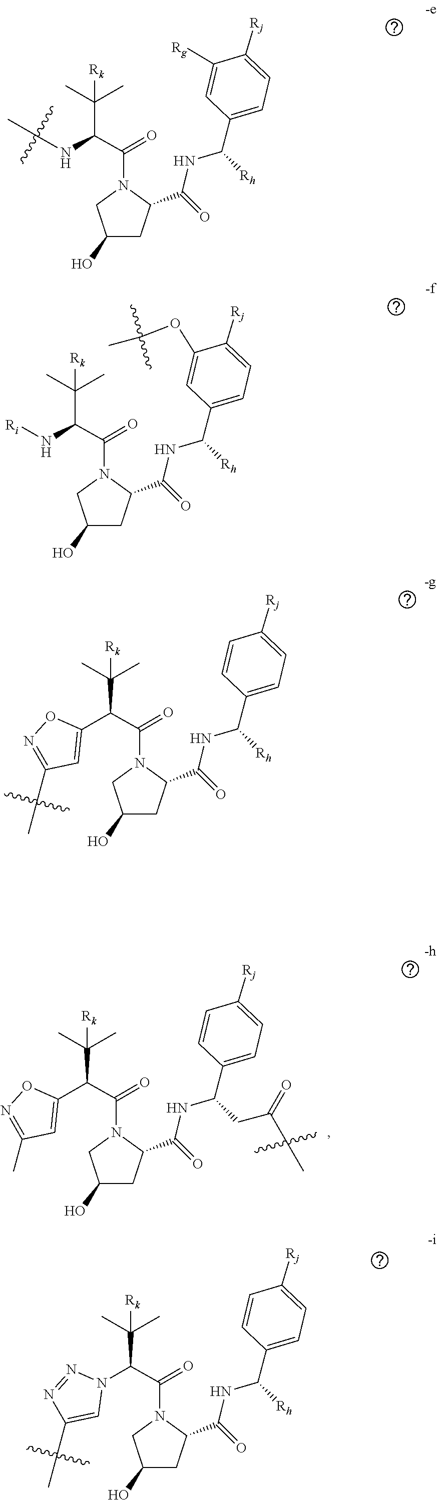

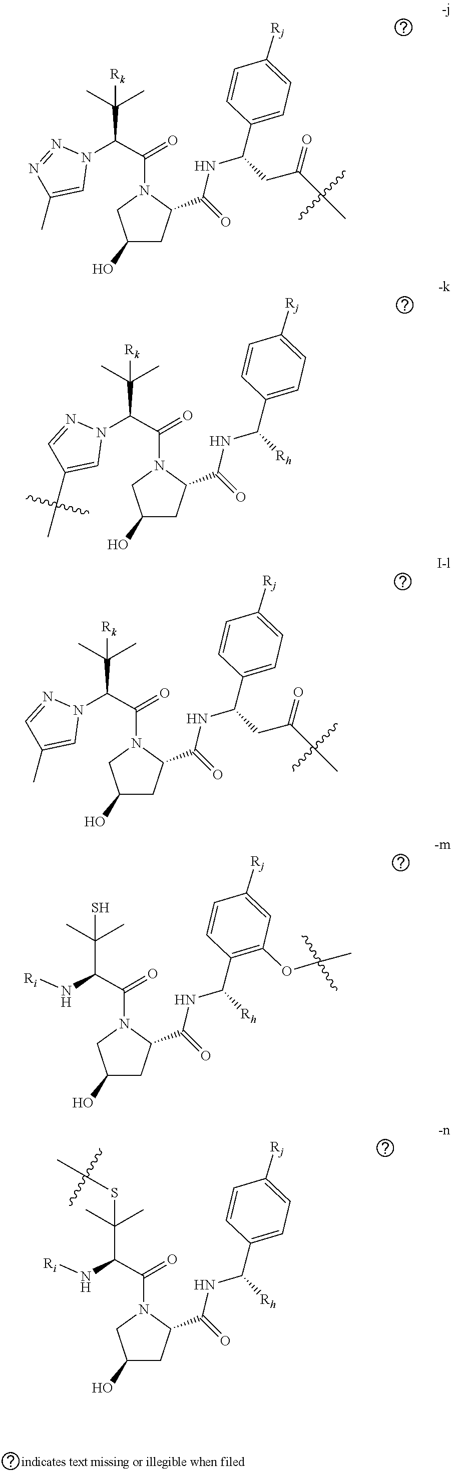

R_{g} is selected from H, OH, Cl, Br, ethynyl, and cyano;
Rₕ is selected from H and CH₃;
Rⁱ is selected from

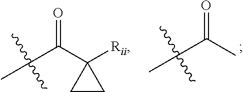

Wherein Rⁱⁱ is —CN, —F, or —COCH₃;
Rⱼ is selected from H, OH, CN, F, Cl, Br, or formula

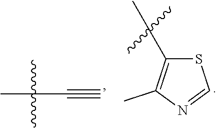

Rₖ is selected from H and CH₃.

Specifically, the said L is a unit of 1 to 30 atoms in length and of any type.

More specifically, the said L includes but is not limited to the following structures, or any structural group composed of the following structures:
A unit composed of an ethylene glycol structure:

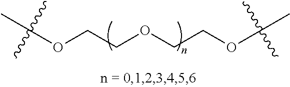

(n is further preferably 0, 1, 2, 3, 4, 5, or 6); Or a unit composed of an aliphatic chain:

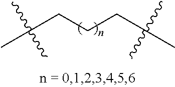

(n is further preferably 0, 1, 2, 3, 4, 5, or 6); Or a unit composed of an unsaturated chain

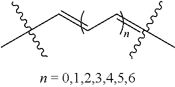

(n is further preferably 0, 1, 2, 3, 4, 5, or 6),

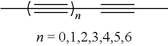

(n is further preferably 0, 1, 2, 3, 4, 5, or 6); Or a unit composed of a non-aromatic heterocyclic ring

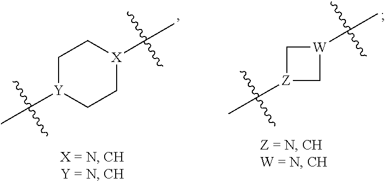

Or a unit composed of an aromatic compound:

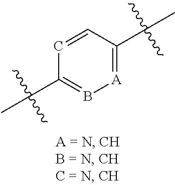

—(CH₂)ₘ— (m is 1, 2, 3, 4), —CHR₂—, —(C═O)—, O,

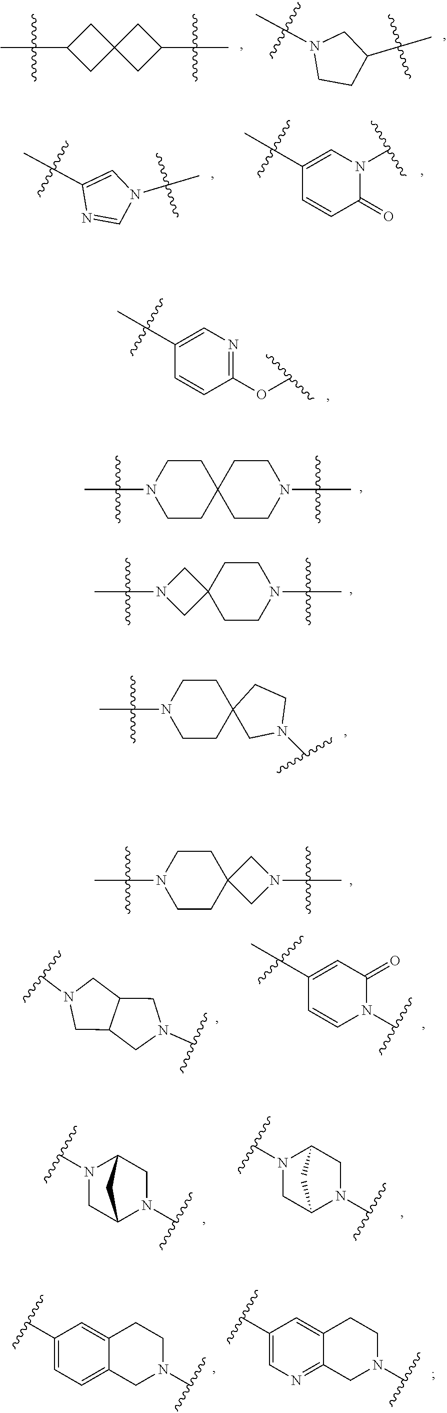

; Or a heteroatom-containing group, wherein the heteroatoms are sulfur, nitrogen, or phosphorus, including but not limited to the following fragment units: —NH—

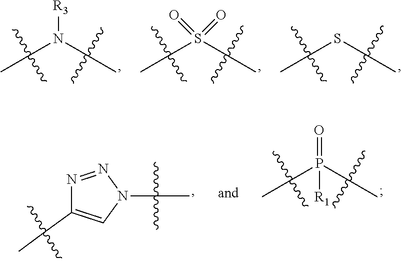

In the said heteroatom-containing unit, R₁ is independently selected from alkyl, alkoxy, or hydrogen;
In the said structural unit, R₂ and R₃ are each independently selected from alkyl and alkoxy;

The said alkyl is specifically methyl, ethyl, propyl, isopropyl, n-butyl, and tert-butyl; the said alkoxy is —OEt or —OMe.

Preferably, the said L is selected from the following structures:

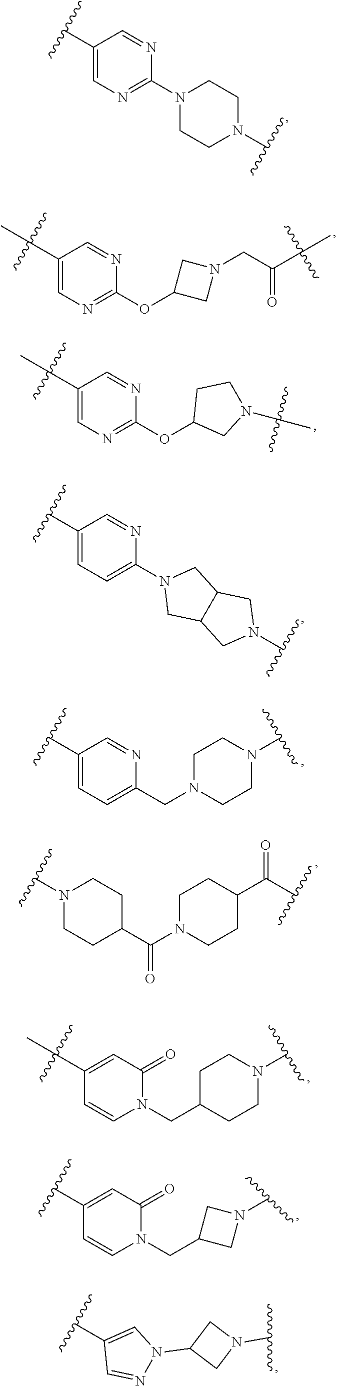

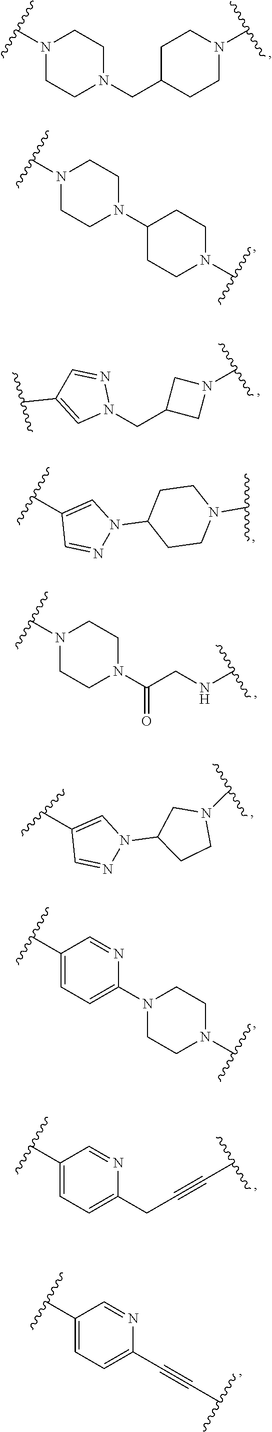

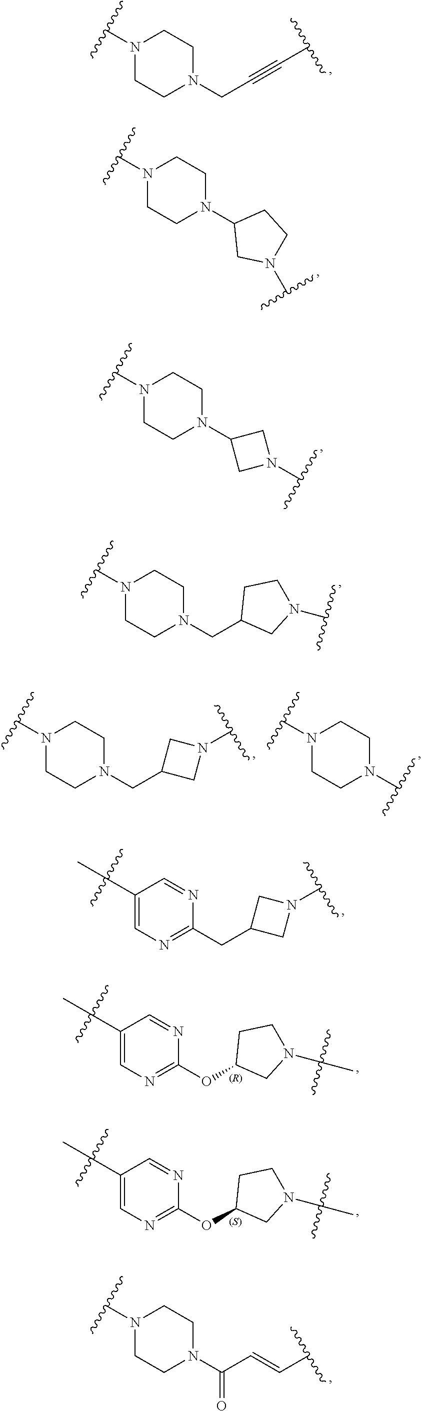

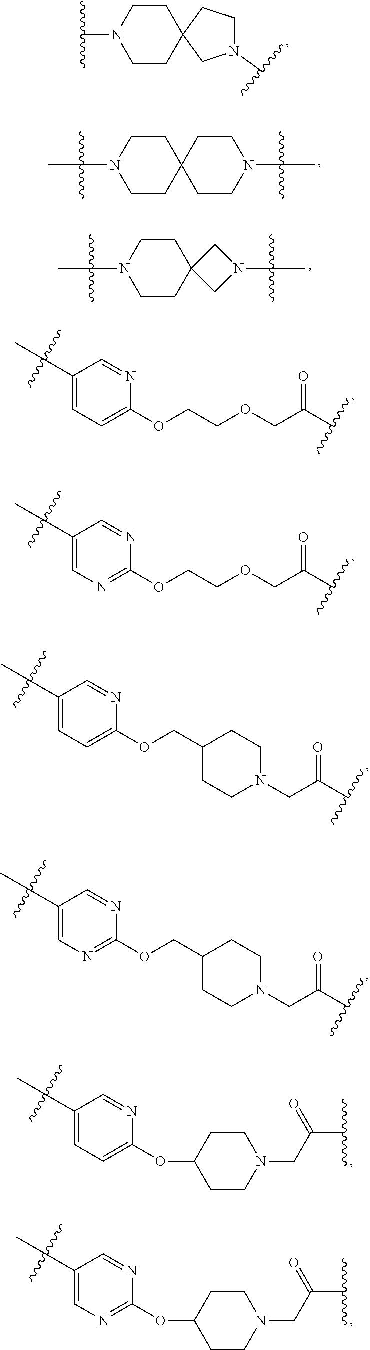

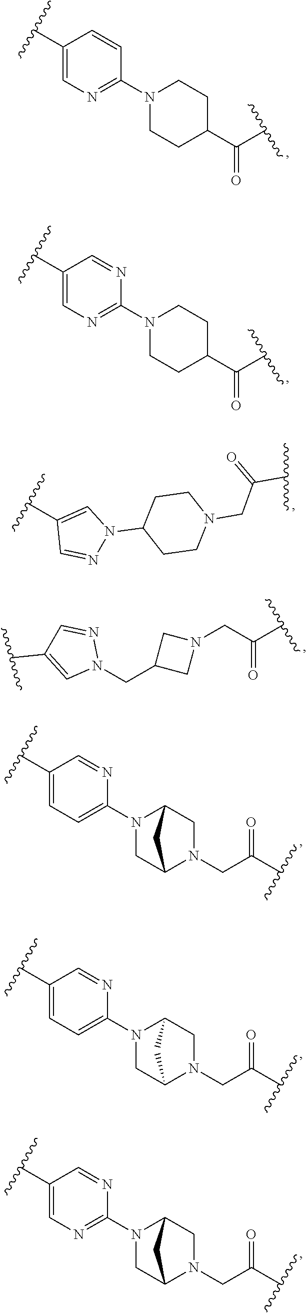

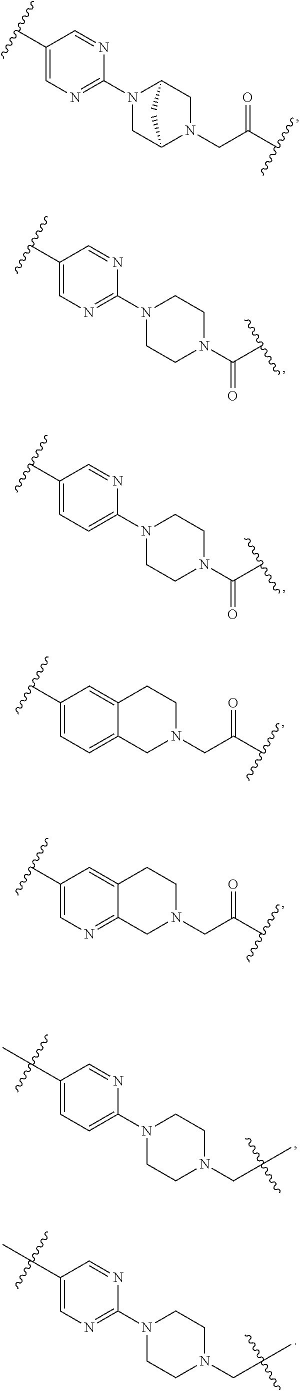

As a preferred embodiment the said L is selected from the following structures:

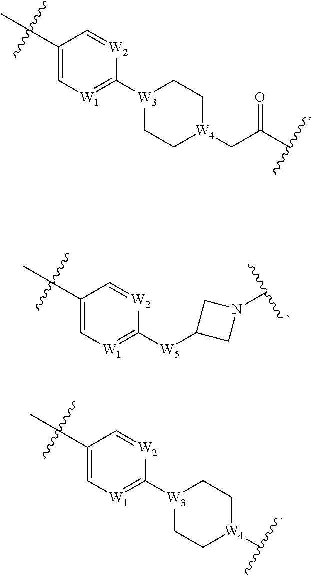

The said degrader drug, cancer cell proliferation inhibitor drug, or CDK12/13 abnormal disease drug can be introduced into the body through methods of injection, spraying, nasal drops, eye drops, osmosis, absorption, or physical or chemical mediation, such as intramuscular, intradermal, subcutaneous, intravenous, or mucosal tissue; or introduced into the body after being mixed or encapsulated with other substances.

As a further preferred embodiment, the said L is selected from the following structures:

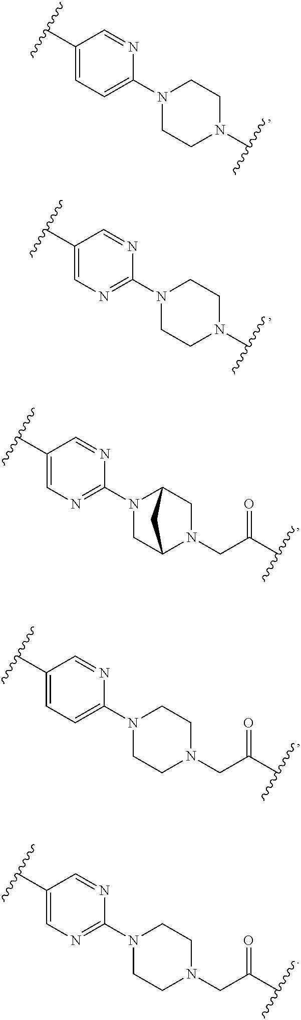 [SMILES: *c1ccc(N2CCN(*)CC2)nc1 ; *c1cnc(N2CCN(*)CC2)nc1 ; *C(=O)CN1C[C@@H]2C[C@H]1CN2c1ncc(*)cn1 ; *C(=O)CN1CCN(c2ccc(*)cn2)CC1 ; *C(=O)CN1CCN(c2ncc(*)cn2)CC1]

Preferably, Rₐ is selected from H, or w is 0.

Preferably, Y₁, Y₂, Y₃, Y₄, Y₅, and Y₆ are all CH.

Preferably, R_{b} is methyl.

Preferably, R_{c}, is H.

Specifically, the said small molecule degrader has a structural formula as shown in any one of the following tables:

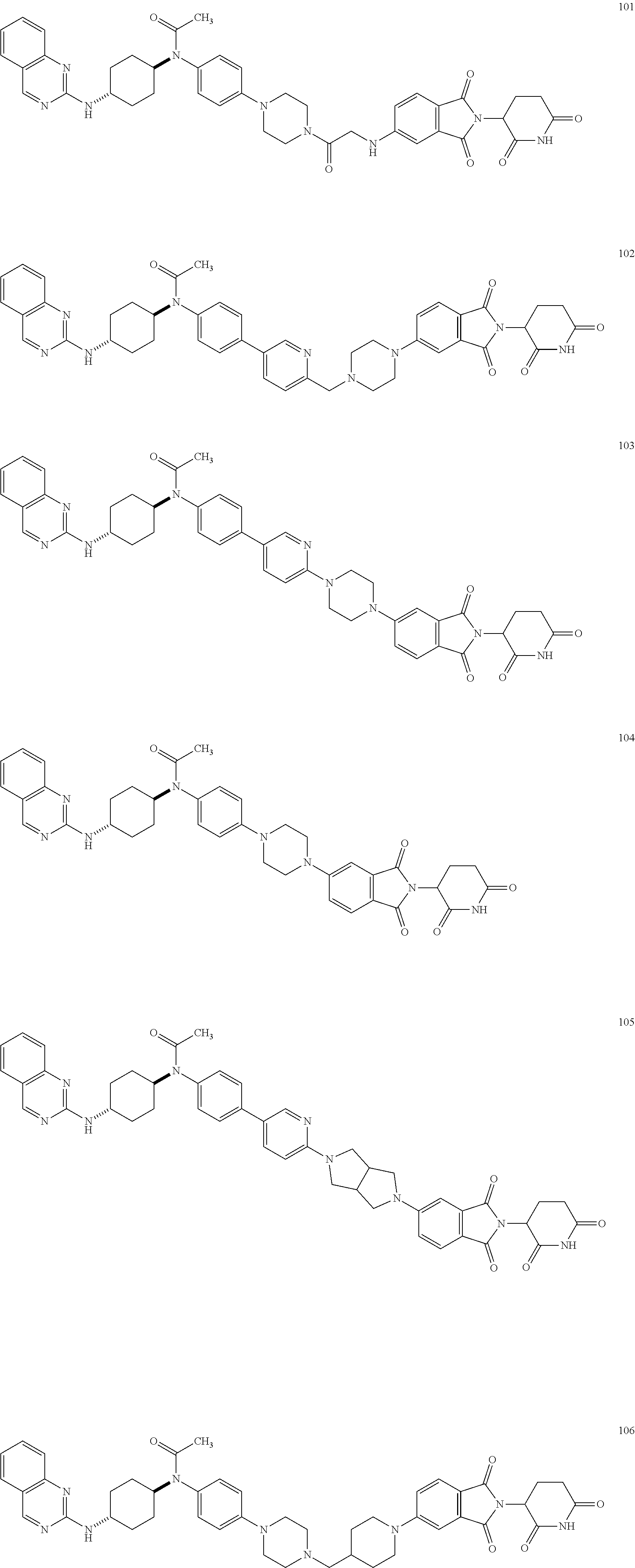

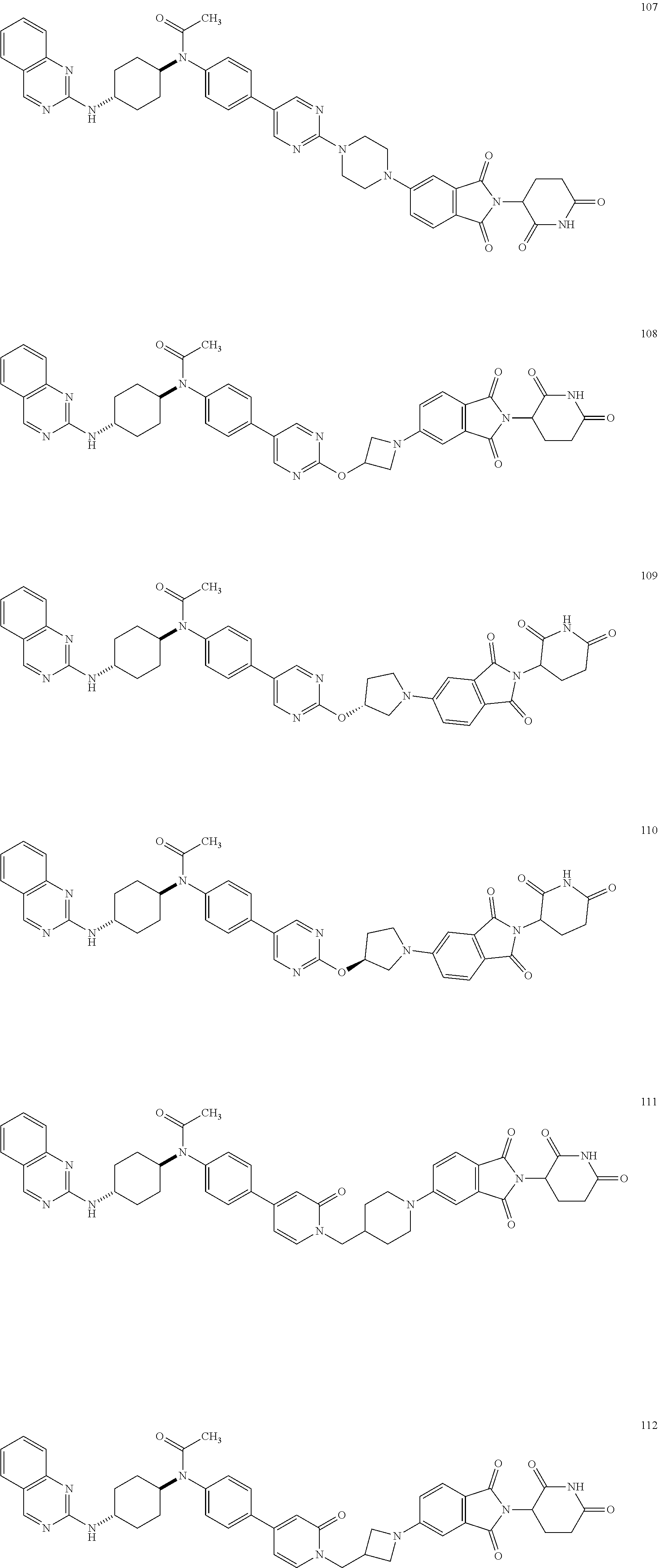

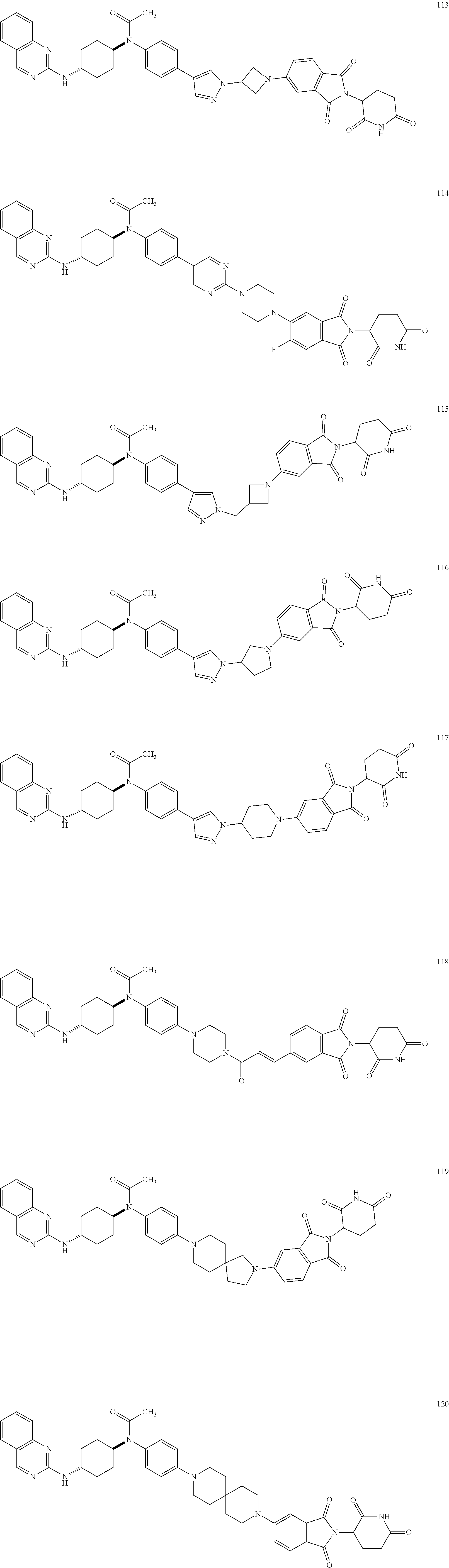

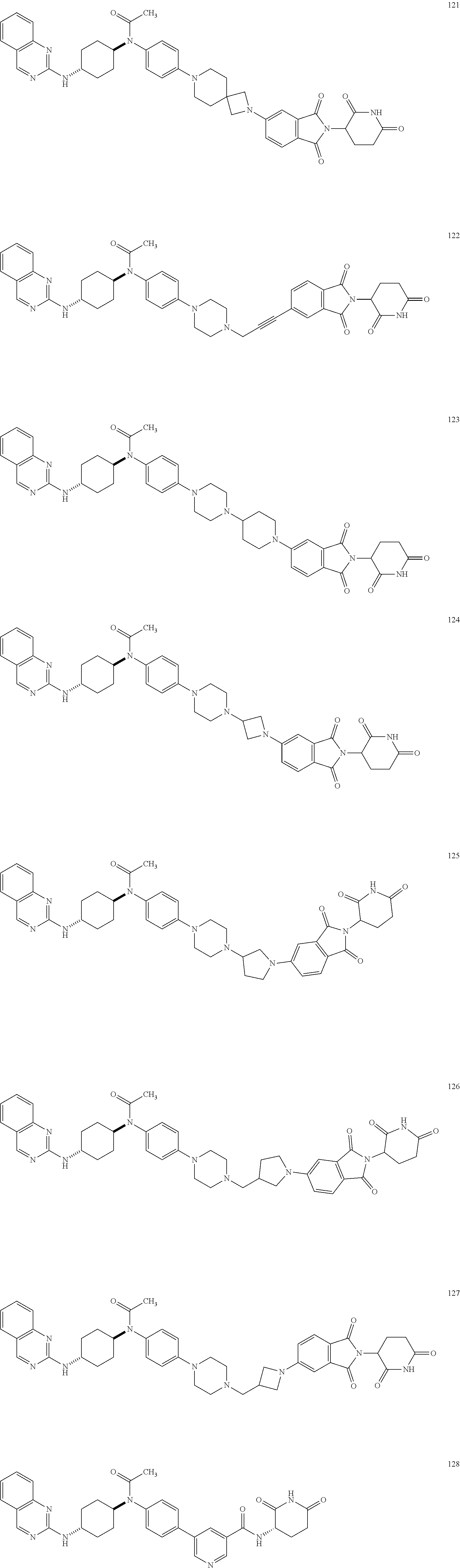

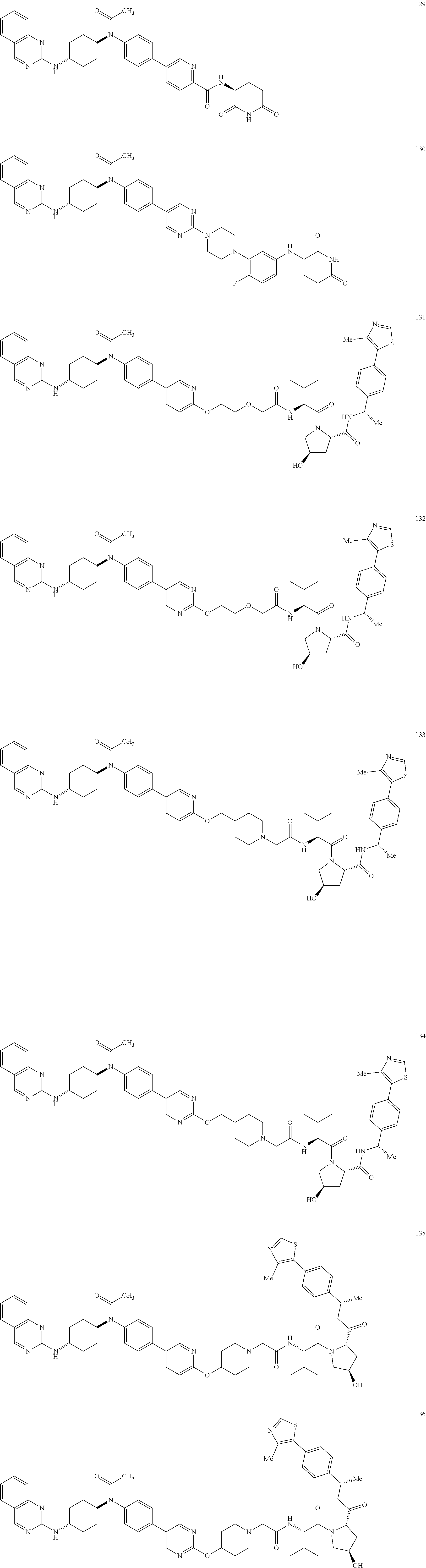

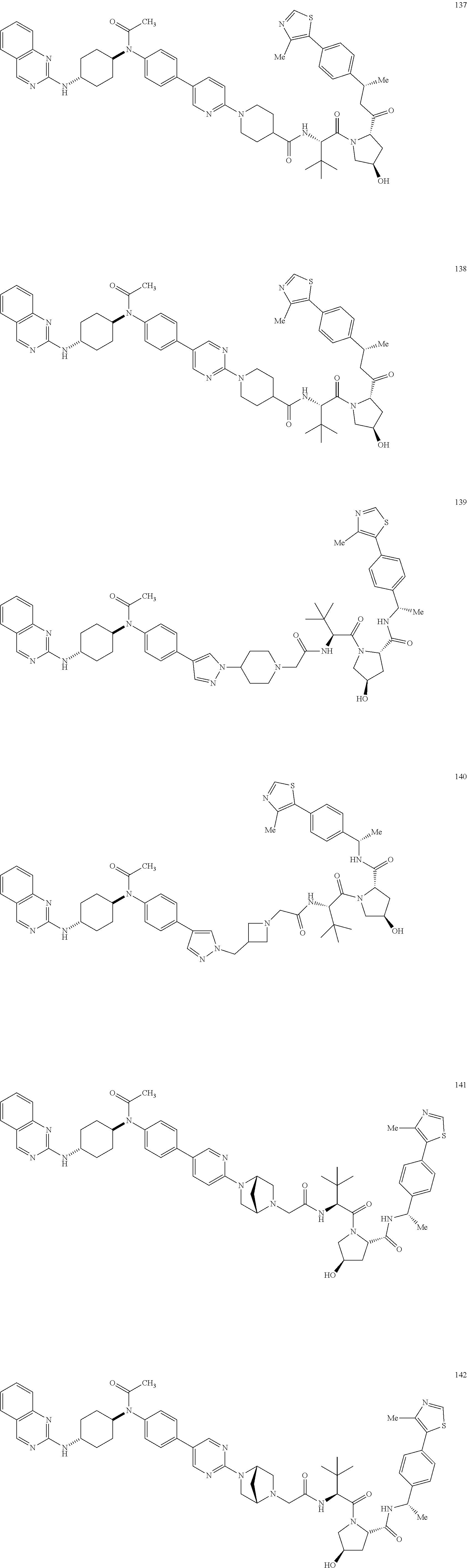

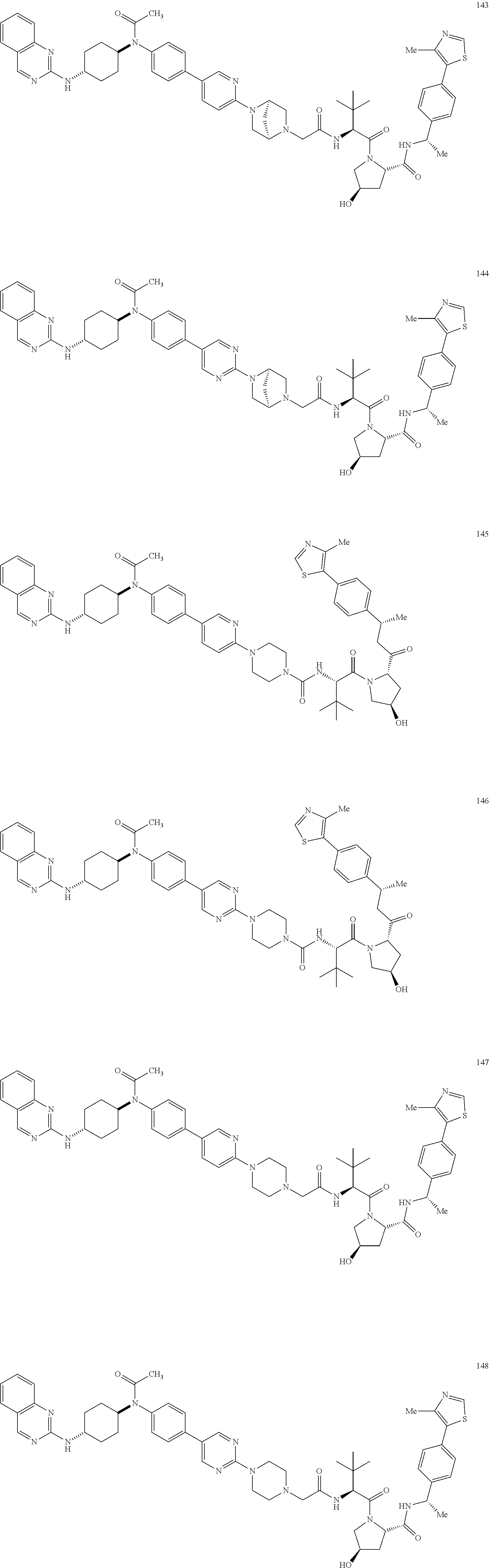

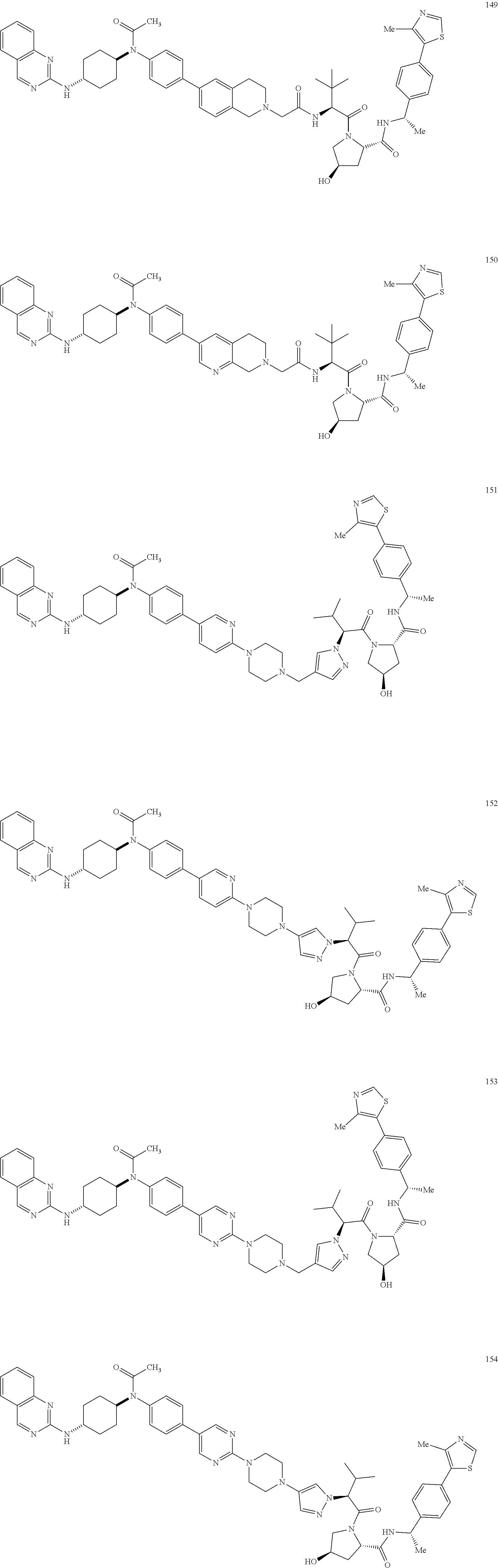

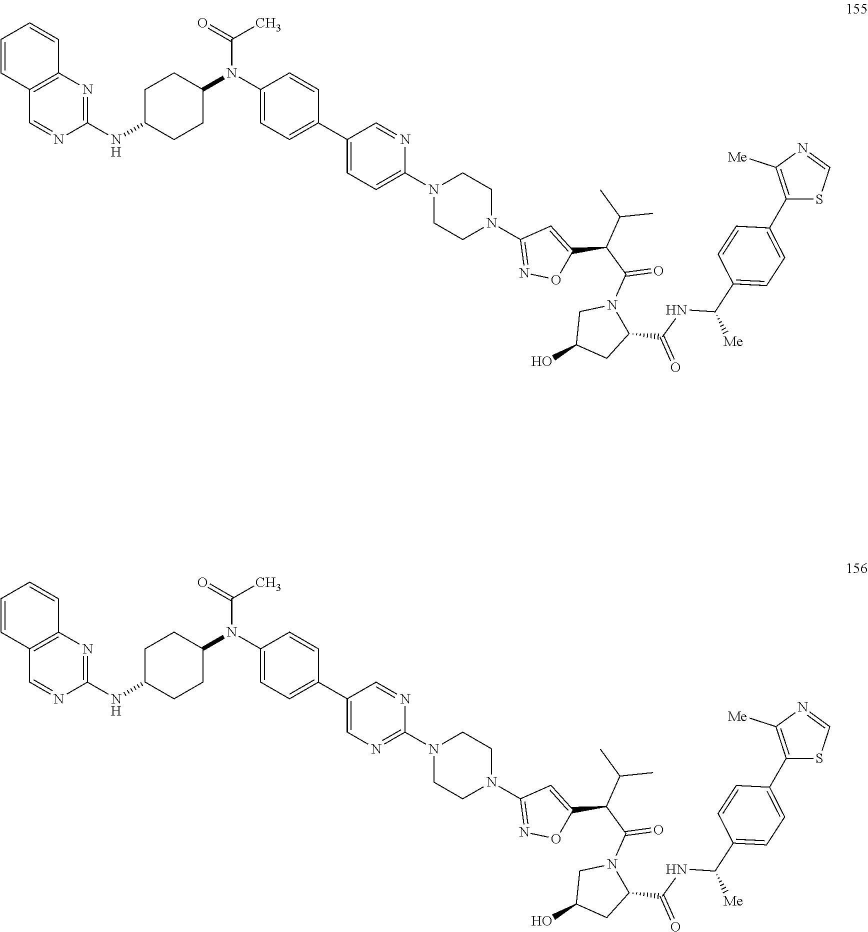

or a stereoisomer thereof, or a stereoisomer mixture thereof, or a pharmaceutically acceptable salt thereof.

Preferably, the said pharmaceutically acceptable salt is selected from one or more of the following

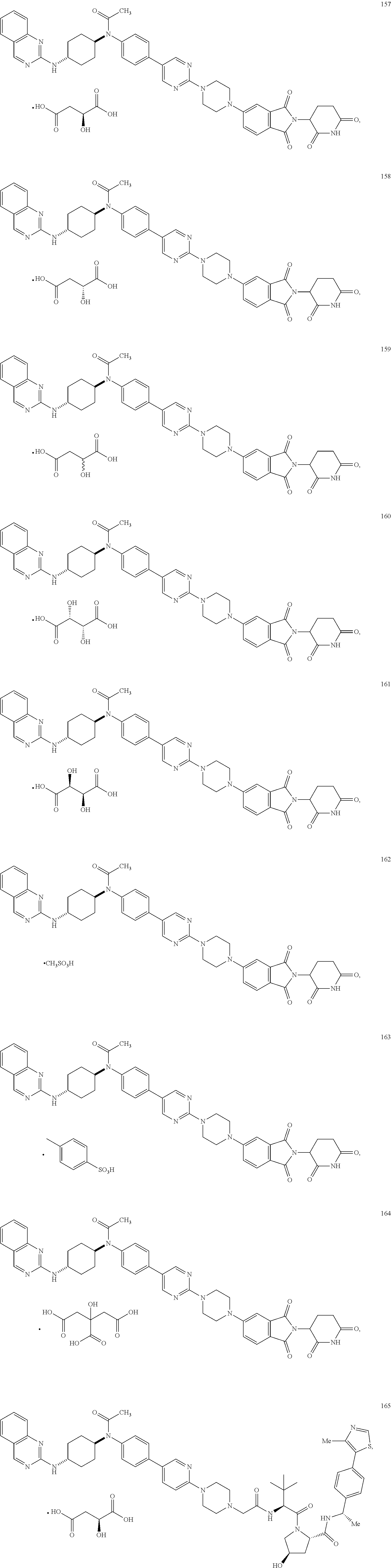

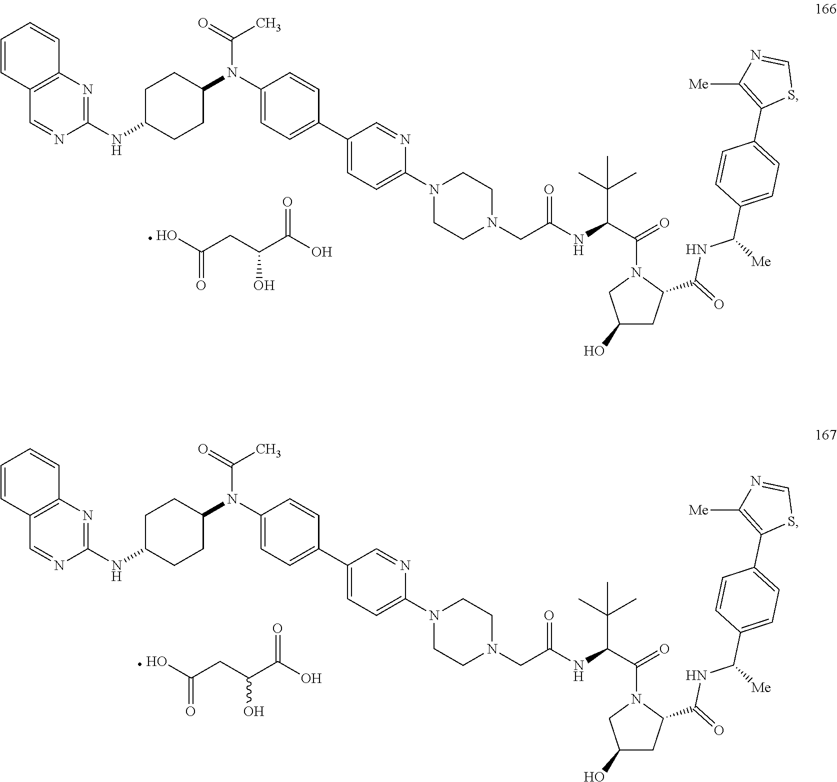 [SMILES: CC(=O)N(c1ccc(-c2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)nc2)cc1)[C@H]1CC[C@H](Nc2ncc3ccccc3n2)CC1 ; O=C(O)C[C@@H](O)C(=O)O ; CC(=O)N(c1ccc(-c2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)nc2)cc1)[C@H]1CC[C@H](Nc2ncc3ccccc3n2)CC1 ; O=C(O)CC(O)C(=O)O]

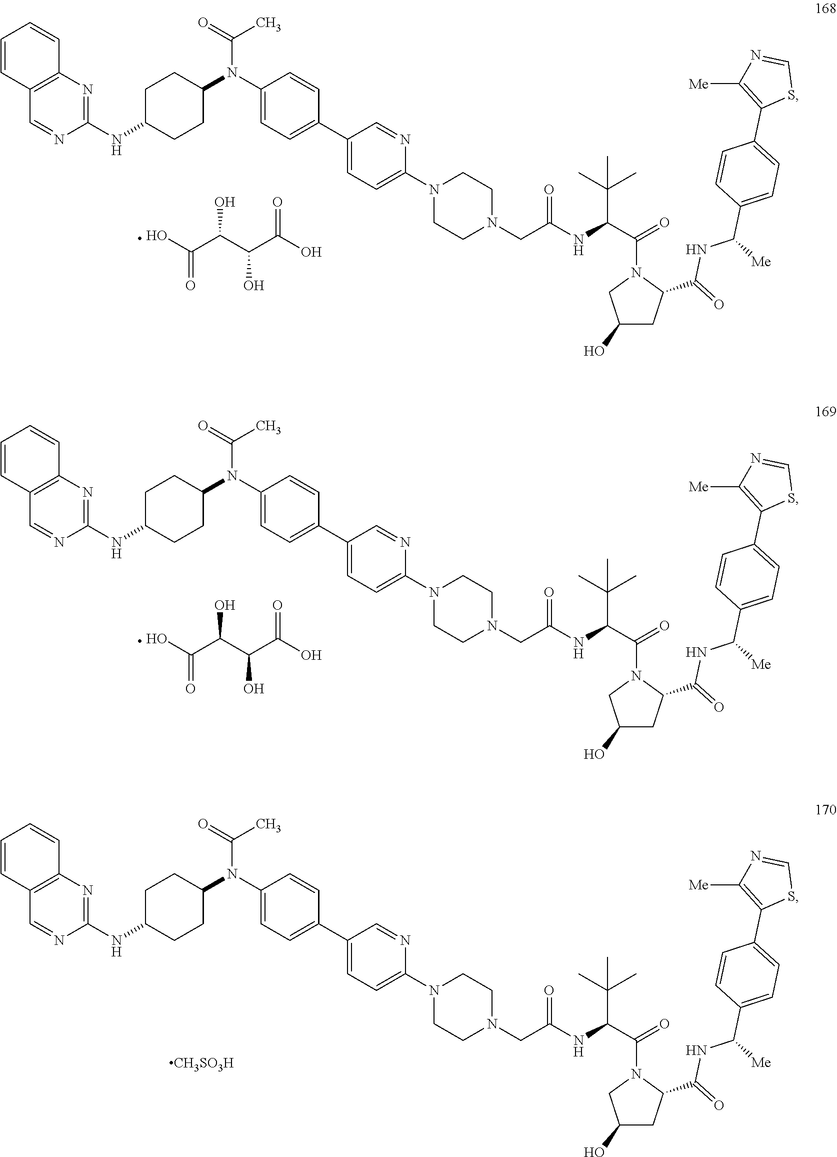

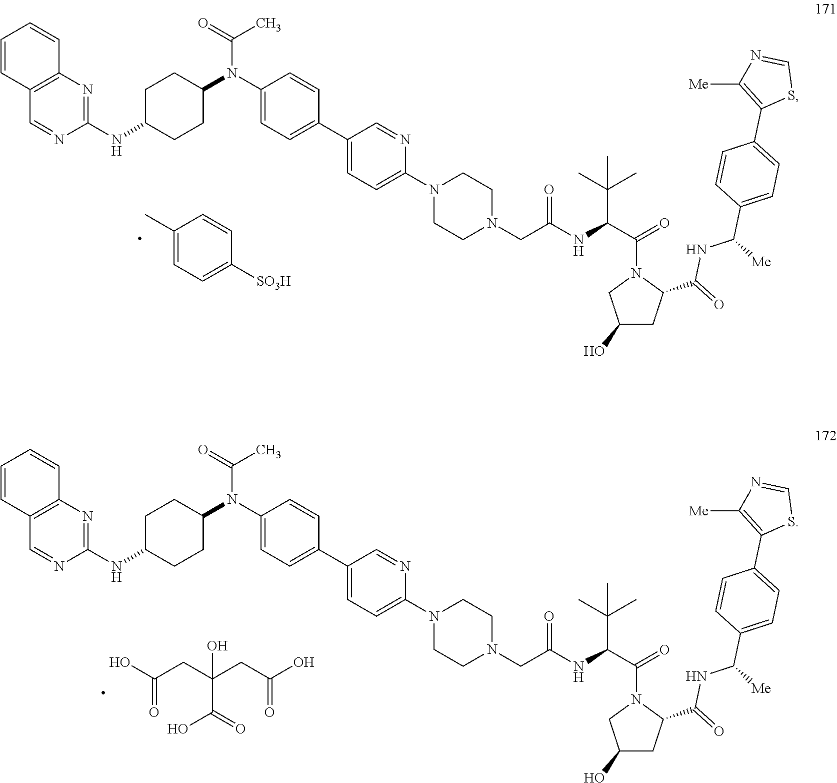 [SMILES: CC(=O)N(c1ccc(-c2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)nc2)cc1)[C@H]1CC[C@H](Nc2ncc3ccccc3n2)CC1 ; Cc1ccc(S(=O)(=O)O)cc1 ; CC(=O)N(c1ccc(-c2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)nc2)cc1)[C@H]1CC[C@H](Nc2ncc3ccccc3n2)CC1 ; O=C(O)CC(O)(CC(=O)O)C(=O)O]

Preferably, the said compounds are the following compounds:
N-(4-(4-((2-(2,6-dioxopiperidin-3-yl)-1,3-dioxoisoindolin-5-yl)aminoacetyl)piperazin-1-yl)phenyl)-N-((1R,4R)-4-(quinazolin-2-ylamino)cyclohexyl)acetamide (101)
N-(4-(6-((4-(2-(2,6-dioxopiperidin-3-yl)-1,3-dioxoisoindolin-5-yl)piperazin-1-yl)methyl)pyridin-3-yl)phenyl)-N-((1R,4R)-4-(quinazolin-2-ylamino)cyclohexyl)acetamide (102)
N-(4-(6-(4-(2-(2,6-dioxopiperidin-3-yl)-1,3-dioxoisoindolin-5-yl)piperazin-1-yl)pyridin-3-yl)phenyl)-N-((1R,4R)-4-(quinazolin-2-ylamino)cyclohexyl)acetamide (103)
N-(4-(4-((1-(2-(2,6-dioxopiperidin-3-yl)-1,3-dioxoisoindolin-5-yl)azetidin-3-yl)methyl)piperazin-1-yl)phenyl)-N-((1R,4R)-4-(quinazolin-2-ylamino)cyclohexyl)acetamide (106)
N-(4-(6-(4-(2-(2,6-dioxopiperidin-3-yl)-1,3-dioxoisoindolin-5-yl)piperazin-1-yl)pyrimidin-3-yl)phenyl)-N-((1R,4R)-4-(quinazolin-2-ylamino)cyclohexyl)acetamide (107)
N-(4-(2-((1-(2-(2,6-dioxopiperidin-3-yl)-1,3-dioxoisoindolin-5-yl)azetidin-3-yl)oxy)pyrimidin-5-yl)phenyl)-N-((1R,4R)-4-(quinazolin-2-ylamino)cyclohexyl)acetamide (108)
N-(4-(2-(((3R)-1-(2-(2,6-dioxopiperidin-3-yl)-1,3-dioxoisoindolin-5-yl)pyrrolidin-3-yl)oxy)pyrimidin-5-yl)phenyl)-N-((1R,4R)-4-(quinazolin-2-ylamino)cyclohexyl)acetamide (109)
N-(4-(1-((1-(2-(2,6-dioxopiperidin-3-yl)-1,3-dioxoisoindolin-5-yl)azetidin-3-yl)methyl)-2-oxo-1,2-dihydropyridin-4-yl)phenyl)-N-((1R,4R)-4-(quinazolin-2-ylamino)cyclohexyl)acetamide (112)
(2S,4R)-1-((S)-3,3-dimethyl-2-(2-((1R,4R)-5-(5-(4-(N-((1R,4S)-4-(quinazolin-2-ylamino)cyclohexyl)acetamido)phenyl)pyridin-2-yl)-2,5-diazabicyclo[2.2.1]heptan-2-yl)acetamido)butanoyl)-4-hydroxy-N—((S)-1-(4-(4-methylthiazol-5-yl)phenyl)ethyl)pyrrolidine-2-carboxamide (143)
(2S,4R)-1-((S)-3,3-dimethyl-2-(2-((1R,4R)-5-(5-(4-(N-((1r,4S)-4-(quinazolin-2-ylamino)cyclohexyl)acetamido)phenyl)pyrimidin-2-yl)-2,5-diazabicyclo[2.2.1]heptan-2-yl)acetamido)butanoyl)-4-hydroxy-N—((S)-1-(4-(4-methylthiazol-5-yl)phenyl)ethyl)pyrrolidine-2-carboxamide (144)
(2S,4R)-1-((S)-3,3-dimethyl-2-(2-(4-(5-(4-(N-((1R,4R)-4-(quinazolin-2-ylamino)cyclohexyl)acetamido)phenyl)pyridin-2-yl)piperazin-1-yl)acetamido)butanoyl)-4-hydroxy-N—((S)-1-(4-(4-methylthiazol-5-yl)phenyl)ethyl)pyrrolidine-2-carboxamide (147)
(2S,4R)-1-((S)-3,3-dimethyl-2-(2-(4-(5-(4-(N-((1R,4R)-4-(quinazolin-2-ylamino)cyclohexyl)acetamido)phenyl)pyrimidin-2-yl)piperazin-1-yl)acetamido)butanoyl)-4-hydroxy-N—((S)-1-(4-(4-methylthiazol-5-yl)phenyl)ethyl)pyrrolidine-2-carboxamide (148)
(2S,4R)-4-hydroxy-1-((S)-3-methyl-2-(4-((4-(5-(4-(N-((1R,4R)-4-(quinazolin-2-ylamino)cyclohexyl)acetamido)phenyl)pyridin-2-yl)piperazin-1-yl)methyl)-1H-pyrazol-1-yl)butanoyl)-N—((S)-1-(4-(4-methylthiazol-5-yl)phenyl)ethyl)pyrrolidine-2-carboxamide (151)
(2S,4R)-4-hydroxy-1-((S)-3-methyl-2-(4-(4-(5-(4-(N-((1R,4R)-4-(quinazolin-2-ylamino)cyclohexyl)acetamido)phenyl)pyridin-2-yl)piperazin-1-yl)-1H-pyrazol-1-yl)butanoyl)-N—((S)-1-(4-(4-methylthiazol-5-yl)phenyl)ethyl)pyrrolidine-2-carboxamide (152)
(2S,4R)-4-hydroxy-1-((S)-3-methyl-2-(4-((4-(5-(4-(N-((1R,4R)-4-(quinazolin-2-ylamino)cyclohexyl)acetamido)phenyl)pyrimidin-2-yl)piperazin-1-yl)methyl)-1H-pyrazol-1-yl)butanoyl)-N—((S)-1-(4-(4-methylthiazol-5-yl)phenyl)ethyl)pyrrolidine-2-carboxamide (153)
(2S,4R)-4-hydroxy-1-((S)-3-methyl-2-(4-(4-(5-(4-(N-((1R,4R)-4-(quinazolin-2-ylamino)cyclohexyl)acetamido)phenyl)pyrimidin-2-yl)piperazin-1-yl)-1H-pyrazol-1-yl)butanoyl)-N—((S)-1-(4-(4-methylthiazol-5-yl)phenyl)ethyl)pyrrolidine-2-carboxamide (154)
(2S,4R)-4-hydroxy-1-((S)-3-methyl-2-(3-(4-(5-(4-(N-((1R,4R)-4-(quinazolin-2-ylamino)cyclohexyl)acetamido)phenyl)pyridin-2-yl)piperazin-1-yl)isoxazol-5-yl)butanoyl)-N—((S)-1-(4-(4-methylthiazol-5-yl)phenyl)ethyl)pyrrolidine-2-carboxamide (155)
(2S,4R)-4-hydroxy-1-((S)-3-methyl-2-(3-(4-(5-(4-(N-((1R,4R)-4-(quinazolin-2-ylamino)cyclohexyl)acetamido)phenyl)pyrimidin-2-yl)piperazin-1-yl)isoxazol-5-yl)butanoyl)-N—((S)-1-(4-(4-methylthiazol-5-yl)phenyl)ethyl)pyrrolidine-2-carboxamide (156).

### Applications

A further object of the present invention is to provide the use of the above small molecule conjugates.

The present invention provides the use of a small molecule conjugate as described in any one of the above technical solutions, or a composition or preparation containing the said small molecule conjugate as mentioned in any one of the following technical solutions, in the preparation of a medicament for single or combined use for the prevention and/or treatment of a disease, disorder, or condition that benefits from the inhibition or degradation of CDK12 and/or CDK13.

Furthermore, a small molecule conjugate as described in any one of the above technical solutions can be used in at least one of the following aspects 1) to 3):
1) its use in the preparation of a CDK12/13 degrader;
2) its use in the preparation of a cancer cell proliferation inhibitor;
3) its use in the preparation of a medicament for the prevention and/or treatment of a CDK12/13 abnormal disease.

The said CDK12/13 degrader can selectively degrade CDK12/13 in, but not limited to, the following cells: colorectal cancer cells (e.g., HCT116, HCT-15), cervical cancer cells (e.g., Hela), ovarian cancer cells (e.g., OVCAR8), breast cancer cells (e.g., BT-549, MDA-MB-231), glioma cells (e.g., U251), acute myeloid leukemia cells (e.g., U937, Kasumi-1, MV-4-11, NB4), T-lymphocyte leukemia cells (e.g., Jurkat, MOLT-4), myeloma cells (e.g., MM.1s), gastric cancer cells, and liver cancer cells.

The said cancer cells include, but are not limited to, the following cells: myeloma cells, breast cancer cells, leukemia cells, glioma cells, colorectal cancer cells, cervical cancer cells, ovarian cancer cells, gastric cancer cells, liver cancer cells, acute myeloid leukemia cells, and T-lymphocyte leukemia cells.

The said CDK12/13 abnormal diseases include, but are not limited to, breast cancer, prostate cancer, gastric cancer, cervical cancer, ovarian cancer, Ewing's sarcoma, leukemia, and leishmaniasis.

The said products include: 1) a CDK12/13 protein degrader; 2) a tumor cell proliferation inhibitor; 3) a medicament for the prevention and/or treatment of a disease, disorder, or condition that benefits from the inhibition or degradation of CDK12 and/or 13.

The said degrader drug, cancer cell proliferation inhibitor drug, or CDK12/13 abnormal disease drug can be introduced into the body through methods of injection, spraying, nasal drops, eye drops, osmosis, absorption, or physical or chemical mediation, such as intramuscular, intradermal, subcutaneous, intravenous, or mucosal tissue; or introduced into the body after being mixed or encapsulated with other substances. When necessary, one or more pharmaceutically acceptable carriers may also be added to the said medicament. That is, the present invention also provides a pharmaceutical composition (combination drug) comprising a compound as described in any one of the above technical solutions and one or more pharmaceutically acceptable carriers. The said carrier includes one or more of the following conventional in the pharmaceutical field: diluents, excipients, fillers, binders, wetting agents, disintegrants, absorption enhancers, surfactants, adsorbent carriers, and lubricants.

The above medicaments can be prepared in various forms, such as tablets, powders, granules, capsules, oral liquids, pastes, creams, and injections; all the above dosage forms of the medicament can be prepared according to conventional methods in the pharmaceutical field.

The present invention also provides a pharmaceutical composition (combination drug) comprising the small molecule conjugate as described in any one of the above technical solutions.

The small molecule conjugate (CDK12/13 degrader) provided by the present invention can be used not only independently for the treatment of tumors but also in combination with other drugs for more effective treatment of diseases.

As one embodiment, when used in combination, the combination drug composition provided by the present invention comprises a small molecule conjugate (CDK12/13, CCNK degrader) as shown in Formula I and at least one of the following drugs: a PARP inhibitor, a BET inhibitor, a CDK4&6 inhibitor, a PI3K inhibitor, and all-trans retinoic acid (ATRA).

The said PARP inhibitors include Olaparib, Rucaparib, Niraparib, and Talazoparib. The BET inhibitors include ABBV-075, JQ1, dBET-1, and ARV-825. The said CDK4&6 inhibitors include YX-2-107 and Palbociclib. The said PI3K inhibitors include Copanlisib.

The said combination drug composition can be a composition comprising the small molecule conjugate (CDK12/13 degrader) as shown in Formula I and all the above inhibitors in a molar ratio of 1:1 to 10:1.

Currently, there is a lack of CDK12/13 inhibitors and degraders with cell type selectivity. The present invention utilizes PROTAC technology to obtain highly active CDK12/13 degraders. These compounds exhibit high-efficiency CDK12/13 degradation activity in various cell lines and have the ability to highly specifically inhibit cancer cell proliferation. The degradation concentration 50% (DC₅₀) for CDK12 and CDK13 is approximately 30 nM and 21 nM, respectively, and the inhibition concentration 50% (IC₅₀) for tumor cells KASUMI-1 and MV4-11 is approximately 170 nM and 90 nM, respectively, but the IC50 in normal cells L-02 and HK-2 is approximately 1000 nM. For some other preferred compounds, the DC₅₀ for CDK12 and CDK13 in triple-negative breast cancer cells MDA-MB-231 is approximately 16 nM and 10 nM, and the IC50 for various tumor cells such as triple-negative breast cancer cells BT549, MDA-MB-231, colorectal cancer cells HCT-116, and ovarian cancer cells OVAR8 is approximately 0.18 μM, 0.16 μM, 0.5 μM, and 0.15 μM, respectively.

The degraders of the present invention demonstrate strong degradation capabilities for CDK12/13 in various cell lines and show highly selective proliferation inhibitory activity against tumor cell lines. In combination therapy, they also exhibit highly effective therapeutic effects on tumor cells. From a biological and therapeutic perspective, they can be used as lead compounds for the treatment of various cancers and other CDK12/13 abnormal diseases.

### DESCRIPTIONS OF DRAWINGS

FIG. 1 is a schematic diagram of the mechanism of PROTACs technology.

FIG. 2 shows the degradation of CDK12 and CDK13 after Jurkat cells were treated for 24 h with 3 μM of IN-2 (parent core molecule), Formulae 46, 47, 48, 49, 60, 61, 59, 32, 33, 34, and 38.

FIG. 3 shows the degradation of CDK12 and CDK13 after Jurkat cells were treated for 24 h with 3 μM of IN-2, Formulae 57, 58, 59, 1, 2, 3, 4, 6, 7, and 8.

FIG. 4 shows the degradation of CDK12 and CDK13 after MV4-11 cells were treated for 24 h with 2 μM of Formulae 40, 39, 18, 13, 11, 12, 48, 45, and 1.

FIG. 5 shows the degradation of CDK12 and CDK13 after KASUMI-1 and MV4-11 cells were treated separately for 24 h with 0.125 μM of Formulae 18, 54, 53, 55, 21, 22, 17, 15, 71, 40, 38, 45, 23, 44, 37, and 30, respectively.

FIG. 6 shows the degradation of CDK12 and CDK13 after MDA-MB-231 cells were treated for 24 h with Formula 18 and Formula 45 at different concentrations.

FIG. 7 shows the change in protein abundance of CDK12 and CDK13 over time after MDA-MB-231 cells were treated with 0.3 μM of Formula 18 and Formula 45.

FIG. 8 shows the degradation of CDK12, CDK13, CDK9, and CDK2 after MDA-MB-231 cells were treated for 24 h with Formula 18 and Formula 45 at different concentrations.

FIG. 9 shows that the degradation of CDK12 and CDK13 by Formula 18 and Formula 45 was reversed after the proteasome inhibitor MG132 was added 12 h before sample collection after 24 h of treatment in MDA-MB-231 cells, indicating that the small molecule conjugates degrade CDK12 and CDK13 through the ubiquitin-proteasome pathway.

FIG. 10 shows the survival of tumor cells MDA-MB-231, MV4-11, and KASUMI-1 and normal cells L-02 and HK-2 after 72 h of treatment with a concentration gradient of THZ-531 (a classic CDK12/13 inhibitor), IN-2, and Formula 18.

FIG. 11 shows the proliferation inhibitory effect on MDA-MB-231 cells when Formula 18 is used in combination with Olaparib at different concentrations.

FIG. 12 shows the stability test results of Formula 18 and Formula 45 in liver microsomes from four different species: human, rat, mouse, and dog.

FIG. 13 shows the degradation of CDK12, CDK13, and CCNK after HCT-116 cells were treated for 24 h with 0.1 μM of Formulae 103, 118, 101, 104, 105, 111, 112, 102, 106, 131, and 135.

FIG. 14 shows the degradation of CDK12, CDK13, and CCNK after MDA-MB-231 cells were treated for 24 h with 0.1 μM of Formulae 117, 119, 120, 123, 113, 115, 116, 121, 124, 108, and 107.

FIG. 15 shows the degradation of CDK12, CDK13, and CCNK after Jurkat cells were treated for 24 h with 0.1 μM of Formulae 139, 140, 133, 141, 143, 147, 148, 152, 151, 153, and 140.

FIG. 16 shows the degradation of CDK12, CDK13, and CCNK after MDA-MB-231 cells were treated for 24 h with Formula 147 and Formula 103 at different concentrations.

FIG. 17 shows the change in protein abundance of CDK12 and CDK13 over time after MDA-MB-231 cells were treated with 0.1 μM of Formula 147 and Formula 103.

FIG. 18 shows that the degradation of CDK12 and CDK13 by Formula 47 and Formula 103 was reversed after the proteasome inhibitor MG132 was added 12 h before sample collection after 24 h of treatment in MDA-MB-231 cells, indicating that the small molecule conjugates degrade CDK12 and CDK13 through the ubiquitin-proteasome pathway.

FIG. 19 shows the effect of a concentration gradient of Formula 147 on the phosphorylation of serine 2 in the C-terminal domain of RNA polymerase II (CTD-pSer2) after 24 h of treatment in MDA-MB-231 cells.

FIG. 20 shows the half-maximal proliferation inhibition concentration (IC50) of Formulae 103, 107, 108, 109, 135, 147, and 152 on leukemia cells KASUMI-1 and MV4-11.

FIG. 21 shows the survival of breast cancer cells BT-549, MDA-MB-231, pancreatic cancer cells PANC1, ovarian cancer cells OVCAR8, and colorectal cancer cells HCT-116, HCT-15 after 72 h of treatment with a concentration gradient of Formula 147.

FIG. 22 shows the proliferation inhibitory effect on MDA-MB-231 cells when Formula 147 is used in combination with Olaparib (a PARP inhibitor) at 0.1 μM.

FIG. 23 shows the concentration-effect relationship for the blocking of potassium ion channels encoded by the human ether-a-go-go-related gene (hERG) as detected by patch-clamp technique after treatment with a concentration gradient of Formula 147.

FIG. 24 shows the change in blood drug concentration over time and related pharmacokinetic data after mice were administered 30 mg/kg of Formula 18 intraperitoneally, with an area under the drug concentration-time curve (AUC) of 62236 μg/mL*h and a half-life of 2.75 hours (n=4).

### SPECIFIC EMBODIMENTS

The present invention is described below through specific embodiments, but the present invention is not limited thereto. Any modifications, equivalent substitutions, and improvements, etc., made within the spirit and principles of the present invention shall be included within the scope of protection of the present invention.

Unless otherwise specified, the materials, reagents, etc. used in the following embodiments are all commercially available.

The quantitative experiments in the following embodiments were all performed in triplicate, and the results were averaged. The IN-2 (parent core molecule) used in the following embodiments has the following structure:

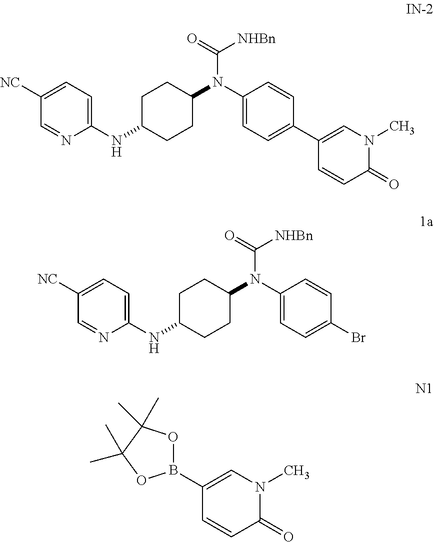

The synthesis route of this compound is: the compound as shown in Formula 1a undergoes a coupling reaction with 1-methyl-5-(4,4,5,5-tetramethyl-1,3,2-dioxaborolan-2-yl)pyridin-2(1H)-one (Formula N1) to obtain it.

The specific synthesis process for the CDK12/13 ligand terminal derivative (as shown in Formula a0) (taking a0-1 as an example (where R¹ is benzyl, n=0, m₁=1, and m₂=0)) is: 1) trans-tert-butyl (4-aminocyclohexyl) carbamate (2aa) undergoes a coupling reaction with 1-bromo-4-iodobenzene (1aa) to obtain tert-butyl ((1R,4R)-4-((4-bromophenyl)amino)cyclohexyl) carbamate (3aa); 2) The compound as shown in Formula ((1R,4R)-4-((4-bromophenyl)amino)cyclohexyl) carbamate undergoes a nucleophilic reaction with benzyl isocyanate (4aa) and the Boc protecting group is removed with hydrochloric acid to obtain 1-((1R,4R)-4-aminocyclohexyl)-3-benzyl-1-(4-bromophenyl)urea (5aa); 3) The 1-((1R,4R)-4-aminocyclohexyl)-3-benzyl-1-(4-bromophenyl)urea undergoes a nucleophilic reaction with 6-fluoronicotinonitrile (6aa) to obtain 3-benzyl-1-(4-bromophenyl)-1-((1R,4R)-4-((5-cyanopyridin-2-yl)amino)cyclohexyl)urea (as shown in Formula 1a); 4) The 3-benzyl-1-(4-bromophenyl)-1-((1R,4R)-4-((5-cyanopyridin-2-yl)amino)cyclohexyl)urea is coupled with bis(pinacolato)diboron to obtain 3-benzyl-1-((1R,4R)-4-((5-cyanopyridin-2-yl)amino)cyclohexyl)-1-(4-(4,4,5,5-tetramethyl-1,3,2-dioxaborolan-2-yl)phenyl)urea (as shown in Formula 2a); 5) 5-bromo-2-fluoropyridine (3a) undergoes a nucleophilic reaction with the compound as shown in Formula 4a to obtain the compound as shown in Formula 5a; 6) The compound as shown in Formula 2a undergoes a coupling reaction with the compound as shown in Formula 5a, and finally the protecting group is removed under acidic conditions to obtain the compound as shown in Formula a0-1.

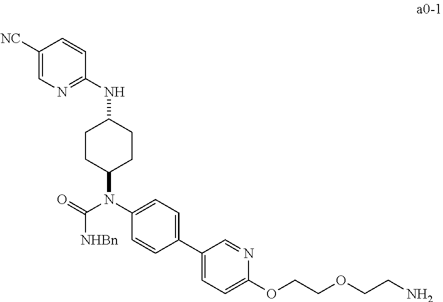

Specifically, when n is 0, the specific preparation process (taking a0-1 as an example (where R¹ is benzyl, n=0, m1=1, and m₂=0)) is as follows:

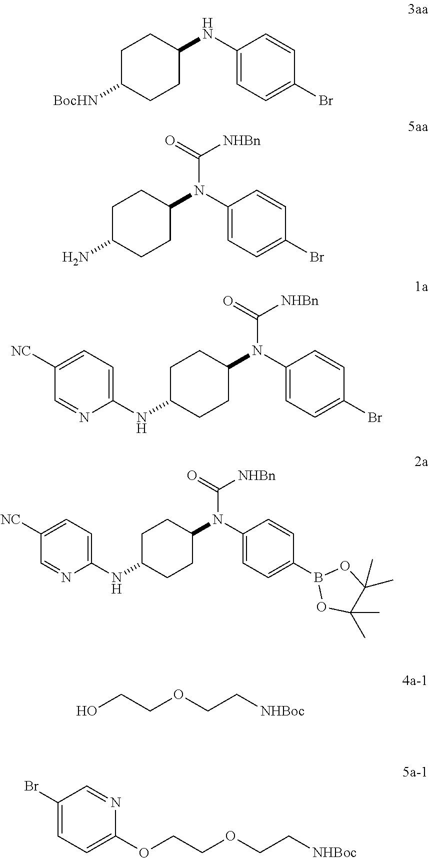

### (1) Preparation of Intermediate 3aa:

In a 250 ml round-bottom flask, 5 g of 1-bromo-4-iodobenzene (1aa), 3.2 g of trans-(4-aminocyclohexyl)carbamic acid tert-butyl ester (2aa), 2.2 g of sodium tert-butoxide, and 856 mg of Xantphos (4,5-bis(diphenylphosphino)-9,9-dimethylxanthene) are added and dissolved in 40 mL of toluene solvent. After purging with nitrogen three times, 542 mg of Pd₂(dba)₃ is added. Purge with nitrogen three more times, heat to 70 °C, and stir for 2 hours. After TLC shows the complete consumption of the starting material, filter with diatomaceous earth, wash the filter cake with ethyl acetate, and concentrate the filtrate under reduced pressure by rotary drying. Purify by silica gel column chromatography to obtain 2.8 g of compound 3aa, with a yield of 67%.

LC-MS (ESI) m/z (M+H)⁺ calculated for C₁₇H₂₆BrN₂O₂⁺=369.1; found 369.2.

¹H NMR (400 MHz, DMSO-d6) δ 7.15 (d, J=8.8 Hz, 2H), 6.79 (d, J=7.9 Hz, 1H), 6.49 (d, J=8.9 Hz, 2H), 5.62 (d, J=8.0 Hz, 1H), 3.25-3.15 (m, 1H), 3.13-2.99 (m, 1H), 1.93 (d, J=11.5 Hz, 2H), 1.78 (d, J=11.0 Hz, 2H), 1.37 (s, 9H), 1.30-1.19 (m, 2H), 1.18-1.07 (m, 2H).

### (2) Preparation of Intermediate 5aa:

In a 125 mL round-bottom flask, 2.8 g of intermediate 3aa is dissolved in 30 mL of anhydrous tetrahydrofuran, and 2.4 g of anhydrous triethylamine is added. After purging with nitrogen three times, stir at room temperature for 5 minutes, then add 4.0 g of benzyl isocyanate (4aa) dropwise to the reaction mixture. Heat to 60 °C and stir for 8 hours. After TLC shows the complete consumption of the starting material, filter with diatomaceous earth, wash the filter cake with ethyl acetate, and concentrate the filtrate under reduced pressure by rotary drying.

After purification by silica gel column chromatography, treat with a 2N hydrogen chloride-ethyl acetate solution. After TLC shows the complete consumption of the starting material, distill the reaction solvent under reduced pressure to obtain 3.4 g of compound 5aa, with a yield of 90%.

LC-MS (ESI) m/z (M+H)⁺ calculated for C₂₀H₂₅BrN₃O⁺=402.1; found 402.1.

¹H NMR (400 MHz, DMSO-d6) δ 7.61 (d, J=8.5 Hz, 2H), 7.25 (t, J=7.4 Hz, 2H), 7.15 (t, J=8.9 Hz, 3H), 7.09 (d, J=8.5 Hz, 2H), 6.67 (d, J=7.9 Hz, 1H), 5.99 (t, J=5.9 Hz, 1H), 4.19-4.09 (m, 3H), 2.99-2.87 (m, 1H), 1.71 (d, J=9.6 Hz, 4H), 1.32 (s, 9H), 1.25-1.17 (m, 2H), 1.03-0.94 (m, 2H).

### (3) Preparation of Intermediate 1a:

In a 100 mL round-bottom flask, 3.4 g of intermediate 5aa and 1.8 g of 6-fluoronicotinonitrile (6aa) are dissolved in anhydrous N-methylpyrrolidone solution. Cool the reaction system to 0 °C, then add 5.3 g of DIPEA (N,N-diisopropylethylamine) dropwise to the reaction mixture. Afterward, warm to 100 °C and stir for 8 hours.

After TLC shows the complete consumption of the starting material, pour the reaction mixture into 100 ml of water and extract three times with 60 mL of dichloromethane. Concentrate the organic phase under reduced pressure. Suspend the resulting solid in ethyl acetate and stir at room temperature for 15 minutes. Filter the suspension through a suction funnel, and wash the filter cake with 50% ethyl acetate/petroleum ether. The obtained solid filter cake is vacuum dried to yield 2.2 g of product 1a, with a yield of 64%.

LC-MS (ESI) m/z (M+H)⁺ calculated for C₂₆H₂₇BrN₅O₁⁺=504.1; found 504.1.

¹H NMR (400 MHz, Chloroform-d) δ 8.27 (s, 1H), 7.56 (d, J=8.5 Hz, 2H), 7.40 (dd, J=8.8, 2.1 Hz, 1H), 7.29 (t, J=7.2 Hz, 2H), 7.23 (d, J=7.1 Hz, 1H), 7.16 (d, J=7.1 Hz, 2H), 7.06 (d, J=8.5 Hz, 2H), 6.26 (d, J=8.8 Hz, 1H), 5.19 (d, J=8.0 Hz, 1H), 4.64-4.49 (m, 1H), 4.36 (d, J=5.8 Hz, 2H), 4.27 (t, J=5.8 Hz, 1H), 3.53 (s, 1H), 2.10 (d, J=11.8 Hz, 2H), 1.95 (d, J=11.5 Hz, 2H), 1.41 (q, J=13.4 Hz, 2H), 1.25 (q, J=15.1, 13.8 Hz, 2H).

### (4) Preparation of Intermediate 2a:

In a 100 mL round-bottom flask, 1.0 g of intermediate 1a, 1.3 g of bis(pinacolato)diboron, and 779 mg of potassium acetate are added and dissolved in anhydrous dioxane solution. After purging with nitrogen three times, 325 mg of Pd(dppf)₂Cl₂ is added. Purge with nitrogen three more times, heat to 80 °C, and stir for 1.5 hours.

After TLC shows the complete consumption of the starting material, wash twice with saturated saline and separate the organic phase. Concentrate the organic phase under reduced pressure and rotary dry. Purify by silica gel column chromatography to obtain 1.0 g of compound 2a, with a yield of 91%.

LC-MS (ESI) m/z (M+H)⁺ calculated for C₃₂H₃₉BrN₅O₃⁺=552.3; found 552.3.

¹H NMR (400 MHz, DMSO-d6) δ 8.28 (d, J=2.4 Hz, 1H), 7.75 (d, J=8.1 Hz, 2H), 7.60 (dd, J=8.9, 2.3 Hz, 1H), 7.48 (d, J=7.7 Hz, 1H), 7.27 (t, J=7.5 Hz, 2H), 7.23-7.12 (m, 4H), 6.46 (d, J=8.9 Hz, 1H), 5.83 (t, J=6.1 Hz, 1H), 5.76 (s, 1H), 4.34-4.21 (m, 1H), 4.13 (d, J=6.0 Hz, 2H), 3.45 (m, J=26.2 Hz, 1H), 1.90 (d, J=12.1 Hz, 2H), 1.83-1.75 (d, 2H), 1.30 (s, 12H), 1.17-1.07 (m, J=35.8 Hz, 4H).

(5) Preparation of Intermediate 4a (Taking 4a-1 as an Example (where m₁=1 and m₂=0)):

In a 100 mL round-bottom flask, 2.0 g of 2-(2-aminoethoxy)ethanol and 2.8 g of triethylamine are added and dissolved in dichloromethane. Cool the reaction system to 0 °C, then add Boc anhydride dropwise to the reaction mixture. Afterward, slowly warm to room temperature and stir for 3 hours. After TLC shows the complete consumption of the starting material, wash twice with a saturated saline bath, and separate the organic phase. Concentrate the organic phase under reduced pressure and vacuum dry. The resulting reaction solution can be used directly for the next step.

LC-MS (ESI) m/z (M+H)⁺ calculated for C₉H₂₀NO₄⁺=205.1; found205.2.

¹H NMR (400 MHz, Chloroform-d) δ 5.29 (s, 1H), 4.22 (t, 2H), 3.71 (t, 2H), 3.48 (t, 2H), 3.21-3.26 (m, 2H), 2.26 (s, 1H), 1.41 (s, 9H).

The preparation of other intermediates (4a-0, 4a-2, 4a-3, 4a-4, and 4a-5) is by reference to 4a-1:
4a-0 (m₁=0, m₂=0): starting material is N-Boc-ethanolamine;
4a-2 (m₁=2, m₂=0): starting material is 2-[2-(2-aminoethoxy)ethoxy]ethanol;
4a-3 (m₁=3, m₂=0): starting material is 2-(2-(2-(2-aminoethoxy)ethoxy)ethoxy)ethanol;
4a-4 (m₁=4, m₂=0): starting material is 14-amino-3,6,9,12-tetraoxatetradecan-1-ol;
4a-5 (m₁=5, m₂=0): starting material is 17-amino-3,6,9,12,15-pentaoxaheptadecanol.
   (1) Preparation of Intermediate 5a-1 (Taking 5a-1 as an Example (where m₁=1 and m₂=0)):

In a 100 mL round-bottom flask, 1 g of 2-(2-Boc-aminoethoxy)ethanol (4a-1), 1.1 g of 2-fluoro-5-bromopyridine, and 4.8 g of cesium carbonate are added and dissolved in anhydrous dimethyl sulfoxide. Heat the reaction system to 100 °C and stir for 6 hours.

After TLC shows the complete consumption of the starting material, pour the reaction mixture into 100 ml of saturated saline and extract three times with 60 ml of ethyl acetate. Concentrate the organic phase under reduced pressure and rotary dry. Purify by silica gel column chromatography to obtain 1.42 g of compound 5a-1, with a yield of 82%.

LC-MS (ESI) m/z (M+H)⁺ calculated for C₁₄H₂₂BrN₂O₄⁺=361.1; found 361.1.

¹H NMR (400 MHz, Chloroform-d) δ 7.48 (d, J=2.7 Hz, 1H), 7.38 (dd, J=9.7, 2.8 Hz, 1H), 6.50 (d, J=10.2 Hz, 1H), 4.99 (s, 1H), 4.28 (t, 2H), 3.75 (t, 2H), 3.50 (t, 2H), 3.22-3.25 (m, 2H), 1.40 (s, 9H).

The preparation of other intermediates (5a-0, 5a-2, 5a-3, 5a-4, and 5a-5) is by reference to 5a-1:
5a-0 (m₁=0, m₂=0): the starting material is replaced with 4a-0 instead of 4a-1;
5a-2 (m₁=2, m₂=0): the starting material is replaced with 4a-2 instead of 4a-1;
5a-3 (m₁=3, m₂=0): the starting material is replaced with 4a-3 instead of 4a-1;
5a-4 (m₁=4, m₂=0): the starting material is replaced with 4a-4 instead of 4a-1;
5a-5 (m₁=5, m₂=0): the starting material is replaced with 4a-5 instead of 4a-1.
   (2) Preparation of the Compound as Shown in Formula a0-1:

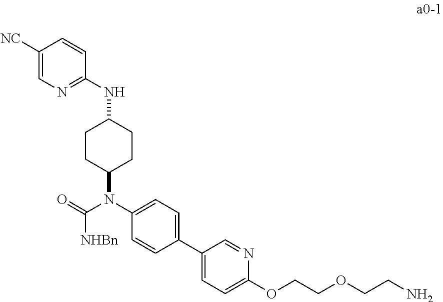

In a 25 mL round-bottom flask, 80 mg of intermediate 2a, 63 mg of intermediate 5a-1, and 142 mg of cesium carbonate are added and dissolved in a mixed solvent of toluene:ethanol:water=4:4:1. After purging with nitrogen three times, 16 mg of Pd(PPh₃)₄ is added. Purge with nitrogen three more times, heat to 100 °C, and stir for 3 hours.

After TLC shows the complete consumption of the starting material, extract three times with 20 mL of dichloromethane. Concentrate the organic phase under reduced pressure and rotary dry. Purify by silica gel column chromatography to obtain 46 mg. Treat with a 2N hydrogen chloride-ethyl acetate solution to obtain compound a0-1, with a yield of 47%.

LC-MS (ESI) m/z (M+H)⁺ calculated for C₃₅H₄₀N₇O₃⁺=606.3; found 606.4.

The preparation of other intermediates (a-0, a-2, a-3, a-4, and a-5) is by reference to a-1.
a0-0 (m₁=0, m₂=0): the starting material is replaced with 5a-0 instead of 5a-1;
a0-2 (m₁=2, m₂=0): the starting material is replaced with 5a-2 instead of 5a-1;
a0-3 (m₁=3, m₂=0): the starting material is replaced with 5a-3 instead of 5a-1;
a0-4 (m₁=4, m₂=0): the starting material is replaced with 5a-4 instead of 5a-1;
a0-5 (m₁=5, m₂=0): the starting material is replaced with 5a-5 instead of 5a-1;
When n is not 0, the specific preparation process (taking ab1-1 as an example (where R¹ is benzyl, n=1, m₁=1, and m₂=0)) is as follows:

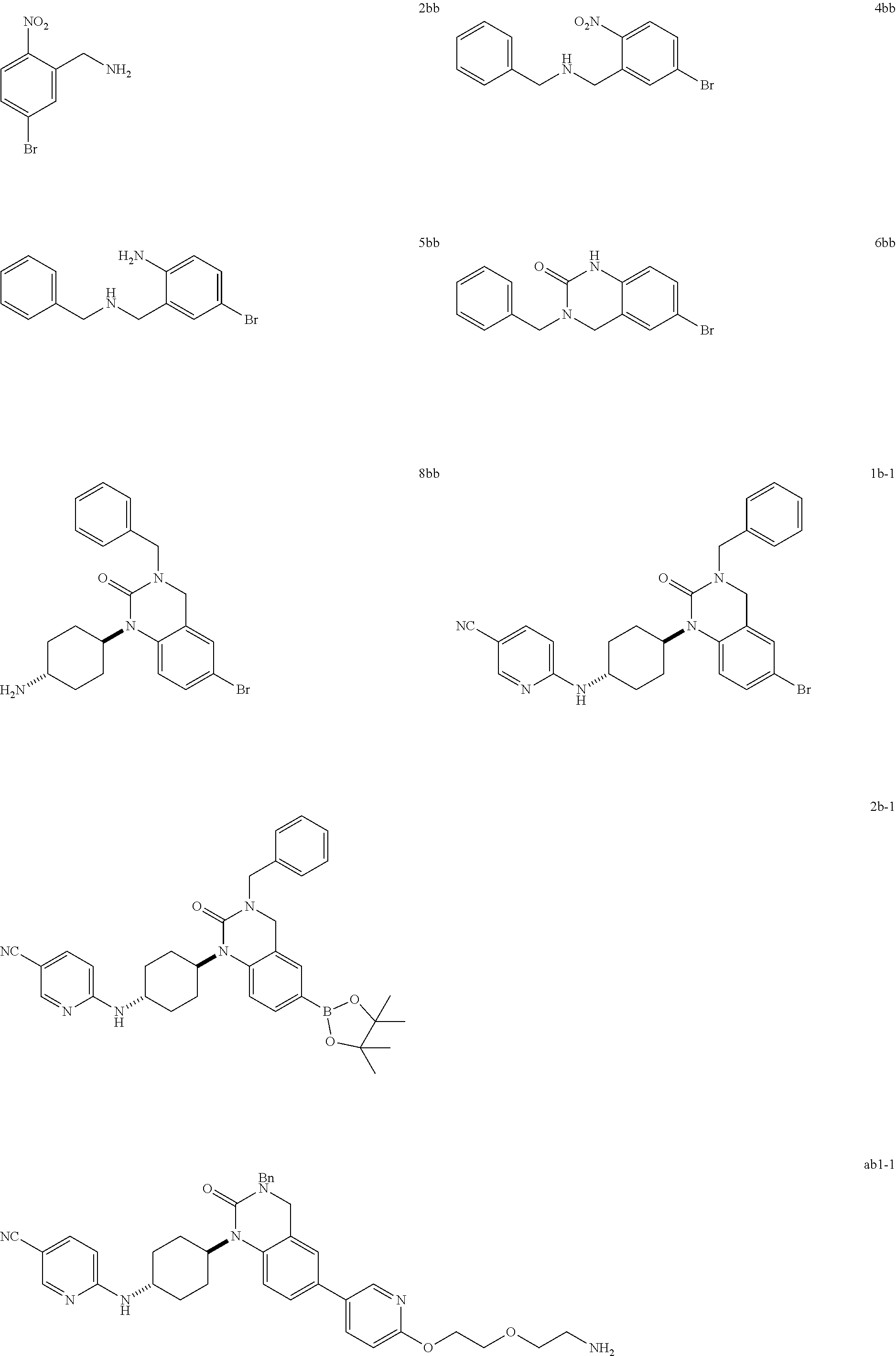

### (1) Preparation of Intermediate 2bb

In a 100 mL round-bottom flask, 2.0 g of 2-nitro-5-bromobenzonitrile (1bb) is added. After purging with nitrogen three times, slowly add 18 ml of borane-tetrahydrofuran complex (1M) dropwise in an ice bath. Purge with nitrogen three more times, and stir overnight. After TLC shows the complete consumption of the starting material, extract three times with 20 mL of ethyl acetate. Concentrate the filtrate under reduced pressure and rotary dry. Purify by silica gel column chromatography to obtain 1.1 g of compound 2bb, with a yield of 54%.

LC-MS (ESI) m/z (M+H)⁺ calculated for C₇H₈BrN₂O2⁺=230.9; found 230.9.

¹H NMR (400 MHz, Chloroform-d) δ 7.84-7.77 (m, 1H), 7.54 (t, J=1.8 Hz, 1H), 7.49 (dd, J=8.7, 2.2 Hz, 1H), 3.02 (s, 2H), 1.53 (s, 2H).

### (2) Preparation of Intermediate 4bb

In a 100 mL round-bottom flask, 460 mg of benzaldehyde (3bb) is dissolved in anhydrous tetrahydrofuran. Dissolve 1.0 g of intermediate 2bb in anhydrous tetrahydrofuran and add it dropwise to the round-bottom flask in an ice bath. After purging with nitrogen three times, stir at room temperature for 1 h. Add 545 mg of sodium cyanoborohydride and stir overnight.

After TLC shows the complete consumption of the starting material, add 20 mL of ice water to quench the reaction. Extract three times with 20 ml of ethyl acetate. Concentrate the filtrate under reduced pressure and rotary dry. Purify by silica gel column chromatography to obtain 1.2 g of compound 4bb, with a yield of 92%.

LC-MS (ESI) m/z (M+H)⁺ calculated for C₁₄H₁₄BrN₂O₂⁺=321.0; found 321.0.

¹H NMR (400 MHz, Chloroform-d) δ 7.55 (d, J=2.1 Hz, 1H), 7.49 (dd, J=8.7, 2.2 Hz, 1H), 7.35-7.28 (m, 4H), 7.25 (d, J=4.8 Hz, 1H), 3.81 (s, 2H), 3.09 (t, J=7.1 Hz, 2H), 1.49 (m, 1H).

### (3) Preparation of Intermediate 5bb

In a 100 mL round-bottom flask, 1.2 g of intermediate 4bb is dissolved in anhydrous ethyl acetate. Add 2.4 g of tin(II) chloride dihydrate. After purging with nitrogen three times, heat to 80 °C and stir for 3 h.

After TLC shows the complete consumption of the starting material, add 20 ml of ice water. Extract three times with 20 ml of ethyl acetate. Concentrate the filtrate under reduced pressure and rotary dry. Purify by silica gel column chromatography to obtain 840 mg of compound 5bb, with a yield of 77%.

LC-MS (ESI) m/z (M+H)⁺ calculated for C₁₄H₁₆BrN₂⁺=291.0; found 291.1.

¹H NMR (400 MHz, DMSO-d₆) δ 7.57-7.50 (m, 2H), 7.47-7.42 (m, 3H), 7.15-7.08 (m, 2H), 6.63 (d, J=9.2 Hz, 1H), 4.15 (s, 2H), 3.04 (dd, J=9.8, 6.1 Hz, 2H), 1.32-1.16 (m, 1H).

### (4) Preparation of Intermediate 6bb

In a 100 mL round-bottom flask, 840 mg of intermediate 5bb is dissolved in anhydrous tetrahydrofuran solution. Add 447 mg of CDI (1,1′-carbonyldiimidazole). After stirring at room temperature for 3 h, heat to 60 °C and continue stirring for 12 h. After TLC shows the complete consumption of the starting material, extract three times with 20 mL of ethyl acetate. Concentrate the filtrate under reduced pressure and rotary dry. Purify by silica gel column chromatography to obtain 706 mg of compound 6bb, with a yield of 76%.

LC-MS (ESI) m/z (M+H)⁺ calculated for C₁₅H₁₄BrN₂O⁺=317.0; found 317.0.

¹H NMR (400 MHz, DMSO-d₆) δ 9.46 (s, 1H), 7.40-7.20 (m, 7H), 6.75 (d, J=8.2 Hz, 1H), 4.52 (s, 2H), 4.35 (s, 2H).

### (5) Preparation of Intermediate 8bb

In a 100 mL round-bottom flask, 700 mg of intermediate 6bb is dissolved in anhydrous dioxane solution. Add 97 mg of sodium hydride in portions in an ice bath. After stirring at 0 °C for 0.5 h, slowly add dropwise to the reaction system 1.2 g of cis-4-((tert-butoxycarbonyl)amino)cyclohexyl 4-methylbenzenesulfonate (7bb) dissolved in anhydrous dioxane. Heat to 60 °C and continue stirring for 8 h.

After TLC shows the complete consumption of the starting material, add 20 ml of ice water to quench the reaction. Extract three times with 20 ml of ethyl acetate. Concentrate the filtrate under reduced pressure and rotary dry. After purification by silica gel column chromatography, treat with a 2N hydrogen chloride-ethyl acetate solution. After TLC shows the complete consumption of the starting material, distill the reaction solvent under reduced pressure to obtain 572 mg of compound 8bb, with a yield of 63%.

LC-MS (ESI) m/z (M+H)⁺ calculated for C₂₁H₂₅BrN₃O⁺=414.1; found 414.1.

### (6) Preparation of Intermediate 1b-1

The preparation of intermediate 1b-1 is by reference to the preparation of Formula 1a, with the starting materials being intermediate 8bb and 6-fluoronicotinonitrile.

LC-MS (ESI) m/z (M+H)⁺ calculated for C₂₇H₂₇BrN₅O⁺=516.1; found 516.1.

### (7) Preparation of Intermediate 2b-1

The preparation of intermediate 2b-1 is by reference to the preparation of Formula 2a, with the starting materials being intermediate 1b and pinacol boronate.

LC-MS (ESI) m/z (M+H)⁺ calculated for C₃₃H₃₉BrN₅O₃⁺=564.3; found 564.3.

### (8) Preparation of the Compound as Shown in Formula Ab1-1

By reference to the preparation of Formula a0-1, with the starting materials being the compound as shown in Formula 2b and the compound as shown in Formula 5a-1.

LC-MS (ESI) m/z (M+H)⁺ calculated for C₃₆H₄₀BrN₇O₃⁺=618.3; found 618.3.

LC-MS (ESI) m/z (M+H)⁺ calculated for C₃₅H₄₀N₇O₃⁺=606.3; found 606.4.

The preparation of other intermediates (ab1-0, ab1-2, ab1-3, ab1-4, and ab1-5) is by reference to ab1-1:
ab1-0 (m₁=0, m₂=0): the starting material is replaced with 5a-0 instead of 5a-1;
ab1-2 (m₁=2, m₂=0): the starting material is replaced with 5a-2 instead of 5a-1;
ab1-3 (m₁=3, m₂=0): the starting material is replaced with 5a-3 instead of 5a-1;
ab1-4 (m₁=4, m₂=0): the starting material is replaced with 5a-4 instead of 5a-1;
ab1-5 (m₁=5, m₂=0): the starting material is replaced with 5a-5 instead of 5a-1.

The specific synthesis process for the CDK12/13 ligand terminal derivative (as shown in Formula c) (taking c0-1 as an example (where R¹ is benzyl, n=0, m₁=1, and m₂=0)) is as follows:
Coupling 3-benzyl-1-(4-bromophenyl)-1-((1r,4r)-4-((5-cyanopyridin-2-yl)amino)cyclohexyl)urea (as shown in Formula 1a) with tert-butyl piperazine-1-carboxylate (as shown in Formula 1c) to obtain 4-(4-{[(benzylamino)carbonyl]{4-[(5-cyanopyridin-2-yl)amino]cyclohexyl}amino}phenyl)piperazine-1-carboxylic acid 2-methylprop-2-yl ester, and after Boc deprotection, to obtain 1-(benzylamino)-N-{4-[(5-cyanopyridin-2-yl)amino]cyclohexyl}-N-[4-(piperazin-1-yl)phenyl]methanamide (as shown in Formula 3c);
Subjecting 2-(2-Boc-aminoethoxy)ethanol (as shown in Formula 4a-1) to a substitution reaction with p-toluenesulfonyl chloride to obtain 2-(2-((tert-butoxycarbonyl)amino)ethoxy)ethyl 4-methylbenzenesulfonate (as shown in Formula 2c-1); Subjecting 1-(benzylamino)-N-{4-[(5-cyanopyridin-2-yl)amino]cyclohexyl}-N-[4-(piperazin-1-yl)phenyl]methanamide and 2-(2-((tert-butoxycarbonyl)amino)ethoxy)ethyl 4-methylbenzenesulfonate to a nucleophilic reaction, and finally removing the protecting group under acidic conditions, to obtain the compound as shown in Formula c0-1.

When n is 0, the specific preparation process (taking c0-1 as an example (where R¹ is benzyl, n=0, m₁=1, and m₂=0)) is as follows:

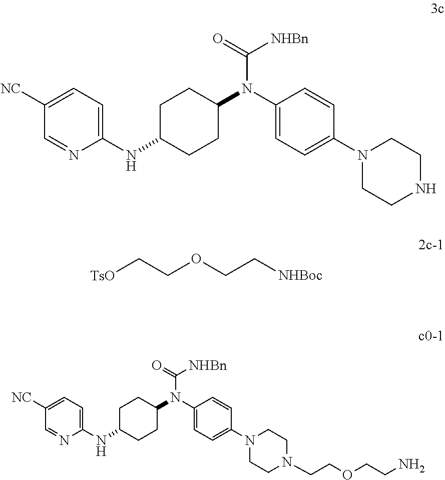

### (1) Preparation of Intermediate 3c:

In a 25 mL round-bottom flask, 200 mg of intermediate 1a, 110 mg of tert-butyl piperazine-1-carboxylate, 150 mg of sodium tert-butoxide, and 23 mg of Xantphos are added and dissolved in 5 ml of toluene solvent. After purging with nitrogen three times, 22 mg of Pd₂(dba)₃ is added. Purge with nitrogen three more times, heat to 80 °C, and stir for 2 hours.

After TLC shows the complete consumption of the starting material, concentrate the filtrate under reduced pressure and rotary dry. After purification by silica gel column chromatography, 15 mg is obtained. Treat with a 2N hydrogen chloride-ethyl acetate solution to obtain compound 3c, with a yield of 48%.

LC-MS (ESI) m/z (M+H)⁺ calculated for C₃₀H₃₆N₇O⁺=510.3; found 510.3.

¹H NMR (400 MHz, Chloroform-d) δ 8.25 (s, 1H), 7.30-7.13 (m, 6H), 7.03 (d, J=8.8 Hz, 2H), 6.89 (d, J=8.9 Hz, 2H), 6.23 (d, J=8.8 Hz, 1H), 5.21 (d, J=8.0 Hz, 1H), 4.59-4.48 (m, 1H), 4.41-4.32 (m, 3H), 3.57 (t, J=5.2 Hz, 4H), 3.48 (m, J=19.3 Hz, 1H), 3.16 (t, J=5.1 Hz, 4H), 2.05 (d, J=12.5 Hz, 2H), 1.92 (d, J=12.1 Hz, 2H), 1.44-1.28 (m, 4H).

(2) Preparation of Intermediate 2c (taking 2c-1 as an example (where m₁=1 and m₂=0)):

In a 100 mL round-bottom flask, add 1.0 μg of 4a-1, 743 mg of triethylamine, and 1 g of 4-toluenesulfonyl chloride, dissolved in dichloromethane, and stir at room temperature for 3 hours.

After TLC shows the complete consumption of the starting material, wash twice with a saturated saline bath and separate the organic phase. Concentrate the organic phase under reduced pressure, vacuum dry, and rotary dry. After purification by silica gel column chromatography, 1.7 g of compound 2c-1 is obtained, with a yield of 98%.

LC-MS (ESI) m/z (M+H)⁺ calculated for C₁₆H₂₆NO₆S⁺=360.2; found 360.3.

¹H NMR (400 MHz, Chloroform-d) δ=7.80 (d, J=8.7 Hz, 2H), 7.36 (d, J=8.7 Hz, 2H), 4.18-4.15 (m, 2H), 3.63 (t, J=4.6 Hz, 2H), 3.45 (t, J=5.0 Hz, 2H), 3.24 (q, J=5.0 Hz, 2H), 2.45 (s, 3H), 1.45 (s, 9H). 13C NMR (100 MHz, Chloroform-d) δ=156.24, 144.98, 133.09, 129.91, 128.01, 79.36, 70.40, 69.18, 68.41, 40.29, 28.46, 21.68

The preparation of other intermediates (2c-0, 2c-2, 2c-3, 2c-4, and 2c-5) is by reference to 2c-1:
2c-0 (m₁=0, m₂=0): the starting material is replaced with 4a-0 instead of 4a-1;
2c-2 (m₁=2, m₂=0): the starting material is replaced with 4a-2 instead of 4a-1;
2c-3 (m₁=3, m₂=0): the starting material is replaced with 4a-3 instead of 4a-1;
2c-4 (m₁=4, m₂=0: the starting material is replaced with 4a-4 instead of 4a-1;
2c-5 (m₁=5, m₂=0): the starting material is replaced with 4a-5 instead of 4a-1.
   (3) Preparation of the Compound as Shown in Formula c0-1:

In a 25 mL round-bottom flask, 100 mg of intermediate 1c, 141 mg of intermediate 2c-1, and 30 mg of triethylamine are added and dissolved in anhydrous acetonitrile solvent. Heat to 60 °C and stir for 8 hours. After TLC shows the complete consumption of the starting material, wash twice with a saturated saline bath and separate the organic phase. Concentrate the organic phase under reduced pressure, vacuum dry, and rotary dry. After purification by silica gel column chromatography, 86 mg is obtained. Treat with a 2N hydrogen chloride-ethyl acetate solution to obtain compound c0-1, with a yield of 63%.

LC-MS (ESI) m/z (M+H)⁺ calculated for C₃₄H₄₅N₈O₂_{⁺}=597.4; found 597.5.

The preparation of other intermediates (c0-0, c0-2, c0-3, c0-4, and c0-5) is by reference to c0-1:
c0-0 (m₁=0, m₂=0): the starting material is replaced with 2c-0 instead of 4a-1;
c0-2 (m₁=2, m₂=0): the starting material is replaced with 2c-2 instead of 4a-1;
c0-3 (m₁=3, m₂=0): the starting material is replaced with 2c-3 instead of 4a-1;
c0-4 (m₁=4, m₂=0): the starting material is replaced with 2c-4 instead of 4a-1;
c0-5 (m₁=5, m₂=0): the starting material is replaced with 2c-5 instead of 4a-1.

When n is not 0, the specific preparation process (taking cb1-1 as an example (where R¹ is benzyl, n=1, m₁=1, and m₂=0)) is as follows:

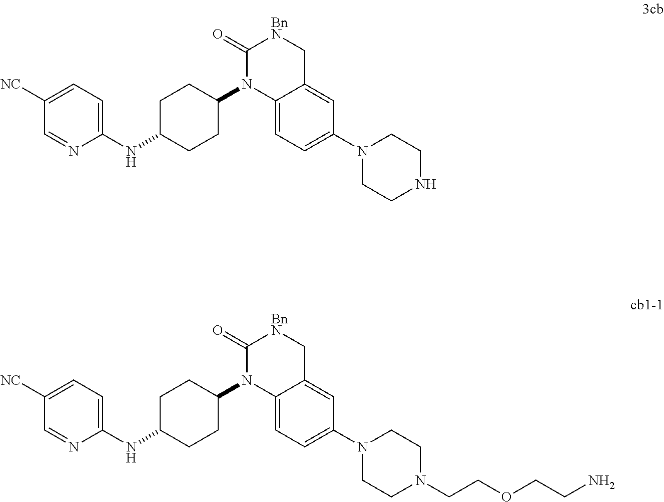

### (1) Preparation of the Compound as Shown in Formula c-1:

The preparation of intermediate 3cb is by reference to the preparation of Formula 3c, with the starting materials being the compound as shown in Formula 1b and tert-butyl piperazine-1-carboxylate (as shown in Formula 1c).

LC-MS (ESI) m/z (M+H)⁺ calculated for C₃₁H₃₆N₇O⁺=522.3; found 522.3.

### (2) Preparation of the Compound as Shown in Formula Cb1-1:

By reference to the preparation of Formula c0-1, with the starting materials being the compound as shown in Formula 3cb and the compound as shown in Formula 2c-1.

LC-MS (ESI) m/z (M+H)⁺ calculated for C₃₅H₄₅N₈O₂⁺=609.4; found 609.4.

The preparation of other intermediates (cb1-0, cb1-2, cb1-3, cb1-4, and cb1-5) is by reference to cb1-1:
cb1-0 (m₁=0, m₂=0): the starting material is replaced with 2c-0 instead of 2c-1;
cb1-2 (m₁=2, m₂=0): the starting material is replaced with 2c-2 instead of 2c-1;
cb1-3 (m₁=3, m₂=0): the starting material is replaced with 2c-3 instead of 2c-1;
cb1-4 (m₁=4, m₂=0): the starting material is replaced with 2c-4 instead of 2c-1;
cb1-5 (m₁=5, m₂=0): the starting material is replaced with 2c-5 instead of 2c-1.

The specific synthesis process for the CDK12/13 ligand terminal derivative (as shown in Formula d) (taking d0-1 as an example (where R¹ is benzyl, n=0, m₁=1, and m₂=0)) is as follows:
1) A nucleophilic substitution reaction between tert-butyl piperazine-1-carboxylate (as shown in Formula 1c) and 5-bromo-2-fluoropyridine (as shown in Formula 3a) is performed to obtain 4-(5-bromopyridin-2-yl)piperazine-1-carboxylic acid tert-butyl ester (as shown in Formula 1d); 2) 4-(5-bromopyridin-2-yl)piperazine-1-carboxylic acid tert-butyl ester is subjected to a coupling reaction with intermediate 2a, and the protecting group is removed under acidic conditions to obtain 1-(benzylamino)-N-{4-[(5-cyanopyridin-2-yl)amino]cyclohexyl}-N-{4-[6-(piperazin-1-yl)pyridin-3-yl]phenyl}methanamide (as shown in Formula 2d); 3) The 1-(benzylamino)-N-{4-[(5-cyanopyridin-2-yl)amino]cyclohexyl}-N-{4-[6-(piperazin-1-yl)pyridin-3-yl]phenyl}methanamide is subjected to a nucleophilic reaction with 2-(2-((tert-butoxycarbonyl)amino)ethoxy)ethyl 4-methylbenzenesulfonate (as shown in Formula 2c-1) to obtain the compound as shown in Formula d0-1.

When n is 0, the specific preparation process (taking d0-1 as an example, where R¹ is benzyl, n=0, m₁=1, and m₂=0) is as follows:

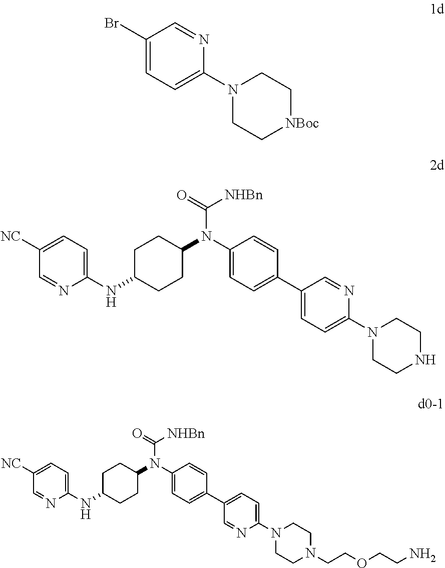

### (1) Preparation of Intermediate 1d:

In a 50 mL round-bottom flask, 200 mg of tert-butyl piperazine-1-carboxylate, 227 mg of 2-fluoro-5-bromopyridine, and 416 mg of DIPEA are added and dissolved in anhydrous dimethyl sulfoxide solution. Heat to 120 °C and stir for 3 hours.

After TLC shows the complete consumption of the starting material, wash twice with a saturated saline bath and separate the organic phase. Concentrate the organic phase under reduced pressure, vacuum dry, and rotary dry. After purification by silica gel column chromatography, 347 mg of compound 1d is obtained, with a yield of 94%.

LC-MS (ESI) m/z (M+H)⁺ calculated for C₁₄H₂₁BrN₃O₂⁺=342.1; found 342.1.

¹H NMR (400 MHz, Chloroform-d) δ=8.21 (d, J=2.4 Hz, 1H), 7.56 (dd, J=9.2, 2.8 Hz, 1H), 6.56 (d, J=4.0 Hz, 1H), 3.51-3.56 (m, 8H), 1.64 (s, 9H).

### (2) Preparation of Intermediate 2d

In a 25 mL round-bottom flask, add 80 mg of intermediate 2a, 60 mg of intermediate 1d, and 142 mg of cesium carbonate, dissolved in a mixed solvent of toluene:ethanol:water=4:4:1. After purging with nitrogen three times, add 16 mg of Pd(PPh₃)₄. Purge with nitrogen three more times, heat to 100 °C, and stir for 3 hours.

After TLC shows the complete consumption of the starting material, extract three times with 20 mL of dichloromethane. Concentrate the organic phase under reduced pressure and rotary dry. Purify by silica gel column chromatography to obtain 77 mg, and treat with a 2N hydrogen chloride-ethyl acetate solution to obtain compound 2d, with a yield of 78%.

LC-MS (ESI) m/z (M+H)⁺ calculated for C₃₅H₃₉N₈O⁺=586.3; found 586.3.

(3) Preparation of the Compound as Shown in Formula d0-1:

In a 25 mL round-bottom flask, 60 mg of intermediate 2d, 44 mg of intermediate 2c-1, and 31 mg of triethylamine are added and dissolved in anhydrous acetonitrile solvent. Heat to 60 °C and stir for 8 hours.

After TLC shows the complete consumption of the starting material, wash twice with a saturated saline bath and separate the organic phase. Concentrate the organic phase under reduced pressure, vacuum dry, and rotary dry. After purification by silica gel column chromatography, 50 mg is obtained. Treat with a 2N hydrogen chloride-ethyl acetate solution to obtain compound d0-1, with a yield of 63%.

LC-MS (ESI) m/z (M+H)+ calculated for C₃₉H₄₈N₉O₂⁺=674.4; found 674.5.

The preparation of other intermediates (d0-0, d0-2, d0-3, d0-4, and d0-5) is by reference to d0-1:
d0-0 (m₁=0, m₂=0): the starting material is replaced with 2c-0 instead of 2c-1;
d0-2 (m₁=2, m₂=0): the starting material is replaced with 2c-2 instead of 2c-1;
d0-3 (m₁=3, m₂=0): the starting material is replaced with 2c-3 instead of 2c-1;
d0-4 (m₁=4, m₂=0): the starting material is replaced with 2c-4 instead of 2c-1;
d0-5 (m₁=5, m₂=0): the starting material is replaced with 2c-5 instead of 2c-1.

When n is not 0, the specific preparation process (taking db1-1 as an example, where R¹ is benzyl, n=1, m₁=1, and m₂=0) is as follows:

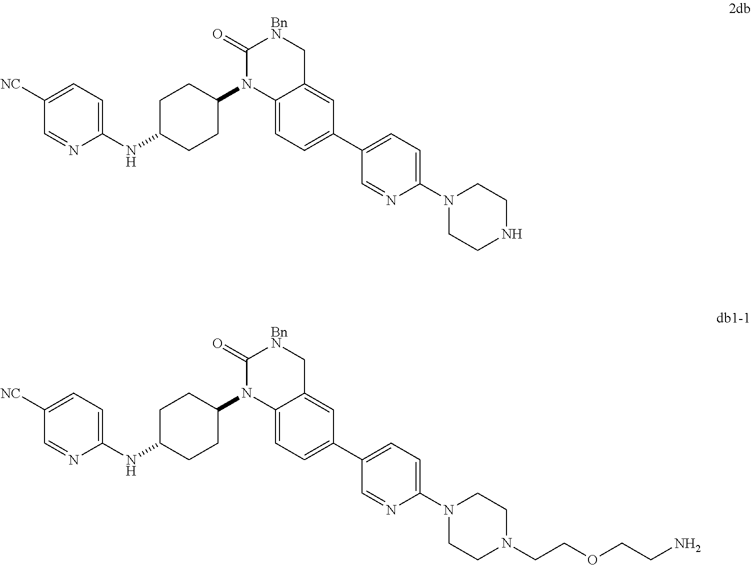

(1) The preparation of intermediate 2db is by reference to the preparation of Formula 2d, with the starting materials being the compound as shown in Formula 2b and tert-butyl piperazine-1-carboxylate (as shown in Formula 1c).

LC-MS (ESI) m/z (M+H)⁺ calculated for C₃₆H₃₉N₈O⁺=599.3; found 599.3.

(2) By reference to the preparation of Formula d0-1, with the starting materials being the compound as shown in Formula 2db and the compound as shown in Formula 2c-1.

LC-MS (ESI) m/z (M+H)⁺ calculated for C₄₀H₄₈N₉O₂⁺=686.4; found 686.5.

The preparation of other intermediates (db1-0, db1-2, db1-3, db1-4, and db1-5) is by reference to db1-1:
db1-0 (m₁=0, m₂=0): the starting material is replaced with 2c-0 instead of 2c-1;
db1-2 (m₁=2, m₂=0): the starting material is replaced with 2c-2 instead of 2c-1;
db1-3 (m₁=3, m₂=0): the starting material is replaced with 2c-3 instead of 2c-1;
db1-4 (m₁=4, m₂=0): the starting material is replaced with 2c-4 instead of 2c-1;
db1-5 (m₁=5, m₂=0): the starting material is replaced with 2c-5 instead of 2c-1.

The specific synthesis process for the CDK12/13 ligand terminal derivative (as shown in Formula e) (taking e0-1 as an example, where R¹ is benzyl, n=0, m₁=1, and m₂=0) is as follows: 1) A nucleophilic substitution reaction between tert-butyl 2-(2-hydroxyethoxy)acetate and 5-bromo-2-fluoropyridine is performed to obtain (2-((5-bromopyridin-2-yl)oxy)ethoxy)acetic acid tert-butyl ester (as shown in Formula 1e-1); 2) The (2-((5-bromopyridin-2-yl)oxy)ethoxy)acetic acid tert-butyl ester is then coupled with intermediate 2a, and the protecting group is finally removed under acidic conditions, to obtain the compound as shown in Formula e0-1.

When n is 0, the specific preparation process (taking e0-1 as an example, where R¹ is benzyl, n=0, m₁=1, and m₂=0) is as follows:

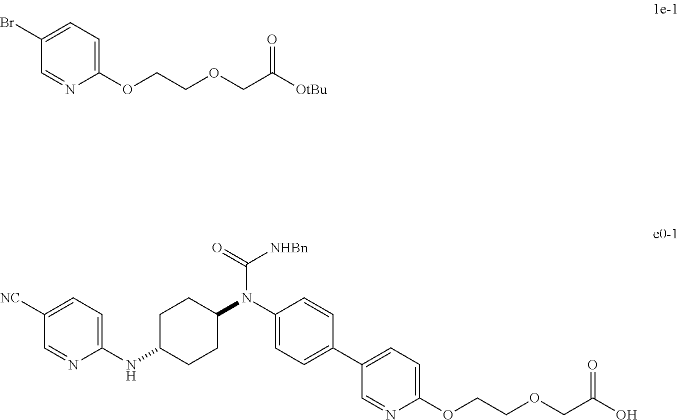

(1) Preparation of Intermediate 1e (Taking 1e-1 as an Example (where m₁=1 and m₂=0)):

In a 100 mL round-bottom flask, 860 mg of intermediate tert-butyl 2-(2-hydroxyethoxy)acetate, 1.1 g of 2-fluoro-5-bromopyridine, and 4.8 g of cesium carbonate are added and dissolved in anhydrous dimethyl sulfoxide. Heat the reaction system to 100 °C and stir for 6 hours.

After TLC shows the complete consumption of the starting material, pour the reaction mixture into 100 ml of saturated saline. Extract three times with 60 ml of ethyl acetate. Concentrate the organic phase under reduced pressure and rotary dry. Purify by silica gel column chromatography to obtain 1.41 g of compound 1e-1, with a yield of 87%.

LC-MS (ESI) m/z (M+H)⁺ calculated for C₁₃H₁₉BrNO₄⁺=332.1; found 332.1.

The preparation of other intermediates (1e-0, 1e-2, 1e-3, 1e-4, and 1e-5) is by reference to 1e-1:
1e-0 (m₁=0, m₂=0): the starting material is replaced with tert-butyl 2-hydroxyacetate instead of tert-butyl 2-(2-hydroxyethoxy)acetate;
1e-2 (m₁=2, m₂=0): the starting material is replaced with tert-butyl 2-(2-(2-hydroxyethoxy)ethoxy)acetate instead of tert-butyl 2-(2-hydroxyethoxy)acetate;
1e-3 (m₁=3, m₂=0): the starting material is replaced with tert-butyl 2-(2-(2-(2-hydroxyethoxy)ethoxy)ethoxy)acetate instead of tert-butyl 2-(2-hydroxyethoxy)acetate;
1e-4 (m₁=4, m₂=0): the starting material is replaced with tert-butyl 14-hydroxy-3,6,9,12-tetraoxatetradecan-1-carboxylate instead of tert-butyl 2-(2-hydroxyethoxy)acetate;
1e-5 (m₁=5, m₂=0): the starting material is replaced with tert-butyl 17-hydroxy-3,6,9,12,15-pentaoxaheptadecan-1-oate instead of tert-butyl 2-(2-hydroxyethoxy)acetate.
   (2) Preparation of the Compound as Shown in Formula e0-1:

In a 25 mL round-bottom flask, 80 mg of intermediate 2a, 58 mg of intermediate 1e-1, and 142 mg of cesium carbonate are added and dissolved in a mixed solvent of toluene:ethanol:water=4:4:1. After purging with nitrogen three times, 16 mg of Pd(PPh₃)₄ is added. Purge with nitrogen three more times, heat to 100 °C, and stir for 3 hours.

After TLC shows the complete consumption of the starting material, extract three times with 20 mL of dichloromethane. Concentrate the organic phase under reduced pressure and rotary dry. Purify by silica gel column chromatography to obtain 61 mg, and treat with a 50% TFA in dichloromethane solution to obtain compound e0-1, with a yield of 62%.

LC-MS(ESI) m/z(M+H)⁺ calculated for C₃₅H₃₇N₆O₅⁺=621.3; found 621.4.

The preparation of other intermediates (e0-0, e0-2, e0-3, e0-4, and e0-5) is by reference to e0-1.
e0-0 (m₁=0, m₂=0): the starting material is replaced with 1e-0 instead of 1e-1;
e0-2 (m₁=2, m₂=0): the starting material is replaced with 1e-2 instead of 1e-1;
e0-3 (m₁=3, m₂=0): the starting material is replaced with 1e-3 instead of 1e-1;
e0-4 (m₁=4, m₂=0): the starting material is replaced with 1e-4 instead of 1e-1;
e0-5 (m₁=5, m₂=0): the starting material is replaced with 1e-5 instead of 1e-1.

When n is not 0, the specific preparation process (taking eb-1 as an example, where R¹ is benzyl, n=1, m₁=1, and m₂=0) is as follows:

### Preparation of the Compound as Shown in Formula Eb1-1:

By reference to the preparation of Formula e0-1, with the starting materials being the compound as shown in Formula 2b and the compound as shown in Formula 1e-1.

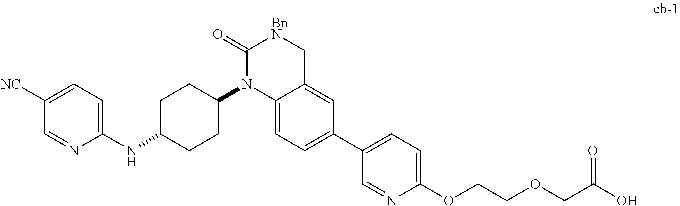

LC-MS (ESI) m/z (M+H)⁺ calculated for C₃₆H₃₇N₆O₅⁺=632.3; found 632.3.

The preparation of other intermediates (eb1-0, eb1-2, eb1-3, eb1-4, and eb1-5) is by reference to eb1-1:
eb1-0 (m₁=0, m₂=0): the starting material is replaced with 1e-0 instead of 1e-1;
eb1-2 (m₁=2, m₂=0): the starting material is replaced with 1e-2 instead of 1e-1;
eb1-3 (m₁=3, m₂=0): the starting material is replaced with 1e-3 instead of 1e-1;
eb1-4 (m₁=4, m₂=0): the starting material is replaced with 1e-4 instead of 1e-1;
eb1-5 (m₁=5, m₂=0): the starting material is replaced with 1e-5 instead of 1e-1.

The specific synthesis process for the CDK12/13 ligand terminal derivative (as shown in Formula g) (taking g0-1 as an example, where R¹ is benzyl, n=0, m₁=1, and m₂=0) is as follows: 1) tert-butyl 2-(2-hydroxyethoxy)acetate undergoes a substitution reaction with 4-toluenesulfonyl chloride to obtain tert-butyl 2-(2-(4-toluenesulfonyloxy)ethoxy)acetate (as shown in Formula 1g-1); 2) the tert-butyl 2-(2-(4-toluenesulfonyloxy)ethoxy)acetate then undergoes a nucleophilic reaction with intermediate 3c, and the protecting group is removed under acidic conditions to obtain the compound as shown in Formula g0-1.

When n is 0, the specific preparation process (taking g0-1 as an example, where R¹ is benzyl, n=0, m₁=1, and m₂=0) is as follows:

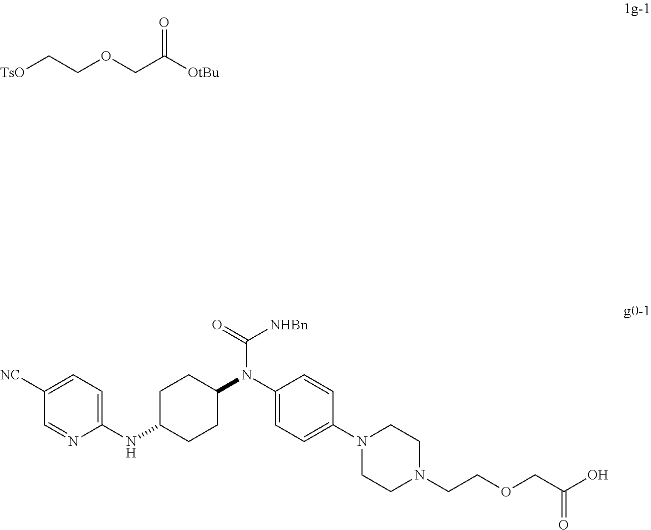

(1) Preparation of Intermediate 1 g (Taking 1 g-1 as an Example (where m₁=1 and m₂=0)):

In a 100 mL round-bottom flask, add 1.0 g of tert-butyl 2-(2-hydroxyethoxy)acetate, 861 mg of triethylamine, and 1.1 g of 4-toluenesulfonyl chloride, dissolved in dichloromethane, and stir at room temperature for 3 hours.

After TLC shows the complete consumption of the starting material, wash twice with a saturated saline bath and separate the organic phase. Concentrate the organic phase under reduced pressure, vacuum dry, and rotary dry. After purification by silica gel column chromatography, 1.8 g of compound 1g-1 is obtained, with a yield of 96%.

LC-MS (ESI) m/z (M+H)⁺ calculated for C₁₅H₂₃O₆⁺=331.1; found 331.2.

¹H NMR (400 MHz, Chloroform-d) δ=7.77-7.83 (m, 2H), 7.44 (d, J=7.83 Hz, 2H), 4.14-4.19 (m, 2H), 3.93 (s, 2H), 3.68-3.74 (m, 2H), 2.46 (s, 3H), 1.46 (s, 9H).

The preparation of other intermediates (1 g-0, 1 g-2, 1 g-3, 1 g-4, and 1 g-5 . . . ) is by reference to 1 g-1:
1 g-0 (m₁=0, m₂=0): the starting material is replaced with tert-butyl 2-hydroxyacetate instead of tert-butyl 2-(2-hydroxyethoxy)acetate;
1 g-2 (m₁=2, m₂=0): the starting material is replaced with tert-butyl 2-(2-(2-hydroxyethoxy)ethoxy)acetate instead of tert-butyl 2-(2-hydroxyethoxy)acetate;
1 g-3 (m₁=3, m₂=0): the starting material is replaced with tert-butyl 2-(2-(2-(2-hydroxyethoxy)ethoxy)ethoxy)acetate instead of tert-butyl 2-(2-hydroxyethoxy)acetate;
1 g-4 (m₁=4, m₂=0): the starting material is replaced with tert-butyl 14-hydroxy-3,6,9,12-tetraoxatetradecan-1-carboxylate instead of tert-butyl 2-(2-hydroxyethoxy)acetate;
1 g-5 (m₁=5, m₂=0): the starting material is replaced with tert-butyl 17-hydroxy-3,6,9,12,15-pentaoxaheptadecan-1-oate instead of tert-butyl 2-(2-hydroxyethoxy)acetate.
   (2) Preparation of the Compound as Shown in Formula g0-1:

In a 25 mg round-bottom flask, 100 mg of intermediate 3c, 78 mg of intermediate 1 g-1, and 30 mg of triethylamine are added and dissolved in anhydrous acetonitrile solvent. Heat to 60 °C and stir for 8 hours.

After TLC shows the complete consumption of the starting material, wash twice with a saturated saline bath and separate the organic phase. Concentrate the organic phase under reduced pressure, vacuum dry, and rotary dry. After purification by silica gel column chromatography, 76 mg is obtained. Treat with a 50% TFA in dichloromethane solution to obtain compound g0-1, with a yield of 57%.

LC-MS (ESI) m/z (M+H)⁺ calculated for C₃₄H₄₁N₇O₄⁺=612.3; found 612.3.

The preparation of other intermediates (g0-0, g0-2, g0-3, g0-4, and g0-5) is by reference to g0-1:
g0-0 (m₁=0, m₂=0): the starting material is replaced with 1 g-0 instead of 1 g-1;
g0-2 (m₁=2, m₂=0): the starting material is replaced with 1 g-2 instead of 1 g-1;
g0-3 (m₁=3, m₂=0): the starting material is replaced with 1 g-3 instead of 1 g-1;
g0-4 (m₁=4, m₂=0): the starting material is replaced with 1 g-5 instead of 1 g-1;
g0-5 (m₁=5, m₂=0): the starting material is replaced with 1 g-5 instead of 1 g-1.

When n is not 0, the specific preparation process (taking gb1-1 as an example, where R¹ is benzyl, n=1, m₁=1, and m₂=0) is as follows:

### (1) Preparation of the Compound as Shown in Formula Gb1-1:

By reference to the preparation of Formula g0-1, with the starting materials being the compound as shown in Formula 3cb and the compound as shown in Formula 1g-1.

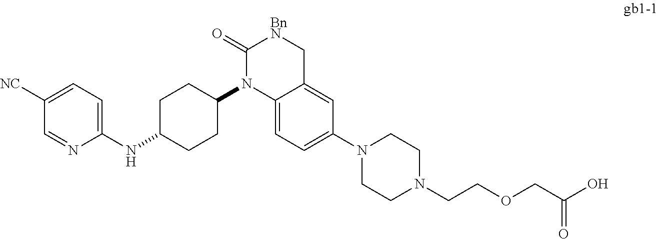

LC-MS (ESI) m/z (M+H)⁺ calculated for C₃₅H₄₂N₇O₄⁺=624.3; found 624.3.

The preparation of other intermediates (gb1-0, gb1-2, gb1-3, gb1-4, and gb1-5) is by reference to gb1-1:
gb1-0 (m₁=0, m₂=0): the starting material is replaced with 1 g-0 instead of 1 g-1;
gb1-2 (m₁=2, m₂=0): the starting material is replaced with 1 g-2 instead of 1 g-1;
gb1-3 (m₁=3, m₂=0): the starting material is replaced with 1 g-3 instead of 1 g-1;
gb1-4 (m₁=4, m₂=0): the starting material is replaced with 1 g-4 instead of 1 g-1;
gb1-5 (m₁=5, m₂=0): the starting material is replaced with 1 g-5 instead of 1 g-1.

The specific synthesis process for the CDK12/13 ligand terminal derivative (as shown in Formula h) (taking h0-1 as an example, where R1 is benzyl, n=0, and m=1) is as follows: 1) tert-butyl 4-(5-bromopyridin-2-yl)piperazine-1-carboxylate (as shown in Formula 1d) undergoes a substitution reaction with tert-butyl 2-(2-(4-toluenesulfonyloxy)ethoxy)acetate (as shown in Formula 1g-1) to obtain tert-butyl (2-(4-(5-bromopyridin-2-yl)piperazin-1-yl)ethoxy)acetate (as shown in Formula 1h-1); 2) the tert-butyl (2-(4-(5-bromopyridin-2-yl)piperazin-1-yl)ethoxy)acetate then undergoes a coupling reaction with intermediate 2a, and the protecting group is finally removed under acidic conditions, to obtain the compound as shown in Formula h0-1.

When n is 0, the specific preparation process (taking h0-1 as an example, where R¹ is benzyl, n=0, m₁=1, and m₂=0) is as follows:

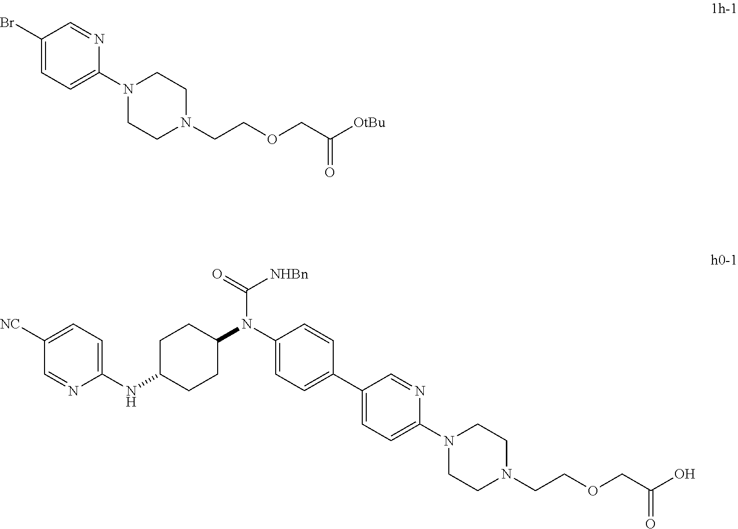

(1) Preparation of Intermediate 1h (Taking 1h-1 as an Example (where n=0, m=1)):

In a 50 mL round-bottom flask, 200 mg of intermediate 1d is added. After being treated with a 2N hydrogen chloride-ethyl acetate solution and then vacuum dried, 325 mg of intermediate 1g-1 and 125 mg of anhydrous triethylamine are added and dissolved in anhydrous acetonitrile solution. Heat to 60 °C and stir for 8 hours.

After TLC shows the complete consumption of the starting material, wash twice with a saturated saline bath and separate the organic phase. Concentrate the organic phase under reduced pressure, vacuum dry, and rotary dry. After purification by silica gel column chromatography, 268 mg of compound 1 h-1 is obtained, with a yield of 81%.

LC-MS (ESI) m/z (M+H)⁺ calculated for C₁₇H₂₆BrN₃O₃⁺=400.1; found 400.2.

The preparation of other intermediates (1h-0, 1h-2, 1h-3, 1h-4, and 1h-5) is by reference to 1h-1:
1h-0 (m₁=0, m₂=0): the starting material is replaced with 1g-0 instead of 1g-1;
1h-2 (m₁=2, m₂=0): the starting material is replaced with 1g-2 instead of 1g-1;
1h-3 (m₁=3, m₂=0): the starting material is replaced with 1g-3 instead of 1g-1;
1h-4 (m₁=4, m₂=0): the starting material is replaced with 1g-4 instead of 1g-1;
1h-5 (m₁=5, m₂=0): the starting material is replaced with 1g-5 instead of 1g-1.
   Preparation of the Compound as Shown in Formula h0-1:

In a 25 mL round-bottom flask, 80 mg of intermediate 2a, 70 mg of intermediate 1h-1, and 142 mg of cesium carbonate are added and dissolved in a mixed solvent of toluene:ethanol:water=4:4:1. After purging with nitrogen three times, 16 mg of Pd(PPh₃)₄ is added. Purge with nitrogen three more times, heat to 100 °C, and stir for 3 hours.

After TLC shows the complete consumption of the starting material, extract three times with 20 ml of dichloromethane. Concentrate the organic phase under reduced pressure and rotary dry.

Purify by silica gel column chromatography to obtain 72 mg, and treat with a 50% TFA in dichloromethane solution to obtain compound h0-1, with a yield of 67%.

LC-MS (ESI) m/z (M+H)⁺ calculated for C₃₉H₄₄N₈O₄⁺=689.4; found 689.5.

The preparation of other intermediates (h0-0, h0-2, h0-3, h0-4, and h0-5) is by reference to h0-1:
h0-0 (m₁=0, m₂=0): the starting material is replaced with 1h-0 instead of 1h-1;
h0-2 (m₁=2, m₂=0): the starting material is replaced with 1h-2 instead of 1h-1;
h0-3 (m₁=3, m₂=0): the starting material is replaced with 1h-3 instead of 1h-1;
h0-4 (m₁=4, m₂=0): the starting material is replaced with 1h-4 instead of 1h-1;
h0-5 (m₁=5, m₂=0): the starting material is replaced with 1h-5 instead of 1h-1.

When n is not 0, the specific preparation process (taking hb1-1 as an example, where R¹ is benzyl, n=1, m₁=1, and m₂=0) is as follows:

### (1) Preparation of the Compound as Shown in Formula Hb1-1:

By reference to the preparation of Formula h0-1, with the starting materials being the compound as shown in Formula 2b-1 and the compound as shown in Formula 1h-1.

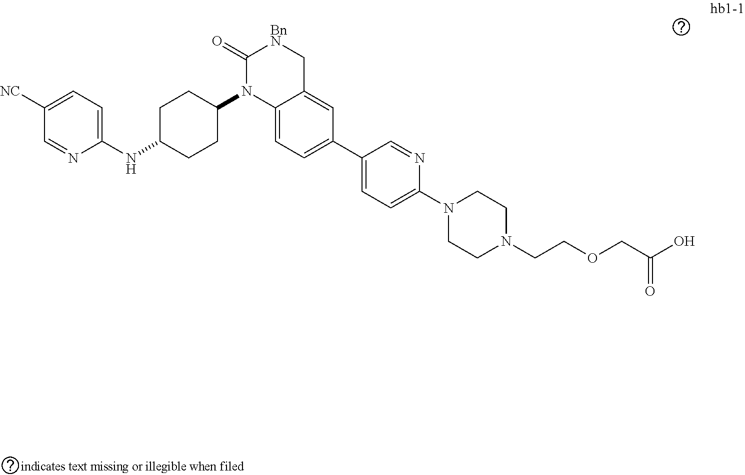

LC-MS (ESI) m/z (M+H)⁺ calculated for C₄₀H₄₅N₈O₄⁺=701.4; found 701.4.

The preparation of other intermediates (hb1-0, hb1-2, hb1-3, hb1-4, and hb1-5) is by reference to hb1-1:
hb1-0 (m₁=0, m₂=0): the starting material is replaced with 1h-0 instead of 1h-1;
hb1-2 (m₁=2, m₂=0): the starting material is replaced with 1h-2 instead of 1h-1;
hb1-3 (m₁=3, m₂=0): the starting material is replaced with 1h-3 instead of 1h-1;
hb1-4 (m₁=4, m₂=0): the starting material is replaced with 1h-4 instead of 1h-1;
hb1-5 (m₁=5, m₂=0): the starting material is replaced with 1h-5 instead of 1h-1.

### Example 1: Preparation of Compounds as Shown in Formulae 1 to 86

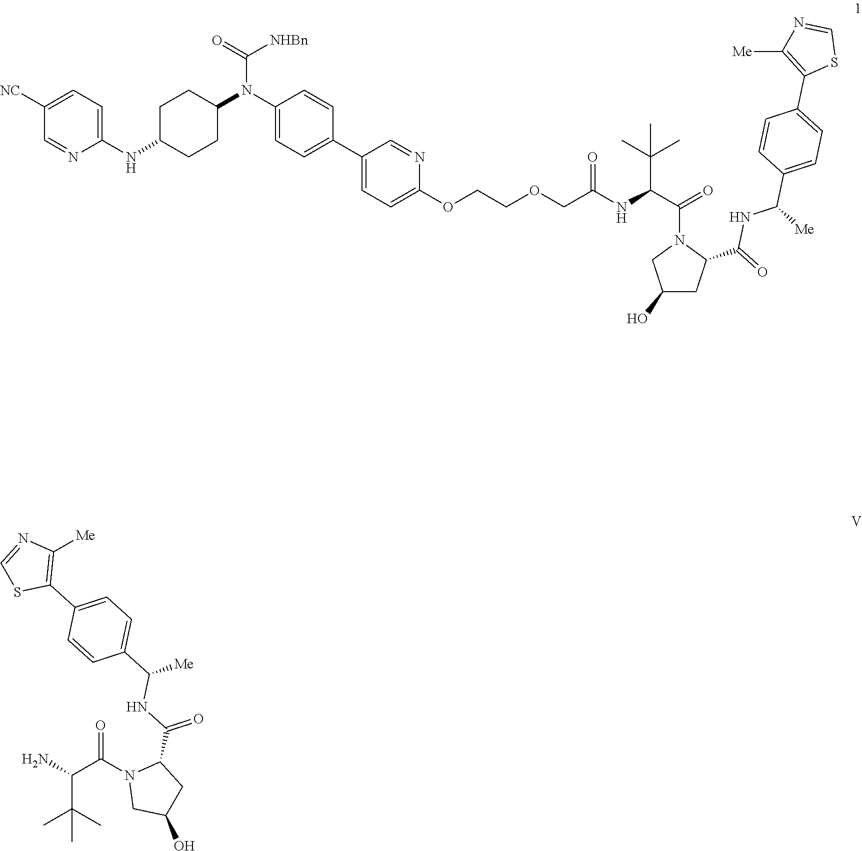

In a 10 mL round-bottom flask, 40 mg of intermediate e0-1, 29 mg of intermediate V, 37 mg of HATU, 25 mg of DIPEA, and 3 ml of DMF are added. Stir at room temperature for 20 minutes. After TLC shows the complete consumption of the starting material, wash with water and extract with ethyl acetate. Concentrate the organic phase under reduced pressure and rotary dry. Purify by silica gel column chromatography using a 20:1 (v/v) mixture of dichloromethane:methanol as the eluent to obtain the compound as shown in Formula 1, with a yield of 68%.

The preparation method for (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (Formula V) can be found in the following reference: Proc Natl Acad Sci USA. 2016 Jun. 28; 113(26):7124-9. LC-MS (ESI) m/z (M+H)⁺ calculated for C₅₈H₆₇N₁₀O₇S⁺=1047.5; found 1047.5.

¹H NMR (400 MHz, Chloroform-d) δ 8.63 (s, 1H), 8.34 (d, J=1.8 Hz, 1H), 8.26 (dd, J=2.0 Hz, 1H), 7.75 (dd, J=8.6, 2.6 Hz, 1H), 7.53 (d, J=8.4 Hz, 2H), 7.50 (d, J=7.7 Hz, 2H), 7.42 (dd, J=8.9, 2.3 Hz, 1H), 7.39-7.34 (m, 5H), 7.29-7.17 (m, 6H), 6.92 (dd, J=8.6, 0.7 Hz, 1H), 6.27 (dd, J=8.8, 0.8 Hz, 1H), 5.16-5.04 (m, 1H), 4.66-4.50 (m, 5H), 4.38 (d, J=5.8 Hz, 2H), 4.10 (d, J=2.5 Hz, 2H), 3.93 (t, J=4.2 Hz, 2H), 2.49 (m, 4H), 2.10 (d, J=13.9 Hz, 2H), 2.00 (d, J=8.4 Hz, 2H), 1.89 (s, 3H), 1.48 (d, J=6.9 Hz, 3H), 1.45-1.41 (m, 2H), 1.34-1.29 (m, 2H), 1.25-1.24 (m, 5H), 1.07 (s, 9H). 13C NMR (100 MHz, DMSO-d6) δ 170.5, 169.1, 168.5, 162.7, 159.3, 156.5, 153.1, 151.5, 147.8, 144.7, 141.3, 138.4, 137.6, 137.4, 136.4, 131.8, 131.1, 129.7, 128.9, 128.7, 128.0, 127.2, 126.8, 126.4, 126.3, 126.3, 119.2, 111.1, 109.0, 94.0, 69.5, 68.8, 64.8, 59.8, 58.6, 56.6, 55.7, 53.3, 48.6, 47.8, 43.5, 37.8, 35.9, 31.3, 30.3, 26.3, 22.5, 16.0.

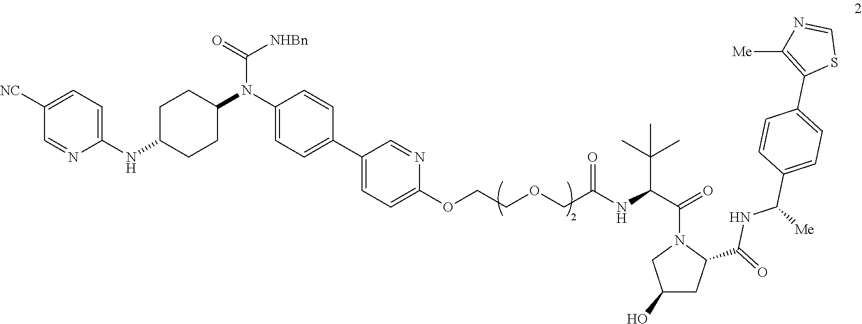

By reference to the preparation method of Formula 1, use intermediate e0-2 instead of intermediate e0-1.

LC-MS (ESI) m/z (M+H)⁺ calculated for C₆₀H₇₁N₁₀O₈S⁺=1091.5; found 1091.6.

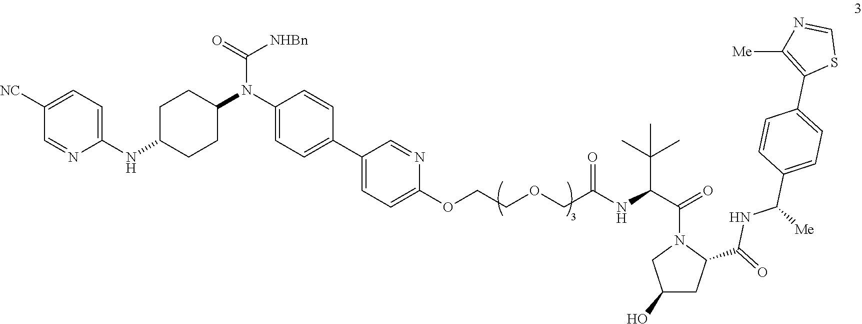

Following the preparation method for Formula 1, use intermediate e0-3 instead of intermediate e0-1.

LC-MS (ESI) m/z (M+H)⁺ calculated for C₆₂H₇₅N₁₀O₉S⁺=1135.5; found 1135.6.

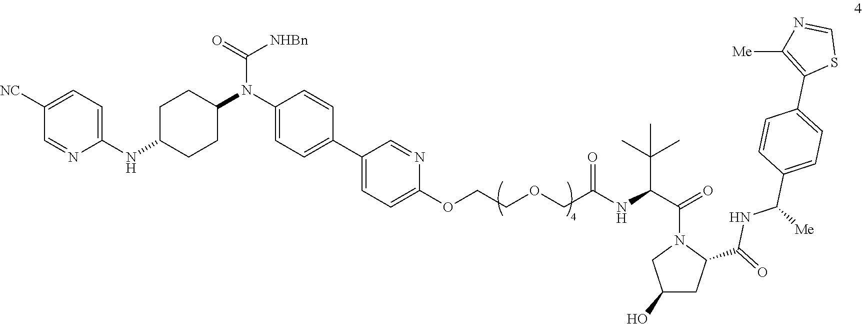

Following the preparation method for Formula 1, use intermediate e0-4 instead of intermediate e0-1.

LC-MS (ESI) m/z (M+H)⁺ calculated for C₆₄H₇₉N₁₀O₁₀S⁺=1179.6; found 1179.6.

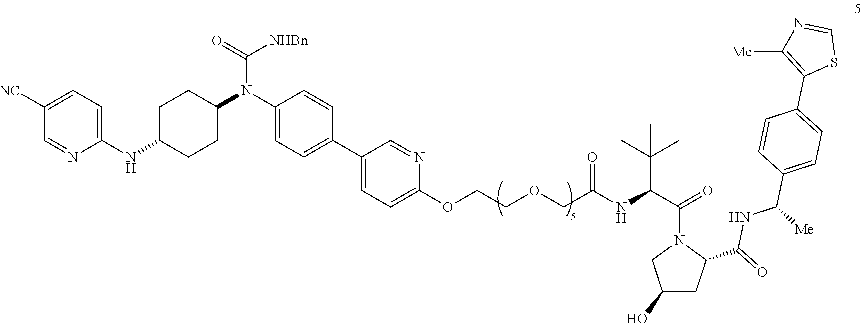

Following the preparation method for Formula 1, use intermediate e0-5 instead of intermediate e0-1.

LC-MS (ESI) m/z (M+H)⁺ calculated for C₆₆H₈₃N₁₀O₁₁S⁺=1223.6; found 1223.6.

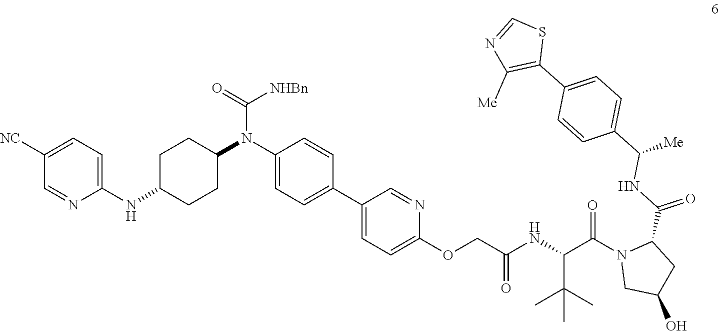

Following the preparation method for Formula 1, use intermediate e0-0 instead of intermediate e0-1.

LC-MS (ESI) m/z (M+H)⁺ calculated for C₅₆H₆₃N₁₀O₆S⁺=1003.5; found 1003.5.

¹H NMR (400 MHz, Chloroform-d) δ 8.65 (s, 1H), 8.36 (d, J=2.5 Hz, 1H), 8.26 (d, J=2.2 Hz, 1H), 7.82 (dd, J=8.6, 2.6 Hz, 1H), 7.58-7.51 (m, 2H), 7.45-7.33 (m, 6H), 7.31-7.16 (m, 6H), 7.09 (dd, J=8.8, 7.0 Hz, 1H), 6.94 (d, J=8.6 Hz, 1H), 6.27 (d, J=8.8 Hz, 1H), 5.14-5.05 (m, 2H), 4.99 (d, J=15.6 Hz, 1H), 4.82-4.64 (m, 3H), 4.54 (s, 1H), 4.39 (m, 2H), 4.09 (m, J=16.7, 9.3 Hz, 1H), 3.65 (dd, J=11.3, 3.8 Hz, 1H), 3.51 (m, J=24.5 Hz, 1H), 2.50 (s, 3H), 2.10 (d, J=12.2 Hz, 2H), 2.00 (d, J=8.9 Hz, 2H), 1.86 (m, J=14.3, 7.2 Hz, 4H), 1.48 (d, J=6.9 Hz, 3H), 1.39 (m, J=21.1 Hz, 4H), 1.01 (s, 9H).

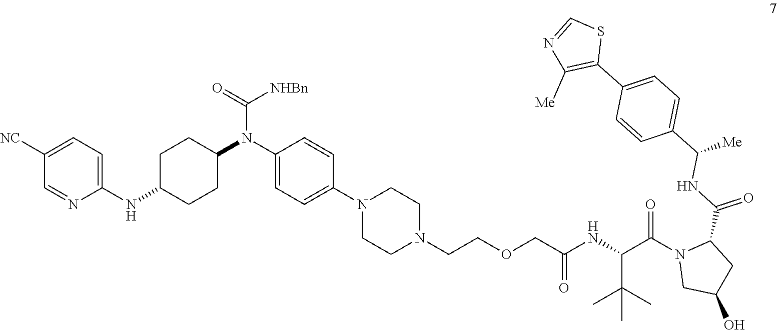

Following the preparation method for Formula 1, use intermediate g0-1 instead of intermediate e0-1.

LC-MS (ESI) m/z (M+H)⁺ calculated for C₅₇H₇₂N₁₁O₆S⁺=1038.5; found 1038.6.

¹H NMR (400 MHz, Chloroform-d) δ 8.67 (s, 1H), 8.26 (d, J=2.3 Hz, 1H), 7.50 (d, J=5.7, 3.3 Hz, 1H), 7.46 (dd, J=8.8, 2.3 Hz, 1H), 7.35 (m, J=7.6, 6.8, 4.2 Hz, 6H), 7.22-7.16 (m, 3H), 7.03-7.00 (m, 2H), 6.88 (d, J=8.7 Hz, 2H), 6.27 (d, J=8.8 Hz, 1H), 5.21 (d, J=8.1 Hz, 1H), 5.08 (t, J=7.0 Hz, 1H), 4.73 (t, J=7.8 Hz, 1H), 4.57 (d, J=8.6 Hz, 1H), 4.56-4.44 (m, 3H), 4.36 (d, J=5.9 Hz, 2H), 4.11-4.00 (m, 3H), 3.68-3.59 (m, 4H), 3.46 (s, 1H), 3.25-3.21 (m, 4H), 2.68 (m, J=6.7 Hz, 4H), 2.51 (s, 3H), 2.50-2.44 (m, 1H), 2.09 (d, J=11.7 Hz, 2H), 1.95 (d, J=12.0 Hz, 2H), 1.47 (d, J=7.0 Hz, 3H), 1.43-1.34 (m, 4H), 1.07 (s, 9H).

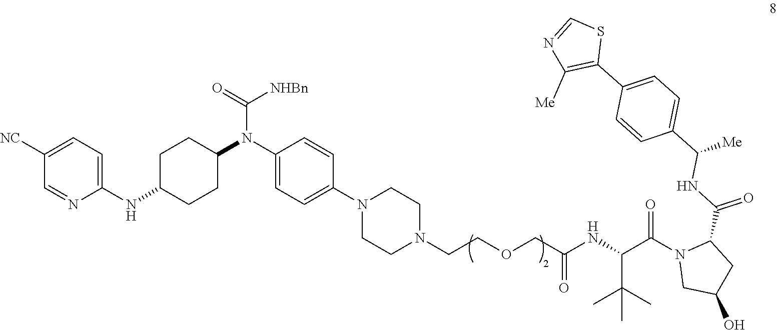

Following the preparation method for Formula 1, use intermediate g0-2 instead of intermediate e0-1.

LC-MS (ESI) m/z (M+H)⁺ calculated for C₅₉H₇₆N₁₁O₇S⁺=1082.6; found 1082.6.

¹H NMR (400 MHz, Chloroform-d) δ 8.66 (s, 1H), 8.26 (d, J=2.2 Hz, 1H), 7.45 (d, J=7.9 Hz, 1H), 7.37 (q, J=8.1 Hz, 6H), 7.25-7.14 (m, 4H), 7.00 (d, J=8.6 Hz, 2H), 6.86 (d, J=8.6 Hz, 2H), 6.25 (d, J=8.9 Hz, 1H), 5.18 (d, J=8.1 Hz, 1H), 5.07 (t, J=7.0 Hz, 1H), 4.73 (t, J=7.8 Hz, 1H), 4.57 (d, J=8.6 Hz, 1H), 4.54-4.42 (m, 3H), 4.34 (d, J=5.9 Hz, 2H), 4.11-4.00 (m, 3H), 3.73-3.57 (m, 8H), 3.47 (s, 1H), 3.25-3.21 (m, 4H), 2.68 (m, J=6.7 Hz, 4H), 2.51 (s, 3H), 2.50-2.44 (m, 1H), 2.09-1.97 (m, 4H), 1.96-1.89 (d, 2H), 1.47 (d, J=7.0 Hz, 3H), 1.43-1.34 (m, 4H), 1.09 (s, 9H).

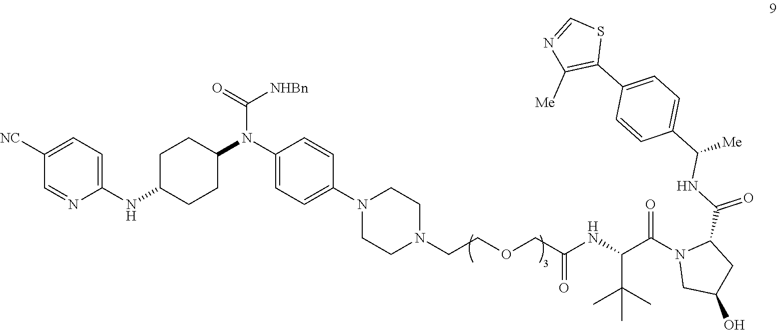

Following the preparation method for Formula 1, use intermediate g0-3 instead of intermediate e0-1.

LC-MS (ESI) m/z (M+H)⁺ calculated for C₆₁H₈₀N₁₁O₈S⁺=1126.6; found 1126.6.

¹H NMR (400 MHz, Chloroform-d) δ 8.67 (s, 1H), 8.27 (d, J=2.3 Hz, 1H), 7.53 (dd, J=5.7, 3.3 Hz, 1H), 7.46 (dd, J=8.8, 2.3 Hz, 1H), 7.37 (qd, J=7.6, 6.8, 4.2 Hz, 6H), 7.22-7.16 (m, 3H), 7.03-7.00 (m, 2H), 6.88 (d, J=8.7 Hz, 2H), 6.27 (d, J=8.8 Hz, 1H), 5.34 (t, J=3.4 Hz, 1H), 5.07 (m, J=17.4, 15.9, 8.8 Hz, 2H), 4.76 (t, J=8.0 Hz, 1H), 4.57 (d, J=8.6 Hz, 1H), 4.52-4.42 (m, 2H), 4.34 (d, J=5.9 Hz, 2H), 4.21 (t, J=6.0 Hz, 1H), 4.11-4.02 (m, 3H), 3.72-3.64 (m, 8H), 3.28 (m, 2H), 2.80 (m, 4H), 2.52 (s, 3H), 2.46 (m, 1H), 2.30 (t, J=7.4 Hz, 2H), 2.21 (t, J=7.7 Hz, 2H), 2.08 (d, J=11.7 Hz, 2H), 1.93 (d, J=12.0 Hz, 2H), 1.66 (m, J=21.9, 14.5, 7.6, 7.0 Hz, 4H), 1.48 (d, J=6.9 Hz, 3H), 1.44-1.37 (m, 4H), 1.04 (s, 9H).

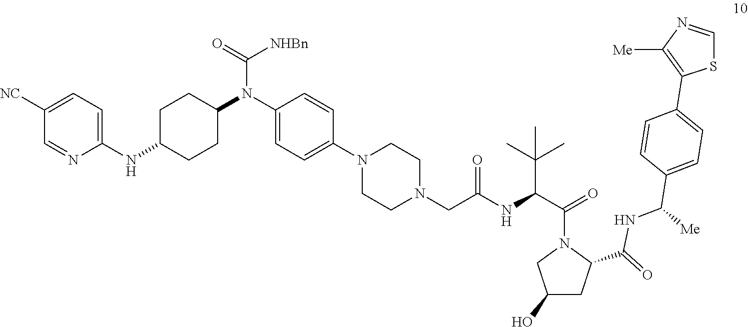

Following the preparation method for Formula 1, use intermediate g0-0 instead of intermediate e0-1.

LC-MS (ESI) m/z (M+H)⁺ calculated for C₅₅H₆₈N₁₁O₅S+=994.5; found 994.5.

¹H NMR (400 MHz, Chloroform-d) δ 8.67 (s, 1H), 8.32 (d, J=2.2 Hz, 1H), 7.50 (dd, J=5.7, 3.2 Hz, 1H), 7.45 (dd, J=8.6, 2.5 Hz, 1H), 7.39 (m, 6H), 7.20-7.14 (m, 3H), 7.03-7.00 (m, 2H), 6.92 (d, J=8.7 Hz, 2H), 6.44 (d, J=8.0 Hz, 1H), 6.27 (d, J=8.6 Hz, 1H), 5.23 (d, J=7.8 Hz, 1H), 5.11-5.05 (m, 1H), 4.76 (t, J=7.9 Hz, 1H), 4.69-4.56 (m, 1H), 4.54 (d, J=8.5 Hz, 2H), 4.48-4.42 (m, 1H), 4.40 (d, J=5.7 Hz, 2H), 4.20-4.08 (m, 2H), 3.76-3.50 (m, 7H), 3.08 (d, J=3.3 Hz, 2H), 2.68 (t, J=5.6 Hz, 4H), 2.52 (s, 3H), 2.12 (d, J=9.0 Hz, 2H), 1.99 (d, J=10.8 Hz, 2H), 1.50 (d, J=7.1 Hz, 3H), 1.38-1.30 (m, 4H), 1.08 (s, 9H).

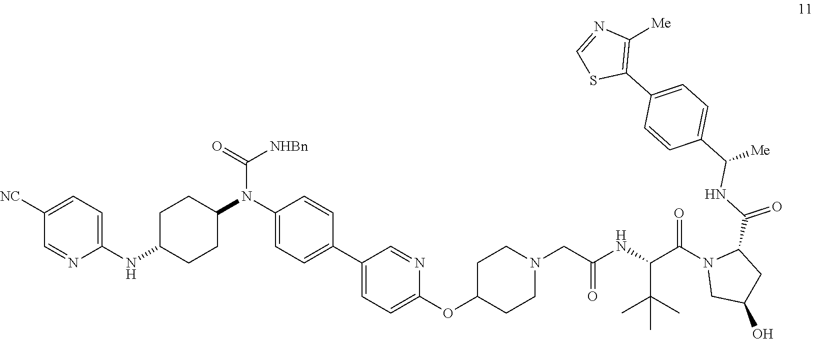

Following the preparation method for Formula 1, use intermediate e0-a instead of intermediate e0-1.

Wherein, the preparation method for intermediate e0-a is by reference to Formula e0-1, using intermediate 1e-a instead of intermediate 1e-1; and the preparation method for intermediate 1e-a is by reference to Formula 1e-1, using tert-butyl 2-(4-hydroxypiperidin-1-yl)acetate as the starting material.

LC-MS (ESI) m/z (M+H)⁺ calculated for C₆₁H₇₂N₁₁O₆S⁺=1086.5; found 1086.6.

¹H NMR (400 MHz, Chloroform-d) δ 8.64 (s, 1H), 8.32 (d, J=2.5 Hz, 1H), 8.25 (d, J=2.2 Hz, 1H), 7.76 (dd, J=8.6, 2.6 Hz, 1H), 7.54 (d, J=8.3 Hz, 2H), 7.50 (d, J=8.2 Hz, 1H), 7.41 (dd, J=8.9, 2.3 Hz, 1H), 7.39-7.33 (m, 4H), 7.30-7.15 (m, 7H), 6.80 (d, J=8.6 Hz, 1H), 6.26 (d, J=8.8 Hz, 1H), 5.19 (s, 1H), 5.14 (d, J=8.1 Hz, 1H), 5.06 (p, J=7.0 Hz, 1H), 4.75 (t, J=8.0 Hz, 1H), 4.61 (tt, J=11.6, 3.6 Hz, 1H), 4.48 (d, J=7.8 Hz, 2H), 4.41-4.35 (m, 3H), 4.12 (d, J=11.5 Hz, 1H), 3.59 (dd, J=11.4, 3.5 Hz, 1H), 3.55 (s, 1H), 3.23 (s, 1H), 2.96 (d, J=16.4 Hz, 2H), 2.63 (m, J=21.9 Hz, 2H), 2.50 (s, 3H), 2.46 (m, J=6.9 Hz, 1H), 2.14 (m, 2H), 2.09 (d, J=14.3 Hz, 2H), 1.99 (d, J=11.6 Hz, 2H), 1.47 (d, J=6.9 Hz, 3H), 1.41 (m, 5H), 1.37-1.27 (m, 4H), 1.06 (s, 9H).

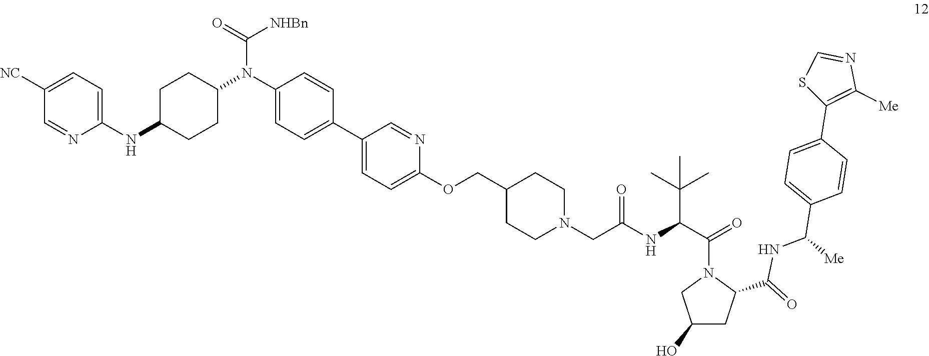

Following the preparation method for Formula 1, use intermediate e0-b instead of intermediate e0-1.

Wherein, the preparation method for intermediate e0-b is by reference to Formula e0-1, using intermediate 1e-b instead of intermediate 1e-1; and the preparation method for intermediate 1e-b is by reference to Formula 1e-1, using tert-butyl 2-(4-(hydroxymethyl)piperidin-1-yl)acetate instead of tert-butyl 2-(2-hydroxyethoxy)acetate.

LC-MS (ESI) m/z (M+H)⁺ calculated for C₆₂H₇₄N₁₁O₆S⁺=1100.6; found 1100.6.

¹H NMR (400 MHz, Chloroform-d) δ 8.65 (s, 1H), 8.36 (dd, J=2.5, 0.8 Hz, 1H), 8.27 (dd, J=2.3, 0.7 Hz, 1H), 7.78 (dd, J=8.6, 2.6 Hz, 1H), 7.55 (d, J=8.5 Hz, 2H), 7.46 (dd, J=8.9, 2.2 Hz, 2H), 7.42-7.34 (m, 5H), 7.33-7.26 (m, 2H), 7.24-7.16 (m, 4H), 6.83 (dd, J=8.6, 0.7 Hz, 1H), 6.28 (dd, J=8.9, 0.8 Hz, 1H), 5.07 (p, J=7.0 Hz, 1H), 4.93 (d, J=7.9 Hz, 1H), 4.77 (t, J=8.0 Hz, 1H), 4.67-4.57 (m, 1H), 4.51 (s, 1H), 4.45 (d, J=8.3 Hz, 1H), 4.38 (s, 3H), 4.22 (d, J=6.4 Hz, 3H), 4.16-4.07 (m, 2H), 3.59 (dd, J=11.4, 3.5 Hz, 1H), 3.09 (d, J=11.4 Hz, 1H), 2.97 (d, J=10.8 Hz, 3H), 2.52 (s, 4H), 2.33 (dd, J=18.0, 7.5 Hz, 2H), 2.11 (d, J=11.5 Hz, 3H), 2.00 (d, J=11.0 Hz, 1H), 1.91 (d, J=8.9 Hz, 2H), 1.74 (m, 7H), 1.47 (d, J=7.0 Hz, 3H), 1.43-1.36 (m, 4H), 1.07 (s, 9H).

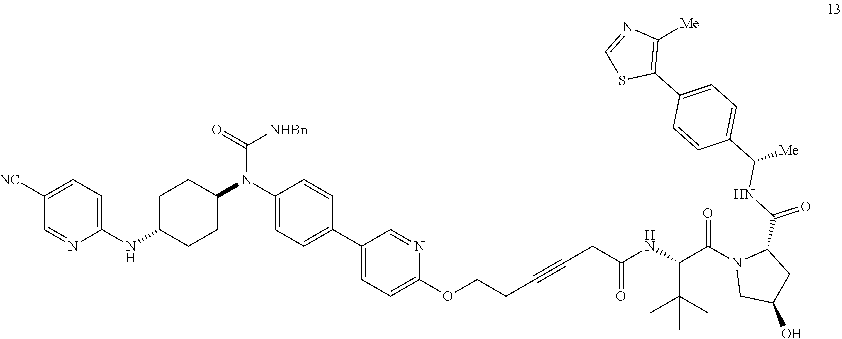

Following the preparation method for Formula 1, use intermediate e0-c instead of intermediate e0-1.

Wherein, the preparation method for intermediate e0-c is by reference to Formula e0-1, using intermediate 1e-c instead of intermediate 1e-1; and the preparation method for intermediate 1e-c is by reference to Formula 1e-1, using tert-butyl 6-hydroxyhex-3-ynoate instead of tert-butyl 2-(2-hydroxyethoxy)acetate.

LC-MS (ESI) m/z (M+H)⁺ calculated for C₆₁H₆₈N₉O₆S⁺=1054.5; found 1054.5.

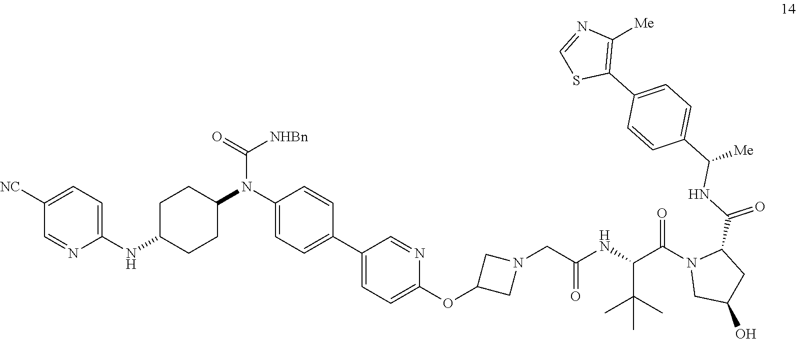

Following the preparation method for Formula 1, use intermediate e0-d instead of intermediate e0-1.

Wherein, the preparation method for intermediate e0-d is by reference to Formula e0-1, using intermediate 1e-d instead of intermediate 1e-1; and the preparation method for intermediate 1e-d is by reference to Formula 1e-1, using tert-butyl 2-(3-hydroxyazetidin-1-yl)acetate instead of tert-butyl 2-(2-hydroxyethoxy)acetate. LC-MS (ESI) m/z (M+H)⁺ calculated for C₅₉H₆₈N₁₁O₆S⁺=1058.5; found 1058.5.

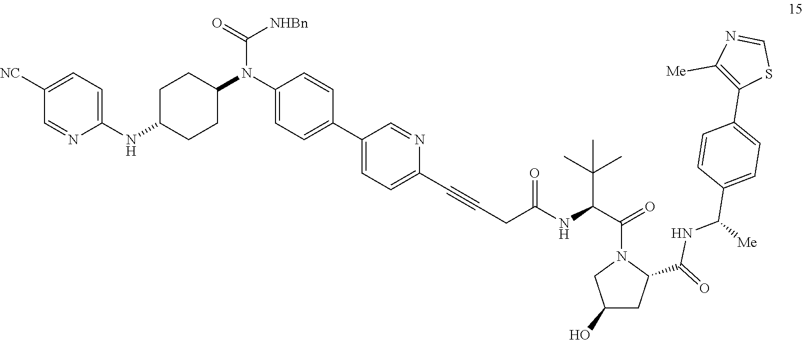

Following the preparation method for Formula 1, use intermediate e0-e instead of intermediate e0-1.

Wherein, the preparation method for intermediate e0-e is by reference to Formula e0-1, using intermediate 1e-e instead of intermediate 1e-1; and the preparation method for intermediate 1e-e is by subjecting intermediate 1a to a Sonogashira coupling reaction with tert-butyl but-3-ynoate. The specific conditions can be found in the following reference: Chemistry Letters, 2005, vol. 34, issue 9, 1294-1295.

LC-MS (ESI) m/z (M+H)⁺ calculated for C₅₈H₆₃N₁₀O₅S⁺=1011.5; found 1011.6.

¹H NMR (400 MHz, Chloroform-d) δ 8.74-8.69 (d, 1H), 8.65 (s, 1H), 8.25 (d, J=2.2 Hz, 1H), 7.80 (dd, J=8.1, 2.4 Hz, 1H), 7.62-7.56 (d, 2H), 7.46 (d, J=8.1 Hz, 1H), 7.44-7.39 (m, 2H), 7.37 (m, J=5.8 Hz, 4H), 7.30-7.16 (m, 6H), 6.59 (d, J=8.8 Hz, 1H), 6.27 (d, J=8.8 Hz, 1H), 5.14-5.03 (m, 2H), 4.73 (t, J=8.0 Hz, 1H), 4.68 (d, J=8.9 Hz, 1H), 4.61 (m, 1H), 4.53 (s, 1H), 4.37 (s, 3H), 3.62 (dd, J=11.3, 3.6 Hz, 1H), 3.53 (m, 1H), 2.88-2.69 (m, 2H), 2.62-2.51 (m, 3H), 2.50 (s, 3H) 2.11 (d, J=6.1 Hz, 2H), 2.04 (m, 5H), 1.99 (d, J=13.2 Hz, 2H), 1.47 (d, J=6.9 Hz, 3H), 1.36 (m, J=39.3, 2.9 Hz, 5H), 1.05 (s, 9H).

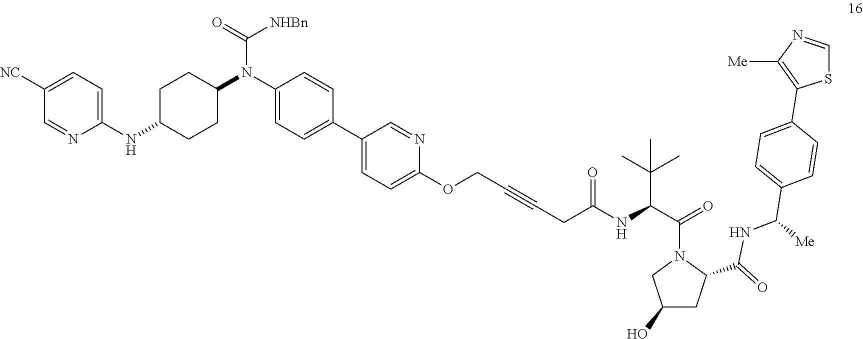

Following the preparation method for Formula 1, use intermediate e0-f instead of intermediate e0-1.

Wherein, the preparation method for intermediate e0-f is by reference to Formula e0-1, using intermediate 1e-f instead of intermediate 1e-1; and the preparation method for intermediate 1e-f is by reference to Formula 1e-1, using tert-butyl 5-hydroxypent-3-isocyanoate instead of tert-butyl 2-(2-hydroxyethoxy)acetate.

LC-MS (ESI) m/z (M+H)⁺ calculated for C₅₉H₆₅N₁₀O₆S⁺=1041.5; found 1041.6.

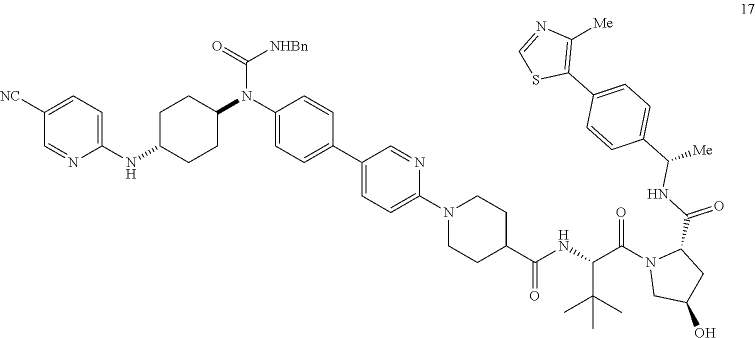

Following the preparation method for Formula 1, use intermediate h0-a instead of intermediate e0-1.

Wherein, the preparation method for intermediate h0-a is by reference to Formula h0-1, using intermediate 1h-a instead of intermediate 1h-1; and the preparation method for intermediate 1h-a is by reference to Formula 1d, using tert-butyl piperidine-4-carboxylate instead of tert-butyl piperazine-1-carboxylate.

LC-MS (ESI) m/z (M+H)⁺ calculated for C₆₀H₇₀N₁₁O₅S⁺=1056.5; found 1056.6.

¹H NMR (400 MHz, Chloroform-d) δ 8.66 (s, 1H), 8.40 (d, J=2.6 Hz, 1H), 8.24 (d, J=2.3 Hz, 1H), 7.68 (dd, J=8.9, 2.6 Hz, 1H), 7.53 (d, J=8.4 Hz, 2H), 7.42-7.34 (m, 5H), 7.27 (d, J=5.5 Hz, 3H), 7.24-7.16 (m, 4H), 6.73 (d, J=9.0 Hz, 1H), 6.35 (d, J=8.7 Hz, 1H), 6.26 (d, J=8.8 Hz, 1H), 5.38 (d, J=8.0 Hz, 1H), 5.14-5.02 (m, 1H), 4.71 (t, J=7.8 Hz, 1H), 4.59 (m, J=8.7 Hz, 2H), 4.53-4.49 (m, 1H), 4.46 (t, J=5.9 Hz, 1H), 4.37 (m, J=6.0 Hz, 4H), 4.04 (d, J=11.3 Hz, 1H), 3.66-3.58 (dd, 1H), 3.54 (s, 1H), 2.99-2.86 (m, 2H), 2.51 (s, 3H), 2.49-2.36 (m, 2H), 2.06 (m, J=15.1, 9.3 Hz, 3H), 2.02-1.87 (m, 4H), 1.79 (m, J=4.0 Hz, 2H), 1.49 (d, 3H), 1.41 (m, J=3.1 Hz, 2H), 1.39-1.32 (m, 2H), 1.25 (m, 2H), 1.05 (s, 9H).

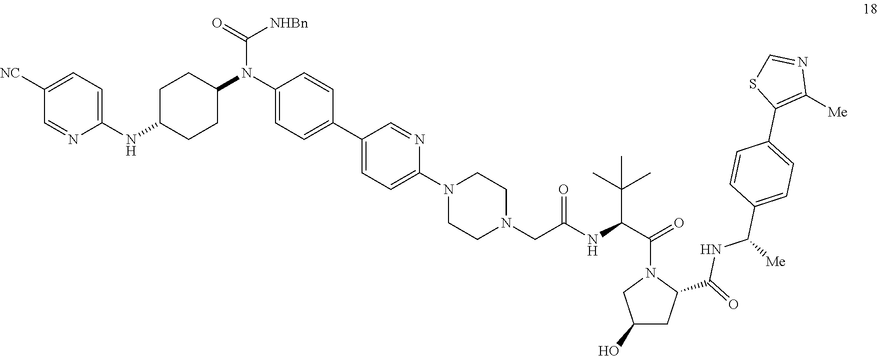

Following the preparation method for Formula 1, use intermediate h0-0 instead of intermediate e0-1.

LC-MS (ESI) m/z (M+H)⁺ calculated for C₆₀H₇₁N₁₂O₅S⁺=1071.5; found 1071.6.

¹H NMR (400 MHz, Chloroform-d) δ 8.64 (s, 1H), 8.43 (d, J=2.5 Hz, 1H), 8.25 (dd, J=2.3, 0.7 Hz, 1H), 7.93 (d, J=8.5 Hz, 1H), 7.71 (dd, J=8.8, 2.6 Hz, 1H), 7.54 (d, J=8.4 Hz, 2H), 7.49 (d, J=7.7 Hz, 1H), 7.44-7.34 (m, 4H), 7.31-7.14 (m, 7H), 6.75 (d, J=8.8 Hz, 1H), 6.27 (dd, J=8.9, 0.8 Hz, 1H), 5.23 (d, J=8.0 Hz, 1H), 5.13-5.02 (m, 1H), 4.76 (t, J=7.9 Hz, 1H), 4.67-4.56 (m, 1H), 4.51 (d, J=8.6 Hz, 2H), 4.48-4.42 (m, 1H), 4.38 (d, J=5.8 Hz, 2H), 4.18-4.06 (m, 2H), 3.73-3.59 (m, 7H), 3.11 (d, J=3.5 Hz, 2H), 2.70 (t, J=5.4 Hz, 4H), 2.50 (s, 3H), 2.10 (d, J=8.7 Hz, 2H), 1.99 (d, J=11.2 Hz, 2H), 1.47 (d, J=6.9 Hz, 3H), 1.42-1.29 (m, 4H), 1.08 (s, 9H).

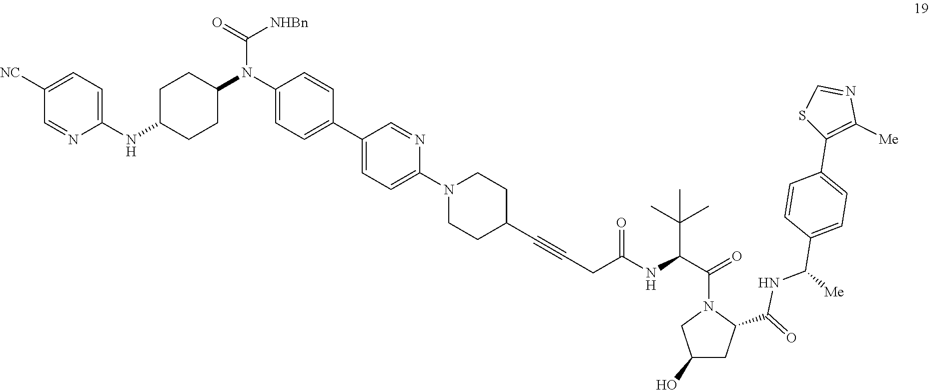

Following the preparation method for Formula 1, use intermediate h0-b instead of intermediate e0-1.

Wherein, the preparation method for intermediate h0-b is by reference to Formula h0-1, using intermediate 1h-b instead of intermediate 1h-1; and the preparation method for intermediate 1h-b is by reference to Formula 1d, using tert-butyl 4-(piperidin-4-yl)but-3-ynoate instead of tert-butyl piperazine-1-carboxylate.

LC-MS (ESI) m/z (M+H)⁺ calculated for C₆₃H₇₂N₁₁O₅S⁺=1094.5; found 1094.6.

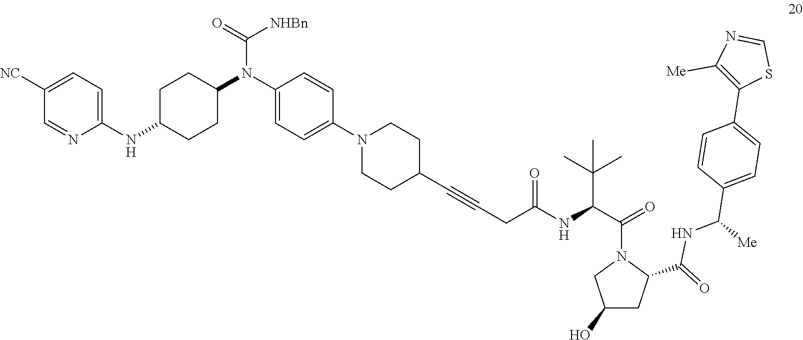

Following the preparation method for Formula 1, use intermediate g0-a instead of intermediate e0-1.

Wherein, the preparation method for intermediate g0-a is by reference to Formula 1c, using tert-butyl 4-(piperidin-4-yl)but-3-ynoate instead of tert-butyl piperazine-1-carboxylate.

LC-MS (ESI) m/z (M+H)⁺ calculated for C₅₈H₆₉N₁₀O₅S⁺=1017.5; found 1017.5.

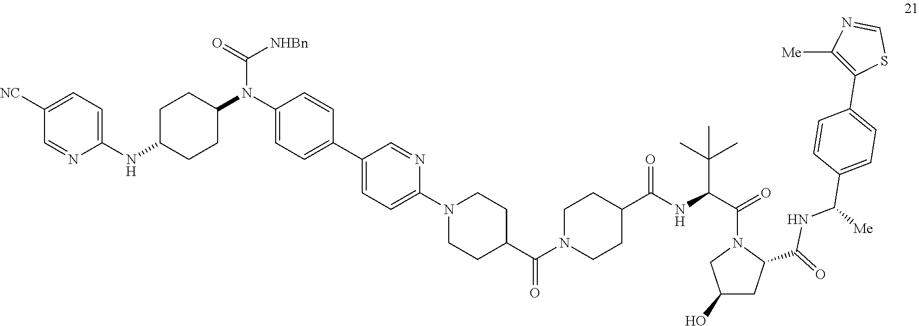

Following the preparation method for Formula 1, use intermediate h0-c instead of intermediate e0-1.

Wherein, the preparation method for intermediate h0-c is by reference to Formula h0-1, using intermediate 1h-c instead of intermediate 1h-1; and the preparation method for intermediate 1h-c is by reference to Formula 1d, using tert-butyl 1-(piperidine-4-carbonyl)piperidine-4-carboxylate instead of tert-butyl piperazine-1-carboxylate.

LC-MS (ESI) m/z (M+H)⁺ calculated for C₆₆H₇₉N₁₂O₆S⁺=1167.6; found 1167.7.

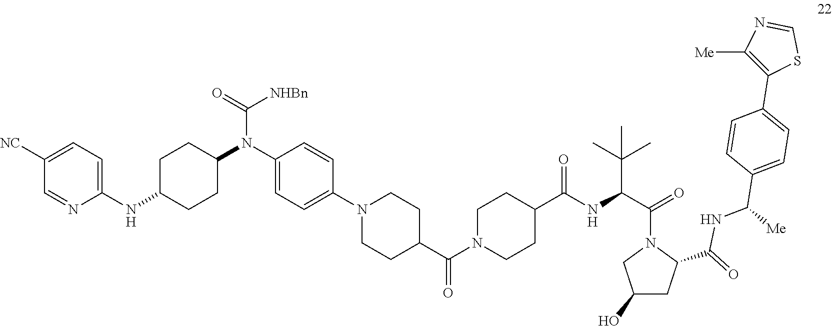

Following the preparation method for Formula 1, use intermediate g0-b instead of intermediate e0-1.

Wherein, the preparation method for intermediate g0-b is by reference to Formula 1c, using tert-butyl 1-(piperidine-4-carbonyl)piperidine-4-carboxylate instead of tert-butyl piperazine-1-carboxylate.

LC-MS (ESI) m/z (M+H)⁺ calculated for C₆₁H₇₆N₁₁O₆S⁺=1090.6; found 1090.7.

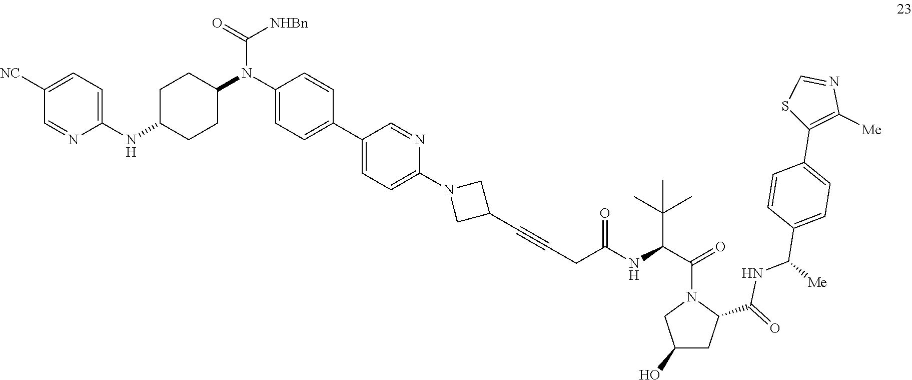

Following the preparation method for Formula 1, use intermediate h0-d instead of intermediate e0-1.

Wherein, the preparation method for intermediate h0-d is by reference to Formula h0-1, using intermediate 1h-d instead of intermediate 1h-1; and the preparation method for intermediate 1h-d is by reference to Formula 1d, using tert-butyl 4-(azetidin-3-yl)but-3-ynoate instead of tert-butyl piperazine-1-carboxylate.

LC-MS (ESI) m/z (M+H)⁺ calculated for C₆₁H₆₈N₁₁O₅S⁺=1066.5; found 1066.5.

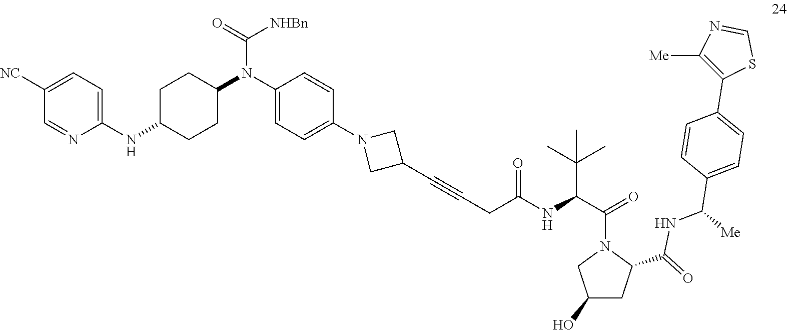

Following the preparation method for Formula 1, use intermediate g0-c instead of intermediate e0-1.

Wherein, the preparation method for intermediate g0-c is by reference to Formula 1c, using tert-butyl 4-(azetidin-3-yl)but-3-ynoate instead of tert-butyl piperazine-1-carboxylate.

LC-MS (ESI) m/z (M+H)⁺ calculated for C₅₆H₆₅N₁₀O₅S⁺=989.5; found 989.5.

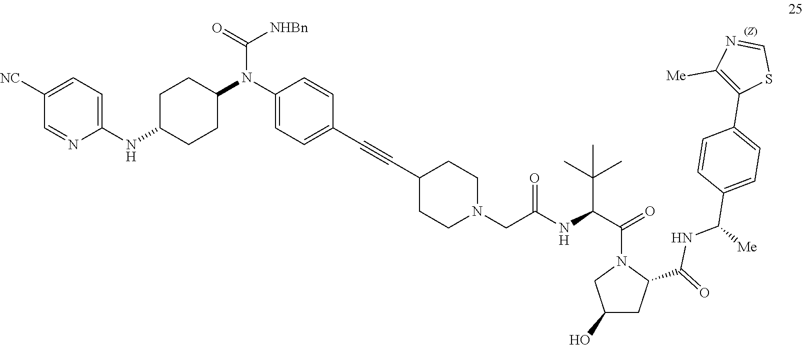

Following the preparation method for Formula 1, use intermediate g0-d instead of intermediate e0-1.

Wherein, the preparation method for intermediate g0-d is by subjecting intermediate 1a to a Sonogashira coupling reaction with tert-butyl 2-(4-ethynylpiperidin-1-yl)acetate to obtain it. The specific conditions can be found in the following reference: Chemistry Letters, 2005, vol. 34, issue 9, 1294-1295.

LC-MS (ESI) m/z (M+H)⁺ calculated for C₅₈H₆₉N₁₀O₅S⁺=1017.5; found 1017.5.

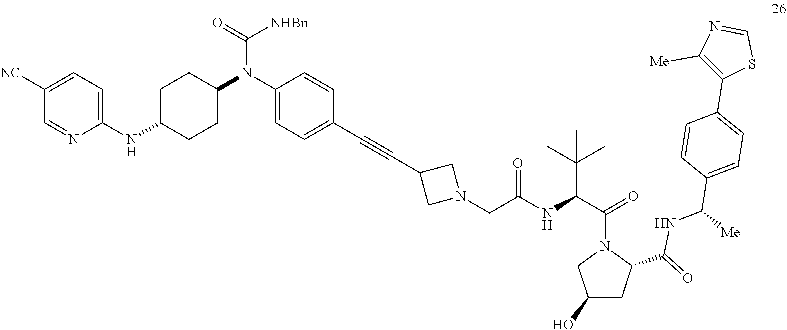

Following the preparation method for Formula 1, use intermediate g0-e instead of intermediate e0-1.

Wherein, the preparation method for intermediate g0-e is by subjecting intermediate 1a to a Sonogashira coupling reaction with tert-butyl 2-(4-ethynylpiperidin-1-yl)acetate to obtain it. The specific conditions can be found in the following reference: Chemistry Letters, 2005, vol. 34, issue 9, 1294-1295.

LC-MS (ESI) m/z (M+H)⁺ calculated for C₅₆H₆₅N₁₀O₅S⁺=989.5; found 989.5.

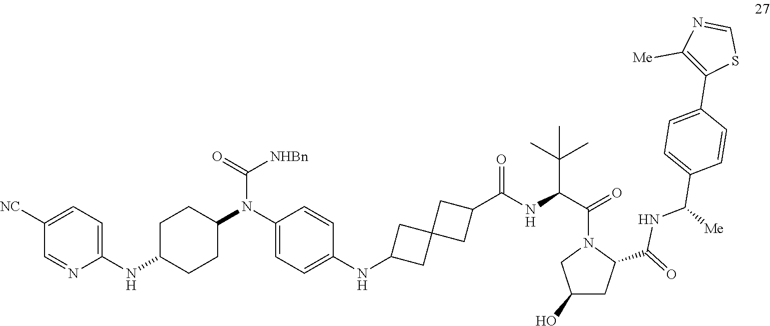

Following the preparation method for Formula 1, use intermediate g0-f instead of intermediate e0-1.

Wherein, the preparation method for intermediate g0-f is by reference to Formula 1c, using tert-butyl 2-aminospiro[3.3]heptane-6-carboxylate instead of tert-butyl piperazine-1-carboxylate.

LC-MS (ESI) m/z (M+H)⁺ calculated for C₅₇H₆₉N₁₀O₅S⁺=1005.5; found 1005.6.

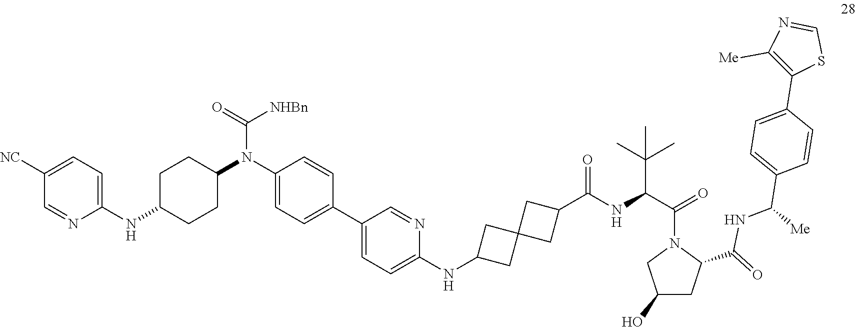

Following the preparation method for Formula 1, use intermediate h0-e instead of intermediate e0-1.

Wherein, the preparation method for intermediate h0-e is by reference to Formula h0-1, using intermediate 1h-e instead of intermediate 1h-1; and the preparation method for intermediate 1h-e is by reference to Formula 1d, using tert-butyl 2-aminospiro[3.3]heptane-6-carboxylate instead of tert-butyl piperazine-1-carboxylate.

LC-MS (ESI) m/z (M+H)⁺ calculated for C₆₂H₇₂N₁₁O₅S⁺=1082.5; found 1082.6.

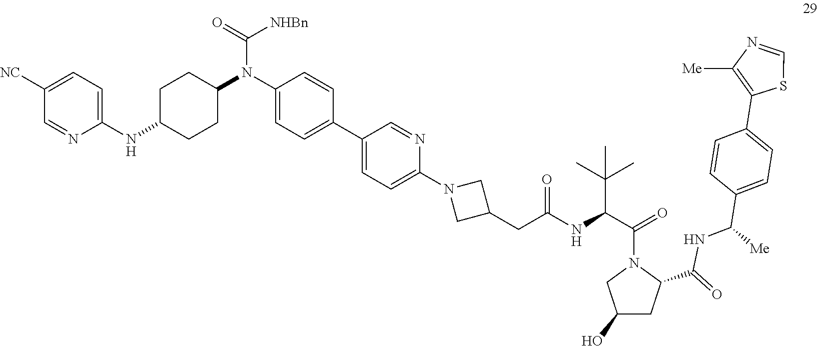

Following the preparation method for Formula 1, use intermediate h0-f instead of intermediate e0-1.

Wherein, the preparation method for intermediate h0-f is by reference to Formula h0-1, using intermediate 1h-f instead of intermediate 1h-1; and the preparation method for intermediate 1h-f is by reference to Formula 1d, using tert-butyl 2-(azetidin-3-yl)acetate instead of tert-butyl piperazine-1-carboxylate.

LC-MS (ESI) m/z (M+H)⁺ calculated for C₅₉H₆₈N₁₁O₅S⁺=1042.5; found 1042.5.

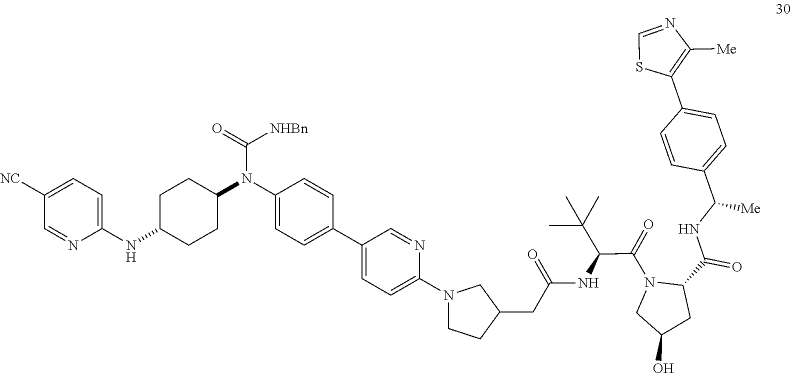

Following the preparation method for Formula 1, use intermediate h0-g instead of intermediate e0-1.

Wherein, the preparation method for intermediate h0-g is by reference to Formula h0-1, using intermediate 1h-g instead of intermediate 1h-1; and the preparation method for intermediate 1h-g is by reference to Formula 1d, using tert-butyl pyrrolidin-3-ylacetate instead of tert-butyl piperazine-1-carboxylate.

LC-MS (ESI) m/z (M+H)⁺ calculated for C₆₀H₇₀N₁₁O₅S⁺=1056.5; found 1056.6.

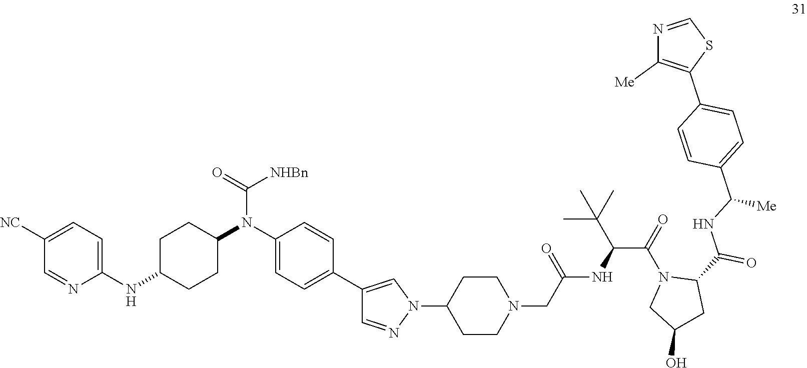

Following the preparation method for Formula 1, use intermediate h0-h instead of intermediate e0-1.

Wherein, the preparation method for intermediate h0-h is by reference to Formula h0-1, using intermediate 1h-h instead of intermediate 1h-1; and the preparation method for intermediate 1h-h is by reference to Formula 1h-1, with the starting materials being 4-(4-bromo-1H-pyrazol-1-yl)piperidine and tert-butyl bromoacetate.

LC-MS (ESI) m/z (M+H)⁺ calculated for C₅₉H₇₁N₁₂O₅S⁺=1059.5; found 1059.6.

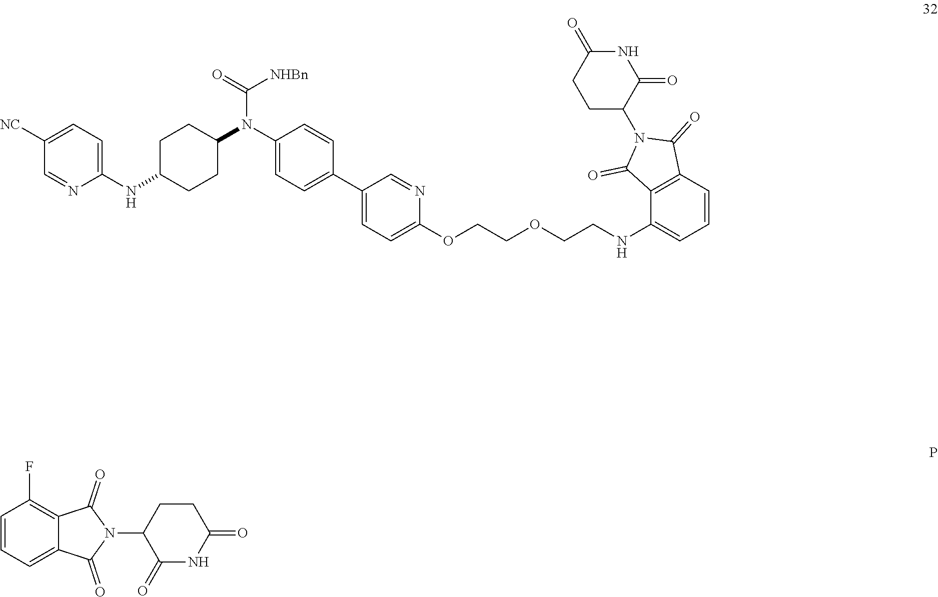

In a 10 mL round-bottom flask, 70 mg of intermediate a0-1 and 35 mg of intermediate P are added and dissolved in 4 ml of anhydrous DMSO. While stirring at room temperature, slowly add 95 mg of DIPEA dropwise to the system. Then, heat to 100 °C and react for 8h.

After TLC monitoring shows the reaction is complete, wash with water and extract with ethyl acetate. Concentrate the organic phase under reduced pressure and rotary dry. Purify by silica gel column chromatography using a 20:1 (v/v) mixture of dichloromethane:methanol as the eluent to obtain the compound as shown in Formula 32, with a yield of 36%.

The preparation of 2-(2,6-dioxopiperidin-3-yl)-4-fluoroisoindoline-1,3-dione (Formula P) can be found in the following reference: Organic Letters 22, 10, 3838-3841 (2019).

LC-MS (ESI) m/z (M+H)⁺ calculated for C₄₈H₄₈N₉O₇S⁺=862.36; found 862.4.

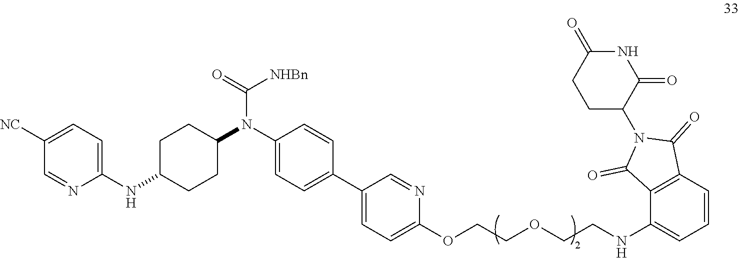

Following the preparation method for Formula 32, use intermediate a0-2 instead of intermediate a0-1.

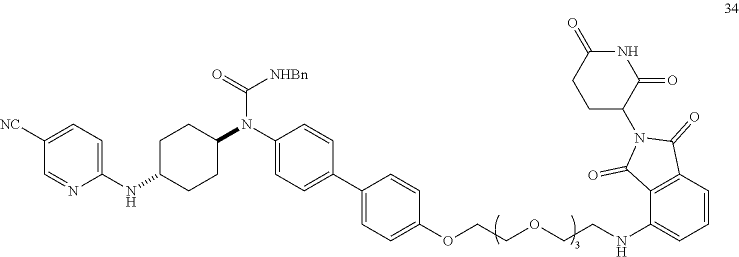

LC-MS (ESI) m/z (M+H)⁺ calculated for C₅₂H₅₆N₉O₉⁺=950.4; found 950.5.

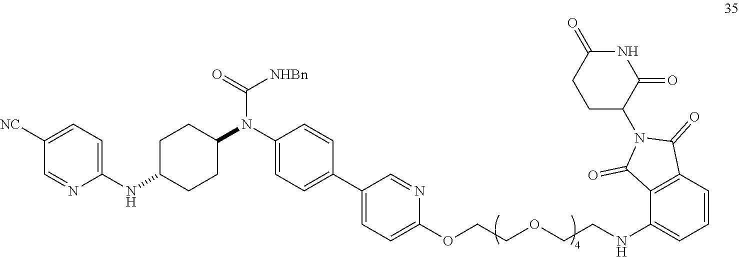

Following the preparation method for Formula 32, use intermediate a0-4 instead of intermediate a0-1.

LC-MS (ESI) m/z (M+H)⁺ calculated for C₅₄H₆₀N₉O₁₀⁺=994.4; found 994.5.

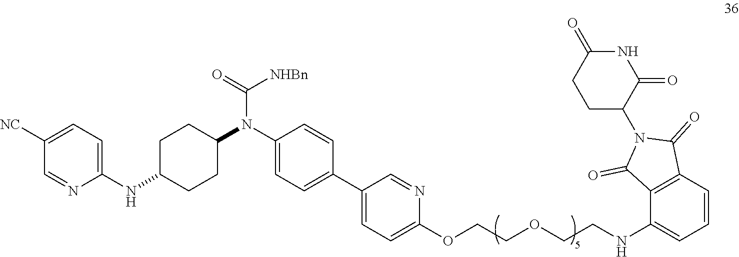

Following the preparation method for Formula 32, use intermediate a0-5 instead of intermediate a0-1.

LC-MS (ESI) m/z (M+H)⁺ calculated for C₅₆H₆₄N₉O₁₁⁺=1038.5; found 1038.6.

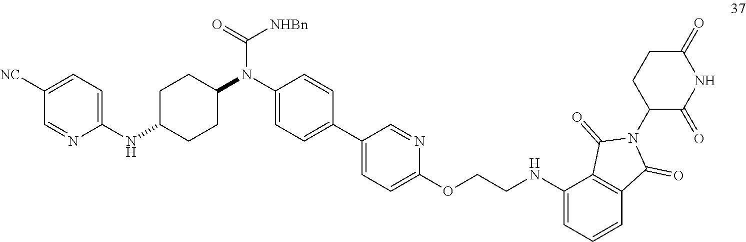

Following the preparation method for Formula 32, use intermediate a0-0 instead of intermediate a0-1.

LC-MS (ESI) m/z (M+H)⁺ calculated for C₄₆H₄₄N₉O₆⁺=818.3; found 818.4.

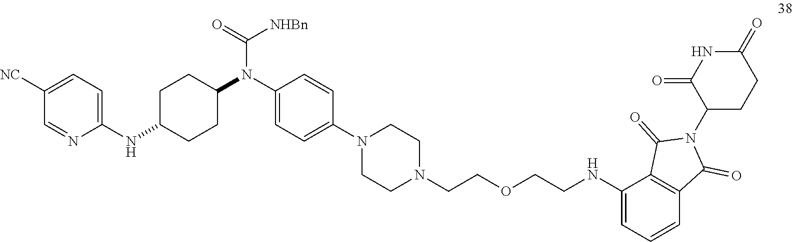

Following the preparation method for Formula 32, use intermediate c0-1 instead of intermediate a0-1.

LC-MS (ESI) m/z (M+H)⁺ calculated for C₄₇H₅₃N₁₀O₆⁺=853.4; found 853.4.

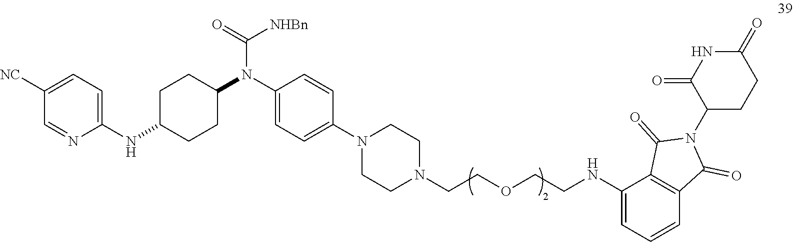

Following the preparation method for Formula 32, use intermediate c0-2 instead of intermediate a0-1.

LC-MS (ESI) m/z (M+H)⁺ calculated for C₄₉H₅₇N₁₀O₇⁺=897.4; found 897.5.

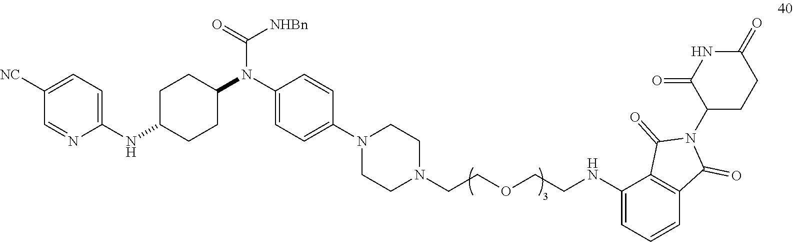

Following the preparation method for Formula 32, use intermediate c0-3 instead of intermediate a0-1.

LC-MS (ESI) m/z (M+H)⁺ calculated for C₅₁H₆₅N₁₀O₈⁺=941.5; found 941.6.

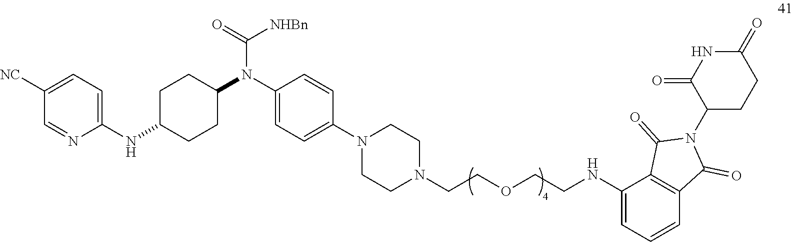

Following the preparation method for Formula 32, use intermediate c0-4 instead of intermediate a0-1.

LC-MS (ESI) m/z (M+H)⁺ calculated for C₅₃H₆₅N₁₀O₉⁺=985.5; found 985.6.

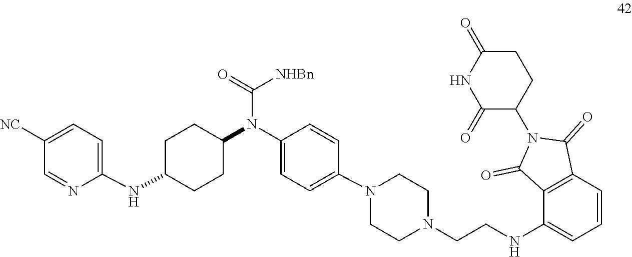

Following the preparation method for Formula 32, use intermediate c0-0 instead of intermediate a0-1.

LC-MS (ESI) m/z (M+H)⁺ calculated for C₄₅H₄₉N₁₀O₅⁺=809.4; found 809.4.

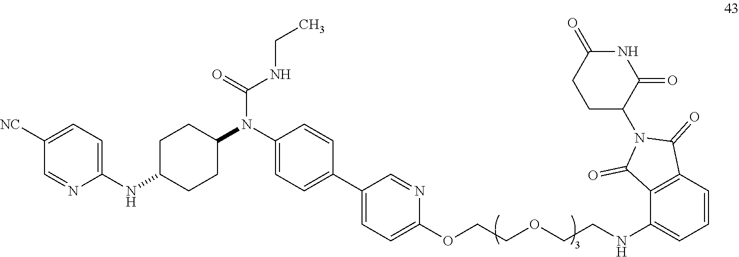

Following the preparation method for Formula 32, use intermediate a0-3-Et instead of intermediate a0-1.

Wherein, the preparation method for intermediate a0-3-Et is by reference to Formula a0-3, using intermediate 5aa-Et instead of intermediate 5aa; wherein, the preparation method for intermediate 5aa-Et is by reference to Formula 5aa, using ethyl isocyanate instead of intermediate 4aa. LC-MS (ESI) m/z (M+H)⁺ calculated for C₄₇H₅₄N₉O₉⁺=888.4; found 888.4.

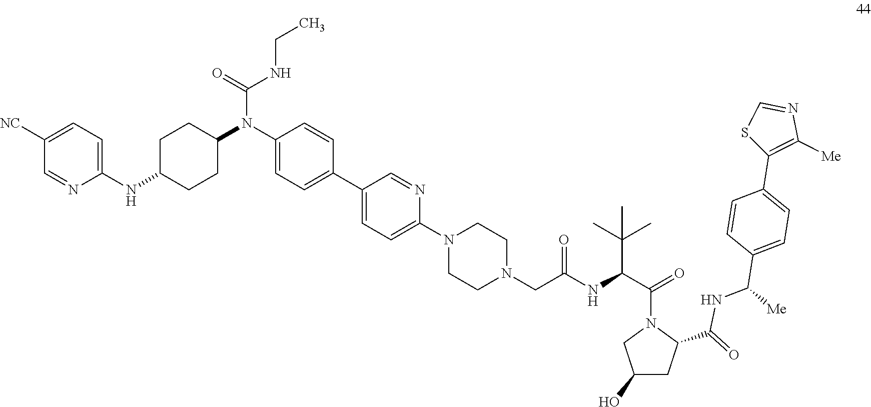

Following the preparation method for Formula 32, use intermediate h0-0-Et instead of intermediate e0-1.

Wherein, the preparation method for intermediate h0-0-Et is by reference to Formula h0-0, using intermediate 5aa-Et instead of intermediate 5aa; wherein, the preparation method for intermediate 5aa-Et is by reference to Formula 5aa, using ethyl isocyanate instead of intermediate 4aa.

LC-MS (ESI) m/z (M+H)⁺ calculated for C₅₅H₆₉N₁₂O₅S+=1009.5; found 1009.5.

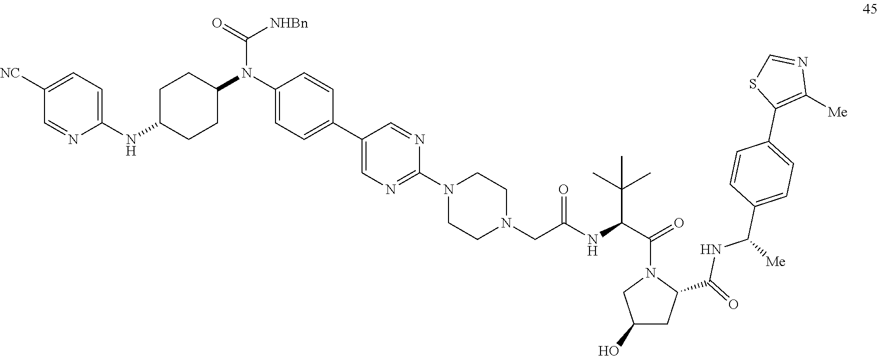

Following the preparation method for Formula 32, use intermediate h0-0-N instead of intermediate e0-1.

Wherein, the preparation method for intermediate h0-0-N is by reference to Formula h0-0, using intermediate 1d-N instead of intermediate 1d; wherein, the preparation method for intermediate 1d-N is by reference to Formula 1d, using 5-bromo-2-fluoropyrimidine instead of 2-fluoro-5-bromopyridine.

LC-MS (ESI) m/z (M+H)⁺ calculated for C₅₉H₇₀N₁₃O₅S+=1072.5; found 1072.6.

¹H NMR (400 MHz, Chloroform-d) δ 8.66 (s, 1H), 8.55 (s, 2H), 8.27 (d, J=2.2 Hz, 1H), 7.92 (d, J=8.4 Hz, 1H), 7.54-7.35 (m, 8H), 7.32-7.17 (m, 6H), 6.27 (d, J=8.7 Hz, 1H), 5.06 (dt, J=14.4, 7.5 Hz, 2H), 4.77 (t, J=7.8 Hz, 1H), 4.62 (s, 1H), 4.53 (s, 1H), 4.49 (d, J=8.4 Hz, 1H), 4.38 (s, 3H), 4.17 (d, J=11.5 Hz, 1H), 3.94 (s, 4H), 3.61 (dd, J=11.4, 3.7 Hz, 1H), 3.11 (d, J=2.7 Hz, 2H), 2.66 (t, J=5.1 Hz, 4H), 2.58 (ddd, J=13.5, 7.5, 4.7 Hz, 1H), 2.52 (s, 3H), 2.10 (d, J=12.3 Hz, 2H), 2.00 (d, J=11.7 Hz, 2H), 1.78 (s, 2H), 1.48 (d, J=6.9 Hz, 3H), 1.45-1.41 (m, 2H), 1.38-1.27 (m, 4H), 1.09 (s, 9H).

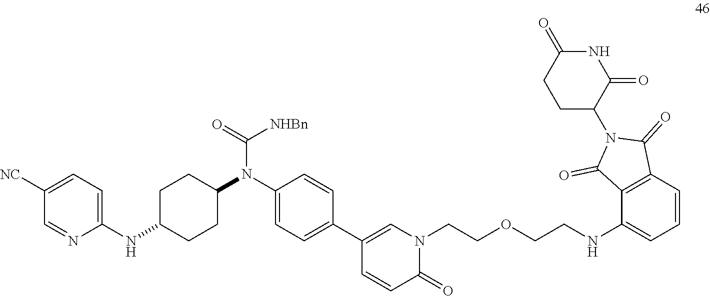

Following the preparation method for Formula 32, use intermediate a0-1-N instead of intermediate a0-1.

Wherein, the preparation method for intermediate a0-1-N is by reference to Formula a0-1, using intermediate 5a-1-N instead of intermediate 5a-1; wherein, the preparation method for intermediate 5a-1-N is by subjecting 2c-1 to a nucleophilic reaction with 5-bromo-2-hydroxypyridine to obtain it.

LC-MS (ESI) m/z (M+H)⁺ calculated for C₄₈H₄₈N₉O₇⁺=862.4; found 862.4.

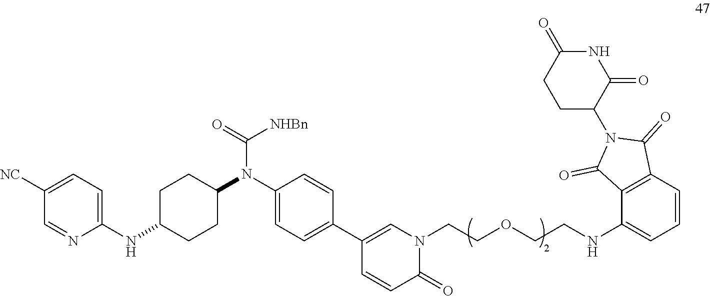

Following the preparation method for Formula 32, use intermediate a0-2-N instead of intermediate a0-1.

Wherein, the preparation method for intermediate a0-2-N is by reference to Formula a0-1, using intermediate 5a-2-N instead of intermediate 5a-1; wherein, the preparation method for intermediate 5a-2-N is by subjecting 2c-2 to a nucleophilic reaction with 5-bromo-2-hydroxypyridine to obtain it.

LC-MS (ESI) m/z (M+H)⁺ calculated for C₅₀H₅₂N₉O₈⁺=906.3; found 906.4.

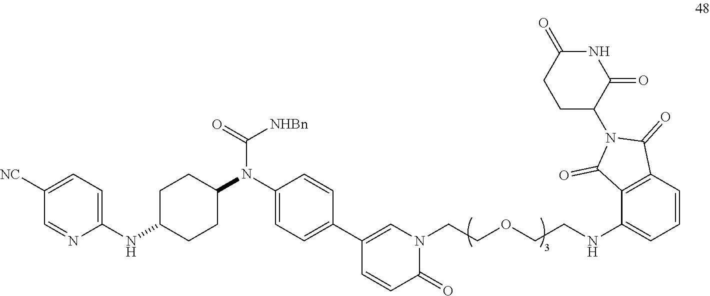

Following the preparation method for Formula 32, use intermediate a0-3-N instead of intermediate a0-1.

Wherein, the preparation method for intermediate a0-3-N is by reference to Formula a0-1, using intermediate 5a-3-N instead of intermediate 5a-1; wherein, the preparation method for intermediate 5a-3-N is by subjecting 2c-3 to a nucleophilic reaction with 5-bromo-2-hydroxypyridine to obtain it.

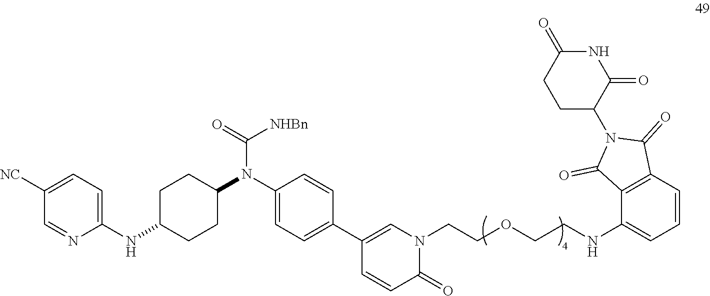

Following the preparation method for Formula 32, use intermediate a0-4-N instead of intermediate a0-1.

Wherein, the preparation method for intermediate a0-4-N is by reference to Formula a0-1, using intermediate 5a-4-N instead of intermediate 5a-1; wherein, the preparation method for intermediate 5a-4-N is by subjecting 2c-4 to a nucleophilic reaction with 5-bromo-2-hydroxypyridine to obtain it.

LC-MS (ESI) m/z (M+H)⁺ calculated for C₅₄H₆₀N₉O₁₀⁺=994.4; found 994.5.

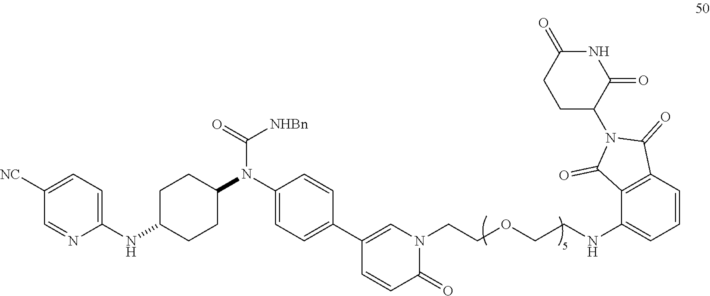

Following the preparation method or Formula 32, use intermediate a0-4-N instead of intermediate a0-1.

Wherein, the preparation method for intermediate a0-4-N is by reference to Formula a0-1, using intermediate 5a-4-N instead of intermediate 5a-1; wherein, the preparation method for intermediate 5a-4-N is by subjecting 2c-4 to a nucleophilic reaction with 5-bromo-2-hydroxypyridine to obtain it.

LC-MS (ESI) m/z (M+H)⁺ calculated for C₅₆H₆₄N₉O₁₁⁺*=1038.4; found 1038.5.

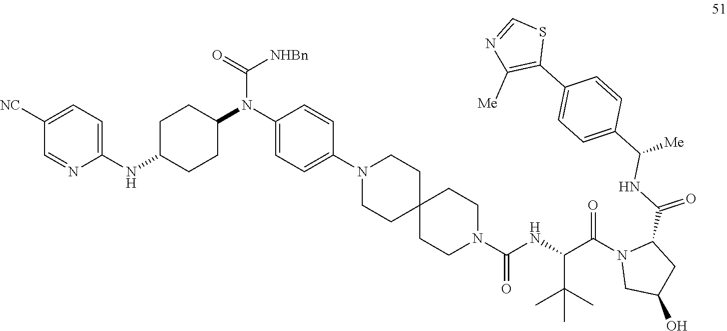

In a 10 mL round-bottom flask, add 18 mg of intermediate V and 6 mg of CDI, and dissolve in 2 ml of anhydrous dichloromethane. Stir at room temperature for 1 hour. Then, add 18 mg of intermediate g0-h and react for 18h.

After TLC monitoring shows the reaction is complete, wash with water and extract with ethyl acetate. Concentrate the organic phase under reduced pressure and rotary dry. Purify by silica gel column chromatography using a 20:1 (v/v) mixture of dichloromethane:methanol as the eluent to obtain the compound as shown in Formula 51, with a yield of 47%.

Wherein, the preparation method for intermediate g0-h is by reference to Formula 1c, using tert-butyl 1,9-diazaspiro[5.5]undecane-3-carboxylate instead of tert-butyl piperazine-1-carboxylate.

LC-MS (ESI) m/z (M+H)⁺ calculated for C₅₉H₇₄N₁₁O₅S⁺=1048.5; found 1048.6.

¹H NMR (400 MHz, Chloroform-d) δ 8.67 (s, 1H), 7.63 (d, J=7.9 Hz, 1H), 7.46 (dd, J=8.8, 2.3 Hz, 1H), 7.43-7.36 (m, 4H), 7.29-7.15 (m, 4H), 7.04-6.98 (m, 2H), 6.92-6.86 (m, 2H), 6.26 (dd, J=8.8, 0.8 Hz, 1H), 5.08 (p, J=7.1 Hz, 1H), 4.99 (d, J=8.9 Hz, 1H), 4.92 (d, J=8.0 Hz, 1H), 4.80 (t, J=8.2 Hz, 1H), 4.60-4.50 (m, 1H), 4.46 (s, 1H), 4.42-4.30 (m, 5H), 3.68 (s, 1H), 3.54-3.47 (m, 2H), 3.43-3.30 (m, 4H), 3.20 (t, J=5.7 Hz, 4H), 2.59 (ddd, J=13.1, 8.0, 4.3 Hz, 1H), 2.53 (s, 3H), 2.09 (m, J=13.8, 9.0 Hz, 3H), 1.94 (d, J=12.0 Hz, 2H), 1.53 (t, J=5.8 Hz, 4H), 1.46 (d, J=7.0 Hz, 2H), 1.41-1.28 (m, 4H), 1.07 (s, 9H).

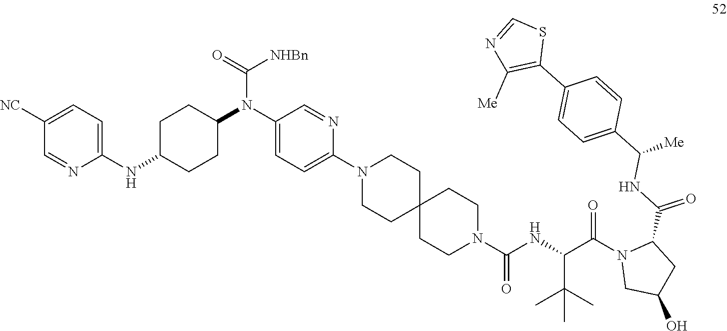

Following the preparation method for Formula 51, use intermediate h0-i instead of intermediate g0-h.

Wherein, the preparation method for intermediate h0-i is by reference to Formula h0-1, using intermediate 1h-i instead of intermediate 1h-1; and the preparation method for intermediate 1h-i is by reference to Formula 1d, using tert-butyl 3,9-diazaspiro[5.5]undecane-3-carboxylate instead of tert-butyl piperazine-1-carboxylate.

LC-MS (ESI) m/z (M+H)⁺ calculated for C₆₄H₇₇N₁₂O₅S⁺=1125.6; found 1125.6.

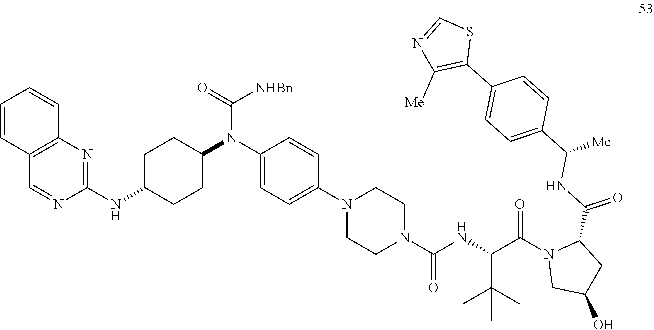

Following the preparation method for Formula 51, use intermediate g0-j instead of intermediate g0-h.

Wherein, the preparation method for intermediate g0-j is by reference to Formula 1c, using tert-butyl piperazine-1-carboxylate instead of tert-butyl piperazine-1-carboxylate.

LC-MS (ESI) m/z (M+H)⁺ calculated for C₅₄H₆₆N₁₁O₅*=980.5; found 980.5.

¹H NMR (400 MHz, Chloroform-d) δ 8.66 (s, 1H), 8.43 (d, J=2.5 Hz, 1H), 8.26 (dd, J=2.3, 0.7 Hz, 1H), 7.72 (dd, J=8.8, 2.5 Hz, 1H), 7.60 (d, J=7.9 Hz, 1H), 7.57-7.52 (m, 2H), 7.43-7.36 (m, 5H), 7.31-7.26 (m, 2H), 7.24-7.17 (m, 5H), 6.70 (d, J=8.8 Hz, 1H), 6.26 (dd, J=8.9, 0.8 Hz, 1H), 5.10 (dt, J=14.5, 7.3 Hz, 2H), 4.78 (t, J=8.1 Hz, 1H), 4.62 (td, J=11.8, 6.0 Hz, 1H), 4.49 (s, 1H), 4.45-4.35 (m, 4H), 4.25 (d, J=11.6 Hz, 1H), 3.71-3.61 (m, 4H), 3.60-3.50 (m, 4H), 2.57 (m, J=8.3, 4.9 Hz, 1H), 2.52 (s, 3H), 2.15-2.07 (d, 2H), 2.03-1.96 (d, 2H), 1.89 (m, 2H), 1.47 (d, J=6.9 Hz, 3H), 1.43-1.22 (m, 6H), 1.08 (s, 9H).

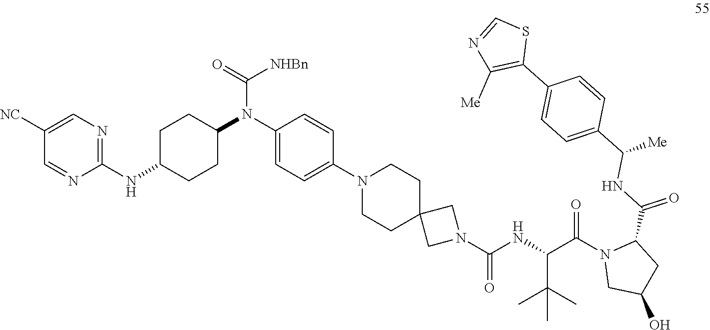

Following the preparation method for Formula 51, use intermediate g0-k instead of intermediate g0-h.

Wherein, the preparation method for intermediate g0-k is by reference to Formula 1c, using tert-butyl 2,7-diazaspiro[3.5]nonane-7-carboxylate instead of tert-butyl piperazine-1-carboxylate.

LC-MS (ESI) m/z (M+H)⁺ calculated for C₅₇H₇₀N₁₁O₅S⁺=1020.5; found 1020.6.

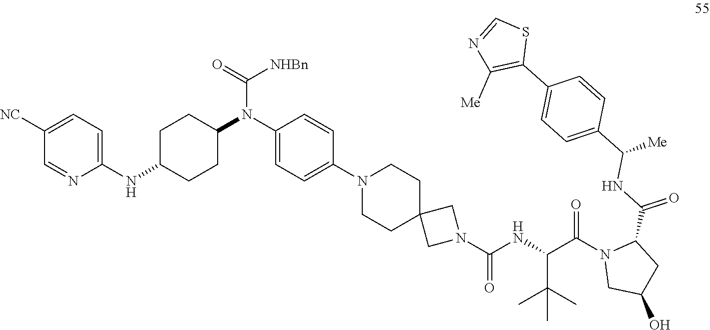

Following the preparation method for Formula 51, use intermediate g0-1 instead of intermediate g0-h.

Wherein, the preparation method for intermediate g0-1 is by reference to Formula 1c, using tert-butyl 2,7-diazaspiro[3.5]nonane-2-carboxylate instead of tert-butyl piperazine-1-carboxylate.

LC-MS (ESI) m/z (M+H)⁺ calculated for C₅₇H₇₀N₁₁O₅S⁺=1020.5; found 1020.6.

¹H NMR (400 MHz, Chloroform-d) δ 8.67 (s, 1H), 8.27 (d, J=2.2 Hz, 1H), 7.56 (d, J=7.9 Hz, 1H), 7.45 (dd, J=8.9, 2.3 Hz, 2H), 7.38 (dd, J=8.7, 6.3 Hz, 4H), 7.27 (s, 1H), 7.01 (d, J=8.5 Hz, 2H), 6.89 (d, J=8.8 Hz, 2H), 6.26 (d, J=8.8 Hz, 1H), 5.08 (p, J=7.1 Hz, 1H), 4.97 (d, J=7.9 Hz, 1H), 4.77 (t, J=8.0 Hz, 1H), 4.70 (d, J=9.3 Hz, 1H), 4.58-4.50 (m, 1H), 4.48 (d, J=4.8 Hz, 1H), 4.37 (dt, J=14.1, 4.0 Hz, 4H), 4.23 (d, J=11.6 Hz, 1H), 3.74-3.64 (m, 5H), 3.57-3.46 (m, 2H), 3.16 (t, J=5.4 Hz, 4H), 2.53 (s, 3H), 2.12-1.83 (m, 10H), 1.47 (d, J=6.9 Hz, 3H), 1.27 (d, J=12.3 Hz, 12H), 1.05 (s, 9H).

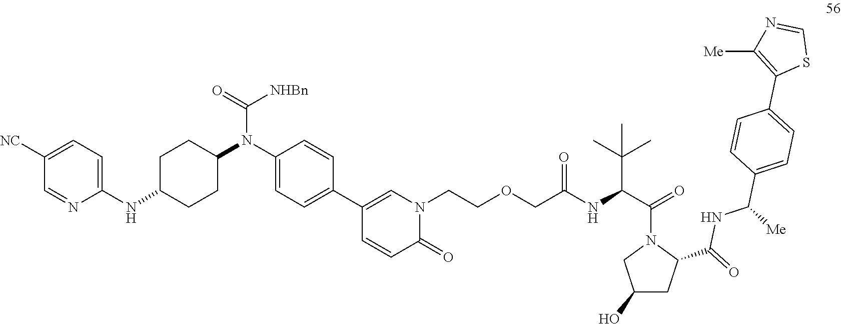

Following the preparation method for Formula 1, use intermediate e0-1-N instead of intermediate a0-1.

Wherein, the preparation method for intermediate e0-1-N is by reference to Formula e0-1, using intermediate 1e-1-N instead of intermediate 1e-1; and the preparation method for intermediate 1e-1-N is by subjecting 1g-1 to a nucleophilic reaction with 5-bromo-2-hydroxypyridine to obtain it.

LC-MS (ESI) m/z (M+H)⁺ calculated for C₅₈H₆₇N₁₀O₇S⁺=1047.5; found 1047.5.

¹H NMR (400 MHz, Chloroform-d) δ 8.98 (s, 1H), 8.43 (d, J=7.8 Hz, 1H), 8.28 (d, J=2.4 Hz, 1H), 8.18 (d, J=2.9 Hz, 1H), 7.89 (dd, J=9.5, 2.6 Hz, 1H), 7.69 (d, J=8.5 Hz, 2H), 7.59 (dd, J=8.9, 2.5 Hz, 1H), 7.53-7.46 (m, 1H), 7.45-7.40 (m, 2H), 7.38-7.31 (m, 3H), 7.29-7.21 (m, 4H), 7.20-7.16 (m, 3H), 6.52 (d, J=9.5 Hz, 1H), 6.46 (d, J=8.9 Hz, 1H), 5.84 (t, J=6.2 Hz, 1H), 5.14 (d, J=3.5 Hz, 1H), 4.95-4.84 (m, 1H), 4.49 (d, J=9.5 Hz, 1H), 4.43 (t, J=8.2 Hz, 1H), 4.36-4.19 (m, 4H), 4.17 (d, J=5.9 Hz, 2H), 4.05-3.94 (m, 2H), 3.88-3.76 (m, 2H), 3.56 (s, 2H), 2.45 (s, 3H), 2.10-2.00 (m, 1H), 1.95-1.73 (m, 5H), 1.35 (d, J=7.0 Hz, 3H), 1.32-1.10 (m, 5H), 0.84 (s, 9H); 13C NMR (100 MHz, DMSO-d6) δ 170.5, 169.0, 168.1, 160.8, 159.3, 156.5, 153.1, 151.5, 147.8, 144.7, 141.3, 139.0, 138.4, 137.3, 136.7, 135.5, 131.6, 131.1, 129.7, 128.9, 128.1, 126.8, 126.3, 126.3, 126.2, 119.7, 119.1, 117.0, 108.9, 94.0, 69.4, 68.8, 68.3, 58.6, 56.5, 55.8, 53.3, 48.6, 48.5, 47.8, 43.5, 37.7, 35.6, 31.3, 30.3, 26.2, 22.5, 16.0.

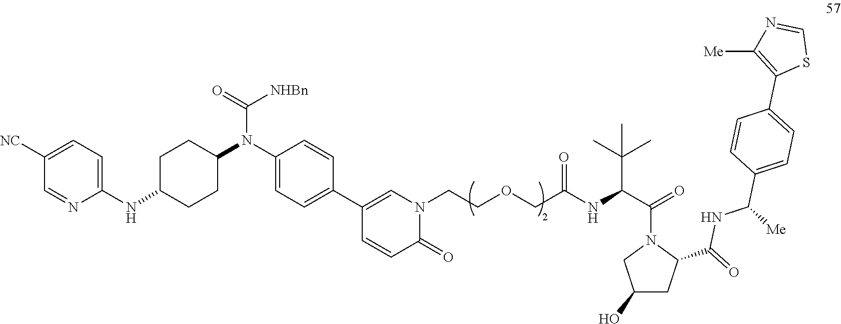

Following the preparation method for Formula 1, use intermediate e0-2-N instead of intermediate a0-1.

Wherein, the preparation method for intermediate e0-2-N is by reference to Formula e0-1, using intermediate 1e-2-N instead of intermediate 1e-2; wherein, the preparation method for intermediate 1e-2-N is by reference to Formula 1e-1-N, using intermediate 1g-2 instead of intermediate 1g-1.

LC-MS (ESI) m/z (M+H)⁺ calculated for C₆₀H₇₁N₁₀O₈S⁺=1091.5; found 1091.6.

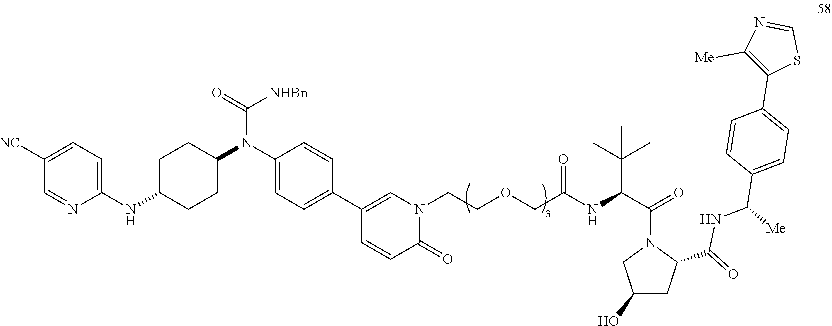

Following the preparation method for Formula 1, use intermediate e0-3-N instead of intermediate a0-1.

Wherein, the preparation method for intermediate e0-3-N is by reference to Formula e0-1, using intermediate 1e-3-N instead of intermediate 1e-1; wherein, the preparation method for intermediate 1e-3-N is by reference to Formula 1e-1-N, using intermediate 1g-3 instead of intermediate 1g-1.

LC-MS (ESI) m/z (M+H)⁺ calculated for C₆₂H₇₅N₁₀O₉S⁺=1135.5; found 1135.6.

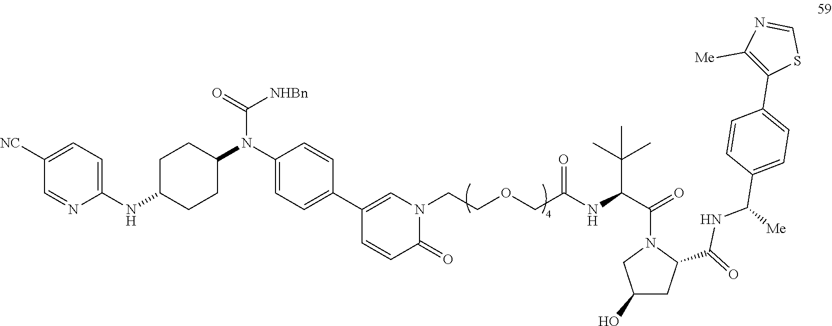

Following the preparation method for Formula 1, use intermediate e0-4-N instead of intermediate a0-1.

Wherein, the preparation method for intermediate e0-4-N is by reference to Formula e0-1, using intermediate 1e-4-N instead of intermediate 1e-1; wherein, the preparation method for intermediate 1e-4-N is by reference to Formula 1e-1-N, using intermediate 1g-4 instead of intermediate 1g-1.

LC-MS (ESI) m/z (M+H)⁺ calculated for C₆₄H₇₉N₁₀O₁₀S⁺=1179.6; found 1179.6.

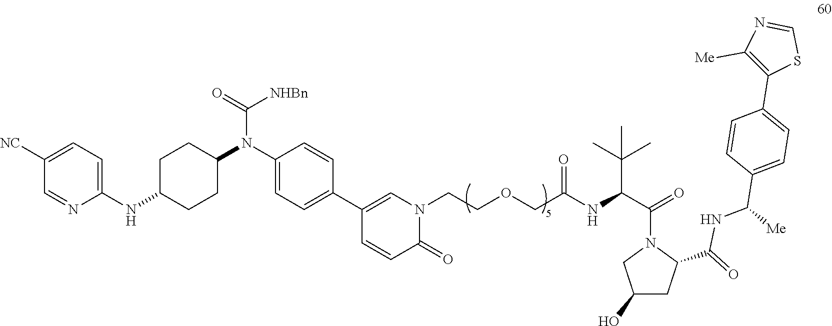

Following the preparation method for Formula 1, use intermediate e0-5-N instead of intermediate a0-1.

Wherein, the preparation method for intermediate e0-5-N is by reference to Formula e0-1, using intermediate 1e-5-N instead of intermediate 1e-1; wherein, the preparation method for intermediate 1e-5-N is by reference to Formula 1e-1-N, using intermediate 1g-5 instead of intermediate 1g-1.

LC-MS (ESI) m/z (M+H)⁺ calculated for C₆₆H₈₃N₁₀O₁₁S⁺=1223.6; found 1223.6.

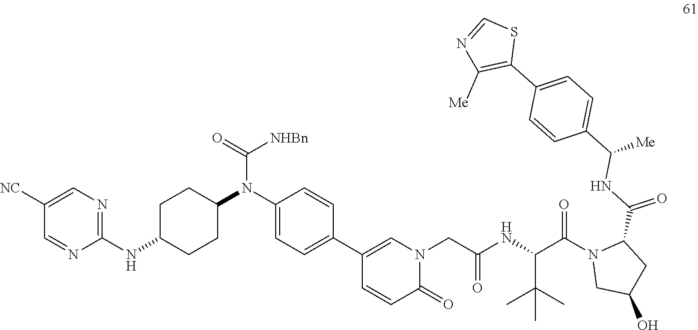

Following the preparation method for Formula 1, use intermediate e0-0-N instead of intermediate a0-1.

Wherein, the preparation method for intermediate e0-0-N is by reference to Formula e0-1, using intermediate 1e-0-N instead of intermediate 1e-1; wherein, the preparation method for intermediate 1e-0-N is by reference to Formula 1e-1-N, using intermediate 1g-0 instead of intermediate 1g-1.

LC-MS (ESI) m/z (M+H)⁺ calculated for C₅₆H₆₃N₁₀O₆S⁺=1003.5; found 1003.5.

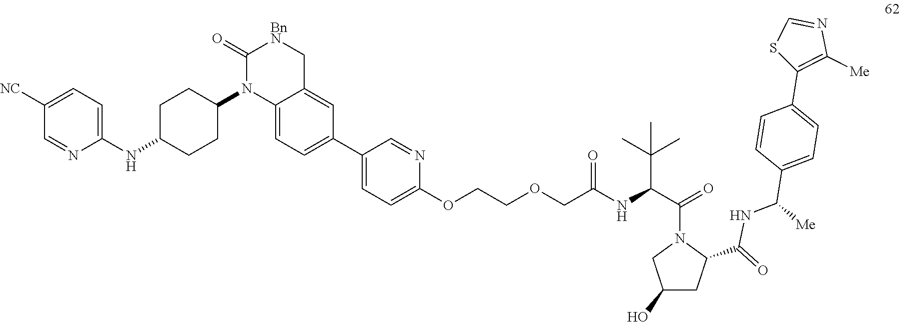

Following the preparation method for Formula 1, use intermediate eb1-1 instead of intermediate a0-1.

LC-MS (ESI) m/z (M+H)⁺ calculated for C₅₉H₆₇N₁₀O₇S⁺=1059.5; found 1059.6.

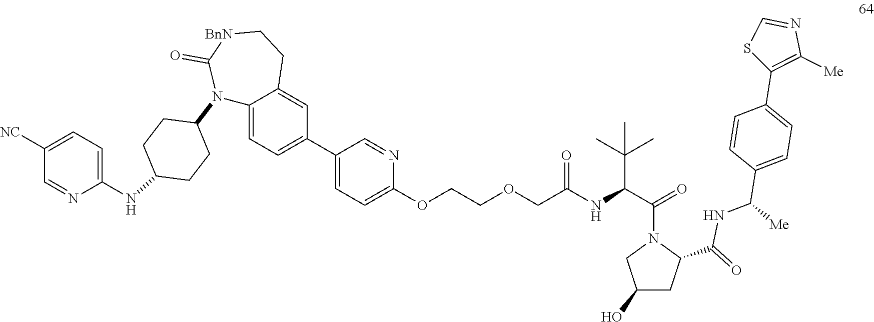

Following the preparation method for Formula 1, use intermediate eb2-1 instead of intermediate e0-1.

Wherein, the preparation method for Formula eb2-1 is by reference to Formula eb1-1, using intermediate 6bb-2 instead of intermediate 6bb; wherein, the preparation of Formula 6bb-2 is by reference to Formula 6bb, using 2-(5-bromo-2-nitrophenyl)acetonitrile instead of 2-nitro-5-bromobenzonitrile (Formula 1bb).

LC-MS (ESI) m/z (M+H)⁺ calculated for C₆₀H₆₉N₁₀O₇S⁺=1073.5; found 1073.6.

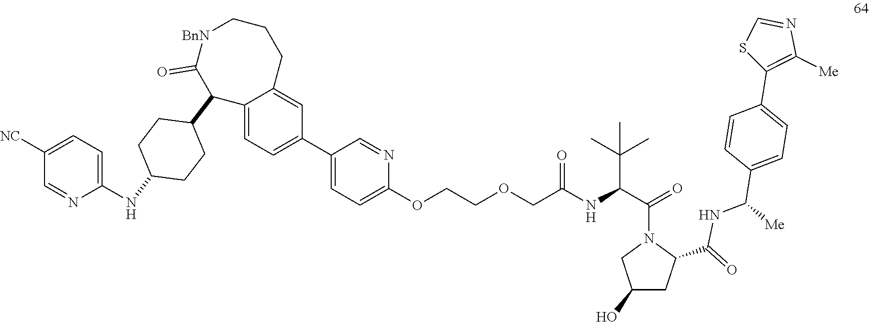

Following the preparation method for Formula 1, use intermediate eb3-1 instead of intermediate e0-1.

Wherein, the preparation method for Formula eb3-1 is by reference to Formula eb1-1, using intermediate 6bb-3 instead of intermediate 6bb; wherein, the preparation of Formula 6bb-3 is by reference to Formula 6bb, using 2-(5-bromo-2-nitrophenyl)propanenitrile instead of 2-nitro-5-bromobenzonitrile (Formula 1bb).

LC-MS (ESI) m/z (M+H)⁺ calculated for C₆₁H₇₁N₁₀O₇S⁺=1087.5; found 1087.6.

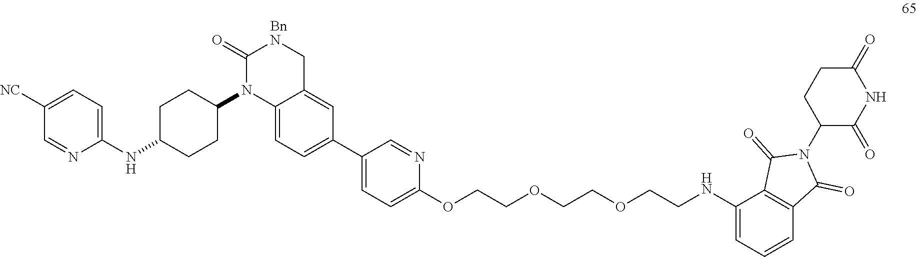

Following the preparation method for Formula 32, use intermediate ab1-2 instead of intermediate a0-1.

LC-MS (ESI) m/z (M+H)⁺ calculated for C₅₁H₅₂N₉O₈⁺=918.4; found 918.4.

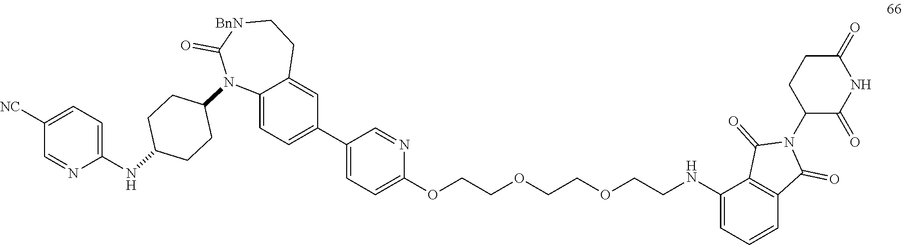

Following the preparation method for Formula 32, use intermediate ab2-2 instead of intermediate a0-1.

Wherein, the preparation method for Formula ab2-2 is by reference to Formula ab1-2, using intermediate 6bb-2 instead of intermediate 6bb; wherein, the preparation of Formula 6bb-2 is by reference to Formula 6bb, using 2-(5-bromo-2-nitrophenyl)acetonitrile instead of 2-nitro-5-bromobenzonitrile (Formula 1bb).

LC-MS (ESI) m/z (M+H)⁺ calculated for C₅₂H₅₄N₉O₈⁺=932.4; found 932.4.

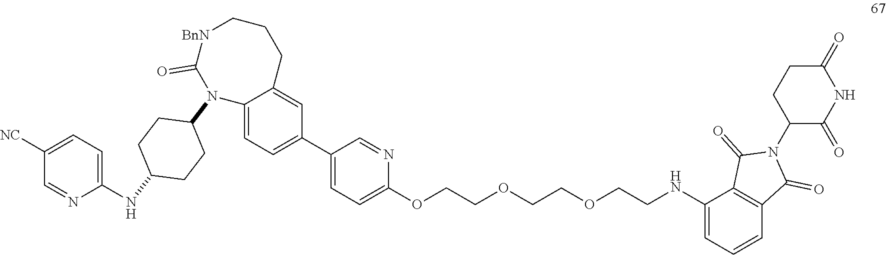

Following the preparation method for Formula 32, use intermediate ab3-2 instead of intermediate a0-1.

Wherein, the preparation method for Formula ab3-2 is by reference to Formula ab1-2, using intermediate 6bb-3 instead of intermediate 6bb; wherein, the preparation of Formula 6bb-3 is by reference to Formula 6bb, using 2-(5-bromo-2-nitrophenyl)propanenitrile instead of 2-nitro-5-bromobenzonitrile (Formula 1bb).

LC-MS (ESI) m/z (M+H)⁺ calculated for C₅₃H₅₆N₉O₈⁺=946.4; found 946.4.

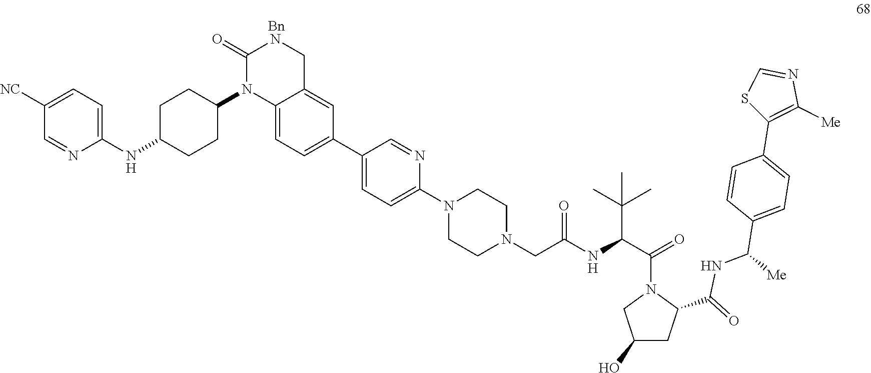

Following the preparation method for Formula 1, use intermediate ab1-0 instead of intermediate e0-1.

LC-MS (ESI) m/z (M+H)⁺ calculated for C₆₁H₇₁N₁₂O₅S⁺=1083.5; found 1083.6.

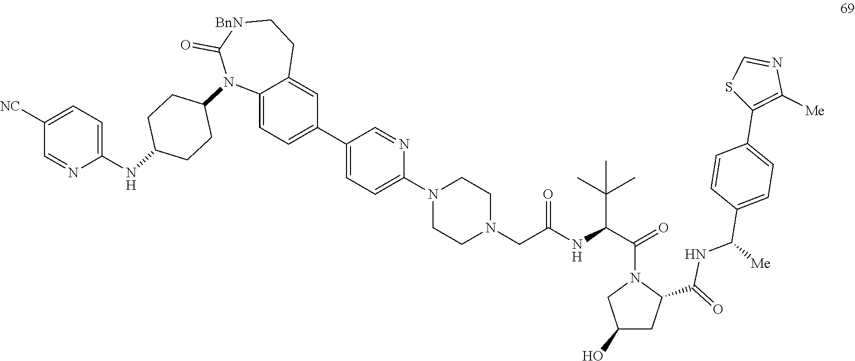

Following the preparation method for Formula 1, use intermediate hb2-0 instead of intermediate e0-1.

Wherein, the preparation method for Formula hb2-0 is by reference to Formula hb1-0, using intermediate 6bb-2 instead of intermediate 6bb; wherein, the preparation of Formula 6bb-2 is by reference to Formula 6bb, using 2-(5-bromo-2-nitrophenyl)acetonitrile instead of 2-nitro-5-bromobenzonitrile (Formula 1bb).

LC-MS (ESI) m/z (M+H)⁺ calculated for C₆₂H₇₃N₁₂O₅S⁺=1097.6; found 1097.6.

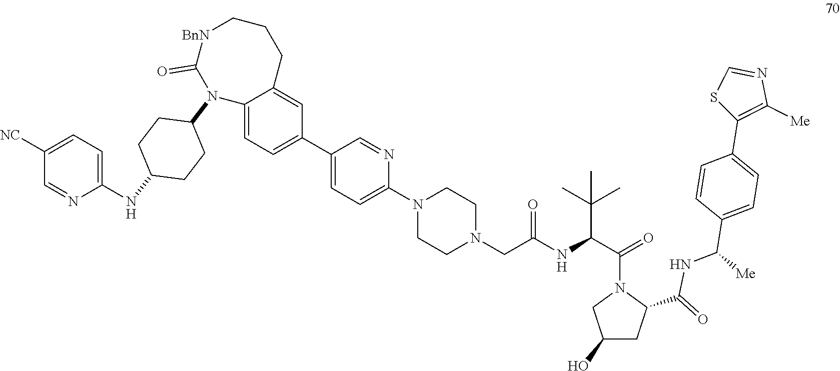

Following the preparation method for Formula 1, use intermediate hb3-0 instead of intermediate e0-1.

Wherein, the preparation method for Formula hb3-0 is by reference to Formula hb1-0, using intermediate 6bb-3 instead of intermediate 6bb; wherein, the preparation of Formula 6bb-3 is by reference to Formula 6bb, using 2-(5-bromo-2-nitrophenyl)propanenitrile instead of 2-nitro-5-bromobenzonitrile (Formula 1bb).

LC-MS (ESI) m/z (M+H)⁺ calculated for C₆₃H₇₅N₁₂O₅S⁺=1111.6; found 1111.6.

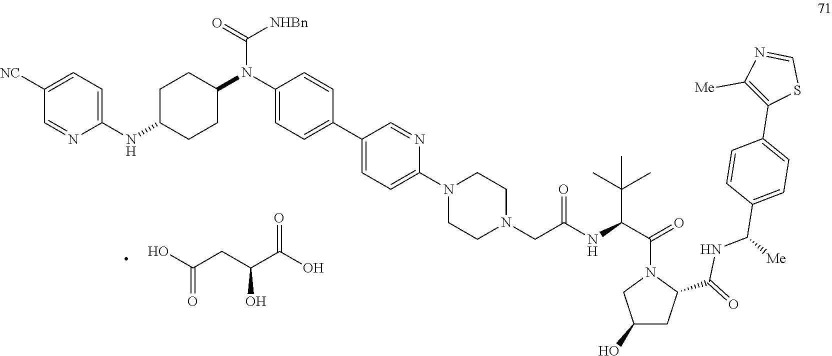

Dissolve 640 mg of the compound as shown in Formula 18 and 800 mg of L-malic acid in 5 mL of anhydrous ethanol. React overnight at room temperature. A white solid precipitates out, which is then filtered with suction to obtain 699 mg of the compound as shown in Formula 71, with a yield of 97%.

LC-MS (ESI) m/z (M+H)⁺ calculated for C₆₀H₇₁N₁₂O₅S⁺=1071.5; found 1071.6.

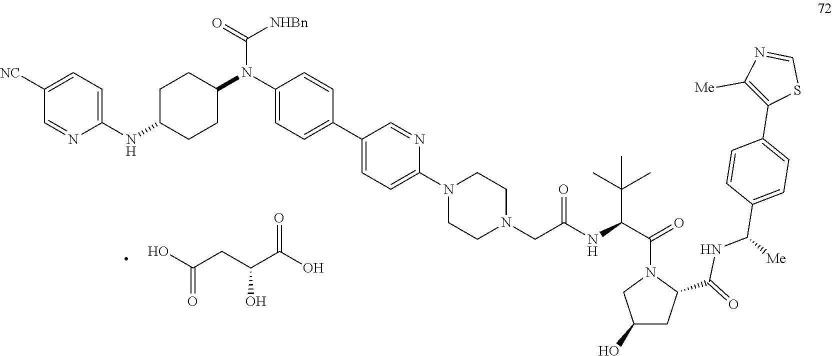

By reference to the preparation method for Formula 71, the starting materials are the compound as shown in Formula 18 and D-malic acid.

LC-MS (ESI) m/z (M+H)⁺ calculated for C₆₀H₇₁N₁₂O₅S⁺=1071.5; found 1071.6.

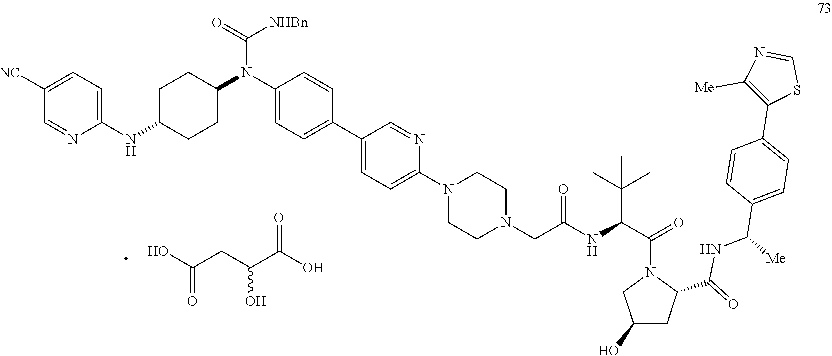

By reference to the preparation method for Formula 71, the starting materials are the compound as shown in Formula 18 and (±)-malic acid.

LC-MS (ESI) m/z (M+H)⁺ calculated for C₆₀H₇₁N₁₂O₅S⁺=1071.5; found 1071.6.

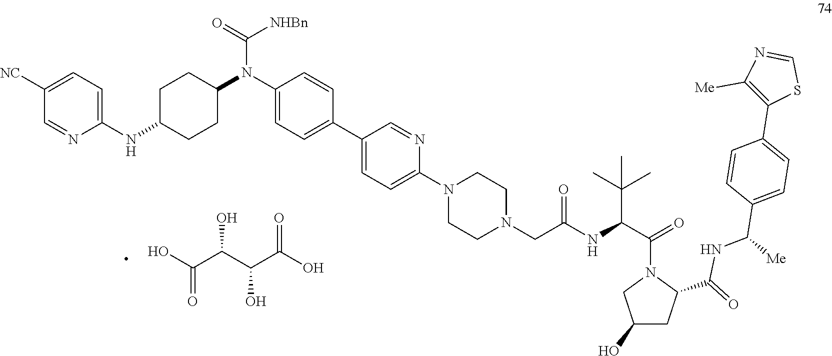

By reference to the preparation method for Formula 71, the starting materials are the compound as shown in Formula 18 and L-tartaric acid.

LC-MS (ESI) m/z (M+H)⁺ calculated for C₆₀H₇₁N₁₂O₅S⁺=1071.5; found 1071.6.

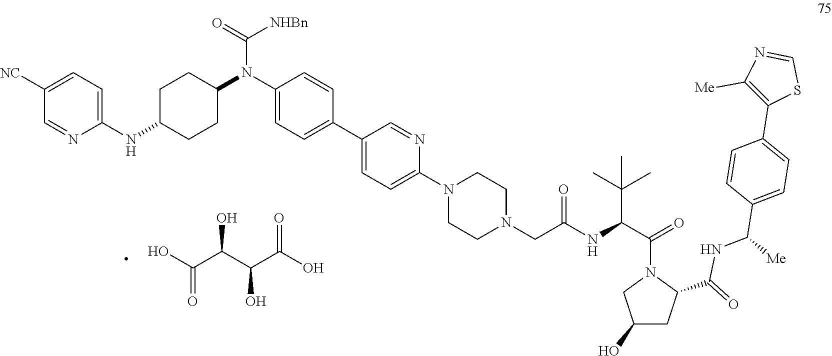

By reference to the preparation method for Formula 71, the starting materials are the compound as shown in Formula 18 and D-tartaric acid.

LC-MS (ESI) m/z (M+H)⁺ calculated for C₆₀H₇₁N₁₂O₅S⁺=1071.5; found 1071.6.

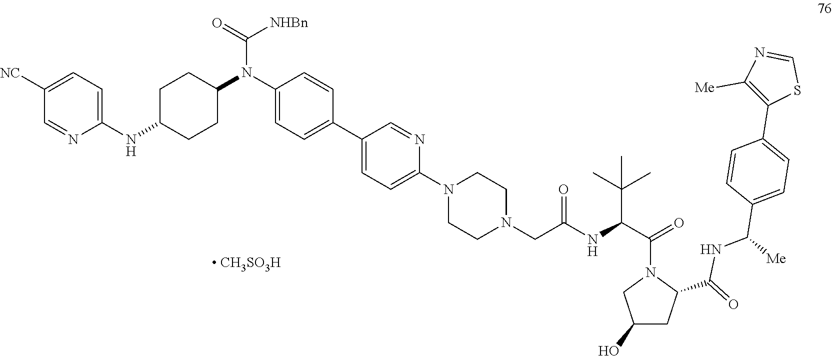

Following the preparation method for Formula 71, the starting materials are the compound as shown in Formula 18 and methanesulfonic acid.

LC-MS (ESI) m/z (M+H)⁺ calculated for C₆₀H₇₁N₁₂O₅S⁺=1071.5; found 1071.6.

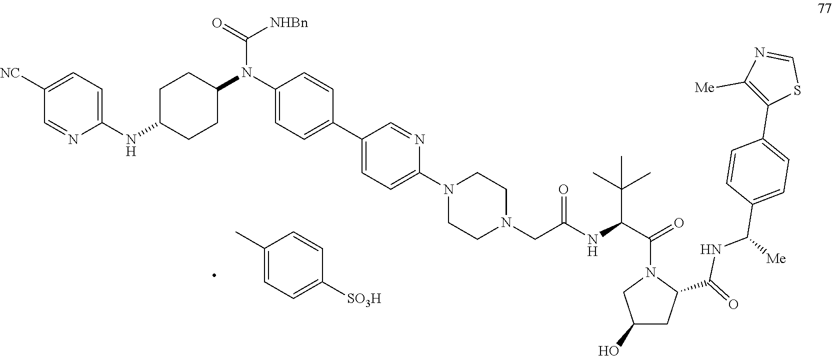

By reference to the preparation method for Formula 71, the starting materials are the compound as shown in Formula 18 and p-toluenesulfonic acid.

LC-MS (ESI) m/z (M+H)⁺ calculated for C₆₀H₇₁N₁₂O₅S⁺=1071.5; found 1071.6.

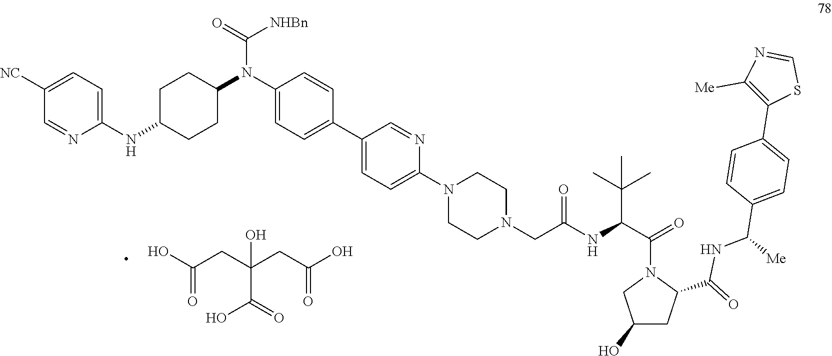

Following the preparation method for Formula 71, the starting materials are the compound as shown in Formula 18 and citric acid.

LC-MS (ESI) m/z (M+H)⁺ calculated for C₆₀H₇₁N₁₂O₅S⁺=1071.5; found 1071.6.

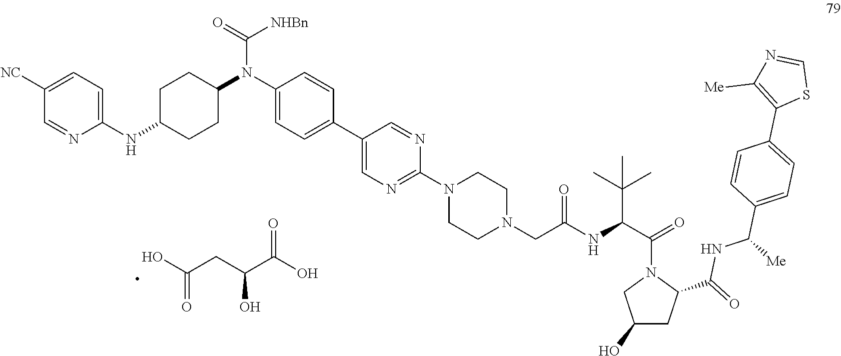

By reference to the preparation method for Formula 71, the starting materials are the compound as shown in Formula 45 and L-malic acid.

LC-MS (ESI) m/z (M+H)⁺ calculated for C₅₉H₇₀N₁₃O₅S⁺=1072.5; found 1072.6.

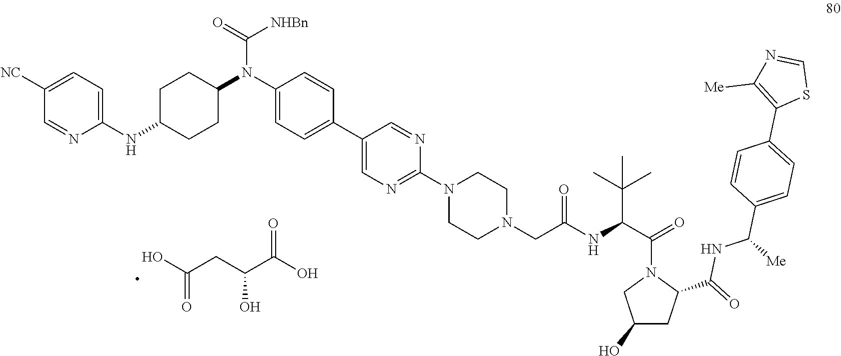

By reference to the preparation method for Formula 71, the starting materials are the compound as shown in Formula 45 and D-malic acid.

LC-MS (ESI) m/z (M+H)⁺ calculated for C₅₉H₇₀N₁₃O₅S⁺=1072.5; found 1072.6.

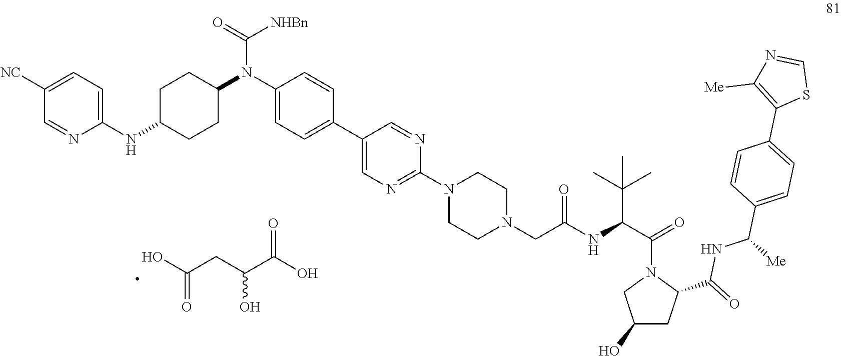

By reference to the preparation method for Formula 71, the starting materials are the compound as shown in Formula 45 and (±)-malic acid. LC-MS (ESI) m/z (M+H)⁺ calculated for C59H₇₀N₁₃O₅S⁺=1072.5; found 1072.6.

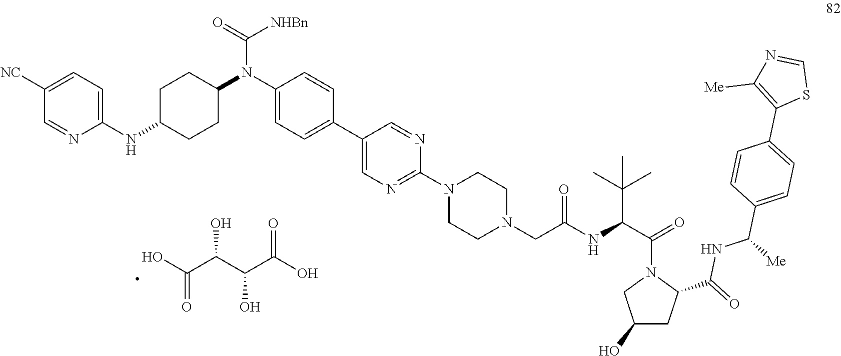

By reference to the preparation method for Formula 71, the starting materials are the compound as shown in Formula 45 and L-tartaric acid.

LC-MS (ESI) m/z (M+H)⁺ calculated for C₅₉H₇₀N₁₃O₅S⁺=1072.5; found 1072.6.

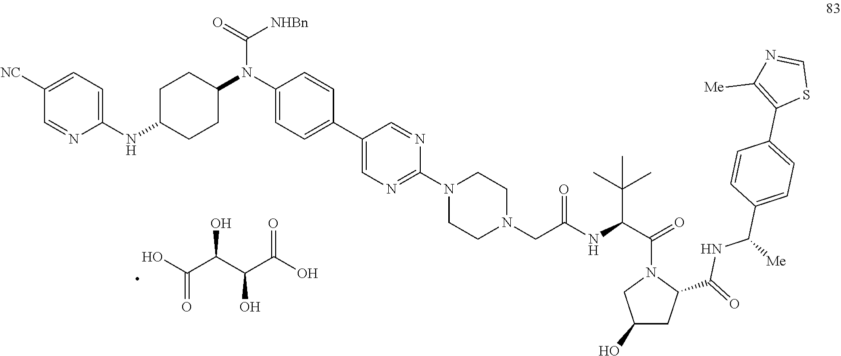

By reference to the preparation method for Formula 71, the starting materials are the compound as shown in Formula 45 and D-tartaric acid.

LC-MS (ESI) m/z (M+H)⁺ calculated for C₅₉H₇₀N₁₃O₅S⁺=1072.5; found 1072.6.

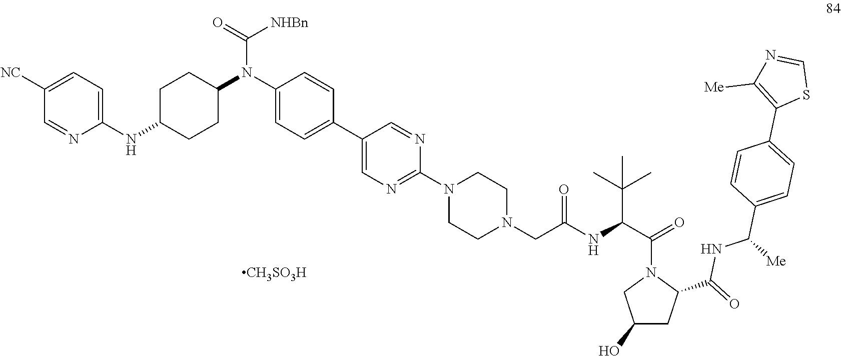

By reference to the preparation method for Formula 71, the starting materials are the compound as shown in Formula 45 and methanesulfonic acid.

LC-MS (ESI) m/z (M+H)⁺ calculated for C₅₉H₇₀N₁₃O₅S⁺=1072.5; found 1072.6.

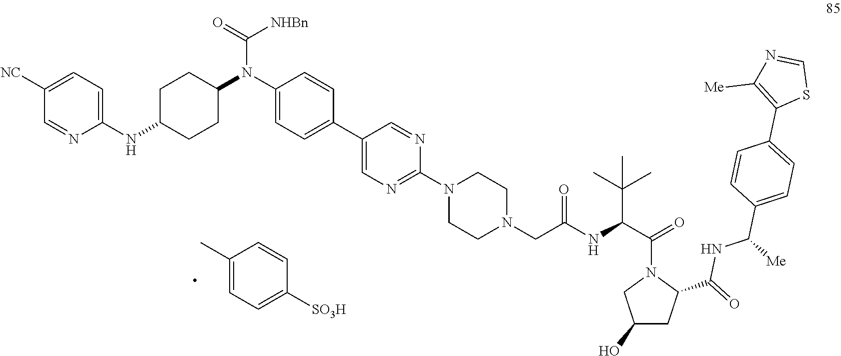

By reference to the preparation method for Formula 71, the starting materials are the compound as shown in Formula 45 and p-toluenesulfonic acid.

LC-MS (ESI) m/z (M+H)⁺ calculated for C₅₉H₇₀N₁₃O₅S⁺=1072.5; found 1072.6.

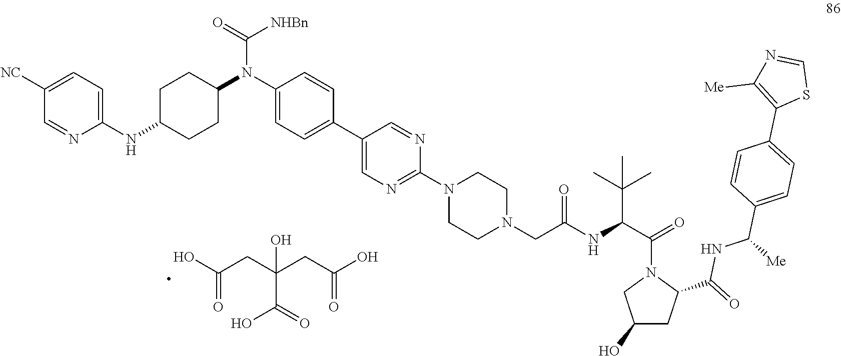

By reference to the preparation method for Formula 71, the starting materials are the compound as shown in Formula 45 and citric acid.

LC-MS (ESI) m/z (M+H)⁺ calculated for C₅₉H₇₀N₁₃O₅S⁺=1072.5; found 1072.6.

### Example 2: Biological Activity Test of the Small Molecule Conjugates as Shown in Formulae 1 to 86

### I. Validation of CDK12/13 Degradation Effect and Other Related Protein Degradation Effects via Western Blot

Western blot was used to determine the degradation of CDK12/13 protein in leukemia cells KASUMI-1, MV4-11, and Jurkat. The two compounds with the best degradation effects were further validated for their ability to degrade CDK12/13 protein in breast cancer cells MDA-MB-231 using a concentration gradient.

### Experimental Materials:

Cell lines: KASUMI-1, MV4-11, Jurkat, or MDA-MB-231
Culture medium: DMEM/RIPM-1640+10% Hyclone serum
Compound dissolution method: Compounds were dissolved in DMSO to prepare a 50 mM stock solution, and then diluted to the corresponding concentrations.

### 1. In Vitro Cell Culture and Drug Administration (for KASUMI-1, MV4-11, MDA-MB-231, Jurkat, or HCT116)

(1) In vitro cell culture: The selected cells were cultured in a 37 °C constant temperature incubator with 5% CO₂. Once the cell density reached 70-90%, they were passaged for subsequent experiments.

(2) Cell seeding: Cells were digested, and the cell suspension was transferred to a 1.5 mL Eppendorf tube. After centrifugation at 1,000 rpm for 5 min, the cells were resuspended in 2 mL of culture medium and counted. The cell suspension was seeded in a 24-well plate at a density of 200,000 cells per well and allowed to adhere for 8-12 h.

(3) Compound treatment: The 50 mM compound stock solution was diluted with cell culture medium to a final concentration of 0.01 μM-3 μM. Cells were incubated in a 37 °C incubator with 5% CO2 for 3 h-48 h. The DMSO group served as the negative control.

### 2. Cell Lysis

At the end of the treatment time, the cells were collected and washed once with PBS. An appropriate volume of 4% SDS was added to lyse the cells, and the mixture was sonicated until no longer viscous. After centrifugation at 12,000 rpm at room temperature for 30 min, the supernatant was transferred to a new EP tube for protein quantification.

### 3. Protein Quantification

A 2 mg/mL BSA standard was serially diluted by half to create a standard curve at the following concentrations: 2 mg/mL, 1 mg/mL, 0.5 mg/mL, 0.25 mg/mL, 0.125 mg/mL, and 0.0625 mg/mL. A and B solutions from the BCA quantification kit were mixed at a volume ratio of 50:1 (calculating for 200 μL of A solution and 4 μL of B solution per sample). 10 μL of the BSA standards and the samples from step 2 were added to a 96-well plate, followed by 200 μL of the mixed A and B solutions. After gentle mixing, the plate was incubated in the dark at 37 °C for 30 min. Absorbance values were measured at 562 nm, and sample protein concentrations were calculated using the standard curve.

### 4. Western Blot

### (1) Protein Sample Preparation

An amount corresponding to 20 μg of protein from the samples obtained in step 2 was transferred to a new EP tube. 10 μL of 6× Loading Buffer was added, and the total volume was adjusted to 30 μL with distilled water. The samples were denatured by heating at 95 °C for 10 min, and after cooling, they were centrifuged and mixed before the Western blot experiment. The remaining samples were stored at −80 °C.

### (2) Gel Casting

a. Separating gel: The concentration of the separating gel was chosen based on the molecular weight of the target protein. The gel clamp was first assembled by clamping two glass plates together on a flat surface. The separating gel reagents were added sequentially to a centrifuge tube and mixed by vortexing. The mixture was then poured between the glass plates, and an appropriate amount of isopropanol was added to the top of the gel. The gel was allowed to solidify.

b. Stacking gel: The isopropanol was discarded from the top of the separating gel, and the surface was washed with triple-distilled water. The stacking gel reagents were prepared and mixed by vortexing. The mixture was then poured between the glass plates, a comb was inserted, and the gel was allowed to solidify.

### (3) Electrophoresis

a. Preparation: The prepared gel clamp was installed in the electrophoresis tank and placed in the electrophoresis apparatus. A diluted 1× Tricine buffer was added to the inner tank, and a 1× Running buffer was added to the outer tank. The setup was left to stand for several minutes.

b. Sample loading: The samples from step 4 (1) were mixed by vortexing, and a certain amount of each sample (5-10 μL) was loaded into the wells. After loading, the electrophoresis apparatus was turned on. The initial setting was a constant voltage of 70 V for 10 min. After confirming the current was normal and the samples had entered the separating gel, the voltage was adjusted to 130 V. Electrophoresis was stopped once the samples had run to the appropriate position.

### (4) Membrane Transfer and Blocking

a. Transfer: In the transfer clamp, a sponge, a thick filter paper, and a thin filter paper were added in sequence. The gel was then carefully removed from the clamp using a gel spatula to cut away the stacking gel and placed in the center of the filter paper. A PVDF membrane, pre-activated with methanol, was placed on the gel from one side, and air bubbles were removed. A thin filter paper, a thick filter paper, and a sponge were then added in sequence, and the clamp was secured. The transfer assembly was placed in the transfer tank. An appropriate amount of transfer buffer was added to the tank, and ice packs were placed in the outer layer of the tank to prevent heat from affecting the transfer.

b. Blocking: After transfer, the clamp was opened, and the gel and PVDF membrane were removed (if the transfer was successful, the protein ladder would be entirely transferred to the PVDF membrane). The PVDF membrane was then cut according to the molecular weight of the target protein, placed in 5% skim milk, and blocked at room temperature for 60 min.

### (5) Antibody Incubation

a. Primary antibody incubation: After blocking, the milk was discarded, and the PVDF membrane was washed 3 times with TBST (15 min, 5 min, 5 min). An appropriate amount of primary antibody was added (typically diluted 1:1000 with antibody diluent) and incubated overnight on a shaker at 4 °C.

b. Secondary antibody incubation: The primary antibody was recovered, and the membrane was washed 3 times with TBST (15 min, 5 min, 5 min). Secondary antibody was then added (typically prepared at a 1:5000 dilution in 5% skim milk) and incubated at room temperature for 60 min on a horizontal shaker.

### (6) Exposure

a. Washing: The secondary antibody was discarded, and the membrane was washed 3 times with TBST (15 min, 5 min, 5 min).

b. Exposure: The ECL detection reagent was prepared by mixing solution A and solution B at a 1:1 ratio. The residual TBST on the membrane was blotted with filter paper, and the membrane was transferred to an exposure plate with forceps. The ECL detection solution was dropped onto the membrane to cover it, and it was left to stand for about 1 min. The plate was then placed in an AI800 exposure machine, and an appropriate exposure time was selected.

### Degradation Rate Calculation Method:

The exposure results were quantitatively analyzed using Image J software. First, the grayscale values of the bands were calculated using Image J. Then, the grayscale value of the target protein was divided by the grayscale value of the internal reference protein for normalization. The calculation formula is:

$${D}_{experimental} = \{ 1 - [ ( {A}_{experimental , target} / {A}_{experimental , internal} ) \div ( {A}_{negativecontrol , target} / {A}_{negativecontrol , internal} ) ] \} \times 100 \%$$

Where A is the grayscale value and D is the degradation rate.

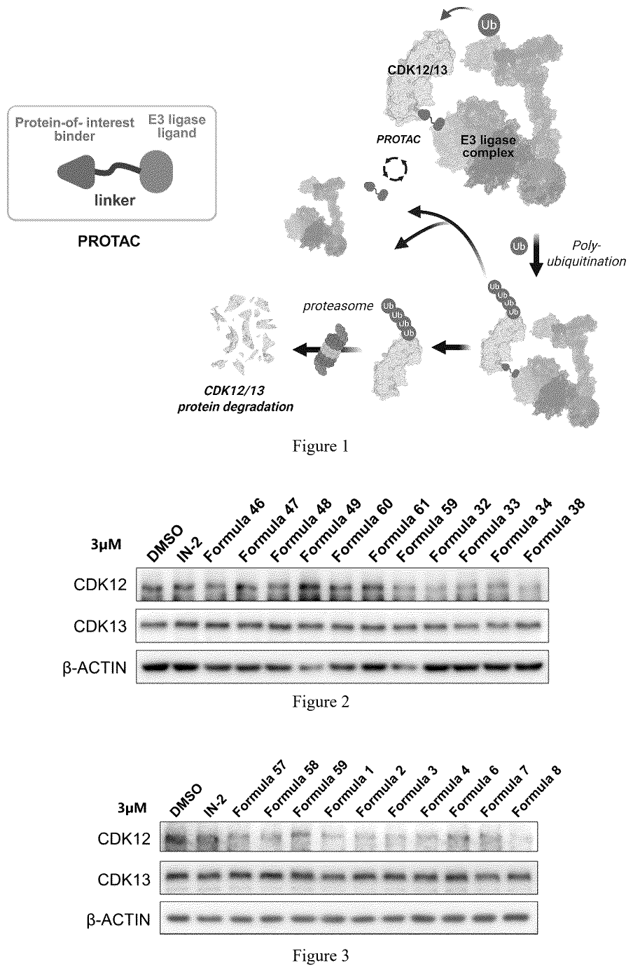

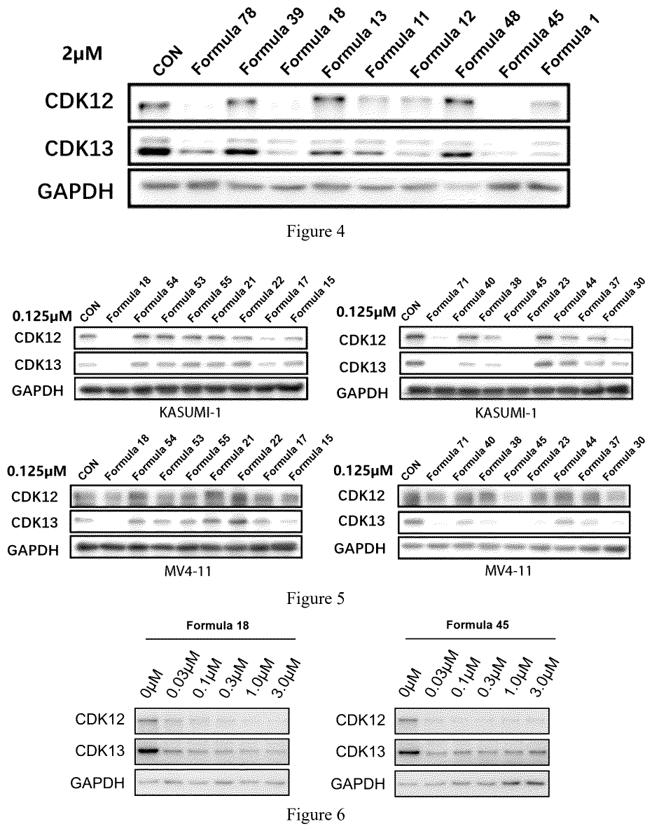

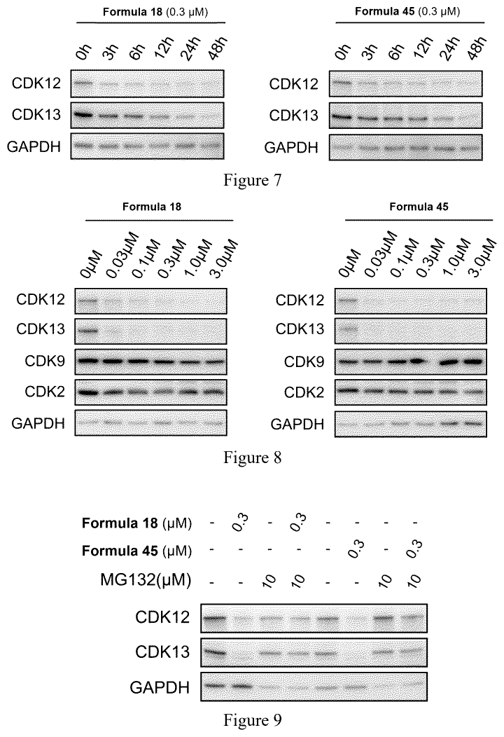

The degradation activity of the small molecule conjugates of the present invention on CDK12 and CDK13 is shown in FIGS. 2-9. In the figures, CDK12 and CDK13 are the target proteins under study, and β-Actin and GAPDH are the internal reference proteins. Internal reference proteins are used to indicate the total protein amount in each experimental group for quantitative analysis.

From the test results in FIGS. 2-9, it can be seen that most of the conjugates can degrade CDK12/13 at a certain concentration, and the compound as shown in Formula 45 has the strongest degradation effect.

TABLE 1
Degradation activity of the compound as shown
in Formula 45 on CDK12 and CDK13

| | | CDK12 | CDK13 |
|---|---|---|---|
| Cell line | Cell type | DC₅₀ | DC₅₀ |
| MDA-MB-231 | Breast cancer cells | 29 nM | 18 nM |
| HCT116 | Colorectal cancer cells | 34 nM | 24 nM |
| KASUMI-1 | Leukemia cells | 28 nM | 17 nM |
| MV4-11 | Leukemia cells | 30 nM | 21 nM |

The list of example compounds, their aliases, an their activity can be seen in Table 2 below. In the table, + represents the potency of degradation, − represents no degradation observed, and N.D. represents not determined.)

TABLE 2
List of Example Compounds, Aliases, and Activity

| | CDK12 | CDK13 |
|---|---|---|
| | degradation | degradation |
| Formula | Activity | Activity |
| 1 | + + + + | + + + + |
| 2 | + + | + + + |
| 3 | + | + |
| 4 | + | + |
| 5 | N.D. | N.D. |
| 6 | + + | + + + |
| 7 | + + | + |
| 8 | + + + | + + + |
| 9 | + | + |
| 10 | + + | + + |
| 11 | + + + | + + + + |
| 12 | + + + | + + + + |
| 13 | + | + |
| 14 | + | − |
| 15 | − | − |
| 16 | + | + |
| 17 | + + | + + + |
| 18 | + + + + + | + + + + + |
| 19 | − | − |
| 20 | + | + |
| 21 | + + | + + |
| 22 | − | − |
| 23 | + | − |
| 24 | − | − |
| 25 | + | + |
| 26 | − | − |
| 27 | + | − |
| 28 | + | + |
| 29 | + | − |
| 30 | + + + | + + + |
| 31 | + + | + |
| 32 | + + | + + + |
| 33 | + + + | + + |
| 34 | + + | + |
| 35 | + + | + |
| 36 | − | − |
| 37 | + + | + + + |
| 38 | + + + | + + |
| 39 | + + | + |
| 40 | + + + | + + |
| 41 | + + | + + |
| 42 | + + | + + |
| 43 | + + | + + |
| 44 | + + + | + + + + |
| 45 | + + + + + | + + + + + |
| 46 | + + | + + + |
| 47 | + + + | + + |
| 48 | + | − |
| 49 | + + + | + + |
| 50 | + | + |
| 51 | + | + |
| 52 | + | − |
| 53 | + | + + |
| 54 | − | − |
| 55 | − | − |
| 56 | − | − |
| 57 | − | − |
| 58 | − | − |
| 59 | + + | + + |
| 60 | + + | + + + |
| 61 | − | − |
| 62 | + + + | + + + |
| 63 | N.D. | N.D. |
| 64 | N.D. | N.D. |
| 65 | + + | + + |
| 66 | N.D. | N.D. |
| 67 | N.D. | N.D. |
| 68 | + + | + + |
| 69 | + + + | + + + + |
| 70 | + + + + + | + + + + + |
| 71 | + + + + + | + + + + + |
| 72 | + + + + + | + + + + + |
| 73 | + + + + + | + + + + + |
| 74 | + + + + + | + + + + + |
| 75 | + + + + + | + + + + + |
| 76 | + + + + + | + + + + + |
| 77 | + + + + + | + + + + + |
| 78 | + + + + + | + + + + + |
| 79 | + + + + + | + + + + + |
| 80 | + + + + + | + + + + + |
| 81 | + + + + + | + + + + + |
| 82 | + + + + + | + + + + + |
| 83 | + + + + + | + + + + + |
| 84 | + + + + + | + + + + + |
| 85 | + + + + + | + + + + + |
| 86 | + + + + + | + + + + + |

+ represents the degradation effect on the protein, and more + symbols indicate a more significant degradation.
− represents no degradation of the protein.
Specifically:
+ + + + + represents DC₅₀ < 100 nM
+ + + + represents 100 nM < DC₅₀ < 0.5 μM
+ + + represents 0.5 μM < DC₅₀ < 1 μM
+ + represents 1 μM < DC₅₀ < 3 μM
+ represents 3 μM < DC₅₀ < 10 μM
− represents no degradation activity

### II. Staining-Based Detection of the Proliferation Inhibitory Effect of Compounds on Tumor Cells

The proliferation inhibition of compounds on leukemia cell lines MV4-11 and KASUMI-1 was determined using the CCK8 assay, while the inhibition on solid tumor cell lines MDA-MB-231 was determined using the Sulforhodamine B (SRB) assay.

CCK8 Assay: First, MV4-11 and KASUMI-1 cells were seeded in a 96-well plate at a density of 5,000 cells per well. Then, a concentration gradient (0.03 μM-10 μM) of the compounds was administered for 72 h, with DMSO solvent serving as the negative control. At the end of the treatment, 10 μL of CCK8 reagent was added to each well. The plate was incubated for 4 hours at 37 °C in the dark. After mixing, the absorbance value was measured at 450 nm.

SRB Assay: First, MDA-MB-231, L-02, and HK-2 cells were seeded in a 96-well plate at a density of 3,000 cells per well. After 8-12 h of cell adhesion, a concentration gradient (62.5 nM-10 μM) of the compounds was administered for 72 h, with DMSO solvent serving as the negative control. At the end of the treatment, the cells were washed once with 70 μL of PBS, then fixed with 70 μL of 10% trichloroacetic acid at 4 °C for 1 h. After rinsing with tap water and drying in an oven, 70 μL of SRB staining solution was added to each well for 20 min at room temperature. The staining solution was then discarded, and the cells were rinsed with 1% glacial acetic acid and dried in an oven. Finally, 150 μL of 10 mM Tris-base dissolving solution was added, and the plate was shaken for 15 min before the absorbance value was measured at 540 nm.

Methodology for Combination Drug Use: MD-MB-231 cells were seeded in a 6-well plate at a density of 2,000 cells per well. After 24 h of cell adhesion, the cells were treated with a specific concentration (0.5 μM, 1 μM) of the PARP inhibitor Olaparib in combination with a specific concentration (0.5 μM, 1 μM) of the compound of Formula 18 for 10 days. The solvent DMSO served as the negative control. At the end of the treatment, the cells were washed once with 100 μL of PBS, then fixed with 700 μL of 10% trichloroacetic acid at 4 °C for 1 h. After rinsing with tap water and drying in an oven, 70 μL of SRB staining solution was added for 20 min at room temperature. The staining solution was discarded, and the cells were rinsed with 1% glacial acetic acid and dried in an oven. The plate was then photographed, and the number of cell clones in each group was counted using Image J software.

The calculation formula for cell proliferation inhibition rate from CCK8 and SRB assays is:

$${R}_{experimental} = ( 1 - {OD}_{experimental} / {OD}_{negative control} ) \times 100 \% ,$$

where OD is the absorbance value and R is the cell proliferation inhibition rate.

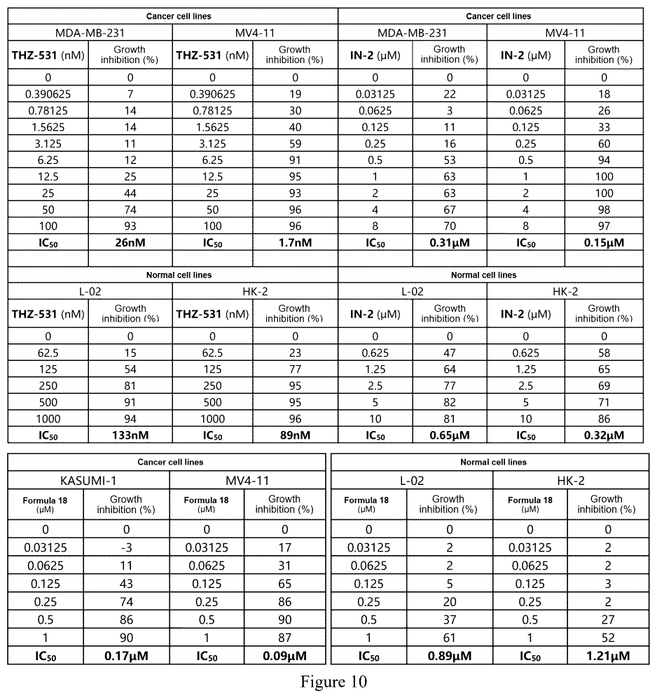

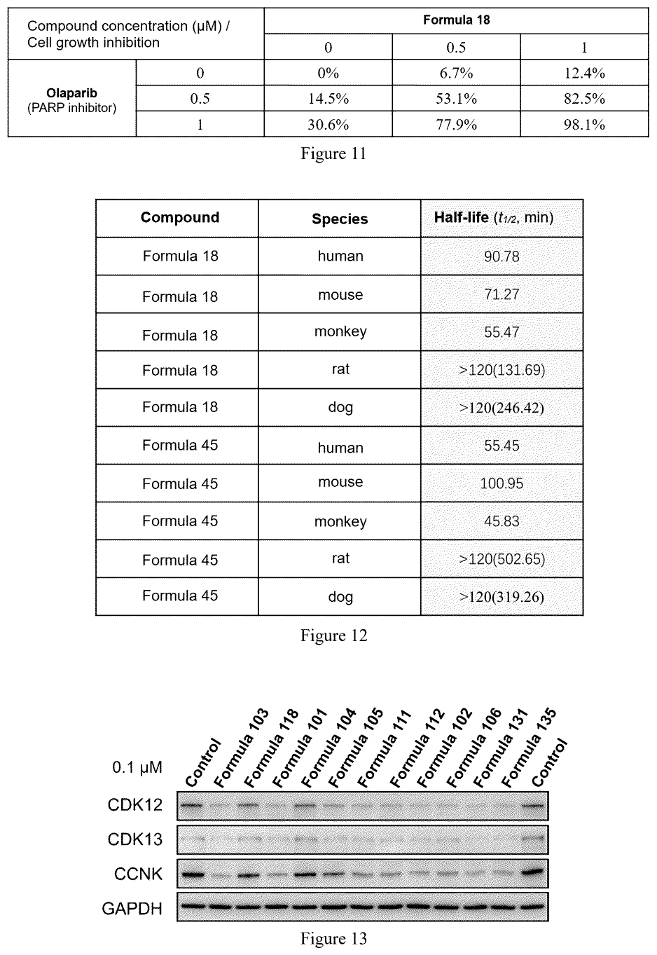

The calculation formula for cell proliferation inhibition rate from combination drug use is:

$${R}_{experimental} = ( 1 - {K}_{experimental} / {K}_{negative control} ) \times 100 \% ,$$

where K is the cell clone count and R is the cell proliferation inhibition rate. The results are shown in FIGS. 10 and 11.

As shown in FIG. 10, the small molecule conjugates provided in this patent application selectively inhibit the proliferation of tumor cells KASUMI-1 and MV4-11, while THZ-531 (a classic CDK12/13 inhibitor) and IN-2 (parent core molecule) exhibit indiscriminate proliferation inhibitory activity on both normal and tumor cells.

As shown in FIG. 11, the combination therapy of the small molecule conjugate described in this patent application and a PARP inhibitor can effectively and synergistically enhance the proliferation inhibitory activity on breast cancer cells MDA-MB-231, demonstrating a significant additive and complementary effect with existing clinical drugs.

### III Compound Liver Microsome Stability Test

### Experimental Materials:

Compounds: Ketanserin, Formula 18, Formula 45
Liver microsomes: Human liver microsomes (purchased from BioreclamationIVT); Dog, mouse, rat, and monkey liver microsomes (purchased from Xenotech)

Preheat PBS to a pH of 7.4.

Preparation of 500 μM compound solution: Add 5 μL of 10 mM compound solution and 95 μL of DMSO to an EP tube.

Preparation of solution containing 0.75 mg/mL liver microsomes and 1.5 μM compound: Add 1.5 μL of the 500 μM compound solution, 18.75 μL of the 20 mg/mL liver microsome solution, and 479.75 μL of PBS to an EP tube.

Preparation of 6 mM NADPH solution: Dissolve 5 mg of NADPH in 1 mL of PBS solution.

Add 30 μL of the solution containing 0.75 mg/mL liver microsomes and a concentration gradient of the compound to a 96-well plate. Add 15 μL of the 6 mM NADPH solution and 150 μL of a 1:1 acetonitrile:methanol solution containing an internal standard (IS). Vortex and centrifuge at 6,000 rpm for 10 min. 80 μL of the supernatant from each concentration sample is transferred to a 96-well plate containing 140 μL of pure water for LC/MS detection (injection volume of 10 μM). A standard curve is plotted based on the final compound concentrations and the corresponding mass spectrometry peak areas.

30 μL of the solution containing 0.75 mg/mL liver microsomes and 1.5 μM compound is added to a 96-well plate for detection at different time points (0, 5, 15, 30, and 45 minutes), with two replicates for each time point.

For the 0 min group, 150 μL of the 1:1 acetonitrile:methanol solution containing IS is added to the wells, followed by 15 μL of the 6 mM NADPH solution. The mixture is vortexed and centrifuged at 6,000 rpm for 10 min.

For the 5, 15, 30, and 45 min groups, the reaction is initiated by adding 15 μL of the 6 mM NADPH solution to the wells. After 5, 15, 30, and 45 minutes, 50 μL of the 1:1 acetonitrile:methanol solution containing IS is added to the corresponding wells to quench the reaction. The mixtures are vortexed and centrifuged at 6,000 rpm for 10 min.

80 μL of the supernatant from each well is transferred to a 96-well plate containing 140 μL of pure water for LC/MS detection (injection volume of 10 μM). The compound concentration value is determined by comparing the corresponding mass spectrometry peak area with the standard curve.

### Calculation Formulae:

Elimination rate K=(Inc₁−Inc₂)/(t₂−t₁) (where t₁ and t₂ are time points, and c₁ and c2 are the drug concentrations at times t₁ and t₂, respectively).

$$Half - life {t}_{1 / 2} = 0.6 \; 93 / K$$

The results are shown in FIG. 12.

As shown in FIG. 12, Formula 18 and Formula 45 both exhibit good liver microsome stability with a half-life greater than 45 min after co-incubation with liver microsomes from four species: human, rat, dog, and monkey.

### Preparation and Performance Testing of Compounds 101-172:

TABLE 3
Structures of compounds 101-172

| | |
|---|---|
| 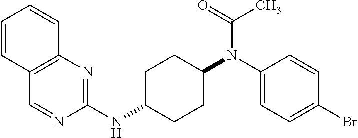 [SMILES: CC(=O)N(c1ccc(Br)cc1)[C@H]1CC[C@H](Nc2ncc3ccccc3n2)CC1] | Intermediate 1 |
| 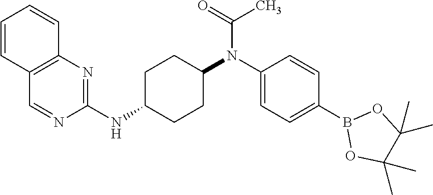 [SMILES: CC(=O)N(c1ccc(B2OC(C)(C)C(C)(C)O2)cc1)[C@H]1CC[C@H](Nc2ncc3ccccc3n2)CC1] | Intermediate 2 |
| 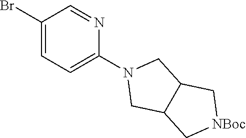 [SMILES: CC(C)(C)OC(=O)N1CC2CN(c3ccc(Br)cn3)CC2C1] | Intermediate 3 |
| 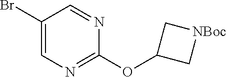 [SMILES: CC(C)(C)OC(=O)N1CC(Oc2ncc(Br)cn2)C1] | Intermediate 4 |
| 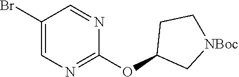 [SMILES: CC(C)(C)OC(=O)N1CC[C@H](Oc2ncc(Br)cn2)C1] | Intermediate 5 |
| 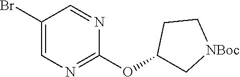 [SMILES: CC(C)(C)OC(=O)N1CC[C@@H](Oc2ncc(Br)cn2)C1] | Intermediate 6 |
| 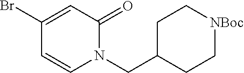 [SMILES: CC(C)(C)OC(=O)N1CCC(Cn2ccc(Br)cc2=O)CC1] | Intermediate 7 |
| 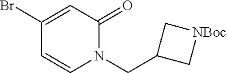 [SMILES: CC(C)(C)OC(=O)N1CC(Cn2ccc(Br)cc2=O)C1] | Intermediate 8 |
| 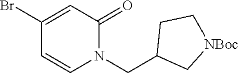 [SMILES: CC(C)(C)OC(=O)N1CCC(Cn2ccc(Br)cc2=O)C1] | Intermediate 9 |
| 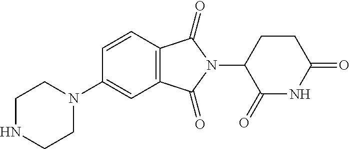 [SMILES: O=C1CCC(N2C(=O)c3ccc(N4CCNCC4)cc3C2=O)C(=O)N1] | Intermediate 10 |
| 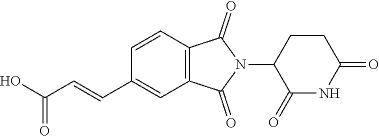 [SMILES: O=C(O)/C=C/c1ccc2c(c1)C(=O)N(C1CCC(=O)NC1=O)C2=O] | Intermediate 11 |
| 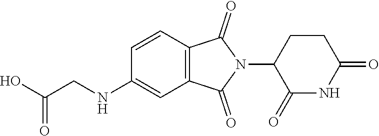 [SMILES: O=C(O)CNc1ccc2c(c1)C(=O)N(C1CCC(=O)NC1=O)C2=O] | Intermediate 12 |
| 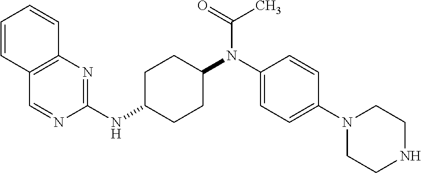 | Intermediate 13 |
| 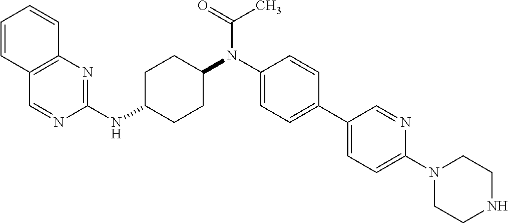 | Intermediate 14 |
| 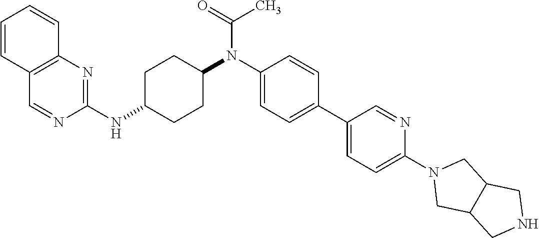 | Intermediate 15 |
| 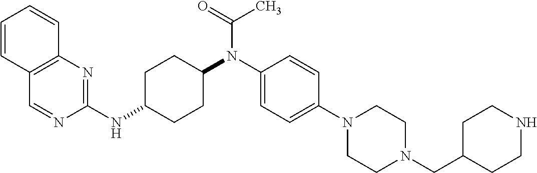 | Intermediate 16 |
| 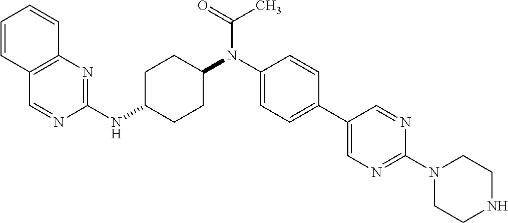 | Intermediate 17 |
| 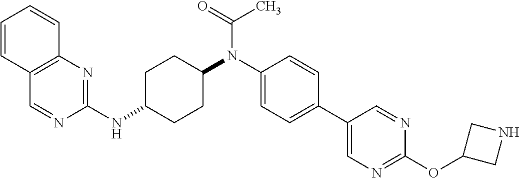 | Intermediate 18 |
| 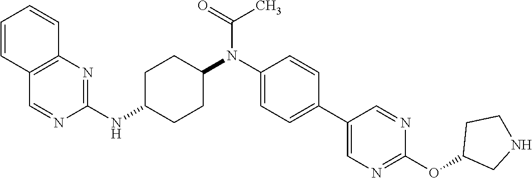 | Intermediate 19 |
| 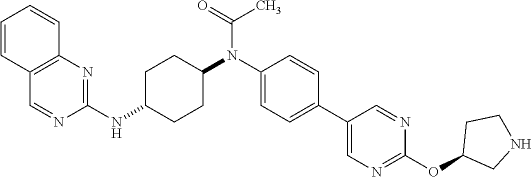 | Intermediate 20 |
| 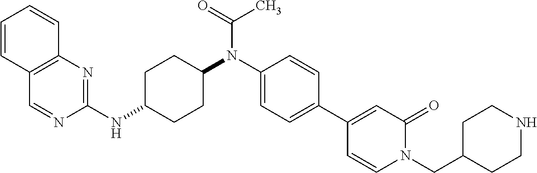 | Intermediate 21 |
| 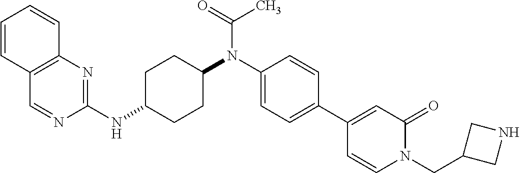 | Intermediate 22 |
| 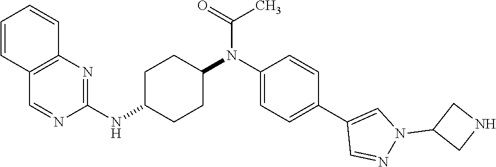 | Intermediate 23 |
| 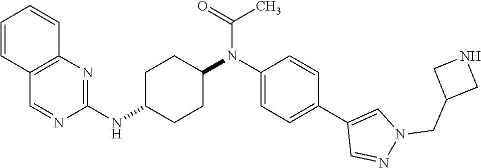 | Intermediate 24 |
| 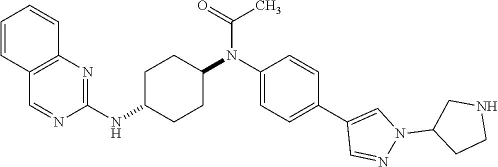 | Intermediate 25 |
| 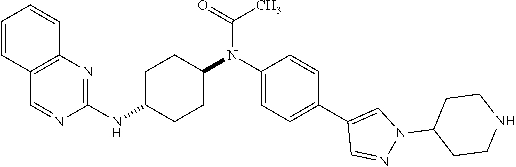 | Intermediate 26 |
| 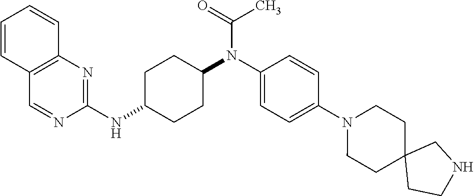 | Intermediate 27 |
| 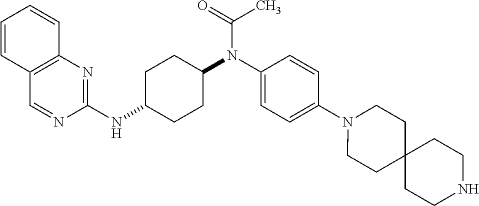 | Intermediate 28 |
| 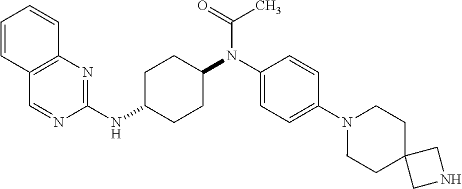 | Intermediate 29 |
| 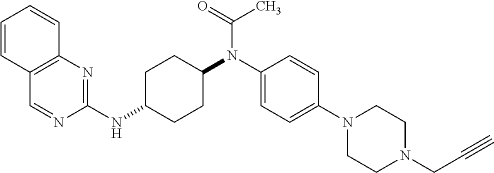 | Intermediate 30 |
| 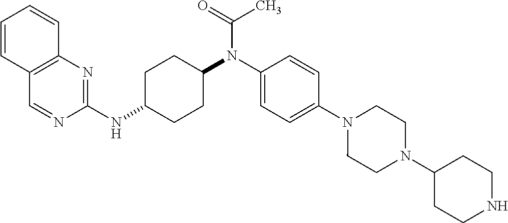 | Intermediate 31 |
| 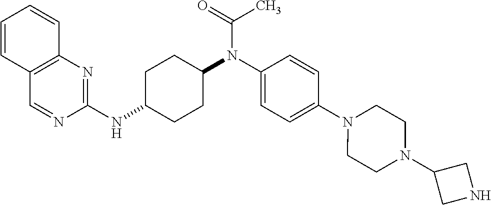 | Intermediate 32 |
| 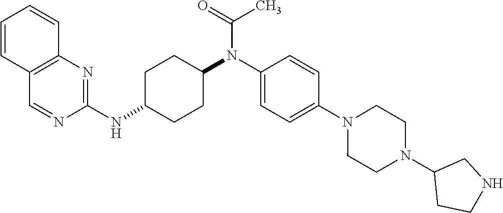 | Intermediate 33 |
| 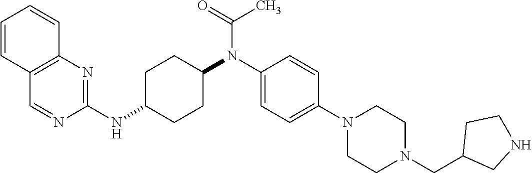 | Intermediate 34 |
| 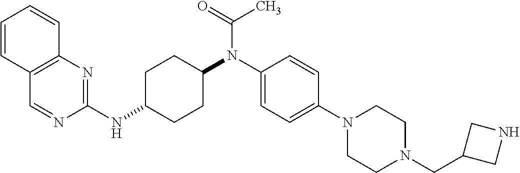 | Intermediate 35 |
| 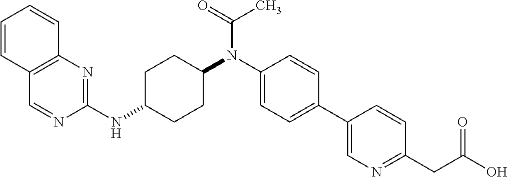 | Intermediate 36 |
| 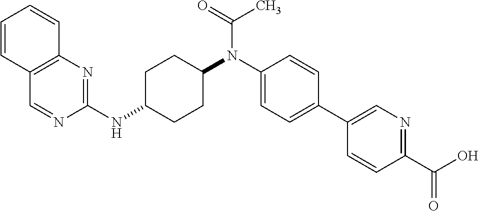 | Intermediate 37 |
| 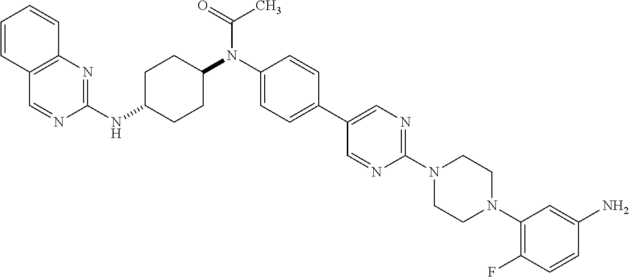 | Intermediate 38 |
| 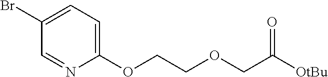 [SMILES: CC(C)(C)OC(=O)COCCOc1ccc(Br)cn1] | Intermediate 39 |
| 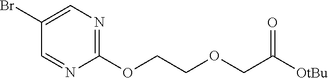 [SMILES: CC(C)(C)OC(=O)COCCOc1ncc(Br)cn1] | Intermediate 40 |
| 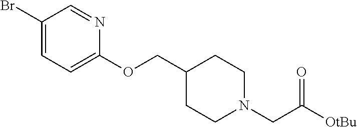 [SMILES: CC(C)(C)OC(=O)CN1CCC(COc2ccc(Br)cn2)CC1] | Intermediate 41 |
| 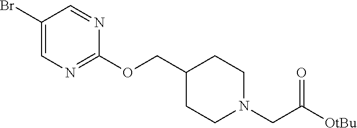 [SMILES: CC(C)(C)OC(=O)CN1CCC(COc2ncc(Br)cn2)CC1] | Intermediate 42 |
| 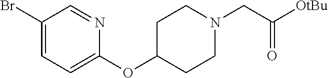 [SMILES: CC(C)(C)OC(=O)CN1CCC(Oc2ccc(Br)cn2)CC1] | Intermediate 43 |
| 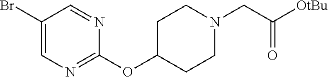 [SMILES: CC(C)(C)OC(=O)CN1CCC(Oc2ncc(Br)cn2)CC1] | Intermediate 44 |
| 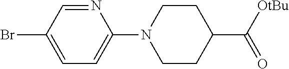 [SMILES: CC(C)(C)OC(=O)C1CCN(c2ccc(Br)cn2)CC1] | Intermediate 45 |
| 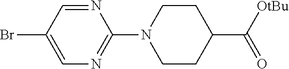 [SMILES: CC(C)(C)OC(=O)C1CCN(c2ncc(Br)cn2)CC1] | Intermediate 46 |
| 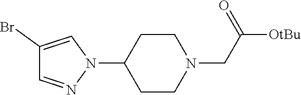 [SMILES: CC(C)(C)OC(=O)CN1CCC(n2cc(Br)cn2)CC1] | Intermediate 47 |
| 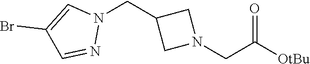 [SMILES: CC(C)(C)OC(=O)CN1CC(Cn2cc(Br)cn2)C1] | Intermediate 48 |
| 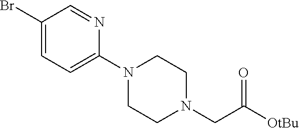 [SMILES: CC(C)(C)OC(=O)CN1CCN(c2ccc(Br)cn2)CC1] | Intermediate 49 |
| 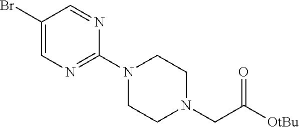 [SMILES: CC(C)(C)OC(=O)CN1CCN(c2ncc(Br)cn2)CC1] | Intermediate 50 |
| 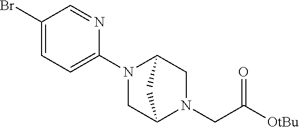 [SMILES: CC(C)(C)OC(=O)CN1C[C@H]2C[C@@H]1CN2c1ccc(Br)cn1] | Intermediate 51 |
| 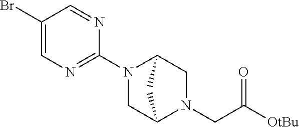 [SMILES: CC(C)(C)OC(=O)CN1C[C@H]2C[C@@H]1CN2c1ncc(Br)cn1] | Intermediate 52 |
| 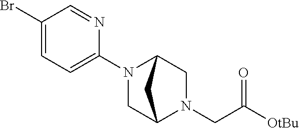 [SMILES: CC(C)(C)OC(=O)CN1C[C@@H]2C[C@H]1CN2c1ccc(Br)cn1] | Intermediate 53 |
| 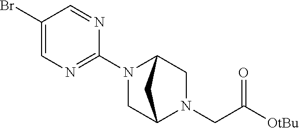 [SMILES: CC(C)(C)OC(=O)CN1C[C@@H]2C[C@H]1CN2c1ncc(Br)cn1] | Intermediate 54 |
| 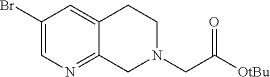 [SMILES: CC(C)(C)OC(=O)CN1CCc2cc(Br)cnc2C1] | Intermediate 55 |
| 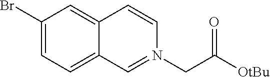 | Intermediate 56 |
| 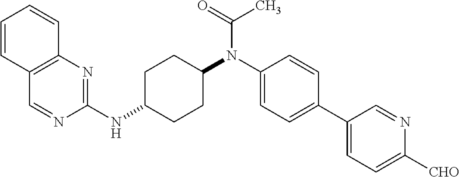 | Intermediate 57 |
| 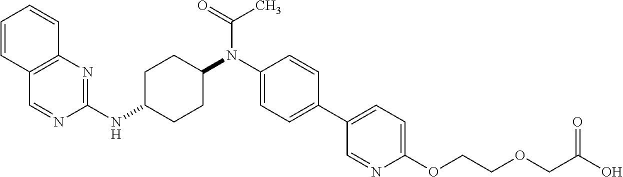 | Intermediate 58 |
| 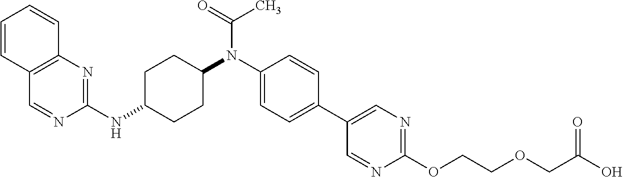 | Intermediate 59 |
| 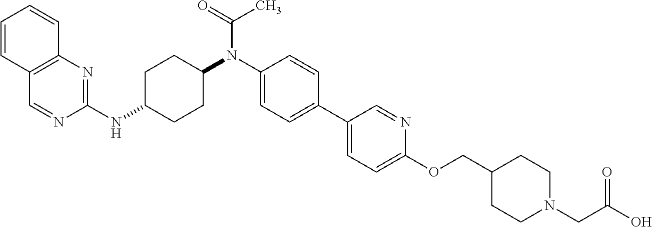 | Intermediate 60 |
| 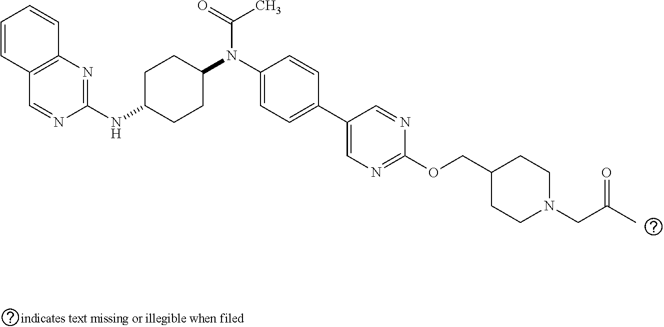 | Intermediate 61 |
| 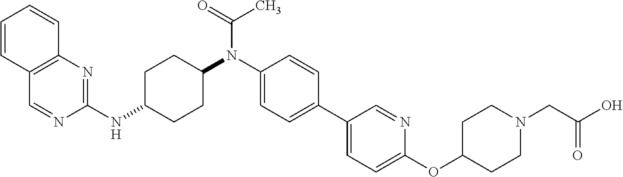 | Intermediate 62 |
| 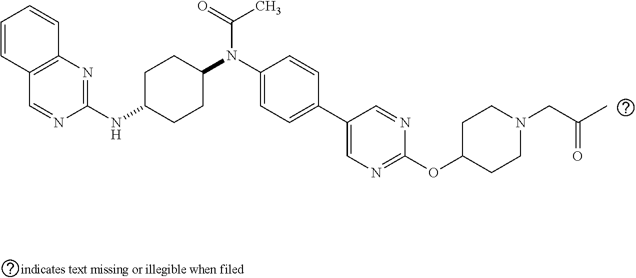 | Intermediate 63 |
| 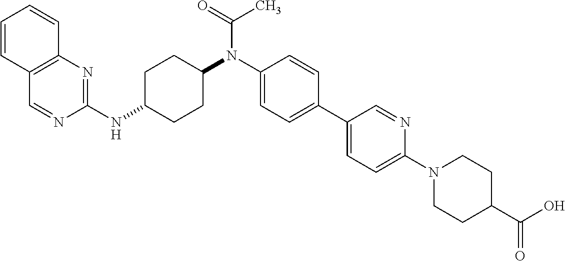 | Intermediate 64 |
| 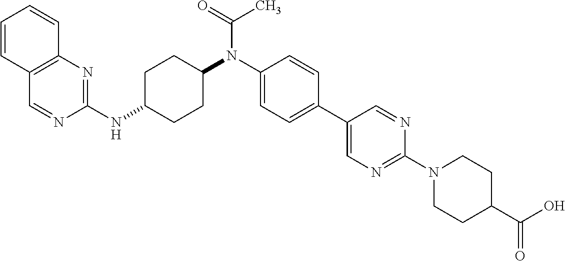 | Intermediate 65 |
| 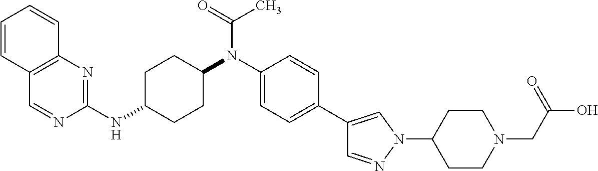 | Intermediate 66 |
| 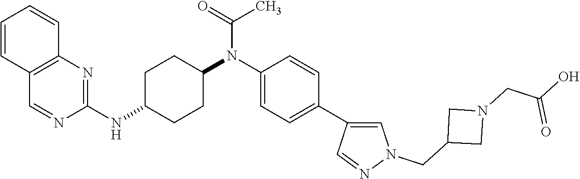 | Intermediate 67 |
| 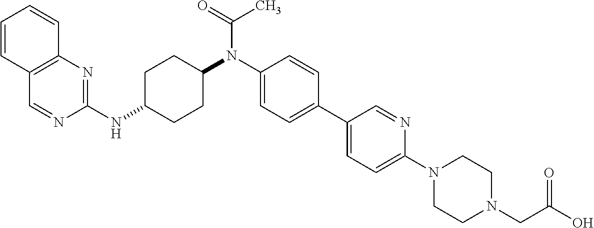 | Intermediate 68 |
| 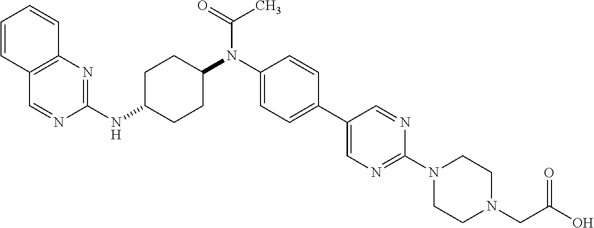 | Intermediate 69 |
| 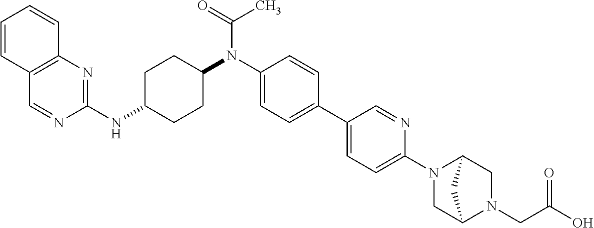 | Intermediate 70 |
| 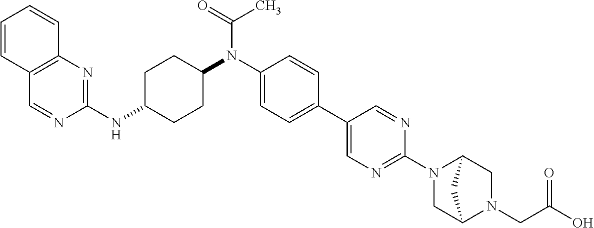 | Intermediate 71 |
| 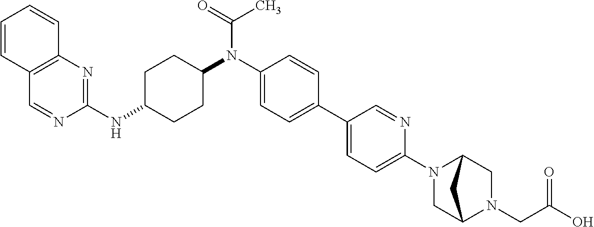 | Intermediate 72 |
| 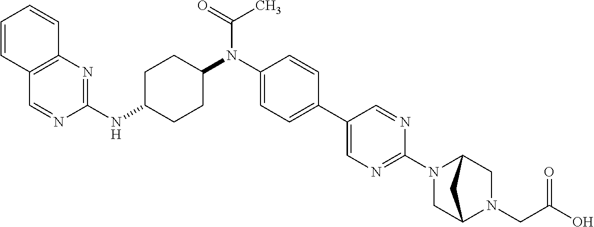 | Intermediate 73 |
| 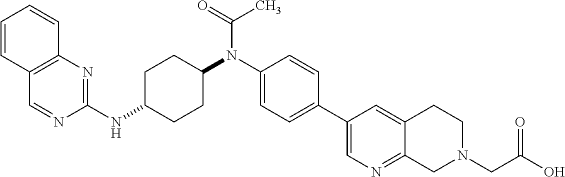 | Intermediate 74 |
| 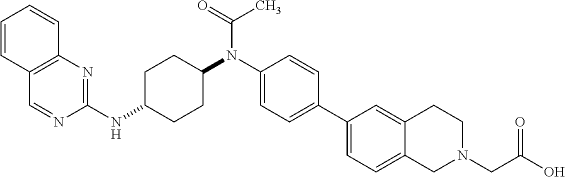 | Intermediate 75 |

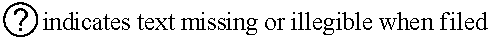

### Intermediate 1: N-(4-bromophenyl)-N-((1R,4R)-4-(quinazolin-2-ylamino)cyclohexyl) acetamide

### Synthesis Step 1: tert-butyl ((1R,4R)-4-((4-bromophenyl)amino)cyclohexyl) carbamate

In a 250 mL round-bottom flask, add 7.9 g (28.1 mmol) of 1-bromo-4-iodobenzene, 5.0 g (23.4 mmol) of trans-(4-aminocyclohexyl) carbamic acid tert-butyl ester, 3.4 g (35.0 mmol) of sodium tert-butoxide, and 1.3 g (2.3 mmol) of Xantphos (4,5-bis(diphenylphosphino)-9,9-dimethylxanthene), and dissolve in 40 mL of toluene solvent. After purging with nitrogen three times, add 856 mg (0.93 mmol) of Pd2(dba)3. Purge with nitrogen three more times, heat to 70 °C, and stir for 2 hours.

After TLC shows the complete consumption of the starting material, filter with diatomaceous earth. Wash the filter cake with ethyl acetate. Concentrate the filtrate under reduced pressure and rotary dry. Purify by silica gel column chromatography to obtain 6.5 g, with a yield of 76%. ESI-MS: m/z=369.2 [M+H]⁺

¹H NMR (400 MHz, DMSO-d6) δ 7.15 (d, J=8.8 Hz, 2H), 6.79 (d, J=7.9 Hz, 1H), 6.49 (d, J=8.9 Hz, 2H), 5.62 (d, J=8.0 Hz, 1H), 3.25-3.15 (m, 1H), 3.13-2.99 (m, 1H), 1.93 (d, J=11.5 Hz, 2H), 1.78 (d, J=11.0 Hz, 2H), 1.37 (s, 9H), 1.30-1.19 (m, 2H), 1.18-1.07 (m, 2H).

### Synthesis Step 2: N-((1R,4R)-4-aminocyclohexyl)-N-(4-bromophenyl)acetamide hydrochloride

In a 250 mL round-bottom flask, add 6.5 μg (17.68 mmol) of tert-butyl ((1r,4r)-4-((4-bromophenyl)amino)cyclohexyl)carbamate, and dissolve in 30 mL of anhydrous tetrahydrofuran. Add 16 mL (88.40 mmol) of anhydrous triethylamine. After purging with nitrogen three times, stir for 5 minutes in an ice bath. Slowly add 4.2 μg (0.93 mmol) of acetyl chloride dropwise to the reaction mixture. Continue stirring in an ice bath for 3 hours.

After TLC shows the complete consumption of the starting material, wash the reaction mixture with ethyl acetate. Concentrate the filtrate under reduced pressure and rotary dry. The resulting solid is slurried, and after the filter cake is washed with 50% EA/PE, it is vacuum dried.

Treat with a 2N hydrogen chloride-ethyl acetate solution. After TLC shows the complete consumption of the starting material, distill the reaction solvent under reduced pressure to obtain 4.3 g of product, with a yield of 71%. ESI-MS: m/z=311.2 [M+H]⁺.

### Synthesis Step 3: N-(4-bromophenyl)-N-((1R,4R)-4-(quinazolin-2-ylamino)cyclohexyl)acetamide

In a 100 mL round-bottom flask, add 1.9 g (5.66 mmol) of N-((1R,4R)-4-aminocyclohexyl)-N-(4-bromophenyl)acetamide hydrochloride and 1.3 g (7.91 mmol) of 2-chloroquinazoline, dissolved in anhydrous DMF solution. Cool the reaction system to 0 °C, then slowly add 5.1 g (33.96 mmol) of DBU (1,8-diazabicyclo[5.4.0]undec-7-ene) dropwise to the reaction mixture. Afterward, heat to 120 °C and stir for 8 hours.

After TLC shows the complete consumption of the starting material, pour the reaction mixture into 100 ml of water and extract three times with 60 ml of dichloromethane. Concentrate the organic phase under reduced pressure. Dissolve the resulting solid in a minimum amount of ethyl acetate, let it stand at −20 °C for 7 hours for recrystallization, and filter with suction while cold. Wash the filter cake with 50% ethyl acetate/petroleum ether. Vacuum dry the obtained solid filter cake to get 1.4 g of product, with a yield of 56%. ESI-MS: m/z=439.36 [M+H]+.

### Intermediate 2: N-((1R,4R)-4-(quinazolin-2-ylamino)cyclohexyl)-N-(4-(4,4,5,5-tetramethyl-1,3,2-dioxaborolan-2-yl)phenyl)acetamide

In a 100 mL round-bottom flask, add 1.0 g (2.27 mmol) of intermediate 1, 864.9 mg (3.41 mmol) of bis(pinacolato)diboron, and 889.8 mg (9.08 mmol) of potassium acetate, dissolved in anhydrous dioxane solution. After purging with nitrogen three times, add 331.9 mg (0.45 mmol) of Pd(dppf)₂Cl₂. Purge with nitrogen three more times, heat to 65 °C, and stir for 2 hours.

After TLC shows the complete consumption of the starting material, wash twice with saturated saline and separate the organic phase. Concentrate the organic phase under reduced pressure and rotary dry. Purify by silica gel column chromatography to obtain 742 mg of intermediate 2, with a yield of 67%. ESI-MS: m/z=487.39 [M+H]⁺.

### Intermediate 3: tert-butyl 5-(5-bromopyridin-2-yl)hexahydropyrrolo[3,4-c]pyrrole-2(1H)-carboxylate

In a 50 mL round-bottom flask, add 498.1 mg (2.83 mmol) of 2-fluoro-5-bromopyridine, 500.0 mg (2.36 mmol) of tert-butyl hexahydropyrrolo[3,4-c]pyrrole-2(1H)-carboxylate, and 1.27 mL (7.08 mmol) of N,N-diisopropylethylamine. Dissolve in anhydrous DMSO solution. After purging with nitrogen, heat to 120 °C and stir for 2 hours.

After TLC shows the complete consumption of the starting material, wash twice with saturated saline and separate the organic phase. Concentrate the organic phase under reduced pressure and rotary dry. Purify by silica gel column chromatography to obtain 742 mg of intermediate 3, with a yield of 71%. ESI-MS: m/z=368.13 [M+H]⁺.

### Intermediate 4: tert-butyl 3-((5-bromopyrimidin-2-yl)oxy)azetidine-1-carboxylate

Synthesis Step: In a 50 mL round-bottom flask, add 500 mg (2.86 mmol) of N-Boc-3-hydroxyazetidine, dissolved in anhydrous THF solution. In an ice bath, add 171.6 mg (4.29 mmol) of sodium hydride. After stirring at 0 °C for 15 minutes, add 665.8 mg (3.43 mmol) of 5-bromo-2-chloropyrimidine. Warm to room temperature and stir for 4 hours.

After TLC shows the complete consumption of the starting material, wash twice with saturated saline and separate the organic phase. Concentrate the organic phase under reduced pressure and rotary dry. Purify by silica gel column chromatography to obtain 805 mg of intermediate 4, with a yield of 85%. ESI-MS: m/z=330.09 [M+H]⁺.
¹H-NMR (400 MHz, Chloroform-d) δ 8.52 (s, 2H), 5.26 (tt, J=6.6, 4.3 Hz, 1H), 4.30 (ddd, J=10.0, 6.6, 1.2 Hz, 2H), 4.02 (ddd, J=10.0, 4.3, 1.1 Hz, 2H), 1.43 (s, 9H).

### Intermediate 5: tert-butyl (S)-3-((5-bromopyrimidin-2-yl)oxy)pyrrolidine-1-carboxylate

Synthesis Step: Following the method for Intermediate 4, replace the N-Boc-3-hydroxyazetidine in the synthesis step with (S)-1-N-tert-butoxycarbonyl-3-hydroxypyrrolidine to obtain Intermediate 5. ESI-MS: m/z=344.12 [M+H]⁺.

### Intermediate 6: tert-butyl (R)-3-((5-bromopyrimidin-2-yl)oxy)pyrrolidine-1-carboxylate

Synthesis Step: Following the method for Intermediate 4, replace the N-Boc-3-hydroxyazetidine in the synthesis step with (R)-1-N-tert-butoxycarbonyl-3-hydroxypyrrolidine to obtain Intermediate 6. ESI-MS: m/z=344.12 [M+H]⁺.

### Intermediate 7: tert-butyl 4-((4-bromo-2-oxopyridin-1(2H)-yl)methyl)piperidine-1-carboxylate

Synthesis Step: In a 50 mL round-bottom flask, add 315 mg (1.81 mmol) of 4-bromopyridin-2(1H)-one and 500 mg (1.81 mmol) of tert-butyl 4-(bromomethyl)piperidine-1-carboxylate, dissolved in anhydrous acetonitrile solution. Add 1.17 g (3.62 mmol) of cesium chloride and stir at room temperature for 6 hours. After TLC shows the complete consumption of the starting material, wash twice with saturated saline and separate the organic phase. Concentrate the organic phase under reduced pressure and rotary dry. Purify by silica gel column chromatography to obtain 364 mg of intermediate 7, with a yield of 55%. ESI-MS: m/z=371.18 [M+H]⁺. ¹H NMR (400 MHz, Chloroform-d) δ 7.03 (d, J=7.2 Hz, 1H), 6.81 (d, J=2.2 Hz, 1H), 6.30 (dd, J=7.2, 2.1 Hz, 1H), 4.11 (s, 2H), 3.74 (s, 2H), 2.64 (t, J=13.0 Hz, 2H), 2.02 (dtq, J=11.3, 7.4, 3.6 Hz, 1H), 1.75 (s, 2H), 1.60 (d, J=13.1 Hz, 2H), 1.43 (s, 9H).

### Intermediate 8: tert-butyl 3-((4-bromo-2-oxopyridin-1(2H)-yl)methyl)azetidine-1-carboxylate

Synthesis Step: Following the method for Intermediate 7, replace the tert-butyl 4-(bromomethyl)piperidine-1-carboxylate in the synthesis step with tert-butyl 3-(bromomethyl)azetidine-1-carboxylate to obtain Intermediate 8. ESI-MS: m/z=343.11 [M+H]⁺.

### Intermediate 9: tert-butyl 3-((4-bromo-2-oxopyridin-1(2H)-yl)methyl)pyrrolidine-1-carboxylate

Synthesis Method: Following the method for Intermediate 7, replace the tert-butyl 4-(bromomethyl)piperidine-1-carboxylate in the synthesis step with 1-Boc-3-(bromomethyl)pyrrolidine to obtain Intermediate 9. ESI-MS: m/z=357.17 [M+H]⁺.

### Intermediate 10: 2-(2,6-dioxopiperidin-3-yl)-5-(piperazin-1-yl)isoindoline-1,3-dione

### Synthesis Step 1: tert-butyl 4-(2-(2,6-dioxopiperidin-3-yl)-1,3-dioxoisoindolin-5-yl)piperazine-1-carboxylate

In a 50 mL round-bottom flask, add 400.0 mg (1.45 mmol) of 2-(2,6-dioxopiperidin-3-yl)-5-fluoroisoindoline-1,3-dione, 403.6 mg (2.17 mmol) of tert-butyl piperazine-1-carboxylate, and 610 μL (4.35 mmol) of anhydrous triethylamine, dissolved in anhydrous DMSO solution. Heat to 100 °C and stir for 12 hours. After TLC shows the complete consumption of the starting material, wash twice with saturated saline and separate the organic phase. Concentrate the organic phase under reduced pressure and rotary dry. Purify by silica gel column chromatography to obtain 511 mg, with a yield of 80%. ESI-MS: m/z=443.22 [M+H]⁺.

### Synthesis Step 2: 2-(2,6-dioxopiperidin-3-yl)-5-(piperazin-1-yl)isoindoline-1,3-dione

In a 25 mL round-bottom flask, add 511.0 mg (1.16 mmol) of tert-butyl 4-(2-(2,6-dioxopiperidin-3-yl)-1,3-dioxoisoindolin-5-yl)piperazine-1-carboxylate and treat with a 2N hydrogen chloride-ethyl acetate solution. After TLC shows the complete consumption of the starting material, extract twice with saturated aqueous sodium bicarbonate solution and dichloromethane. Concentrate the organic phase under reduced pressure, rotary dry, and vacuum dry to obtain 345 mg of intermediate 10, with a yield of 87%. ESI-MS: m/z=343.23 [M+H]⁺.

### Intermediate 11: 3-(2-(2,6-dioxopiperidin-3-yl)-1,3-dioxoisoindolin-5-yl)acrylate

### Synthesis Step 1: tert-butyl 3-(2-(2,6-dioxopiperidin-3-yl)-1,3-dioxoisoindolin-5-yl)acrylate

In a 50 mL round-bottom flask, add 400.0 mg (1.19 mmol) of 5-bromo-2-(2,6-dioxopiperidin-3-yl)isoindoline-1,3-dione, 304.6 mg (2.38 mmol) of tert-butyl acrylate, 456.9 mg (2.38 mmol) of DIPEA, and 26 mg (0.1 mmol) of triphenylphosphine, dissolved in anhydrous DMF solution. After purging with nitrogen three times, add 13.4 mg (0.06 mmol) of palladium acetate. Purge with nitrogen three more times, heat to 100 °C, and stir for 9 hours. After TLC shows the complete consumption of the starting material, wash twice with saturated saline and separate the organic phase. Concentrate the organic phase under reduced pressure and rotary dry. Purify by silica gel column chromatography to obtain 351 mg, with a yield of 77%. ESI-MS: m/z=385.13 [M+H]⁺. ¹H NMR (400 MHz, DMSO-d6) δ=11.21 (s, 1H), 8.34 (s, 1H), 8.22 (d, J=7.8 Hz, 1H), 7.98 (d, J=7.7 Hz, 1H), 7.71 (d, J=16.1 Hz, 1H), 6.82 (d, J=16.1 Hz, 1H), 5.23 (dd, J=12.1, 5.8 Hz, 1H), 2.96-2.84 (m, 1H), 2.62-2.51 (m, 2H), 2.12-1.97 (m, 1H), 1.44 (s, 9H).

### Synthesis Step 2: 3-(2-(2,6-dioxopiperidin-3-yl)-1,3-dioxoisoindolin-5-yl)acrylate

In a 25 mL round-bottom flask, add 350 mg (0.91 mmol) of tert-butyl 3-(2-(2,6-dioxopiperidin-3-yl)-1,3-dioxoisoindolin-5-yl)acrylate and treat with a 50% trifluoroacetic acid solution. After TLC shows the complete consumption of the starting material, concentrate under reduced pressure, rotary dry, and vacuum dry to obtain 276 mg of intermediate 11, with a yield of 92%. ESI-MS: m/z=329.14 [M+H]⁺.

### Intermediate 12: tert-butyl 3-(2-(2,6-dioxopiperidin-3-yl)-1,3-dioxoisoindolin-5-yl)acrylate

Synthesis Method: Following the method for Intermediate 10, replace the tert-butyl piperazine-1-carboxylate in the synthesis step with tert-butyl glycinate to obtain Intermediate 12. ESI-MS: m/z=332.06 [M+H]⁺.

### Intermediate 13: N-(4-(piperazin-1-yl)phenyl)-N-((1R,4R)-4-(quinazolin-2-ylamino)cyclohexyl)acetamide

### Synthesis Step 1: tert-butyl 4-(4-(N-((1R,4R)-4-(quinazolin-2-ylamino)cyclohexyl)acetamido)phenyl)piperazine-1-carboxylate

In a 50 mL round-bottom flask, add 300.0 mg (0.68 mmol) of intermediate 1, 191.6 mg (1.03 mmol) of tert-butyl piperazine-1-carboxylate, 195.8 mg (2.04 mmol) of sodium tert-butoxide, and 39.4 mg (0.07 mmol) of Xantphos, dissolved in 10 mL of toluene solvent. After purging with nitrogen three times, add 37.6 mg (0.04 mmol) of Pd₂(dba)₃. Purge with nitrogen three more times, heat to 80 °C, and stir for 2 hours. After TLC shows the complete consumption of the starting material, concentrate the filtrate under reduced pressure and rotary dry. Purify by silica gel column chromatography to obtain 342 mg of product, with a yield of 91%. ESI-MS: m/z=545.68 [M+H]⁺.

### Synthesis Step 2: 2-(2,6-dioxopiperidin-3-yl)-5-(piperazin-1-yl)isoindoline-1,3-dione

In a 25 mL round-bottom flask, add 340.0 mg (0.62 mmol) of tert-butyl 4-(4-(N-((1R,4R)-4-(quinazolin-2-ylamino)cyclohexyl)acetamido)phenyl)piperazine-1-carboxylate and treat with a 2N hydrogen chloride-ethyl acetate solution. After TLC shows the complete consumption of the starting material, extract twice with saturated aqueous sodium bicarbonate solution and dichloromethane. Concentrate the organic phase under reduced pressure, rotary dry, and vacuum dry to obtain 268 mg of intermediate 13, with a yield of 97%. ESI-MS: m/z=445.15 [M+H]⁺.

### Intermediate 14: N-(4-(6-(piperazin-1-yl)pyridin-3-yl)phenyl)-N-((1R,4R)-4-(quinazolin-2-ylamino)cyclohexyl)acetamide

### Synthesis Step 1: tert-butyl 4-(5-(4-(N-((1R,4R)-4-(quinazolin-2-ylamino)cyclohexyl)acetamido)phenyl)pyridin-2-yl)piperazine-1-carboxylate

In a 25 ml round-bottom flask, add 80.0 mg (0.16 mmol) of intermediate 2, 340.0 mg (0.25 mmol) of 4-Boc-1-(5-bromo-2-pyridyl)piperazine, and 85.6 mg (0.48 mmol) of cesium carbonate, dissolved in a mixed solvent of toluene:ethanol:water=4:4:1. After purging with nitrogen three times, add 18.5 mg (0.16 mmol) of Pd(PPh₃)₄. Purge with nitrogen three more times, heat to 100 °C, and stir for 3 hours. After TLC shows the complete consumption of the starting material, extract three times with 20 ml of dichloromethane. Concentrate the organic phase under reduced pressure and rotary dry. Purify by silica gel column chromatography to obtain 65 mg, with a yield of 78%. ESI-MS: m/z=622.34 [M+H]⁺.

### Synthesis Step 2: N-(4-(6-(piperazin-1-yl)pyridin-3-yl)phenyl)-N-((1R,4R)-4-(quinazolin-2-ylamino)cyclohexyl)acetamide

In a 25 mL round-bottom flask, add 65.0 mg (0.12 mmol) of tert-butyl 4-(5-(4-(N-((1R,4R)-4-(quinazolin-2-ylamino)cyclohexyl)acetamido)phenyl)pyridin-2-yl)piperazine-1-carboxylate and treat with a 2N hydrogen chloride-ethyl acetate solution. After TLC shows the complete consumption of the starting material, extract twice with saturated aqueous sodium bicarbonate solution and dichloromethane. Concentrate the organic phase under reduced pressure, rotary dry, and vacuum dry to obtain 52 mg of intermediate 14, with a yield of 98%. ESI-MS: m/z=522.31 [M+H]⁺.

### Intermediate 15: N-(4-(6-(hexahydropyrrolo[3,4-c]pyrrol-2(1H)-yl)pyridin-3-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with Intermediate 3 to obtain Intermediate 15. ESI-MS: m/z=548.42 [M+H]⁺.

### Intermediate 16: N-(4-(6-(hexahydropyrrolo[3,4-c]pyrrol-2(1H)-yl)pyridin-3-yl)phenyl)-N-((1R,4R)-4-(quinazolin-2-ylamino)cyclohexyl)acetamide

Synthesis Method: Following the method for Intermediate 13, replace the tert-butyl piperazine-1-carboxylate in the synthesis step with tert-butyl 4-(piperazin-1-ylmethyl)piperidine-1-carboxylate to obtain Intermediate 16. ESI-MS: m/z=542.45 [M+H]⁺.

### Intermediate 17: N-(4-(2-(piperazin-1-yl)pyrimidin-5-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with tert-butyl 4-(5-bromopyrimidin-2-yl)piperazine-1-carboxylate to obtain Intermediate 17. ESI-MS: m/z=523.38 [M+H]⁺.

### Intermediate 18: N-(4-(2-(azetidin-3-yloxy)pyrimidin-5-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with Intermediate 4 to obtain Intermediate 18. ESI-MS: m/z=510.67 [M+H]⁺.

### Intermediate 19: N-(4-(2-(((R)-pyrrolidin-3-yl)oxy)pyrimidin-5-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with Intermediate 6 to obtain Intermediate 19. ESI-MS: m/z=524.13 [M+H]⁺.

### Intermediate 20: N-(4-(2-(((S)-pyrrolidin-3-yl)oxy)pyrimidin-5-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with Intermediate 5 to obtain Intermediate 20. ESI-MS: m/z=524.11 [M+H]⁺.

### Intermediate 21: N-(4-(2-oxo-1-(piperidin-4-ylmethyl)-1,2-dihydropyridin-4-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with Intermediate 7 to obtain Intermediate 21. ESI-MS: m/z=551.84 [M+H]⁺.

### Intermediate 22: N-(4-(1-(azetidin-3-ylmethyl)-2-oxo-1,2-dihydropyridin-4-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with Intermediate 8 to obtain Intermediate 22. ESI-MS: m/z=523.72 [M+H]⁺.

### Intermediate 23: N-(4-(1-(azetidin-3-yl)-1H-pyrazol-4-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with tert-butyl 3-(4-bromo-1H-pyrazol-1-yl)azetidine-1-carboxylate to obtain Intermediate 23. ESI-MS: m/z=482.46 [M+H]⁺.

### Intermediate 24: N-(4-(1-(azetidin-3-ylmethyl)-1H-pyrazol-4-yl)phenyl)-N-((1R,4R)-4-(quinazolin-2-ylamino)cyclohexyl)acetamide

Synthesis Method: Following the method for Intermediate 14, replace the tert-butyl 4-(5-bromo-2-pyridyl)piperazine in the synthesis step with tert-butyl 3-((4-bromo-1H-pyrazol-1-yl)methyl)azetidine-1-carboxylate to obtain Intermediate 24. ESI-MS: m/z=496.46 [M+H]⁺.

### Intermediate 25: N-(4-(1-(pyrrolidin-3-yl)-1H-pyrazol-4-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with tert-butyl 3-(4-bromo-1H-pyrazol-1-yl)pyrrolidine-1-carboxylate to obtain Intermediate 25. ESI-MS: m/z=496.61 [M+H]⁺.

### Intermediate 26: N-(4-(1-(piperidin-4-yl)-1H-pyrazol-4-yl)phenyl)-N-((1R,4R)-4-(quinazolin-2-ylamino)cyclohexyl)acetamide

Synthesis Method: Following the method for Intermediate 14, replace the 4-Boc-1-(5-bromo-2-pyridyl)piperazine in the synthesis step with tert-butyl 4-(4-bromopyrazol-1-yl)piperidine-1-carboxylate to obtain Intermediate 26. ESI-MS: m/z=510.35 [M+H]⁺.

### Intermediate 27: N-(4-(2,8-diazaspiro[4.5]decan-8-yl)phenyl)-N-((1R,4R)-4-(quinazolin-2-ylamino)cyclohexyl)acetamide

Synthesis Method: Following the method for Intermediate 13, replace the tert-butyl piperazine-1-carboxylate in the synthesis step with tert-butyl 2,8-diazaspiro[4.5]decane-2-carboxylate to obtain Intermediate 27. ESI-MS: m/z=499.62 [M+H]⁺.

### Intermediate 28: N-(4-(3,9-diazaspiro[5.5]undecan-3-yl)phenyl)-N-((1r,4r)-4-(quinazolin-2-ylamino)cyclohexyl)acetamide

Synthesis Method: Following the method for Intermediate 13, replace the tert-butyl piperazine-1-carboxylate in the synthesis step with tert-butyl 3,9-diazaspiro[5.5]undecane-3-carboxylate to obtain Intermediate 28. ESI-MS: m/z=513.67 [M+H]⁺.

### Intermediate 29: N-(4-(2,7-diazaspiro[3.5]non-7-yl)phenyl)-N-((1R,4R)-4-(quinazolin-2-ylamino)cyclohexyl)acetamide

Synthesis Method: Following the method for Intermediate 13, replace the tert-butyl piperazine-1-carboxylate in the synthesis step with tert-butyl 2,7-diazaspiro[3.5]nonane-2-carboxylate to obtain Intermediate 29. ESI-MS: m/z=485.60 [M+H]⁺.

### Intermediate 30: N-(4-(4-(prop-2-yn-1-yl)piperazin-1-yl)phenyl)-N-((1R,4R)-4-(quinazolin-2-ylamino)cyclohexyl)acetamide

Synthesis Method: Following synthesis step 1 of Intermediate 13, replace the tert-butyl piperazine-1-carboxylate in the synthesis step with 1-(prop-2-yn-1-yl)piperazine to obtain Intermediate 30. ESI-MS: m/z=483.44 [M+H]⁺.

### Intermediate 31: N-(4-(4-(piperidin-4-yl)piperazin-1-yl)phenyl)-N-((1R,4R)-4-(quinazolin-2-ylamino)cyclohexyl)acetamide

Synthesis Method: Following the method for Intermediate 13, replace the tert-butyl piperazine-1-carboxylate in the synthesis step with tert-butyl 4-(piperazin-1-yl)piperidine-1-carboxylate to obtain Intermediate 31. ESI-MS: m/z=528.41 [M+H]⁺.

### Intermediate 32: N-(4-(4-(azetidin-3-yl)piperazin-1-yl)phenyl)-N-((1R,4R)-4-(quinazolin-2-ylamino)cyclohexyl)acetamide

Synthesis Method: Following the method for Intermediate 13, replace the tert-butyl piperazine-1-carboxylate in the synthesis step with tert-butyl 3-(piperazin-1-yl)azetidine-1-carboxylate to obtain Intermediate 32. ESI-MS: m/z=500.18 [M+H]⁺.

### Intermediate 33: N-(4-(4-(pyrrolidin-3-yl)piperazin-1-yl)phenyl)-N-((1R,4R)-4-(quinazolin-2-ylamino)cyclohexyl)acetamide

Synthesis Method: Following the method for Intermediate 13, replace the tert-butyl piperazine-1-carboxylate in the synthesis step with tert-butyl 3-(piperazin-1-yl)pyrrolidine-1-carboxylate to obtain Intermediate 33. ESI-MS: m/z=514.20 [M+H]⁺.

### Intermediate 34: N-(4-(4-(pyrrolidin-3-ylmethyl)piperazin-1-yl)phenyl)-N-((1R,4R)-4-(quinazolin-2-ylamino)cyclohexyl)acetamide

### Synthesis Step 1: tert-butyl 3-((4-(4-(N-((1R,4R)-4-(quinazolin-2-ylamino)cyclohexyl)acetamido)phenyl)piperazin-1-yl)methyl)pyrrolidine-1-carboxylate

In a 25 ml round-bottom flask, add 85.0 mg (0.19 mmol) of Intermediate 13 and 76.0 mg (0.38 mmol) of 1-Boc-3-pyrrolidinecarboxaldehyde, dissolved in dichloromethane. Add a catalytic amount of acetic acid and stir at room temperature for 1 hour. Then add 120.8 mg (0.57 mmol) of sodium triacetoxyborohydride and stir at room temperature for 6 hours. After TLC shows the complete consumption of the starting material, extract three times with 20 ml of dichloromethane. Concentrate the organic phase under reduced pressure and rotary dry. Purify by silica gel column chromatography to obtain 100 mg, with a yield of 84%. ESI-MS: m/z=628.39 [M+H]⁺.

### Synthesis Step 2: N-(4-(4-(pyrrolidin-3-ylmethyl)piperazin-1-yl)phenyl)-N-((1R,4R)-4-(quinazolin-2-ylamino)cyclohexyl)acetamide

In a 25 mL round-bottom flask, add 100 mg (0.16 mmol) of tert-butyl 3-((4-(4-(N-((1R,4R)-4-(quinazolin-2-ylamino)cyclohexyl)acetamido)phenyl)piperazin-1-yl)methyl)pyrrolidine-1-carboxylate and treat with a 2N hydrogen chloride-ethyl acetate solution. After TLC shows the complete consumption of the starting material, extract twice with saturated aqueous sodium bicarbonate solution and dichloromethane. Concentrate the organic phase under reduced pressure, rotary dry, and vacuum dry to obtain 82 mg of intermediate 34, with a yield of 98%. ESI-MS: m/z=528.37[M+H]⁺.

### Intermediate 35: N-(4-(4-(azetidin-3-ylmethyl)piperazin-1-yl)phenyl)-N-((1R,4R)-4-(quinazolin-2-ylamino)cyclohexyl)acetamide

Synthesis Method: Following the method for Intermediate 34, replace the 1-Boc-3-pyrrolidinecarboxaldehyde in the synthesis step with tert-butyl 3-formylazetidine-1-carboxylate to obtain Intermediate 35. ESI-MS: m/z=514.36 [M+H]⁺.

### Intermediate 36: 5-(4-(N-((1R,4R)-4-(quinazolin-2-ylamino)cyclohexyl)acetamido)phenyl)nicotinic acid

Synthesis Method: Following the method for Intermediate 14, replace the 4-Boc-1-(5-bromo-2-pyridyl)piperazine in the synthesis step with tert-butyl 5-bromonicotinate and replace the 2N hydrogen chloride-ethyl acetate solution with a 50% trifluoroacetic acid solution to obtain Intermediate 36. ESI-MS: m/z=482.09 [M+H]⁺.

### Intermediate 37: 5-(4-(N-((1R,4R)-4-(quinazolin-2-ylamino)cyclohexyl)acetamido)phenyl)picolinic acid

Synthesis Method: Following the method for Intermediate 14, replace the 4-Boc-1-(5-bromo-2-pyridyl)piperazine in the synthesis step with tert-butyl 5-bromopyridine-2-carboxylate and replace the 2N hydrogen chloride-ethyl acetate solution with a 50% trifluoroacetic acid solution to obtain Intermediate 37. ESI-MS: m/z=482.09 [M+H]⁺.

### Intermediate 38: N-(4-(2-(4-(5-amino-2-fluorophenyl)piperazin-1-yl)pyrimidin-5-yl)phenyl)-N-((1R,4R)-4-(quinazolin-2-ylamino)cyclohexyl)acetamide

### Synthesis Step 1: N-(4-(2-(4-(2-fluoro-5-nitrophenyl)piperazin-1-yl)pyrimidin-5-yl)phenyl)-N-((1R,4R)-4-(quinazolin-2-ylamino)cyclohexyl)acetamide

In a 25 ml round-bottom flask, add 100.0 mg (0.19 mmol) of Intermediate 17 and 60.7 mg (0.38 mmol) of 3,4-difluoronitrobenzene, dissolved in dimethyl sulfoxide. While stirring, add 205 μL (1.15 mmol) of N,N-diisopropylethylamine dropwise. Heat to 100 °C and stir for 2 hours. After TLC shows the complete consumption of the starting material, wash with water and extract three times with 20 ml of ethyl acetate. Concentrate the organic phase under reduced pressure and rotary dry. Purify by silica gel column chromatography to obtain 104 mg, with a yield of 84%. ESI-MS: m/z=662.51 [M+H]⁺.

### Synthesis Step 2: N-(4-(2-(4-(5-amino-2-fluorophenyl)piperazin-1-yl)pyrimidin-5-yl)phenyl)-N-((1R,4R)-4-(quinazolin-2-ylamino)cyclohexyl)acetamide

In a 25 mL round-bottom flask, add 104 mg (0.16 mmol) of N-(4-(2-(4-(2-fluoro-5-nitrophenyl)piperazin-1-yl)pyrimidin-5-yl)phenyl)-N-((1R,4R)-4-(quinazolin-2-ylamino)cyclohexyl)acetamide, dissolved in a 3:1 ethanol:water solution. Add 87.9 mg (1.57 mmol) of iron powder and 84.0 mg (1.57 mmol) of ammonium chloride. Heat to 70 °C and stir for 6 hours. After TLC shows the complete consumption of the starting material, filter. Extract the filtrate three times with 20 ml of dichloromethane. Concentrate the organic phase under reduced pressure, rotary dry, and vacuum dry to obtain 83 mg of intermediate 38, with a yield of 81%. ESI-MS: m/z=632.45[M+H]⁺.

### Intermediate 39: tert-butyl 2-(2-((5-bromopyridin-2-yl)oxy)ethoxy)acetate

In a 100 ml round-bottom flask, add 430.0 mg (2.44 mmol) of tert-butyl 2-(2-hydroxyethoxy)acetate, 860.0 mg (4.88 mmol) of 2-fluoro-5-bromopyridine, and 2.4 g (7.32 mmol) of cesium carbonate, and dissolve in anhydrous dimethyl sulfoxide. Heat the reaction system to 100 °C and stir for 6 hours.

After TLC shows the complete consumption of the starting material, pour the reaction mixture into 100 ml of saturated saline. Extract three times with 60 mL of ethyl acetate. Concentrate the organic phase under reduced pressure and rotary dry. Purify by silica gel column chromatography to obtain 697 mg of intermediate 39, with a yield of 86%. ESI-MS: m/z=332.17[M+H]⁺.

### Intermediate 40: tert-butyl 2-(2-((5-bromopyrimidin-2-yl)oxy)ethoxy)acetate

In a 100 ml round-bottom flask, add 430.0 mg (2.44 mmol) of tert-butyl 2-(2-hydroxyethoxy)acetate, 941.8 mg (4.88 mmol) of 5-bromo-2-chloropyrimidine, and 2.4 g (7.32 mmol) of cesium carbonate, dissolved in anhydrous N,N-dimethylformamide. Heat the reaction system to 120 °C and stir for 4 hours. After TLC shows the complete consumption of the starting material, pour the reaction mixture into 100 ml of saturated saline. Extract three times with 60 mL of ethyl acetate. Concentrate the organic phase under reduced pressure and rotary dry. Purify by silica gel column chromatography to obtain 496 mg of intermediate 40, with a yield of 61%. ESI-MS: m/z=333.19[M+H]⁺.

### Intermediate 41: tert-butyl 2-(4-(((5-bromopyridin-2-yl)oxy)methyl)piperidin-1-yl)acetate

### Synthesis Step 1: tert-butyl 2-(4-(hydroxymethyl)piperidin-1-yl)acetate

In a 100 ml round-bottom flask, add 300.0 mg (2.61 mmol) of 4-piperidinemethanol and 534.1 mg (2.74 mmol) of tert-butyl bromoacetate, dissolved in anhydrous acetonitrile. Add 549 μL (3.92 mmol) of anhydrous triethylamine and stir at room temperature for 4 hours. After TLC shows the complete consumption of the starting material, pour the reaction mixture into 80 ml of saturated saline. Extract three times with 60 mL of dichloromethane. Concentrate the organic phase under reduced pressure and rotary dry. Purify by silica gel column chromatography to obtain 447 mg, with a yield of 75%. ESI-MS: m/z=230.87 [M+H]⁺.

### Synthesis Step 2: tert-butyl 2-(4-(((5-bromopyridin-2-yl)oxy)methyl)piperidin-1-yl)acetate

Following the method for Intermediate 39, replace the tert-butyl 2-(2-hydroxyethoxy)acetate in the synthesis step with tert-butyl 2-(4-(hydroxymethyl)piperidin-1-yl)acetate to obtain Intermediate 41. ESI-MS: m/z=384.99 [M+H]⁺.

### Intermediate 42: tert-butyl 2-(4-(((5-bromopyridin-2-yl)oxy)methyl)piperidin-1-yl)acetate

Following the method for Intermediate 40, replace the tert-butyl 2-(2-hydroxyethoxy)acetate in the synthesis step with tert-butyl 2-(4-(hydroxymethyl)piperidin-1-yl)acetate to obtain Intermediate 42. ESI-MS: m/z=386.03 [M+H]⁺.

### Intermediate 43: tert-butyl 2-(4-((5-bromopyridin-2-yl)oxy)piperidin-1-yl)acetate

Following the method for Intermediate 41, replace the 4-piperidinemethanol in the synthesis step with 4-hydroxypiperidine to obtain Intermediate 43. ESI-MS: m/z=370.96 [M+H]⁺.

### Intermediate 44: tert-butyl 2-(4-((5-bromopyridin-2-yl)oxy)piperidin-1-yl)acetate

Following the method for Intermediate 40, replace the tert-butyl 2-(2-hydroxyethoxy)acetate in the synthesis step with tert-butyl 2-(4-hydroxypiperidin-1-yl)acetate to obtain Intermediate 44. The synthesis method for tert-butyl 2-(4-hydroxypiperidin-1-yl)acetate is by reference to Synthesis Step 1 of Intermediate 41, replacing the 4-piperidinemethanol in that synthesis step with 4-hydroxypiperidine. ESI-MS: m/z=371.98 [M+H]⁺.

### Intermediate 45: tert-butyl 1-(5-bromopyridin-2-yl)piperidine-4-carboxylate

Following the method for Intermediate 39, replace the tert-butyl 2-(2-hydroxyethoxy)acetate in the synthesis step with tert-butyl piperidine-4-carboxylate to obtain Intermediate 45. ESI-MS: m/z=341.01 [M+H]⁺.

### Intermediate 46: tert-butyl 1-(5-bromopyrimidin-2-yl)piperidine-4-carboxylate

Following the method for Intermediate 40, replace the tert-butyl 2-(2-hydroxyethoxy)acetate in the synthesis step with tert-butyl piperidine-4-carboxylate to obtain Intermediate 46. ESI-MS: m/z=342.02 [M+H]⁺.

### Intermediate 47: tert-butyl 2-(4-(4-bromo-1H-pyrazol-1-yl)piperidin-1-yl)acetate

Following Synthesis Step 1 for Intermediate 41, replace the 4-piperidinemethanol in the synthesis step with 4-(4-bromo-1H-pyrazol-1-yl)piperidine to obtain Intermediate 47.

ESI-MS: m/z=343.97[M+H]⁺.

### Intermediate 48: tert-butyl 2-(3-((4-bromo-1H-pyrazol-1-yl)methyl)azetidin-1-yl)acetate

Following the method for Intermediate 47, replace the 4-(4-bromo-1H-pyrazol-1-yl)piperidine in the synthesis step with 1-(azetidin-3-ylmethyl)-4-bromo-1H-pyrazole to obtain Intermediate 48. ESI-MS: m/z=330.97[M+H]⁺.

### Intermediate 49: tert-butyl 2-(4-(5-bromopyridin-2-yl)piperazin-1-yl)acetate

Following Synthesis Step 1 for Intermediate 41, replace the 4-piperidinemethanol in the synthesis step with 1-(5-bromopyridin-2-yl)piperazine to obtain Intermediate 49. ESI-MS: m/z=355.94[M+H]⁺.

### Intermediate 50: tert-butyl 2-(4-(5-bromopyrimidin-2-yl)piperazin-1-yl)acetate

Following Synthesis Step 1 for Intermediate 41, replace the 4-piperidinemethanol in the synthesis step with 1-(5-bromopyrimidin-2-yl)piperazine to obtain Intermediate 50. ESI-MS: m/z=356.98[M+H]⁺.

### Intermediate 51: tert-butyl 2-((1R,4R)-5-(5-bromopyridin-2-yl)-2,5-diazabicyclo[2.2.1]heptan-2-yl)acetate

### Synthesis Step 1: (1R,4R)-2-(5-bromopyridin-2-yl)-2,5-diazabicyclo[2.2.1]heptane

Following the method for Intermediate 39, replace the tert-butyl 2-(2-hydroxyethoxy)acetate in the synthesis step with tert-butyl (1R,4R)-2,5-diazabicyclo[2.2.1]heptane-2-carboxylate to obtain tert-butyl (1R,4R)-5-(5-bromopyridin-2-yl)-2,5-diazabicyclo[2.2.1]heptane-2-carboxylate. This is then treated with a hydrogen chloride-ethyl acetate solution to remove the Boc protecting group, obtaining (1R,4R)-2-(5-bromopyridin-2-yl)-2,5-diazabicyclo[2.2.1]heptane.

Synthesis Step 2: tert-butyl 2-((1R,4R)-5-(5-bromopyridin-2-yl)-2,5-diazabicyclo[2.2.1]heptan-2-yl)acetate Following the method for Synthesis Step 1 of Intermediate 41, replace the 4-piperidinemethanol in the synthesis step with (1R,4R)-2-(5-bromopyridin-2-yl)-2,5-diazabicyclo[2.2.1]heptane to obtain Intermediate 51. ESI-MS: m/z=368.01[M+H]⁺.

### Intermediate 52: tert-butyl 2-((1R,4R)-5-(5-bromopyrimidin-2-yl)-2,5-diazabicyclo[2.2.1]heptan-2-yl)acetat

### Synthesis Step 1: (1R,4R)-2-(5-bromopyrimidin-2-yl)-2,5-diazabicyclo[2.2.1]heptane

Following the method for Intermediate 40, replace the tert-butyl 2-(2-hydroxyethoxy)acetate in the synthesis step with tert-butyl (1R,4R)-2,5-diazabicyclo[2.2.1]heptane-2-carboxylate to obtain tert-butyl (1R,4R)-5-(5-bromopyrimidin-2-yl)-2,5-diazabicyclo[2.2.1]heptane-2-carboxylate. Then, treat with a hydrogen chloride-ethyl acetate solution to remove the Boc group, obtaining (1R,4R)-2-(5-bromopyrimidin-2-yl)-2,5-diazabicyclo[2.2.1]heptane.

### Synthesis Step 2: tert-butyl 2-((1R,4R)-5-(5-bromopyrimidin-2-yl)-2,5-diazabicyclo[2.2.1]heptan-2-yl)acetate

Following Synthesis Step 1 for Intermediate 41, replace the 4-piperidinemethanol in the synthesis step with (1R,4R)-2-(5-bromopyrimidin-2-yl)-2,5-diazabicyclo[2.2.1]heptane to obtain Intermediate 52. ESI-MS: m/z=369.06[M+H]⁺.

### Intermediate 53: tert-butyl 2-((1S,4S)-5-(5-bromopyridin-2-yl)-2,5-diazabicyclo[2.2.1]heptan-2-yl)acetate

### Synthesis Step 1: (1S,4S)-2-(5-bromopyridin-2-yl)-2,5-diazabicyclo[2.2.1]heptane

Following the method for Intermediate 39, replace the tert-butyl 2-(2-hydroxyethoxy)acetate in the synthesis step with tert-butyl (1S,4S)-2,5-diazabicyclo[2.2.1]heptane-2-carboxylate to obtain tert-butyl (1S,4S)-5-(5-bromopyridin-2-yl)-2,5-diazabicyclo[2.2.1]heptane-2-carboxylate. Then, treat with a hydrogen chloride-ethyl acetate solution to remove the Boc group, obtaining (1S,4S)-2-(5-bromopyridin-2-yl)-2,5-diazabicyclo[2.2.1]heptane.

### Synthesis Step 2: tert-butyl 2-((1S,4S)-5-(5-bromopyridin-2-yl)-2,5-diazabicyclo[2.2.1]heptan-2-yl)acetate

Following Synthesis Step 1 for Intermediate 41, replace the 4-piperidinemethanol in the synthesis step with (1S,4S)-2-(5-bromopyridin-2-yl)-2,5-diazabicyclo[2.2.1]heptane to obtain Intermediate 53. ESI-MS: m/z=368.01[M+H]⁺.

### Intermediate 54: tert-butyl 2-((1S,4S)-5-(5-bromopyrimidin-2-yl)-2,5-diazabicyclo[2.2.1]heptan-2-yl)acetate

### Synthesis Step 1: (1S,4S)-2-(5-bromopyrimidin-2-yl)-2,5-diazabicyclo[2.2.1]heptane

Following the method for Intermediate 40, replace the tert-butyl 2-(2-hydroxyethoxy)acetate in the synthesis step with tert-butyl (1S,4S)-2,5-diazabicyclo[2.2.1]heptane-2-carboxylate to obtain tert-butyl (1S,4S)-5-(5-bromopyrimidin-2-yl)-2,5-diazabicyclo[2.2.1]heptane-2-carboxylate. Then, treat with a hydrogen chloride-ethyl acetate solution to remove the Boc group, obtaining (1S,4S)-2-(5-bromopyrimidin-2-yl)-2,5-diazabicyclo[2.2.1]heptane.

### Synthesis Step 2: tert-butyl 2-((1S,4S)-5-(5-bromopyrimidin-2-yl)-2,5-diazabicyclo[2.2.1]heptan-2-yl)acetate

Following Synthesis Step 1 for Intermediate 41, replace the 4-piperidinemethanol in the synthesis step with (1S,4S)-2-(5-bromopyrimidin-2-yl)-2,5-diazabicyclo[2.2.1]heptane to obtain Intermediate 54. ESI-MS: m/z=368.01[M+H]⁺.

### Intermediate 55: tert-butyl 2-(3-bromo-5,8-dihydro-1,7-naphthyridin-7(6H)-yl)acetate

Following Synthesis Step 1 for Intermediate 41, replace the 4-piperidinemethanol in the synthesis step with 3-bromo-5,6,7,8-tetrahydro-1,7-naphthyridine to obtain Intermediate 55. ESI-MS: m/z=326.94[M+H]⁺.

### Intermediate 56: tert-butyl 2-(6-bromo-3,4-dihydroisoquinolin-2(1H)-yl)acetate

Following Synthesis Step 1 for Intermediate 41, replace the 4-piperidinemethanol in the synthesis step with 6-bromo-1,2,3,4-tetrahydroisoquinoline to obtain Intermediate 56. ESI-MS: m/z=325.98[M+H]⁺.

### Intermediate 57: N-(4-(6-formylpyridin-3-yl)phenyl)-N-((1R,4R)-4-(quinazolin-2-ylamino)cyclohexyl)acetamide

Following Synthesis Step 1 for Intermediate 14, replace the 4-Boc-1-(5-bromo-2-pyridyl)piperazine in the synthesis step with 5-bromopyridine-2-carbaldehyde to obtain Intermediate 57. ESI-MS: m/z=466.35 [M+H]⁺.

### Intermediate 58: 2-(2-((5-(4-(N-((1R,4R)-4-(quinazolin-2-ylamino)cyclohexyl)acetamido)phenyl)pyridin-2-yl)oxy)ethoxy)acetic acid

Following the method for Intermediate 14, replace the 4-Boc-1-(5-bromo-2-pyridyl)piperazine in the synthesis step with Intermediate 39 and replace the 2N hydrogen chloride-ethyl acetate solution with a 50% trifluoroacetic acid solution to obtain Intermediate 58. ESI-MS: m/z=556.17 [M+H]⁺.

### Intermediate 59: 2-(2-((5-(4-(N-((1R,4R)-4-(quinazolin-2-ylamino)cyclohexyl)acetamido)phenyl)pyrimidin-2-yl)oxy)ethoxy)acetic acid

Following the method for Intermediate 14, replace the 4-Boc-1-(5-bromo-2-pyridyl)piperazine in the synthesis step with intermediate 40 and replace the 2N hydrogen chloride-ethyl acetate solution with a 50% trifluoroacetic acid solution to obtain Intermediate 59. ESI-MS: m/z=557.22 [M+H]⁺.

### Intermediate 60: 2-(4-(((5-(4-(N-((1R,4R)-4-(quinazolin-2-ylamino)cyclohexyl)acetamido)phenyl)pyridin-2-yl)oxy)methyl)piperidin-1-yl)acetic acid

Following the method for Intermediate 14, replace the 4-Boc-1-(5-bromo-2-pyridyl)piperazine in the synthesis step with intermediate 41, and replace the 2N hydrogen chloride-ethyl acetate solution with a 50% trifluoroacetic acid solution to obtain Intermediate 60. ESI-MS: m/z=609.14 [M+H]⁺.

### Intermediate 61: 2-(4-(((5-(4-(N-((1R,4R)-4-(quinazolin-2-ylamino)cyclohexyl)acetamido)phenyl)pyrimidin-2-yl)oxy)methyl)piperidin-1-yl)acetic acid

Following the method for Intermediate 14, replace the 4-Boc-1-(5-bromo-2-pyridyl)piperazine in the synthesis step with intermediate 42, and replace the 2N hydrogen chloride-ethyl acetate solution with a 50% trifluoroacetic acid solution to obtain Intermediate 61. ESI-MS: m/z=610.32[M+H]⁺.

### Intermediate 62: 2-(4-((5-(4-(N-((1R,4R)-4-(quinazolin-2-ylamino)cyclohexyl)acetamido)phenyl)pyridin-2-yl)oxy)piperidin-1-yl)acetic acid

Following the method for Intermediate 14, replace the 4-Boc-1-(5-bromo-2-pyridyl)piperazine in the synthesis step with intermediate 43, and replace the 2N hydrogen chloride-ethyl acetate solution with a 50% trifluoroacetic acid solution to obtain Intermediate 62. ESI-MS: m/z=595.87[M+H]⁺.

### Intermediate 63: 2-(4-((5-(4-(N-((1R,4R)-4-(quinazolin-2-ylamino)cyclohexyl)acetamido)phenyl)pyrimidin-2-yl)oxy)piperidin-1-yl)acetic acid

Following the method for Intermediate 14, replace the 4-Boc-1-(5-bromo-2-pyridyl)piperazine in the synthesis step with intermediate 44, and replace the 2N hydrogen chloride-ethyl acetate solution with a 50% trifluoroacetic acid solution to obtain Intermediate 63. ESI-MS: m/z=596.81[M+H]⁺.

### Intermediate 64: 1-(5-(4-(N-((1R,4R)-4-(quinazolin-2-ylamino)cyclohexyl)acetamido)phenyl)pyridin-2-yl)piperidine-4-carboxylic acid

Following the method for Intermediate 14, replace the 4-Boc-1-(5-bromo-2-pyridyl)piperazine in the synthesis step with intermediate 45, and replace the 2N hydrogen chloride-ethyl acetate solution with a 50% trifluoroacetic acid solution to obtain Intermediate 64. ESI-MS: m/z=565.77[M+H]⁺.

### Intermediate 65: 1-(5-(4-(N-((1R,4R)-4-(quinazolin-2-ylamino)cyclohexyl)acetamido)phenyl)pyrimidin-2-yl)piperidine-4-carboxylic acid

Following the method for Intermediate 14, replace the 4-Boc-1-(5-bromo-2-pyridyl)piperazine in the synthesis step with intermediate 46, and replace the 2N hydrogen chloride-ethyl acetate solution with a 50% trifluoroacetic acid solution to obtain Intermediate 65. ESI-MS: m/z=566.74[M+H]⁺.

### Intermediate 66: 2-(4-(4-(4-(N-((1R,4R)-4-(quinazolin-2-ylamino)cyclohexyl)acetamido)phenyl)-1H-pyrazol-1-yl)piperidin-1-yl)acetic acid

Following the method for Intermediate 14, replace the 4-Boc-1-(5-bromo-2-pyridyl)piperazine in the synthesis step with intermediate 47, and replace the 2N hydrogen chloride-ethyl acetate solution with a 50% trifluoroacetic acid solution to obtain Intermediate 66. ESI-MS: m/z=568.42 [M+H]⁺.

### Intermediate 67: 2-(3-((4-(4-(N-((1R,4R)-4-(quinazolin-2-ylamino)cyclohexyl)acetamido)phenyl)-1H-pyrazol-1-yl)methyl)azetidin-1-yl)acetic acid

Following the method for Intermediate 14, replace the 4-Boc-1-(5-bromo-2-pyridyl)piperazine in the synthesis step with intermediate 48, and replace the 2N hydrogen chloride-ethyl acetate solution with a 50% trifluoroacetic acid solution to obtain Intermediate 67. ESI-MS: m/z=554.36 [M+H]⁺.

### Intermediate 68: 2-(4-(5-(4-(N-((1R,4R)-4-(quinazolin-2-ylamino)cyclohexyl)acetamido)phenyl)pyridin-2-yl)piperazin-1-yl)acetic acid

Following the method for Intermediate 14, replace the 4-Boc-1-(5-bromo-2-pyridyl)piperazine in the synthesis step with intermediate 49, and replace the 2N hydrogen chloride-ethyl acetate solution with a 50% trifluoroacetic acid solution to obtain Intermediate 68. ESI-MS: m/z=580.41 [M+H]⁺.

### Intermediate 69: 2-(4-(5-(4-(N-((1R,4R)-4-(quinazolin-2-ylamino)cyclohexyl)acetamido)phenyl)pyrimidin-2-yl)piperazin-1-yl)acetic acid

Following the method for Intermediate 14, replace the 4-Boc-1-(5-bromo-2-pyridyl)piperazine in the synthesis step with intermediate 50, and replace the 2N hydrogen chloride-ethyl acetate solution with a 50% trifluoroacetic acid solution to obtain Intermediate 69. ESI-MS: m/z=581.44 [M+H]⁺.

### Intermediate 70: 2-((1R,4R)-5-(5-(4-(N-((1R,4R)-4-(quinazolin-2-ylamino)cyclohexyl)acetamido)phenyl)pyridin-2-yl)-2,5-diazabicyclo[2.2.1]heptan-2-yl)acetic acid

Following the method for Intermediate 14, replace the 4-Boc-1-(5-bromo-2-pyridyl)piperazine in the synthesis step with intermediate 51, and replace the 2N hydrogen chloride-ethyl acetate solution with a 50% trifluoroacetic acid solution to obtain Intermediate 70. ESI-MS: m/z=592.27 [M+H]⁺.

### Intermediate 71: 2-((1R,4R)-5-(5-(4-(N-((1R,4R)-4-(quinazolin-2-ylamino)cyclohexyl)acetamido)phenyl)pyrimidin-2-yl)-2,5-diazabicyclo[2.2.1]heptan-2-yl)acetic acid

Following the method for Intermediate 14, replace the 4-Boc-1-(5-bromo-2-pyridyl)piperazine in the synthesis step with intermediate 52, and replace the 2N hydrogen chloride-ethyl acetate solution with a 50% trifluoroacetic acid solution to obtain Intermediate 71. ESI-MS: m/z=592.27 [M+H]⁺.

### Intermediate 72: 2-((1S,4S)-5-(5-(4-(N-((1R,4R)-4-(quinazolin-2-ylamino)cyclohexyl)acetamido)phenyl)pyridin-2-yl)-2,5-diazabicyclo[2.2.1]heptan-2-yl)acetic acid

Following the method for Intermediate 14, replace the 4-Boc-1-(5-bromo-2-pyridyl)piperazine in the synthesis step with intermediate 53, and replace the 2N hydrogen chloride-ethyl acetate solution with a 50% trifluoroacetic acid solution to obtain Intermediate 72. ESI-MS: m/z=592.27 [M+H]⁺.

### Intermediate 73: 2-((1S,4S)-5-(5-(4-(N-((1R,4R)-4-(quinazolin-2-ylamino)cyclohexyl)acetamido)phenyl)pyrimidin-2-yl)-2,5-diazabicyclo[2.2.1]heptan-2-yl)acetic acid

Following the method for Intermediate 14, replace the 4-Boc-1-(5-bromo-2-pyridyl)piperazine in the synthesis step with intermediate 54, and replace the 2N hydrogen chloride-ethyl acetate solution with a 50% trifluoroacetic acid solution to obtain Intermediate 73. ESI-MS: m/z=592.27 [M+H]⁺.

### Intermediate 74: 2-(3-(4-(N-((1R,4R)-4-(quinazolin-2-ylamino)cyclohexyl)acetamido)phenyl)-5,8-dihydro-1,7-naphthyridin-7(6H)-yl)acetic acid

Following the method for Intermediate 14, replace the 4-Boc-1-(5-bromo-2-pyridyl)piperazine in the synthesis step with intermediate 55, and replace the 2N hydrogen chloride-ethyl acetate solution with a 50% trifluoroacetic acid solution to obtain Intermediate 74. ESI-MS: m/z=551.46 [M+H]⁺.

### Intermediate 75: 2-(6-(4-(N-((1R,4R)-4-(quinazolin-2-ylamino)cyclohexyl)acetamido)phenyl)-3,4-dihydroisoquinolin-2(1H)-yl)acetic acid

Following the method for Intermediate 14, replace the 4-Boc-1-(5-bromo-2-pyridyl)piperazine in the synthesis step with intermediate 56, and replace the 2N hydrogen chloride-ethyl acetate solution with a 50% trifluoroacetic acid solution to obtain Intermediate 75. ESI-MS: m/z=550.43 [M+H]⁺.

### Example 101: N-(4-(4-((2-(2,6-dioxopiperidin-3-yl)-1,3-dioxoisoindolin-5-yl)glycyl)piperazin-1-yl)phenyl)-N-((1R,4R)-4-(quinazolin-2-ylamino)cyclohexyl)acetamide

(That is, compound 101. In the following examples 102 to 172, the example number corresponds to the compound number.)

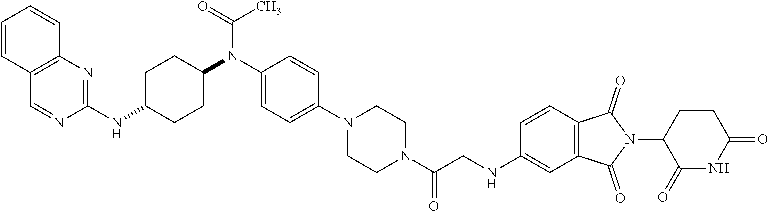

In a 25 mL round-bottom flask, 66.2 mg (0.2 mmol) of intermediate 12 is dissolved in 5 mL of N,N-dimethylformamide. Add 98.8 mg (0.26 mmol) of (7-azabenzotriazol-1-yloxy)-N,N,N′,N′-tetramethyluronium hexafluorophosphate (HATU) and 215.0 μL (1.2 mmol) of N,N-diisopropylethylamine. After stirring at room temperature for ten minutes, add 90.0 mg (0.2 mmol) of intermediate 13 and continue stirring for 1 hour. After TLC shows the complete consumption of the starting material, concentrate the filtrate under reduced pressure and rotary dry. After purification by silica gel column chromatography, 37 mg is obtained, with a yield of 24%.

ESI-MS: m/z=758.41[M+H]⁺; ¹H NMR (400 MHz, Chloroform-d) δ 9.16 (s, 1H), 8.91 (d, J=4.3 Hz, 1H), 7.62 (dt, J=14.9, 6.5 Hz, 3H), 7.51-7.44 (m, 1H), 7.17 (td, J=7.7, 7.2, 2.1 Hz, 1H), 7.05-6.91 (m, 6H), 6.86 (dt, J=8.4, 2.4 Hz, 1H), 5.94 (t, J=4.0 Hz, 1H), 5.52 (d, J=8.1 Hz, 1H), 4.99-4.90 (m, 1H), 4.67 (ddt, J=11.7, 7.5, 3.8 Hz, 1H), 4.14-4.01 (m, 3H), 3.90 (t, J=5.5 Hz, 2H), 3.74-3.64 (m, 3H), 3.30 (dt, J=14.9, 5.5 Hz, 4H), 2.93-2.68 (m, 2H), 2.22-2.10 (m, 2H), 2.04 (s, 2H), 1.88 (d, J=12.0 Hz, 2H), 1.76 (s, 3H), 1.47-1.29 (m, 4H).

### Example 102: N-(4-(6-((4-(2-(2,6-dioxopiperidin-3-yl)-1,3-dioxoisoindolin-5-yl)piperazin-1-yl)methyl)pyridin-3-yl)phenyl)-N-((1R,4R)-4-(quinazolin-2-ylamino)cyclohexyl)acetamide

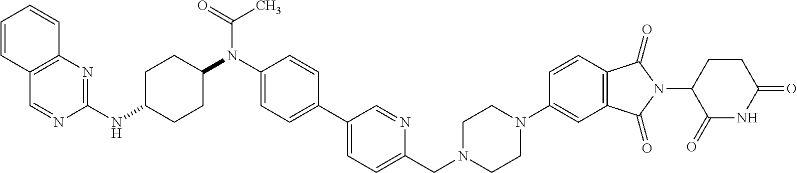

In a 25 mL round-bottom flask, 35.0 mg (0.1 mmol) of intermediate 10 and 47.7 mg (0.1 mmol) of intermediate 57 are added and dissolved in dichloromethane. Add a catalytic amount of acetic acid and stir at room temperature for 1 hour. Then add 43.7 mg (0.21 mmol) of sodium triacetoxyborohydride and stir at room temperature for 6 hours. After TLC shows the complete consumption of the starting material, extract three times with 10 ml of dichloromethane. Concentrate the organic phase under reduced pressure and rotary dry. Purify by silica gel column chromatography to obtain 31 mg, with a yield of 38%.

ESI-MS: m/z=792.57[M+H]; ¹H NMR (400 MHz, Chloroform-d) δ 8.91 (s, 1H), 8.88 (s, 2H), 7.91 (dd, J=8.1, 2.4 Hz, 1H), 7.69 (d, J=8.6 Hz, 1H), 7.66-7.58 (m, 4H), 7.55 (d, J=8.1 Hz, 1H), 7.47 (d, J=8.4 Hz, 1H), 7.29 (d, J=2.3 Hz, 1H), 7.23 (d, J=8.2 Hz, 2H), 7.17 (t, J=7.4 Hz, 1H), 7.06 (dd, J=8.6, 2.3 Hz, 1H), 4.93 (dd, J=12.3, 5.2 Hz, 1H), 4.74 (tt, J=11.6, 3.7 Hz, 1H), 3.80 (s, 2H), 3.77-3.69 (m, 1H), 3.48 (t, J=4.1 Hz, 4H), 2.92-2.77 (m, 3H), 2.75-2.70 (m, 4H), 2.23-2.16 (m, 2H), 2.14-2.09 (m, 2H), 1.98-1.91 (m, 2H), 1.80 (s, 3H), 1.50-1.37 (m, 4H).

### Example 103: N-(4-(6-(4-(2-(2,6-dioxopiperidin-3-yl)-1,3-dioxoisoindolin-5-yl)piperazin-1-yl)pyridin-3-yl)phenyl)-N-((1R,4R)-4-(quinazolin-2-ylamino)cyclohexyl)acetamide

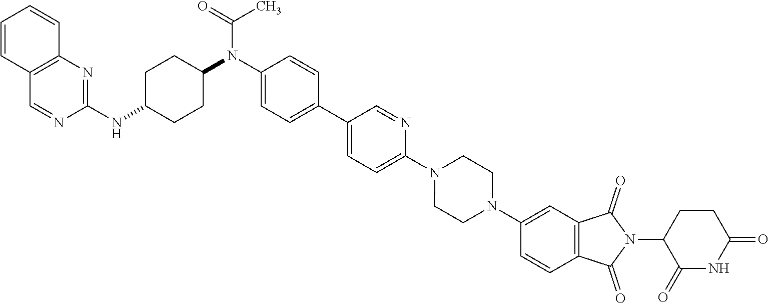

In a 25 mL round-bottom flask, add 156.0 mg (0.3 mmol) of intermediate 14 and 82.8 mg 0.3 mmol) of 2-(2,6-dioxopiperidin-3-yl)-5-fluoroisoindoline-1,3-dione, dissolved in 5 ml of dimethyl sulfoxide solvent. Add 344 μL (1.8 mmol) of N,N-diisopropylethylamine, then heat to 100 °C and stir for 8 hours. After TLC shows the complete consumption of the starting material, concentrate the filtrate under reduced pressure and rotary dry. After purification by silica gel column chromatography, 56 mg is obtained, with a yield of 24%.

ESI-MS: m/z=778.68[M+H]; ¹H NMR (400 MHz, Chloroform-d) δ 8.92 (s, 1H), 8.52 (d, J=2.5 Hz, 1H), 8.50 (s, 1H), 7.81 (dd, J=8.8, 2.6 Hz, 1H), 7.75 (d, J=8.5 Hz, 1H), 7.66-7.55 (m, 4H), 7.49 (d, J=8.6 Hz, 1H), 7.34 (d, J=2.3 Hz, 1H), 7.19 (dd, J=8.4, 6.8 Hz, 3H), 7.11 (dd, J=8.6, 2.3 Hz, 1H), 6.80 (d, J=8.8 Hz, 1H), 5.30 (s, 1H), 4.96 (dd, J=12.2, 5.4 Hz, 1H), 4.79-4.69 (m, 1H), 3.89-3.82 (m, 4H), 3.75 (d, J=7.3 Hz, 1H), 3.67-3.60 (m, 4H), 2.94-2.79 (m, 2H), 2.61 (s, 2H), 2.21 (d, J=11.4 Hz, 2H), 1.99-1.91 (m, 2H), 1.82 (s, 3H), 1.45 (dt, J=18.4, 10.4 Hz, 4H).

### Example 104: N-(4-(4-(2-(2,6-dioxopiperidin-3-yl)-1,3-dioxoisoindolin-5-yl)piperazin-1-yl)phenyl)-N-((1R,4R)-4-(quinazolin-2-ylamino)cyclohexyl)acetamide

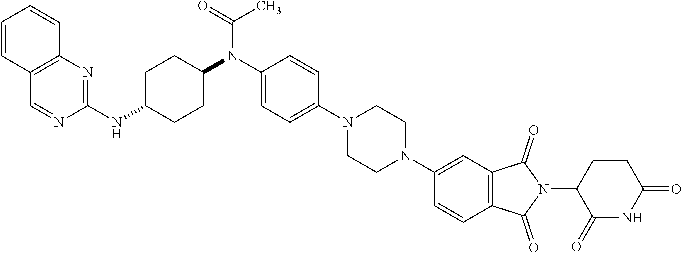

Following the synthesis method of Example 103, replace intermediate 14 with intermediate 13 in the synthesis step. ESI-MS: m/z=701.37 [M+H]; ¹H NMR (400 MHz, Chloroform-d) δ 9.52 (s, 1H), 8.93 (d, J=0.8 Hz, 1H), 7.74 (d, J=8.5 Hz, 1H), 7.65-7.56 (m, 2H), 7.52-7.45 (m, 1H), 7.36 (d, J=2.3 Hz, 1H), 7.19-7.09 (m, 2H), 7.04-6.99 (m, 2H), 6.97-6.91 (m, 2H), 5.66 (d, J=8.1 Hz, 1H), 4.96 (dd, J=12.1, 5.3 Hz, 1H), 4.68 (tt, J=11.7, 3.7 Hz, 1H), 3.72 (dt, J=9.6, 3.8 Hz, 1H), 3.70-3.59 (m, 4H), 3.49-3.40 (m, 4H), 2.90-2.59 (m, 4H), 2.24-2.07 (m, 2H), 1.94-1.84 (m, 2H), 1.76 (s, 3H), 1.49-1.25 (m, 4H).

### Example 105: N-(4-(6-(5-(2-(2,6-dioxopiperidin-3-yl)-1,3-dioxoisoindolin-5-yl)hexahydropyrrolo[3,4-c]pyrrol-2(1H)-yl)pyridin-3-yl)phenyl)-N-((1R,4R)-4-(quinazolin-2-ylamino)cyclohexyl)acetamide

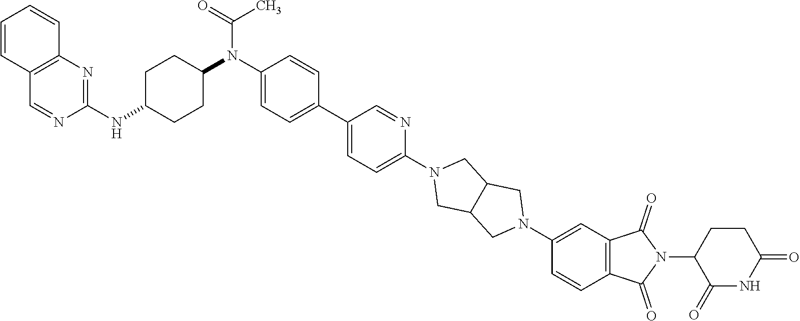

Following the synthesis method of Example 103, replace intermediate 14 with intermediate 15 in the synthesis step.

ESI-MS: m/z=804.44 [M+H]; ¹H NMR (400 MHz, Chloroform-d) δ 9.17 (s, 1H), 8.92 (d, J=0.7 Hz, 1H), 8.49-8.44 (m, 1H), 7.74 (dd, J=8.8, 2.5 Hz, 1H), 7.67 (d, J=8.4 Hz, 1H), 7.64-7.58 (m, 2H), 7.56-7.52 (m, 2H), 7.51-7.44 (m, 1H), 7.20-7.11 (m, 3H), 6.97 (d, J=2.2 Hz, 1H), 6.70 (dd, J=8.5, 2.3 Hz, 1H), 6.49 (d, J=8.8 Hz, 1H), 5.48 (d, J=8.1 Hz, 1H), 4.94 (dd, J=12.1, 5.4 Hz, 1H), 4.72 (ddd, J=11.6, 7.9, 3.5 Hz, 1H), 3.88 (dd, J=10.6, 6.8 Hz, 2H), 3.76 (ddd, J=15.6, 8.9, 5.3 Hz, 3H), 3.56 (dd, J=10.8, 3.6 Hz, 2H), 3.50-3.38 (m, 2H), 3.30-3.25 (m, 2H), 2.93-2.86 (m, 1H), 2.83-2.66 (m, 2H), 2.23-2.15 (m, 2H), 2.14-2.10 (m, 1H), 1.99-1.88 (m, 2H), 1.80 (s, 3H), 1.50-1.35 (m, 4H).

### Example 106: N-(4-(4-((1-(2-(2,6-dioxopiperidin-3-yl)-1,3-dioxoisoindolin-5-yl)piperidin-4-yl)methyl)piperazin-1-yl)phenyl)-N-((1R,4R)-4-(quinazolin-2-ylamino)cyclohexyl)acetamide

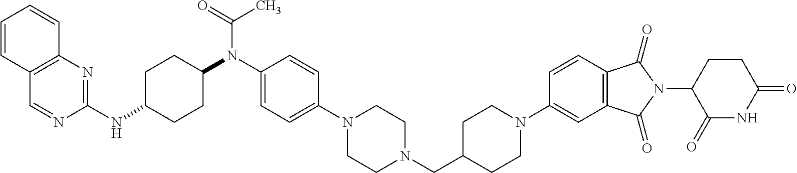

Following the synthesis method of Example 103, replace intermediate 14 with intermediate 16 in the synthesis step.

ESI-MS: m/z=798.62 [M+H]; ¹H NMR (400 MHz, Chloroform-d) δ 8.97 (s, 1H), 8.92 (s, 1H), 7.69-7.58 (m, 3H), 7.48 (d, J=8.5 Hz, 1H), 7.43 (d, J=7.1 Hz, 1H), 7.22 (t, J=6.3 Hz, 2H), 6.85 (d, J=1.9 Hz, 1H), 6.80 (d, J=2.1 Hz, 1H), 6.52 (ddd, J=19.0, 7.7, 2.0 Hz, 2H), 5.43 (s, 1H), 4.93 (dd, J=12.2, 5.4 Hz, 1H), 4.73 (t, J=12.0 Hz, 1H), 4.31 (d, J=7.3 Hz, 2H), 4.16 (t, J=8.1 Hz, 2H), 3.91 (dd, J=8.3, 5.1 Hz, 2H), 3.77-3.70 (m, 1H), 3.47-3.36 (m, 1H), 2.93-2.71 (m, 3H), 2.20 (d, J=11.8 Hz, 2H), 2.15-2.09 (m, 1H), 1.97-1.90 (m, 2H), 1.41 (dq, J=33.2, 12.0 Hz, 4H).

### Example 107: N-(4-(6-(4-(2-(2,6-dioxopiperidin-3-yl)-1,3-dioxoisoindolin-5-yl)piperazin-1-yl)pyrimidin-3-yl)phenyl)-N-((1R,4R)-4-(quinazolin-2-ylamino)cyclohexyl)acetamide

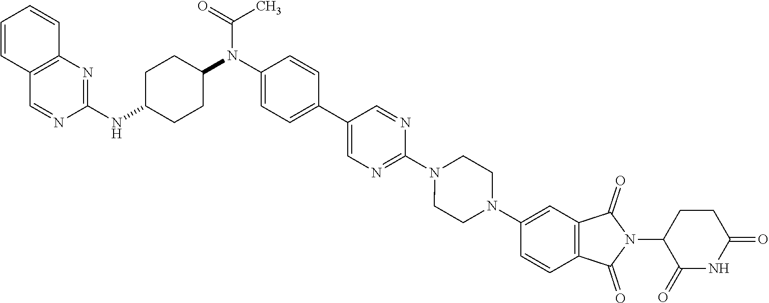

Following the synthesis method of Example 103, replace intermediate 14 with intermediate 17 in the synthesis step.

ESI-MS: m/z=779.70 [M+H]; ¹H NMR (400 MHz, Chloroform-d) δ 9.53 (s, 1H), 8.92 (s, 1H), 8.63 (s, 2H), 7.74 (d, J=8.5 Hz, 1H), 7.61 (dd, J=8.2, 6.5 Hz, 2H), 7.56-7.51 (m, 2H), 7.48 (d, J=8.6 Hz, 1H), 7.35 (d, J=2.3 Hz, 1H), 7.19 (d, J=8.3 Hz, 2H), 7.13 (ddd, J=17.9, 8.2, 1.9 Hz, 2H), 5.65 (d, J=8.1 Hz, 1H), 4.96 (dd, J=12.1, 5.4 Hz, 1H), 4.73 (tt, J=11.7, 3.8 Hz, 1H), 4.11-4.07 (m, 4H), 3.79-3.67 (m, 1H), 3.58 (dd, J=6.5, 4.2 Hz, 4H), 2.95-2.70 (m, 3H), 2.22-2.17 (m, 2H), 2.16 (s, 1H), 1.95-1.90 (m, 2H), 1.80 (s, 3H), 1.48-1.31 (m, 4H).

### Example 108: N-(4-(2-((1-(2-(2,6-dioxopiperidin-3-yl)-1,3-dioxoisoindolin-5-yl)azetidin-3-yl)oxy)pyrimidin-5-yl)phenyl)-N-((1R,4R)-4-(quinazolin-2-ylamino)cyclohexyl)acetamide

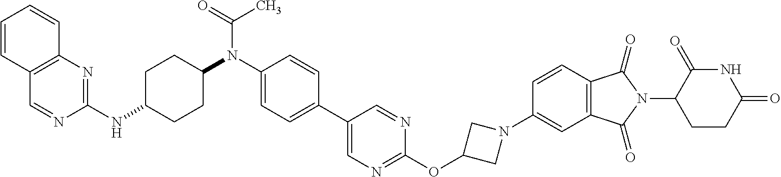

Following the synthesis method of Example 103, replace intermediate 14 with intermediate 18 in the synthesis step.

ESI-MS: m/z=766.37 [M+H]; ¹H NMR (400 MHz, Chloroform-d) δ 9.42 (s, 1H), 8.93 (s, 1H), 8.79 (s, 2H), 7.68 (d, J=8.2 Hz, 1H), 7.63-7.57 (m, 4H), 7.48 (d, J=8.6 Hz, 1H), 7.29-7.23 (m, 2H), 7.21-7.13 (m, 1H), 6.85 (d, J=2.1 Hz, 1H), 6.59 (dd, J=8.3, 2.1 Hz, 1H), 5.64 (tt, J=6.4, 4.2 Hz, 1H), 5.58 (d, J=8.2 Hz, 1H), 4.95 (dd, J=12.1, 5.4 Hz, 1H), 4.75 (tt, J=11.9, 4.0 Hz, 1H), 4.55 (dd, J=9.4, 6.5 Hz, 2H), 4.19 (dd, J=9.6, 4.2 Hz, 2H), 3.80-3.67 (m, 1H), 2.94-2.67 (m, 3H), 2.21 (d, J=6.6 Hz, 2H), 2.15-2.10 (m, 1H), 1.95 (d, J=11.9 Hz, 2H), 1.80 (s, 3H), 1.54-1.33 (m, 4H).

### Example 109: N-(4-(2-(((3R)-1-(2-(2,6-dioxopiperidin-3-yl)-1,3-dioxoisoindolin-5-yl)pyrrolidin-3-yl)oxy)pyrimidin-5-yl)phenyl)-N-((1R,4R)-4-(quinazolin-2-ylamino)cyclohexyl)acetamide

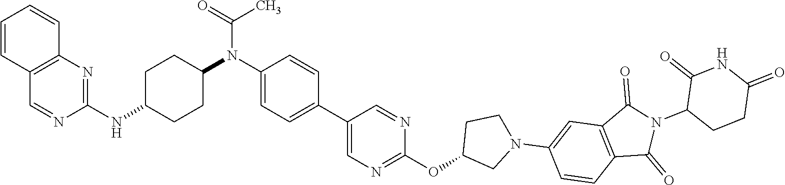

Following the synthesis method of Example 103, replace intermediate 14 with intermediate 19 in the synthesis step.

ESI-MS: m/z=780.45 [M+H]⁺; ¹H NMR (400 MHz, Chloroform-d) δ 8.92 (s, 1H), 8.79 (s, 2H), 8.26 (s, 1H), 7.68 (d, J=8.4 Hz, 1H), 7.64-7.57 (m, 4H), 7.48 (d, J=8.5 Hz, 1H), 7.27 (d, J=1.7 Hz, 1H), 7.26 (s, 1H), 7.23-7.16 (m, 1H), 6.98 (d, J=2.2 Hz, 1H), 6.73 (s, 1H), 5.81 (s, 1H), 5.26 (s, 1H), 4.94 (dd, J=12.2, 5.3 Hz, 1H), 4.81-4.70 (m, 1H), 3.92 (dd, J=11.8, 4.7 Hz, 1H), 3.76 (t, J=10.5 Hz, 3H), 3.65 (t, J=9.1 Hz, 1H), 2.92-2.71 (m, 3H), 2.55 (d, J=7.1 Hz, 1H), 2.47 (ddd, J=12.9, 8.7, 4.2 Hz, 1H), 2.25-2.19 (m, 2H), 2.16-2.10 (m, 1H), 1.97 (s, 2H), 1.81 (s, 3H), 1.51-1.38 (m, 4H).

### Example 110: N-(4-(2-(((3S)-1-(2-(2,6-dioxopiperidin-3-yl)-1,3-dioxoisoindolin-5-yl)pyrrolidin-3-yl)oxy)pyrimidin-5-yl)phenyl)-N-((1R,4R)-4-(quinazolin-2-ylamino)cyclohexyl)acetamide

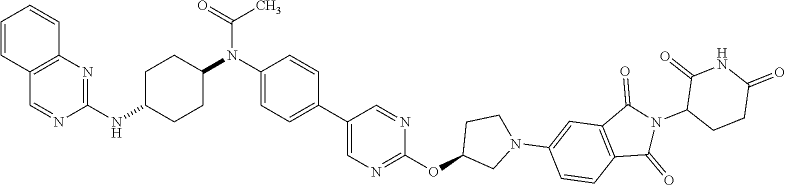

Following the synthesis method of Example 103, replace intermediate 14 with intermediate 20 in the synthesis step.

ESI-MS: m/z=780.45 [M+H]⁺; ¹H NMR (400 MHz, Chloroform-d) δ 9.37 (d, J=5.0 Hz, 1H), 8.93-8.89 (m, 1H), 8.77 (s, 2H), 7.66 (d, J=8.4 Hz, 1H), 7.61-7.56 (m, 4H), 7.50-7.43 (m, 1H), 7.28-7.22 (m, 2H), 7.22-7.12 (m, 1H), 6.97 (d, J=2.2 Hz, 1H), 6.72 (dd, J=8.5, 2.2 Hz, 1H), 5.79 (dt, J=5.1, 3.1 Hz, 1H), 5.52 (d, J=8.2 Hz, 1H), 4.92 (dd, J=12.0, 5.3 Hz, 1H), 4.79-4.68 (m, 1H), 3.90 (dd, J=11.9, 4.7 Hz, 1H), 3.74 (td, J=10.2, 3.0 Hz, 3H), 3.63 (td, J=9.2, 2.5 Hz, 1H), 2.80 (dddd, J=29.6, 20.5, 14.6, 2.8 Hz, 3H), 2.57-2.52 (m, 1H), 2.49-2.39 (m, 1H), 2.20 (d, J=11.9 Hz, 2H), 2.11 (dq, J=10.3, 3.5, 3.1 Hz, 1H), 1.97-1.90 (m, 2H), 1.79 (s, 3H), 1.48-1.32 (m, 4H).

### Example 111: N-(4-(1-((1-(2-(2,6-dioxopiperidin-3-yl)-1,3-dioxoisoindolin-5-yl)piperidin-4-yl)methyl)-2-oxo-1,2-dihydropyridin-4-yl)phenyl)-N-((1R,4R)-4-(quinazolin-2-ylamino)cyclohexyl)acetamide

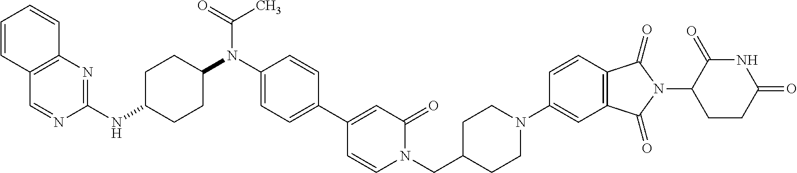

Following the synthesis method of Example 103, replace intermediate 14 with intermediate 21 in the synthesis step.

ESI-MS: m/z=807.67 [M+H]⁺; ¹H NMR (400 MHz, Chloroform-d) δ 9.24 (s, 1H), 8.92 (s, 1H), 7.71-7.57 (m, 5H), 7.48 (dd, J=8.9, 1.4 Hz, 1H), 7.33-7.14 (m, 4H), 7.04 (dd, J=8.7, 2.4 Hz, 1H), 6.85 (d, J=2.0 Hz, 1H), 6.46 (dd, J=7.1, 2.1 Hz, 1H), 5.51 (d, J=8.1 Hz, 1H), 4.94 (dd, J=12.1, 5.4 Hz, 1H), 4.73 (td, J=9.9, 8.2, 5.7 Hz, 1H), 4.03-3.95 (m, 2H), 3.89 (d, J=7.2 Hz, 2H), 3.73 (dtt, J=12.1, 8.5, 4.3 Hz, 1H), 2.98 (td, J=12.8, 2.5 Hz, 2H), 2.93-2.68 (m, 3H), 2.30 (ddt, J=11.5, 7.6, 3.7 Hz, 1H), 2.21 (dd, J=10.0, 5.1 Hz, 2H), 2.12 (ddd, J=10.2, 4.8, 2.7 Hz, 1H), 1.93 (d, J=11.9 Hz, 2H), 1.87-1.82 (m, 2H), 1.79 (s, 3H), 1.74-1.56 (m, 2H), 1.39-1.28 (m, 4H).

### Example 112: N-(4-(1-((1-(2-(2,6-dioxopiperidin-3-yl)-1,3-dioxoisoindolin-5-yl)azetidin-3-yl)methyl)-2-oxo-1,2-dihydropyridin-4-yl)phenyl)-N-((1R,4R)-4-(quinazolin-2-ylamino)cyclohexyl)acetamide

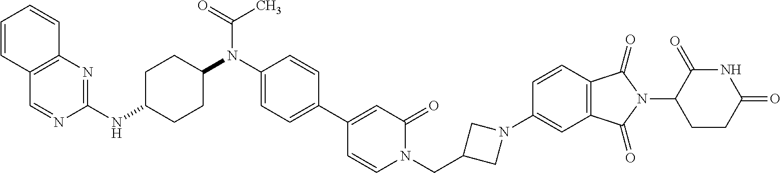

Following the synthesis method of Example 103, replace intermediate 14 with intermediate 22 in the synthesis step.

ESI-MS: m/z=779.52 [M+H]⁺; ¹H NMR (400 MHz, Chloroform-d) δ 8.90 (s, 1H), 8.80 (s, 1H), 7.83 (d, J=0.8 Hz, 1H), 7.72 (d, J=0.8 Hz, 1H), 7.65 (d, J=8.2 Hz, 1H), 7.63-7.59 (m, 2H), 7.52 (d, J=8.2 Hz, 2H), 7.49-7.45 (m, 1H), 7.18 (ddd, J=8.0, 6.9, 1.0 Hz, 1H), 7.11 (d, J=8.4 Hz, 2H), 6.79 (d, J=2.1 Hz, 1H), 6.53 (dd, J=8.3, 2.2 Hz, 1H), 4.92 (dd, J=12.1, 5.4 Hz, 1H), 4.76-4.65 (m, 1H), 4.49 (d, J=7.4 Hz, 2H), 4.17 (t, J=8.1 Hz, 2H), 3.92 (dd, J=8.3, 5.0 Hz, 2H), 3.72 (dt, J=7.2, 3.7 Hz, 1H), 3.40 (ddd, J=12.7, 7.8, 5.0 Hz, 1H), 2.91-2.67 (m, 4H), 2.17 (d, J=11.6 Hz, 2H), 2.12-2.08 (m, 1H), 1.92 (d, J=11.2 Hz, 2H), 1.79 (s, 3H), 1.47 (q, J=12.3 Hz, 4H).

### Example 113: N-(4-(1-(1-(2-(2,6-dioxopiperidin-3-yl)-1,3-dioxoisoindolin-5-yl)azetidin-3-yl)-1H-pyrazol-4-yl)phenyl)-N-((1R,4R)-4-(quinazolin-2-ylamino)cyclohexyl)acetamide

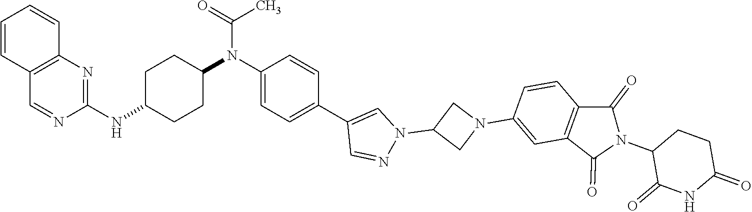

Following the synthesis method of Example 103, replace intermediate 14 with intermediate 23 in the synthesis step.

ESI-MS: m/z=738.28 [M+H]⁺; ¹H NMR (400 MHz, Chloroform-d) δ 9.43 (s, 1H), 8.91 (d, J=0.8 Hz, 1H), 7.89 (dd, J=19.0, 0.8 Hz, 2H), 7.70 (d, J=8.2 Hz, 1H), 7.60 (d, J=7.0 Hz, 2H), 7.53 (d, J=8.4 Hz, 2H), 7.50-7.45 (m, 1H), 7.16 (ddd, J=7.9, 7.0, 1.1 Hz, 1H), 7.11 (d, J=8.4 Hz, 2H), 6.90 (d, J=2.1 Hz, 1H), 6.65 (dd, J=8.3, 2.2 Hz, 1H), 5.36 (ddd, J=7.7, 5.3, 2.4 Hz, 1H), 4.95 (dd, J=12.1, 5.4 Hz, 1H), 4.70 (ddd, J=11.9, 8.2, 3.6 Hz, 1H), 4.61-4.46 (m, 4H), 3.72 (dt, J=7.8, 3.7 Hz, 1H), 2.94-2.80 (m, 2H), 2.78-2.70 (m, 1H), 2.61 (s, 2H), 2.18 (d, J=11.7 Hz, 2H), 1.91 (d, J=11.0 Hz, 2H), 1.78 (s, 3H), 1.51-1.35 (m, 4H).

### Example 114: N-(4-(2-(4-(2-(2,6-dioxopiperidin-3-yl)-6-fluoro-1,3-dioxoisoindolin-5-yl)piperazin-1-yl)pyrimidin-5-yl)phenyl)-N-((1R,4R)-4-(quinazolin-2-ylamino)cyclohexyl)acetamide

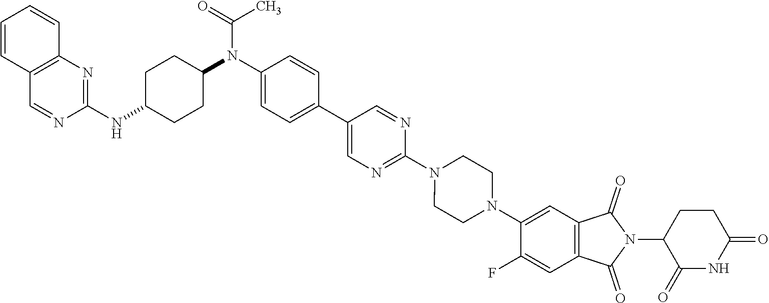

Following the synthesis method of Example 103, in the synthesis step, replace in intermediate 14 with intermediate 17 and replace 2-(2,6-dioxopiperidin-3-yl)-5-fluoroisoindoline-1,3-dione with 2-(2,6-dioxopiperidin-3-yl)-5,6-difluoroisoindoline-1,3-dione.

ESI-MS: m/z=797.65 [M+H]⁺; ¹H NMR (400 MHz, Chloroform-d) δ 9.89 (s, 1H), 8.92 (d, J=0.6 Hz, 1H), 8.62 (s, 2H), 7.63-7.57 (m, 2H), 7.56-7.50 (m, 3H), 7.46 (dd, J=14.0, 7.9 Hz, 2H), 7.19 (d, J=8.2 Hz, 2H), 7.14 (dt, J=7.4, 1.3 Hz, 1H), 5.71 (d, J=11.2 Hz, 1H), 4.99-4.90 (m, 1H), 4.73 (tt, J=11.6, 3.8 Hz, 1H), 4.09 (t, J=5.1 Hz, 4H), 3.77-3.67 (m, 1H), 3.36 (t, J=5.1 Hz, 4H), 2.91-2.71 (m, 3H), 2.23-2.15 (m, 2H), 2.14-2.08 (m, 1H), 1.97-1.88 (m, 2H), 1.81-1.77 (m, 3H), 1.50-1.33 (m, 4H).

### Example 115: N-(4-(1-((1-(2-(2,6-dioxopiperidin-3-yl)-1,3-dioxoisoindolin-5-yl)azetidin-3-yl)methyl)-1H-pyrazol-4-yl)phenyl)-N-((1R,4R)-4-(quinazolin-2-ylamino)cyclohexyl)acetamide

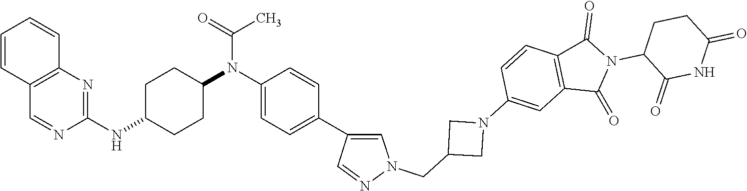

Following the synthesis method of Example 103, replace intermediate 14 with intermediate 24 in the synthesis step.

ESI-MS: m/z=752.21 [M+H]⁺; ¹H NMR (400 MHz, Chloroform-d) δ 8.90 (s, 1H), 8.80 (s, 1H), 7.83 (d, J=0.8 Hz, 1H), 7.72 (d, J=0.8 Hz, 1H), 7.65 (d, J=8.2 Hz, 1H), 7.63-7.59 (m, 2H), 7.52 (d, J=8.2 Hz, 2H), 7.49-7.45 (m, 1H), 7.18 (ddd, J=8.0, 6.9, 1.0 Hz, 1H), 7.11 (d, J=8.4 Hz, 2H), 6.79 (d, J=2.1 Hz, 1H), 6.53 (dd, J=8.3, 2.2 Hz, 1H), 4.92 (dd, J=12.1, 5.4 Hz, 1H), 4.76-4.65 (m, 1H), 4.49 (d, J=7.4 Hz, 2H), 4.17 (t, J=8.1 Hz, 2H), 3.92 (dd, J=8.3, 5.0 Hz, 2H), 3.72 (dt, J=7.2, 3.7 Hz, 1H), 3.40 (ddd, J=12.7, 7.8, 5.0 Hz, 1H), 2.91-2.67 (m, 4H), 2.17 (d, J=11.6 Hz, 2H), 2.12-2.08 (m, 1H), 1.92 (d, J=11.2 Hz, 2H), 1.79 (s, 3H), 1.47 (q, J=12.3 Hz, 4H).

### Example 116: N-(4-(1-(1-(2-(2,6-dioxopiperidin-3-yl)-1,3-dioxoisoindolin-5-yl)pyrrolidin-3-yl)-1H-pyrazol-4-yl)phenyl)-N-((1R,4R)-4-(quinazolin-2-ylamino)cyclohexyl)acetamide

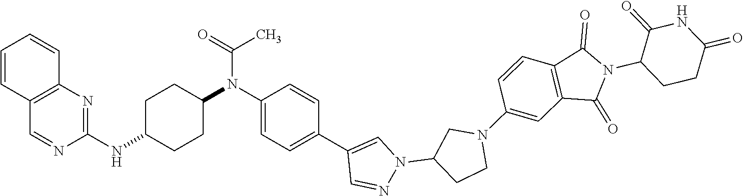

Following the synthesis method of Example 103, replace intermediate 14 with intermediate 25 in the synthesis step.

ESI-MS: m/z=752.23 [M+H]⁺; ¹H NMR (400 MHz, Chloroform-d) δ 8.92 (s, 1H), 8.09 (s, 1H), 7.84 (s, 1H), 7.72 (d, J=8.2 Hz, 2H), 7.62 (d, J=7.9 Hz, 2H), 7.50 (t, J=9.7 Hz, 3H), 7.20 (t, J=7.5 Hz, 1H), 7.11 (d, J=8.1 Hz, 2H), 7.04 (d, J=2.2 Hz, 1H), 6.81-6.76 (m, 1H), 5.32 (d, J=16.9 Hz, 1H), 5.21-5.15 (m, 1H), 4.95 (dd, J=12.2, 5.4 Hz, 1H), 4.71 (t, J=11.8 Hz, 1H), 4.04-3.90 (m, 2H), 3.83-3.60 (m, 3H), 2.94-2.72 (m, 3H), 2.26-2.16 (m, 3H), 2.01 (d, J=6.5 Hz, 1H), 1.93 (d, J=11.6 Hz, 2H), 1.79 (s, 2H), 1.48-1.36 (m, 1H).

### Example 117: N-(4-(1-(1-(2-(2,6-dioxopiperidin-3-yl)-1,3-dioxoisoindolin-5-yl)piperidin-4-yl)-1H-pyrazol-4-yl)phenyl)-N-((1R,4R)-4-(quinazolin-2-ylamino)cyclohexyl)acetamide

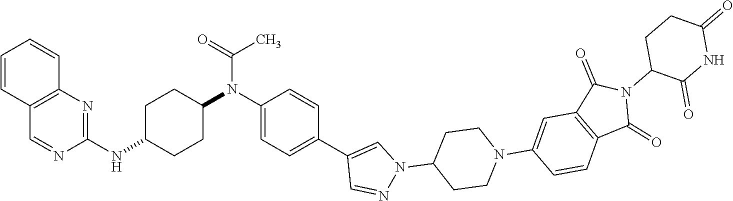

Following the synthesis method of Example 103, replace intermediate 14 with intermediate 26 in the synthesis step.

ESI-MS: m/z=766.47 [M+H]⁺; ¹H NMR (400 MHz, Chloroform-d) δ 9.70 (s, 1H), 8.90 (d, J=0.8 Hz, 1H), 7.82 (d, J=0.8 Hz, 1H), 7.76-7.67 (m, 2H), 7.64-7.55 (m, 2H), 7.54-7.48 (m, 2H), 7.45 (q, J=0.9 Hz, 1H), 7.34 (d, J=2.3 Hz, 1H), 7.18-7.06 (m, 4H), 4.95 (dd, J=12.0, 5.3 Hz, 1H), 4.69 (ddd, J=12.0, 8.4, 3.7 Hz, 1H), 4.46 (ddt, J=11.2, 8.2, 4.1 Hz, 1H), 4.13-4.05 (m, 2H), 3.76-3.64 (m, 1H), 3.19 (t, J=12.5 Hz, 2H), 2.91-2.80 (m, 2H), 2.77-2.69 (m, 1H), 2.32 (d, J=12.6 Hz, 2H), 2.22-2.09 (m, 6H), 1.90 (d, J=11.9 Hz, 2H), 1.76 (s, 3H), 1.51-1.28 (m, 4H).

### Example 118: N-(4-(4-((E)-3-(2-(2,6-dioxopiperidin-3-yl)-1,3-dioxoisoindolin-5-yl)acryloyl)piperazin-1-yl)phenyl)-N-((1R,4R)-4-(quinazolin-2-ylamino)cyclohexyl)acetamide

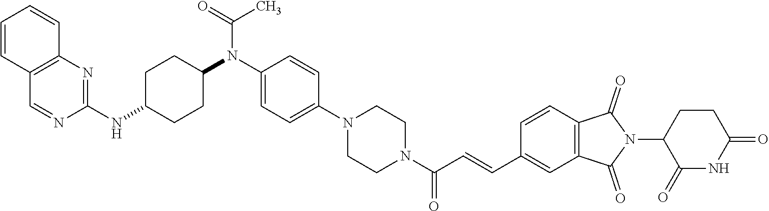

Following the synthesis method of Example 101, replace intermediate 12 with intermediate 11 in the synthesis step.

ESI-MS: m/z=755.22 [M+H]; ¹H NMR (400 MHz, Chloroform-d) δ 9.53 (d, J=4.7 Hz, 1H), 8.92 (d, J=0.8 Hz, 1H), 8.11-8.07 (m, 1H), 7.88 (d, J=11.1 Hz, 1H), 7.79 (d, J=15.4 Hz, 1H), 7.65-7.57 (m, 2H), 7.51-7.45 (m, 1H), 7.17-7.14 (m, 1H), 7.04-6.93 (m, 4H), 5.58 (dd, J=8.8, 3.4 Hz, 1H), 5.06-4.97 (m, 1H), 4.71-4.61 (m, 1H), 4.05 (t, J=6.7 Hz, 2H), 3.91 (d, J=27.4 Hz, 4H), 3.78-3.62 (m, 1H), 3.32 (s, 4H), 2.96-2.71 (m, 3H), 2.20-2.14 (m, 2H), 2.02-1.98 (m, 1H), 1.92-1.81 (m, 2H), 1.75 (s, 3H), 1.59 (tt, J=8.2, 6.5 Hz, 2H), 1.49-1.41 (m, 2H).

### Example 119: N-(4-(2-(2-(2,6-dioxopiperidin-3-yl)-1,3-dioxoisoindolin-5-yl)-2,8-diazaspiro[4.5]decan-8-yl)phenyl)-N-((1R,4R)-4-(quinazolin-2-ylamino)cyclohexyl)acetamide

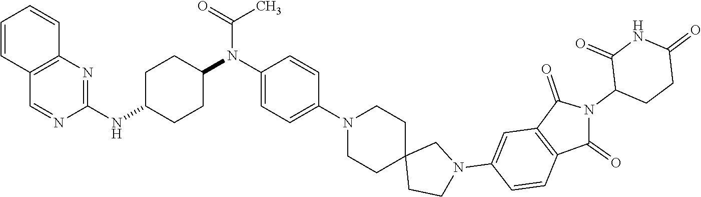

Following the synthesis method of Example 103, replace intermediate 14 with intermediate 27 in the synthesis step.

ESI-MS: m/z=755.16 [M+H]; ¹H NMR (400 MHz, Chloroform-d) δ 8.93 (s, 1H), 8.08 (s, 1H), 7.68 (d, J=8.4 Hz, 1H), 7.63 (d, J=8.5 Hz, 2H), 7.53-7.47 (m, 1H), 7.21 (t, J=7.5 Hz, 1H), 7.00-6.91 (m, 5H), 6.71 (dd, J=8.5, 2.3 Hz, 1H), 5.35 (d, J=6.0 Hz, 1H), 4.94 (dd, J=12.2, 5.4 Hz, 1H), 4.68 (t, J=11.7 Hz, 1H), 3.74 (s, 1H), 3.55 (t, J=7.0 Hz, 2H), 3.41-3.33 (m, 4H), 3.25 (dt, J=12.3, 5.7 Hz, 2H), 2.92-2.80 (m, 2H), 2.24-2.13 (m, 4H), 2.05 (d, J=7.0 Hz, 2H), 1.91 (s, 2H), 1.84 (t, J=5.7 Hz, 4H), 1.77 (s, 3H), 1.49-1.40 (m, 4H).

### Example 120: N-(4-(9-(2-(2,6-dioxopiperidin-3-yl)-1,3-dioxoisoindolin-5-yl)-3,9-diazaspiro[5.5]undecan-3-yl)phenyl)-N-((1R,4R)-4-(quinazolin-2-ylamino)cyclohexyl)acetamide

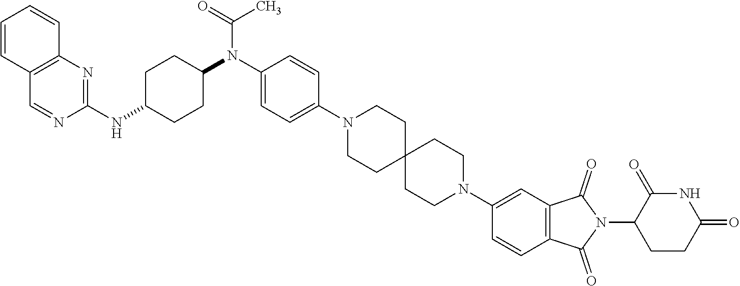

Following the synthesis method of Example 103, replace intermediate 14 with intermediate 28 in the synthesis step.

ESI-MS: m/z=769.46 [M+H]⁺; ¹H NMR (400 MHz, Chloroform-d) δ 9.07 (s, 1H), 8.91 (s, 1H), 7.68 (d, J=8.5 Hz, 1H), 7.61 (ddt, J=8.7, 2.2, 1.4 Hz, 2H), 7.48 (dq, J=8.5, 0.9 Hz, 1H), 7.29 (d, J=2.4 Hz, 1H), 7.17 (ddd, J=8.0, 6.8, 1.1 Hz, 1H), 7.05 (dd, J=8.6, 2.4 Hz, 1H), 6.99-6.90 (m, 4H), 4.94 (dd, J=12.2, 5.4 Hz, 1H), 4.66 (tt, J=11.8, 3.8 Hz, 1H), 3.71 (dt, J=7.5, 3.8 Hz, 1H), 3.47 (dd, J=7.4, 4.4 Hz, 4H), 3.27 (t, J=5.7 Hz, 4H), 2.91-2.69 (m, 3H), 2.20-2.08 (m, 4H), 1.87 (d, J=11.8 Hz, 2H), 1.76 (s, 3H), 1.74-1.69 (m, 8H), 1.50-1.29 (m, 4H).

### Example 121: N-(4-(2-(2-(2,6-dioxopiperidin-3-yl)-1,3-dioxoisoindolin-5-yl)-2,7-diazaspiro[3.5]nonan-7-yl)phenyl)-N-((1R,4R)-4-(quinazolin-2-ylamino)cyclohexyl)acetamide

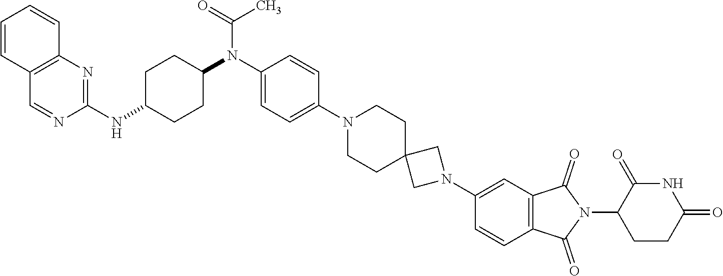

Following the synthesis method of Example 103, replace intermediate 14 with intermediate 29 in the synthesis step.

ESI-MS: m/z=741.31 [M+H]⁺; ¹H NMR (400 MHz, Chloroform-d) δ 8.93 (s, 1H), 8.63 (s, 1H), 7.68-7.61 (m, 3H), 7.50 (dd, J=5.7, 4.2 Hz, 1H), 7.19 (t, J=7.5 Hz, 1H), 7.00-6.90 (m, 4H), 6.81 (d, J=2.1 Hz, 1H), 6.54 (dd, J=8.3, 2.1 Hz, 1H), 4.97-4.89 (m, 1H), 4.67 (tt, J=8.4, 3.9 Hz, 1H), 3.95 (d, J=27.3 Hz, 1H), 3.83 (s, 4H), 3.72 (d, J=8.5 Hz, 1H), 3.26 (dd, J=6.8, 4.2 Hz, 4H), 2.91-2.77 (m, 2H), 2.21-2.09 (m, 4H), 2.02 (t, J=5.6 Hz, 4H), 1.88 (d, J=12.3 Hz, 2H), 1.76 (s, 3H), 1.52-1.40 (m, 4H).

### Example 122: N-(4-(4-(3-(2-(2,6-dioxopiperidin-3-yl)-1,3-dioxoisoindolin-5-yl)prop-2-yn-1-yl)piperazin-1-yl)phenyl)-N-((1R,4R)-4-(quinazolin-2-ylamino)cyclohexyl)acetamide

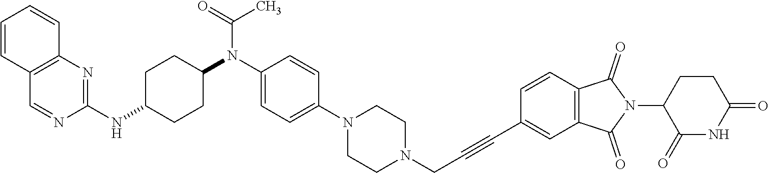

In a 25 mL round-bottom flask, 60.0 mg (0.12 mmol) of intermediate 30, 47.6 mg (0.12 mmol) of 2-(2,6-dioxopiperidin-3-yl)-5-iodoisoindoline-1,3-dione, and 3.0 mg (0.12 mmol) of copper(I) iodide are added and dissolved in anhydrous THF solvent. Add 52.0 μL (0.36 mmol) of triethylamine. After purging with nitrogen three times, add 8.7 mg (0.01 mmol) of Pd(PPh₃)₂Cl₂. Purge with nitrogen three more times and stir at room temperature for 6 hours. After TLC shows the complete consumption of the starting material, extract three times with 20 ml of dichloromethane. Concentrate the organic phase under reduced pressure and rotary dry. Purify by silica gel column chromatography to obtain 34 mg, with a yield of 38%.

ESI-MS: m/z=739.26 [M+H]⁺; ¹H NMR (400 MHz, Chloroform-d) δ 9.96 (d, J=3.5 Hz, 1H), 8.91 (s, 1H), 7.91 (t, J=1.0 Hz, 1H), 7.85-7.74 (m, 2H), 7.59 (ddd, J=10.9, 7.3, 2.0 Hz, 2H), 7.47 (d, J=8.4 Hz, 1H), 7.14 (ddd, J=7.9, 6.8, 1.0 Hz, 1H), 7.00-6.88 (m, 4H), 5.01-4.92 (m, 1H), 4.64 (tt, J=11.8, 3.8 Hz, 1H), 3.70 (qd, J=7.4, 3.6 Hz, 1H), 3.65 (s, 2H), 3.32 (t, J=4.9 Hz, 4H), 2.92-2.63 (m, 7H), 2.17-2.10 (m, 3H), 1.92-1.82 (m, 2H), 1.74 (s, 3H), 1.69-1.59 (m, 1H), 1.46-1.29 (m, 4H).

### Example 123: N-(4-(4-(1-(2-(2,6-dioxopiperidin-3-yl)-1,3-dioxoisoindolin-5-yl)piperidin-4-yl)piperazin-1-yl)phenyl)-N-((1R,4R)-4-(quinazolin-2-ylamino)cyclohexyl)acetamide

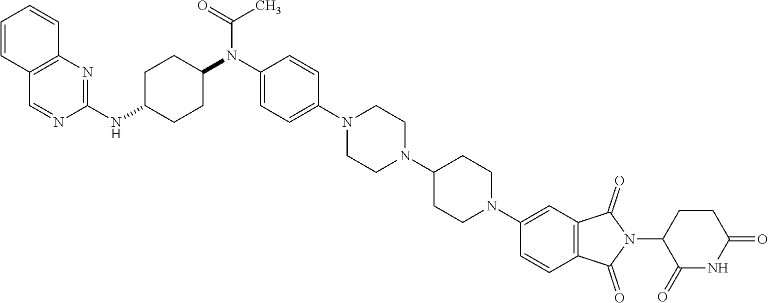

Following the synthesis method of Example 103, replace intermediate 14 with intermediate 31 in the synthesis step.

ESI-MS: m/z=784.39 [M+H]⁺; ¹H NMR (400 MHz, Chloroform-d) δ 8.90 (s, 1H), 8.22 (s, 1H), 7.68 (d, J=8.5 Hz, 1H), 7.61 (dd, J=8.5, 2.9 Hz, 2H), 7.48 (d, J=8.5 Hz, 1H), 7.29 (d, J=2.4 Hz, 1H), 7.18 (t, J=7.4 Hz, 1H), 7.07 (dd, J=8.6, 2.3 Hz, 1H), 6.97 (d, J=8.8 Hz, 2H), 6.90 (d, J=8.9 Hz, 2H), 5.33 (d, J=2.7 Hz, 1H), 4.93 (dd, J=12.3, 5.3 Hz, 1H), 4.71-4.61 (m, 1H), 4.02 (d, J=13.1 Hz, 2H), 3.78-3.67 (m, 1H), 3.32-3.22 (m, 4H), 3.02 (t, J=12.6 Hz, 2H), 2.93-2.83 (m, 2H), 2.82-2.69 (m, 6H), 2.61 (s, 1H), 2.20-2.14 (m, 2H), 2.02 (d, J=7.8 Hz, 2H), 1.86 (s, 2H), 1.75 (s, 3H), 1.72-1.61 (m, 2H), 1.49-1.37 (m, 4H).

### Example 124: N-(4-(4-(1-(2-(2,6-dioxopiperidin-3-yl)-1,3-dioxoisoindolin-5-yl)azetidin-3-yl)piperazin-1-yl)phenyl)-N-((1R,4R)-4-(quinazolin-2-ylamino)cyclohexyl)acetamide

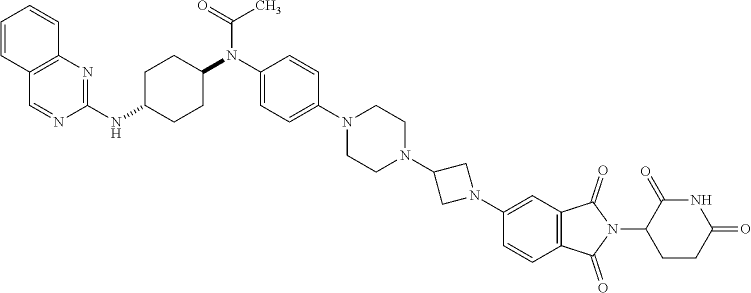

Following the synthesis method of Example 103, replace intermediate 14 with intermediate 32 in the synthesis step. ESI-MS: m/z=756.42 [M+H]⁺.

### Example 125: N-(4-(4-(1-(2-(2,6-dioxopiperidin-3-yl)-1,3-dioxoisoindolin-5-yl)pyrrolidin-3-yl)piperazin-1-yl)phenyl)-N-((1R,4R)-4-(quinazolin-2-ylamino)cyclohexyl)acetamide

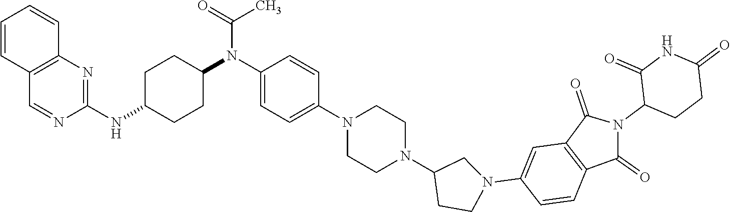

Following the synthesis method of Example 103, replace intermediate 14 with intermediate 33 in the synthesis step. ESI-MS: m/z=770.23 [M+H]; ¹H NMR (400 MHz, Chloroform-d) δ 9.07 (d, J=7.0 Hz, 1H), 8.92 (s, 1H), 7.68 (d, J=8.4 Hz, 1H), 7.62 (td, J=8.4, 1.4 Hz, 2H), 7.49 (dd, J=8.3, 1.2 Hz, 1H), 7.17 (ddd, J=7.9, 6.9, 1.1 Hz, 1H), 7.01-6.97 (m, 3H), 6.93 (d, J=9.1 Hz, 2H), 6.70 (dd, J=8.5, 2.2 Hz, 1H), 5.45 (d, J=8.1 Hz, 1H), 4.94 (dd, J=12.1, 5.3 Hz, 1H), 4.67 (tt, J=11.6, 3.7 Hz, 1H), 3.72 (td, J=9.4, 8.8, 5.7 Hz, 2H), 3.62 (t, J=9.2 Hz, 1H), 3.52-3.42 (m, 1H), 3.41-3.35 (m, 1H), 3.31 (t, J=5.1 Hz, 4H), 3.12 (p, J=7.5 Hz, 1H), 2.92-2.69 (m, 7H), 2.36 (dq, J=15.3, 7.1 Hz, 1H), 2.21-2.15 (m, 2H), 2.13-2.02 (m, 2H), 1.92-1.85 (m, 2H), 1.76 (s, 3H), 1.46-1.35 (m, 4H).

### Example 126: N-(4-(4-((1-(2-(2,6-dioxopiperidin-3-yl)-1,3-dioxoisoindolin-5-yl)pyrrolidin-3-yl)methyl)piperazin-1-yl)phenyl)-N-((1R,4R)-4-(quinazolin-2-ylamino)cyclohexyl)acetamide

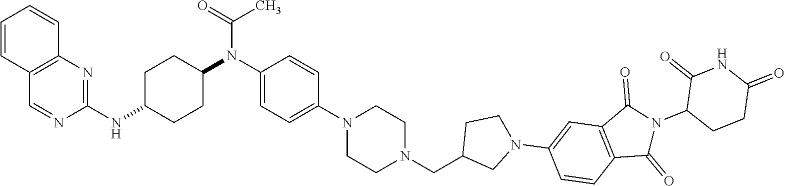

Following the synthesis method of Example 103, replace intermediate 14 with intermediate 34 in the synthesis step.

ESI-MS: m/z=784.30 [M+H]; ¹H NMR (400 MHz, Chloroform-d) δ 9.82 (s, 1H), 8.92 (d, J=0.8 Hz, 1H), 7.65 (d, J=8.4 Hz, 1H), 7.62-7.56 (m, 2H), 7.52-7.45 (m, 1H), 7.15 (ddd, J=8.0, 6.9, 1.1 Hz, 1H), 6.99-6.94 (m, 3H), 6.93-6.87 (m, 2H), 6.68 (dd, J=8.6, 2.2 Hz, 1H), 5.67 (d, J=8.1 Hz, 1H), 4.98-4.89 (m, 1H), 4.66 (tt, J=11.7, 3.8 Hz, 1H), 3.71 (qt, J=7.6, 4.0 Hz, 1H), 3.59 (dd, J=10.2, 7.2 Hz, 1H), 3.54-3.37 (m, 2H), 3.32-3.18 (m, 6H), 2.90-2.60 (m, 8H), 2.50-2.45 (m, 2H), 2.26-2.03 (m, 4H), 1.90-1.83 (m, 2H), 1.75 (s, 3H), 1.47-1.33 (m, 4H).

### Example 127: N-(4-(4-((1-(2-(2,6-dioxopiperidin-3-yl)-1,3-dioxoisoindolin-5-yl)azetidin-3-yl)methyl)piperazin-1-yl)phenyl)-N-((1R,4R)-4-(quinazolin-2-ylamino)cyclohexyl)acetamide

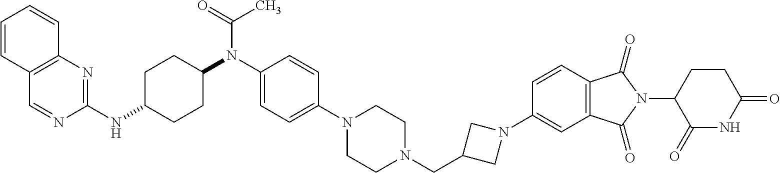

Following the synthesis method of Example 103, replace intermediate 14 with intermediate 35 in the synthesis step.

ESI-MS: m/z=770.28 [M+H]⁺; ¹H NMR (400 MHz, Chloroform-d) δ 9.77 (s, 1H), 8.92 (s, 1H), 7.68-7.58 (m, 3H), 7.49 (dd, J=8.3, 1.0 Hz, 1H), 7.17 (ddd, J=8.0, 6.8, 1.1 Hz, 1H), 7.01-6.94 (m, 2H), 6.91 (dt, J=5.2, 2.9 Hz, 3H), 6.51 (dd, J=8.3, 2.1 Hz, 1H), 5.55 (s, 1H), 4.93 (dd, J=12.0, 5.4 Hz, 1H), 4.67 (tt, J=11.7, 3.7 Hz, 1H), 4.17 (td, J=8.0, 2.2 Hz, 2H), 3.74 (tt, J=8.2, 4.5 Hz, 3H), 3.26 (t, J=5.0 Hz, 4H), 3.09 (tt, J=8.0, 5.7 Hz, 1H), 2.92-2.76 (m, 4H), 2.69 (h, J=5.8 Hz, 4H), 2.23-2.15 (m, 2H), 1.87 (dd, J=10.1, 6.5 Hz, 2H), 1.76 (s, 3H), 1.70-1.52 (m, 2H), 1.38-1.27 (m, 4H).

### Example 128: N—((S)-2,6-dioxopiperidin-3-yl)-5-(4-(N-((1R,4S)-4-(quinazolin-2-ylamino)cyclohexyl)acetamido)phenyl)nicotinamide

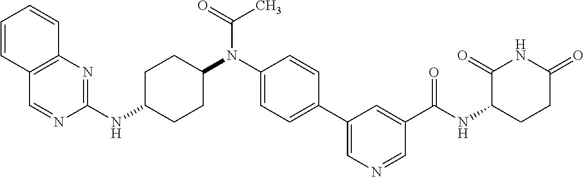

Following the synthesis method of Example 101, replace intermediate 12 with intermediate 36 and intermediate 13 with (S)-3-aminopiperidine-2,6-dione hydrochloride in the synthesis step.

ESI-MS: m/z=592.17 [M+H]⁺; ¹H NMR (400 MHz, DMSO-d₆) δ 8.20 (dq, J=4.8, 2.1 Hz, 1H), 8.14-8.04 (m, 2H), 7.68 (dp, J=4.8, 2.3 Hz, 1H), 6.91 (tt, J=6.8, 3.6 Hz, 2H), 6.84-6.77 (m, 2H), 6.64 (dt, J=8.9, 4.4 Hz, 1H), 6.46 (tt, J=6.4, 3.5 Hz, 2H), 6.38 (td, J=7.4, 6.6, 4.4 Hz, 1H), 4.04 (ddd, J=13.0, 5.8, 3.4 Hz, 1H), 3.83 (p, J=3.8 Hz, 1H), 2.95-2.86 (m, 1H), 2.84-2.75 (m, 1H), 1.97 (dd, J=9.1, 4.6 Hz, 2H), 1.54 (dq, J=9.0, 4.5 Hz, 1H), 1.40-1.28 (m, 2H), 1.17-1.07 (m, 2H), 0.97 (d, J=1.8 Hz, 3H), 0.42-0.08 (m, 4H).

### Example 129: N—((S)-2,6-dioxopiperidin-3-yl)-5-(4-(N-((1R,4S)-4-(quinazolin-2-ylamino)cyclohexyl)acetamido)phenyl)pyridinecarboxamide

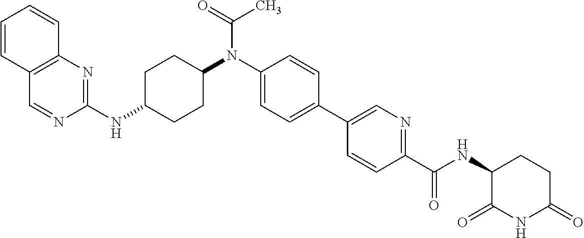

Following the synthesis method of Example 101, in the synthesis step, replace intermediate 12 with intermediate 37 and intermediate 13 with (S)-3-aminopiperidine-2,6-dione hydrochloride.

ESI-MS: m/z=592.17 [M+H]⁺; ¹H NMR (400 MHz, Methanol-d₄) δ 8.94-8.88 (m, 2H), 8.24 (dd, J=8.1, 0.9 Hz, 1H), 8.17 (dd, J=8.1, 2.2 Hz, 1H), 7.80-7.74 (m, 2H), 7.68-7.59 (m, 2H), 7.46 (dd, J=8.3, 1.0 Hz, 1H), 7.36-7.29 (m, 2H), 7.19 (ddd, J=8.1, 7.0, 1.0 Hz, 1H), 4.82 (dd, J=12.8, 5.2 Hz, 1H), 4.71-4.64 (m, 1H), 3.78-3.67 (m, 1H), 3.68-3.61 (m, 1H), 2.85-2.76 (m, 2H), 2.47 (dtd, J=13.0, 4.8, 3.2 Hz, 1H), 2.21-2.16 (m, 2H), 1.98-1.92 (m, 2H), 1.81 (s, 3H), 1.52-1.41 (m, 4H).

### Example 130: N-(4-(2-(4-(5-((2,6-dioxopiperidin-3-yl)amino)-2-fluorophenyl)piperazin-1-yl)pyrimidin-5-yl)phenyl)-N-((1R,4R)-4-(quinazolin-2-ylamino)cyclohexyl)acetamide

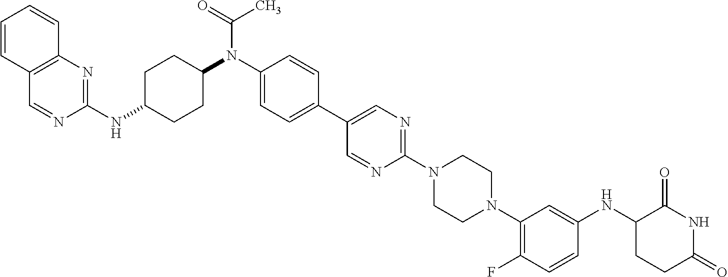

In a 25 mL round-bottom flask, add 83.0 mg (0.13 mmol) of intermediate 38, 75.5 mg (0.39 mmol) of 3-bromopiperidine-2,6-dione, and 65.5 mg (0.79 mmol) of sodium bicarbonate, dissolved in DMF solvent. Heat to 65 °C and stir for 16 hours. After TLC shows the complete consumption of the starting material, extract three times with 20 mL of dichloromethane. Concentrate the organic phase under reduced pressure and rotary dry. Purify by silica gel column chromatography to obtain 33 mg, with a yield of 34%. ESI-MS: m/z=743.57 [M+H]⁺.

### Example 131: (2S,4R)-1-((S)-3,3-dimethyl-2-(2-(2-((5-(4-(N-((1R,4R)-4-(quinazolin-2-ylamino)cyclohexyl)acetamido)phenyl)pyridin-2-yl)oxy)ethoxy)acetamido)butanoyl)-4-hydroxy-N—((S)-1-(4-(4-methylthiazol-5-yl)phenyl)ethyl)pyrrolidine-2-carboxamide

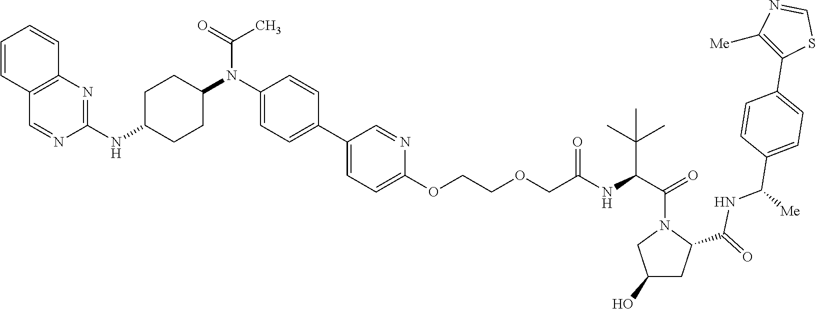

In a 25 mL round-bottom flask, 110.0 mg (0.2 mmol) of intermediate 58 is dissolved in 5 mL of N,N-dimethylformamide. Add 150.6 mg (0.24 mmol) of HATU and 215.0 μL (1.2 mmol) of N,N-diisopropylethylamine. After stirring at room temperature for ten minutes, add 96.2 mg (0.2 mmol) of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (CAS: 1948273-03-7), and continue stirring for 1 hour. After TLC shows the complete consumption of the starting material, concentrate the filtrate under reduced pressure and rotary dry. After purification by silica gel column chromatography, 36 mg is obtained, with a yield of 18%.

ESI-MS: m/z=982.51 [M+H]⁺; ¹H NMR (400 MHz, Chloroform-d) δ 8.92 (s, 1H), 8.52 (d, J=2.5 Hz, 1H), 8.50 (s, 1H), 7.81 (dd, J=8.8, 2.6 Hz, 1H), 7.75 (d, J=8.5 Hz, 1H), 7.66-7.55 (m, 6H), 7.49 (d, J=8.6 Hz, 1H), 7.34 (d, J=2.3 Hz, 1H), 7.19 (dd, J=8.4, 6.8 Hz, 3H), 7.11 (dd, J=8.6, 2.3 Hz, 1H), 6.80 (d, J=8.8 Hz, 1H), 5.16-5.04 (m, 2H), 4.66-4.50 (m, 6H), 4.41 (d, J=5.8 Hz, 2H), 4.10 (d, J=2.5 Hz, 2H), 3.89 (t, J=4.2 Hz, 2H), 2.52 (s, 3H), 2.10 (d, J=13.9 Hz, 2H), 2.01 (d, J=8.4 Hz, 2H), 1.85 (s, 3H), 1.53 (d, J=6.9 Hz, 3H), 1.45-1.41 (m, 2H), 1.34-1.29 (m, 2H), 1.25-1.20 (m, 4H), 1.05 (s, 9H).

### Example 132: (2S,4R)-1-((S)-3,3-dimethyl-2-(2-(2-((5-(4-(N-((1R,4R)-4-(quinazolin-2-ylamino)cyclohexyl)acetamido)phenyl)pyrimidin-2-yl)oxy)ethoxy)acetamido)butanoyl)-4-hydroxy-N—((S)-1-(4-(4-methylthiazol-5-yl)phenyl)ethyl)pyrrolidine-2-carboxamide

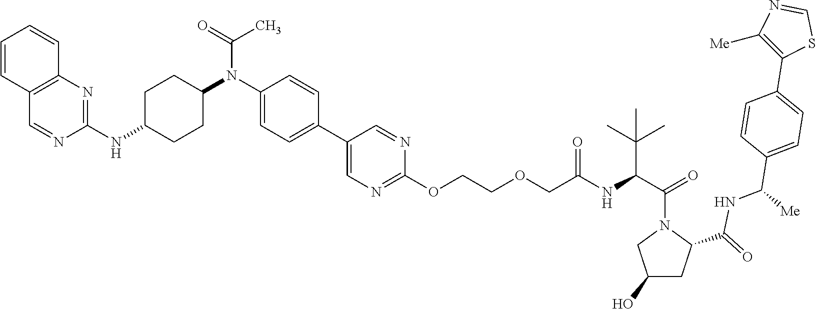

Following the synthesis method of Example 131, replace intermediate 58 with intermediate 59 in the synthesis step.

ESI-MS: m/z=983.49 [M+H]⁺; ¹H NMR (400 MHz, Chloroform-d) δ 8.94-8.90 (m, 1H), 8.70 (s, 1H), 8.65 (s, 2H), 7.66 (d, J=8.4 Hz, 1H), 7.61-7.58 (m, 2H), 7.41-7.38 (m, 1H), 7.30-7.26 (m, 4H), 7.22-7.12 (m, 4H), 6.97 (d, J=2.2 Hz, 1H), 6.72 (dd, J=7.7, 3.6 Hz, 1H), 5.16-5.04 (m, 1H), 4.66-4.52 (m, 6H), 4.36 (d, J=6.3 Hz, 2H), 4.16 (d, J=2.1 Hz, 2H), 3.89 (t, J=3.9 Hz, 2H), 2.51 (s, 3H), 2.07 (d, J=11.0 Hz, 2H), 1.98 (d, J=8.6 Hz, 2H), 1.82 (s, 3H), 1.53 (d, J=6.2 Hz, 3H), 1.45-1.41 (m, 3H), 1.34-1.29 (m, 2H), 1.23-1.17 (m, 4H), 1.03 (s, 9H).

### Example 133: (2S,4R)-1-((S)-3,3-dimethyl-2-(2-(4-(((5-(4-(N-((1R,4R)-4-(quinazolin-2-ylamino)cyclohexyl)acetamido)phenyl)pyridin-2-yl)oxy)methyl)piperidin-1-yl)acetamido)butanoyl)-4-hydroxy-N—((S)-1-(4-(4-methylthiazol-5-yl)phenyl)ethyl)pyrrolidine-2-carboxamide

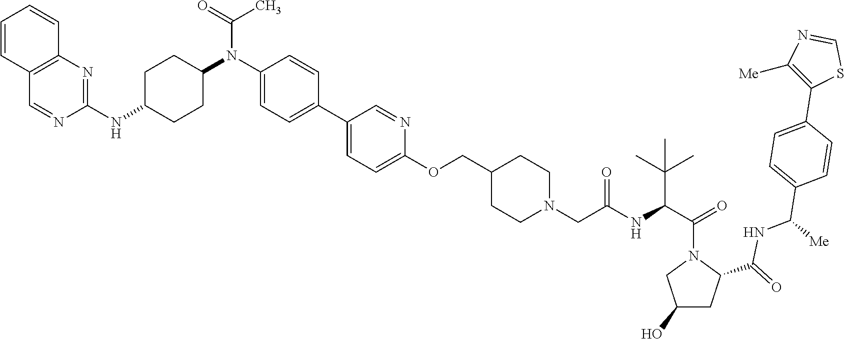

Following the synthesis method of Example 131, replace intermediate 58 with intermediate 60 in the synthesis step.

ESI-MS: m/z=1035.62 [M+H]⁺; ¹H NMR (400 MHz, Chloroform-d) δ 8.81 (d, J=2.2 Hz, 1H), 8.68 (s, 1H), 8.45 (s, 1H), 7.66 (d, J=6.3 Hz, 1H), 7.61 (d, J=8.4 Hz, 2H), 7.42-7.37 (m, 1H), 7.28-7.27 (m, 4H), 7.24-7.17 (m, 5H), 6.97-6.88 (m, J=3.6 Hz, 1H), 6.25 (d, J=8.1 Hz, 1H), 5.07 (p, J=7.0 Hz, 1H), 4.91 (d, J=7.9 Hz, 1H), 4.85 (t, J=8.0 Hz, 1H), 4.70-4.63 (m, 1H), 4.50 (s, 1H), 4.41 (d, J=7.6 Hz, 1H), 4.36-4.28 (m, 4H), 4.24 (d, J=8.3 Hz, 4H), 4.16-4.07 (m, 4H), 3.79 (dd, J=16.2, 3.5 Hz, 1H), 3.09 (d, J=16.2 Hz, 1H), 3.01 (d, J=7.5 Hz, 2H), 2.52 (s, 3H), 2.33 (dd, J=17.3, 7.5 Hz, 2H), 2.12 (d, J=8.6 Hz, 2H), 2.03 (d, J=5.5 Hz, 2H), 1.91 (d, J=8.9 Hz, 2H), 1.78 (s, 3H), 1.47 (d, J=6.9 Hz, 3H), 1.36 (m, J=39.3, 2.9 Hz, 4H), 1.48 (s, 9H).

### Example 134: (2S,4R)-1-((S)-3,3-dimethyl-2-(2-(4-(((5-(4-(N-((1R,4R)-4-(quinazolin-2-ylamino)cyclohexyl)acetamido)phenyl)pyrimidin-2-yl)oxy)methyl)piperidin-1-yl)acetamido)butanoyl)-4-hydroxy-N—((S)-1-(4-(4-methylthiazol-5-yl)phenyl)ethyl)pyrrolidine-2-carboxamide

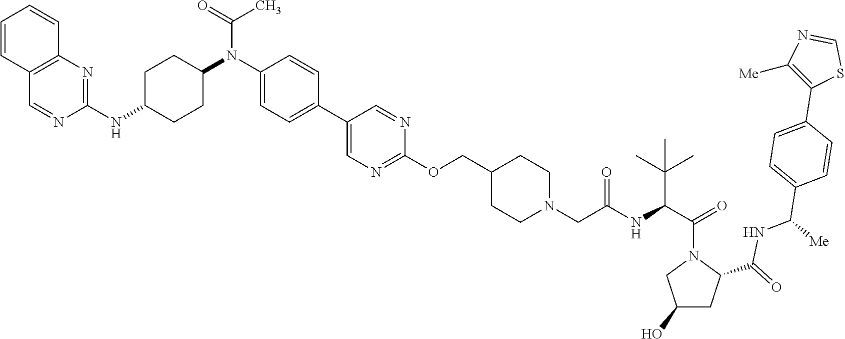

Following the synthesis method of Example 131, replace intermediate 58 with intermediate 61 in the synthesis step.

ESI-MS: m/z=1036.60[M+H]⁺; ¹H NMR (400 MHz, Chloroform-d) δ 8.88-8.74 (m, 1H), 8.68 (s, 1H), 8.45 (s, 2H), 7.66 (d, J=6.3 Hz, 1H), 7.63-7.49 (m, 2H), 7.42-7.37 (m, 1H), 7.28-7.27 (m, 2H), 7.22-7.05 (m, 5H), 6.97-6.88 (m, J=3.6 Hz, 1H), 6.77 (s, 1H), 5.07 (p, J=7.0 Hz, 1H), 4.91 (d, J=7.9 Hz, 1H), 4.85 (t, J=6.0 Hz, 1H), 4.70-4.63 (m, 1H), 4.50 (s, 1H), 4.41 (d, J=7.6 Hz, 1H), 4.38 (s, 3H), 4.24 (d, J=8.3 Hz, 3H), 4.16-4.07 (m, 4H), 3.59 (dd, J=11.4, 3.5 Hz, 1H), 3.20 (dd, J=9.9, 2.0 Hz, 1H), 3.01 (d, J=7.5 Hz, 3H), 2.52 (s, 3H), 2.33 (dd, J=17.3, 7.5 Hz, 2H), 2.54 (s, 3H), 2.05 (d, J=11.0 Hz, 2H), 1.91 (d, J=8.9 Hz, 2H), 1.74 (s, 3H), 1.44 (s, 3H), 1.42-1.28 (m, 4H), 1.06 (s, 9H).

### Example 135: (2S,4R)-1-((S)-3,3-dimethyl-2-(2-(4-((5-(4-(N-((1R,4R)-4-(quinazolin-2-ylamino)cyclohexyl)acetamido)phenyl)pyridin-2-yl)oxy)piperidin-1-yl)acetamido)butanoyl)-4-hydroxy-N—((S)-1-(4-(4-methylthiazol-5-yl)phenyl)ethyl)pyrrolidine-2-carboxamide

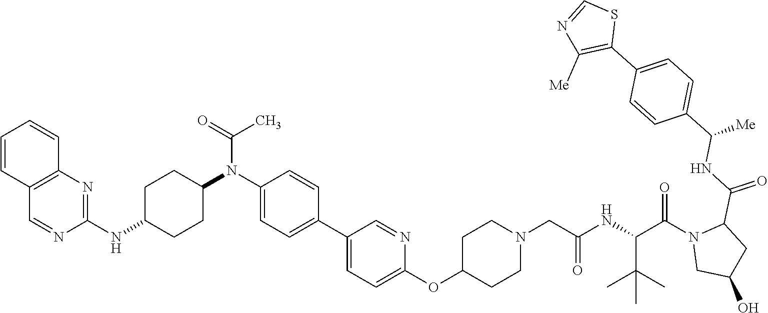

Following the synthesis method of Example 131, replace intermediate 58 with intermediate 62 in the synthesis step.

ESI-MS: m/z=1021.74[M+H]⁺; ¹H NMR (400 MHz, Chloroform-d) δ 8.88 (s, 1H), 8.57 (s, 1H), 8.76 (d, J=2.2 Hz, 1H), 7.97 (d, J=7.3 Hz, 1H), 7.75 (dd, J=12.6, 8.0 Hz, 1H), 7.65-7.59 (m, 2H), 7.57-7.55 (m, 2H), 7.52-7.58 (m, 2H), 7.40-7.33 (m, 4H), 7.24-7.15 (m, 2H), 6.83 (d, J=8.4 Hz, 1H), 5.21 (s, 1H), 5.14 (d, J=8.1 Hz, 1H), 5.06-5.03 (m, 1H), 4.77 (t, J=8.0 Hz, 1H), 4.61-4.55 (m, 1H), 4.48 (d, J=7.7 Hz, 2H), 4.39 (d, J=2.1 Hz, 3H), 4.12 (d, J=11.4 Hz, 1H), 3.63-3.37 (m, 2H), 2.94 (s, 2H), 2.50 (s, 3H), 2.46 (d, J=12.4 Hz, 2H), 2.20 (s, 2H), 2.13-2.03 (m, 4H), 2.50 (s, 3H), 1.79 (s, 3H), 1.50 (d, J=8.5 Hz, 3H), 1.39-1.32 (m, 2H), 1.23-1.18 (m, 4H), 1.06 (s, 9H).

### Example 136: (2S,4R)-1-((S)-3,3-dimethyl-2-(2-(4-((5-(4-(N-((1R,4R)-4-(quinazolin-2-ylamino)cyclohexyl)acetamido)phenyl)pyrimidin-2-yl)oxy)piperidin-1-yl)acetamido)butanoyl)-4-hydroxy-N—((S)-1-(4-(4-methylthiazol-5-yl)phenyl)ethyl)pyrrolidine-2-carboxamide

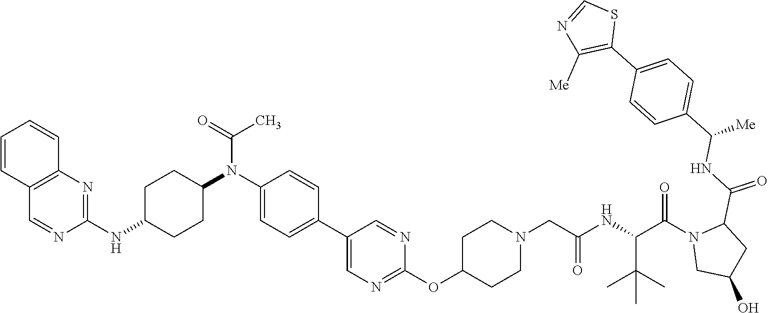

Following the synthesis method of Example 131, replace intermediate 58 with intermediate 63 in the synthesis step.

ESI-MS: m/z=1022.77[M+H]⁺; ¹H NMR (400 MHz, Chloroform-d) δ 8.97 (d, J=6.6 Hz, 1H), 8.86 (s, 1H), 8.77 (s, 2H), 7.65 (d, J=8.4 Hz, 2H), 7.60 (td, J=7.8, 3.5 Hz, 2H), 7.49 (dd, J=8.3, 1.2 Hz, 1H), 7.04-6.97 (m, 5H), 6.93 (d, J=9.1 Hz, 2H), 6.70 (dd, J=8.5, 2.2 Hz, 1H), 5.20 (s, 1H), 5.14 (d, J=6.7 Hz, 1H), 5.06 (p, J=8.6 Hz, 1H), 4.75 (t, J=10.2 Hz, 1H), 4.61 (tt, J=13.5, 5.3 Hz, 1H), 4.48 (d, J=3.3 Hz, 2H), 4.40-4.28 (m, 3H), 4.19 (d, J=11.4 Hz, 1H), 3.56-3.32 (m, 4H), 3.06 (s, 2H), 2.69 (s, 2H), 2.56 (s, 3H), 2.46 (d, J=6.9 Hz, 1H), 2.22-2.16 (m, 2H), 2.13-2.06 (m, 2H), 2.03-1.95 (m, 2H), 1.84 (s, 3H), 1.47 (d, J=6.9 Hz, 3H), 1.33-1.30 (m, 2H), 1.26-1.22 (m, 4H), 1.09 (s, 9H).

### Example 137: N—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-1-(5-(4-(N-((1R,4R)-4-(quinazolin-2-ylamino)cyclohexyl)acetamido)phenyl)pyridin-2-yl)piperidine-4-carboxamide

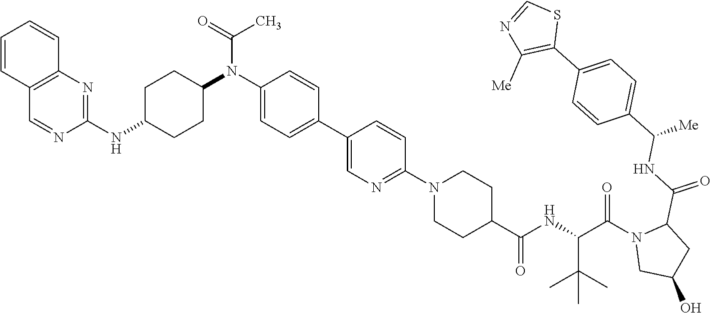

Following the synthesis method of Example 131, replace intermediate 58 with intermediate 64 in the synthesis step.

ESI-MS: m/z=991.67[M+H]⁺; ¹H NMR (400 MHz, Chloroform-d) δ 8.91 (s, 1H), 8.65 (s, 1H), 8.47 (d, J=2.5 Hz, 1H), 7.94 (d, J=8.6 Hz, 1H), 7.76 (dd, J=8.9, 2.6 Hz, 1H), 7.65-7.59 (m, 2H), 7.58-7.54 (m, 2H), 7.51-7.44 (m, 2H), 7.42-7.33 (m, 4H), 7.21-7.13 (m, 2H), 6.77 (d, J=8.8 Hz, 1H), 5.38 (d, J=8.0 Hz, 1H), 5.14-5.02 (m, 1H), 4.71 (t, J=7.8 Hz, 1H), 4.59 (in, J=8.7 Hz, 2H), 4.53-4.49 (m, 1H), 4.46 (t, J=5.9 Hz, 1H), 4.37 (in, J=6.0 Hz, 4H), 4.04 (d, J=11.3 Hz, 1H), 3.66-3.58 (dd, 1H), 3.54 (s, 1H), 2.99-2.86 (in, 2H), 2.51 (s, 3H), 2.49-2.36 (m, 2H), 2.12-2.06 (m, 2H), 2.02-1.87 (m, 4H), 1.84 (s, 3H), 1.49 (d, 3H), 1.41 (in, J=3.1 Hz, 2H), 1.39-1.32 (m, 2H), 1.25 (m, 2H), 1.05 (s, 9H).

### Example 138: N—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-1-(5-(4-(N-((1R,4R)-4-(quinazolin-2-ylamino)cyclohexyl)acetamido)phenyl)pyrimidin-2-yl)piperidine-4-carboxamide

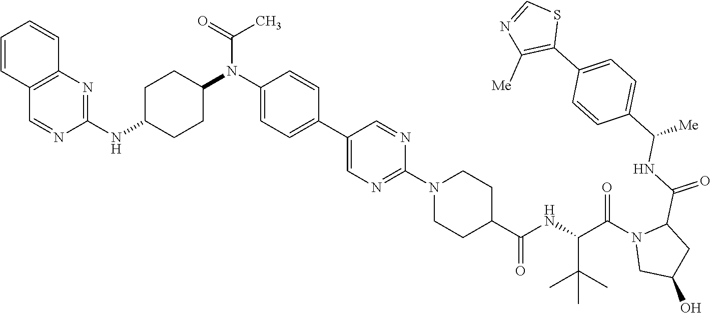

Following the synthesis method of Example 131, replace intermediate 58 with intermediate 65 in the synthesis step.

ESI-MS: m/z=992.65[M+H]; ¹H NMR (400 MHz, Chloroform-d) δ 8.87 (s, 1H), 8.66 (s, 1H), 8.42 (d, J=2.2 Hz, 1H), 7.92 (d, J=8.4 Hz, 1H), 7.76-7.61 (m, 3H), 7.60-7.47 (m, 4H), 7.42-7.33 (m, 4H), 7.19-7.08 (i, 2H), 5.42 (d, J=14.4 Hz, 1H), 5.09-5.00 (m, 1H), 4.76 (t, J 6.9 Hz, 1H), 4.62 (s, 2H), 4.53 (s, 1H), 4.46 (t, J=7.4 Hz, 1H), 4.35 (m, J=8.6 Hz, 4H), 4.17 (d, J=12.5 Hz, 1H), 3.72-3.59 (dd, 1H), 3.61 (s, 1H), 2.91-2.66 (m, 2H), 2.51 (s, 3H), 2.49-2.36 (m, 2H), 2.10 (m, J=15.1, 9.3 Hz, 2H), 2.02-1.87 (m, 4H), 1.84 (s, 3H), 1.49 (d, 3H), 1.45 (m, J=6.9 Hz, 2H), 1.38-1.27 (m, 2H), 1.24 (m, 2H), 1.04 (s, 9H).

### Example 139: (2S,4R)-1-((S)-3,3-dimethyl-2-(2-(44-(4-(N-((1R,4R)-4-(quinazolin-2-ylamino)cyclohexyl)acetamido)phenyl)-1H-pyrazol-1-yl)piperidin-1-yl)acetamido)butanoyl)-4-hydroxy-N—((S)-1-(4-(4-methylthiazol-5-yl)phenyl)ethyl)pyrrolidine-2-carboxamide

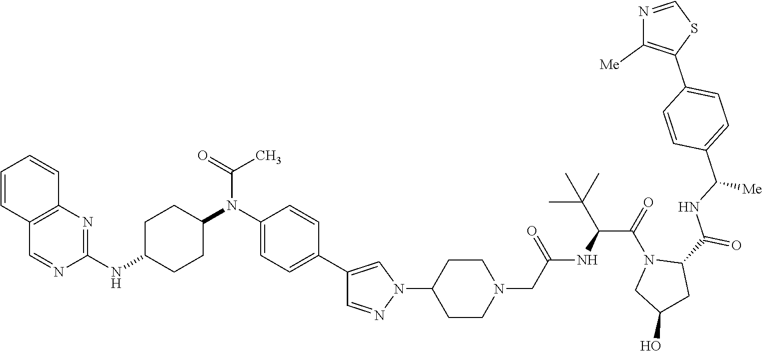

Following the synthesis method of Example 131, replace intermediate 58 with intermediate 66 in the synthesis step.

ESI-MS: m/z=994.47[M+H]; ¹H NMR (400 MHz, Chloroform-d) δ 8.85 (s, 1H), 8.19 (s, 1H), 7.92 (s, 1H), 7.81 (d, J=6.3 Hz, 1H), 7.74 (d, J=12.2 Hz, 1H), 7.71-7.57 (m, 2H), 7.54-7.48 (m, 4H), 7.38-7.31 (m, 4H), 7.28-7.20 (m, 2H), 5.17 (d, J=8.1 Hz, 1H), 5.14-5.02 (m, 1H), 4.75 (t, J=7.8 Hz, 1H), 4.64-4.53 (m, 1H), 4.51 (d, J=8.7 Hz, 2H), 4.41 (dd, J=6.7, 4.8 Hz, 1H), 4.36 (d, J=5.7 Hz, 2H), 4.21-4.10 (m, 2H), 3.61 (dd, J=11.3, 3.8 Hz, 1H), 3.52 (s, 2H), 3.09 (d, J=4.8 Hz, 2H), 3.04 (dt, J=7.5, 3.8 Hz, 4H), 2.59-2.51 (m, 1H), 2.50 (s, 3H), 2.46-2.34 (m, 2H), 2.27-2.16 (m, 4H), 2.13-2.06 (m, 2H), 1.97 (s, 3H), 1.48 (d, J=6.9 Hz, 3H), 1.45-1.31 (m, 4H), 1.08 (s, 9H).

### Example 140: (2S,4R)-1-((R)-3,3-dimethyl-2-(2-(3-((4-(4-(N-((1R,4R)-4-(quinazolin-2-ylamino)cyclohexyl)acetamido)phenyl)-1H-pyrazol-1-yl)methyl)azetidin-1-yl)acetamido)butanoyl)-4-hydroxy-N—((R)-1-(4-(4-methylthiazol-5-yl)phenyl)ethyl)pyrrolidine-2-carboxamide

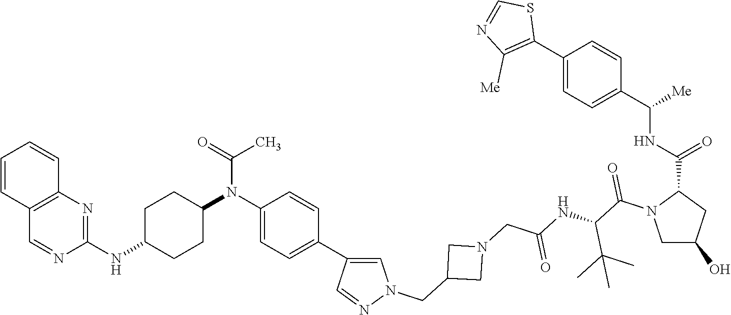

Following the synthesis method of Example 131, replace intermediate 58 with intermediate 67 in the synthesis step.

ESI-MS: m/z=980.51[M+H]; ¹H NMR (400 MHz, Chloroform-d) δ 8.91 (s, 1H), 8.27 (s, 1H), 7.99 (s, 1H), 7.78 (d, J=7.0 Hz, 1H), 7.72 (d, J=8.1 Hz, 1H), 7.68-7.58 (m, 2H), 7.52-7.49 (m, 4H), 7.39-7.30 (m, 4H), 7.25-7.20 (m, 2H), 5.17 (d, J=8.1 Hz, 1H), 4.75 (t, J=6.6 Hz, 1H), 4.60-4.53 (m, 1H), 4.51 (d, J=8.7 Hz, 2H), 4.48 (d, J=6.3 Hz, 2H), 4.41 (d, J=6.8 Hz, 1H), 4.36 (d, J=7.5 Hz, 2H), 4.21-4.08 (m, 2H), 4.02 (d, J=8.0 Hz, 2H), 3.64 (dd, J=11.3, 3.8 Hz, 1H), 3.58 (s, 2H), 3.09 (d, J=5.1 Hz, 2H), 3.04-2.94 (m, 2H), 2.59-2.51 (m, 1H), 2.52 (s, 3H), 2.46-2.31 (m, 2H), 2.00 (s, 2H), 1.78 (s, 3H), 1.48 (d, J=9.4 Hz, 3H), 1.45-1.34 (m, 4H), 1.05 (s, 9H).

### Example 141: (2S,4R)-1-((S)-3,3-dimethyl-2-(2-((1S,4S)-5-(5-(4-(N-((1R,4S)-4-(quinazolin-2-ylamino)cyclohexyl)acetamido)phenyl)pyridin-2-yl)-2,5-diazabicyclo[2.2.1]heptan-2-yl)acetamido)butanoyl)-4-hydroxy-N—((S)-1-(4-(4-methylthiazol-5-yl)phenyl)ethyl)pyrrolidine-2-carboxamide

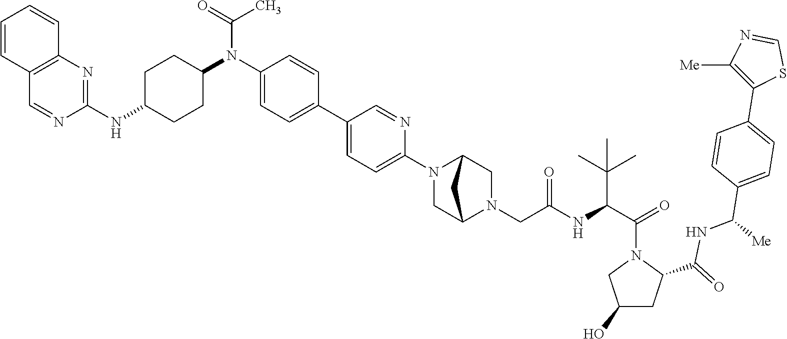

Following the synthesis method of Example 131, replace intermediate 58 with intermediate 72 in the synthesis step.

ESI-MS: m/z=1018.64[M+H]⁺; ¹H NMR (400 MHz, Chloroform-d) δ 8.91 (s, 1H), 8.66 (s, 1H), 8.42 (d, J=2.5 Hz, 1H), 7.91 (d, J=12.3 Hz, 1H), 7.50 (d, J=9.2 Hz, 1H), 7.65-7.61 (m, 2H), 7.60-7.57 (m, 2H), 7.52-7.59 (m, 2H), 7.37-7.24 (m, 4H), 7.21-7.17 (m, 2H), 6.75 (d, J=3.3 Hz, 1H), 5.35-5.28 (m, 1H), 5.07 (p, J=7.1 Hz, 1H), 4.79 (s, 1H), 4.72 (t, J=8.0 Hz, 1H), 4.63-4.52 (m, 2H), 4.48 (dd, J=11.8, 5.7 Hz, 2H), 4.38 (d, J=5.9 Hz, 2H), 4.07 (d, J=11.4 Hz, 1H), 3.81 (s, 1H), 3.71-3.27 (m, 8H), 3.22-2.99 (m, 4H), 2.88 (s, 1H), 2.50 (s, 3H), 2.12-2.06 (m, 2H), 1.99 (d, J=10.7 Hz, 2H), 1.82 (s, 3H), 1.47 (d, J=7.0 Hz, 3H), 1.42-1.35 (m, 4H), 1.05 (s, 9H).

### Example 142: (2S,4R)-1-((S)-3,3-dimethyl-2-(2-((1S,4S)-5-(5-(4-(N-((1R,4S)-4-(quinazolin-2-ylamino)cyclohexyl)acetamido)phenyl)pyrimidin-2-yl)-2,5-diazabicyclo[2.2.1]heptan-2-yl)acetamido)butanoyl)-4-hydroxy-N—((S)-1-(4-(4-methylthiazol-5-yl)phenyl)ethyl)pyrrolidine-2-carboxamide

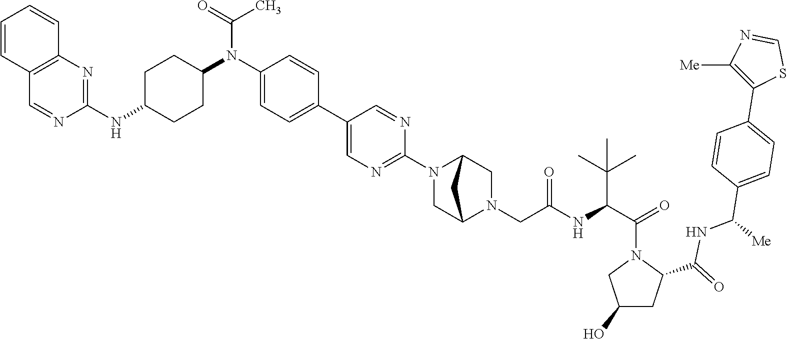

Following the synthesis method of Example 131, replace intermediate 58 with intermediate 73 in the synthesis step. ESI-MS: m/z=1019.64[M+H]⁺.

### Example 143: (2S,4R)-1-((S)-3,3-dimethyl-2-(2-((1R,4R)-5-(5-(4-(N-((1R,4S)-4-(quinazolin-2-ylamino)cyclohexyl)acetamido)phenyl)pyridin-2-yl)-2,5-diazabicyclo[2.2.1]heptan-2-yl)acetamido)butanoyl)-4-hydroxy-N—((S)-1-(4-(4-methylthiazol-5-yl)phenyl)ethyl)pyrrolidine-2-carboxamide

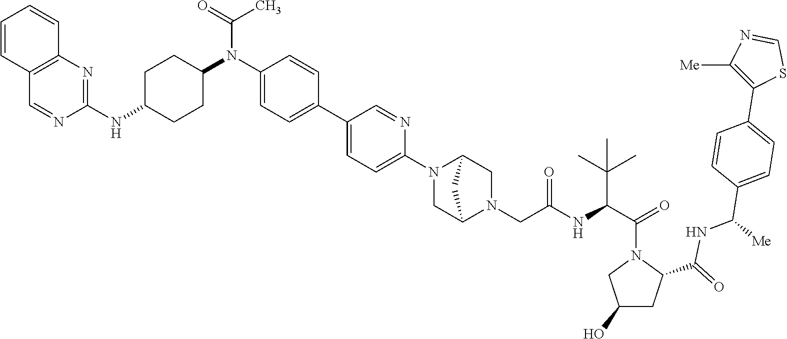

Following the synthesis method of Example 131, replace intermediate 58 with intermediate 70 in the synthesis step.

ESI-MS: m/z=1018.64[M+H]⁺; ¹H NMR (400 MHz, Chloroform-d) δ 8.95 (s, 1H), 8.70 (s, 1H), 8.48 (d, J=4.6 Hz, 1H), 7.85 (d, J=12.5 Hz, 1H), 7.70 (d, J=3.3 Hz, 1H), 7.64-7.60 (m, 2H), 7.55-7.49 (m, 4H), 7.44-7.35 (m, 4H), 7.27-7.14 (m, 2H), 6.87 (d, J=11.3 Hz, 1H), 5.71 (d, J=8.1 Hz, 1H), 5.05 (p, J=7.0 Hz, 1H), 4.76-4.67 (m, 2H), 4.57 (td, J=10.1, 8.3, 5.2 Hz, 1H), 4.52-4.44 (m, 3H), 4.36 (d, J=5.9 Hz, 2H), 4.00 (d, J=11.2 Hz, 1H), 3.64-3.56 (m, 4H), 3.53-3.38 (m, 4H), 3.30-3.13 (m, 2H), 3.06-2.98 (m, 1H), 2.79 (d, J=9.5 Hz, 1H), 2.48 (s, 3H), 2.10-1.92 (m, 4H), 1.88 (s, 3H), 1.45 (d, J=7.0 Hz, 3H), 1.39-1.27 (m, 4H), 1.01 (s, 9H).

### Example 144: (2S,4R)-1-((S)-3,3-dimethyl-2-(2-((1R,4R)-5-(5-(4-(N-((1R,4S)-4-(quinazolin-2-ylamino)cyclohexyl)acetamido)phenyl)pyrimidin-2-yl)-2,5-diazabicyclo[2.2.1]heptan-2-yl)acetamido)butanoyl)-4-hydroxy-N—((S)-1-(4-(4-methylthiazol-5-yl)phenyl)ethyl)pyrrolidine-2-carboxamide

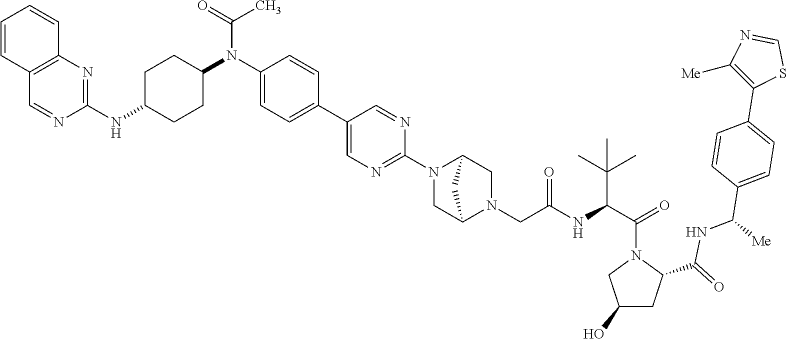

Following the synthesis method of Example 131, replace intermediate with intermediate 71 in the synthesis step. ESI-MS: m/z=1018.64[M+H]⁺.

### Example 145: N—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-4-(5-(4-(N-((1R,4R)-4-(quinazolin-2-ylamino)cyclohexyl)acetamido)phenyl)pyridin-2-yl)piperidine-1-carboxamide

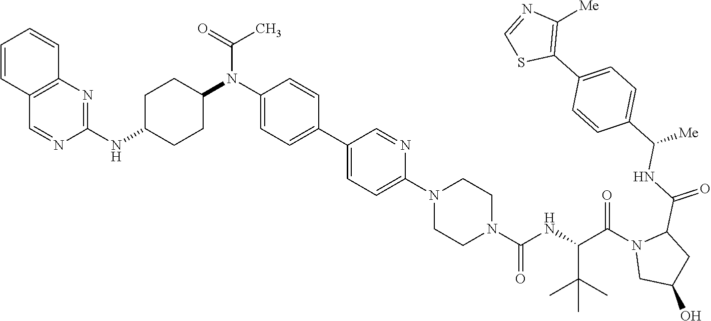

In a 25 mL round-bottom flask, 100 mg (0.19 mmol) of intermediate 14 and 33.9 mg (0.20 mmol) of N,N′-carbonyldiimidazole are added and dissolved in 2 mL of anhydrous dichloromethane. Stir at room temperature for 1 hour. Then, add 91.4 mg (0.19 mmol) of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride, and stir at room temperature for 18 hours. After TLC shows the complete consumption of the starting material, concentrate the filtrate under reduced pressure and rotary dry. After purification by silica gel column chromatography, 36 mg is obtained, with a yield of 19%.

ESI-MS: m/z=992.37[M+H]; ¹H NMR (400 MHz, Chloroform-d) δ 8.87 (s, 1H), 8.63 (s, 1H), 8.49 (d, J=3.3 Hz, 1H), 7.89 (d, J=8.6 Hz, 1H), 7.74 (d, J=8.6 Hz, 1H), 7.65-7.62 (m, 2H), 7.56-7.44 (m, 4H), 7.40-7.31 (m, 4H), 7.27-7.10 (m, 2H), 6.72 (d, J=9.3 Hz, 1H), 5.11-5.06 (m, 2H), 4.78 (t, J=8.1 Hz, 1H), 4.62 (td, J=11.8, 6.0 Hz, 1H), 4.49 (s, 1H), 4.45-4.35 (m, 4H), 4.25 (d, J=11.6 Hz, 1H), 3.71-3.47 (m, 8H), 2.57 (dd, J=8.3, 4.9 Hz, 1H), 2.52 (s, 3H), 2.13-2.07 (m, 2H), 2.00 (d, J=11.0 Hz, 2H), 1.89 (s, 2H), 1.79 (s, 3H), 1.47 (d, J=6.9 Hz, 3H), 1.39-1.28 (m, 4H), 1.08 (s, 9H).

### Example 146: N—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-4-(5-(4-(N-((1R,4R)-4-(quinazolin-2-ylamino)cyclohexyl)acetamido)phenyl)pyrimidin-2-yl)piperazine-1-carboxamide

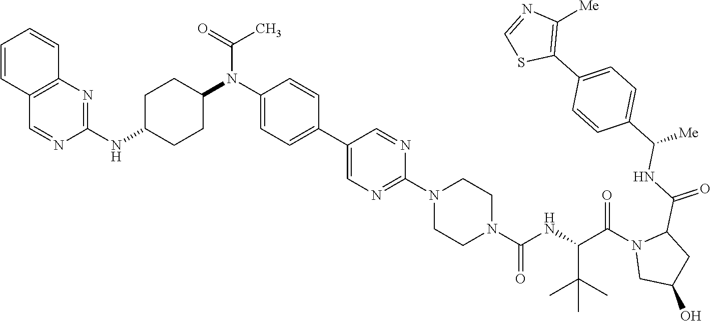

Following the synthesis method of Example 145, replace intermediate 14 with intermediate 17 in the synthesis step. ESI-MS: m/z=993.67[M+H]⁺.

### Example 147: (2S,4R)-1-((S)-3,3-dimethyl-2-(2-(4-(5-(4-(N-((1R,4R)-4-(quinazolin-2-ylamino)cyclohexyl)acetamido)phenyl)pyridin-2-yl)piperazin-1-yl)acetamido)butanoyl)-4-hydroxy-N—((S)-1-(4-(4-methylthiazol-5-yl)phenyl)ethyl)pyrrolidine-2-carboxamide

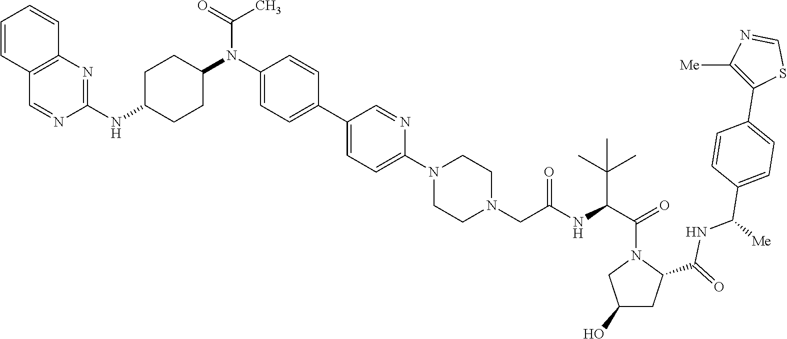

Following the synthesis method of Example 131, replace intermediate 58 with intermediate 68 in the synthesis step.

ESI-MS: m/z=1006.37 [M+H]; ¹H NMR (400 MHz, Chloroform-d) δ 8.91 (s, 1H), 8.65 (s, 1H), 8.47 (d, J=2.5 Hz, 1H), 7.94 (d, J=8.6 Hz, 1H), 7.76 (dd, J=8.9, 2.6 Hz, 1H), 7.65-7.59 (m, 2H), 7.58-7.54 (m, 2H), 7.51-7.44 (m, 2H), 7.42-7.33 (m, 4H), 7.21-7.13 (m, 2H), 6.77 (d, J=8.8 Hz, 1H), 5.29 (s, 1H), 5.08 (p, J=7.0 Hz, 1H), 4.79-4.68 (m, 2H), 4.55-4.48 (m, 2H), 4.20-4.10 (m, 1H), 3.81-3.67 (m, 4H), 3.67-3.56 (m, 2H), 3.19-3.05 (m, 2H), 2.72 (t, J=5.3 Hz, 4H), 2.51 (s, 3H), 2.24-2.17 (m, 4H), 2.13-2.08 (m, 2H), 1.97-1.90 (m, 2H), 1.80 (s, 3H), 1.50 (d, J=7.4 Hz, 3H), 1.41 (s, 4H), 1.08 (s, 9H).

### Example 148: (2S,4R)-1-((S)-3,3-dimethyl-2-(2-(4-(5-(4-(N-((1R,4R)-4-(quinazolin-2-ylamino)cyclohexyl)acetamido)phenyl)pyrimidin-2-yl)piperazin-1-yl)acetamido)butanoyl)-4-hydroxy-N—((S)-1-(4-(4-methylthiazol-5-yl)phenyl)ethyl)pyrrolidine-2-carboxamide

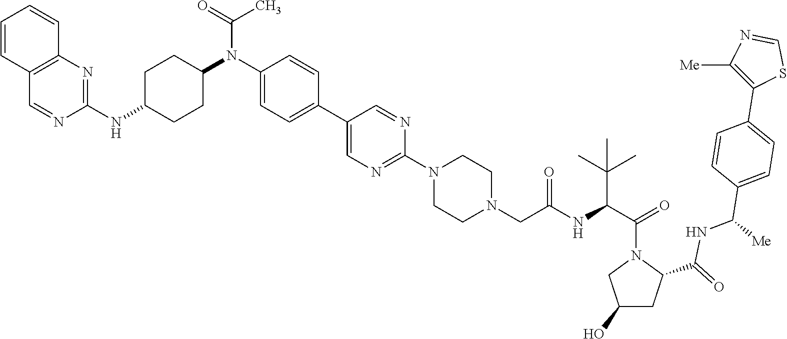

Following the synthesis method of Example 131, replace intermediate 58 with intermediate 69 in the synthesis step.

ESI-MS: m/z=1007.36 [M+H]; ¹H NMR (400 MHz, Chloroform-d) δ 8.87 (s, 1H), 8.44-8.37 (m, 3H), 7.92 (d, J=8.4 Hz, 1H), 7.56-7.40 (m, 2H), 7.46-7.35 (m, 3H), 7.30-7.18 (m, 6H), 6.27 (d, J=8.7 Hz, 1H), 5.31 (s, 1H), 5.08-5.06 (m, 1H), 4.92-4.71 (m, 2H), 4.57-4.52 (m, 2H), 4.28-4.17 (m, 1H), 3.94-3.71 (m, 4H), 3.61 (s, 2H), 3.17-3.12 (m, 2H), 2.72 (t, J=2.7 Hz, 4H), 2.55 (s, 3H), 2.25-2.18 (m, 4H), 2.16-2.06 (m, 2H), 2.00 (d, J=11.7 Hz, 2H), 1.78 (s, 3H), 1.48 (d, J=6.9 Hz, 3H), 1.44-1.36 (m, 4H), 1.05 (s, 9H).

### Example 149: (2S,4R)-1-((S)-3,3-dimethyl-2-(2-(6-(4-(N-((1R,4R)-4-(quinazolin-2-ylamino)cyclohexyl)acetamido)phenyl)-3,4-dihydroisoquinolin-2(1H)-yl)acetamido)butanoyl)-4-hydroxy-N—((S)-1-(4-(4-methylthiazol-5-yl)phenyl)ethyl)pyrrolidine-2-carboxamide

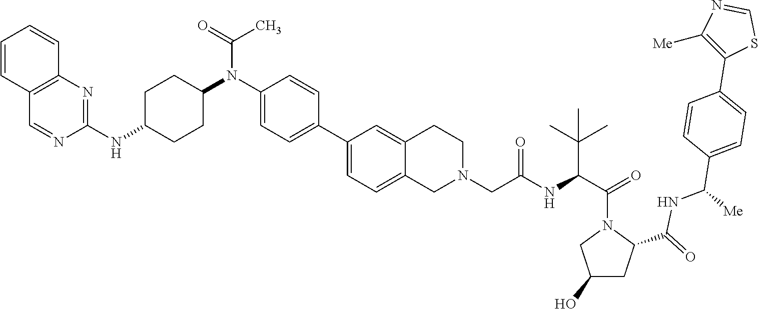

Following the synthesis method of Example 131, replace intermediate 58 with intermediate 75 in the synthesis step. ESI-MS: m/z=976.35[M+H]⁺.

### Example 150: (2S,4R)-1-((S)-3,3-dimethyl-2-(2-(3-(4-(N-((1R,4R)-4-(quinazolin-2-ylamino)cyclohexyl)acetamido)phenyl)-5,8-dihydro-1,7-naphthyridin-7(6H)-yl)acetamido)butanoyl)-4-hydroxy-N—((S)-1-(4-(4-methylthiazol-5-yl)phenyl)ethyl)pyrrolidine-2-carboxamide

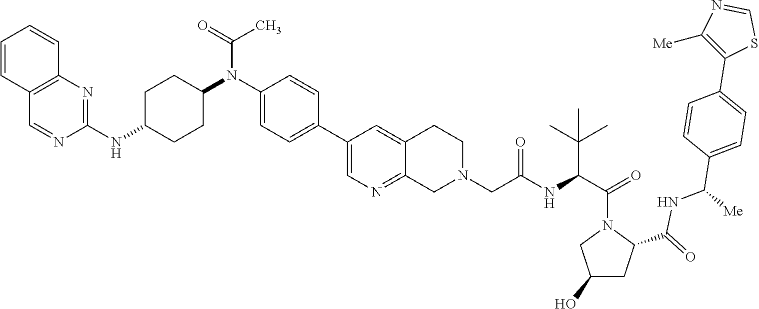

Following the synthesis method of Example 131, replace intermediate 58 with intermediate 74 in the synthesis step. ESI-MS: m/z=977.32[M+H]⁺.

### Example 151: (2S,4R)-4-hydroxy-1-((S)-3-methyl-2-(4-((4-(5-(4-(N-((1R,4R)-4-(quinazolin-2-ylamino)cyclohexyl)acetamido)phenyl)pyridin-2-yl)piperazin-1-yl)methyl)-1H-pyrazol-1-yl)butanoyl)-N—((S)-1-(4-(4-methylthiazol-5-yl)phenyl)ethyl)pyrrolidine-2-carboxamide

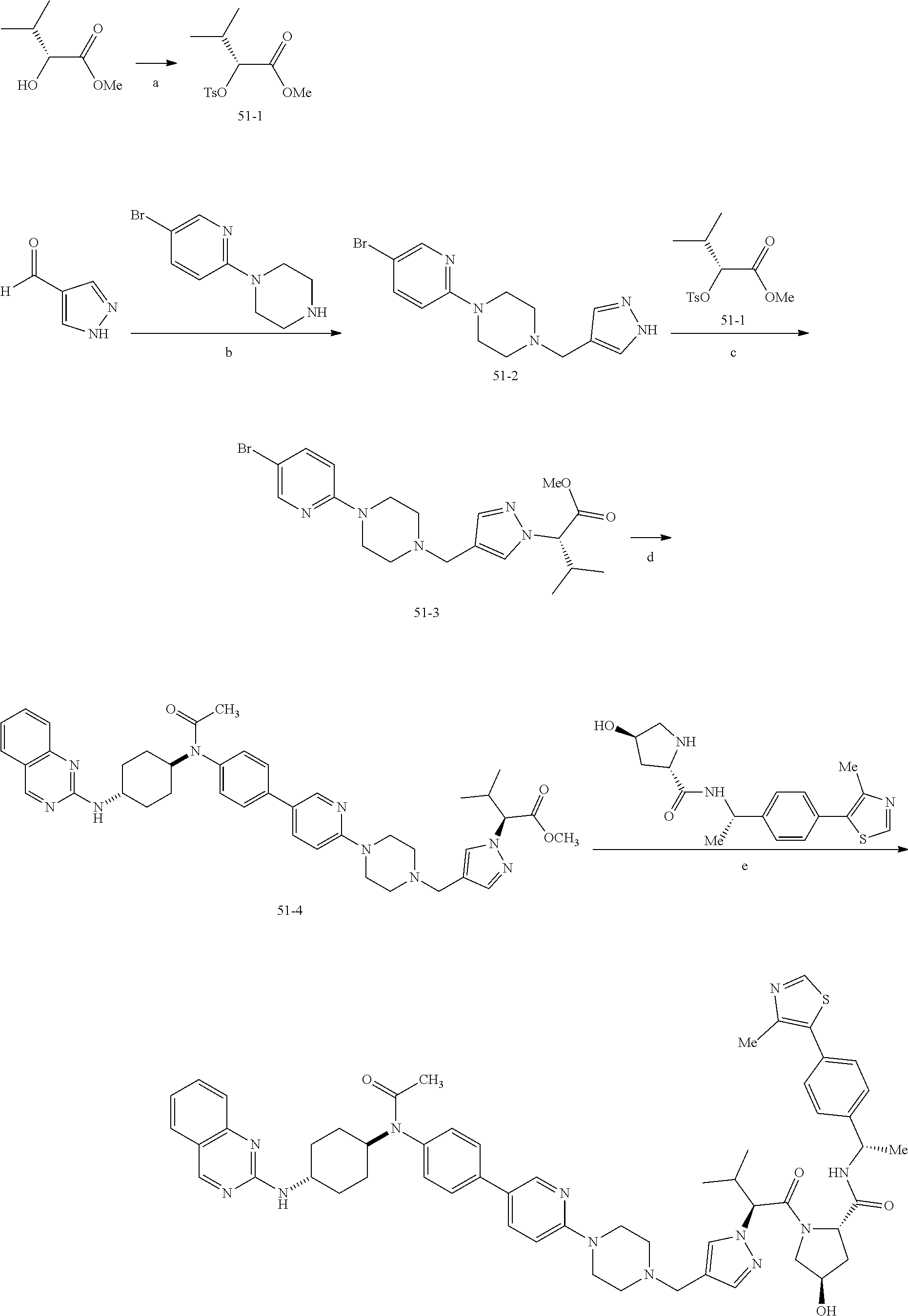

### Synthesis Step a: 51-1

In a 50 mL round-bottom flask, add 1.0 g (5.74 mmol) of methyl (R)-2-hydroxy-3-methylbutanoate, 1.2 g (6.03 mmol) of p-toluenesulfonyl chloride, 2.5 mL (17.2 mmol) of triethylamine, and 6 mg (0.05 mmol) of 4,4-dimethylaminopyridine, dissolved in 20 mL of dichloromethane. After stirring at room temperature for 4 hours, and TLC shows the complete consumption of the starting material, concentrate the filtrate under reduced pressure and rotary dry. Purify by silica gel column chromatography to obtain 864 mg, with a yield of 52%. ESI-MS: m/z=287.07[M+H]⁺.

### Synthesis Step b: 51-2

In a 100 mL round-bottom flask, add 400.0 mg (4.15 mmol) of 1H-pyrazole-4-carbaldehyde and 1.0 g (4.15 mmol) of 1-(5-bromopyridin-2-yl)piperazine, dissolved in 20 mL of dichloromethane. Add a catalytic amount of acetic acid and stir at room temperature for 1 hour. Add 1.8 mg (8.30 mmol) of sodium triacetoxyborohydride and stir at room temperature for 4 hours. After TLC shows the complete consumption of the starting material, extract three times with 30 ml of dichloromethane. Concentrate the organic phase under reduced pressure and rotary dry. Purify by silica gel column chromatography to obtain 1.2 g, with a yield of 92%.

ESI-MS: m/z=321.96[M+H]⁺; ¹H NMR (400 MHz, DMSO-d6) δ 8.14 (d, J=2.6 Hz, 1H), 7.65 (dd, J=9.1, 2.6 Hz, 1H), 7.50 (s, 2H), 6.79 (d, J=9.1 Hz, 1H), 3.46-3.41 (m, 4H), 3.39 (s, 2H), 2.40 (t, J=5.0 Hz, 4H).

### Synthesis Step c: 51-3

In a 50 mL round-bottom flask, add 590.0 mg (1.75 mmol) of intermediate 51-2 and 500.5 mg (1.75 mmol) of intermediate 51-1, dissolved in 20 mL of N,N-dimethylformamide. Add 724.5 mg (5.25 mmol) of potassium carbonate. Heat to 100 °C and stir for 16 hours. After TLC shows the complete consumption of the starting material, extract three times with 30 ml of ethyl acetate. Concentrate the organic phase under reduced pressure and rotary dry. Purify by silica gel column chromatography to obtain 105 mg, with a yield of 14%.

ESI-MS: m/z=436.06[M+H]⁺; ¹H NMR (400 MHz, Chloroform-d) δ 8.16 (dd, J=2.6, 0.7 Hz, 1H), 7.58 (s, 1H), 7.50 (dd, J=9.0, 2.5 Hz, 1H), 7.44 (s, 1H), 6.51 (dt, J=8.9, 1.0 Hz, 1H), 4.61 (d, J=9.7 Hz, 1H), 3.74 (s, 3H), 3.53-3.46 (m, 6H), 2.52 (t, J=5.1 Hz, 4H), 2.50-2.44 (m, 1H), 1.00 (d, J=6.7 Hz, 3H), 0.80 (d, J=6.7 Hz, 3H).

### Synthesis Step d: 51-4

Following Synthesis Step 1 for Intermediate 14, replace the 4-Boc-1-(5-bromo-2-pyridyl)piperazine in the synthesis step with intermediate 51-3 to obtain intermediate 51-4. ESI-MS: m/z=716.34[M+H]⁺.

### Synthesis Step e: Compound 151

In a 50 mL round-bottom flask, add 80.0 mg (0.10 mmol) of intermediate 51-4. After treatment with lithium hydroxide, concentrate under reduced pressure. Add 66.2 mg (0.20 mmol) of (2S,4R)-4-hydroxy-N—((S)-1-(4-(4-methylthiazol-5-yl)phenyl)ethyl)pyrrolidine-2-carboxamide hydrochloride and 49.4 mg (0.1 mmol) of HATU, dissolved in 20 mL of N,N-dimethylformamide. Add 77.4 mg (0.6 mmol) of DIPEA. After stirring at room temperature for 1 hour and TLC shows the complete consumption of the starting material, extract three times with 30 ml of ethyl acetate. Concentrate the organic phase under reduced pressure and rotary dry. Purify by silica gel column chromatography to obtain 20 mg, with a yield of 20%.

ESI-MS: m/z=1015.62[M+H]⁺; ¹H NMR (400 MHz, Chloroform-d) δ 8.94 (s, 1H), 8.64 (s, 1H), 8.48 (t, J=3.3 Hz, 1H), 7.80-7.65 (m, 2H), 7.59 (s, 1H), 7.53 (dd, J=8.4, 2.8 Hz, 2H), 7.43 (s, 1H), 7.40-7.35 (m, 4H), 7.30-7.26 (m, 2H), 7.23-7.16 (m, 4H), 6.70 (t, J=9.2 Hz, 1H), 5.46 (d, J=8.0 Hz, 1H), 5.04 (dt, J=38.0, 7.2 Hz, 1H), 4.81-4.57 (m, 3H), 4.46 (dq, J=10.6, 5.1, 4.3 Hz, 2H), 4.38 (d, J=5.8 Hz, 2H), 3.88-3.70 (m, 1H), 3.61 (s, 4H), 3.51 (d, J=6.8 Hz, 2H), 3.29 (s, 2H), 3.13 (dd, J=10.9, 4.6 Hz, 1H), 2.59 (dt, J=18.9, 4.8 Hz, 4H), 2.50 (d, J=13.0 Hz, 3H), 2.33-2.18 (m, 1H), 2.13-2.06 (m, 2H), 1.99 (d, J=11.4 Hz, 2H), 1.82 (s, 3H), 1.53-1.48 (m, 2H), 1.42 (s, 3H), 1.37-1.27 (m, 4H), 1.07 (dd, J=10.0, 6.6 Hz, 3H), 0.79 (t, J=6.8 Hz, 3H).

### Example 152: (2S,4R)-4-hydroxy-1-((S)-3-methyl-2-(4-(4-(5-(4-(N-((1R,4R)-4-(quinazolin-2-ylamino)cyclohexyl)acetamido)phenyl)pyridin-2-yl)piperazin-1-yl)-1H-pyrazol-1-yl)butanoyl)-N—((S)-1-(4-(4-methylthiazol-5-yl)phenyl)ethyl)pyrrolidine-2-carboxamide

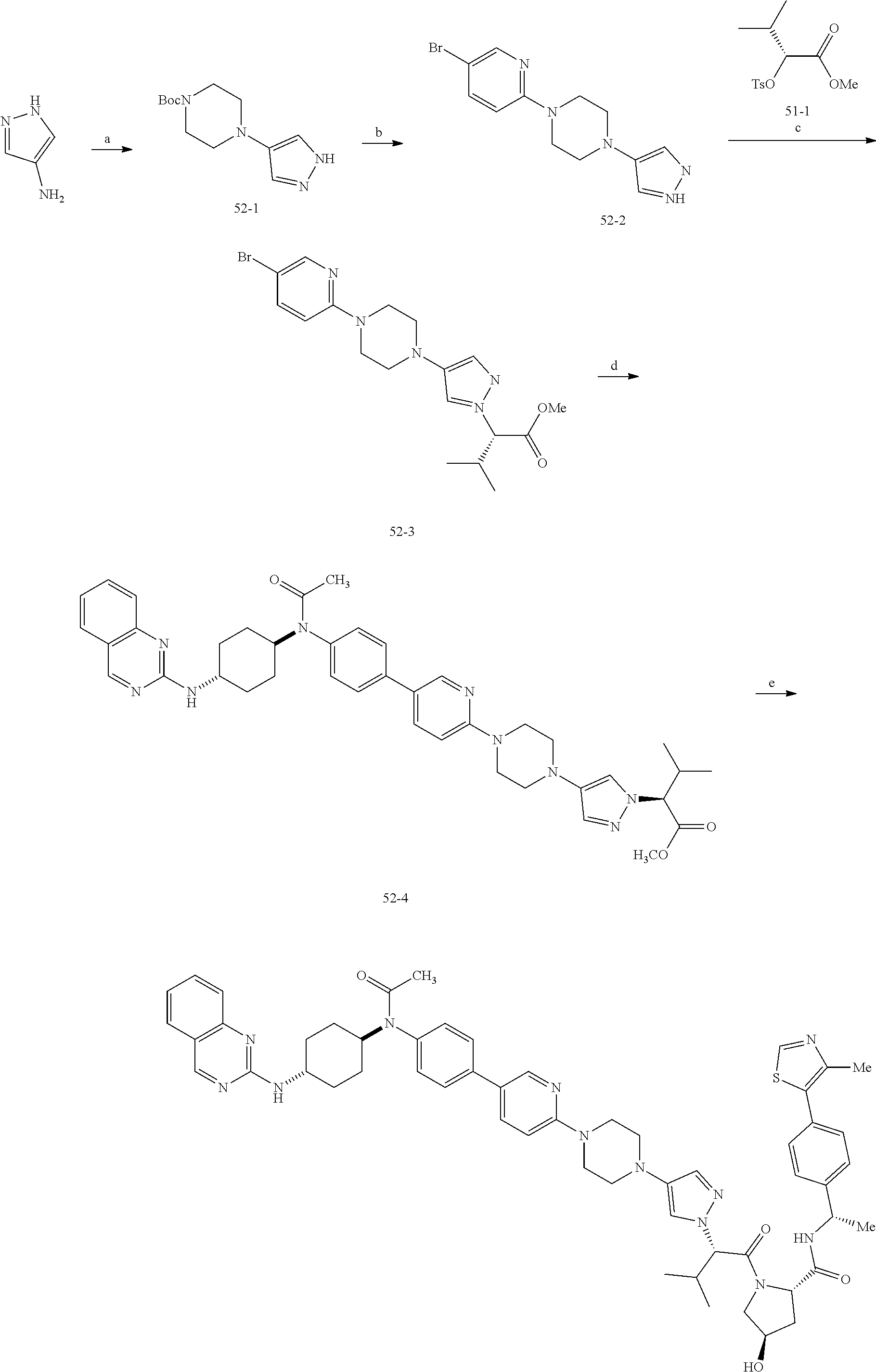

### Synthesis Step a: 52-1

In a 100 mL round-bottom flask, add 1.8 g (22.7 mmol) of 4-aminopyrazole and 11.0 g (45.4 mmol) of tert-butyl N,N-bis(2-chloroethyl)carbamate, dissolved in 30 mL of N,N-dimethylformamide. Add 11.7 g (90.8 mmol) of DIPEA and 3.8 g (22.7 mmol) of potassium iodide. Heat to 50 °C and stir for 18 hours. After TLC shows the complete consumption of the starting material, concentrate the filtrate under reduced pressure and rotary dry. Purify by silica gel column chromatography to obtain 477 mg, with a yield of 8%.

ESI-MS: m/z=253.04[M+H]⁺; ¹H NMR (400 MHz, DMSO-d₆) δ 12.32 (s, 1H), 7.27 (s, 1H), 3.41 (t, J=5.1 Hz, 4H), 2.79 (dd, J=6.0, 4.3 Hz, 4H), 1.40 (s, 9H).

### Synthesis Step b: 52-2

Following the method for Intermediate 39, replace the tert-butyl 2-(2-hydroxyethoxy)acetate in the synthesis step with intermediate 52-1 that has been treated with a hydrochloric acid/ethyl acetate solution. ESI-MS: m/z=307.96[M+H]⁺.

### Synthesis Step c: 52-3

Following Synthesis Step c of Example 151, replace the intermediate 51-2 in the synthesis step with intermediate 52-2.

ESI-MS: m/z=422.25[M+H]⁺; ¹H NMR (400 MHz, Chloroform-d) δ 8.21 (dd, J=2.6, 0.7 Hz, 1H), 7.56 (dd, J=9.0, 2.5 Hz, 1H), 7.27 (d, J=4.0 Hz, 2H), 6.60 (dd, J=9.1, 0.7 Hz, 1H), 4.52 (d, J=9.9 Hz, 1H), 3.74 (s, 3H), 3.66 (t, J=5.2 Hz, 4H), 3.06 (t, J=5.2 Hz, 4H), 2.46 (dp, J=9.9, 6.7 Hz, 1H), 1.00 (d, J=6.7 Hz, 3H), 0.80 (d, J=6.7 Hz, 3H).

### Synthesis Step d: 52-4

Following Synthesis Step 1 for Intermediate 14, replace the 4-Boc-1-(5-bromo-2-pyridyl)piperazine in the synthesis step with intermediate 52-3 to obtain intermediate 52-4. ESI-MS: m/z=702.63[M+H]⁺.

### Synthesis Step e: Compound 152

Following Synthesis Step e of Example 151, replace intermediate 51-4 with intermediate 52-4 in the synthesis step. ESI-MS: m/z=1016.74[M+H]⁺.

### Example 154: (2S,4R)-4-hydroxy-1-((S)-3-methyl-2-(4-(4-(5-(4-(N-((1R,4R)-4-(quinazolin-2-ylamino)cyclohexyl)acetamido)phenyl)pyrimidin-2-yl)piperazin-1-yl)-1H-pyrazol-1-yl)butanoyl)-N—((S)-1-(4-(4-methylthiazol-5-yl)phenyl)ethyl)pyrrolidine-2-carboxamide

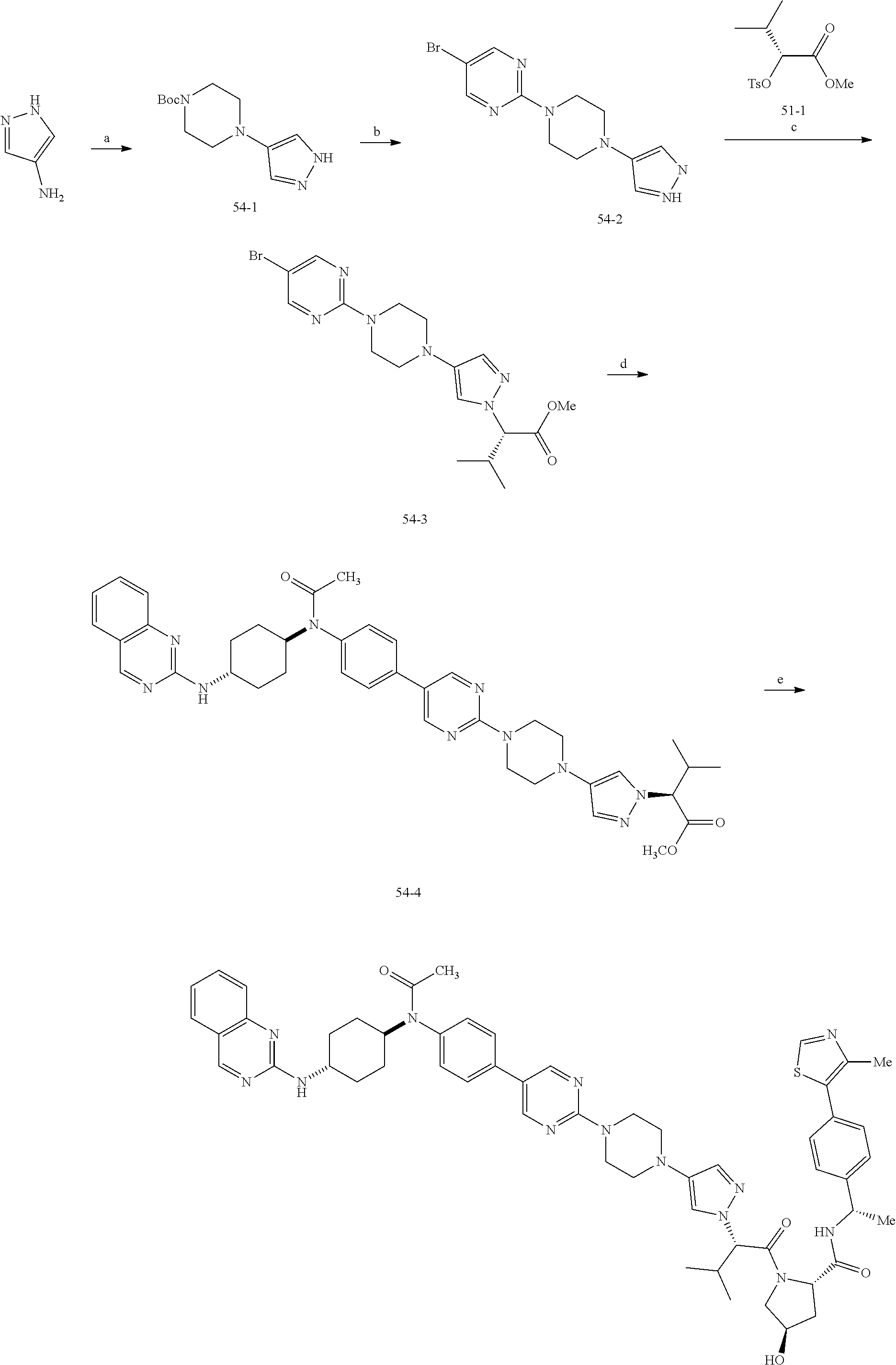

Following the synthesis method of Example 152, wherein Synthesis Step b is replaced by the synthesis method of Intermediate 40, and in the synthesis step, tert-butyl 2-(2-hydroxyethoxy)acetate is replaced with 54-1 that has been treated with a hydrochloric acid/ethyl acetate solution. ESI-MS: m/z=1002.46[M+H]⁺.

### Example 155: (2S,4R)-4-hydroxy-1-((R)-3-methyl-2-(3-(4-(5-(4-(N-((1R,4R)-4-(quinazolin-2-ylamino)cyclohexyl)acetamido)phenyl)pyridin-2-yl)piperazin-1-yl)isoxazol-5-yl)butanoyl)-N—((S)-1-(4-(4-methylthiazol-5-yl)phenyl)ethyl)pyrrolidine-2-carboxamide

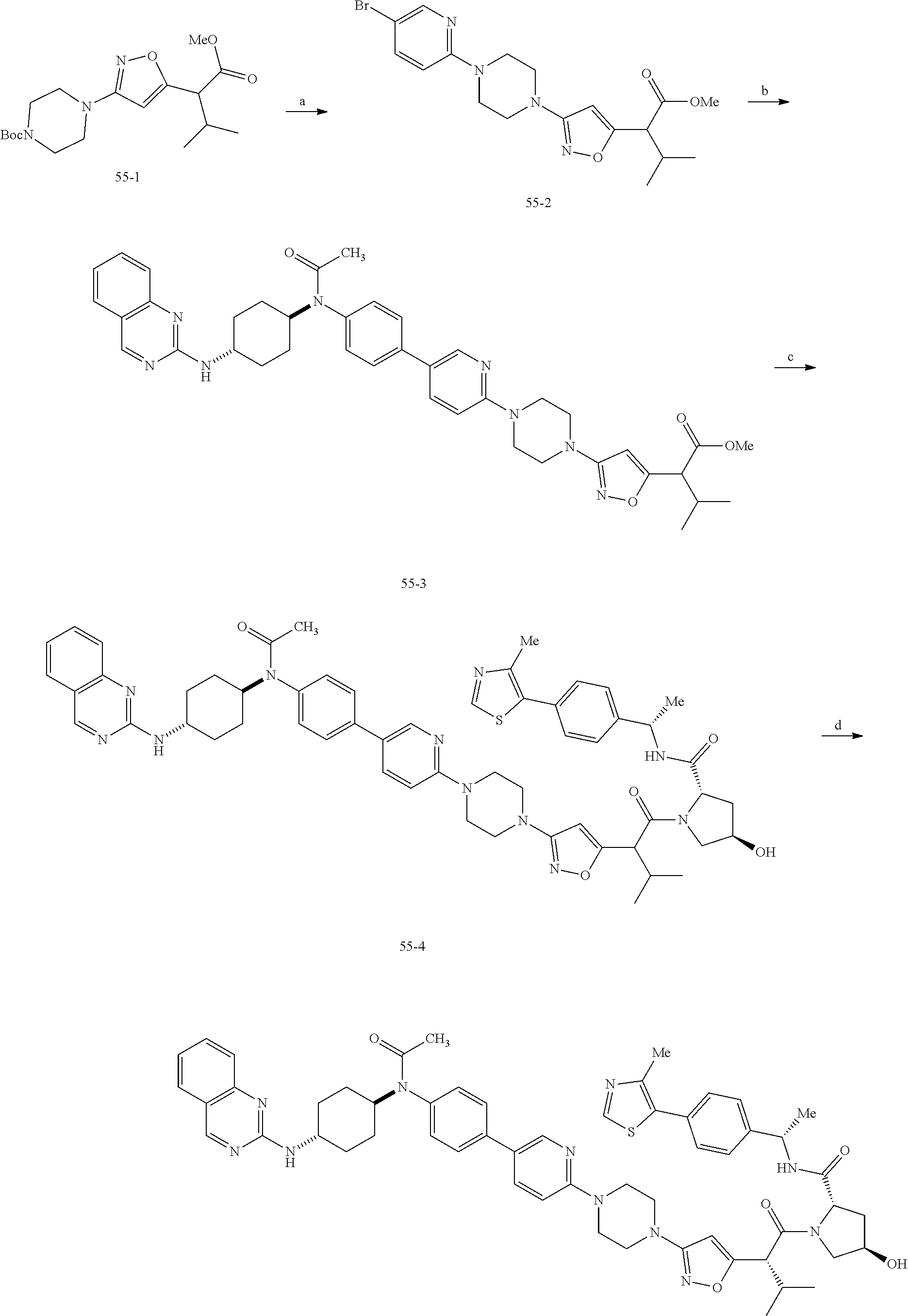

### Synthesis Step a: 55-1

Synthesis Method: The synthesis method for 55-1 can be found in the following reference: J Med Chem. 2024 Jan. 25; 67(2):1262-1313.

### Synthesis Step a: 55-2

Following the method for Intermediate 39, replace the tert-butyl 2-(2-hydroxyethoxy)acetate in the synthesis step with intermediate 55-1 that has been treated with a hydrochloric acid/ethyl acetate solution. ESI-MS: m/z=423.12[M+H]⁺.

### Synthesis Step b: 55-3

Following Synthesis Step 1 for Intermediate 14, replace the 4-Boc-1-(5-bromo-2-pyridyl)piperazine in the synthesis step with intermediate 55-2. ESI-MS: m/z=703.48[M+H]⁺.

### Synthesis Step c: 55-4

Following Synthesis Step e of Example 51, replace intermediate 51-4 with intermediate 55-3. ESI-MS: m/z=1002.12[M+H]⁺.

### Synthesis Step d: Compound 155

Compound 155 is obtained by separation using chiral column chromatography. ESI-MS: m/z=1002.12[M+H]⁺.

### Example 156: (2S,4R)-4-hydroxy-1-((R)-3-methyl-2-(3-(4-(5-(4-(N-((1R,4R)-4-(quinazolin-2-ylamino)cyclohexyl)acetamido)phenyl)pyrimidin-2-yl)piperazin-1-yl)isoxazol-5-yl)butanoyl)-N—((S)-1-(4-(4-methylthiazol-5-yl)phenyl)ethyl)pyrrolidine-2-carboxamide

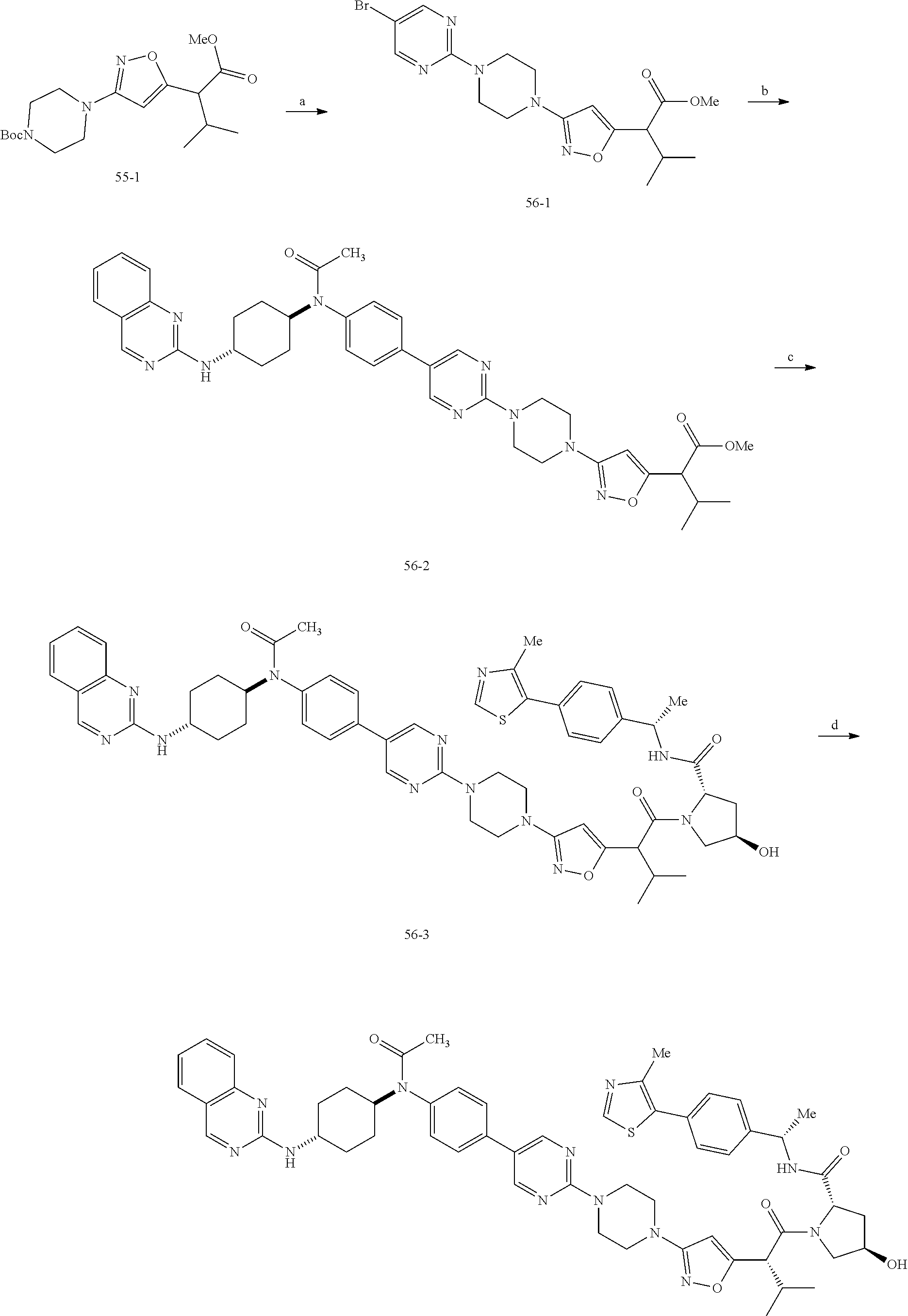

### Synthesis Step a: 56-1

Following the method for Intermediate 40, replace the tert-butyl 2-(2-hydroxyethoxy)acetate in the synthesis step with intermediate 55-1 that has been treated with a hydrochloric acid/ethyl acetate solution. ESI-MS: m/z=424.17[M+H]⁺.

### Synthesis Step b: 56-2

Following Synthesis Step 1 for Intermediate 14, replace the 4-Boc-1-(5-bromo-2-pyridyl)piperazine in the synthesis step with intermediate 56-1. ESI-MS: m/z=704.45[M+H]⁺.

### Synthesis Step c: 56-3

Following Synthesis Step e of Example 151, replace intermediate 51-4 with intermediate 56-2. ESI-MS: m/z=1003.09[M+H]⁺.

### Synthesis Step d: Compound 156

Compound 156 is obtained by separation using chiral column chromatography. ESI-MS: m/z=1003.09[M+H]⁺.

### Example 157

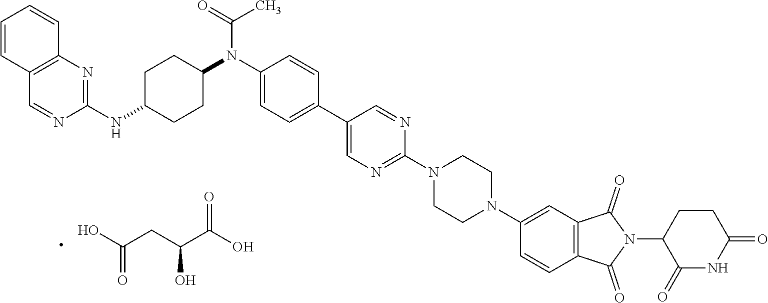

Synthesis Method: In a 25 mL round-bottom flask, 500.0 mg (0.64 mmol) of the compound as shown in Formula 107 and L-malic acid (857.6 mg, 6.4 mmol) are dissolved in 5 mL of anhydrous ethanol. React overnight at room temperature. A white solid precipitates out. Filter with suction to obtain 568 mg of the compound as shown in Formula 157, with a yield of 97%. ESI-MS: m/z=779.70 [M+H]⁺.

### Example 158

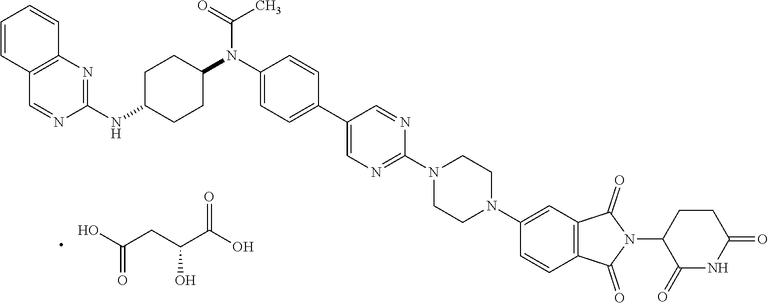

By reference to the preparation method for Formula 157, the starting materials are the compound as shown in Formula 107 and D-malic acid. ESI-MS: m/z=779.70 [M+H]⁺.

### Example 159

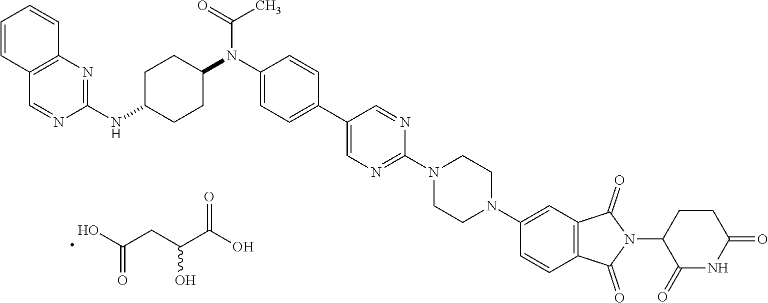

By reference to the preparation method for Formula 157, the starting materials are the compound as shown in Formula 107 and (±)-malic acid. ESI-MS: m/z=779.70 [M+H]⁺.

### Example 160

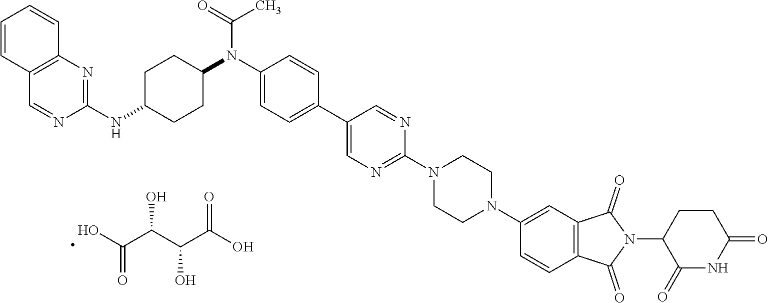

By reference to the preparation method for Formula 157, the starting materials are the compound as shown in Formula 107 and L-tartaric acid. ESI-MS: m/z=779.70 [M+H]⁺.

### Example 161

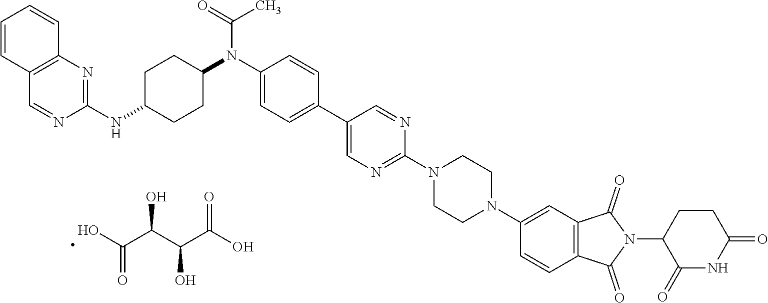

By reference to the preparation method for Formula 157, the starting materials are the compound as shown in Formula 107 and D-tartaric acid. ESI-MS: m/z=779.70 [M+H]⁺.

### Example 162

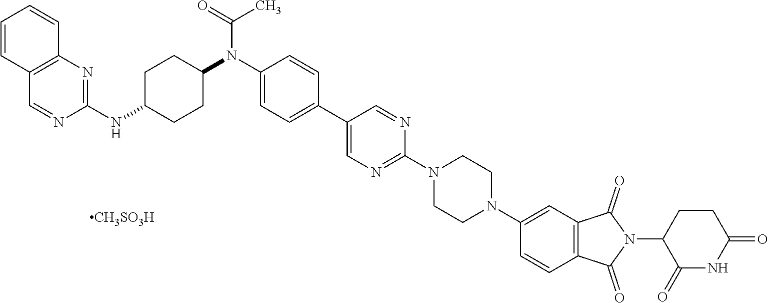

By reference to the preparation method for Formula 157, the starting materials are the compound as shown in Formula 107 and methanesulfonic acid. ESI-MS: m/z=779.70 [M+H]⁺.

### Example 163

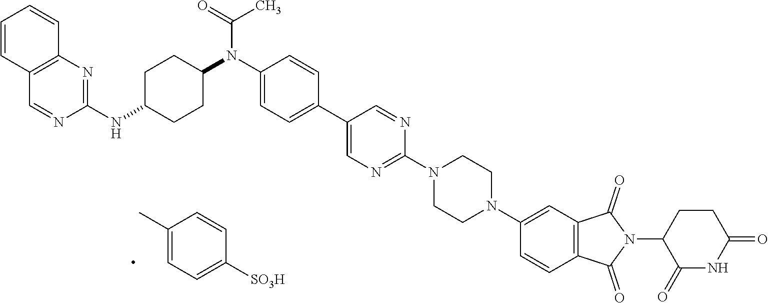

By reference to the preparation method for Formula 157, the starting materials are the compound as shown in Formula 107 and p-toluenesulfonic acid. ESI-MS: m/z=779.70 [M+H]⁺.

### Example 164

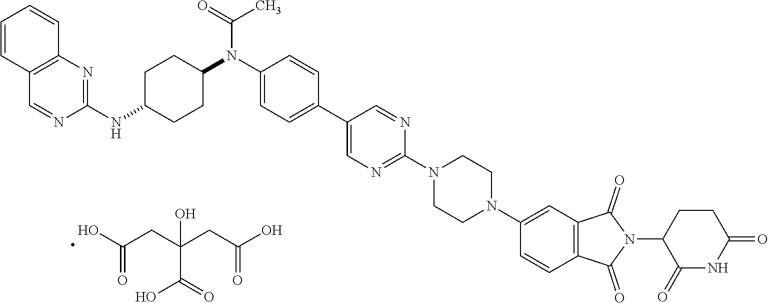

By reference to the preparation method for Formula 157, the starting materials are the compound as shown in Formula 107 and citric acid. ESI-MS: m/z=779.70 [M+H]⁺.

### Example 165

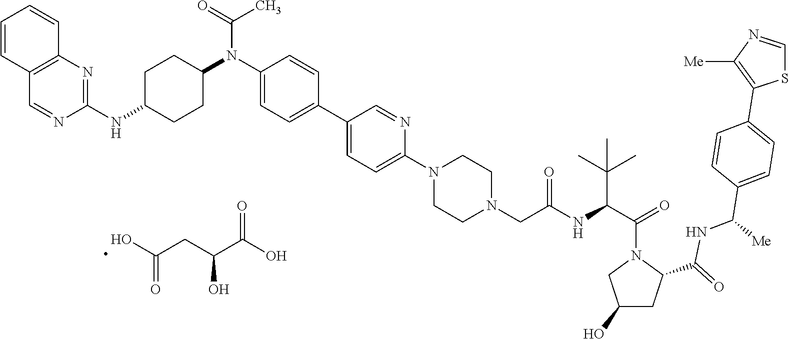

By reference to the preparation method for Formula 157, the starting materials are the compound as shown in Formula 147 and L-malic acid. ESI-MS: m/z~1006.37 [M+H].

### Example 166

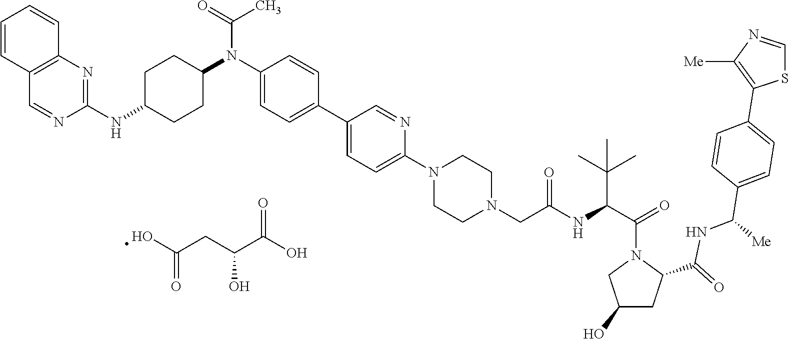

By reference to the preparation method for Formula 157, the starting materials are the compound as shown in Formula 147 and D-malic acid. ESI-MS: m/z=1006.37 [M+H]⁺.

### Example 167

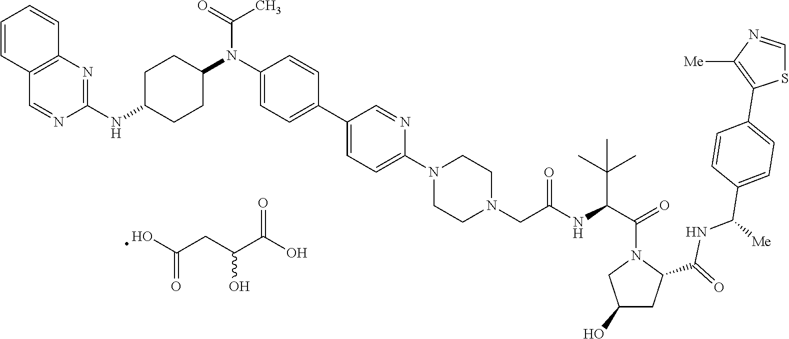

By reference to the preparation method for Formula 157, the starting materials are the compound as shown in Formula 147 and (1)-malic acid. ESI-MS: m/z=1006.37 [M+H].

### Example 168

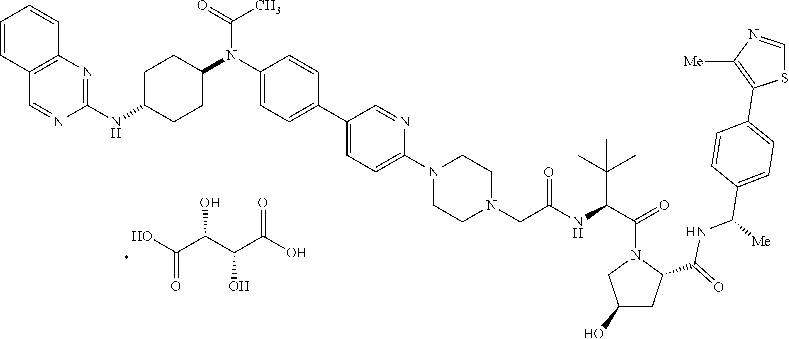

By reference to the preparation method for Formula 157, the starting materials are the compound as shown in Formula 147 and L-tartaric acid. ESI-MS: m/z=1006.37 [M+H]⁺.

### Example 169

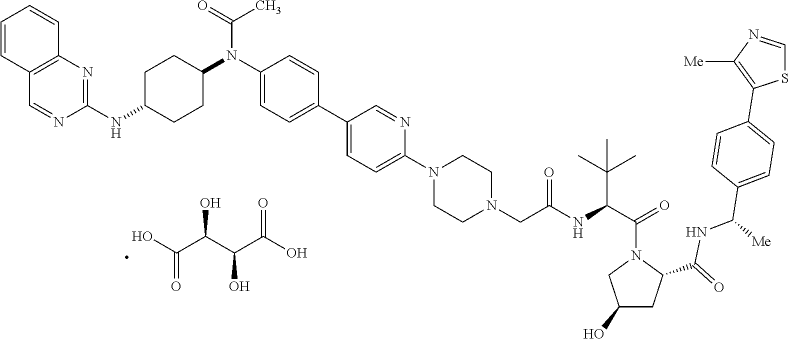

By reference to the preparation method for Formula 157, the starting materials are the compound as shown in Formula 147 and D-tartaric acid. ESI-MS: m/z 1006.37 [M+H]⁺.

### Example 170

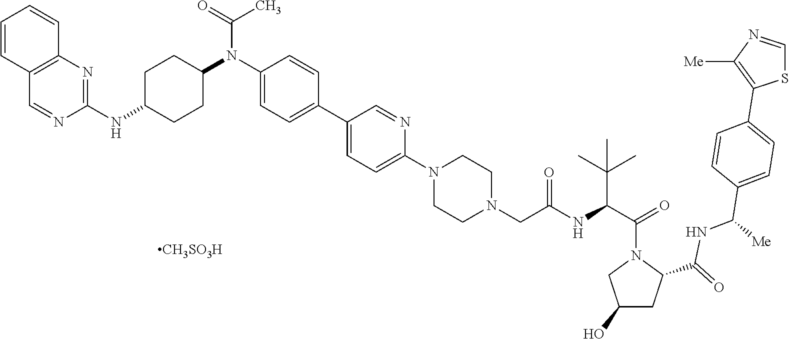

By reference to the preparation method for Formula 157, the starting materials are the compound as shown in Formula 147 and methanesulfonic acid. ESI-MS: m/z=1006.37 [M+H]⁺.

### Example 171

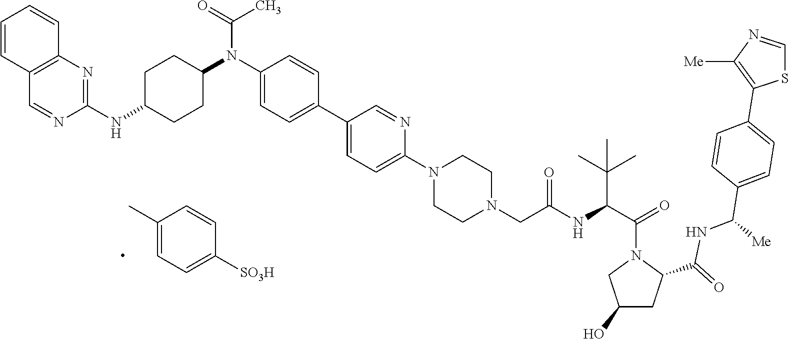

By reference to the preparation method for Formula 157, the starting materials are the compound as shown in Formula 147 and p-toluenesulfonic acid. ESI-MS: m/z=1006.37 [M+H]⁺.

### Example 172

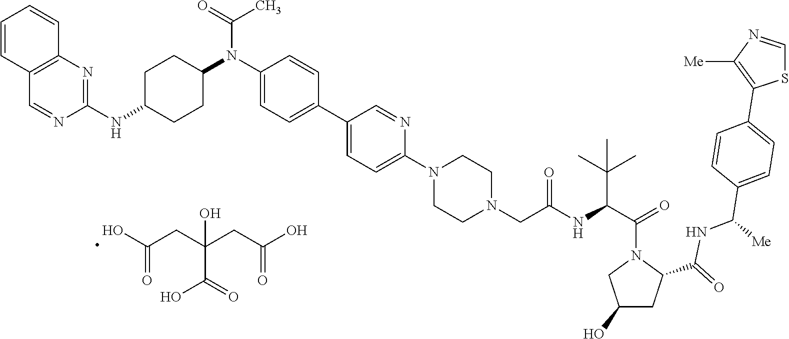

By reference to the preparation method for Formula 157, the starting materials are the compound as shown in Formula 147 and citric acid. ESI-MS: m/z=1006.37 [M+H]⁺.

### Example 3: Biological Activity Test of the Small Molecule Conjugates as Shown in Formulae 101 to 147

### I. Validation of CDK12/13 Degradation Effect and Other Related Protein Degradation Effects via Western Blot

Protein Western blot was used to determine the degradation of CDK12/13 protein in colorectal cancer cells HCT-116, breast cancer cells MDA-MB-231, and leukemia cells Jurkat. The two compounds with the best degradation effects were further validated for their ability to degrade CDK12/13 protein in MDA-MB-231 cells using a concentration gradient. The effect of the compounds on the phosphorylation of serine 2 in the C-terminal domain of RNA polymerase II (CTD-pSer2) was also measured.

### Experimental Materials:

Cell lines: HCT-116, Jurkat, or MDA-MB-231
Culture medium: DMEM/RIPM-1640+10% Hyclone serum
Compound dissolution method: The compounds (small molecule conjugates as shown in Formulae 101-147, MG132) were dissolved in DMSO to prepare a 50 mM stock solution and then diluted to the corresponding concentrations.

### 1. In Vitro Cell Culture and Drug Administration (for HCT-116, Jurkat, or MDA-MB-231)

(1) In vitro cell culture: The selected cells were cultured in a 37 °C constant temperature incubator with 5% CO₂. Once the cell density reached 70-90%, they were passaged for subsequent experiments.

(2) Cell seeding: Cells were digested, and the cell suspension was transferred to a 1.5 mL Eppendorf tube. After centrifugation at 1,000 rpm for 5 min, the cells were resuspended in 2 mL of culture medium and counted. The cell suspension was seeded in a 24-well plate at a density of 200,000 cells per well and allowed to adhere for 8-12 h.

(3) Compound treatment: The 50 mM compound stock solution was diluted with cell culture medium to a final concentration of 0.01 μM-3 μM. Cells were incubated in a 37 °C incubator with 5% CO2 for 3 h-48 h. The DMSO group served as the negative control.

### 2. Cell Lysis

At the end of the treatment time, the cells were collected and washed once with PBS. An appropriate volume of 4% SDS was added to lyse the cells, and the mixture was sonicated until no longer viscous. After centrifugation at 12,000 rpm at room temperature for 30 min, the supernatant was transferred to a new EP tube for protein quantification.

### 3. Protein Quantification

A 2 mg/mL BSA standard was serially diluted by half to create a standard curve at the following concentrations: 2 mg/mL, 1 mg/mL, 0.5 mg/mL, 0.25 mg/mL, 0.125 mg/mL, and 0.0625 mg/mL. A and B solutions from the BCA quantification kit were mixed at a volume ratio of 50:1 (calculating for 200 μL of A solution and 4 μL of B solution per sample). 10 μL of the BSA standards and the samples from step 2 were added to a 96-well plate, followed by 200 μL of the mixed A and B solutions. After gentle mixing, the plate was incubated in the dark at 37 °C for 30 min. Absorbance values were measured at 562 nm, and sample protein concentrations were calculated using the standard curve.

### 4. Western Blot

(1) Protein sample preparation: An amount corresponding to 20 μg of protein from the samples obtained in step 2 was transferred to a new EP tube. 10 μL of 6× Loading Buffer was added, and the total volume was adjusted to 30 μL with distilled water. The samples were denatured by heating at 95 °C for 10 min, and after cooling, they were centrifuged and mixed before the Western blot experiment. The remaining samples were stored at −80 °C.

### (2) Gel Casting:

a. Separating gel: The concentration of the separating gel was chosen based on the molecular weight of the target protein. The gel clamp was first assembled by clamping two glass plates together on a flat surface. The separating gel reagents were added sequentially to a centrifuge tube and mixed by vortexing. The mixture was then poured between the glass plates, and an appropriate amount of isopropanol was added to the top of the gel. The gel was allowed to solidify.

b. Stacking gel: The isopropanol was discarded from the top of the separating gel, and the surface was washed with triple-distilled water. The stacking gel reagents were prepared and mixed by vortexing. The mixture was then poured between the glass plates, a comb was inserted, and the gel was allowed to solidify.

### (3) Electrophoresis:

a. Preparation: The prepared gel clamp was installed in the electrophoresis tank and placed in the electrophoresis apparatus. A diluted 1× Tricine buffer was added to the inner tank, and a 1× Running buffer was added to the outer tank. The setup was left to stand for several minutes.

b. Sample loading: The samples from step 4 (1) were mixed by vortexing, and a certain amount of each sample (5-10 μL) was loaded into the wells. After loading, the electrophoresis apparatus was turned on. The initial setting was a constant voltage of 70 V for 10 min. After confirming the current was normal and the samples had entered the separating gel, the voltage was adjusted to 130 V, and electrophoresis was stopped once the samples had run to the appropriate position.

(4) Sample loading: The samples from step 4 (1) were mixed by vortexing, and a certain amount of each sample (5-10 μL) was loaded into the wells. After loading, the electrophoresis apparatus was turned on. The initial setting was a constant voltage of 70 V for 10 min. After confirming the current was normal and the samples had entered the separating gel, the voltage was adjusted to 130 V, and electrophoresis was stopped once the samples had run to the appropriate position.

a. Transfer: In the transfer clamp, a sponge, a thick filter paper, and a thin filter paper were added in sequence. The gel was then carefully removed from the clamp using a gel spatula to cut away the stacking gel and placed in the center of the filter paper. A PVDF membrane, pre-activated with methanol, was placed on the gel from one side, and air bubbles were removed. A thin filter paper, a thick filter paper, and a sponge were then added in sequence, and the clamp was secured. The transfer assembly was placed in the transfer tank. An appropriate amount of transfer buffer was added to the tank, and ice packs were placed in the outer layer of the tank to prevent heat from affecting the transfer.

b. Blocking: After transfer, the clamp was opened, and the gel and PVDF membrane were removed (if the transfer was successful, the protein ladder would be entirely transferred to the PVDF membrane). The PVDF membrane was then cut according to the molecular weight of the target protein, placed in 5% skim milk, and blocked at room temperature for 60 min.

### (5) Antibody Incubation:

a. Primary antibody incubation: After blocking, the milk was discarded, and the PVDF membrane was washed 3 times with TBST (15 min, 5 min, 5 min). An appropriate amount of primary antibody was added (typically diluted 1:1000 with antibody diluent) and incubated overnight on a shaker at 4 °C.

b. Secondary antibody incubation: The primary antibody was recovered, and the membrane was washed 3 times with TBST (15 min, 5 min, 5 min). Secondary antibody was then added (typically prepared at a 1:5000 dilution in 5% skim milk) and incubated at room temperature for 60 min on a horizontal shaker.

### (6) Exposure:

a. Washing: The secondary antibody was discarded, and the membrane was washed 3 times with TBST (15 min, 5 min, 5 min).

b. Exposure: The ECL detection reagent was prepared by mixing solution A and solution B at a 1:1 ratio. The residual TBST on the membrane was blotted with filter paper, and the membrane was transferred to an exposure plate with forceps. The ECL detection solution was dropped onto the membrane to cover it, and it was left to stand for about 1 min. The plate was then placed in an AI800 exposure machine, and an appropriate exposure time was selected.

### Degradation Rate Calculation Method:

The exposure results were quantitatively analyzed using Image J software. First, the grayscale values of the bands were calculated using Image J. Then, the grayscale value of the target protein was divided by the grayscale value of the internal reference protein for normalization. The calculation formula is:

$${D}_{experimental} = \{ 1 - [ ( {A}_{experimental , target} / {A}_{experimental , internal} ) \div ( {A}_{negative control , target} / {A}_{negative control , internal} ) ] \} \times 100 \%$$

Where A is the grayscale value and D is the degradation rate.

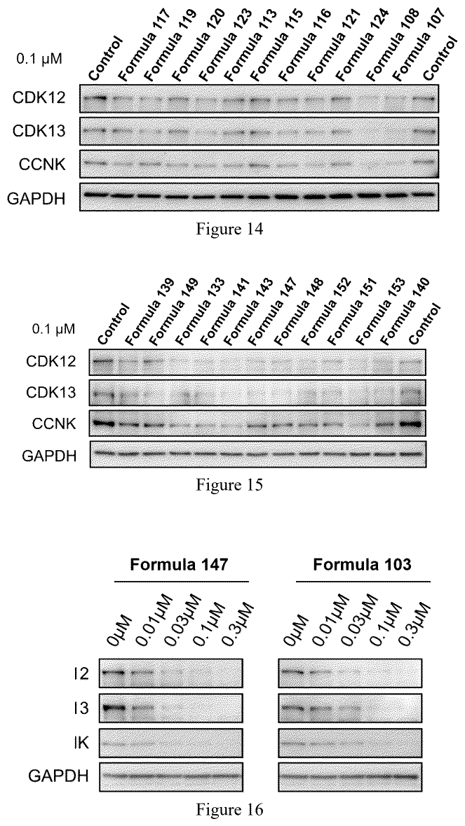

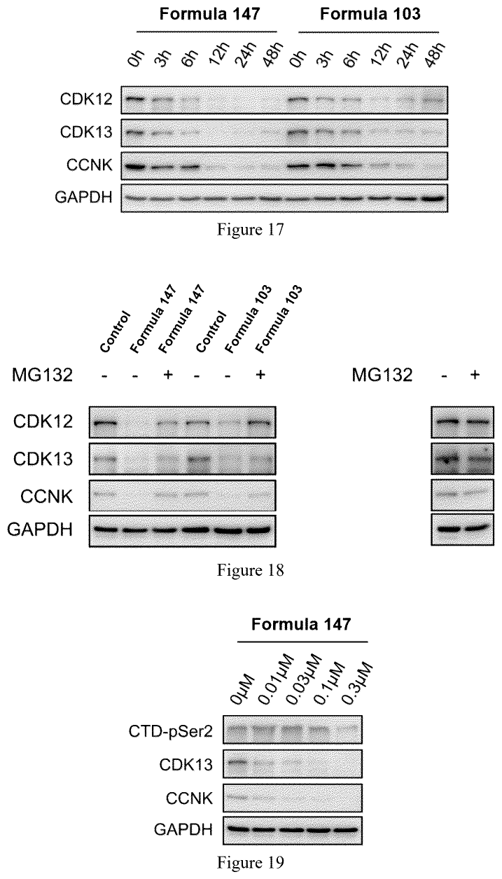

The degradation activity of the small molecule conjugates of the present invention on CDK12 and CDK13 is shown in FIGS. 13-19. In the figures, CDK12, CDK13, and CCNK are the target proteins under study, and β-Actin and GAPDH are the internal reference proteins. Internal reference proteins are used to indicate the total protein amount in each experimental group for quantitative analysis.

As can be seen from the test results in FIGS. 13-17, most of the conjugates can degrade CDK12/13 at a certain concentration. The degradation ability of Formulae 103 and 147 is shown in Table 4.

As can be seen from the test results in FIG. 18, MG132 (a proteasome inhibitor) can reverse the degradation of CDK12/13 and CCNK by Formulae 147 and 103, indicating that the degradation of CDK12/13 and CCNK by Formulae 147 and 103 is via the proteasome pathway, which is consistent with the PROTAC mechanism of action.

As can be seen from the test results in FIG. 19, Formula 47 can dose-dependently downregulate the phosphorylation of serine 2 in the C-terminal domain of RNA polymerase II (CTD-pSer2).

TABLE 4
Degradation activity of the compounds as shown in
Formulae 103 and 147 on CDK12 and CDK13

| | | Formula 103 | | Formula 147 | |
|---|---|---|---|---|---|
| | | DC₅₀ | | DC₅₀ | |
| Cell line name | Cell type | CDK12 | CDK13 | CDK12 | CDK13 |
| MDA-MB-231 | Breast cancer cells | 16 nM | 30 nM | 15 nM | 9 nM |
| HCT116 | Colorectal | 22 nM | 19 nM | 13 nM | 15 nM |
| | cancer cells | | | | |
| KASUMI-1 | Leukemia cells | 15 nM | 18 nM | 17 nM | 10 nM |

The names and activity of the example compounds can be seen in Table 5 below. In the table, +represents the potency of degradation, −represents no degradation observed, and N.D. represents not determined.

TABLE 5
Names and Activity of Example Compounds

| | CDK12 | CDK13 |
|---|---|---|
| | degradation | degradation |
| Formula | Activity | Activity |
| 101 | + + | + + |
| 102 | + + + | + + + + |
| 103 | + + + + + | + + + + + |
| 104 | + | + |
| 105 | N.D. | N.D. |
| 106 | + + + | + + + |
| 107 | + + + + + | + + + + + |
| 108 | + + + + | + + + + |
| 109 | + + + + | + + + + |
| 110 | + + | + + + |
| 111 | + + | + + |
| 112 | + + + + | + + + + |
| 113 | + | + |
| 114 | + + | + + |
| 115 | + + | + + |
| 116 | + | + |
| 117 | + + | + + + |
| 118 | + + | + + + |
| 119 | + + | N.D. |
| 120 | + | + |
| 121 | N.D. | N.D. |
| 122 | N.D. | N.D. |
| 123 | + + + + | + + + |
| 124 | + + | + + |
| 125 | + + | + |
| 126 | + + | + |
| 127 | + + + | + + + |
| 128 | + | + |
| 129 | + | + |
| 130 | + + | + + |
| 131 | + + + | + + + |
| 132 | + + + | + + + |
| 133 | + + + | + + + + |
| 134 | + + + | + + + |
| 135 | + + + | + + |
| 136 | + + + | + + + |
| 137 | + | + |
| 138 | + + | − |
| 139 | + + | + + |
| 140 | + + | + + + |
| 141 | + + + + + | + + + + |
| 142 | + + + + + | + + + + + |
| 143 | + + + + | + + + + |
| 144 | + + + + + | + + + + |
| 145 | − | + |
| 146 | + | + |
| 147 | + + + + + | + + + + + |
| 148 | + + + + + | + + + + + |
| 149 | + + + | + + |
| 150 | N.D. | N.D. |
| 151 | + + + | + + + + |
| 152 | + + + + | + + + + |
| 153 | + + + + | + + + + + |
| 154 | + + + + | + + + + + |
| 155 | + + + + + | + + + + + |
| 156 | + + + + + | + + + + + |
| 158 | + + + + + | + + + + + |
| 167 | + + + + + | + + + + + |

+ represents the degradation effect on the protein, and more + symbols indicate a more significant degradation.
− represents no degradation of the protein.
Specifically:
+ + + + + represents DC₅₀ < 100 nM
+ + + + represents 100 nM < DC₅₀ < 0.5 μM
+ + + represents 0.5 μM < DC₅₀ < 1 μM
+ + represents 1 μM < DC₅₀ < 3 μM
+ represents 3 μM < DC₅₀ < 10 μM
− represents no degradation activity

### II. Staining-Based Detection of the Proliferation Inhibitory Effect of Compounds on Tumor Cells

The proliferation inhibition of compounds on leukemia cell lines MV4-11 and KASUMI-1 was determined using the CCK8 assay, while the inhibition on solid tumor cell lines breast cancer cells BT-549, MDA-MB-231, pancreatic cancer cells PANC1, ovarian cancer cells OVCAR8, and colorectal cancer cells HCT-116, HCT-15 was determined using the Sulforhodamine B (SRB) assay.

CCK8 Assay: First, MV4-11 and KASUMI-1 cells were seeded in a 96-well plate at a density of 5,000 cells per well. Then, a concentration gradient (0.03 μM-10 μM) of the compounds was administered for 72 h, with DMSO solvent serving as the negative control. At the end of the treatment, 10 μL of CCK8 reagent was added to each well. The plate was incubated for 4 hours at 37 °C in the dark. After mixing, the absorbance value was measured at 450 nm.

SRB Assay: First, MDA-MB-231, L-02, and HK-2 cells were seeded in a 96-well plate at a density of 3,000 cells per well. After 8-12 h of cell adhesion, a concentration gradient (62.5 nM-10 μM) of the compounds was administered for 72 h, with DMSO solvent serving as the negative control. At the end of the treatment, the cells were washed once with 70 μL of PBS, then fixed with 70 μL of 10% trichloroacetic acid at 4 °C for 1 h. After rinsing with tap water and drying in an oven, 70 μL of SRB staining solution was added to each well for 20 min at room temperature. The staining solution was then discarded, and the cells were rinsed with 1% glacial acetic acid and dried in an oven. Finally, 150 μL of 10 mM Tris-base dissolving solution was added, and the plate was shaken for 15 min before the absorbance value was measured at 540 nm.

Methodology for Combination Drug Use: MD-MB-231 cells were seeded in a 6-well plate at a density of 2,000 cells per well. After 24 h of cell adhesion, the cells were treated with a specific concentration (0.5 μM, 1 μM) of the PARP inhibitor Olaparib in combination with a specific concentration (0.5 μM, 1 μM) of the compound of Formula 147 for 10 days. The solvent DMSO served as the negative control. At the end of the treatment, the cells were washed once with 100 μL of PBS, then fixed with 700 μL of 10% trichloroacetic acid at 4 °C for 1 h. After rinsing with tap water and drying in an oven, 70 μL of SRB staining solution was added for 20 min at room temperature. The staining solution was discarded, and the cells were rinsed with 1% glacial acetic acid and dried in an oven. The plate was then photographed, and the number of cell clones in each group was counted using Image J software.

The calculation formula for cell proliferation inhibition rate from CCK8 and SRB assays is:

Rₑₓₚₑᵣᵢₘₑₙₜₐₗ=(1−ODₑₓₚₑᵣᵢₘₑₙₜₐₗ/OD_{negative control})×100%, where OD is the absorbance value and R is the cell proliferation inhibition rate.

The calculation formula for cell proliferation inhibition rate from combination drug use is:

Rₑₓₚₑᵣᵢₘₑₙₜₐₗ=(1−Kₑₓₚₑᵣᵢₘₑₙₜₐₗ/K_{negative control})×100%, where K is the cell clone count and R is the cell proliferation inhibition rate.

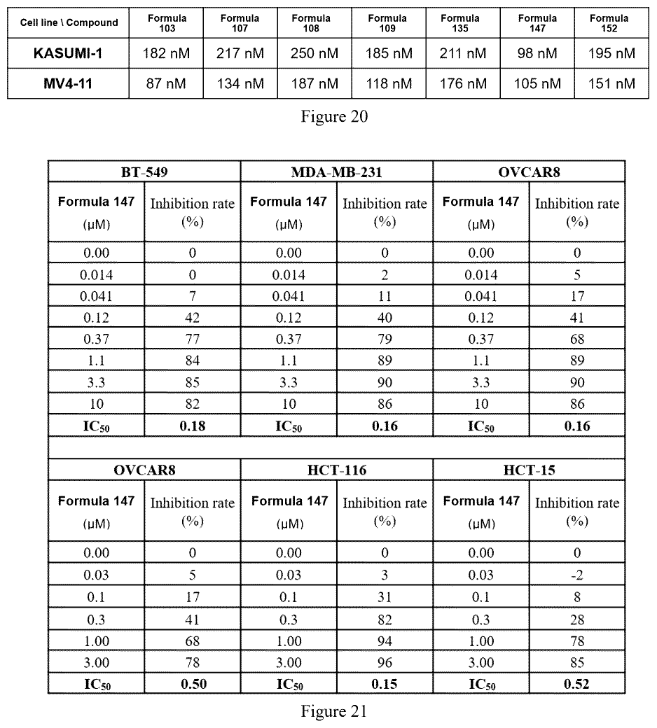

The results are shown in FIGS. 20, 21 and 22.

As shown in FIG. 20, the small molecule conjugates provided in this patent application efficiently inhibit the proliferation of leukemia cells KASUMI-1 and MV4-11.

As shown in FIG. 21, the small molecule conjugates provided in this patent application efficiently inhibit the proliferation of various tumor cells such as breast cancer, colorectal cancer, cervical cancer, and pancreatic cancer cells.

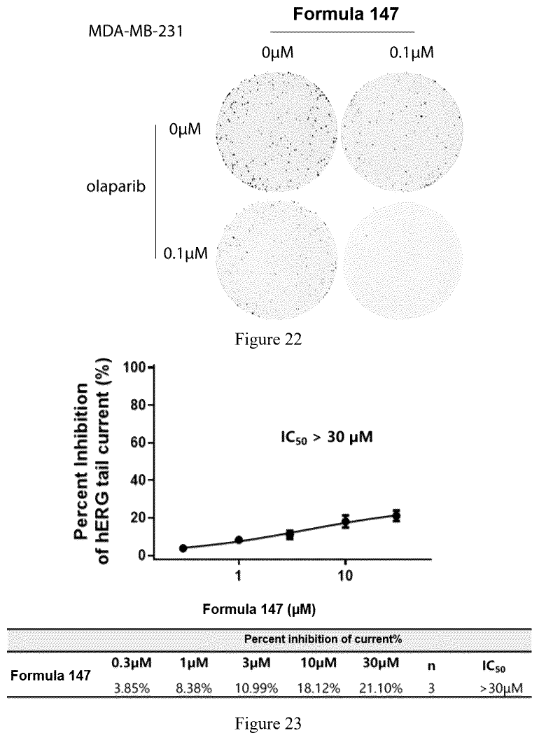

As shown in FIG. 22, the combination therapy of the small molecule conjugate described in this patent application and a PARP inhibitor can effectively and synergistically enhance the proliferation inhibitory activity on breast cancer cells MDA-MB-231, demonstrating a significant additive and complementary effect with existing clinical drugs.

III. Manual Patch-Clamp Detection of the Effect of Compounds on hERG Channel Currents Stably Expressed in HEK293 Cells

### (1) Sample Preparation

A 30 mM stock solution of compound 147 was prepared in DMSO. The stock solution was serially diluted with DMSO to prepare secondary stock solutions of 10 mM, 3 mM, 1 mM, and 0.3 mM. 10 μL of the secondary stock solution was diluted in 10 mL of extracellular solution to prepare final concentrations of 30 μM, 10 μM, 3 μM, 1 μM, and 0.3 μM for electrophysiological detection. A 10 mM stock solution of the positive control, cisapride, was prepared in DMSO. The stock solution was serially diluted with DMSO to prepare secondary stock solutions of 1000 μM, 100 μM, 10 μM, 1 μM, and 0.1 μM. 10 μL of the secondary stock solution was diluted in 10 mL of extracellular solution to prepare final concentrations of 1000 nM, 100 nM, 10 nM, 1 nM, and 0.1 nM for electrophysiological detection.

### (2) Cell Preparation

Before the electrophysiological experiment, the hERG cell line should be maintained in the logarithmic growth phase at a maximum density of 70%. All reagents were preheated to 37 °C before use.
a. Discard the old medium from the 6 cm culture dish.
b. Add 1 mL of PBS, gently shake the culture dish to rinse the bottom, then discard.
c. Add 1 mL of trypsin, gently shake the culture dish to cover all the cells.
d. Incubate at 37 °C for 2-3 minutes, gently pipette to suspend the adherent cells.
e. Transfer the cell suspension into a sterile centrifuge tube, centrifuge at 1000 rpm/min for 5 min, and adjust the cell concentration to 2×103/mL. 300 μL of the cell solution was seeded in a 24-well plate on cell slides. After the cells adhered well, the patch-clamp detection experiment was performed.

### (3) Testing

hERG tail current data was collected and stored on a computer using Patchmaster software via an EPC-10 amplifier.

a. The cell slide was removed from the culture dish with tweezers, extracellular solution was added, and it was placed in a bath on the stage of an inverted microscope.

b. Glass microcapillaries were pulled using a P-1000 microelectrode puller. The intracellular solution was used to fill one-third of the volume of the glass microcapillary (recording electrode), which was then placed in the electrode holder.

c. A motorized micromanipulator (Scientifica-Double 1U) was used to bring the recording electrode into contact with the cell surface. At this point, due to a sudden increase in electrode resistance, the current value represented by the sealing test pulse displayed in the membrane test window decreases. Positive pressure was removed, and a negative pressure of 0.5 cm H₂O was applied. The sealing resistance was observed to increase rapidly until a Giga-seal was achieved. After the seal resistance between the recording electrode and the cell membrane was greater than 1 GΩ, a negative pressure was applied to rupture the membrane, forming a whole-cell recording mode. After the rupture was stable, the membrane capacitance (Cs) and series resistance (Rs) were compensated.

d. Stimulation protocol: The holding potential was clamped at −80 mV, depolarized to 40 mV for a duration of 4000 ms, and then repolarized to −50 mV for 4000 ms, which elicited the tail current. The current was recorded once every 15 s.

e. The hERG potassium channel current was recorded at room temperature before drug addition. After the control current reached a steady state (i.e., the most recent four consecutive current traces overlapped), the drug was added using a cumulative dosing method, starting with the negative control (0.1% DMSO), followed by five drug concentrations in sequence.

### (4) Data Analysis

The original data hERG tail current peak values were extracted from the PatchMaster software. The current inhibition rate was calculated using the following formula:

$$Peak tail current inhibition rate = ( 1 - ( Peak tail current compound ) / ( Peak tail current vehicle ) )$$

The mean and standard error were calculated for each concentration. The concentration-effect relationship was fitted to the Hill equation:
I=Imax·{1/[1+(C_{1/2}/[C])^{h}]}, Where [C] represents the drug concentration, C_{1/2} is the half-maximal inhibitory concentration (IC₅₀), and h is the Hill coefficient. The analysis and statistics were performed using Graphpad Prism 8.0.2 software.

The results are shown in FIG. 23.

As shown in FIG. 23, the small molecule conjugate Formula 147 provided in this patent application has a weak or no inhibitory effect on the hERG channel, suggesting that Formula 147 will not cause arrhythmia.

### IV. In Vivo Pharmacokinetics in Mice

### (1) Sample Preparation

10 mg of compound 147 was dissolved in 0.5 mL of ethanol solution to make a stock solution. This was then diluted with normal saline to a total volume of 10 mL.

### (2) Intraperitoneal Administration

8 nude mice (4 female, 4 male) were administered the compound at a dose of 30 mg/kg via oral gavage.

### (3) Sample Collection and Processing

Blood was collected from the orbital sinus before administration and at 15 min, 30 min, 1 h, 3 h, 6 h, 8 h, and 12 h after administration. The blood volume collected from each animal did not exceed 0.1 mL per collection. Plasma samples were collected, and 3 volumes of acetonitrile containing 0.20 g/mL internal standard were added to precipitate proteins. The mixture was vortexed for 10 min and centrifuged at 10000 r/min for 10 min. The supernatant was collected.

### (4) Data Acquisition and Analysis

Samples from each time point were injected for LC-MS-MS analysis. The obtained concentration-time data were calculated using DAS3.0 pharmacokinetic software with the statistical moment method to obtain key absorption kinetic parameters such as T_{1/2}, AUC₍₀₋ₜ₎, Cₘₐₓ, Tₘₐₓ, and other pharmacokinetic parameters.

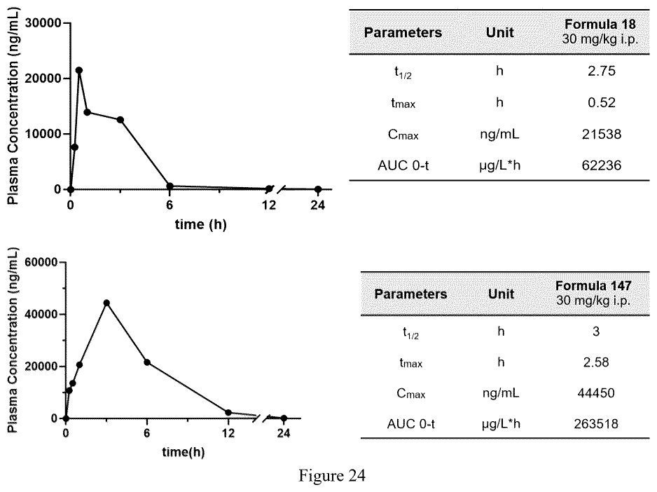

The results are shown in FIG. 24.

As shown in FIG. 24, the small molecule conjugate Formula 147 provided in this patent application exhibits good pharmacokinetic properties in mice when administered at a dose of 30 mg/kg via intraperitoneal injection. The area under the drug concentration-time curve (AUC) is 263518 μg/mL*h, and the half-life is 2.58 hours.

The present invention discloses a small molecule conjugate for degrading CDK12/13, a preparation method, and its applications. Its structure is: T-L-E. In Formula I, T represents the core molecule, which is a ligand for CDK12/13 protein; L represents the connecting part between T and E, also known as a Linker; and E represents a ligand for an E3 ligase. The compound as shown in Formula I can be synthesized through an amide condensation reaction, a substitution reaction, or a click reaction, etc., between a T-part derivative and an E3 enzyme ligand and its derivatives. This compound demonstrates high-efficiency CDK12/13 degradation activity, highly specific tumor cell proliferation inhibitory activity, and efficient therapeutic effects in combination with other anti-tumor drugs in various cell lines. From a biological and therapeutic perspective, it can be used as a lead compound for the treatment of various cancers and other CDK12/13 abnormal diseases.

What is claimed:

1. A small molecule conjugate, characterized in that it has a structure represented by Formula I, or a pharmaceutically acceptable salt, cocrystal, tautomer, stereoisomer, mixture of stereoisomers, solvate, hydrate, polymorph, isotope-enriched derivative or prodrug thereof: 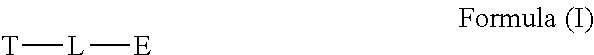
In Formula (I), T represents a ligand for CDK12/13 protein, E represents a ligand for E3 ligase, and L represents a linker chain connecting T and E;
The T is selected from the groups represented by formula (II) or (II′): 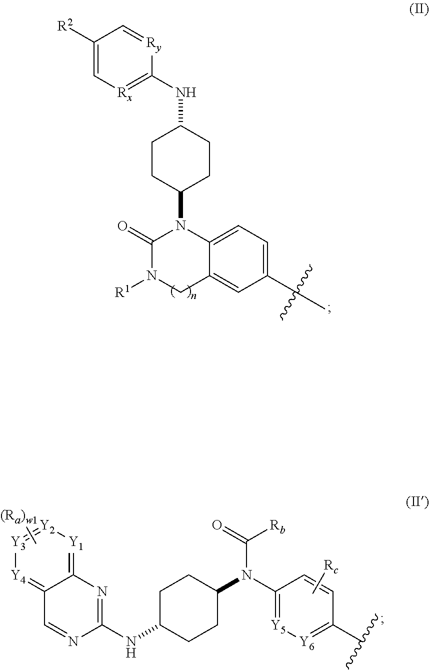
In Formula (II):
R¹ is selected from hydrogen, N-substituted amide, C1-C4 alkyl-substituted acyl, C1-C6 alkyl, C1-C6 alkenyl, C1-C6 alkynyl, 3-8 membered carbocyclic group, 3-8 membered heterocyclic group, 5-10 membered aryl, 5-10 membered heteroaryl, or a nitrogen protecting group; or optionally, when there are two R¹, the two R¹ and the atom (N) between them together form a substituted or unsubstituted heterocyclic group or a substituted or unsubstituted heteroaryl; the nitrogen protecting groups include tert-butoxycarbonyl, benzyloxycarbonyl, allyloxycarbonyl, and p-methoxybenzyl;
R² is selected from hydrogen, N-substituted amide, C1-C4 alkyl-substituted acyl, C1-C6 alkyl, C1-C6 alkenyl, C1-C6 alkynyl, 3-8 membered carbocyclic group, 3-8 membered heterocyclic group, 5-10 membered aryl, 5-10 membered heteroaryl, —CN, —ORD1, —N(R^{D1a})₂, or —SRD1; wherein R^{D1} is hydrogen, N-substituted amide, C1-C4 alkyl-substituted acyl, C1-C6 alkyl, C1-C6 alkenyl, C1-C6 alkynyl, 3-8 membered carbocyclic group, 3-8 membered heterocyclic group, 5-10 membered aryl, 5-10 membered heteroaryl, an oxygen protecting group when connected to an oxygen atom, or a sulfur protecting group when connected to a sulfur atom; wherein, the oxygen protecting group is selected from dihydropyran, methoxymethyl, benzyl, methoxybenzyl, trimethylsilyl, and tert-butyldimethylsilyl, and the sulfur protecting group is selected from acetamidomethyl, benzylacetamidomethyl, and tert-butylthio;
Wherein the two R^{D1a} are each independently selected from hydrogen, N-substituted amide, C1-C4 alkyl-substituted acyl, C1-C6 alkyl, C1-C6 alkenyl, C1-C6 alkynyl, 3-8 membered carbocyclic group, 3-8 membered heterocyclic group, 5-10 membered aryl, 5-10 membered heteroaryl, or a nitrogen protecting group; or optionally, the two R^{D1a} and the intermediate atom together form a substituted or unsubstituted heterocyclic ring or a substituted or unsubstituted heteroaryl ring;
Rx and Ry in Formula II are each independently selected from CH or N;
n in Formula II is 0, 1, 2, 3, 4, or 5; [symbol]
and [symbol] represents that the two nitrogen atoms (N) attached to the six-membered ring adopt a trans configuration relative to each other;
In Formula (II′):
Y₁, Y₂, Y₃, Y₄, Y₅, and Y₆ are independently selected from CH, C, or N.
Rₐ is selected from C1-C6 alkyl, C1-C6 haloalkyl, C3-C8 cycloalkyl, and C3-C8 heterocyclic group; wherein the alkyl, cycloalkyl, and heterocyclic group can be further substituted by one or more substituents selected from: halogen, C1-C4 alkyl, C1-C4 haloalkyl, C1-C4 alkoxy, cyano, oxo, carboxyl, ester group, amide group, and hydroxyl.
R_{b} is selected from C1-C6 alkyl, C1-C6 haloalkyl, C3-C8 heterocyclic group, C1-C6 alkoxy, C1-C6 alkoxymethylene, benzyl, cyano, NR_{bb}R_{bc}, and hydroxyl; R_{bb} and R_{bc} are each independently selected from H, C1-C6 alkyl, and C1-C6 haloalkyl; wherein the alkyl, alkoxy, and alkoxymethylene can be further substituted by one or more of the following substituents: hydroxyl, halogen, C1-C4 alkyl, C1-C4 haloalkyl, and amino substituted by one or two methyl groups.
R_{c} is selected from H, halogen, C1-C4 alkyl, C1-C4 haloalkyl, C1-C4 alkoxy, cyano, OR_{cc}, oxo, carboxyl, and hydroxyl; R_{cc} is C1-C4 alkyl or C1-C4 haloalkyl. [symbol]
and [symbol] represents that the two nitrogen atoms (N) attached to the six-membered ring adopt a trans configuration relative to each other;
w1 is 0, 1, 2, 3, or 4;
The E is selected from any of the following groups: 3-amino-N-(2,6-dioxo-3-piperidinyl) phthalimide group, 3-(7-amino-3-oxo-1H-isoindol-2-yl)piperidine-2,6-dione group, thalidomide group, (2S,4R)-1-((S)-2-(3-aminopropanamido)-3,3-dimethylbutanoyl)-4-hydroxy-N-(4-(4-methylthiazol-5-yl)benzyl)pyrrolidine-2-carboxamide group, [(4R,5S)-4,5-bis(4-chlorophenyl)-2-[4-(1,1-dimethylethyl)-2-ethoxyphenyl]-4,5-dihydro-4,5-dimethyl-1H-imidazol-1-yl][4-[3-(methylsulfonyl)propyl]-1-piperazinyl]methanone group, and henylglutarimide.

2. (canceled)

3. The small molecule conjugates according to claim 1, characterized in that in Formula (II):
R¹ is selected from ethyl, 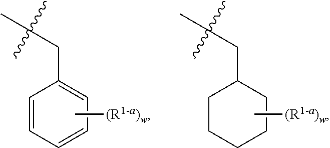
or a protected-amine;
w is 0, 1, 2, 3, 4, 5, or 6;
When there are one or more R^{1-a}, one or more R^{1-a} are each independently selected from halogen, N-substituted amide, C1-C4 alkyl-substituted acyl, and C1-C6 alkyl;
R² is selected from halogen, cyano, and trifluoromethyl;
The nitrogen protecting group includes tert-butoxycarbonyl, benzyloxycarbonyl, allyloxycarbonyl, and p-methoxybenzyl;
In Formula (II′):
R_{b} is selected from methyl, ethyl, propyl, C1-C4 alkyl substituted by one or more fluorine atoms, C1-C4 alkyl substituted by one or more hydroxyl groups, and C3-C8 heterocyclic group;
Rₑ is one or more halogen, C1-C4 alkyl, C1-C4 haloalkyl, or amines substituted by one or two methyl groups.

4. The small molecule conjugates according to claim 1, characterized by the fact that T is one of the following structures:

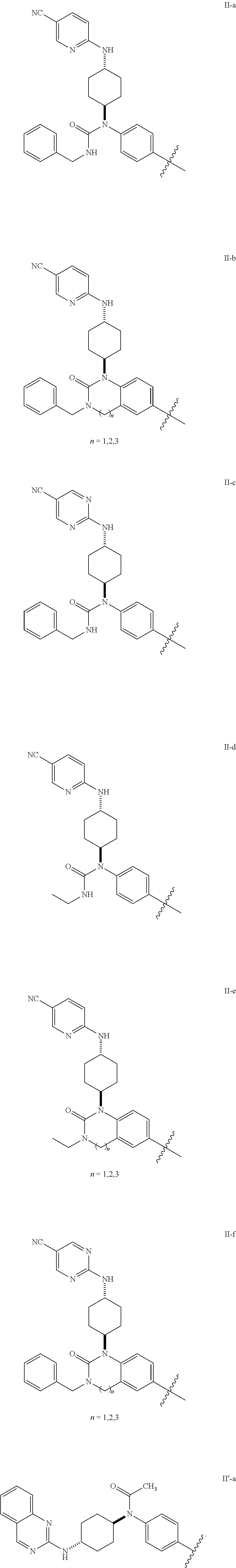

5. (canceled)

6. A small molecule conjugate according to claim 1, wherein:
E is selected from one of the following structures: 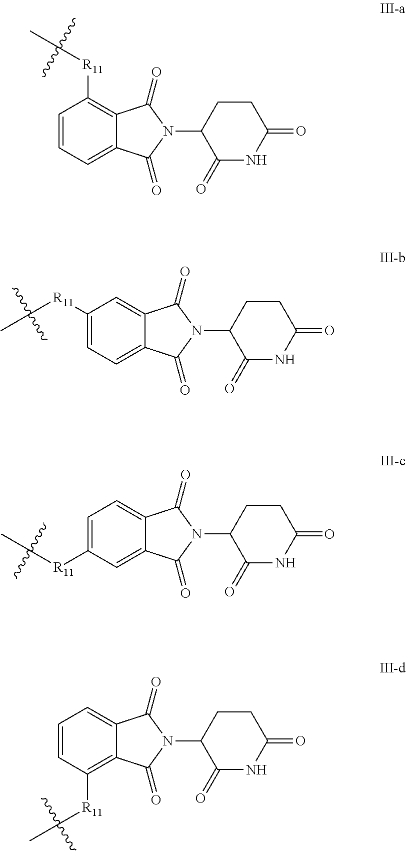 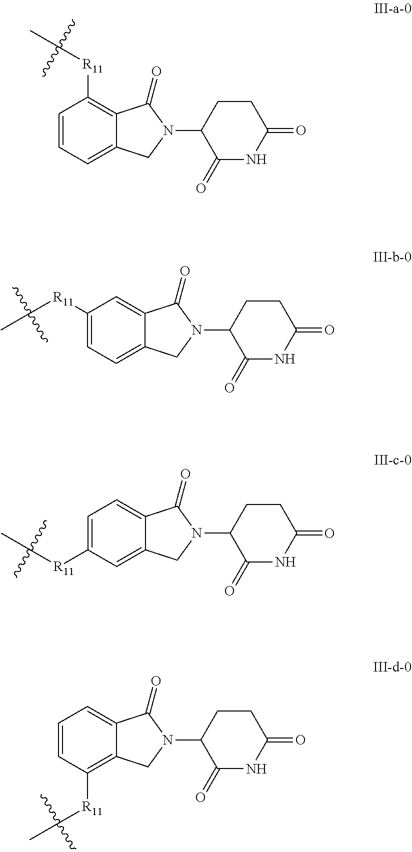 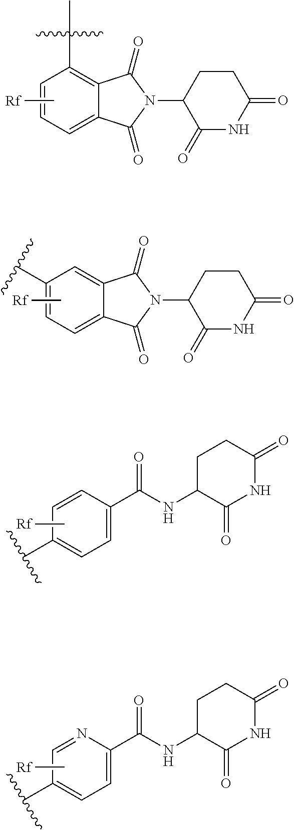 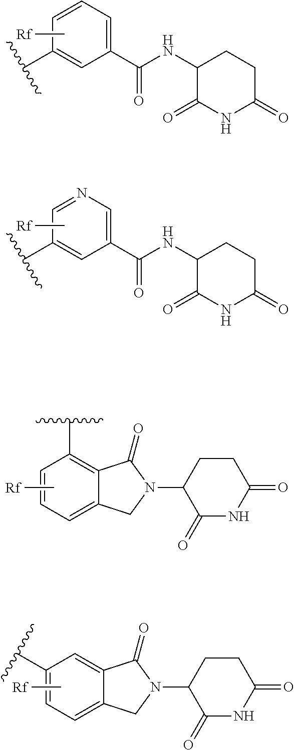 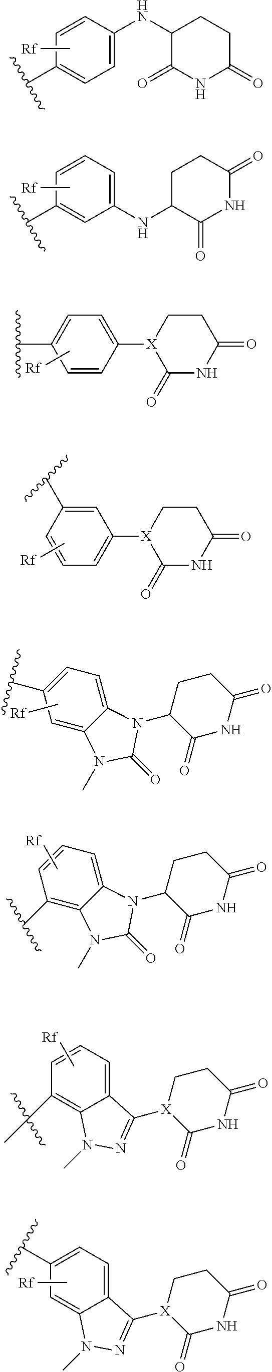
In formulae III-a to III-d and III-a-0 to III-d-0, R1 is NH, O or CH₂;
R_{f} is selected from H, F, Cl, CH₃, OMe, CN, CF₃, CHF₂, CH(CH₃)₂, cyclopropyl, and C(O)NH₂;
X is selected from CH and N;
or, said E is selected from one of the following structures: 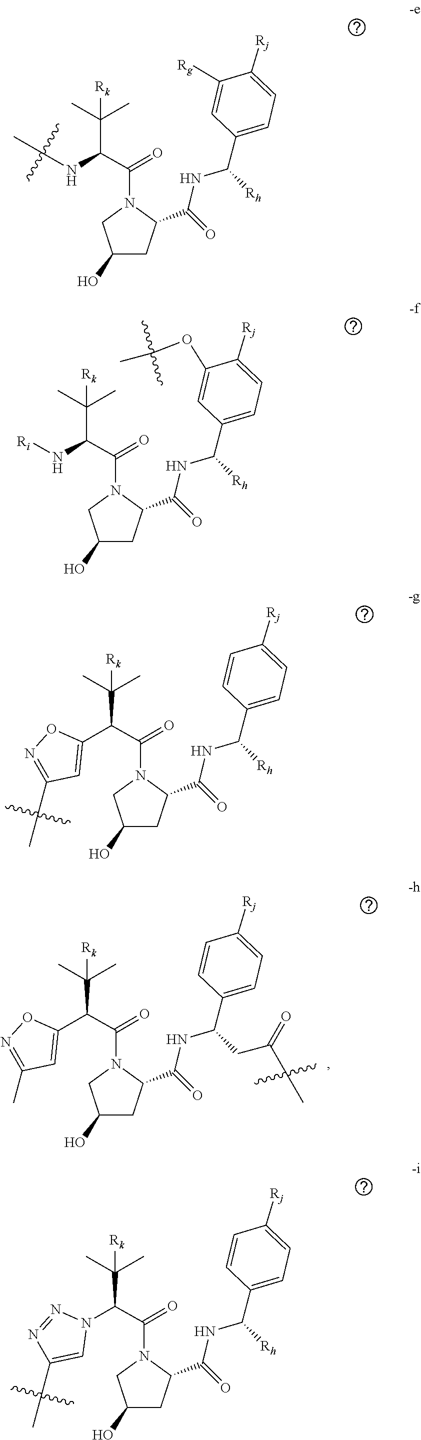 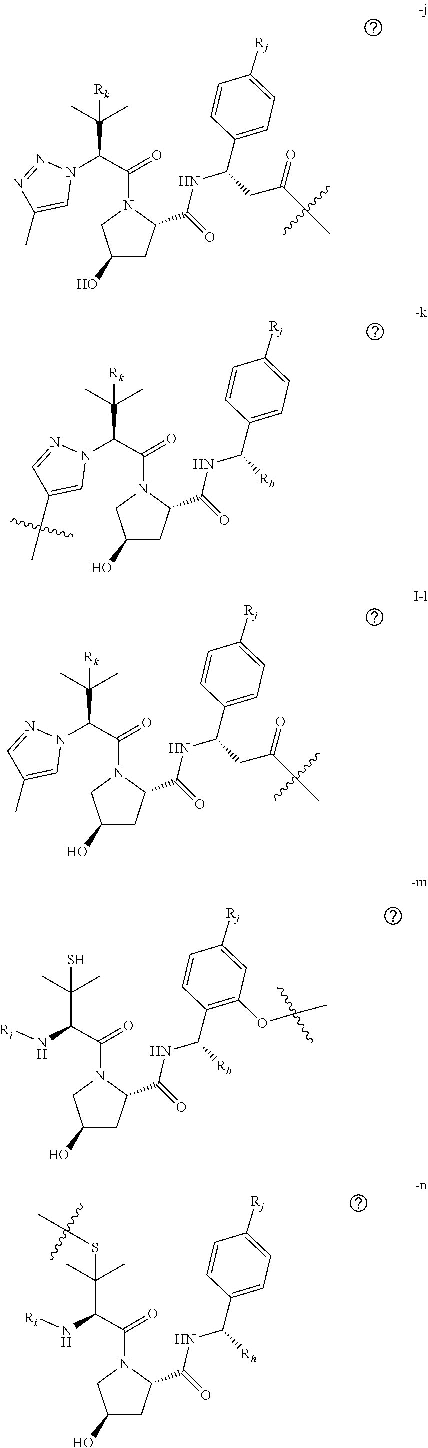 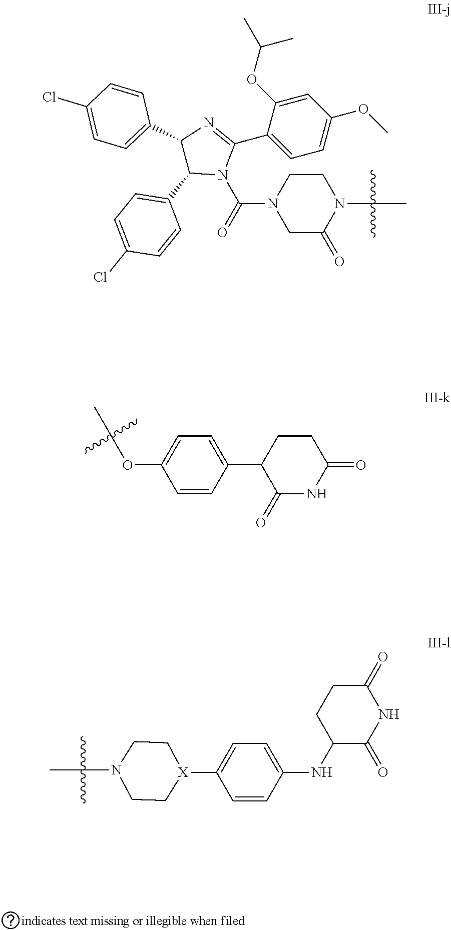
R_{g} is selected from H, OH, Cl, Br, ethynyl, and cyano;
Rₕ is selected from H and CH₃;
Rᵢ is selected from 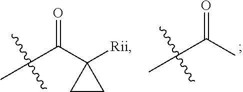
wherein Rᵢᵢ is —CN, —F, —COCH₃;
Rⱼ is selected from: H, OH, CN, F, Cl, Br, or formula: 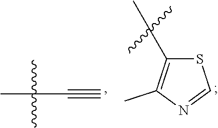
Rₖ is selected from: H, CH₃;
In said formula III-1, X═N or CH.

7. The small molecule conjugate according to any one of claim 1, characterized in that: said L is a chemical group or a chemical bond; when L is a chemical bond, said L is one of the following structural units, or any structural group composed of the following structural units: 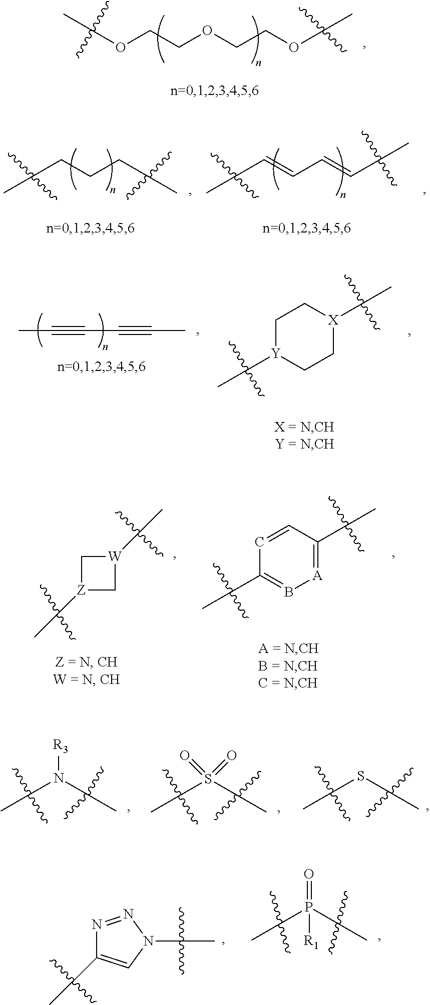
—(CH₂)ₘ— (m=1,2,3,4), —CHR₂—, —NH—, 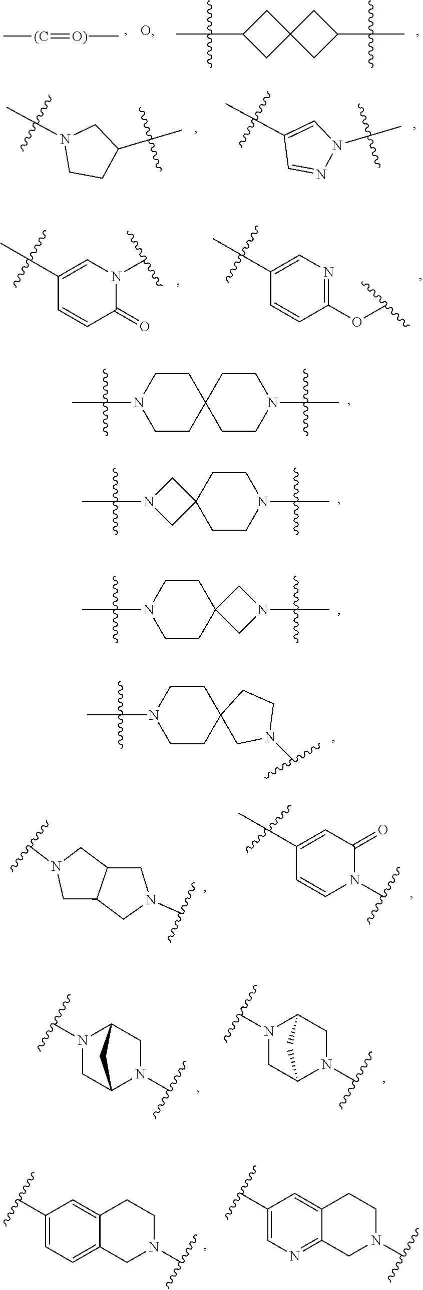
In the said heteroatom-containing unit, R₁ is independently selected from alkyl, alkoxy, or hydrogen;
In the said structural unit, R₂ and R₃ are independently selected from alkyl and alkoxy;
The said alkyl is specifically methyl, ethyl, propyl, isopropyl, n-butyl, or tert-butyl; and the said alkoxy is —OEt or —OMe.

8. The small molecule conjugate according to claim 1, characterized in that: said L is selected from the following structures: 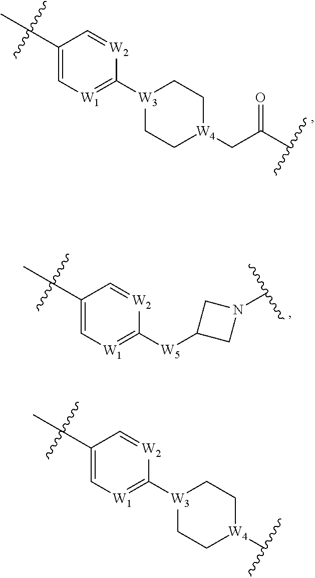
In the above structural formulae, said W₁, W₂, W₃, and W₄ are each independently selected from N and CH; said W₅ is selected from NH, CH₂, and 0;
or said L is selected from the following structures: 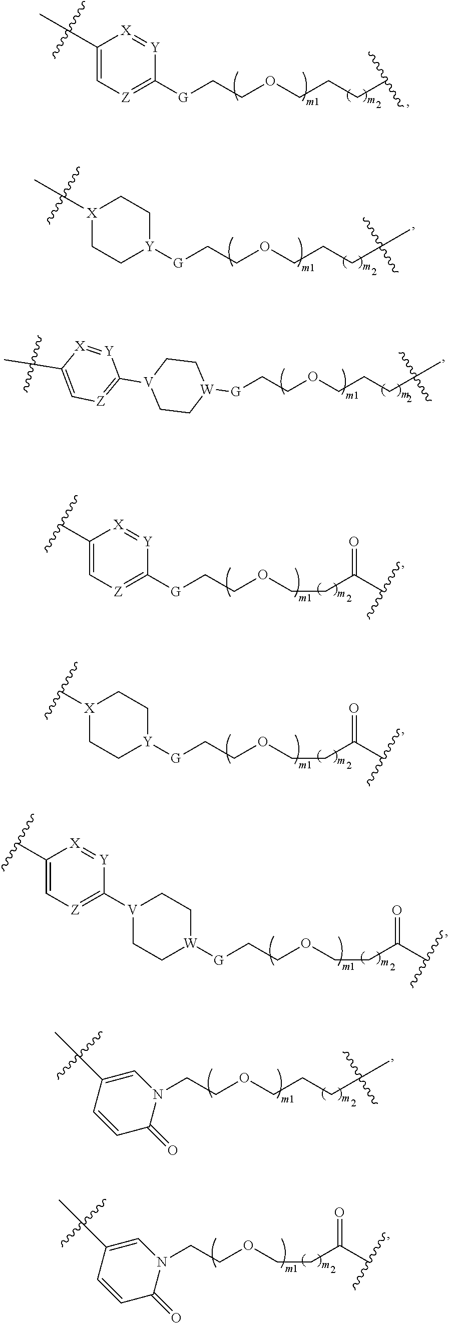
m1 is 0, 1, 2, 3, 4, 5, or 6, and m2 is 0, 1, 2, 3, 4, 5, or 6.

9. The small molecule conjugate according to claim 1, characterized in that: said L is selected from the following structures:

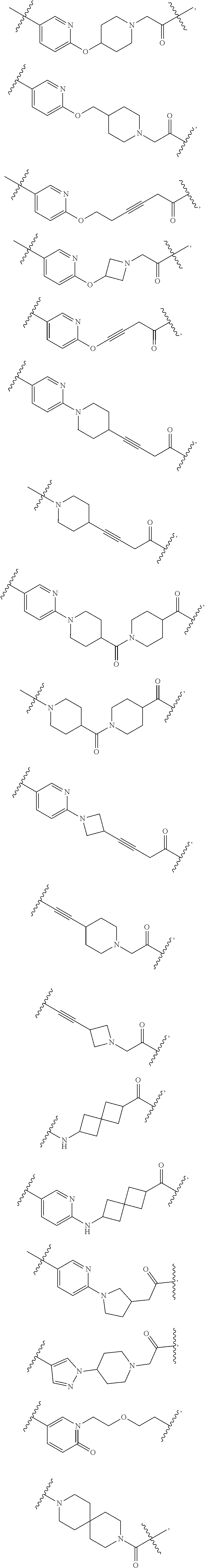

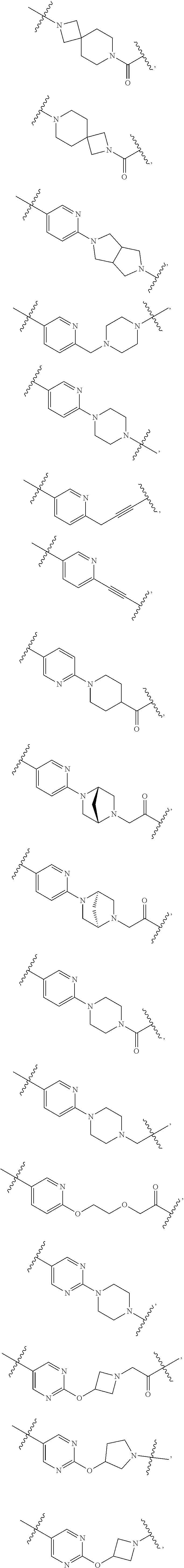

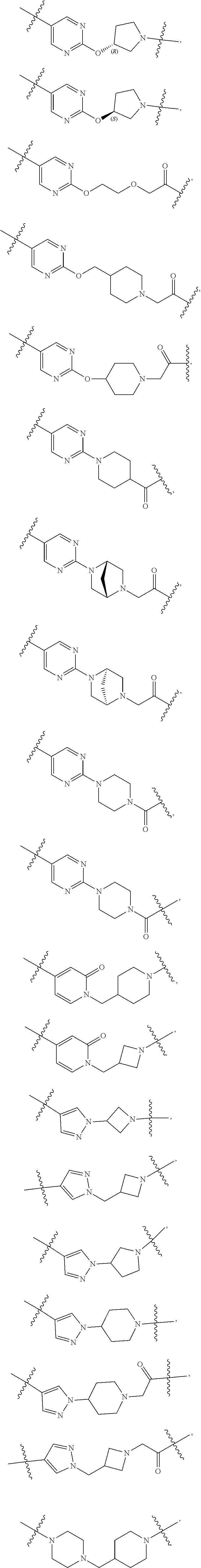

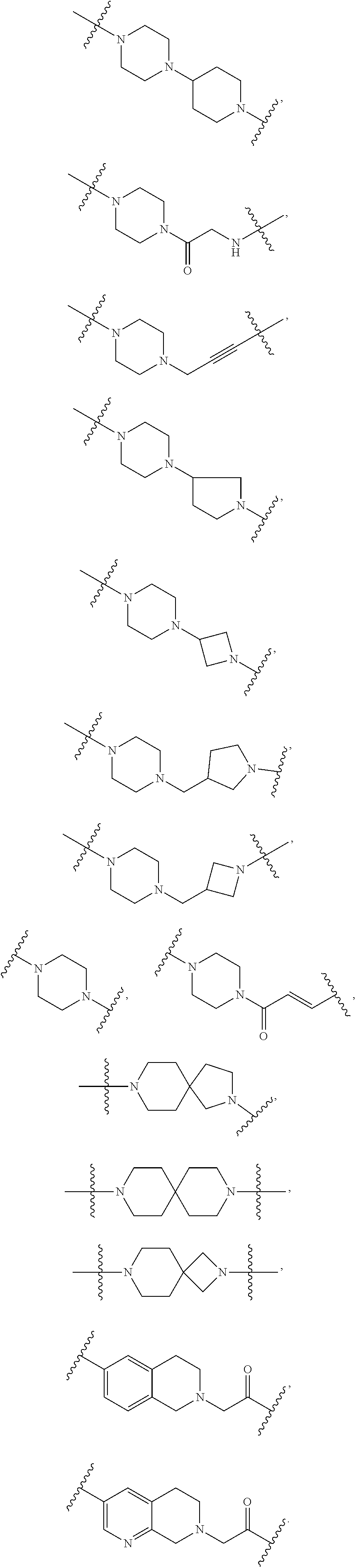

10. The small molecule conjugate according to claim 1, characterized in that:
The structural formula of the said small molecule conjugate is as shown in any one of formulae IV to XII: 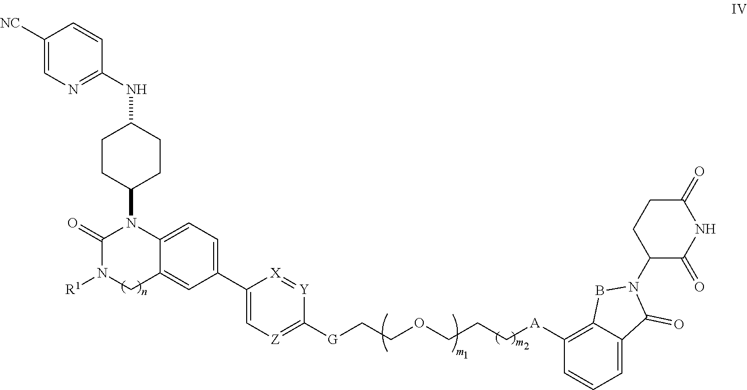
In Formula IV, m₁ is an integer from 0 to 6, m₂ is an integer from 0 to 15, A is CH₂, NH, O, or CO, G is absent, CH₂, NH, O, CONH, or NHCO, B is CO or CH₂, X, Y, and Z are each independently CH or N, and the definitions of R¹ and n are the same as in Formula II; 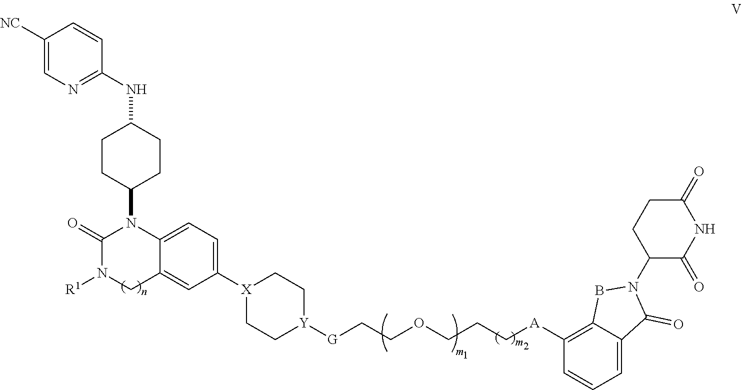
In Formula V, m₁ is an integer from 0 to 6, m₂ is an integer from 0 to 15, A is CH₂, NH, O, or CO, G is absent, CH₂, NH, O, CONH, or NHCO, B is CO or CH₂, X and Y are each independently CH or N, and the definitions of R¹ and n are the same as in Formula II; 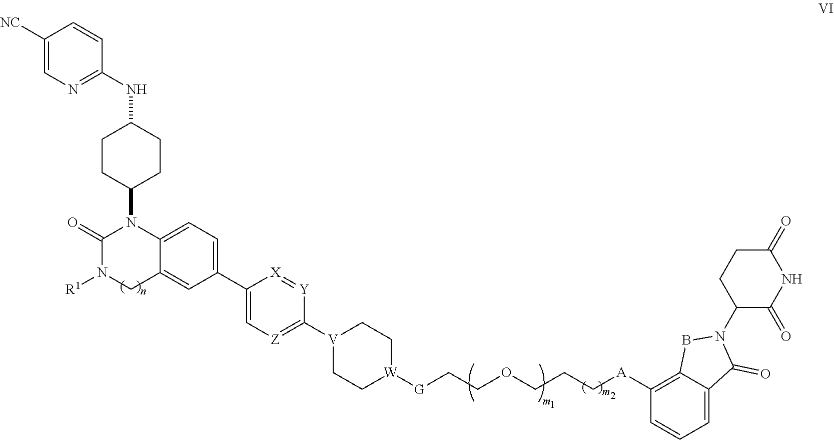
In Formula VI, m₁ is an integer from 0 to 6, m₂ is an integer from 0 to 15, A is CH₂, NH, O, or CO, G is absent, CH₂, NH, O, CONH, or NHCO, B is CO or CH₂, X, Y, and Z are each independently CH or N, V and W are each independently CH or N, and the definitions of R¹ and n are the same as in Formula II; 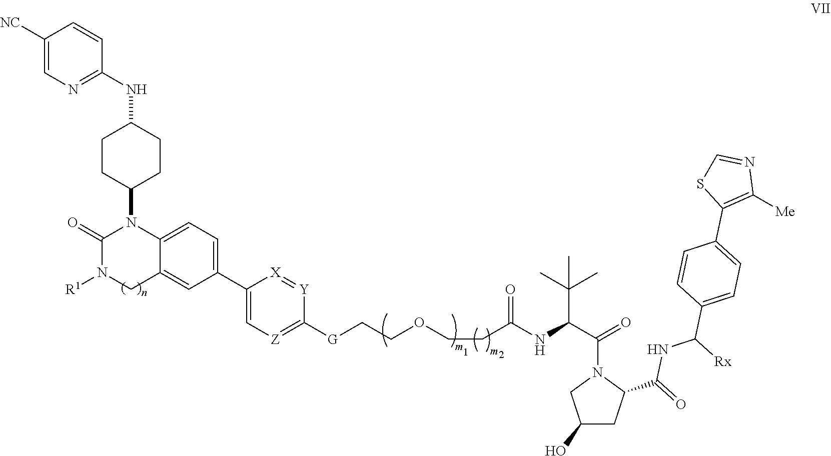
In Formula VII, m₁ is an integer from 0 to 6, m₂ is an integer from 0 to 15, G is absent, CH₂, NH, O, CONH, or NHCO; Rₓ is H or CH₃; X, Y, and Z are each independently CH or N; and the definitions of R¹ and n are the same as in Formula II; 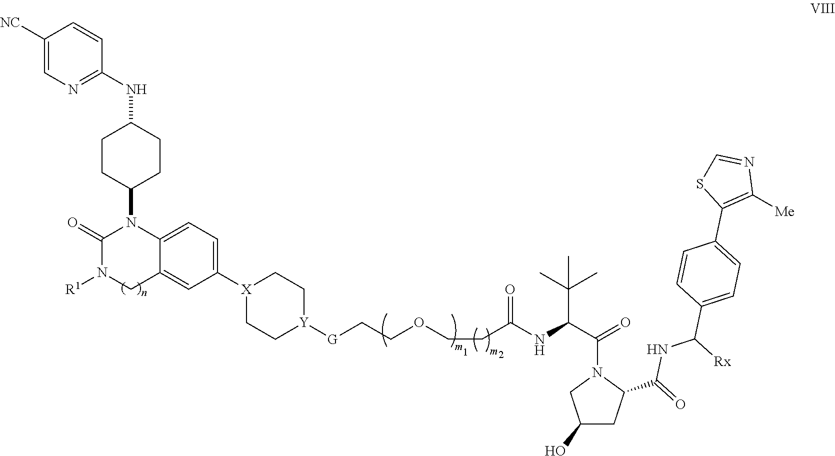
In Formula VIII, m₁ is an integer from 0 to 6, m₂ is an integer from 0 to 15, G is absent, CH₂, NH, O, CONH, or NHCO, Rₓ is H or CH₃, X and Y are each independently CH or N, and the definitions of R¹ and n are the same as in Formula II; 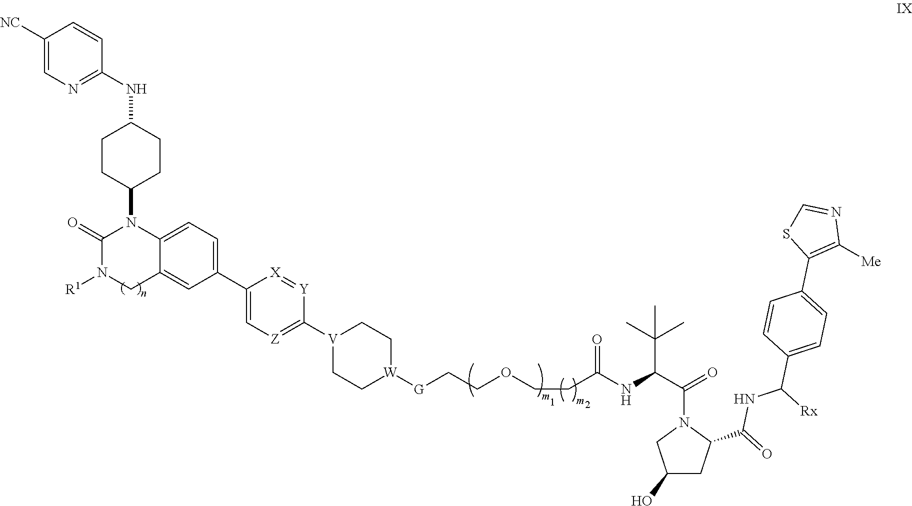
In Formula IX, m₁ is an integer from 0 to 6, m₂ is an integer from 0 to 15, G is absent, CH₂, NH, O, CONH, or NHCO; Rₓ is H or CH₃; X, Y, and Z are each independently CH or N, V and W are each independently CH or N, and the definitions of R¹ and n are the same as in Formula II; 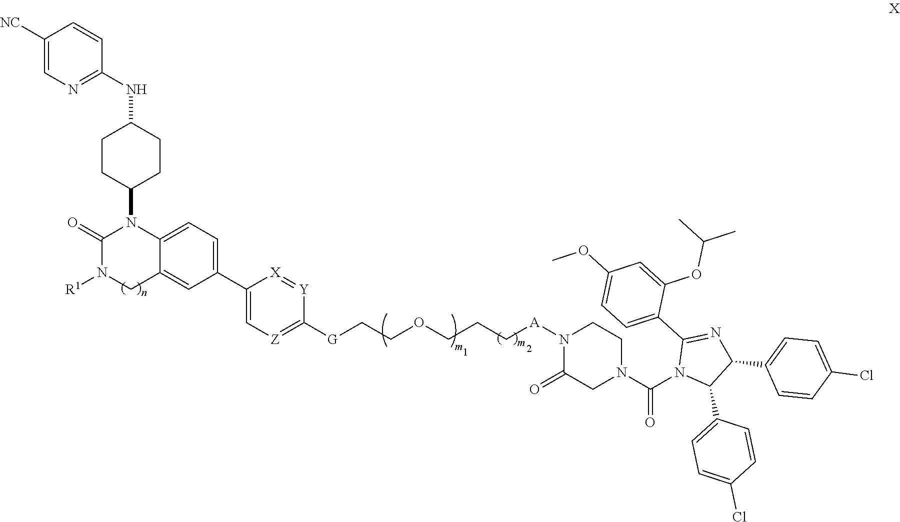
In Formula X, m₁ is an integer from 0 to 6, m₂ is an integer from 0 to 15, G is absent, CH₂, NH, O, CONH, or NHCO; A is CH₂ or CO; X, Y, and Z are each independently CH or N, and the definitions of R¹ and n are the same as in Formula II; 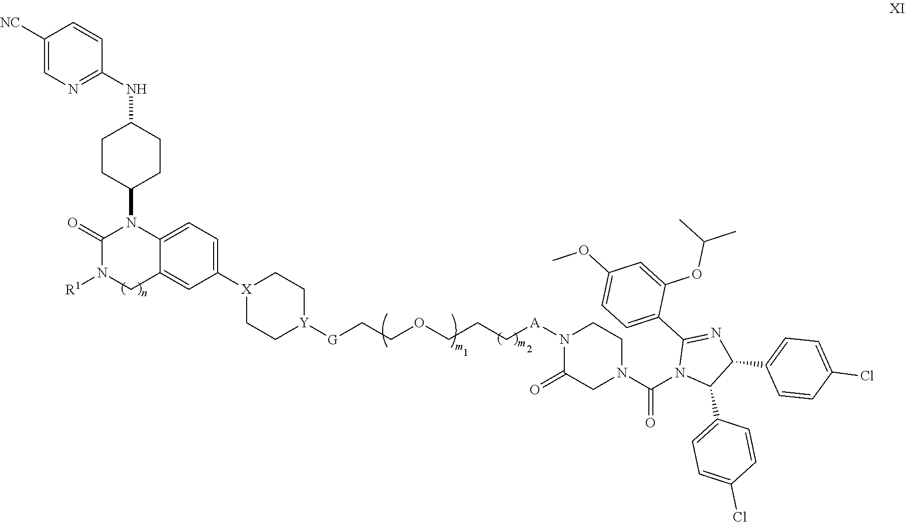
In Formula XI, m₁ is an integer from 0 to 6, m₂ is an integer from 0 to 15, G is absent, CH₂, NH, O, CONH, or NHCO; A is CH₂ or CO; X and Y are each independently CH or N, and the definitions of R¹ and n are the same as in Formula II; 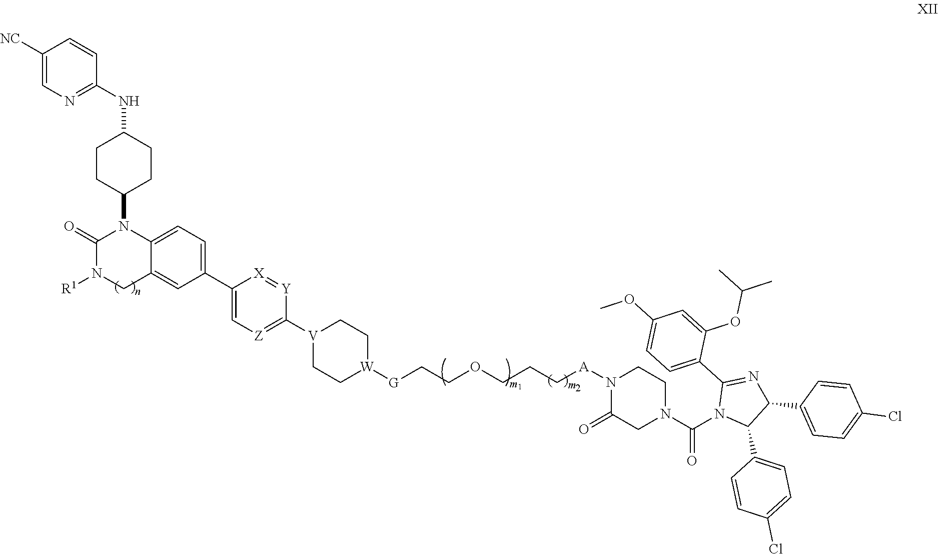
In Formula XII, m₁ is an integer from 0 to 6, m₂ is an integer from 0 to 15, G is absent, CH₂, NH, O, CONH, or NHCO; A is CH₂ or CO; X, Y, and Z are each independently CH or N; V and W are each independently CH or N; and the definitions of R¹ and n are the same as in Formula II.

11. The small molecule conjugate according to claim 9, characterized in that: the said small molecule conjugate has a structure as shown in Formula IX, wherein n=0; R¹ is benzyl; X is CH; at least one of Y and Z is N, and when they are not both N, the other is CH; G is absent; m₁ is 0; m₂ is 0, 1, or 2; and Rₓ is methyl.

12. The small molecule conjugate according to claim 1, characterized in that: the said small molecule conjugate is a compound as shown in any one of the following formulae 1 to 70: 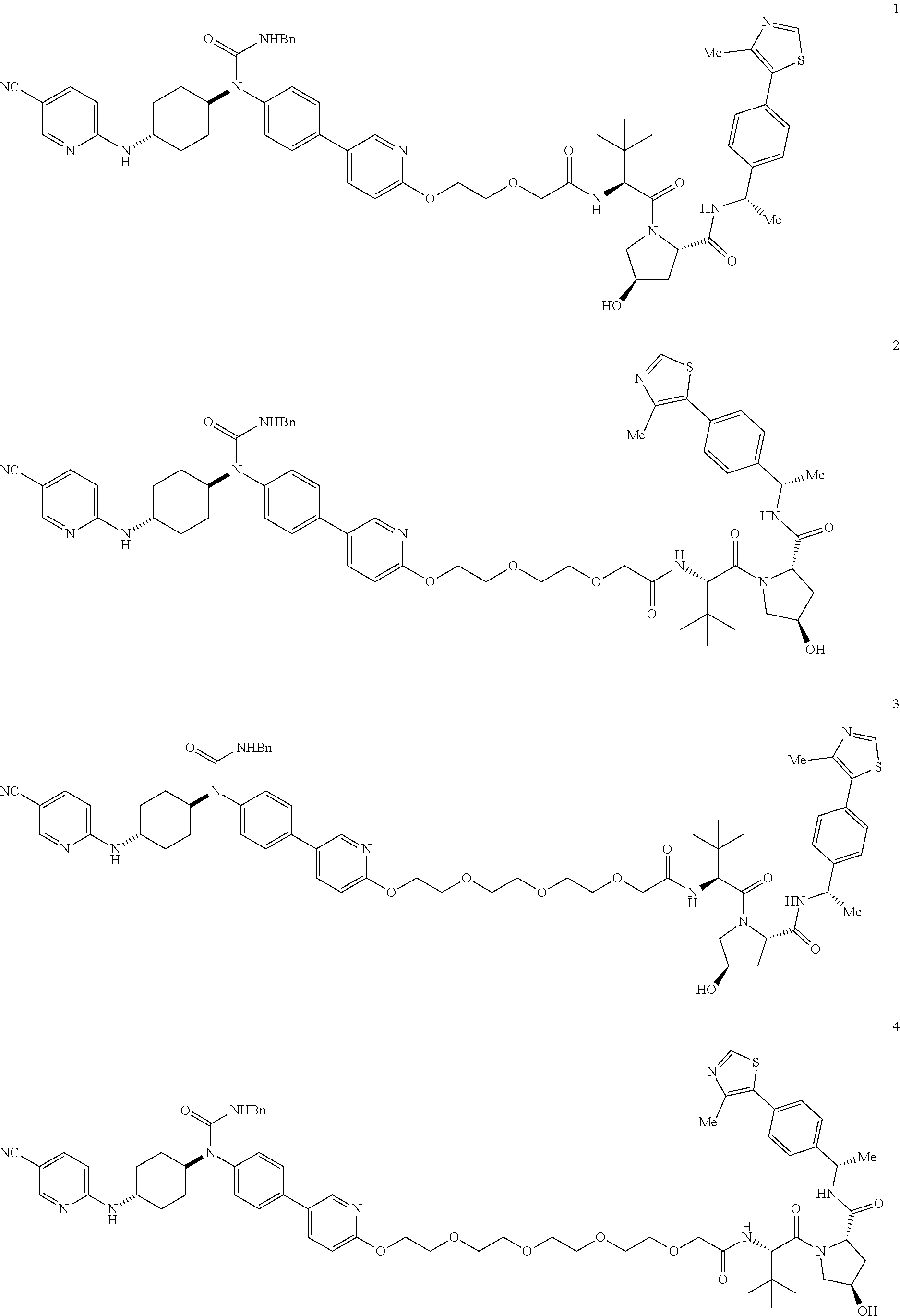 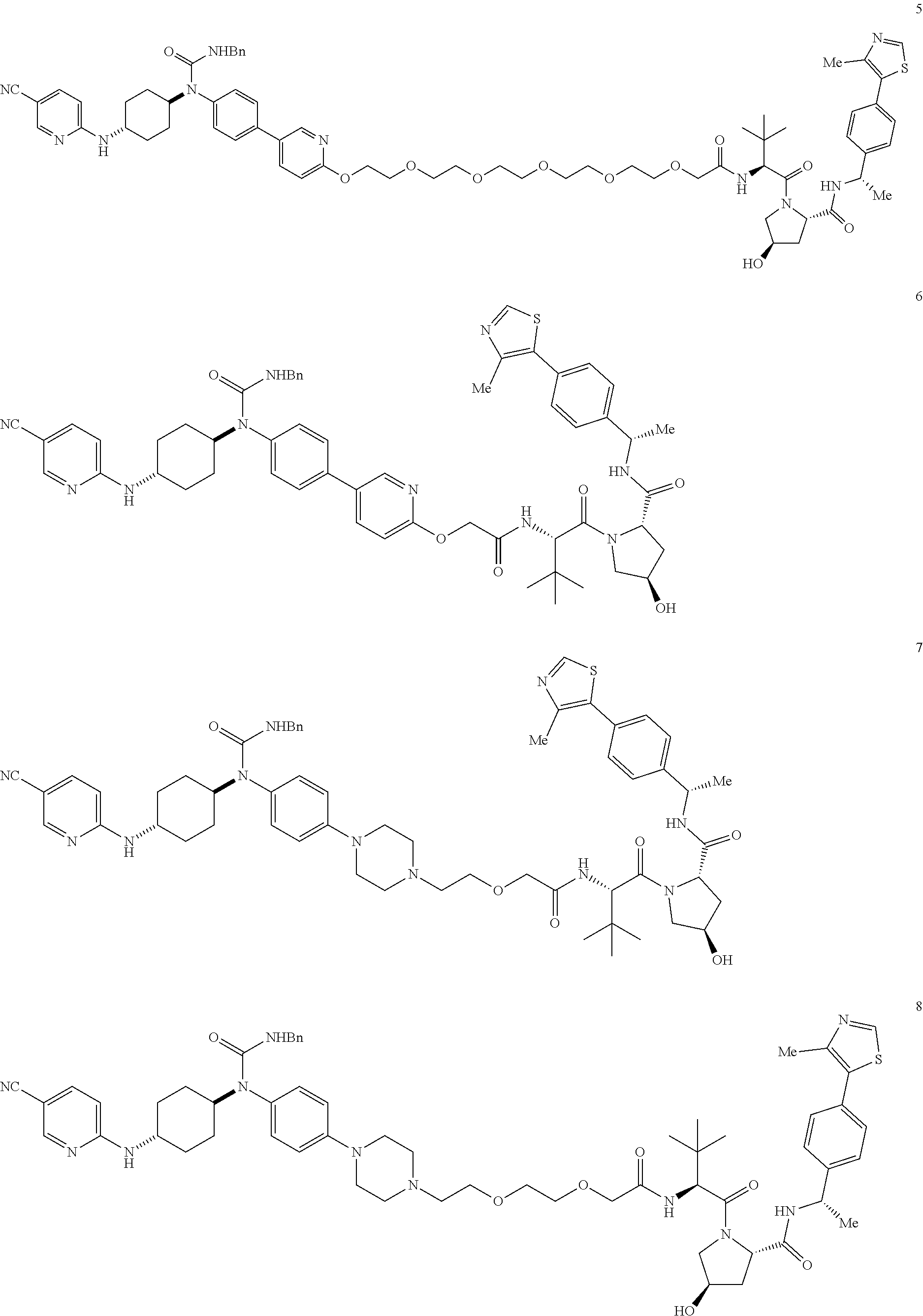 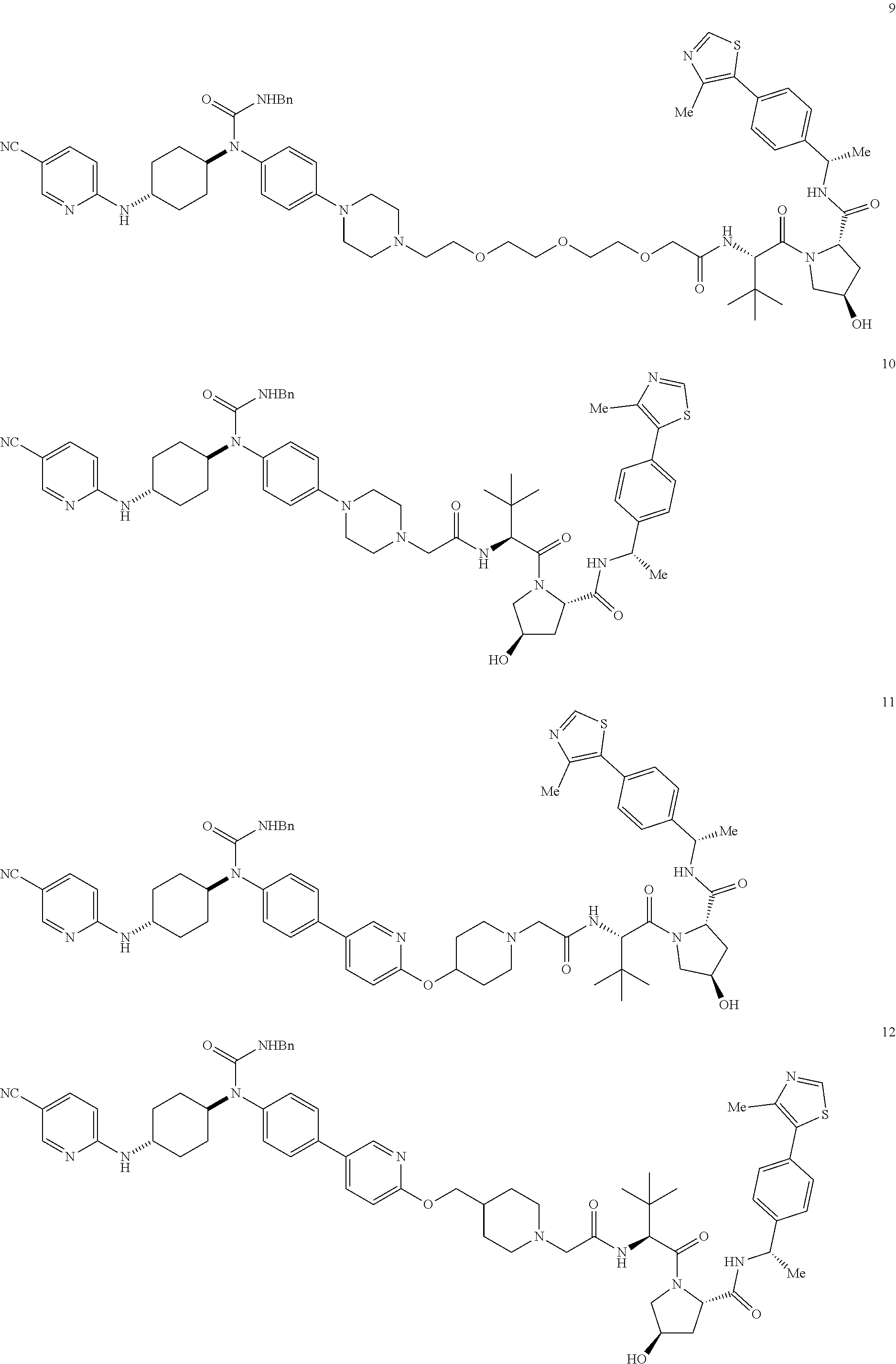 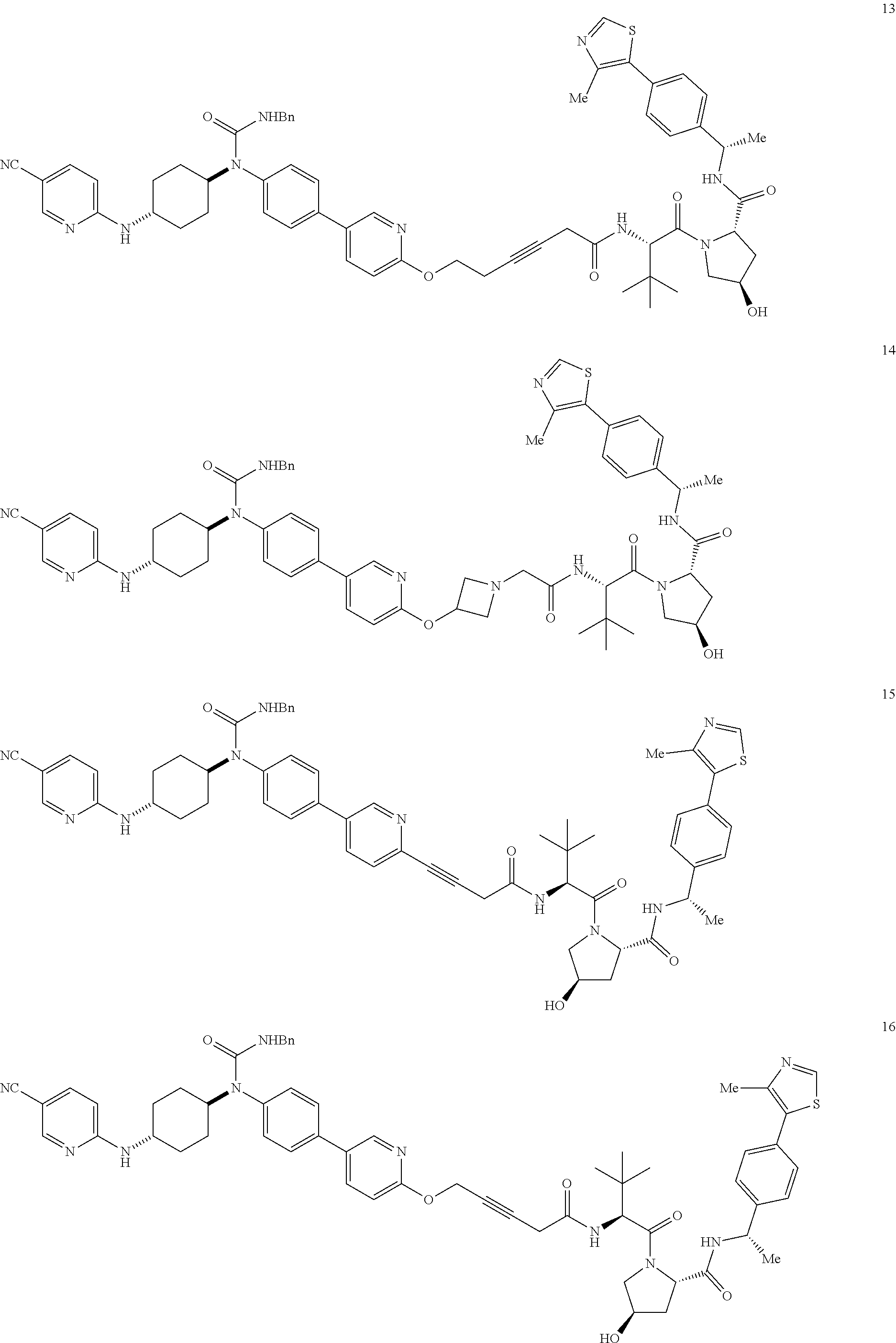 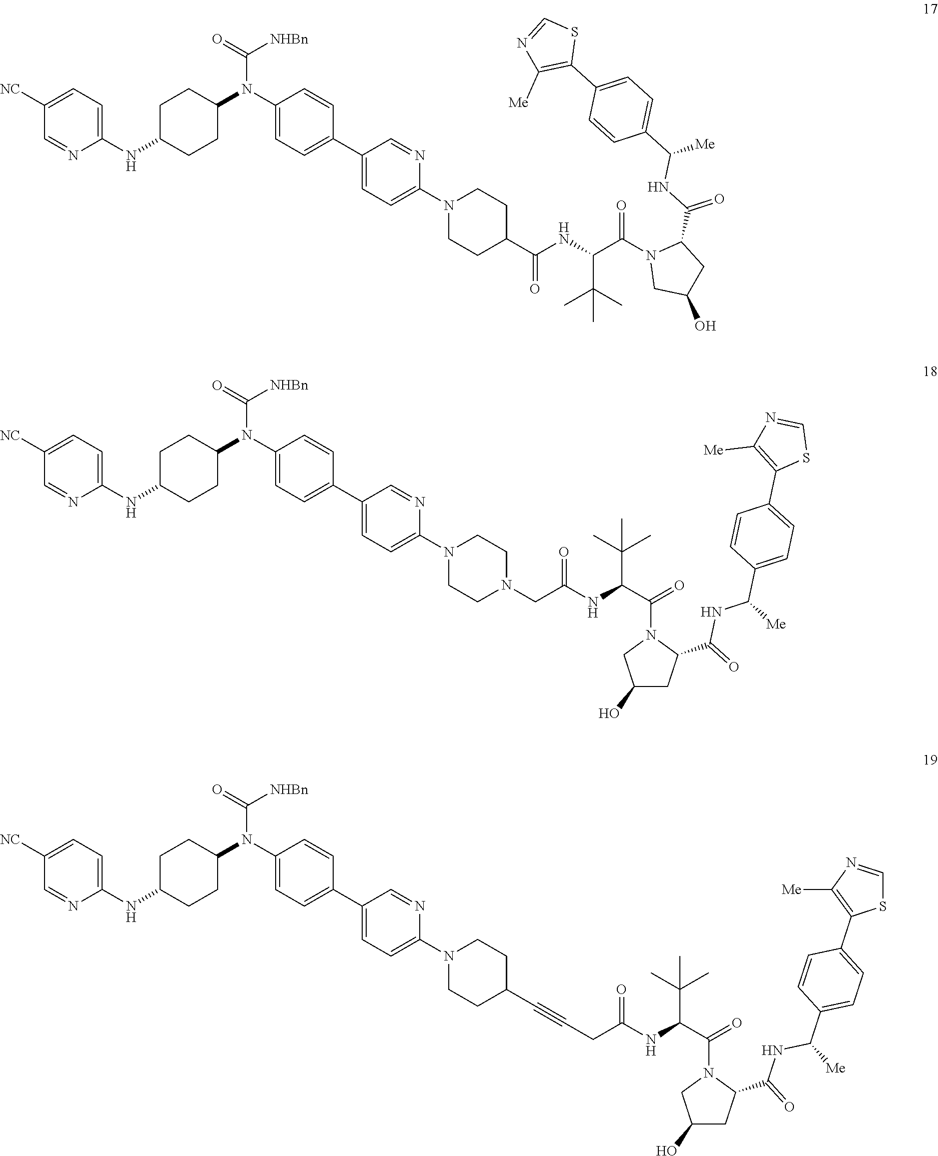 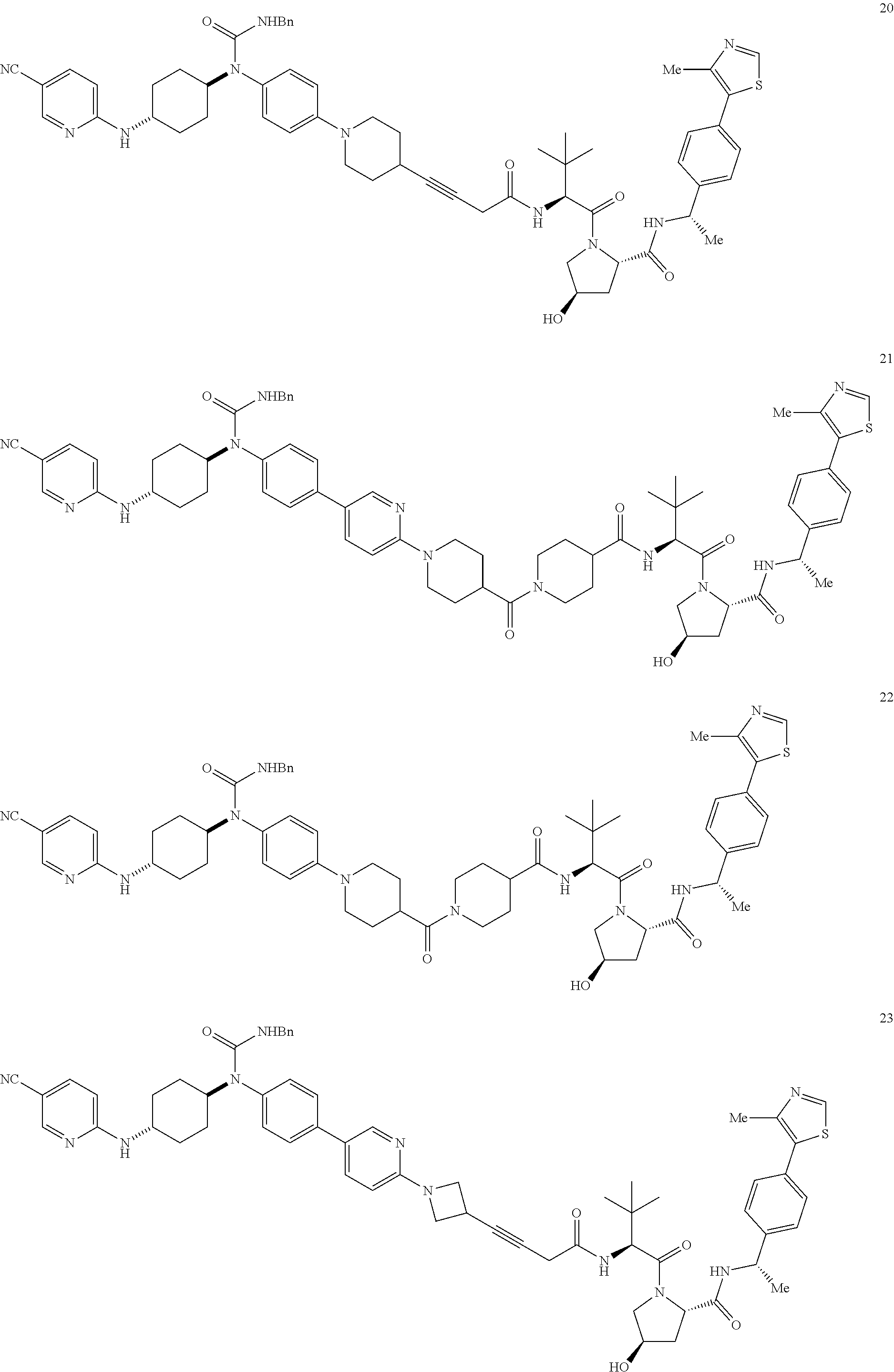 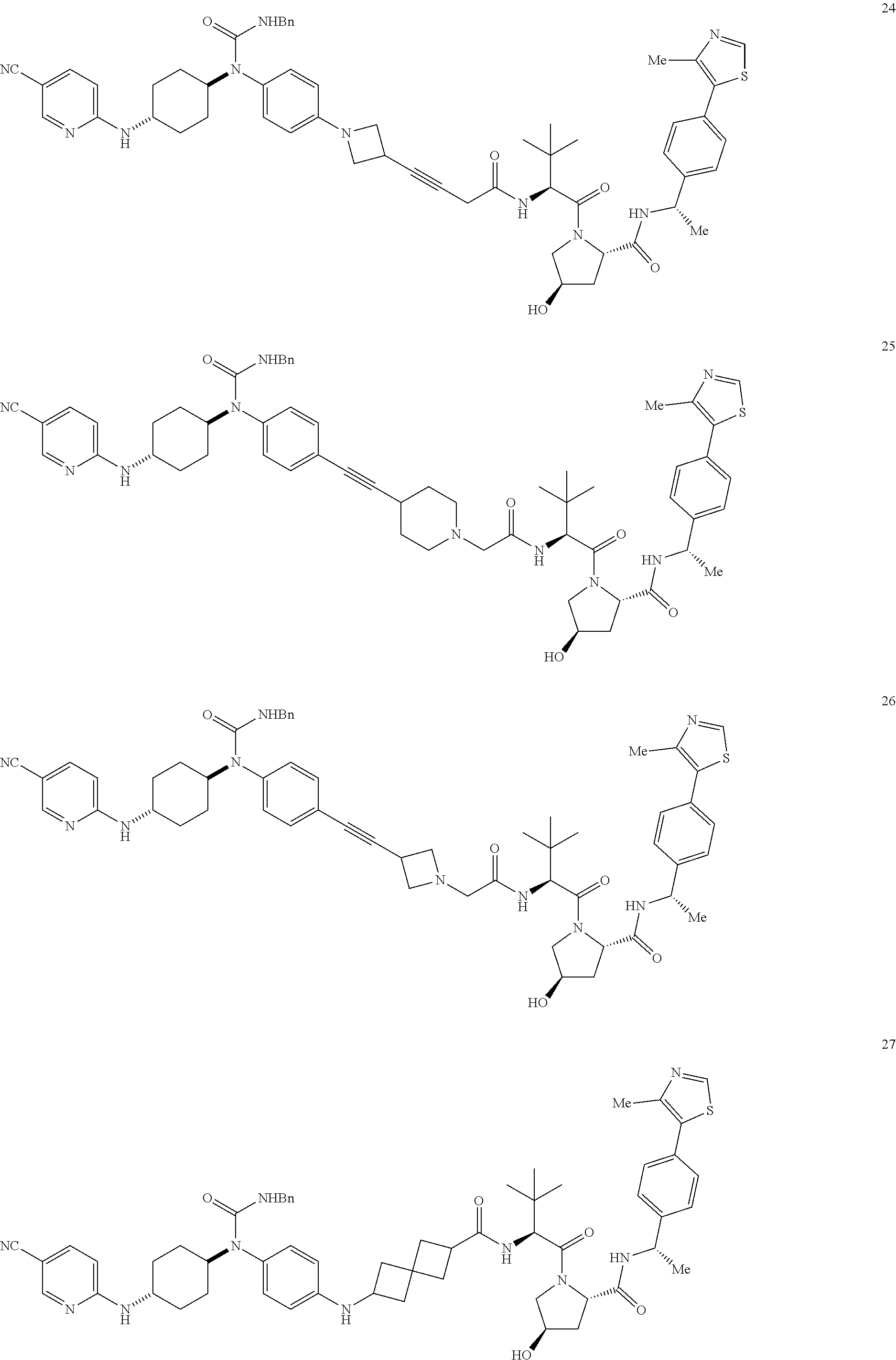 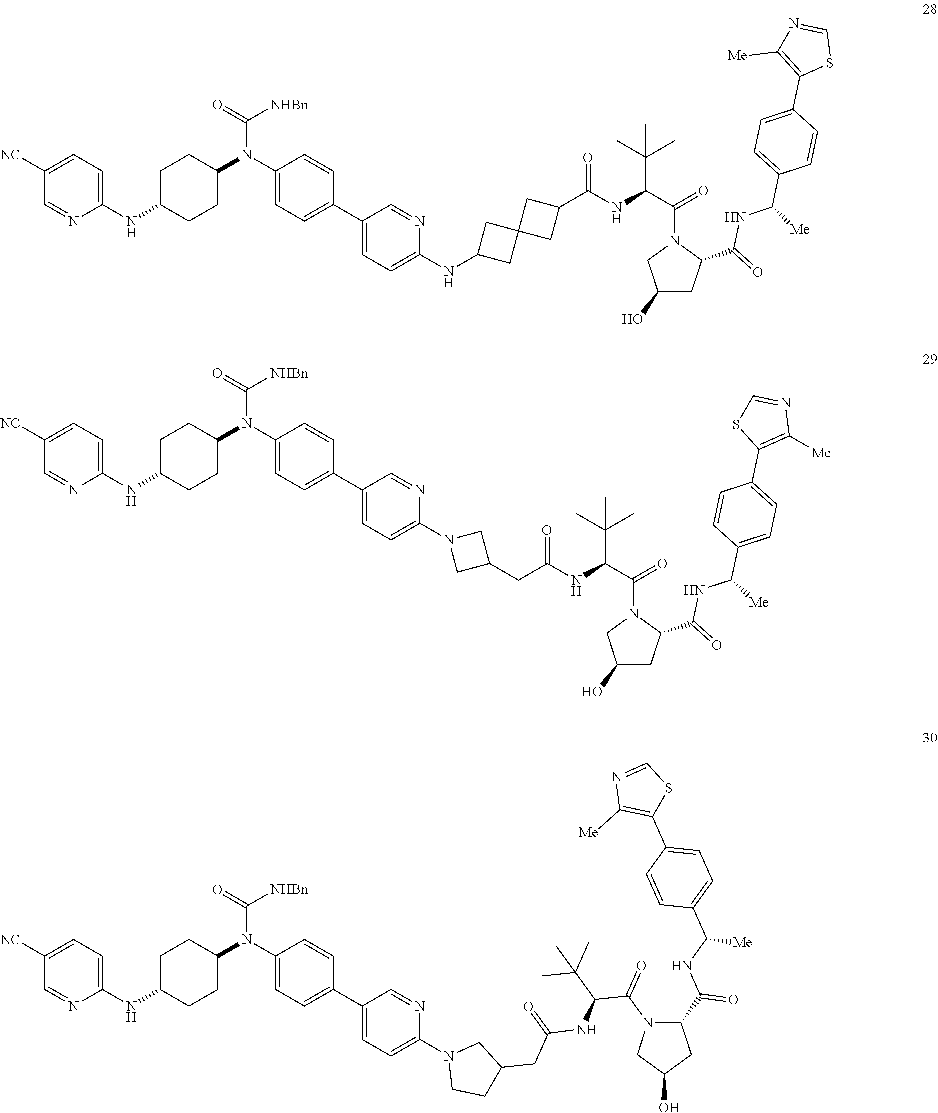 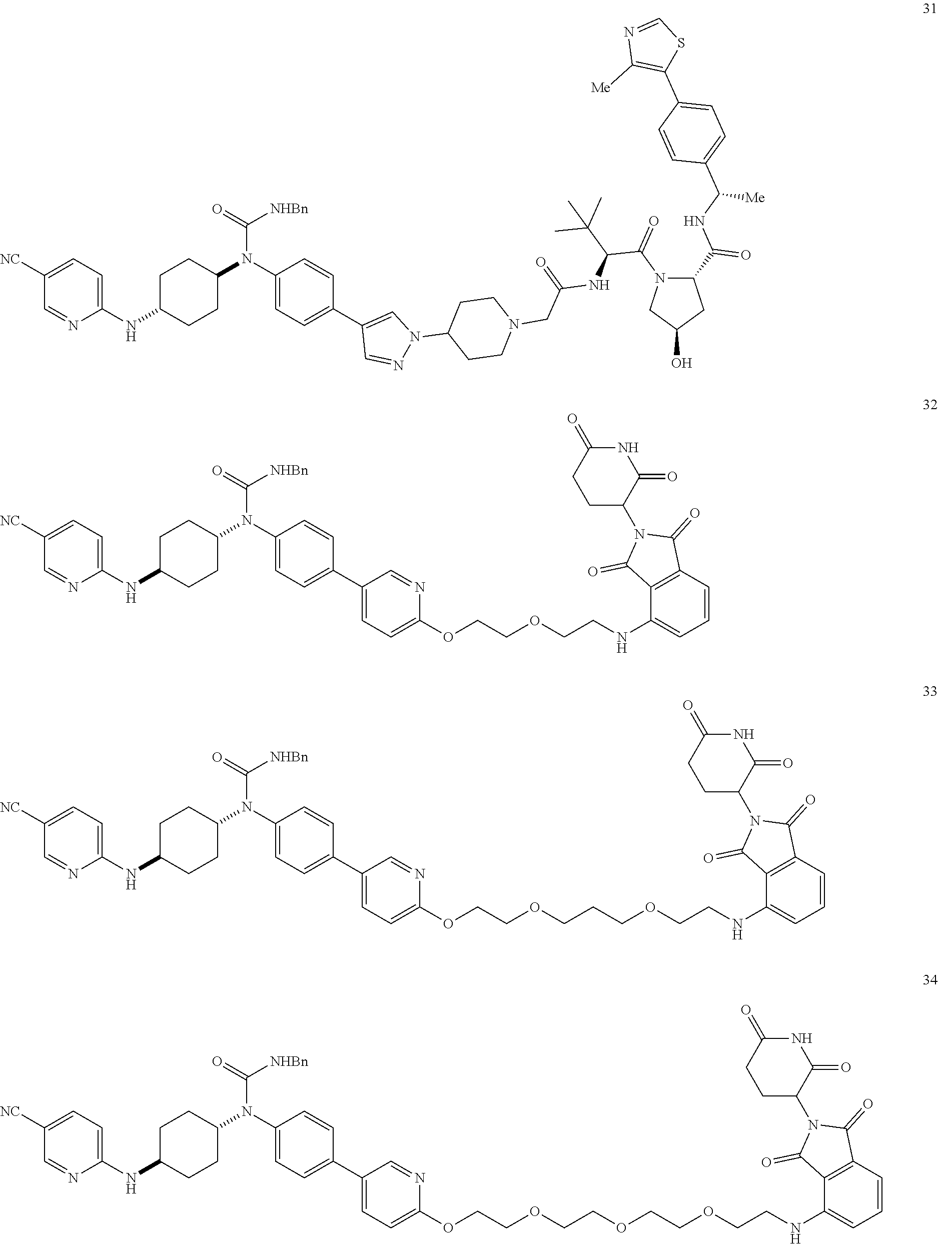 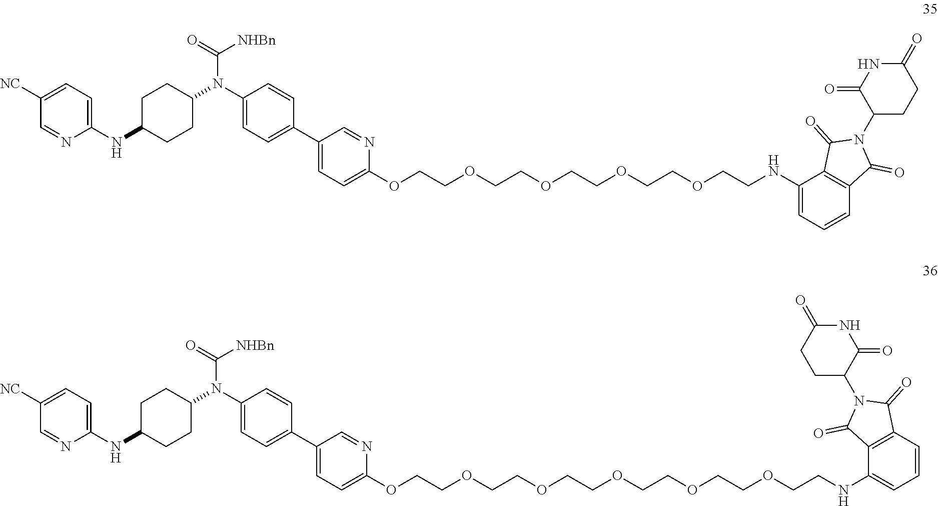 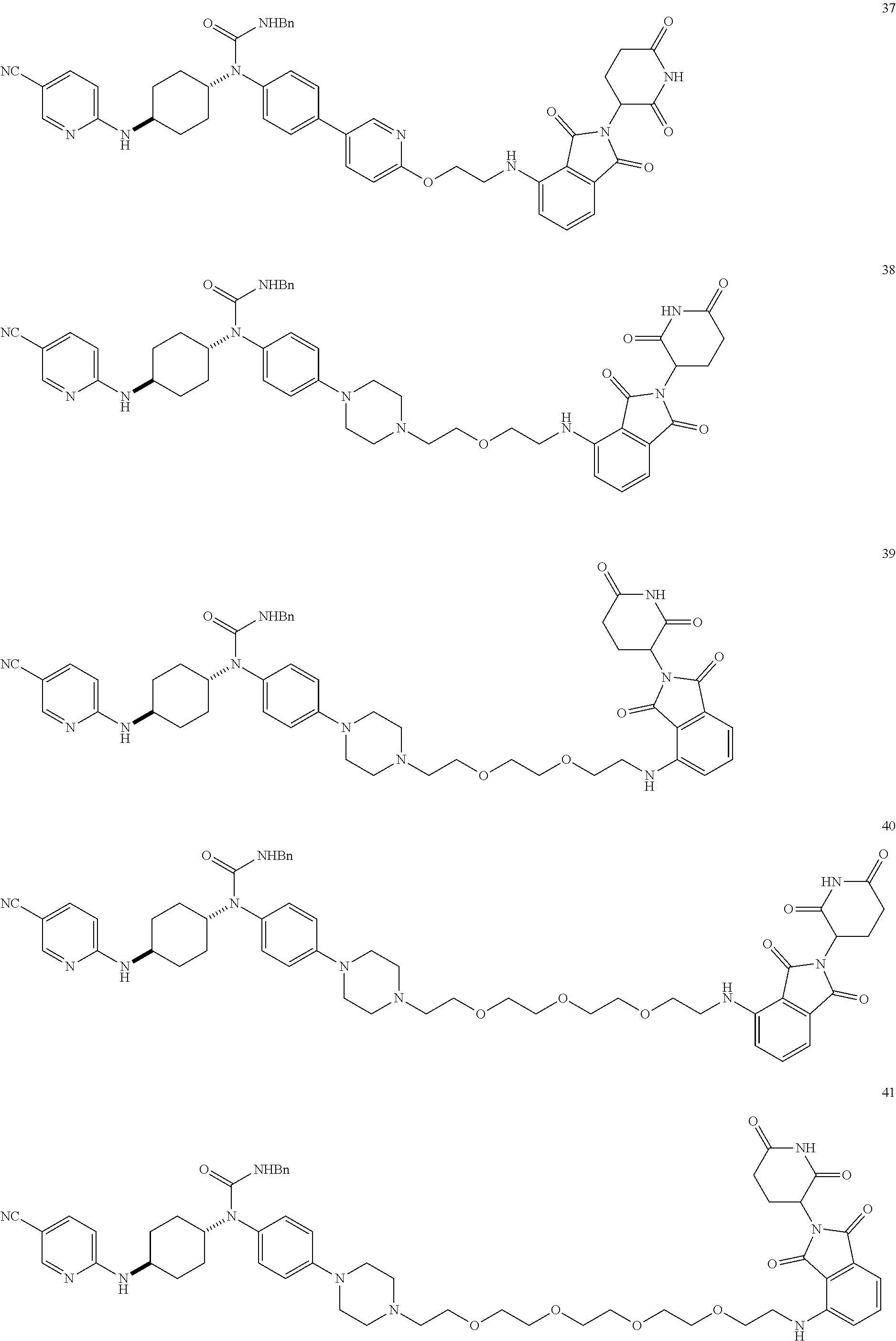 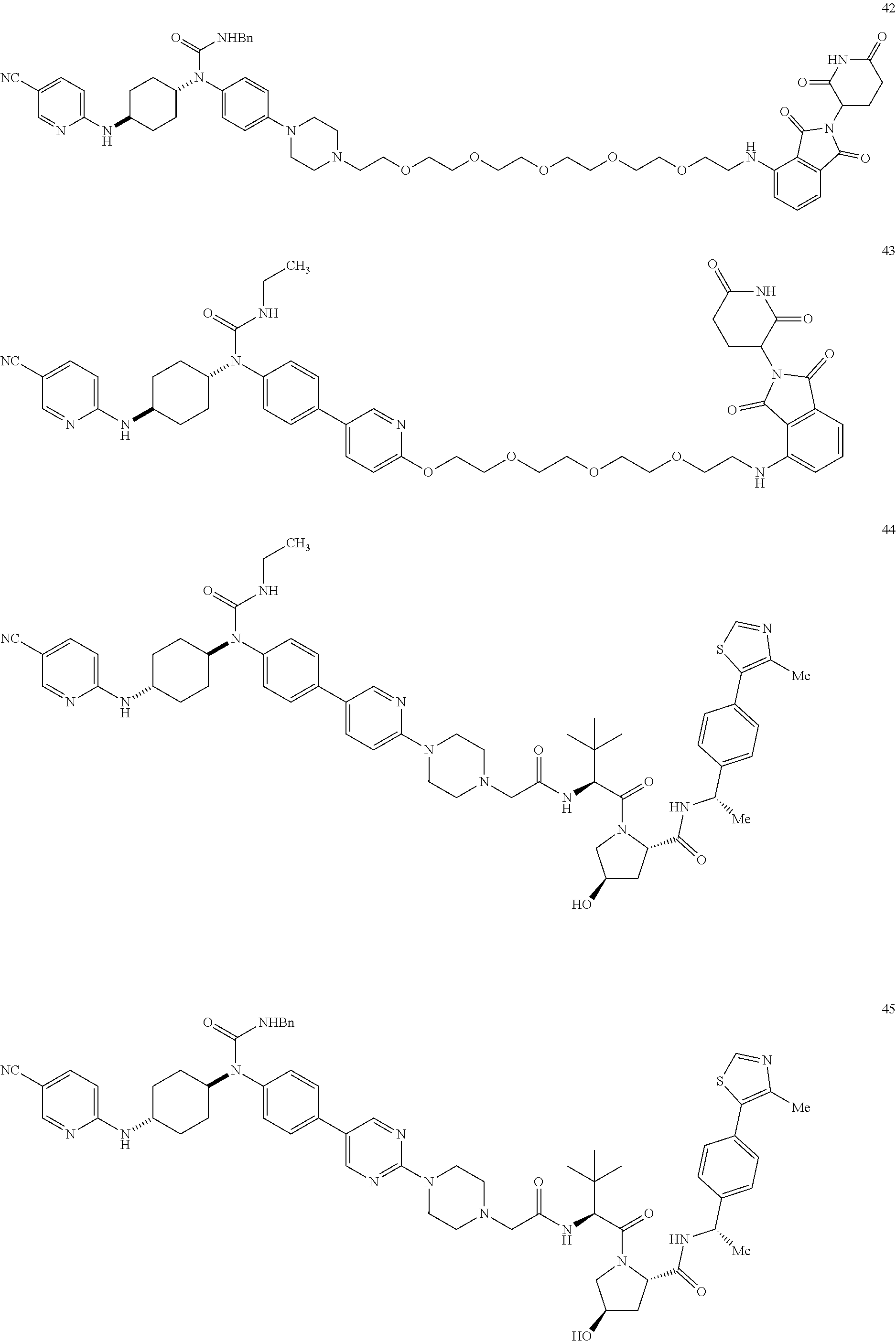 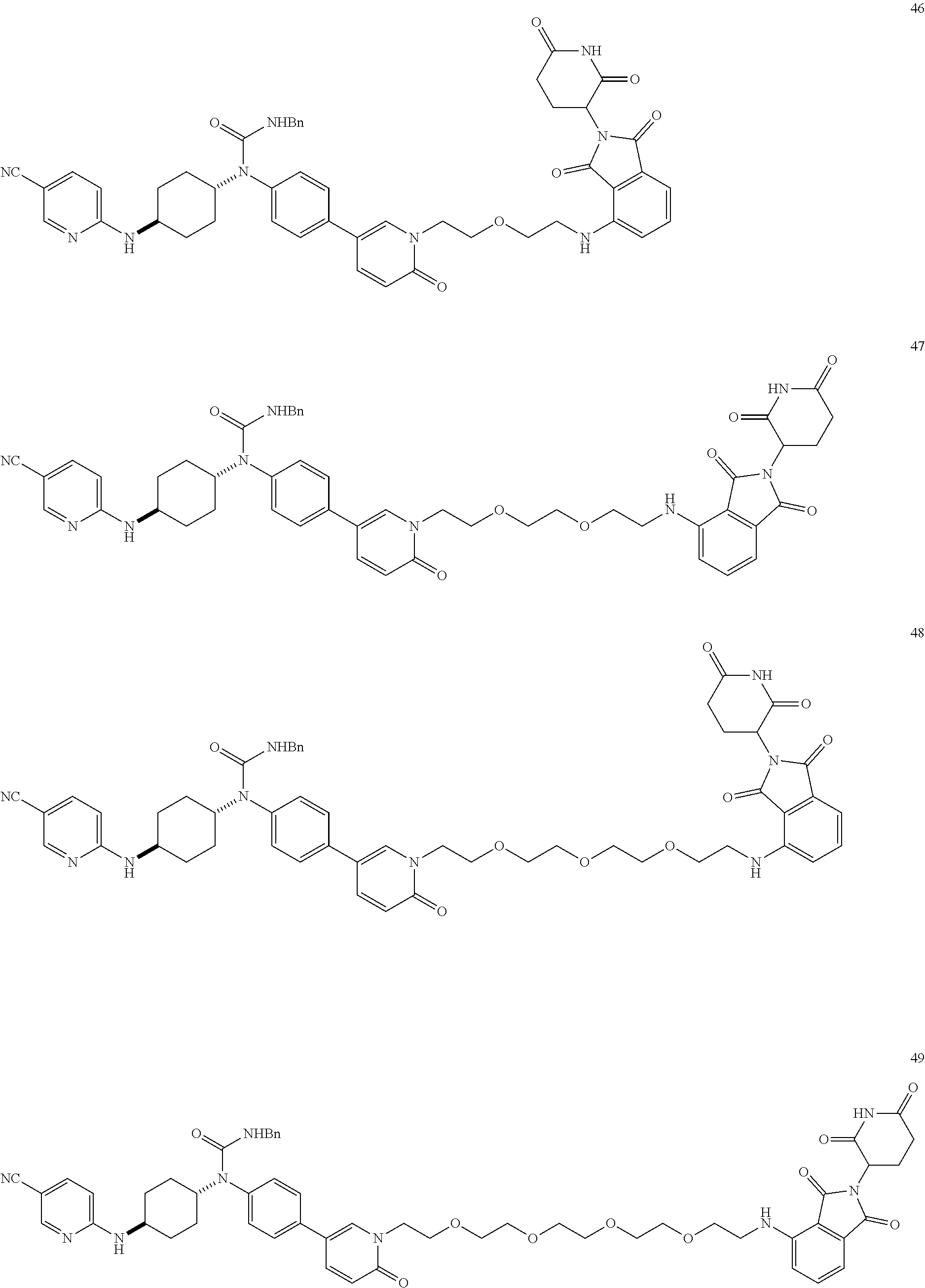 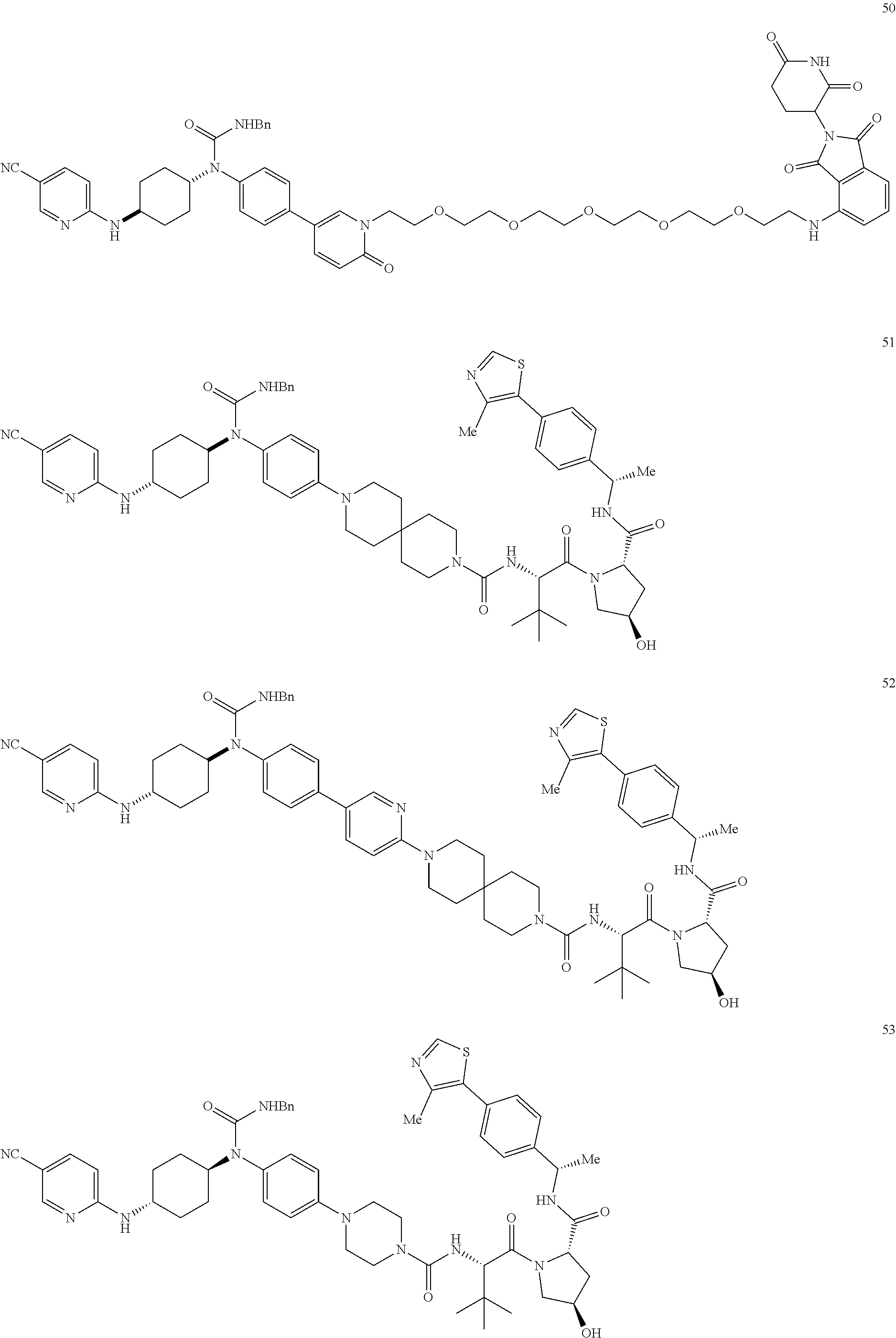 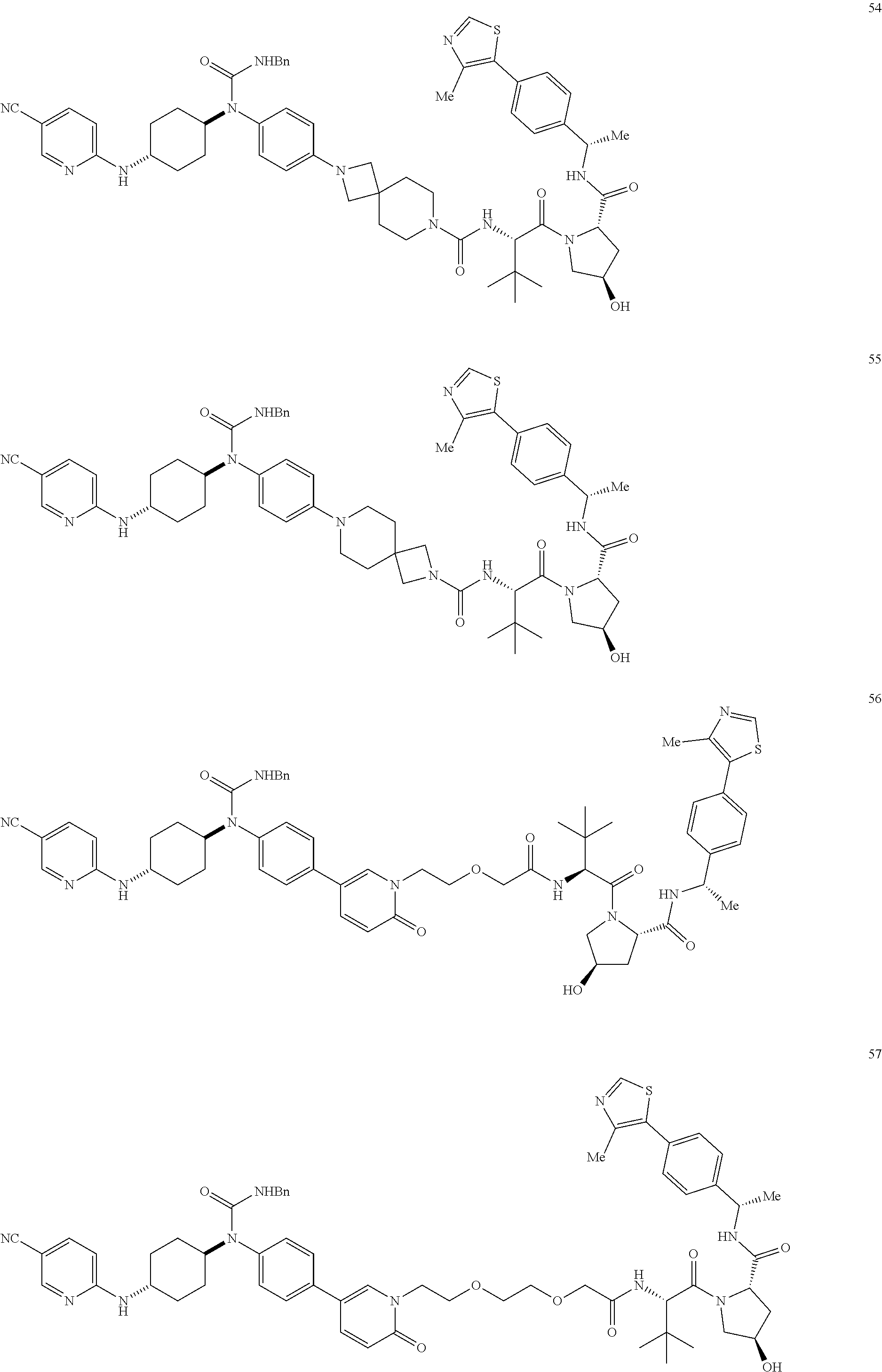 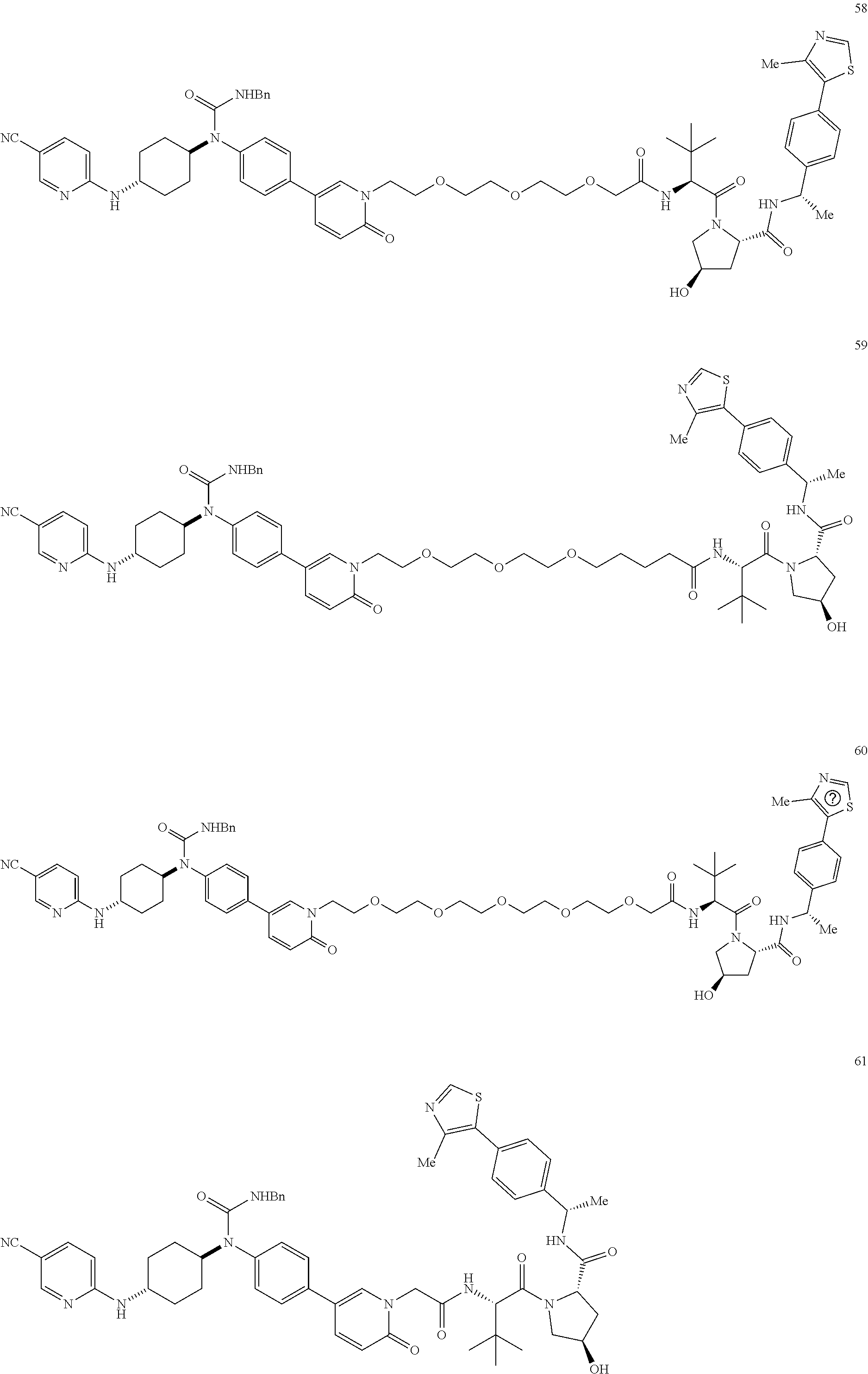 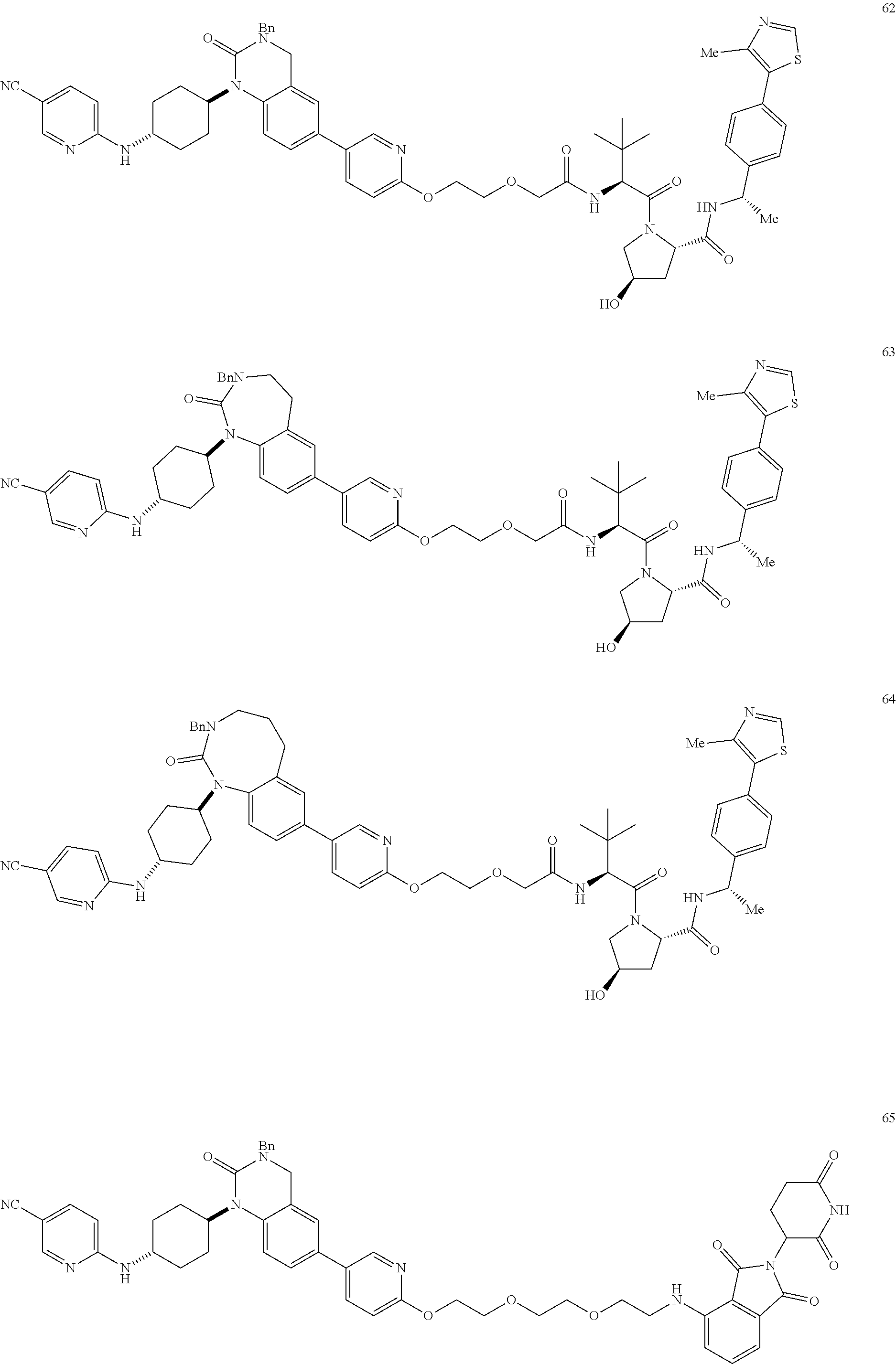 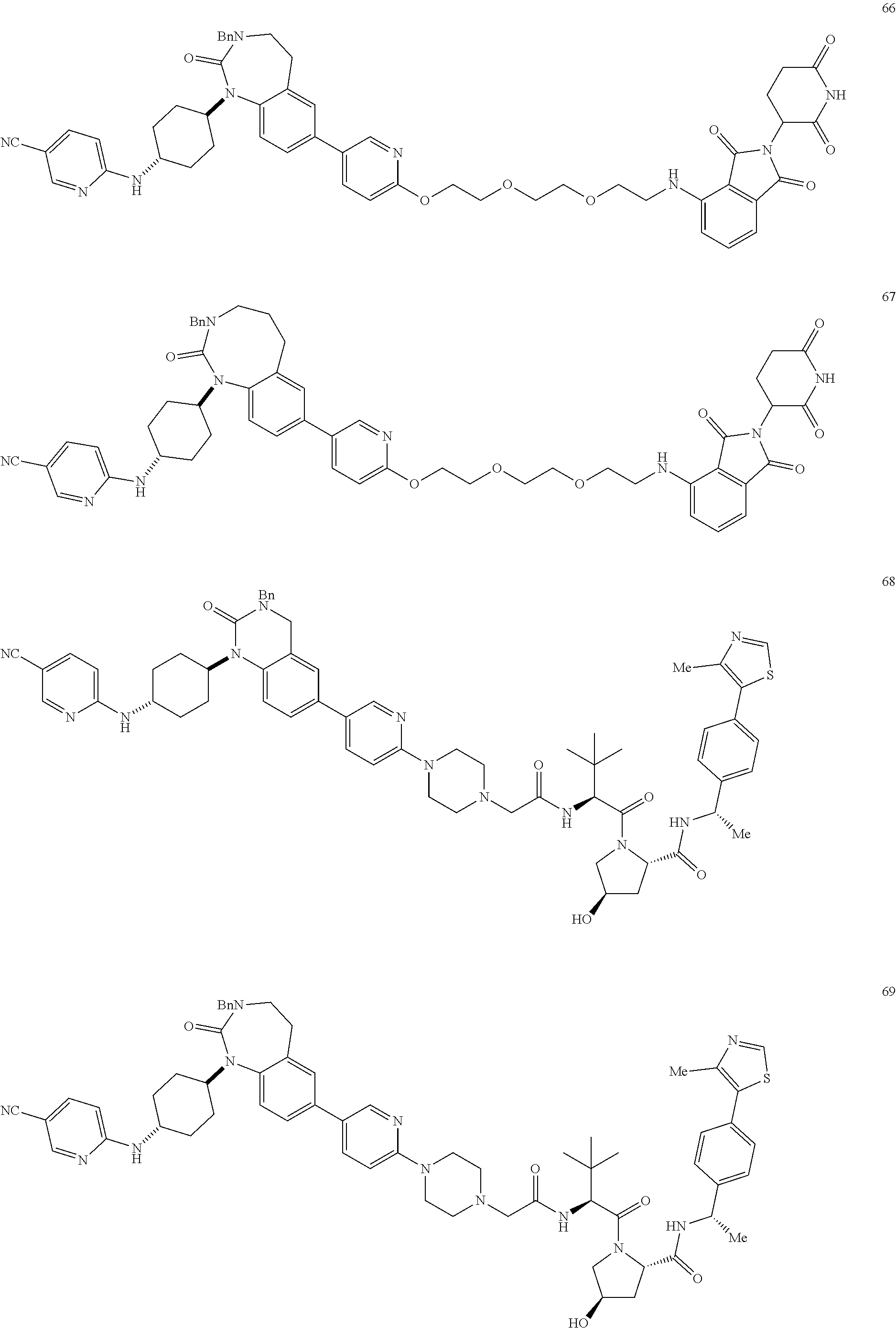 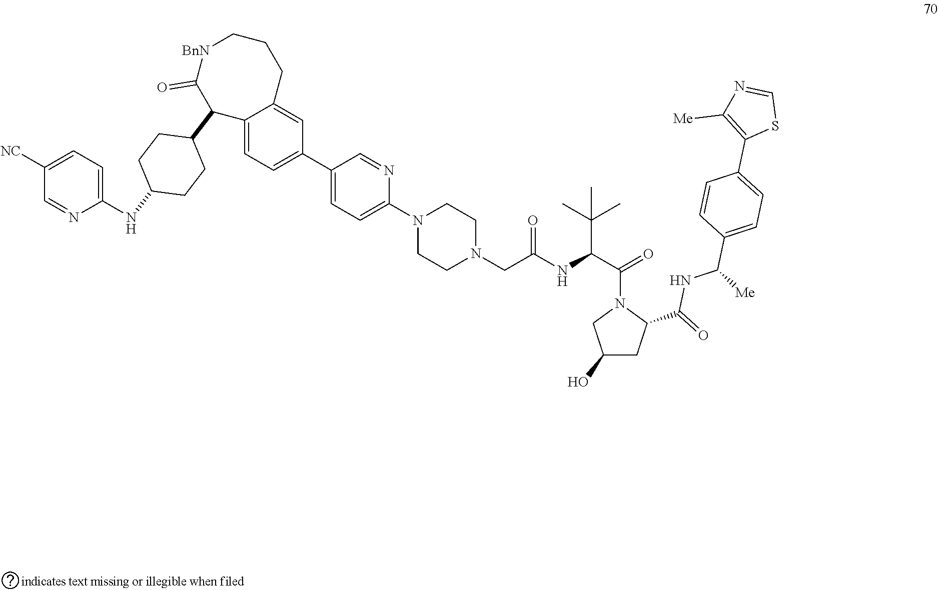
or selected from:

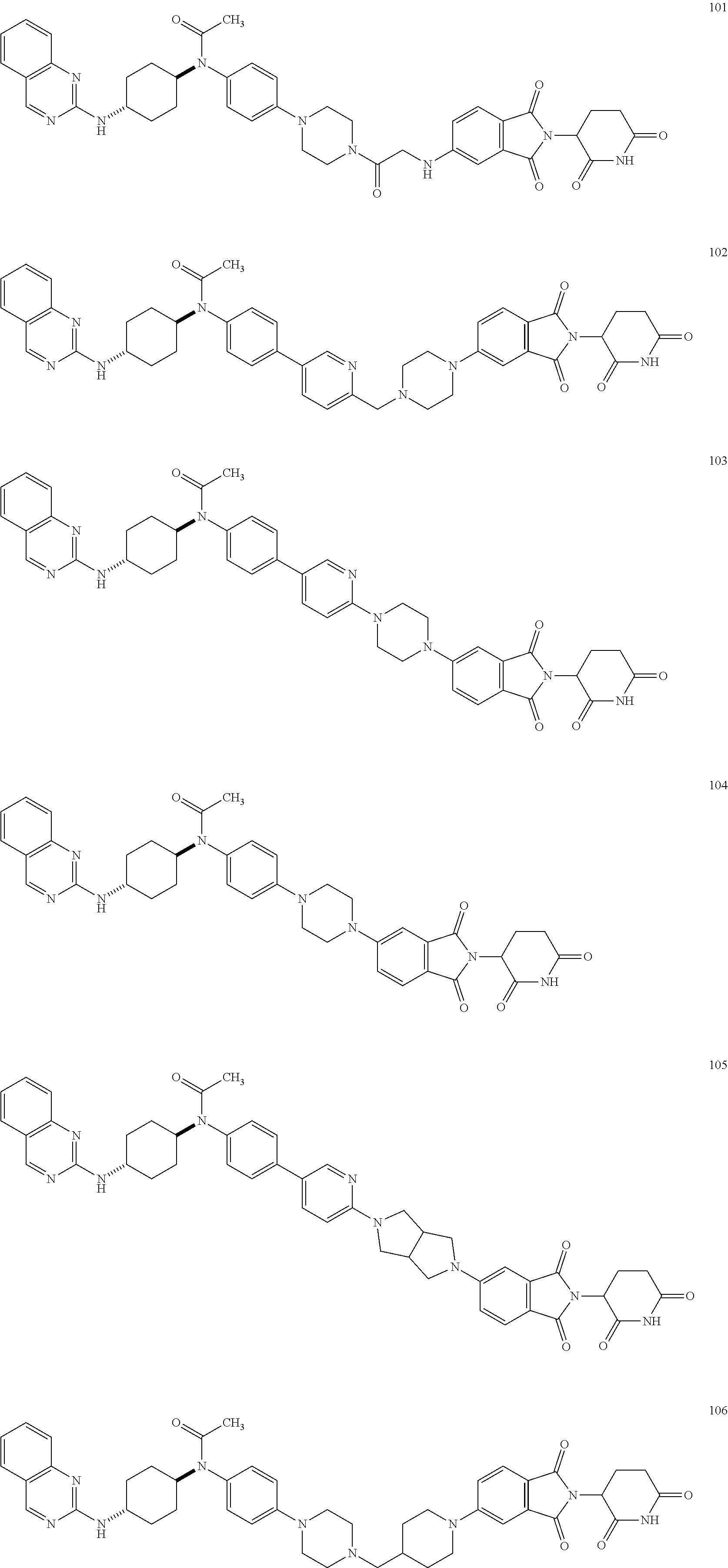

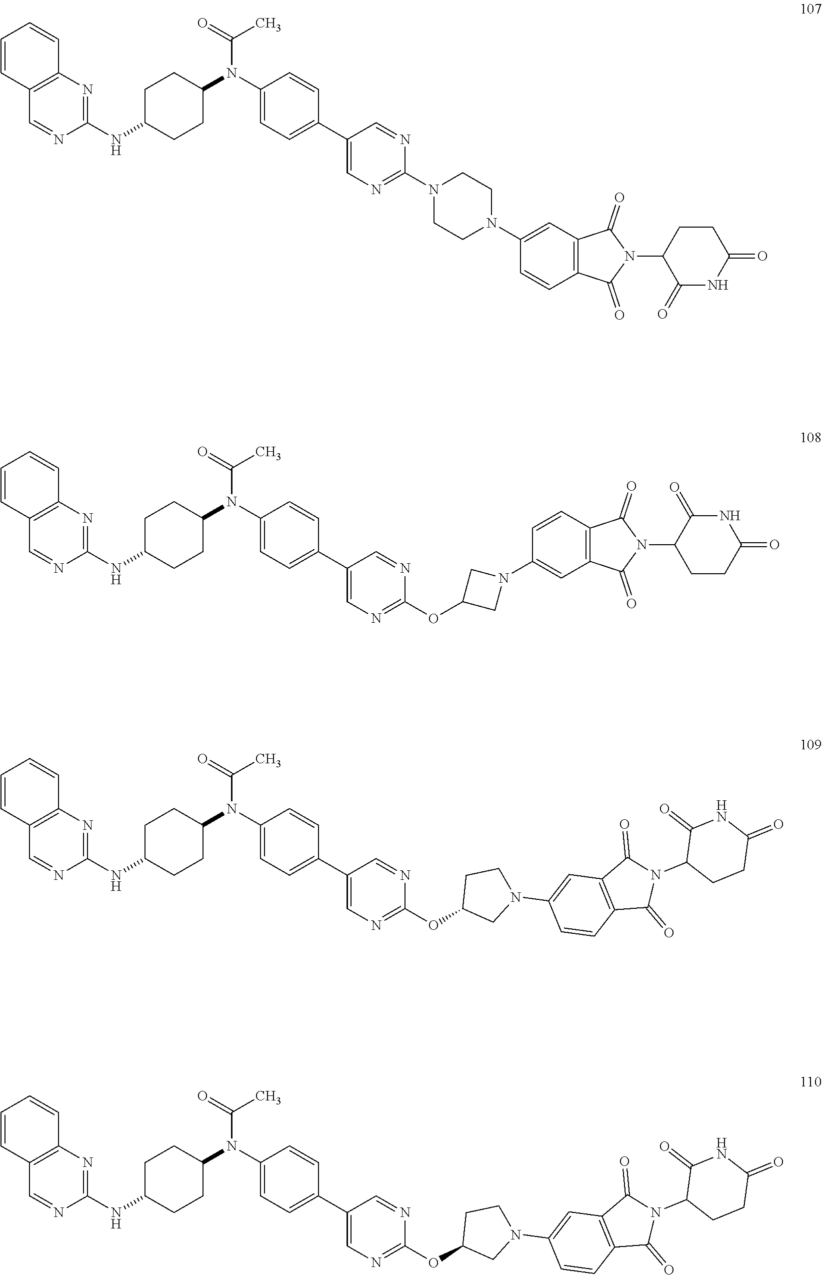 [SMILES: CC(=O)N(c1ccc(-c2cnc(N3CCN(c4ccc5c(c4)C(=O)N(C4CCC(=O)NC4=O)C5=O)CC3)nc2)cc1)[C@H]1CC[C@H](Nc2ncc3ccccc3n2)CC1 ; CC(=O)N(c1ccc(-c2cnc(OC3CN(c4ccc5c(c4)C(=O)N(C4CCC(=O)NC4=O)C5=O)C3)nc2)cc1)[C@H]1CC[C@H](Nc2ncc3ccccc3n2)CC1 ; CC(=O)N(c1ccc(-c2cnc(O[C@@H]3CCN(c4ccc5c(c4)C(=O)N(C4CCC(=O)NC4=O)C5=O)C3)nc2)cc1)[C@H]1CC[C@H](Nc2ncc3ccccc3n2)CC1 ; CC(=O)N(c1ccc(-c2cnc(O[C@H]3CCN(c4ccc5c(c4)C(=O)N(C4CCC(=O)NC4=O)C5=O)C3)nc2)cc1)[C@H]1CC[C@H](Nc2ncc3ccccc3n2)CC1]

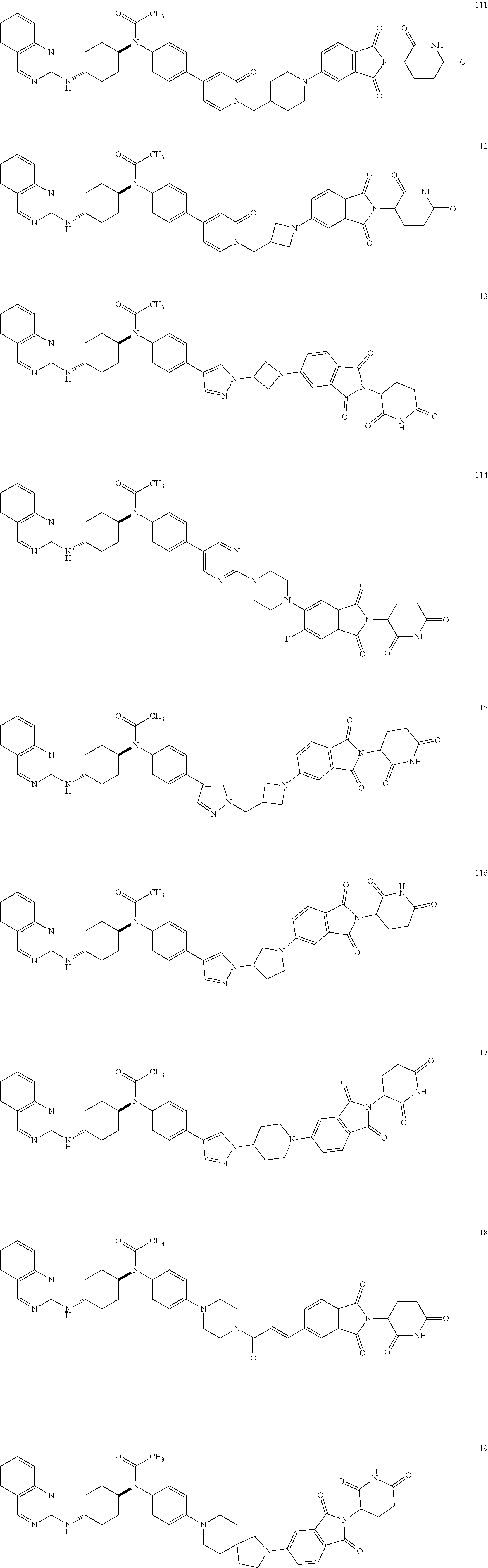

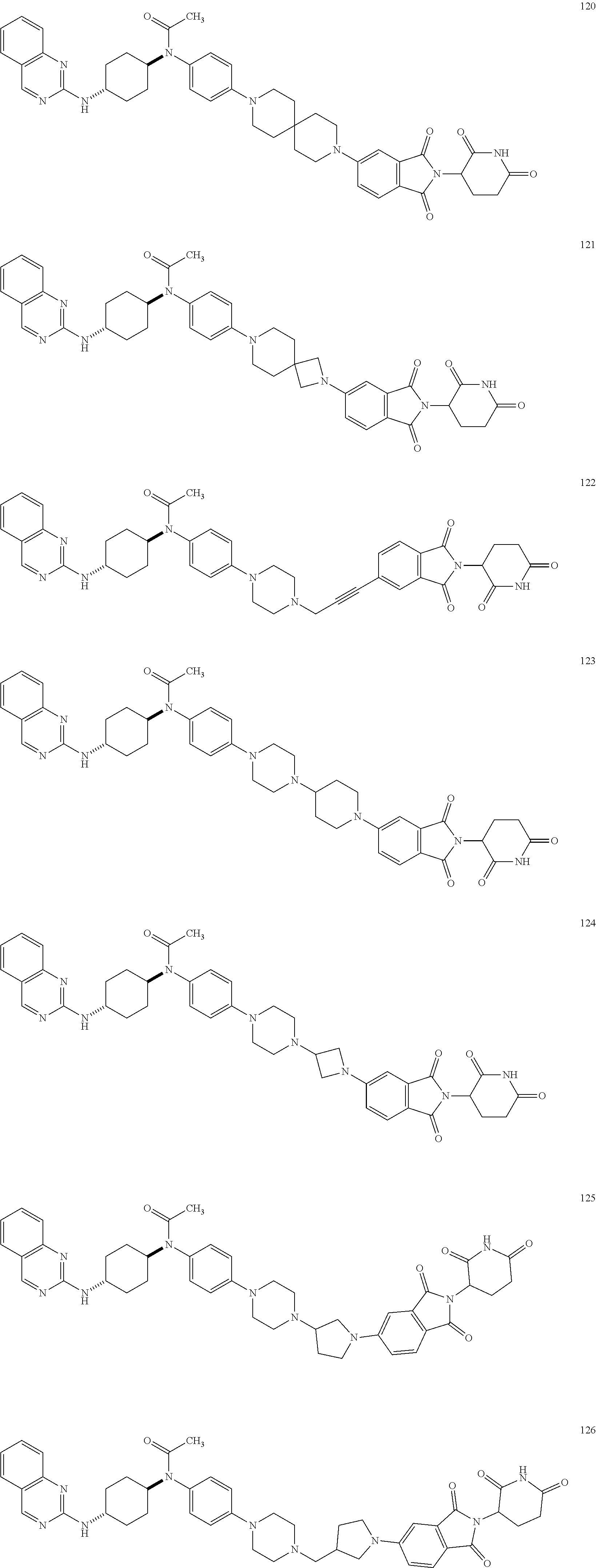

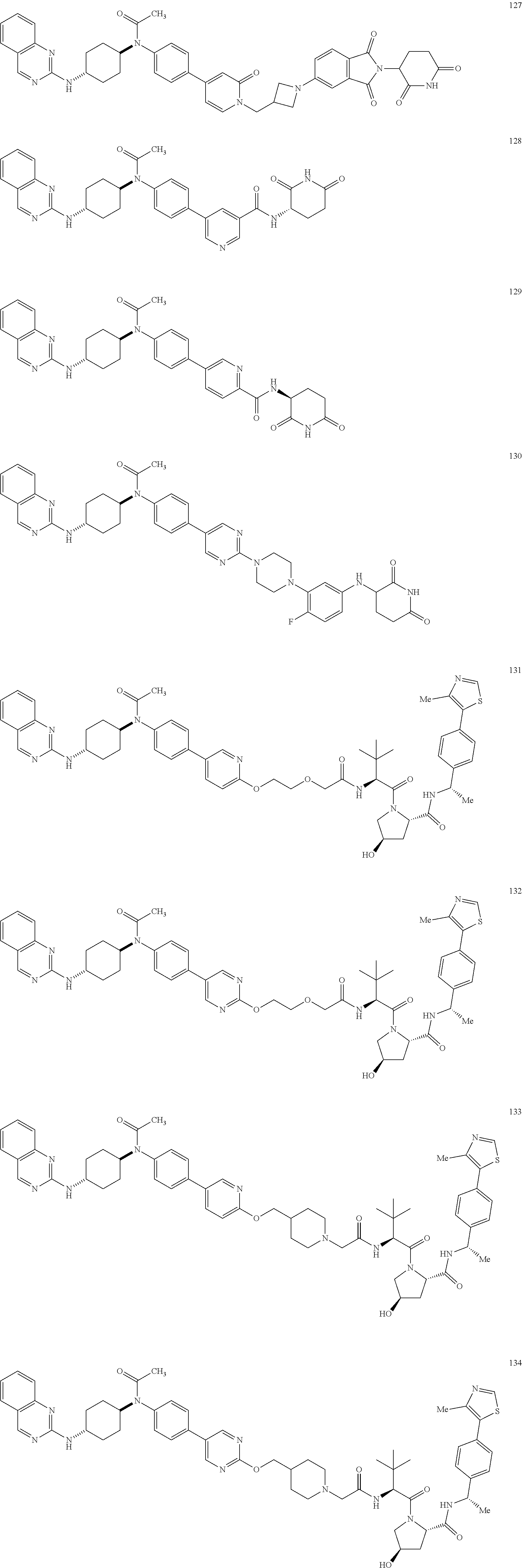

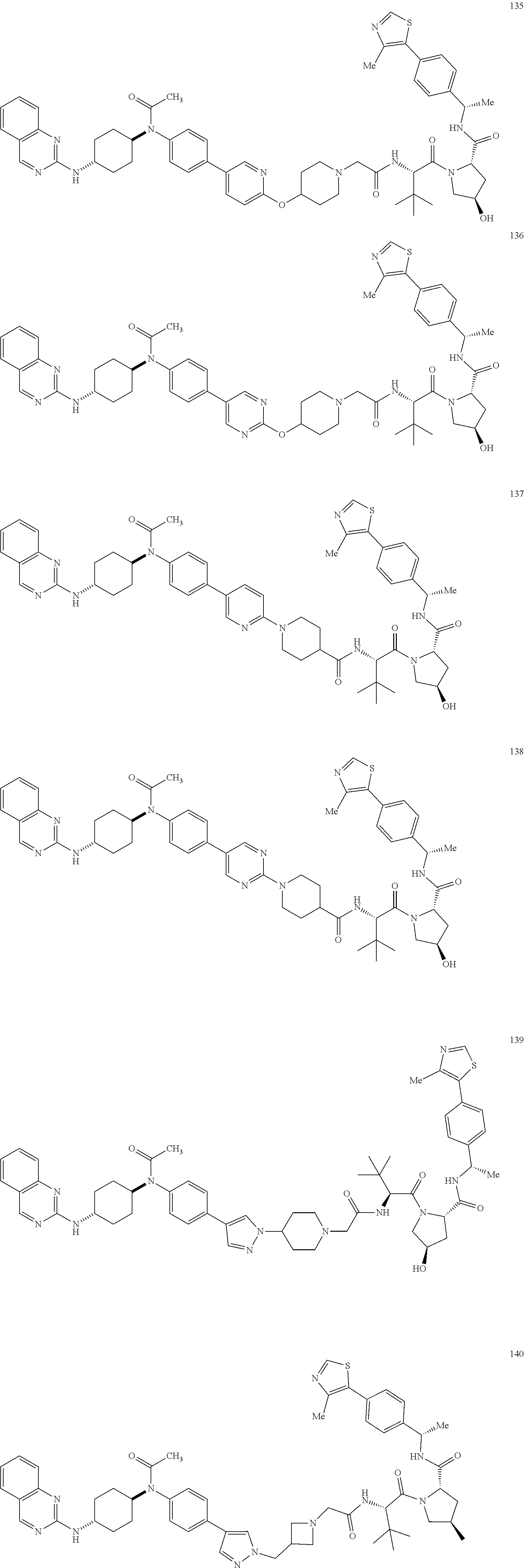

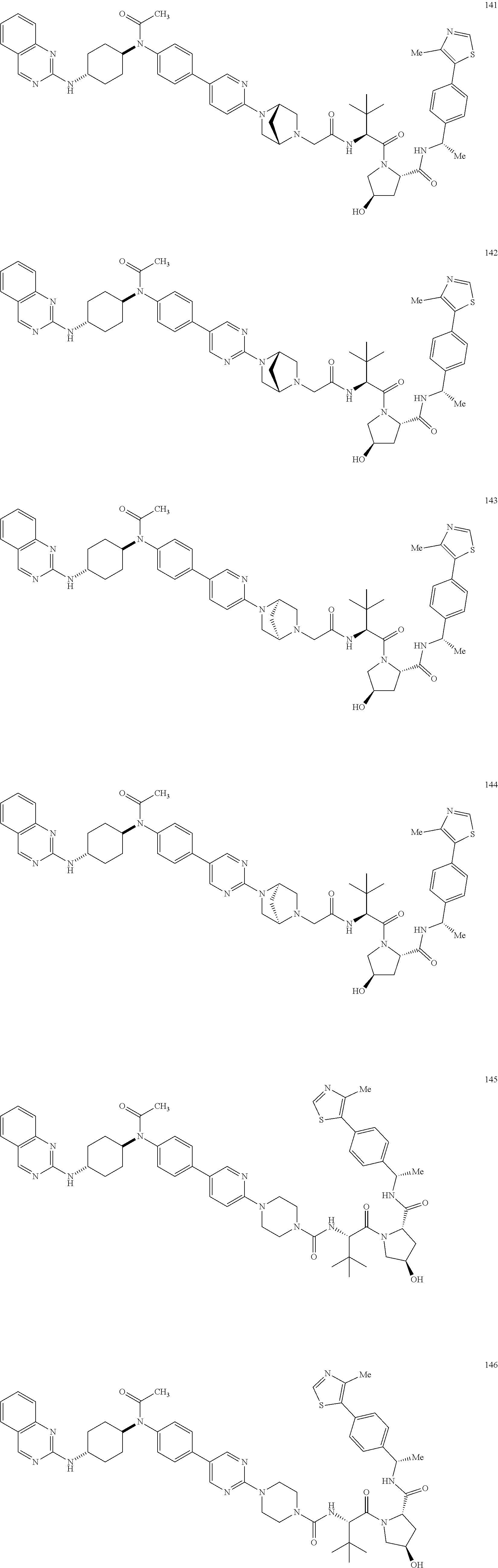

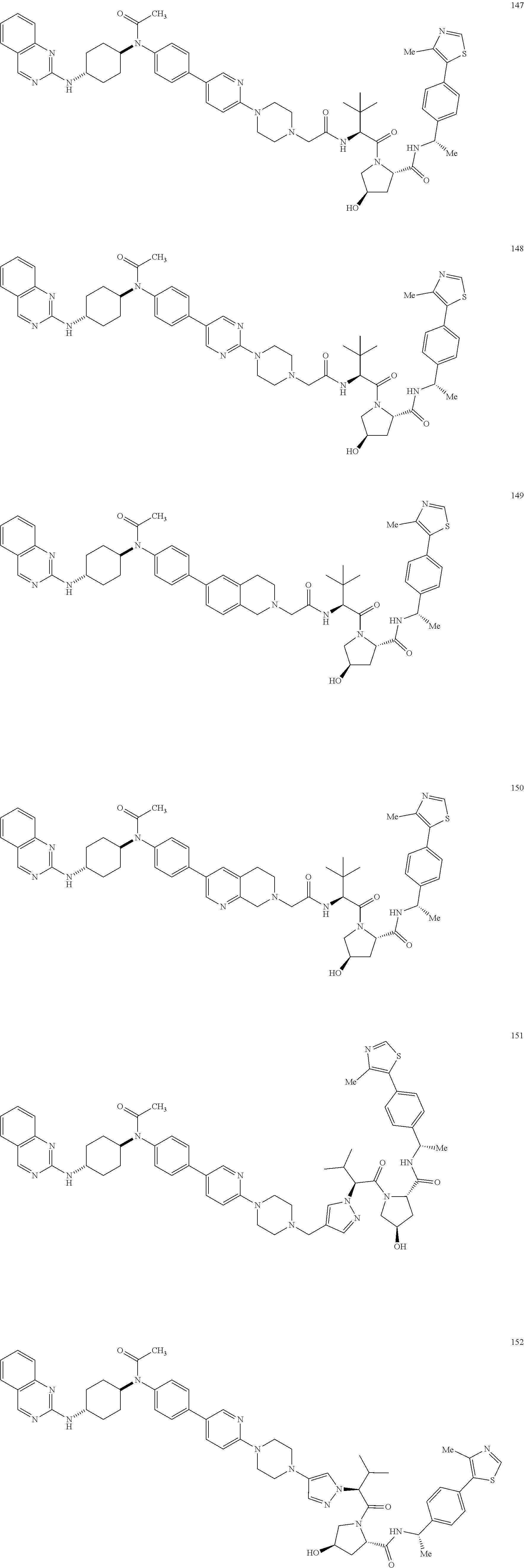

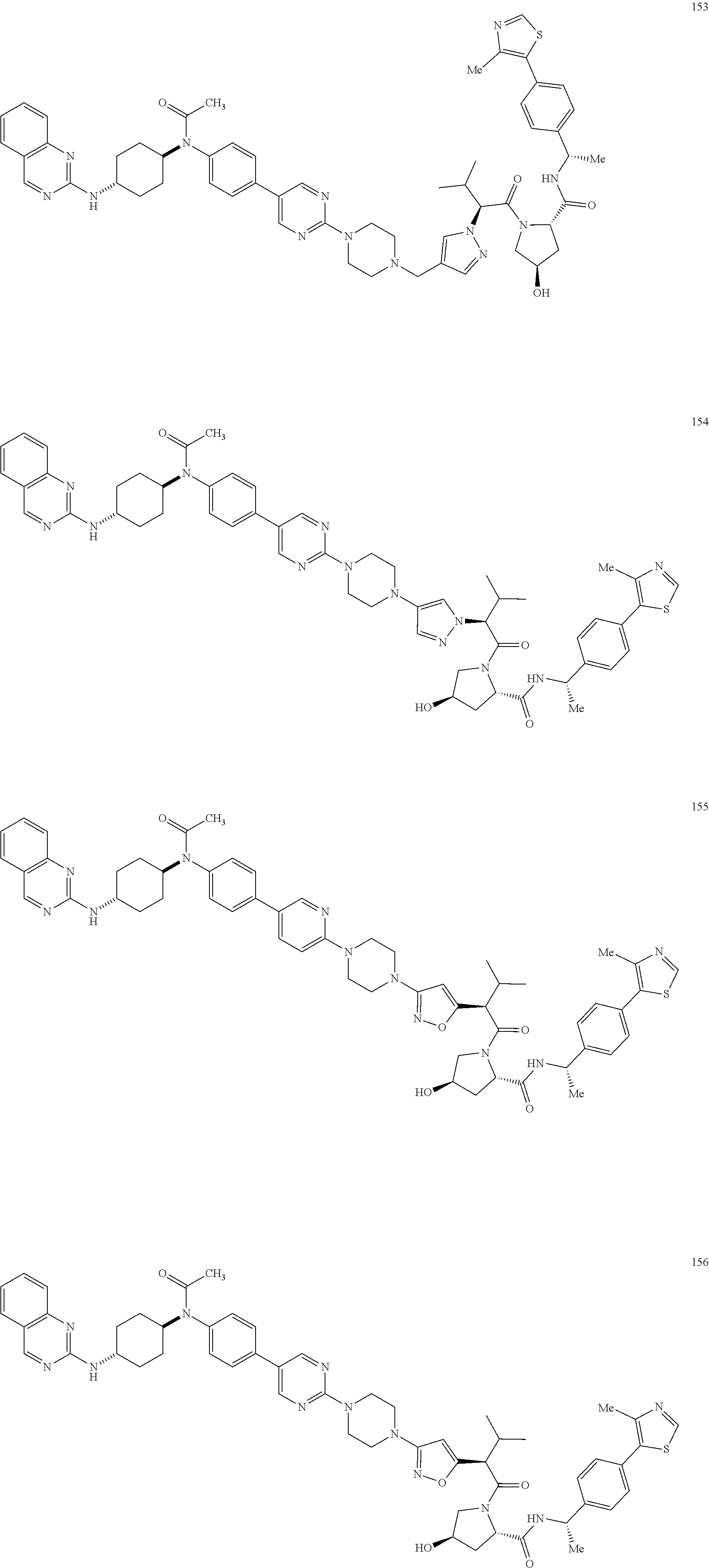

13. The small molecule conjugate according to any one of claim 1, characterized in that, the pharmaceutically acceptable salt is a salt formed with the following acids: citric acid, fumaric acid, acetic acid, oxalic acid, tartaric acid, malic acid, maleic acid, lactic acid, camphorsulfonic acid, p-toluenesulfonic acid, methanesulfonic acid, trifluoromethanesulfonic acid, benzenesulfonic acid, succinic acid, citric acid, hydrohalic acid, sulfuric acid, phosphoric acid, nitric acid, carbonic acid, glutamic acid, and aspartic acid.

14. The small molecule conjugate according to claim 12, characterized in that: the said small molecule conjugate is a compound as shown in any one of the following formulae 71 to 86: 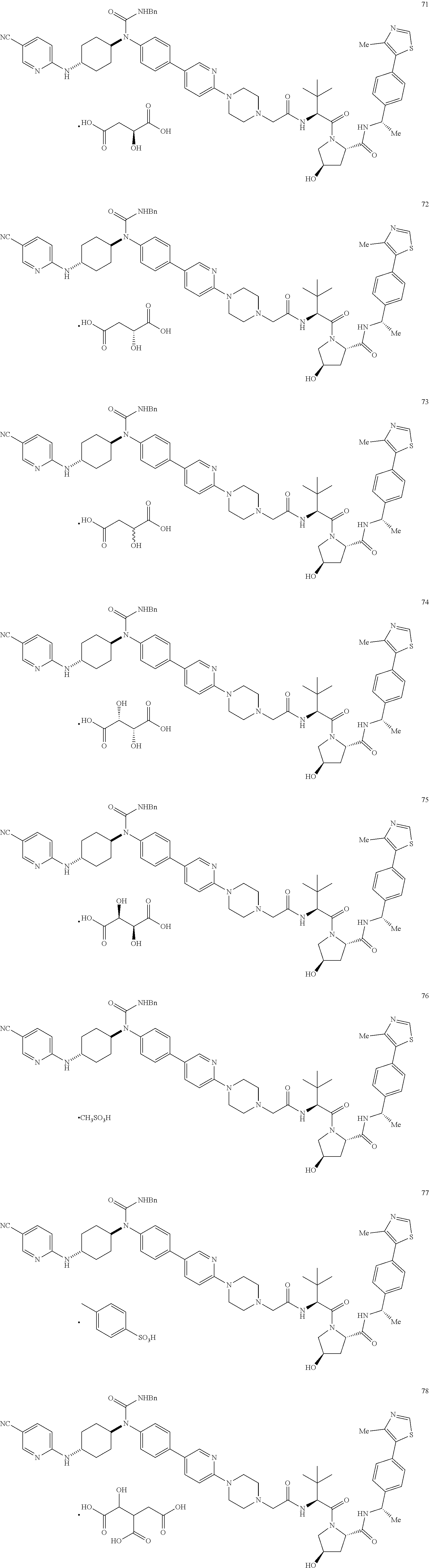 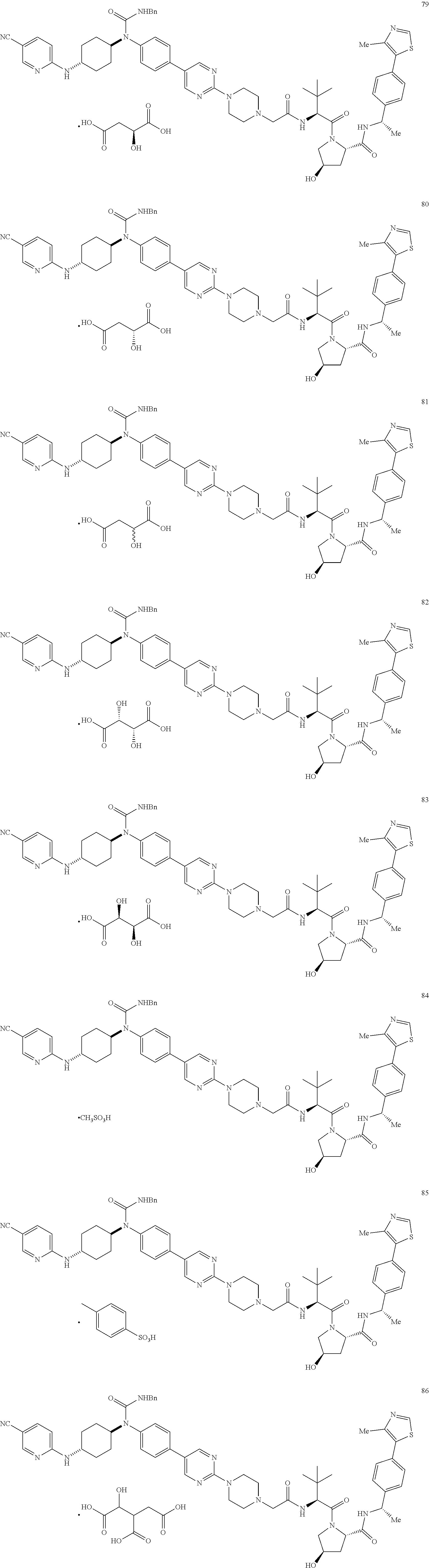
or the following compounds:

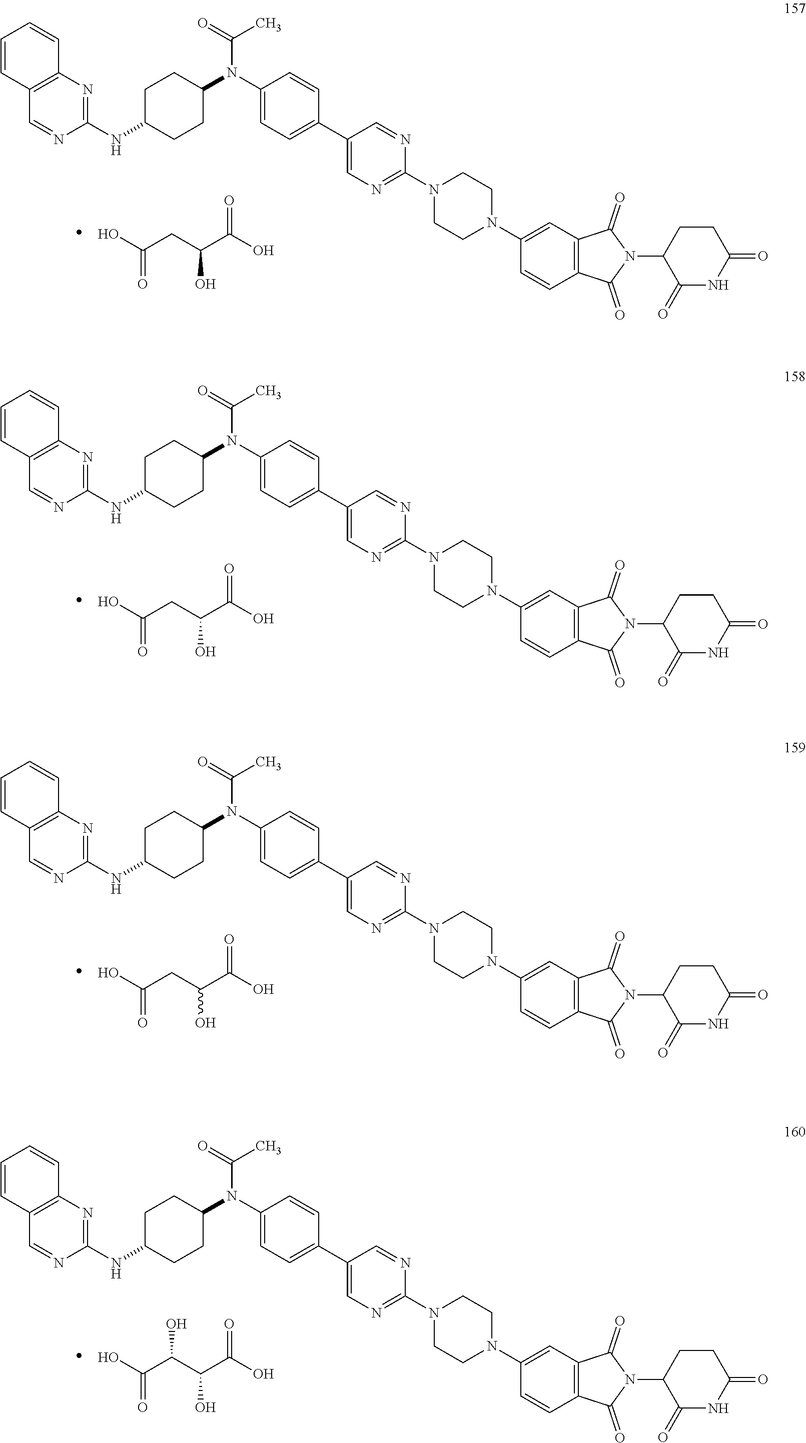

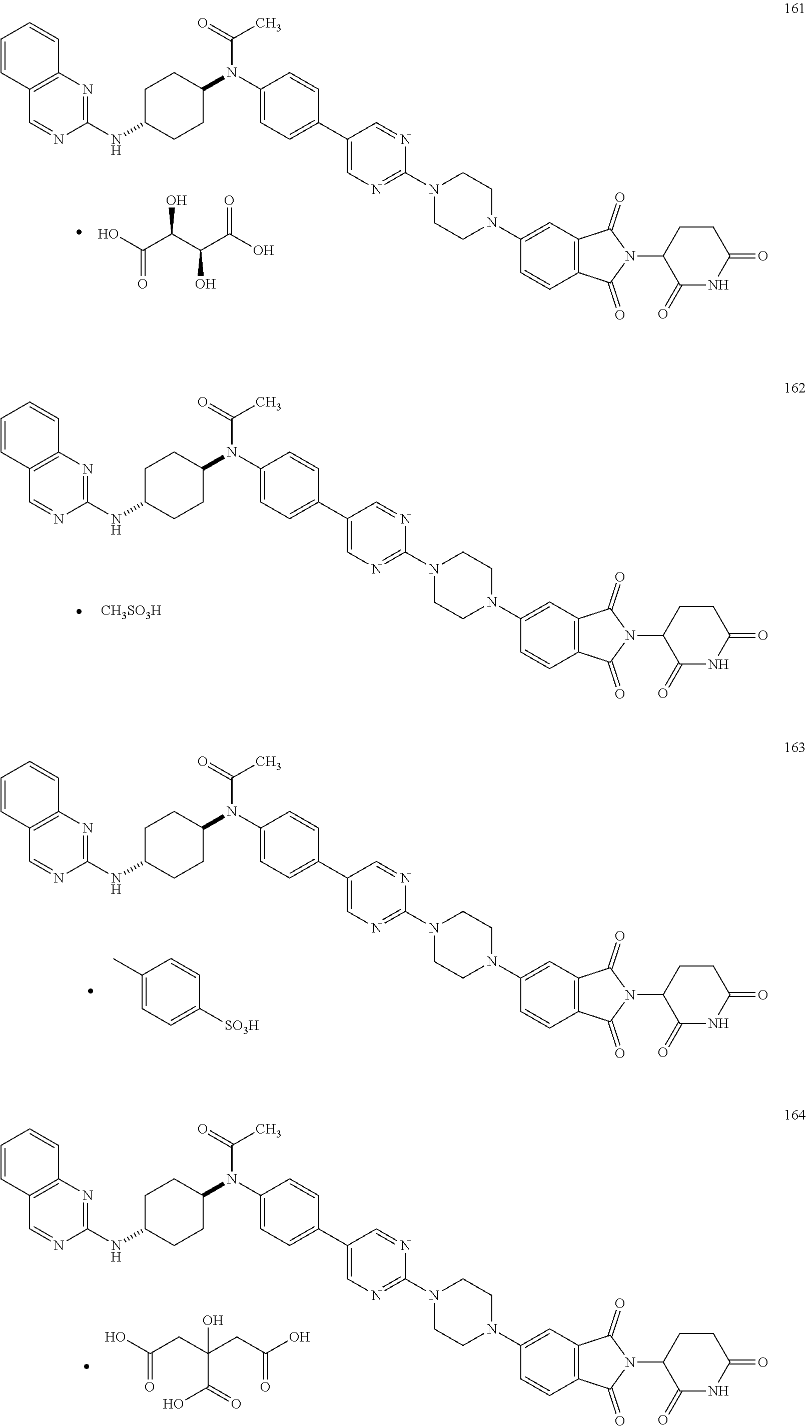

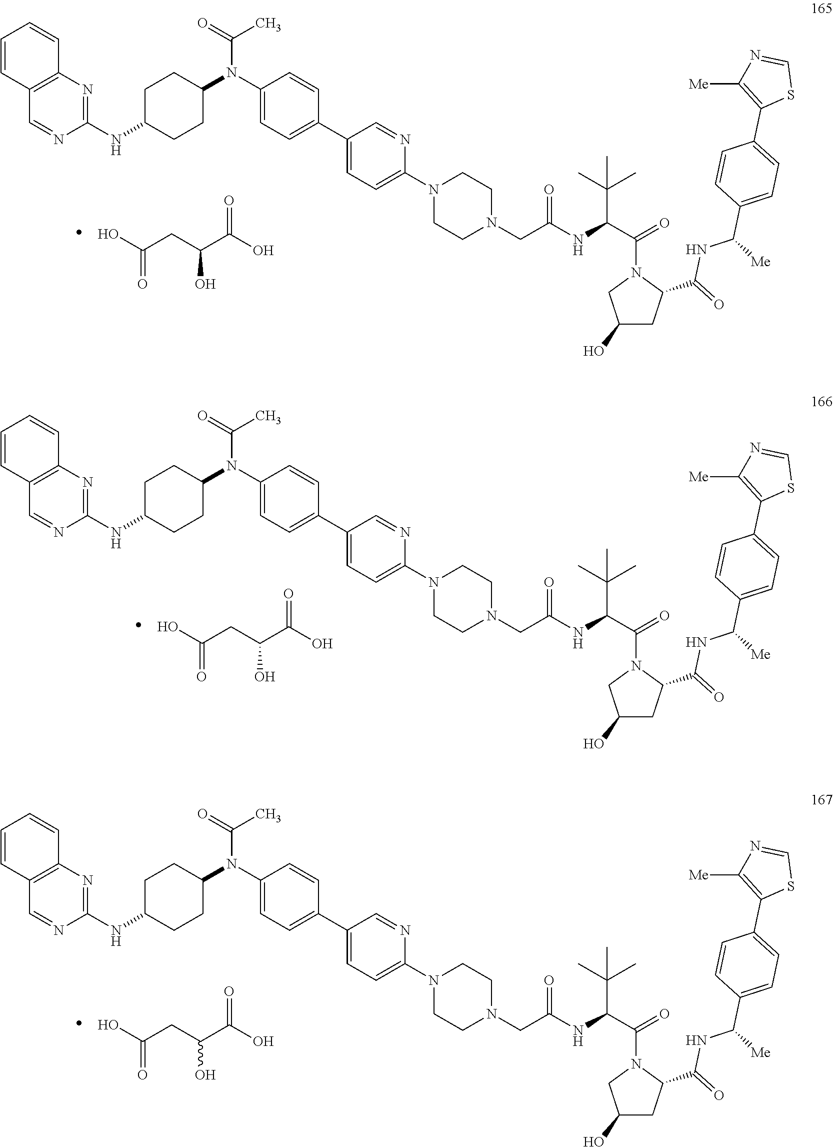

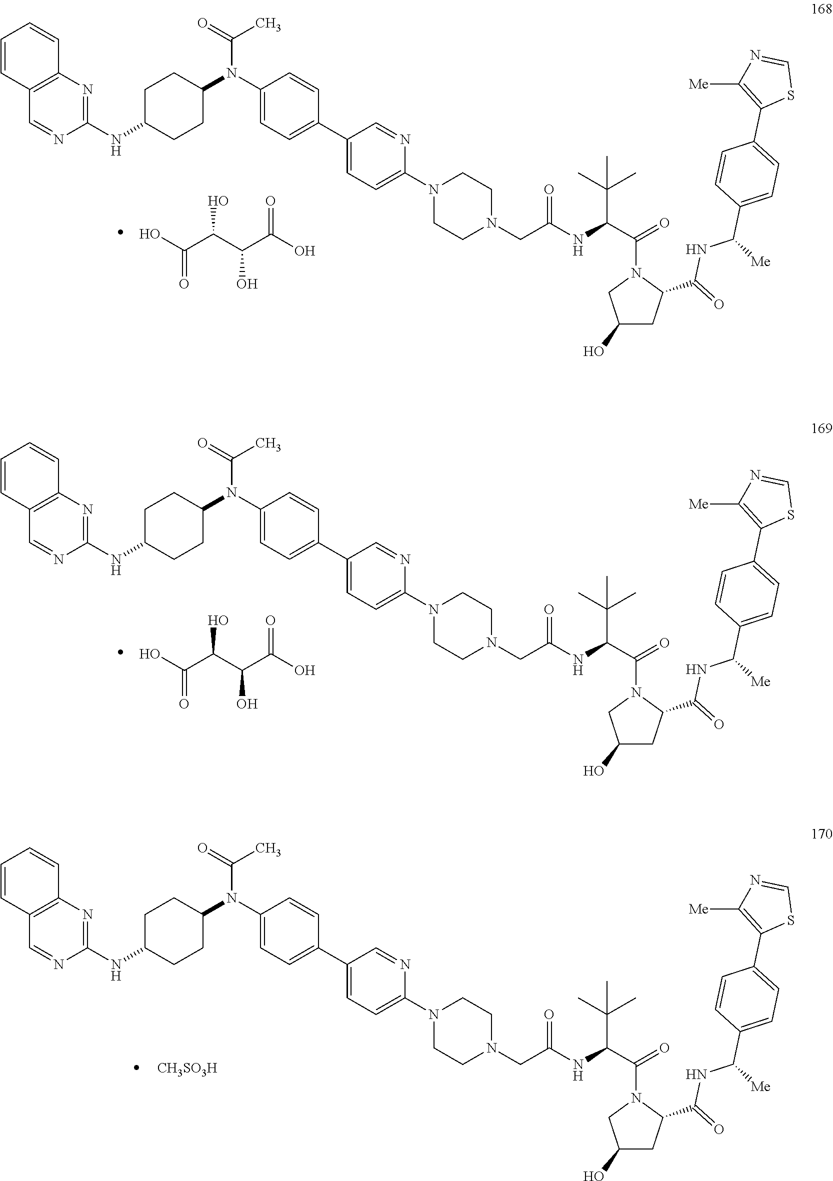

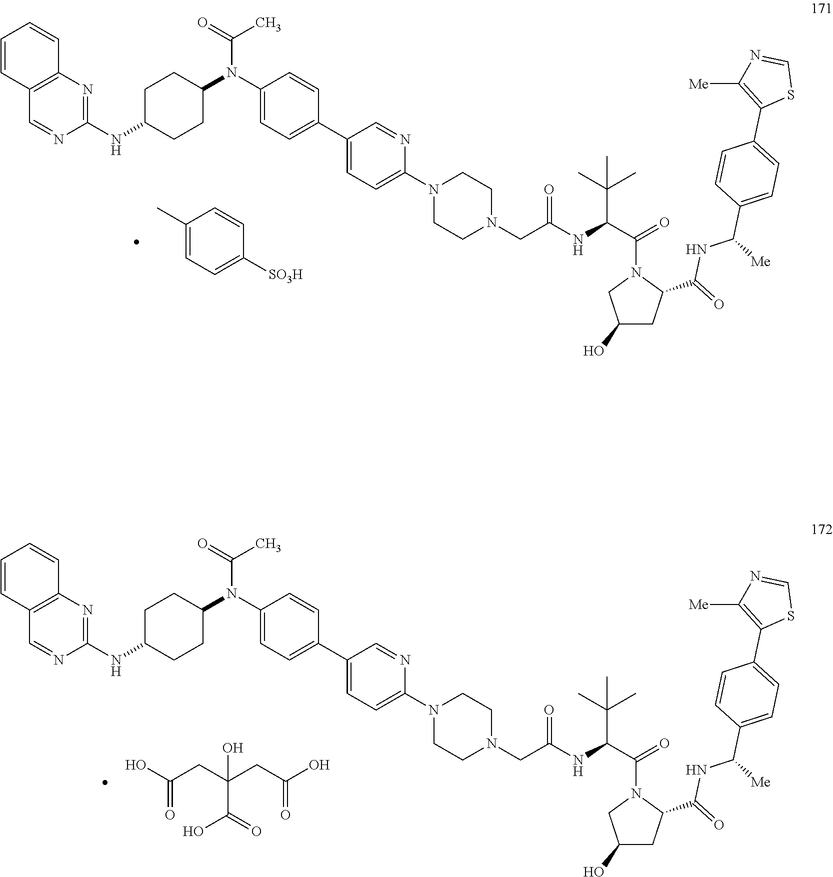 [SMILES: CC(=O)N(c1ccc(-c2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)nc2)cc1)[C@H]1CC[C@H](Nc2ncc3ccccc3n2)CC1 ; Cc1ccc(S(=O)(=O)O)cc1 ; CC(=O)N(c1ccc(-c2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)nc2)cc1)[C@H]1CC[C@H](Nc2ncc3ccccc3n2)CC1 ; O=C(O)CC(O)(CC(=O)O)C(=O)O]

15. A method for preparing the small molecule conjugate according to any one of claim 1, characterized in that, the method comprises the following steps: an E3 ligase ligand precursor compound, a linker precursor compound, and a T-part CDK12/13 protein ligand precursor compound are subjected to an amide condensation reaction, a substitution reaction, a click reaction, or a cross-coupling reaction, respectively, to obtain the small molecule conjugate as shown in Formula I.

16. The method for preparing the small molecule conjugate according to claim 15, characterized in that, the preparation method is as follows:
A method for preparing the small molecule conjugate according to Formula IV: the small molecule conjugate according to Formula IV is obtained through a nucleophilic substitution reaction between a CDK12/13 ligand terminal derivative as shown in Formula a and a Pomalidomide terminal derivative as shown in Formula b; 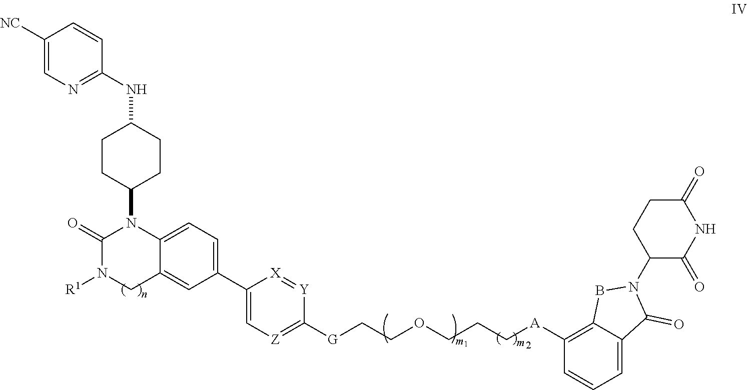
wherein, in the small molecule conjugate according to Formula IV, G is O, A is NH, X is CH, Y is N, and Z is CH; in Formula a, the definitions of R¹, m₁, m₂, and n are the same as in Formula IV; and in Formula b, the definition of B is the same as in Formula IV; 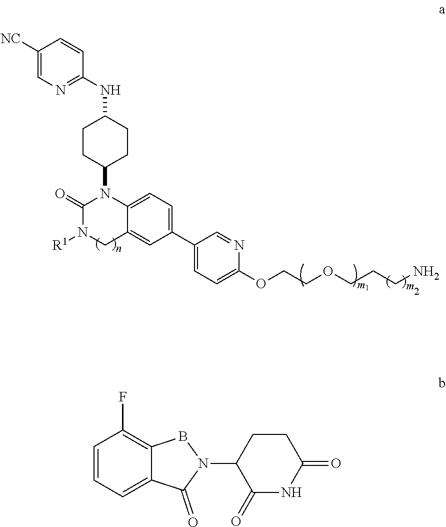
or, a method for preparing the small molecule conjugate according to Formula V, comprising the following steps: the small molecule conjugate according to Formula V is obtained by a nucleophilic substitution reaction between a CDK12/13 ligand terminal derivative as shown in Formula c and a Pomalidomide terminal derivative as shown in Formula b; 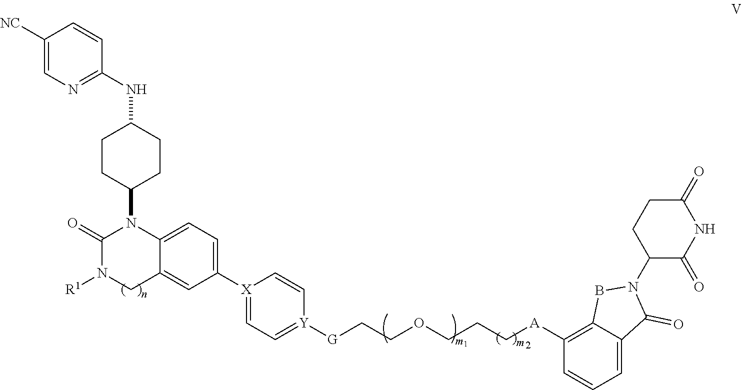
wherein, in the small molecule conjugate according to Formula V, G is CH₂, A is NH, X is N, and Y is N; in Formula c, the definitions of R¹, m₁, m₂, and n are the same as in Formula V; and in Formula b, the definition of B is the same as in Formula V; 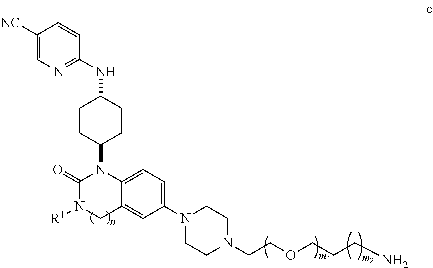
or, a method for preparing the small molecule conjugate according to Formula VI, comprising the following steps: the small molecule conjugate according to Formula VI is obtained by a nucleophilic substitution reaction between a CDK12/13 ligand terminal derivative as shown in Formula d and a Pomalidomide terminal derivative as shown in Formula b; 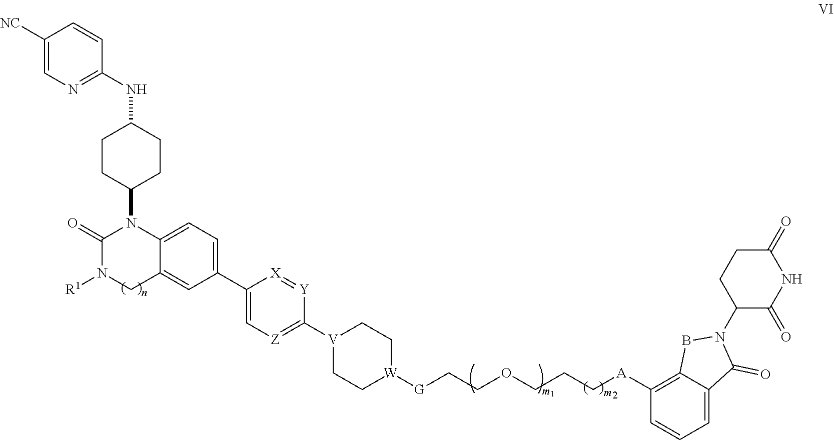
wherein, in the small molecule conjugate according to Formula VI, G is CH₂, A is NH, X is CH, Y is N, Z is CH, V is N, and W is N; in Formula d, the definitions of R¹, m₁, m₂, and n are the same as in Formula VI; and in Formula b, the definition of B is the same as in Formula VI. 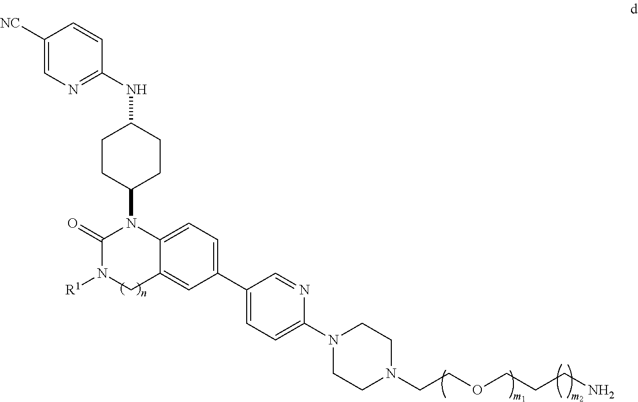
or, a method for preparing the small molecule conjugate according to Formula VII, comprising the following steps: the small molecule conjugate according to Formula VII is obtained by a condensation reaction between a CDK12/13 ligand terminal derivative as shown in Formula e and a VHL ligand derivative as shown in Formula f, 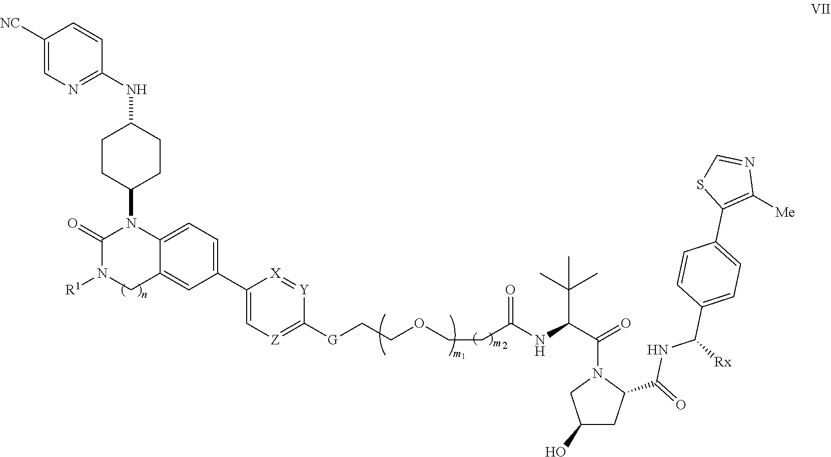
wherein, in the small molecule conjugate according to Formula VII, G is O, X is CH, Y is N, and Z is CH; in Formula e, the definitions of R¹, m₁, m₂, and n are the same as in Formula VII; and in Formula f, the definition of Rₓ is the same as in Formula VII; 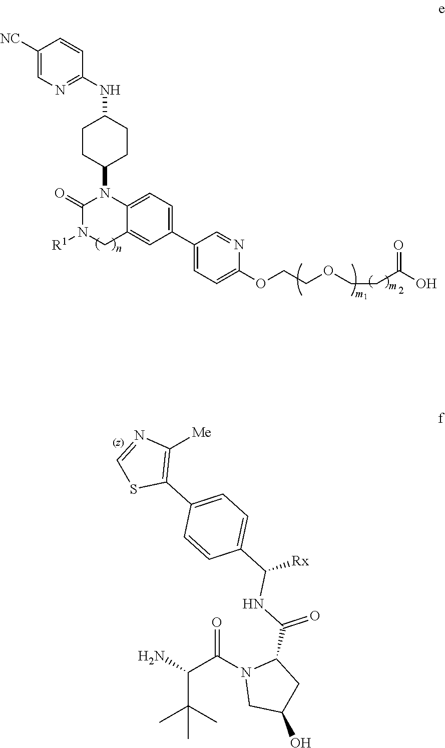
or, a method for preparing the small molecule conjugate according to Formula VIII, comprising the following steps: the small molecule conjugate according to Formula VIII is obtained by a condensation reaction between a CDK12/13 ligand terminal derivative as shown in Formula g and a VHL ligand derivative as shown in Formula f, 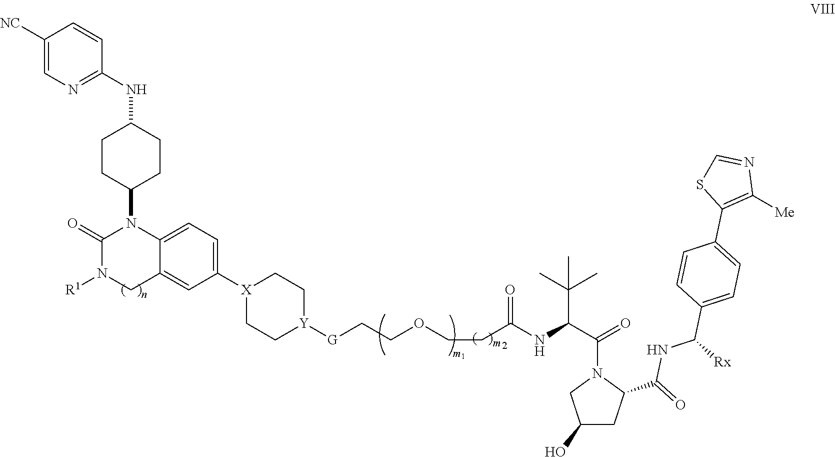
wherein, in the small molecule conjugate according to Formula VIII, G is CH₂, X is N, and Y is N; in Formula g, the definitions of R¹, m₁l, m₂, and n are the same as in Formula VIII; and in Formula f, the definition of Rₓ is the same as in Formula VIII. 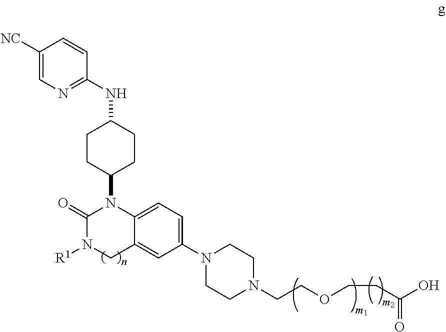
or, a method for preparing the small molecule conjugate according to Formula IX, comprising the following steps: the small molecule conjugate according to Formula IX is obtained by a condensation reaction between a CDK12/13 ligand terminal derivative as shown in Formula h and a VHL ligand derivative as shown in Formula f, 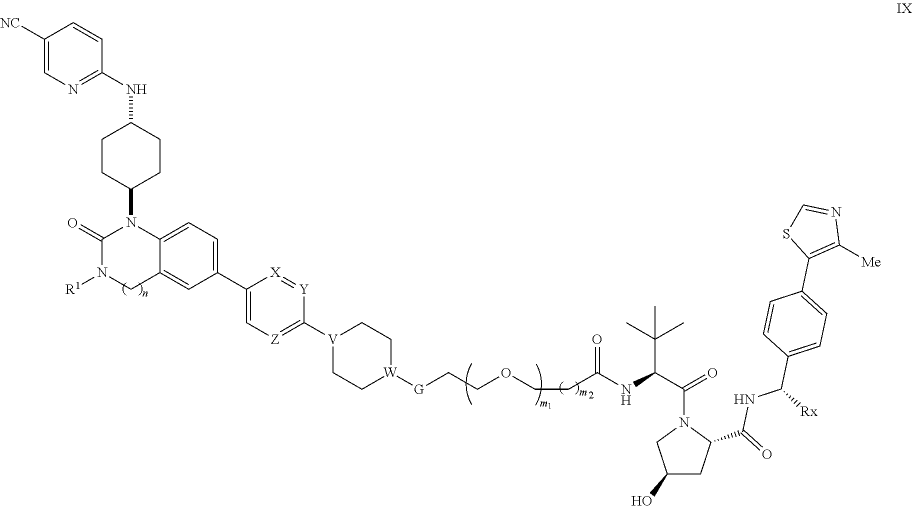
wherein, in the small molecule conjugate according to Formula IX, G is CH₂, X is CH, Y is N, Z is CH, V is N, and W is N; in Formula h, the definitions of R¹, m₁, m₂, and n are the same as in Formula IX; and in Formula f, the definition of Rₓ is the same as in Formula IX. 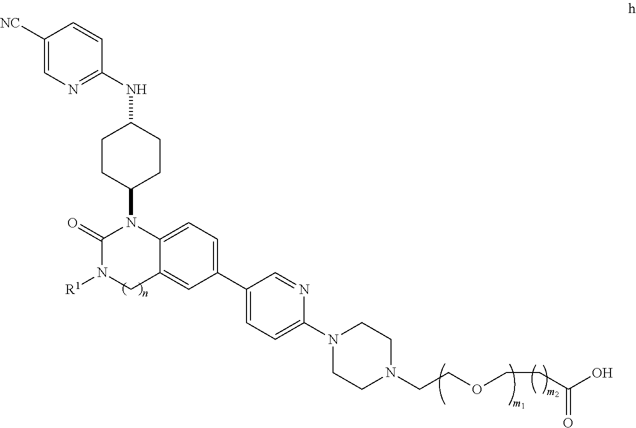
or, a method for preparing the small molecule conjugate according to Formula X, comprising the following steps: the small molecule conjugate according to Formula X is obtained by a condensation reaction between a CDK12/13 ligand terminal derivative as shown in Formula e and an MDM2 ligand derivative as shown in Formula j; 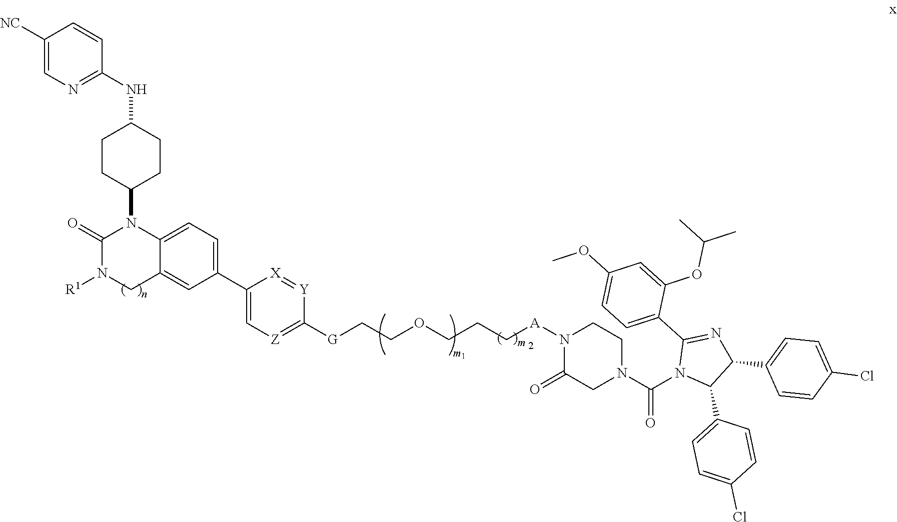
wherein, in the small molecule conjugate according to Formula X, G is O, X is CH, Y is N, Z is CH, and A is CO; and in Formula e, the definitions of R¹, m₁, m₂, and n are the same as in Formula X. 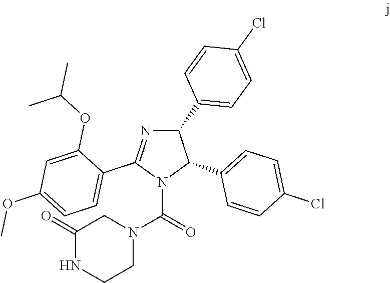 [SMILES: COc1ccc(C2=N[C@H](c3ccc(Cl)cc3)[C@H](c3ccc(Cl)cc3)N2C(=O)N2CCNC(=O)C2)c(OC(C)C)c1]
or, a method for preparing the small molecule conjugate according to Formula XI, comprising the following steps: the small molecule conjugate according to Formula XI is obtained by a condensation reaction between a CDK12/13 ligand terminal derivative as shown in Formula g and an MDM2 ligand derivative as shown in Formula j;
wherein, in the small molecule conjugate according to Formula XI, G is CH₂, X is N, Y is N, and A is CO; and in Formula g, the definitions of R¹, m₁, m₂, and n are the same as in Formula XI;
or, a method for preparing the small molecule conjugate according to Formula XII, comprising the following steps: the small molecule conjugate according to Formula XII is obtained by a condensation reaction between a CDK12 ligand terminal derivative as shown in Formula h and an MDM2 ligand derivative as shown in Formula j;
wherein, in the small molecule conjugate according to Formula XII, G is CH₂, X is CH, Y is N, Z is CH, V is N, W is N, and A is CO; and in Formula h, the definitions of R¹, m₁, m₂, and n are the same as in Formula XII.

17. (canceled)

18. (canceled)

19. (canceled)

20. A composition, characterized in that, it comprises a small molecule conjugate according to any one of claim 1 and one or more pharmaceutically acceptable carriers.

21. The composition according to claim 20, characterized in that, the said carrier comprises one or more of the following conventional in the pharmaceutical field: diluents, excipients, fillers, binders, wetting agents, disintegrants, absorption enhancers, surfactants, adsorbent carriers, and lubricants.

22. The composition according to claim 20 including tablets, powders, granules, capsules, oral liquids, pastes, creams, and injections.

23. The use of a compound according to any one of claim 1 in the preparation of a medicament for single or combined use for the prevention and/or treatment of a disease that benefits from the inhibition or degradation of CDK12 and/or CDK13.

24. The use according to claim 23, characterized in that, the said disease is a disease that benefits from the degradation or inhibition of CDK12/13 in one or more of the following cells:
colorectal cancer cells, cervical cancer cells, ovarian cancer cells, breast cancer cells, glioma cells, acute myeloid leukemia cells, T-lymphocyte leukemia cells, myeloma cells, gastric cancer cells, and liver cancer cells.

25. The use according to claim 23, characterized in that, the said disease includes one or more of the following: breast cancer, prostate cancer, gastric cancer, cervical cancer, ovarian cancer, Ewing's sarcoma, leukemia, and leishmaniasis.
